(19) United States
(12) Patent Application Publication
(10) Pub. No.: US 2024/0383852 A1
(43) Pub. Date: Nov. 21, 2024

(54) ANTI-VIRAL COMPOUNDS

(71) Applicant: INFEX Therapeutics Limited, Macclesfield Cheshire (GB)

(72) Inventors: James Kirkham, Cheshire (GB); Ian Cooper, Cheshire (GB); Adam Bunt, Cheshire (GB); Kevin Blades, Cheshire (GB); David Orr, Cheshire (GB); Andrew Wilkinson, Cheshire (GB); Paul Schofield, Cheshire (GB)

(21) Appl. No.: 18/281,038

(22) PCT Filed: Mar. 11, 2022
(86) PCT No.: PCT/GB22/50644

§ 371 Date: Sep. 8, 2023

(30) Foreign Application Priority Data
Mar. 12, 2021 (GB) 2103461.6
Nov. 9, 2021 (GB) 2116106.2

(51) Int. Cl. C07D 211/14; A61K 31/36; A61K 31/397; A61K 31/407; A61K 31/415; A61K 31/4375; A61K 31/4418; A61K 31/4439; A61K 31/451; A61K 31/495; A61K 31/4995; A61K 31/505; A61K 31/506; A61K 31/551; C07D 211/12; C07D 213/02; C07D 213/38; C07D 213/56; C07D 231/12; C07D 239/42; C07D 243/08; C07D 285/08; C07D 295/033; C07D 317/50; C07D 333/20; C07D 403/04; C07D 471/18; C07D 487/04; C07D 487/10

(52) U.S. Cl. CPC C07D 211/14; A61K 31/36; A61K 31/397; A61K 31/407; A61K 31/415; A61K 31/4375; A61K 31/4418; A61K 31/4439; A61K 31/451; A61K 31/495; A61K 31/4995; A61K 31/505; A61K 31/506; A61K 31/551; C07D 211/12; C07D 213/02; C07D 213/38; C07D 213/56; C07D 231/12; C07D 239/42; C07D 243/08; C07D 285/08; C07D 295/033; C07D 317/50; C07D 333/20; C07D 403/04; C07D 471/18; C07D 487/04; C07D 487/10

(57) ABSTRACT

This invention relates to compounds that can be used to treat viral infections. The compounds are papain-like protease (PLpro) inhibitors.

This invention relates to compounds that can be used to treat viral infections. The novel compounds of the present invention are enzyme inhibitors and more particularly are papain-like protease (PLpro) inhibitors.

Viral infections have the ability to spread through populations so rapidly that they give rise to epidemics or pandemics. Such occurrences and becoming increasingly common. The most recent example of this was the coronavirus disease 2019 (COVID-19) pandemic caused by the SARS-CoV-2 virus, that caused death or severe illness in millions of people worldwide and significantly impacted global economies.

Papain-like protease (PLpro) is one of two cysteine proteases that reside within viral polyprotein and is responsible for processing the polyprotein into its functional units. These functional units in turn assemble into complexes to execute viral RNA synthesis. PLpro is therefore essential for viral replication (Nature, 2020, 587, 657-662).

PLpro is conserved across many coronaviruses, including SARS-CoV-1, MERS-CoV and SARS-CoV-2, with high homology seen between species/strains (ACS Infect. Dis., 2020, 6, 8, 2099-2109). If PLPro can be selectively inhibited, it could prevent viral replication and be used in the treatment of viral infections arising from these species and strains.

WO2010/022355A1 discloses compounds and compositions for treating respiratory disease and illness, such as SARS. The compounds disclosed therein show inhibition of SARS-Cov-1 PLpro.

Recent research has shown that the PLpro binding sites for SARS-CoV-1 and SARS-CoV-2 are highly homogenous (ACS Infect. Dis., 2020, 6, 8, 2099-2109).

Shen et al. (https://www.biorxiv.org/content/10.1101/2021.02.13.431008v1) discloses potent non-covalent inhibitors of SARS-CoV-2 PLpro which are shown to block viral replication in monkey and human cell cultures.

It is an aim of the present invention to provide new compounds which show anti-viral activity, and in particular which inhibit the activity of PLpro.

### BRIEF SUMMARY OF THE DISCLOSURE

In a first aspect of the present invention is provided a compound of formula (I) or pharmaceutically acceptable salt thereof:

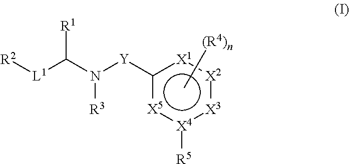

wherein
Y is —C(O)—, —C(S)—, —C(═NR⁶)—;
-L¹- is absent or a linker selected from C₁ alkylene, C₂-alkenylene, or C₂-alkynylene;
X¹ is absent or is selected from carbon and nitrogen;
X², X³ and X⁵ are each independently selected from carbon, nitrogen, oxygen and sulfur;
X⁴ is selected from carbon and nitrogen;
wherein when X¹ is carbon or nitrogen, X⁴ is carbon, X², X³ and X⁵ are each independently selected from carbon and nitrogen, and no more than two of X¹, X², X³ and X⁵ may be nitrogen,
wherein when X¹ is absent, no more than two of X², X³, X⁴ and X⁵ may be nitrogen and no more than one of X², X³ and X⁵ may be oxygen or sulfur;
R¹ is selected from the group comprising: C₁ or C₂ alkyl, C₁ or C₂ haloalkyl, and C₁ or C₂ alkylene-R^{1a}; wherein R^{1a} is selected from OR⁶, SR⁶, NR⁶R⁷, CO₂R⁶ and CONR⁶R⁶;
R² is selected from phenyl, 5- or 6-membered heteroaryl, 5- or 6-membered heterocycloalkyl or C₅ or C₆ cycloalkyl, and said phenyl, heteroaryl or cycloalkyl is optionally fused to or substituted with a group selected from phenyl, 5- or 6-membered heteroaryl, 5- or 6-membered heterocycloalkyl or C₅ or C₆ cycloalkyl; wherein any said phenyl or heteroaryl group is optionally substituted with at least one R⁸ group; or wherein any said heterocycloalkyl or cycloalkyl is optionally substituted with at least one R⁹ group;
R³, R⁶ and R¹¹ are each independently at each occurrence selected from the group comprising: H and C₁-C₆-alkyl;
R⁴ is independently at each occurrence selected from the group comprising: halo, C₁-C₆-alkyl, C₁-C₆-haloalkyl, C₁-C₆-alkylene-R¹⁰, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₃₋₆ cycloalkyl, C₂₋₆-alkenyl, C₂₋₆-alkynyl, phenyl and 5- or 6-membered heteroaryl;
R⁵ is selected from the group comprising: —C(O)NR⁶R¹⁴, —C(O)R¹², phenyl, 5- or 6-membered heteroaryl; 5-, 6-, 7-, 8-, 9- or 10-membered heterocycloalkyl and cyclopropyl; wherein any said phenyl or heteroaryl group is optionally substituted with at least one R⁸ group; or wherein any said heterocycloalkyl or cyclopropyl is optionally substituted with at least one R⁹ group;
wherein when X⁴ is nitrogen, R⁵ is selected such that R⁵ is attached to X⁴ via a carbon atom;
R⁷ is independently at each occurrence selected from the group comprising: H, C₁-C₆-alkyl, C(O)—C₁-C₆-alkyl and S(O)₂—C₁-C₆-alkyl;
R⁸ is independently at each occurrence selected from the group comprising: halo, C₁-C₆-alkyl, C₁-C₆-haloalkyl, C₁-C₆-alkylene-R¹⁰, —OR¹⁰, cyano, nitro, —NR⁶R⁷, —SR¹⁰, C(O)R¹⁰, C(O)OR¹⁰, C(O)NR⁶R¹⁰, —S(O)R¹⁰, —S(O)₂R¹⁰, —S(O)₂NR⁶R¹⁰, C₃₋₆ cycloalkyl, C₂₋₆-alkenyl, C₂₋₆-alkynyl, 5-, 6-, 7-, 8-, 9- or 10-membered heterocycloalkyl, phenyl and 5- or 6-membered heteroaryl; wherein where R⁸ is heterocycloalkyl, phenyl or heteroaryl, R⁸ is optionally substituted where chemically possible with one or more R^{8c} groups;
R^{8c} is independently selected at each occurrence from: halo, C₁-C₆-alkyl, C₁-C₆-haloalkyl, C₁-C₆-alkylene-R¹⁰, C₁-C₆-alkylene-NR⁶R¹⁰, —OR¹⁰, C(O)R¹⁰, C(O)OR¹⁰, C(O)NR⁶R¹⁰;
R⁹ is independently at each occurrence selected from the group comprising: ═O, ═S, halo, C₁-C₆-alkyl, C₁-C₆-haloalkyl, —OR¹⁰, cyano, nitro, —NR⁶R⁷, —NR¹¹R¹², —SR¹⁰, C(O)R¹⁰, C(O)OR¹⁰, C(O)NR⁶R¹⁰, —S(O)R¹⁰, —S(O)₂R¹⁰, —S(O)₂NR⁶R¹⁰, C₃₋₆ cycloalkyl, 4-, 5- or 6-membered heterocycloalkyl, C₂₋₆-alkenyl C₂₋₆-alkynyl and C₁-C₃-alkylene-R^{9a}; wherein R^{9a} is selected from OR⁶, SR⁶, S(O)₂R⁶, S(O)₂NR⁶R⁶, S(O)₂Ph, NR⁶R⁷, CO₂R⁶, CONR⁶R⁶, 4-, 5- or 6-membered heterocycloalkyl, and cyclopropyl;
R¹⁰ is independently selected at each occurrence from the group comprising: H, C₁-C₆-alkyl, C₁-C₆-haloalkyl, C₁-C₆-alkylene-R¹⁰, C₃₋₈ cycloalkyl, 4-, 5-, 6-, 7- or 8-membered heterocycloalkyl, phenyl and 5- or 6-membered heteroaryl;
R^{10a} is independently selected at each occurrence from C₃₋₈ cycloalkyl, OR⁶, SR⁶, S(O)₂R⁶, S(O)₂Ph, NR⁶R⁷, CO₂R⁶, CONR⁶R⁶, phenyl, 5- or 6-membered heteroaryl, and 5- or 6-membered heterocycloalkyl;
R¹² is 6-membered heterocycloalkyl; wherein said heterocycloalkyl is optionally substituted with at least one R¹³ group;
R¹³ is independently at each occurrence selected from: ═O, ═S, halo, C₁-C₆-alkyl, C₁-C₆-haloalkyl, —OR⁶, cyano, nitro, —NR⁶R⁷, —SR⁶, C(O)R⁶, C(O)OR⁶, C(O)NR⁶R⁶, —S(O)R⁶, —S(O)₂R⁶, —S(O)₂NR⁶R⁶, C₃₋₆ cycloalkyl, C₂₋₆-alkenyl, C₂₋₆-alkynyl and C₁-C₃-alkylene-R^{13a}; wherein R^{13a} is selected from OR⁶, SR⁶, S(O)₂R⁶, S(O)₂Ph, NR⁶R⁷, CO₂R⁶ and CONR⁶R⁶;
R¹⁴ is selected from H and C₁-C₃-alkylene-R^{14a}; wherein R^{14a} is selected from OR⁶, SR⁶, S(O)₂R⁶, S(O)₂Ph, NR⁶R⁷, CO₂R⁶ and CONR⁶R⁶; and
n is an integer selected from 0, 1, 2, 3 or 4;
wherein any aforementioned alkyl, alkenyl, alkynyl, haloalkyl, cycloalkyl, heterocycloalkyl, phenyl, heteroaryl, alkylene, alkenylene, alkynylene, C(O)-alkyl and S(O)₂-alkyl is optionally substituted, where chemically possible, by 1 to 4 substituents which are each independently selected at each occurrence from the group consisting of: ═O; ═NR^{a}, ═NOR^{a}, C₁-C₄-alkyl, halo, nitro, cyano, C₁-C₄-haloalkyl, C₂-C₄-alkenyl, C₂-C₄-alkynyl, NR^{a}R^{b}, S(O)₂R^{a}, S(O)R^{a}, S(O)(NR^{a})R^{a}, S(O)₂NR^{a}R^{a}, CO₂R^{a}, C(O)R^{a}, CONR^{a}R^{a}, OR^{a} and SR^{a};
wherein R^{a} is independently selected from H and C₁-C₄-alkyl; and R^{b} is independently selected from H, C₁-C₄-alkyl, C(O)—C₁-C₄-alkyl and S(O)₂—C₁-C₄-alkyl.

In an embodiment of the first aspect, there is provided a compound of formula (Ia) or pharmaceutically acceptable salt thereof:

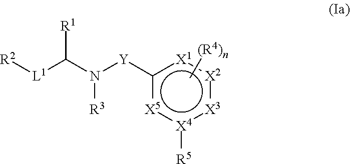

wherein
Y is —C(O)—, —C(S)—, —C(═NR⁶)—;
-L¹- is absent or a linker selected from C₁ alkylene, C₂-alkenylene, or C₂-alkynylene;
X¹ is absent or is selected from carbon and nitrogen;
X², X³ and X⁵ are each independently selected from carbon, nitrogen, oxygen and sulfur;
X⁴ is selected from carbon and nitrogen;
wherein when X¹ is carbon or nitrogen, X⁴ is carbon, X², X³ and X⁵ are each independently selected from carbon and nitrogen, and no more than two of X¹, X², X³ and X⁵ may be nitrogen,
wherein when X¹ is absent, no more than two of X², X³, X⁴ and X⁵ may be nitrogen and no more than one of X², X³ and X⁵ may be oxygen or sulfur;
R¹ is selected from the group comprising: C₁ or C₂ alkyl, C₁ or C₂ haloalkyl, and C₁ or C₂ alkylene-R^{1a}; wherein R^{1a} is selected from OR⁶, SR⁶, NR⁶R⁷, CO₂R⁶ and CONR⁶R⁶;
R² is selected from phenyl, 5- or 6-membered heteroaryl, 5- or 6-membered heterocycloalkyl or C₅ or C₆ cycloalkyl, and said phenyl, heteroaryl or cycloalkyl is optionally fused to or substituted with a group selected from phenyl, 5- or 6-membered heteroaryl, 5- or 6-membered heterocycloalkyl or C₅ or C₆ cycloalkyl; wherein any said phenyl or heteroaryl group is optionally substituted with at least one R⁸ group; or wherein any said heterocycloalkyl or cycloalkyl is optionally substituted with at least one R⁹ group;
R³, R⁶ and R¹¹ are each independently at each occurrence selected from the group comprising: H and C₁-C₆-alkyl;
R⁴ is independently at each occurrence selected from the group comprising: halo, C₁-C₆-alkyl, C₁-C₆-haloalkyl, C₁-C₆-alkylene-R¹⁰, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₃₋₆ cycloalkyl, C₂₋₆-alkenyl, C₂₋₆-alkynyl, phenyl and 5- or 6-membered heteroaryl;
R⁵ is selected from the group comprising: —C(O)NR⁶R¹⁴, —C(O)R¹², phenyl, 5- or 6-membered heteroaryl; 5-, 6-, 7-, 8-, 9- or 10-membered heterocycloalkyl and cyclopropyl; wherein any said phenyl or heteroaryl group is optionally substituted with at least one R⁸ group; or wherein any said heterocycloalkyl or cyclopropyl is optionally substituted with at least one R⁹ group;
wherein when X⁴ is nitrogen, R⁵ is selected such that R⁵ is attached to X⁴ via a carbon atom;
R⁷ is independently at each occurrence selected from the group comprising: H, C₁-C₆-alkyl, C(O)—C₁-C₆-alkyl and S(O)₂—C₁-C₆-alkyl;
R⁸ is independently at each occurrence selected from the group comprising: halo, C₁-C₆-alkyl, C₁-C₆-haloalkyl, C₁-C₆-alkylene-R¹⁰, —OR¹⁰, cyano, nitro, —NR⁶R⁷, —SR¹⁰, C(O)R¹⁰, C(O)OR¹⁰, C(O)NR⁶R¹⁰, —S(O)R¹⁰, —S(O)₂R¹⁰, —S(O)₂NR⁶R¹⁰, C₃₋₆ cycloalkyl, C₂₋₆-alkenyl, C₂₋₆-alkynyl, 5-, 6-, 7-, 8-, 9- or 10-membered heterocycloalkyl, phenyl and 5- or 6-membered heteroaryl; wherein R⁸ is optionally substituted where chemically possible with one or more R^{8c} groups;
R^{8c} is independently selected at each occurrence from: halo, C₁-C₆-alkyl, C₁-C₆-haloalkyl, C₁-C₆-alkylene-R¹⁰, C₁-C₆-alkylene-NR⁶R¹⁰, —OR¹⁰, C(O)R¹⁰, C(O)OR¹⁰, C(O)NR⁶R¹⁰;
R⁹ is independently at each occurrence selected from the group comprising: ═O, ═S, halo, C₁-C₆-alkyl, C₁-C₆-haloalkyl, —OR¹⁰, cyano, nitro, —NR⁶R⁷, —NR¹¹R¹², —SR¹⁰, C(O)R¹⁰, C(O)OR¹⁰, C(O)NR⁶R¹⁰, —S(O)R¹⁰, —S(O)₂R¹⁰, —S(O)₂NR⁶R¹⁰, C₃₋₆ cycloalkyl, 4-, 5- or 6-membered heterocycloalkyl, C₂₋₆-alkenyl C₂₋₆-alkynyl and C₁-C₃-alkylene-R^{9a}; wherein R^{9a} is selected from OR⁶, SR⁶, S(O)₂R⁶, S(O)₂NR⁶R⁶, S(O)₂Ph, NR⁶R⁷, CO₂R⁶, CONR⁶R⁶, 4-, 5- or 6-membered heterocycloalkyl, and cyclopropyl;
R¹⁰ is independently selected at each occurrence from the group comprising: H, C₁-C₆-alkyl, C₁-C₆-haloalkyl, C₁-C₆-alkylene-R¹⁰, C₃₋₈ cycloalkyl, 4-, 5-, 6-, 7- or 8-membered heterocycloalkyl, phenyl and 5- or 6-membered heteroaryl; wherein R^{10a} is independently selected at each occurrence from C₃₋₈ cycloalkyl, OR⁶, SR⁶, S(O)₂R⁶, S(O)₂Ph, NR⁶R⁷, CO₂R⁶ and CONR⁶R⁶;
R¹² is 6-membered heterocycloalkyl; wherein said heterocycloalkyl is optionally substituted with at least one R¹³ group;
R¹³ is independently at each occurrence selected from: ═O, ═S, halo, C₁-C₆-alkyl, C₁-C₆-haloalkyl, —OR⁶, cyano, nitro, —NR⁶R⁷, —SR⁶, C(O)R⁶, C(O)OR⁶, C(O)NR⁶R⁶, —S(O)R⁶, —S(O)₂R⁶, —S(O)₂NR⁶R⁶, C₃₋₆ cycloalkyl, C₂₋₆-alkenyl, C₂₋₆-alkynyl and C₁-C₃-alkylene-R^{13a}; wherein R^{13a} is selected from OR⁶, SR⁶, S(O)₂R⁶, S(O)₂Ph, NR⁶R⁷, CO₂R⁶ and CONR⁶R⁶;
R¹⁴ is selected from H and C₁-C₃-alkylene-R^{14a}; wherein R^{14a} is selected from OR⁶, SR⁶, S(O)₂R⁶, S(O)₂Ph, NR⁶R⁷, CO₂R⁶ and CONR⁶R⁶; and
n is an integer selected from 0, 1, 2, 3 or 4;
wherein any aforementioned alkyl, alkenyl, alkynyl, haloalkyl, cycloalkyl, heterocycloalkyl, phenyl, heteroaryl, alkylene, alkenylene, alkynylene, C(O)-alkyl and S(O)₂-alkyl is optionally substituted, where chemically possible, by 1 to 4 substituents which are each independently selected at each occurrence from the group consisting of: ═O; ═NR^{a}, ═NOR^{a}, C₁-C₄-alkyl, halo, nitro, cyano, C₁-C₄-haloalkyl, C₂-C₄-alkenyl, C₂-C₄-alkynyl, NR^{a}R^{b}, S(O)₂R^{a}, S(O)R^{a}, S(O)(NR^{a})R^{a}, S(O)₂NR^{a}R^{a}, CO₂R^{a}, C(O)R^{a}, CONR^{a}R^{a}, OR^{a} and SR^{a};
wherein R^{a} is independently selected from H and C₁-C₄-alkyl; and R^{b} is independently selected from H, C₁-C₄-alkyl, C(O)—C₁-C₄-alkyl and S(O)₂—C₁-C₄-alkyl.

In an embodiment of the first aspect, there is provided a compound of formula (Ia) or pharmaceutically acceptable salt thereof:

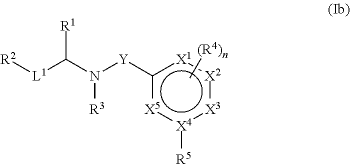

wherein
Y is —C(O)—, —C(S)—, —C(═NR⁶)—;
-L¹- is absent or a linker selected from C₁ alkylene, C₂-alkenylene, or C₂-alkynylene;
X¹ is absent or is selected from carbon and nitrogen;
X², X³ and X⁵ are each independently selected from carbon, nitrogen, oxygen and sulfur;
X⁴ is selected from carbon and nitrogen;
wherein when X¹ is carbon or nitrogen, X⁴ is carbon, X², X³ and X⁵ are each independently selected from carbon and nitrogen, and no more than two of X¹, X², X³ and X⁵ may be nitrogen,
wherein when X¹ is absent, no more than two of X², X³, X⁴ and X⁵ may be nitrogen and no more than one of X², X³ and X⁵ may be oxygen or sulfur;
R¹ is selected from the group comprising: C₁ or C₂ alkyl, C₁ or C₂ haloalkyl, and C₁ or C₂ alkylene-R^{1a}; wherein R^{1a} is selected from OR⁶, SR⁶, NR⁶R⁷, CO₂R⁶ and CONR⁶R⁶;
R² is selected from phenyl, 5- or 6-membered heteroaryl, 5- or 6-membered heterocycloalkyl or C₅ or C₆ cycloalkyl, and said phenyl, heteroaryl or cycloalkyl is optionally fused to a group selected from phenyl, 5- or 6-membered heteroaryl, 5- or 6-membered heterocycloalkyl or C₅ or C₆ cycloalkyl; wherein any said phenyl or heteroaryl group is optionally substituted with at least one R⁸ group; or wherein any said heterocycloalkyl or cycloalkyl is optionally substituted with at least one R⁹ group;
R³, R⁶ and R¹¹ are each independently at each occurrence selected from the group comprising: H and C₁-C₆-alkyl;
R⁴ and R⁸ are each independently at each occurrence selected from the group comprising: halo, C₁-C₆-alkyl, C₁-C₆-haloalkyl, C₁-C₆-alkylene-R¹⁰, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₃₋₆ cycloalkyl, C₂₋₆-alkenyl, C₂₋₆-alkynyl, phenyl and 5- or 6-membered heteroaryl;
R⁵ is selected from the group comprising: —C(O)NR⁶R¹⁴, —C(O)R¹², phenyl, 6-membered heteroaryl, 5-, 6- or 7- or 8-membered heterocycloalkyl and cycopropyl; wherein any said phenyl or heteroaryl group is optionally substituted with at least one R⁸ group; or wherein any said heterocycloalkyl or cyclopropyl is optionally substituted with at least one R⁹ group;
wherein when X⁴ is nitrogen, R⁵ is selected such that R⁵ is attached to X⁴ via a carbon atom;
R⁷ is independently at each occurrence selected from the group comprising: H, C₁-C₆-alkyl, C(O)—C₁-C₆-alkyl and S(O)₂—C₁-C₆-alkyl;
R⁹ is independently at each occurrence selected from the group comprising: ═O, ═S, halo, C₁-C₆-alkyl, C₁-C₆-haloalkyl, —OR⁶, cyano, nitro, —NR⁶R⁷, —NR¹¹R¹², —SR⁶, C(O)R⁶, C(O)OR⁶, C(O)NR⁶R⁶, —S(O)R⁶, —S(O)₂R⁶, —S(O)₂NR⁶R⁶, C₃₋₆ cycloalkyl, 4-, 5- or 6-membered heterocycloalkyl, C₂₋₆-alkenyl C₂₋₆-alkynyl and C₁-C₆-alkylene-R^{9a}; wherein R^{9a} is selected from OR⁶, SR⁶, S(O)₂R⁶, S(O)₂NR⁶R⁶, S(O)₂Ph, NR⁶R⁷, CO₂R⁶, CONR⁶R⁶, and cyclopropyl;
R¹⁰ is independently selected at each occurrence from the group comprising: H, C₁-C₆-alkyl, C₁-C₆-haloalkyl, C₁-C₆-alkylene-R¹⁰, C₃₋₈ cycloalkyl, and 4-, 5-, 6-, 7- or 8-membered heterocycloalkyl; wherein R^{10a} is independently selected at each occurrence from C₃₋₈ cycloalkyl, OR⁶, SR⁶, S(O)₂R⁶, S(O)₂Ph, NR⁶R⁷, CO₂R⁶ and CONR⁶R⁶;
R¹² is 6-membered heterocycloalkyl; wherein said heterocycloalkyl is optionally substituted with at least one R¹³ group;
R¹³ is independently at each occurrence selected from: ═O, ═S, halo, C₁-C₆-alkyl, C₁-C₆-haloalkyl, —OR⁶, cyano, nitro, —NR⁶R⁷, —SR⁶, C(O)R⁶, C(O)OR⁶, C(O)NR⁶R⁶, —S(O)R⁶, —S(O)₂R⁶, —S(O)₂NR⁶R⁶, C₃₋₆ cycloalkyl, C₂₋₆-alkenyl, C₂₋₆-alkynyl and C₁-C₆-alkylene-R^{13a}; wherein R^{13a} is selected from OR⁶, SR⁶, S(O)₂R⁶, S(O)₂Ph, NR⁶R⁷, CO₂R⁶ and CONR⁶R⁶;
R¹⁴ is selected from H and C₁-C₆-alkylene-R^{14a}; wherein R^{14a} is selected from OR⁶, SR⁶, S(O)₂R⁶, S(O)₂Ph, NR⁶R⁷, CO₂R⁶ and CONR⁶R⁶; and
n is an integer selected from 0, 1, 2, 3 or 4;
wherein any aforementioned alkyl, alkenyl, alkynyl, haloalkyl, cycloalkyl, heterocycloalkyl, phenyl, heteroaryl, alkylene, alkenylene, alkynylene, C(O)-alkyl and S(O)₂-alkyl is optionally substituted, where chemically possible, by 1 to 4 substituents which are each independently selected at each occurrence from the group consisting of: ═O; ═NR^{a}, ═NOR^{a}, C₁-C₄-alkyl, halo, nitro, cyano, C₁-C₄-haloalkyl, C₂-C₄-alkenyl, C₂-C₄-alkynyl, NR^{a}R^{b}, S(O)₂R^{a}, S(O)R^{a}, S(O)(NR^{a})R^{a}, S(O)₂NR^{a}R^{a}, CO₂R^{a}, C(O)R^{a}, CONR^{a}R^{a}, OR^{a} and SR^{a}; wherein R^{a} is independently selected from H and C₁-C₄-alkyl; and R^{b} is independently selected from H, C₁-C₄-alkyl, C(O)—C₁-C₄-alkyl and S(O)₂—C₁-C₄-alkyl.

In certain embodiments, the compound of formula (I), formula (Ia) or formula (Ib) is a compound of formula (II):

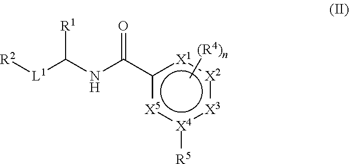

wherein L¹, X¹, X², X³, X⁴, X⁵, R¹, R², R⁴, R⁵ and n are as described above for formula (I), formula (Ia) or formula (Ib).

In certain embodiments, the compound of formula (I), formula (Ia) or formula (Ib) is a compound of formula (III):

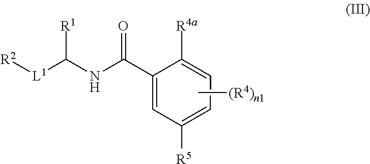

wherein L¹, R¹, R², R⁴ and R⁵ are as described above for formula (I) formula (Ia), or formula (Ib) and wherein R^{4a} is independently selected from the group comprising: halo, C₁-C₆-alkyl, C₁-C₆-haloalkyl, C₁-C₆-alkylene-R¹⁰, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₃₋₆ cycloalkyl, C₂₋₆-alkenyl, C₂₋₆-alkynyl, phenyl and 5- or 6-membered heteroaryl; and n1 is an integer selected from 0, 1, 2 or 3.

In certain embodiments, the compound of formula (I), formula (Ia) or formula (Ib) is a compound of formula (IV):

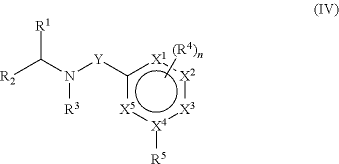

wherein Y, X¹, X², X³, X⁴, X⁵, R¹, R², R³, R⁴, R⁵ and n are as described above for formula (I), formula (Ia) or formula (Ib).

In certain embodiments, the compound of formula (I), formula (Ia) or formula (Ib) is a compound of formula (V):

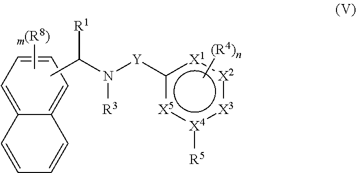

wherein Y, X¹, X², X³, X⁴, X⁵, R¹, R³, R⁴, R⁵, R⁸ and n are as described above for formula (I), formula (Ia) or formula (Ib); and wherein m is an integer independently selected from 0, 1, 2, 3, 4, 5, 6, and 7.

In certain embodiments, the compound of formula (I), formula (Ia) or formula (Ib) is a compound of formula (VI):

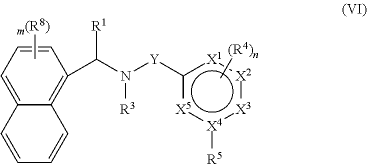

wherein Y, X¹, X², X³, X⁴, X⁵, R¹, R³, R⁴, R⁵, R⁸ and n are as described above for formula (I), formula (Ia) or formula (Ib); and wherein m is an integer independently selected from 0, 1, 2, 3, 4, 5, 6, and 7.

In certain embodiments, the compound of formula (I), formula (Ia) or formula (Ib) is a compound of formula (VII):

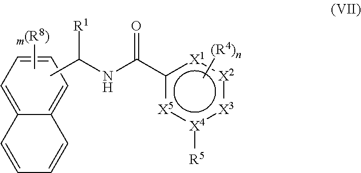

wherein X¹, X², X³, X⁴, X⁵, R¹, R⁴, R⁵, R⁸ and n are as described above for formula (I), formula (Ia) or formula (Ib) and wherein m is an integer independently selected from 0, 1, 2, 3, 4, 5, 6, and 7.

In certain embodiments, the compound of formula (I), formula (Ia) or formula (Ib) is a compound of formula (VIII):

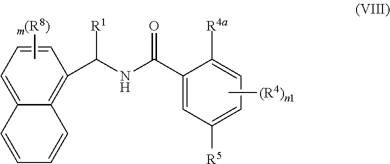

wherein R¹, R⁴, R⁵ and R⁸ are as described above for formula (I), formula (Ia) or formula (Ib); and wherein R⁴ is independently selected from the group comprising: halo, C₁-C₆-alkyl, C₁-C₆-haloalkyl, C₁-C₆-alkylene-R¹⁰, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₃₋₆ cycloalkyl, C₂₋₆-alkenyl, C₂₋₆-alkynyl, phenyl and 5- or 6-membered heteroaryl; n1 is an integer selected from 0, 1, 2 or 3; and m is an integer independently selected from 0, 1, 2, 3, 4, 5, 6, and 7.

In certain embodiments, the compound of formula (I) or formula (Ia) is a compound of formula (IX):

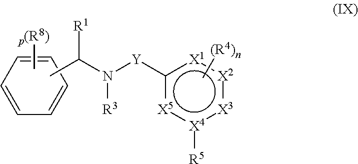

wherein Y, X¹, X², X³, X⁴, X⁵, R¹, R³, R⁴, R⁵, R⁸ and n are as described above for formula (I) or formula (Ia); and wherein p is an integer independently selected from 0, 1, 2, 3, 4, and 5.

In certain embodiments, the compound of formula (I) or formula (Ia) is a compound of formula (X):

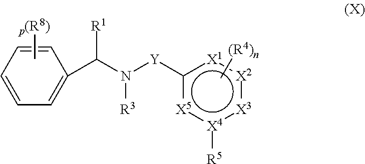

wherein Y, X¹, X², X³, X⁴, X⁵, R¹, R³, R⁴, R⁵, R⁸ and n are as described above for formula (I) or formula (Ia); and wherein p is an integer independently selected from 0, 1, 2, 3, 4, and 5.

In certain embodiments, the compound of formula (I) or formula (Ia) is a compound of formula (XI):

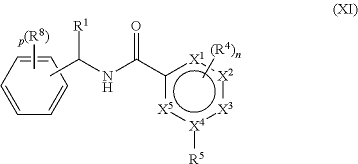

wherein X¹, X², X³, X⁴, X⁵, R¹, R⁴, R⁵, R⁸ and n are as described above for formula (I) or formula (Ia) and wherein p is an integer independently selected from 0, 1, 2, 3, 4, and 5.

In certain embodiments, the compound of formula (I) or formula (Ia) is a compound of formula (XII):

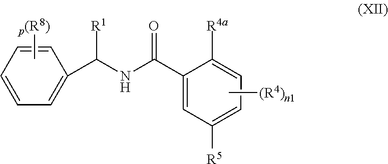

wherein R¹, R⁴, R⁵ and R⁸ are as described above for formula (I) or formula (Ia); and wherein R^{4a} is independently selected from the group comprising: halo, C₁-C₆-alkyl, C₁-C₆-haloalkyl, C₁-C₆-alkylene-R¹⁰, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₃₋₆ cycloalkyl, C₂₋₆-alkenyl, C₂₋₆-alkynyl, phenyl and 5- or 6-membered heteroaryl; n1 is an integer selected from 0, 1, 2 or 3; and p is an integer independently selected from 0, 1, 2, 3, 4, and 5.

In certain embodiments, the compound of formula (I) or formula (Ia) is a compound of formula (XIII):

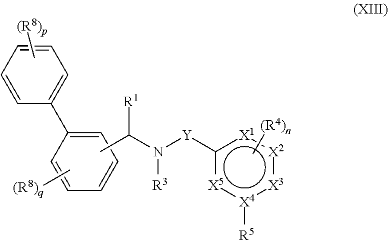

wherein Y, X¹, X², X³, X⁴, X⁵, R¹, R³, R⁴, R⁵, R⁸ and n are as described above for formula (I) or formula (Ia); wherein p is an integer independently selected from 0, 1, 2, 3, 4, and 5; and wherein q is an integer independently selected from 0, 1, 2, 3, and 4.

In certain embodiments, the compound of formula (I) or formula (Ia) is a compound of formula (XIV):

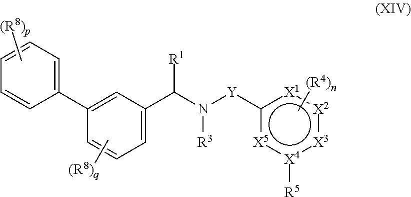

wherein Y, X¹, X², X³, X⁴, X⁵, R¹, R³, R⁴, R⁵, R⁸ and n are as described above for formula (I) or formula (Ia); wherein p is an integer independently selected from 0, 1, 2, 3, 4, and 5; and wherein q is an integer independently selected from 0, 1, 2, 3, and 4.

In certain embodiments, the compound of formula (I) or formula (Ia) is a compound of formula (XV):

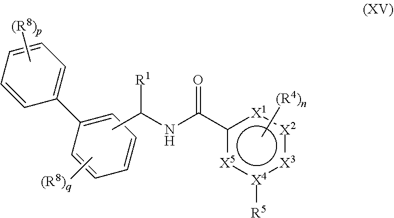

wherein X¹, X², X³, X⁴, X⁵, R¹, R⁴, R⁵, R⁸ and n are as described above for formula (I) or formula (Ia); wherein p is an integer independently selected from 0, 1, 2, 3, 4, and 5; and wherein q is an integer independently selected from 0, 1, 2, 3, and 4.

In certain embodiments, the compound of formula (I) or formula (Ia) is a compound of formula (XVI):

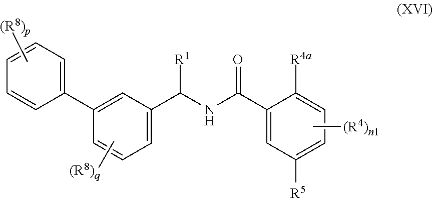

wherein R¹, R⁴, R⁵ and R⁸ are as described above for formula (I) or formula (Ia); and wherein R⁴ is independently selected from the group comprising: halo, C₁-C₆-alkyl, C₁-C₆-haloalkyl, C₁-C₆-alkylene-R¹⁰, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₃₋₆ cycloalkyl, C₂₋₆-alkenyl, C₂₋₆-alkynyl, phenyl and 5- or 6-membered heteroaryl; n1 is an integer selected from 0, 1, 2 or 3; wherein p is an integer independently selected from 0, 1, 2, 3, 4, and 5; and wherein q is an integer independently selected from 0, 1, 2, 3, and 4.

In certain embodiments, the compound of formula (I), formula (Ia) or formula (Ib) is a compound of formula (XVII):

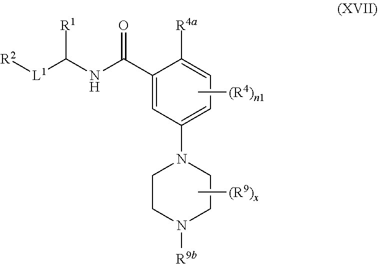

wherein L¹, R¹, R², R⁴ and R⁵ are as described above for formula (I), formula (Ia) or formula (Ib) and wherein R⁴ is independently selected from the group comprising: halo, C₁-C₆-alkyl, C₁-C₆-haloalkyl, C₁-C₆-alkylene-R¹⁰, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₃₋₆ cycloalkyl, C₂₋₆-alkenyl, C₂₋₆-alkynyl, phenyl and 5- or 6-membered heteroaryl; R^{9b} is independently at each occurrence selected from the group comprising: H, C₁-C₆-alkyl, C₁-C₆-haloalkyl, C(O)R⁶, C(O)OR⁶, C(O)NR⁶R⁶, —S(O)R⁶, —S(O)₂R⁶, —S(O)₂NR⁶R⁶, C₃₋₆ cycloalkyl, 4-, 5- or 6-membered heterocycloalkyl, C₂₋₆-alkenyl C₂₋₆-alkynyl, C₂-C₃-alkylene-R^{9a}, and CH₂-cyclopropyl; n1 is an integer selected from 0, 1, 2 or 3; and x is an integer selected from 0, 1, 2, 3, 4, 5 and 6.

In certain embodiments, the compound of formula (I), formula (Ia) or formula (Ib) is a compound of formula (XVIII):

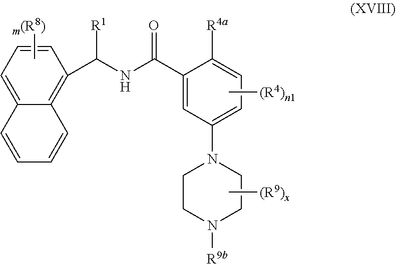

wherein R¹, R⁴, R⁵ and R⁸ are as described above for formula (I), formula (Ia) or formula (Ib); and wherein R⁴ is independently selected from the group comprising: halo, C₁-C₆-alkyl, C₁-C₆-haloalkyl, C₁-C₆-alkylene-R¹⁰, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₃₋₆ cycloalkyl, C₂₋₆-alkenyl, C₂₋₆-alkynyl, phenyl and 5- or 6-membered heteroaryl; R^{9b} is independently at each occurrence selected from the group comprising: C₁-C₆-alkyl, C₁-C₆-haloalkyl, C(O)R⁶, C(O)OR⁶, C(O)NR⁶R⁶, —S(O)R⁶, —S(O)₂R⁶, —S(O)₂NR⁶R⁶, C₃₋₆ cycloalkyl, 4-, 5- or 6-membered heterocycloalkyl, C₂₋₆-alkenyl C₂₋₆-alkynyl, C₂-C₃-alkylene-R^{9a} and CH₂-cyclopropyl; n1 is an integer selected from 0, 1, 2 or 3; and m is an integer independently selected from 0, 1, 2, 3, 4, 5, 6, and 7; and x is an integer selected from 0, 1, 2, 3, 4, 5 and 6.

In certain embodiments, the compound of formula (I) or formula (Ia) is a compound of formula (XIX):

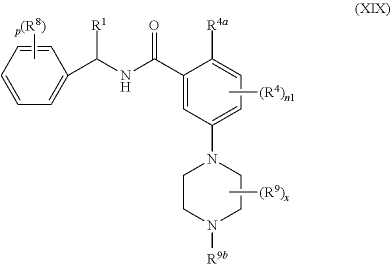

wherein R¹, R⁴, R⁵ and R⁸ are as described above for formula (I) or formula (Ia); and wherein R⁴ is independently selected from the group comprising: halo, C₁-C₆-alkyl, C₁-C₆-haloalkyl, C₁-C₆-alkylene-R¹⁰, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₃₋₆ cycloalkyl, C₂₋₆-alkenyl, C₂₋₆-alkynyl, phenyl and 5- or 6-membered heteroaryl; R^{9b} is independently at each occurrence selected from the group comprising: C₁-C₆-alkyl, C₁-C₆-haloalkyl, C(O)R¹⁰, C(O)OR¹⁰, C(O)NR⁶R¹⁰, —S(O)R¹⁰, —S(O)₂R¹⁰, —S(O)₂NR⁶R¹⁰, C₃₋₆ cycloalkyl, 4-, 5- or 6-membered heterocycloalkyl, C₂₋₆-alkenyl C₂₋₆-alkynyl, C₂-C₃-alkylene-R^{9a} and CH₂-cyclopropyl; n1 is an integer selected from 0, 1, 2 or 3; and p is an integer independently selected from 0, 1, 2, 3, 4, and 5; and x is an integer selected from 0, 1, 2, 3, 4, 5 and 6.

In certain embodiments, the compound of formula (I) or formula (Ia) is a compound of formula (XX):

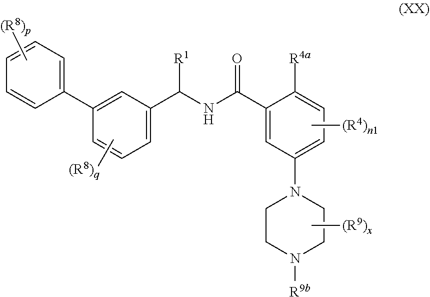

wherein R¹, R⁴, R⁵ and R⁸ are as described above for formula (I) or formula (Ia); and wherein R⁴ is independently selected from the group comprising: halo, C₁-C₆-alkyl, C₁-C₆-haloalkyl, C₁-C₆-alkylene-R¹⁰, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₃₋₆ cycloalkyl, C₂₋₆-alkenyl, C₂₋₆-alkynyl, phenyl and 5- or 6-membered heteroaryl; R^{9b} is independently at each occurrence selected from the group comprising: C₁-C₆-alkyl, C₁-C₆-haloalkyl, C(O)R¹⁰, C(O)OR¹⁰, C(O)NR⁶R¹⁰, —S(O)R¹⁰, —S(O)₂R¹⁰, —S(O)₂NR⁶R¹⁰, C₃₋₆ cycloalkyl, 4-, 5- or 6-membered heterocycloalkyl, C₂₋₆-alkenyl C₂₋₆-alkynyl, C₂-C₃-alkylene-R^{9a} and CH₂-cyclopropyl; n1 is an integer selected from 0, 1, 2 or 3; p is an integer independently selected from 0, 1, 2, 3, 4, and 5; q is an integer independently selected from 0, 1, 2, 3 or 4; and x is an integer selected from 0, 1, 2, 3, 4, 5 and 6.

In certain embodiments, the compound of formula (I) or formula (Ia) is a compound of formula (XXI):

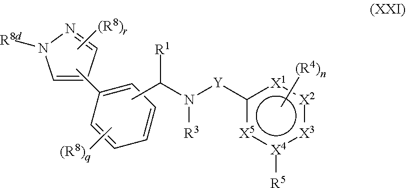

wherein Y, X¹, X², X³, X⁴, X⁵, R¹, R³, R⁴, R⁵, R⁸ and n are as described above for formula (I) or formula (Ia); wherein R^{d} is independently selected from H, halo, C₁-C₆-alkyl, C₁-C₆-haloalkyl, C₁-C₆-alkylene-R¹⁰, —OR¹⁰, cyano, nitro, —NR⁶R⁷, —SR¹⁰, C(O)R¹⁰, C(O)OR¹⁰, C(O)NR⁶R¹⁰, —S(O)R¹⁰, —S(O)₂R¹⁰, —S(O)₂NR⁶R¹⁰, C₃₋₆ cycloalkyl, C₂₋₆-alkenyl, C₂₋₆-alkynyl, 5-, 6-, 7-, 8-, 9- or 10-membered heterocycloalkyl, phenyl and 5- or 6-membered heteroaryl; wherein q is an integer independently selected from 0, 1, 2, 3, and 4; and wherein r is an integer independently selected from 0, 1 and 2.

In certain embodiments, the compound of formula (I) or formula (Ia) is a compound of formula (XXII):

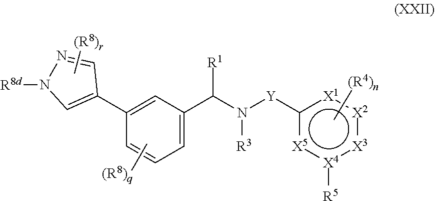

wherein Y, X¹, X², X³, X⁴, X⁵, R¹, R³, R⁴, R⁵, R⁸ and n are as described above for formula (I) or formula (Ia); wherein R^{8d} is as described above for formula (XXI); wherein q is an integer independently selected from 0, 1, 2, 3, and 4; and wherein r is an integer independently selected from 0, 1 and 2.

In certain embodiments, the compound of formula (I) or formula (Ia) is a compound of formula (XXIII):

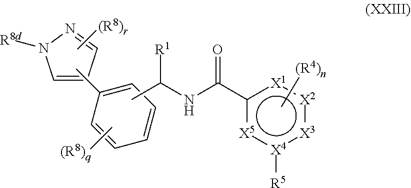

wherein X¹, X², X³, X⁴, X⁵, R¹, R⁴, R⁵, R⁸ and n are as described above for formula (I) or formula (Ia); wherein R^{8d} is as described above for formula (XXI); wherein q is an integer independently selected from 0, 1, 2, 3, and 4; and wherein r is an integer independently selected from 0, 1 and 2.

In certain embodiments, the compound of formula (I) or formula (Ia) is a compound of formula (XXIV):

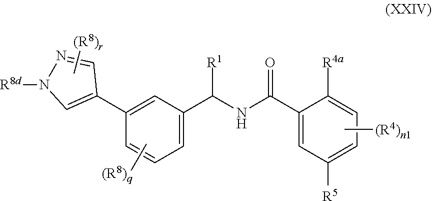

wherein R¹, R⁴, R⁵ and R⁸ are as described above for formula (I) or formula (Ia); wherein R^{8d} is as described above for formula (XXI); and wherein R^{4a} is independently selected from the group comprising: halo, C₁-C₆-alkyl, C₁-C₆-haloalkyl, C₁-C₆-alkylene-R¹⁰, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₃₋₆ cycloalkyl, C₂₋₆-alkenyl, C₂₋₆-alkynyl, phenyl and 5- or 6-membered heteroaryl; n1 is an integer selected from 0, 1, 2 or 3; wherein q is an integer independently selected from 0, 1, 2, 3, and 4; and wherein r is an integer independently selected from 0, 1 and 2.

The following embodiments apply to compounds of any of formulae (I), (Ia), (Ib) and (II)-(XXIV). These embodiments are independent and interchangeable. Any one embodiment may be combined with any other embodiment, where chemically allowed. In other words, any of the features described in the following embodiments may (where chemically allowable) be combined with the features described in one or more other embodiments. In particular, where a compound is exemplified or illustrated in this specification, any two or more of the embodiments listed below, expressed at any level of generality, which encompass that compound may be combined to provide a further embodiment which forms part of the present disclosure.

It may be that Y is —C(O)—. It may be that Y is —C(S)—. It may be that Y is —C(═NR⁶)—.
-L¹- may be absent, —CH₂—, —CH₂CH₂— or —CHCH—. -L¹- may be absent or —CH₂—. -L¹- may be —CH₂—, —CH₂CH₂— or —CHCH—. Preferably, -L¹- is absent.

It may that Y is —C(O)— and -L¹- is absent.

X¹ may be selected from carbon and nitrogen. In these embodiments, X⁴ is carbon, X², X³ and X⁵ are each independently selected from carbon and nitrogen, and no more than two of X¹, X², X³ and X⁵ may be nitrogen. It may be that each of X¹, X², X³, and X⁵ is carbon. It may be that at least one of X¹, X², X³ and X⁵ is nitrogen. It may be that a single one of X¹, X², X³ and X⁵ is nitrogen. It may be that X¹ is carbon. It may be that at least one of X², X³ and X⁵ is nitrogen. It may be that a single one of X², X³ and X⁵ is nitrogen. It may be that X¹ is nitrogen and each of X², X³, and X⁵ is carbon. It may be that X⁵ is nitrogen and each of X¹, X², and X³ is carbon. It may be that each of X², X³ and X⁴ is carbon.

The ring comprising X¹, X², X³, X⁴, and X⁵ may be:

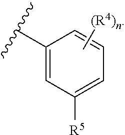

The ring comprising X¹, X², X³, X⁴, and X⁵ may be:

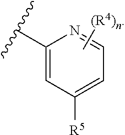

The ring comprising X¹, X², X³, X⁴, and X⁵ may be:

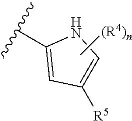

The ring comprising X¹, X², X³, X⁴, and X⁵ may be:

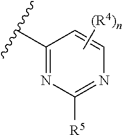

The ring comprising X¹, X², X³, X⁴, and X⁵ may be:

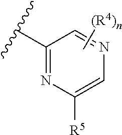

The ring comprising X¹, X², X³, X⁴, and X⁵ may be:

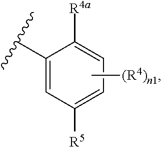

wherein R^{4a} is independently selected from the group comprising: halo, C₁-C₆-alkyl, C₁-C₆-haloalkyl, C₁-C₆-alkylene-R¹⁰, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₃₋₆ cycloalkyl, C₂₋₆-alkenyl, C₂₋₆-alkynyl, phenyl and 5- or 6-membered heteroaryl; and n1 is an integer selected from 0, 1, 2 or 3.

It may be that X¹ is absent. In these embodiments, no more than two of X², X³, X⁴ and X⁵ may be nitrogen and no more than one of X², X³ and X⁵ may be oxygen or sulfur.

R¹ may be C₁ or C₂ alkyl, e.g. methyl or ethyl. R¹ may be C₁ or C₂ haloalkyl, e.g. CF₃, CH₂CF₃, CH(CF₃)CH₃. R¹ may be C₁ or C₂ alkylene-R^{1a}, wherein R^{1a} is selected from OR⁶, SR⁶, NR⁶R⁷, CO₂R⁶ and CONR⁶R⁶, e.g. CH₂—R^{1a} or CH₂CH₂R^{1a}. Preferably, R¹ is methyl.

It may be that -L¹- is absent and R¹ is C₁ or C₂ alkyl. It may be that L¹ is absent and R¹ is methyl.

R² may be selected from phenyl, 5- or 6-membered heteroaryl, 5- or 6-membered heterocycloalkyl or C₅ or C₆ cycloalkyl, and said phenyl, heteroaryl or cycloalkyl is optionally fused to a group selected from phenyl, 5- or 6-membered heteroaryl, 5- or 6-membered heterocycloalkyl or C₅ or C₆ cycloalkyl; wherein any said phenyl or heteroaryl group is optionally substituted with at least one R⁸ group; or wherein any said heterocycloalkyl or cycloalkyl is optionally substituted with at least one R⁹ group.

It may be that R² is selected from phenyl, 5- or 6-membered heteroaryl; where said phenyl or heteroaryl is optionally fused to a group selected from phenyl, 5- or 6-membered heteroaryl, 5- or 6-membered heterocycloalkyl or C₅ or C₆ cycloalkyl; wherein any said phenyl or heteroaryl group is optionally substituted with at least one R⁸ group; or wherein any said heterocycloalkyl or cycloalkyl is optionally substituted with at least one R⁹ group.

It may be that R² is selected from phenyl, 5- or 6-membered heteroaryl; where said phenyl or heteroaryl is optionally fused to or substituted with a group selected from phenyl, 5- or 6-membered heteroaryl, 5- or 6-membered heterocycloalkyl or C₅ or C₆ cycloalkyl; wherein any said phenyl or heteroaryl group is optionally substituted with at least one R⁸ group; or wherein any said heterocycloalkyl or cycloalkyl is optionally substituted with at least one R⁹ group.

It may be that R² is selected from phenyl, 5- or 6-membered heteroaryl; where said phenyl or heteroaryl is optionally fused to or substituted with a group selected from phenyl, and 5- or 6-membered heteroaryl; wherein any said phenyl or heteroaryl group is optionally substituted with at least one R⁸ group.

It may be that R² is selected from the group comprising phenyl, pyridyl, naphthyl, indolyl, benzofuryl, benzothiophenyl, and quinolinyl. It may be that R² is phenyl or naphthyl. R² may be naphthyl, e.g. naphth-2-yl.

It may be that R² is selected from the group comprising phenyl, biphenyl, phenylpyrrolyl, phenylthiophenyl, pyridyl, naphthyl, indolyl, benzofuryl, benzothiophenyl, and quinolinyl. It may be that R² is phenyl, biphenyl, phenylpyrrolyl, phenylthiophenyl or naphthyl. It may be that R² is phenyl, biphenyl or naphthyl. R² may be naphthyl, e.g. naphth-2-yl. R² may be phenyl. R² may be biphenyl.

It may be that R² has the structure:

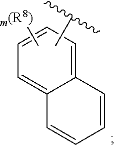

wherein m is an integer independently selected from 0, 1, 2, 3, 4, 5, 6, and 7.

It may be that R² has the structure:

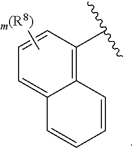

It may be that R² has the structure:

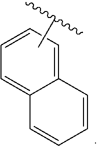

It may be that R² has the structure:

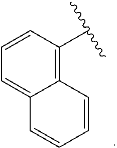 [SMILES: *c1cccc2ccccc12]

It may be that R² has the structure:

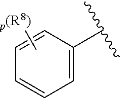

wherein p is an integer selected from 0, 1, 2, 3, 4 and 5. In these embodiments, it may be that R⁸ is independently selected at each occurrence from the group comprising: halo, C₁-C₆-alkyl, C₁-C₆-haloalkyl, C₁-C₆-alkylene-R¹⁰, —OR¹⁰, cyano, nitro, —NR⁶R⁷, —SR¹⁰, C(O)R¹⁰, C(O)OR¹⁰, C(O)NR⁶R¹⁰, —S(O)R¹⁰, —S(O)₂R¹⁰, —S(O)₂NR⁶R¹⁰, C₂₋₆-alkenyl, C₂₋₆-alkynyl, and 5- or 6-membered heterocycloalkyl. In these embodiments, it may be that R^{a} is independently selected at each occurrence from the group comprising: halo, C₁-C₆-alkyl, C₁-C₆-haloalkyl, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₂₋₆-alkenyl, C₂₋₆-alkynyl. R⁸ may be independently selected at each occurrence from the group comprising: halo, C₁-C₄-alkyl, C₁-C₄-haloalkyl, —OR¹⁰, cyano, and —NR⁶R⁷.

It may be that R² has the structure:

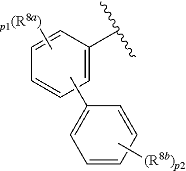

wherein R^{8a} is independently at each occurrence selected from halo, C₁-C₆-alkyl, C₁-C₆-haloalkyl, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₂₋₆-alkenyl, C₂₋₆-alkynyl; R^{8b} is independently at each occurrence selected from C₁-C₄-alkyl, halo, nitro, cyano, C₁-C₄-haloalkyl, C₂-C₄-alkenyl, C₂-C₄-alkynyl, NR^{a}R^{b}, S(O)₂R^{a}, S(O)R^{a}, S(O)(NR^{a})R^{a}, S(O)₂NR^{a}R^{a}, CO₂R^{a}, C(O)R^{a}, CONR^{a}R^{a}, OR^{a} and SR^{a}; p1 is an integer selected from 0, 1, 2, 3 and 4; and p2 is an integer selected from 0, 1, 2, 3, 4 and 5.

It may be that R² has the structure:

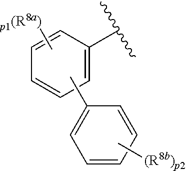

wherein R^{8a} is independently at each occurrence selected from halo, C₁-C₆-alkyl, C₁-C₆-haloalkyl, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₂₋₆-alkenyl, C₂₋₆-alkynyl; R^{8b} is independently at each occurrence selected from C₁-C₄-alkyl, C₁-C₄-alkylene-R¹⁰ halo, nitro, cyano, C₁-C₄-haloalkyl, C₂-C₄-alkenyl, C₂-C₄-alkynyl, NR⁶R⁷, S(O)₂R¹⁰, S(O)R¹⁰, S(O)₂NR⁶R¹⁰, CO₂R¹⁰, C(O)R¹⁰, CONR⁶R¹⁰, OR¹⁰, SR¹⁰, 5- or 6-membered heterocycloalkyl, phenyl and 5- or 6-membered heteroaryl; p1 is an integer selected from 0, 1, 2, 3 and 4; and p2 is an integer selected from 0, 1, 2, 3, 4 and 5.

It may be that R² has the structure:

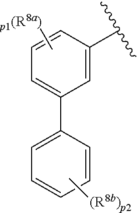

wherein R^{8a} is independently at each occurrence selected from halo, C₁-C₆-alkyl, C₁-C₆-haloalkyl, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₂₋₆-alkenyl, C₂₋₆-alkynyl; R^{8b} is independently at each occurrence selected from C₁-C₄-alkyl, C₁-C₄-alkylene-R¹⁰ halo, nitro, cyano, C₁-C₄-haloalkyl, C₂-C₄-alkenyl, C₂-C₄-alkynyl, NR⁶R⁷, S(O)₂R¹⁰, S(O)R¹⁰, S(O)₂NR⁶R¹⁰, CO₂R¹⁰, C(O)R¹⁰, CONR⁶R¹⁰, OR¹⁰, SR¹⁰, 5- or 6-membered heterocycloalkyl, phenyl and 5- or 6-membered heteroaryl; p1 is an integer selected from 0, 1, 2, 3 and 4; and p2 is an integer selected from 0, 1, 2, 3, 4 and 5.

It may be that R² has the structure:

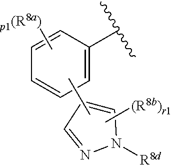

wherein R^{8a} is independently at each occurrence selected from halo, C₁-C₆-alkyl, C₁-C₆-haloalkyl, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₂₋₆-alkenyl, C₂₋₆-alkynyl; R^{8b} is independently at each occurrence selected from C₁-C₄-alkyl, halo, nitro, cyano, C₁-C₄-haloalkyl, C₂-C₄-alkenyl, C₂-C₄-alkynyl, NR^{a}R^{b}, S(O)₂R^{a}, S(O)R^{a}, S(O)(NR^{a})R^{a}, S(O)₂NR^{a}R^{a}, CO₂R^{a}, C(O)R^{a}, CONR^{a}R^{a}, OR^{a} and SR^{a}; R^{8d} is independently selected from H, halo, C₁-C₆-alkyl, C₁-C₆-haloalkyl, C₁-C₆-alkylene-R¹⁰, —OR¹⁰, cyano, nitro, —NR⁶R⁷, —SR¹⁰, C(O)R¹⁰, C(O)OR¹⁰, C(O)NR⁶R¹⁰, —S(O)R¹⁰, —S(O)₂R¹⁰, —S(O)₂NR⁶R¹⁰, C₃₋₆ cycloalkyl, C₂₋₆-alkenyl, C₂₋₆-alkynyl; p1 is an integer selected from 0, 1, 2, 3 and 4; and r1 is an integer selected from 0, 1 and 2.

It may be that R² has the structure:

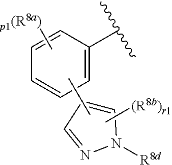

wherein R^{8a} is independently at each occurrence selected from halo, C₁-C₆-alkyl, C₁-C₆-haloalkyl, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₂₋₆-alkenyl, C₂₋₆-alkynyl; R^{8b} is independently at each occurrence selected from C₁-C₄-alkyl, C₁-C₄-alkylene-R¹⁰ halo, nitro, cyano, C₁-C₄-haloalkyl, C₂-C₄-alkenyl, C₂-C₄-alkynyl, NR⁶R⁷, S(O)₂R¹⁰, S(O)R¹⁰, S(O)₂NR⁶R¹⁰, CO₂R¹⁰, C(O)R¹⁰, CONR⁶R¹⁰, OR¹⁰, SR¹⁰, 5- or 6-membered heterocycloalkyl, phenyl and 5- or 6-membered heteroaryl; R^{8d} is independently selected from H, halo, C₁-C₆-alkyl, C₁-C₆-haloalkyl, C₁-C₆-alkylene-R¹⁰, —OR¹⁰, cyano, nitro, —NR⁶R⁷, —SR¹⁰, C(O)R¹⁰, C(O)OR¹⁰, C(O)NR⁶R¹⁰, —S(O)R¹⁰, —S(O)₂R¹⁰, —S(O)₂NR⁶R¹⁰, C₃₋₆ cycloalkyl, C₂₋₆-alkenyl, C₂₋₆-alkynyl, 5-, 6-, 7-, 8-, 9- or 10-membered heterocycloalkyl, phenyl and 5- or 6-membered heteroaryl; p1 is an integer selected from 0, 1, 2, 3 and 4; and r1 is an integer selected from 0, 1 and 2.

It may be that R² has the structure:

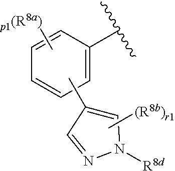

wherein R^{8a} is independently at each occurrence selected from halo, C₁-C₆-alkyl, C₁-C₄-haloalkyl, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₂₋₆-alkenyl, C₂₋₆-alkynyl; R^{8b} is independently at each occurrence selected from C₁-C₄-alkyl, halo, nitro, cyano, C₁-C₄-haloalkyl, C₂-C₄-alkenyl, C₂-C₄-alkynyl, NR^{a}R^{b}, S(O)₂R^{a}, S(O)R^{a}, S(O)(NR^{a})R^{a}, S(O)₂NR^{a}R^{a}, CO₂R^{a}, C(O)R^{a}, CONR^{a}R^{a}, OR^{a} and SR^{a}; R^{8d} is independently selected from H, halo, C₁-C₆-alkyl, C₁-C₆-haloalkyl, C₁-C₆-alkylene-R¹⁰, —OR¹⁰, cyano, nitro, —NR⁶R⁷, —SR¹⁰, C(O)R¹⁰, C(O)OR¹⁰, C(O)NR⁶R¹⁰, —S(O)R¹⁰, —S(O)₂R¹⁰, —S(O)₂NR⁶R¹⁰, C₃₋₆ cycloalkyl, C₂₋₆-alkenyl, C₂₋₆-alkynyl; p1 is an integer selected from 0, 1, 2, 3 and 4; and r1 is an integer selected from 0, 1 and 2.

It may be that R² has the structure:

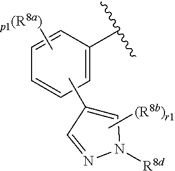

wherein R^{8a} is independently at each occurrence selected from halo, C₁-C₆-alkyl, C₁-C₆-haloalkyl, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₂₋₆-alkenyl, C₂₋₆-alkynyl; R^{8b} is independently at each occurrence selected from C₁-C₄-alkyl, C₁-C₄-alkylene-R¹⁰ halo, nitro, cyano, C₁-C₄-haloalkyl, C₂-C₄-alkenyl, C₂-C₄-alkynyl, NR⁶R⁷, S(O)₂R¹⁰, S(O)R¹⁰, S(O)₂NR⁶R¹⁰, CO₂R¹⁰, C(O)R¹⁰, CONR⁶R¹⁰, OR¹⁰, SR¹⁰, 5- or 6-membered heterocycloalkyl, phenyl and 5- or 6-membered heteroaryl; R^{8d} is independently selected from H, halo, C₁-C₆-alkyl, C₁-C₆-haloalkyl, C₁-C₆-alkylene-R¹⁰, —OR¹⁰, cyano, nitro, —NR⁶R⁷, —SR¹⁰, C(O)R¹⁰, C(O)OR¹⁰, C(O)NR⁶R¹⁰, —S(O)R¹⁰, —S(O)₂R¹⁰, —S(O)₂NR⁶R¹⁰, C₃₋₆ cycloalkyl, C₂₋₆-alkenyl, C₂₋₆-alkynyl, 5-, 6-, 7-, 8-, 9- or 10-membered heterocycloalkyl, phenyl and 5- or 6-membered heteroaryl; p1 is an integer selected from 0, 1, 2, 3 and 4; and r1 is an integer selected from 0, 1 and 2.

It may be that R² has the structure:

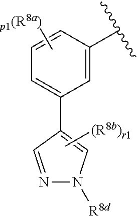

wherein R^{8a} is independently at each occurrence selected from halo, C₁-C₆-alkyl, C₁-C₆-haloalkyl, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₂₋₆-alkenyl, C₂₋₆-alkynyl; R^{8b} is independently at each occurrence selected from C₁-C₄-alkyl, C₁-C₄-alkylene-R¹⁰ halo, nitro, cyano, C₁-C₄-haloalkyl, C₂-C₄-alkenyl, C₂-C₄-alkynyl, NR⁶R⁷, S(O)₂R¹⁰, S(O)R¹⁰, S(O)₂NR⁶R¹⁰, CO₂R¹⁰, C(O)R¹⁰, CONR⁶R¹⁰, OR¹⁰, SR¹⁰, 5- or 6-membered heterocycloalkyl, phenyl and 5- or 6-membered heteroaryl; R^{8d} is independently selected from H, halo, C₁-C₆-alkyl, C₁-C₆-haloalkyl, C₁-C₆-alkylene-R¹⁰, —OR¹⁰, cyano, nitro, —NR⁶R⁷, —SR¹⁰, C(O)R¹⁰, C(O)OR¹⁰, C(O)NR⁶R¹⁰, —S(O)R¹⁰, —S(O)₂R¹⁰, —S(O)₂NR⁶R¹⁰, C₃₋₆ cycloalkyl, C₂₋₆-alkenyl, C₂₋₆-alkynyl, 5-, 6-, 7-, 8-, 9- or 10-membered heterocycloalkyl, phenyl and 5- or 6-membered heteroaryl, p1 is an integer selected from 0, 1, 2, 3 and 4; and r1 is an integer selected from 0, 1 and 2.

It may be that R² has the structure:

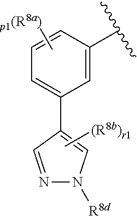

wherein R^{8a} is independently at each occurrence selected from halo, C₁-C₆-alkyl, C₁-C₆-haloalkyl, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₂₋₆-alkenyl, C₂₋₆-alkynyl; R^{8b} is independently at each occurrence selected from C₁-C₄-alkyl, C₁-C₄-alkylene-R¹⁰ halo, nitro, cyano, C₁-C₄-haloalkyl, C₂-C₄-alkenyl, C₂-C₄-alkynyl, NR⁶R⁷, S(O)₂R¹⁰, S(O)R¹⁰, S(O)₂NR⁶R¹⁰, CO₂R¹⁰, C(O)R¹⁰, CONR⁶R¹⁰, OR¹⁰, SR¹⁰; R^{8d} is independently selected from H, halo, C₁-C₆-alkyl, C₁-C₆-haloalkyl, C₁-C₆-alkylene-R¹⁰, —OR¹⁰, cyano, nitro, —NR⁶R⁷, —SR¹⁰, C(O)R¹⁰, C(O)OR¹⁰, C(O)NR⁶R¹⁰, —S(O)R¹⁰, —S(O)₂R¹⁰, —S(O)₂NR⁶R¹⁰, C₃₋₆ cycloalkyl, C₂₋₆-alkenyl, C₂₋₆-alkynyl, p1 is an integer selected from 0, 1, 2, 3 and 4; and r1 is an integer selected from 0, 1 and 2.

R² may be selected from:

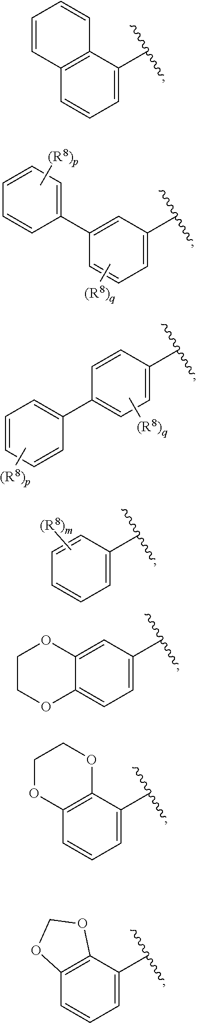

wherein m is an integer selected from 1 and 2; p is an integer independently selected from 0, 1 and 2; and q is an integer independently selected from 0 and 1.

R² may be selected from:

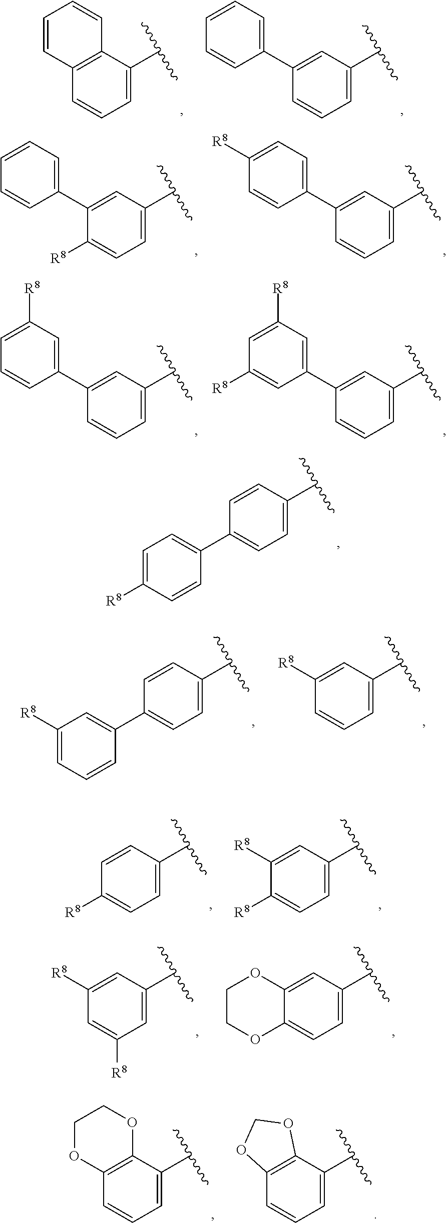

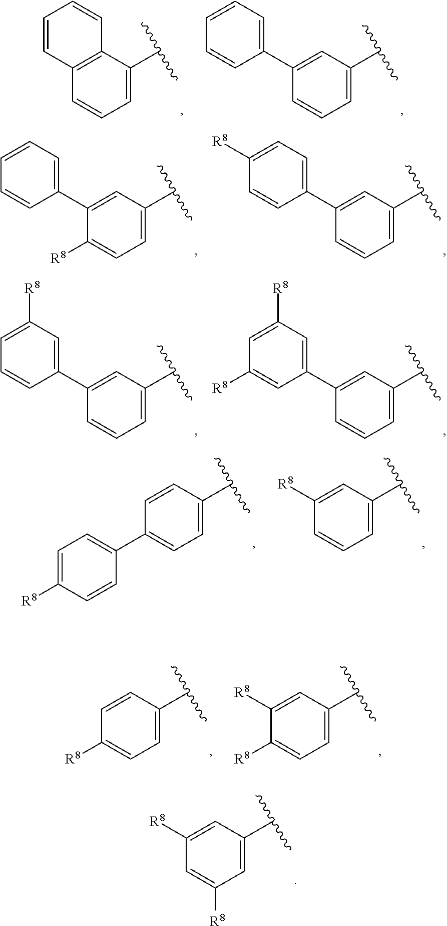

R² may be selected from:

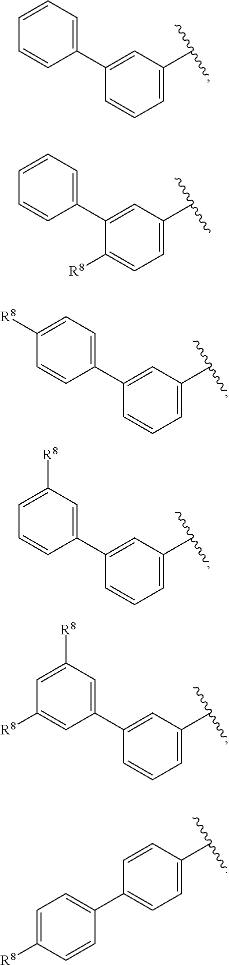

R² may be selected from:

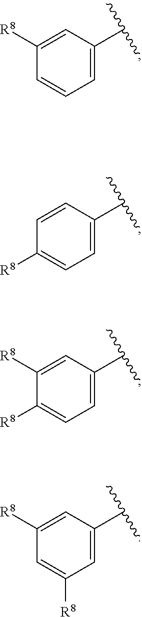

R² may be selected from:

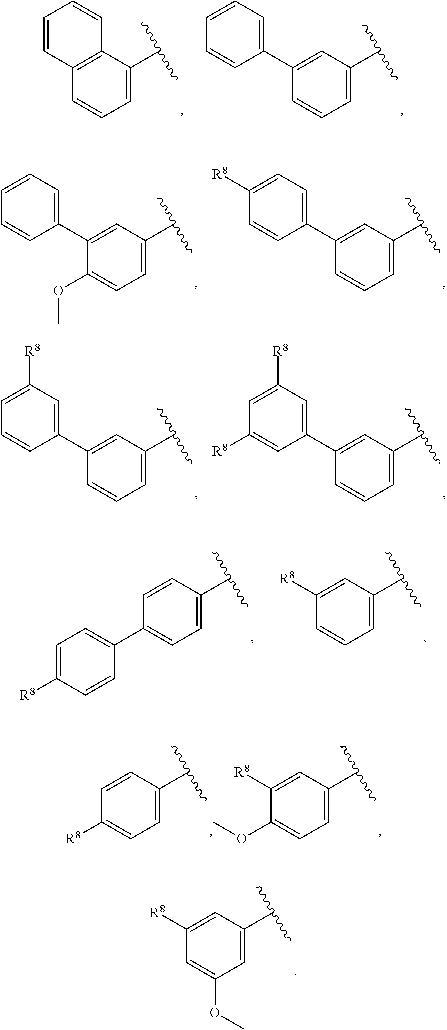

Illustrative R² groups include:

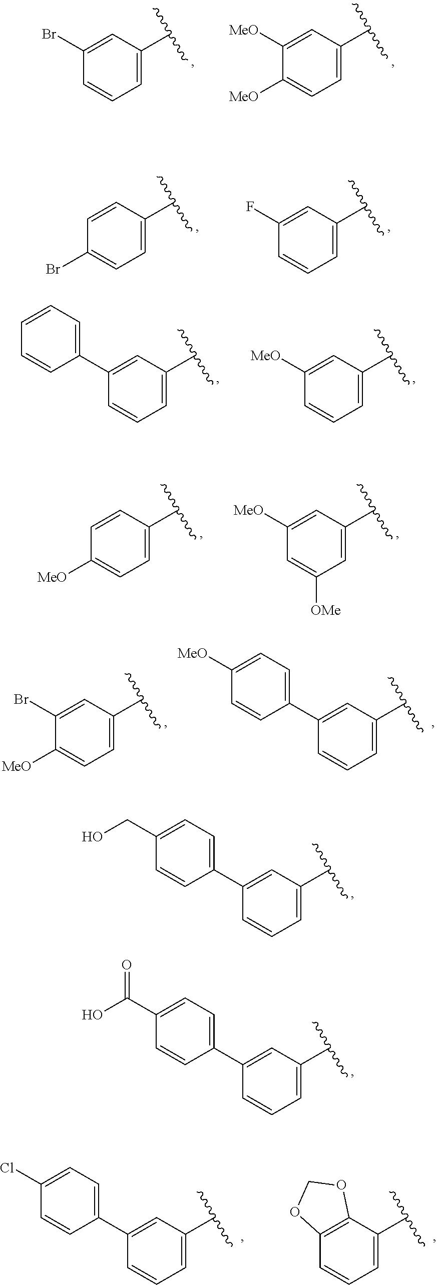

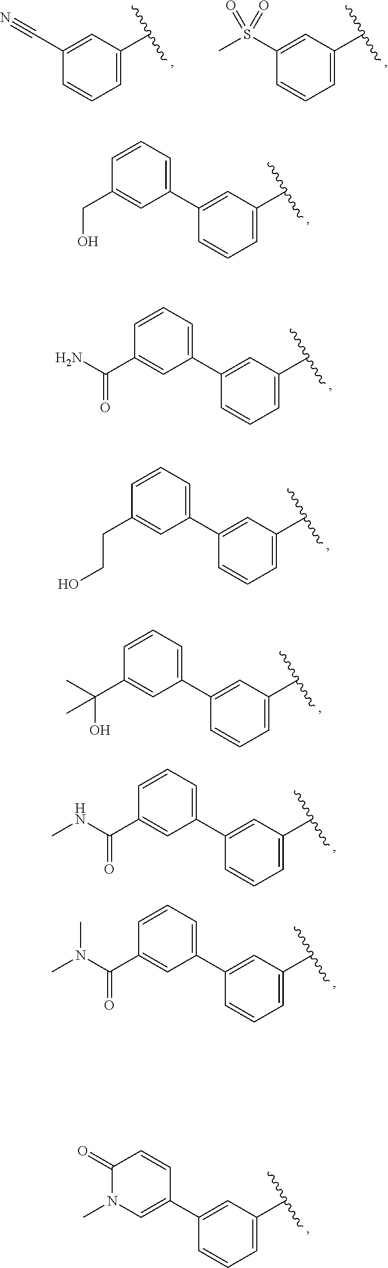

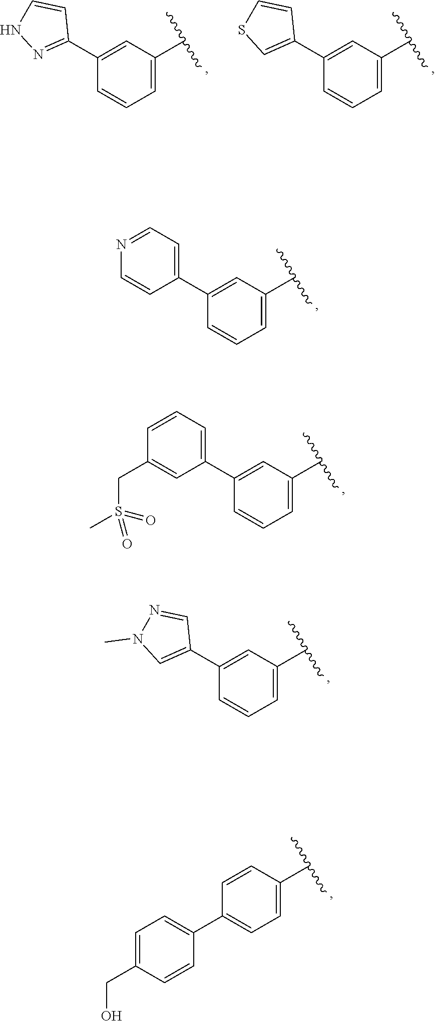 [SMILES: *c1cccc(-c2cc[nH]n2)c1 ; *c1cccc(-c2ccsc2)c1 ; *c1cccc(-c2ccncc2)c1 ; *c1cccc(-c2cccc(CS(C)(=O)=O)c2)c1 ; *c1cccc(-c2cnn(C)c2)c1 ; *c1ccc(-c2ccc(CO)cc2)cc1]

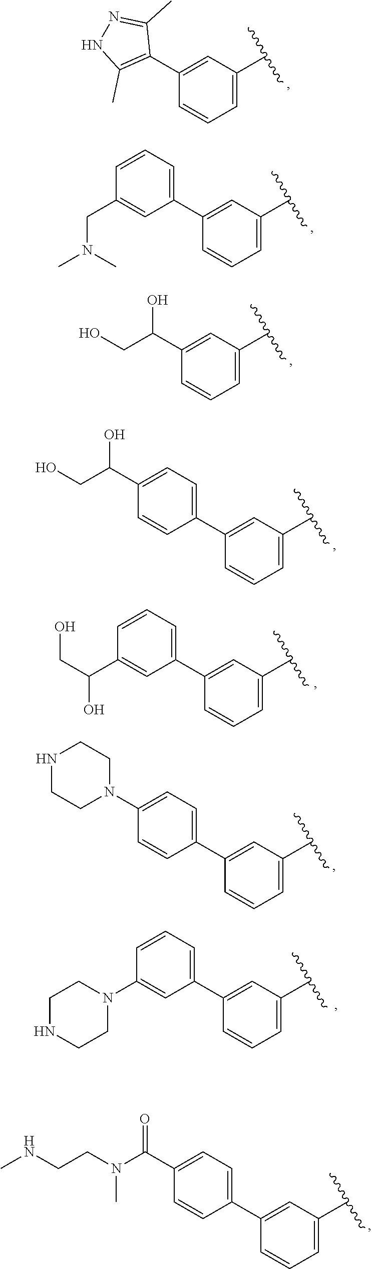

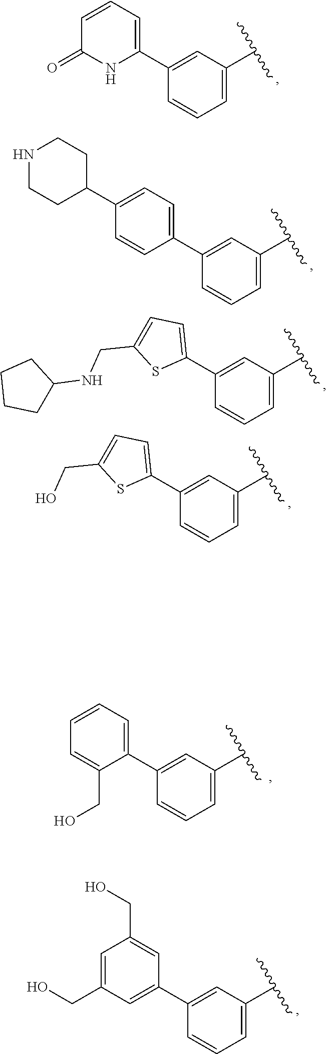

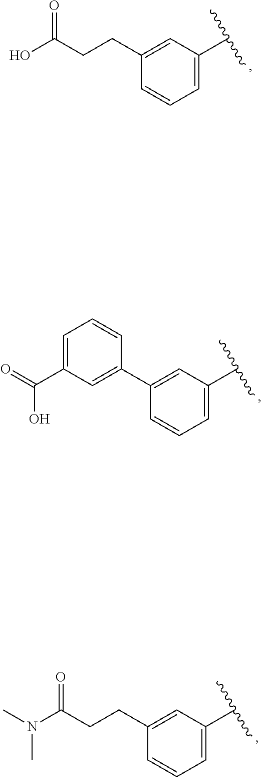 [SMILES: *c1cccc(CCC(=O)O)c1 ; *c1cccc(-c2cccc(C(=O)O)c2)c1 ; *c1cccc(CCC(=O)N(C)C)c1]

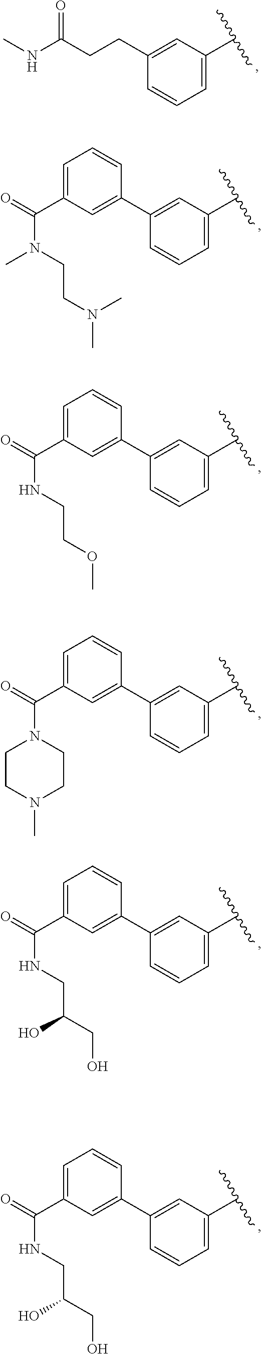

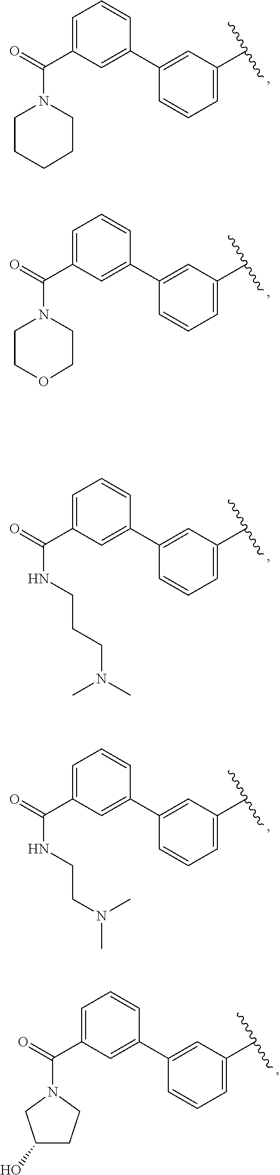

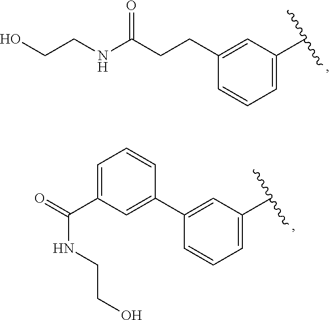 [SMILES: *c1cccc(CCC(=O)NCCO)c1 ; *c1cccc(-c2cccc(C(=O)NCCO)c2)c1]

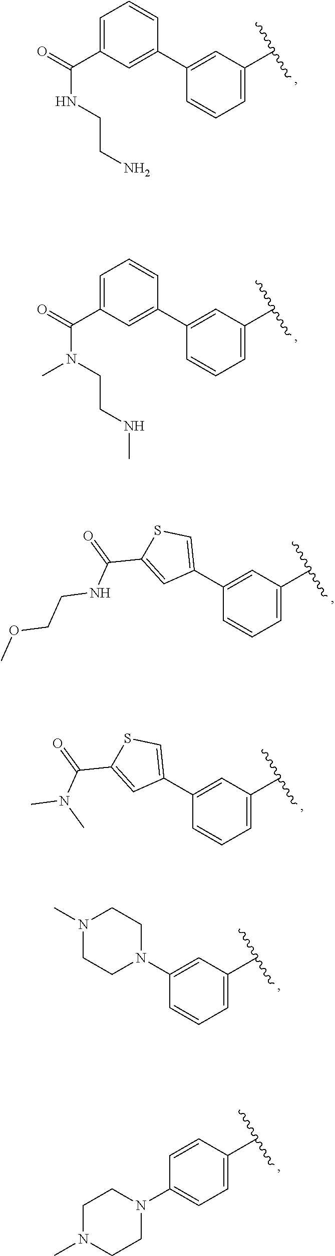 [SMILES: *c1cccc(-c2cccc(C(=O)NCCN)c2)c1 ; *c1cccc(-c2cccc(C(=O)N(C)CCNC)c2)c1 ; *c1cccc(-c2csc(C(=O)NCCOC)c2)c1 ; *c1cccc(-c2csc(C(=O)N(C)C)c2)c1 ; *c1cccc(N2CCN(C)CC2)c1 ; *c1ccc(N2CCN(C)CC2)cc1]

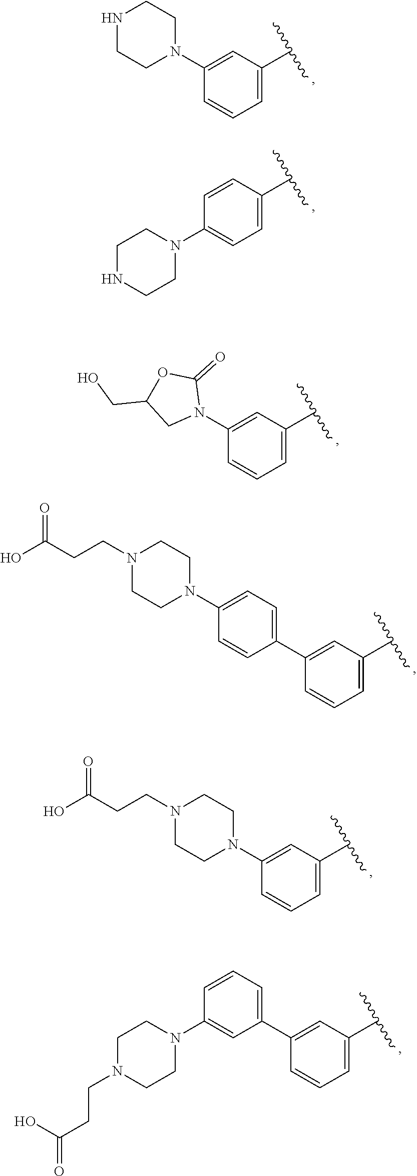 [SMILES: *c1cccc(N2CCNCC2)c1 ; *c1ccc(N2CCNCC2)cc1 ; *c1cccc(N2CC(CO)OC2=O)c1 ; *c1cccc(-c2ccc(N3CCN(CCC(=O)O)CC3)cc2)c1 ; *c1cccc(N2CCN(CCC(=O)O)CC2)c1 ; *c1cccc(-c2cccc(N3CCN(CCC(=O)O)CC3)c2)c1]

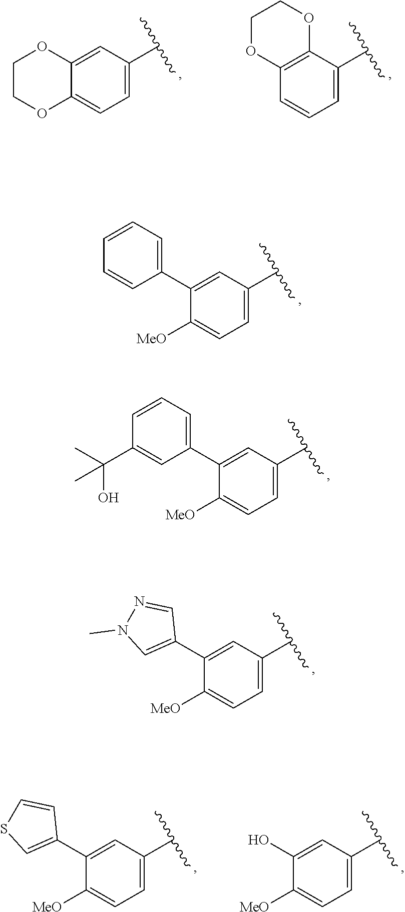

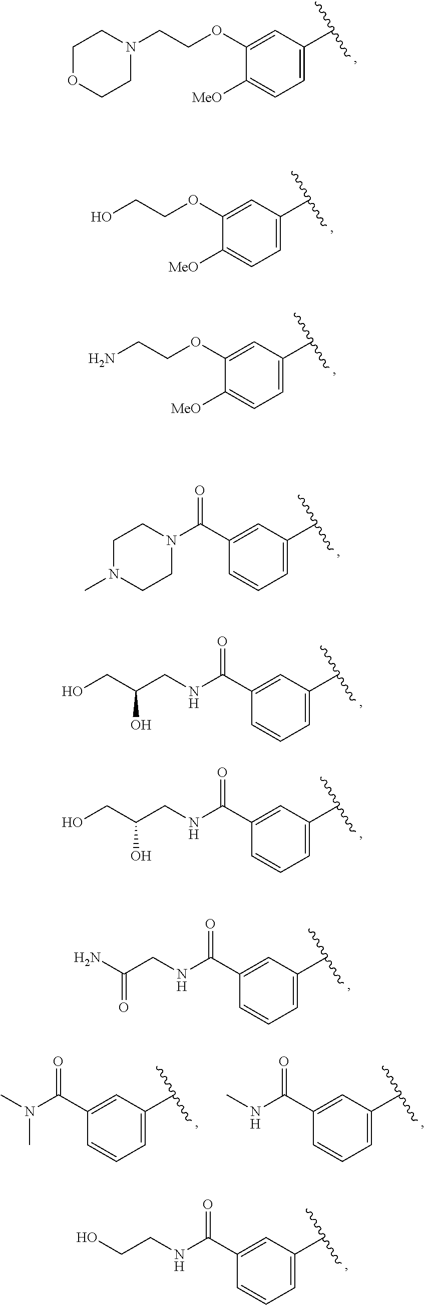

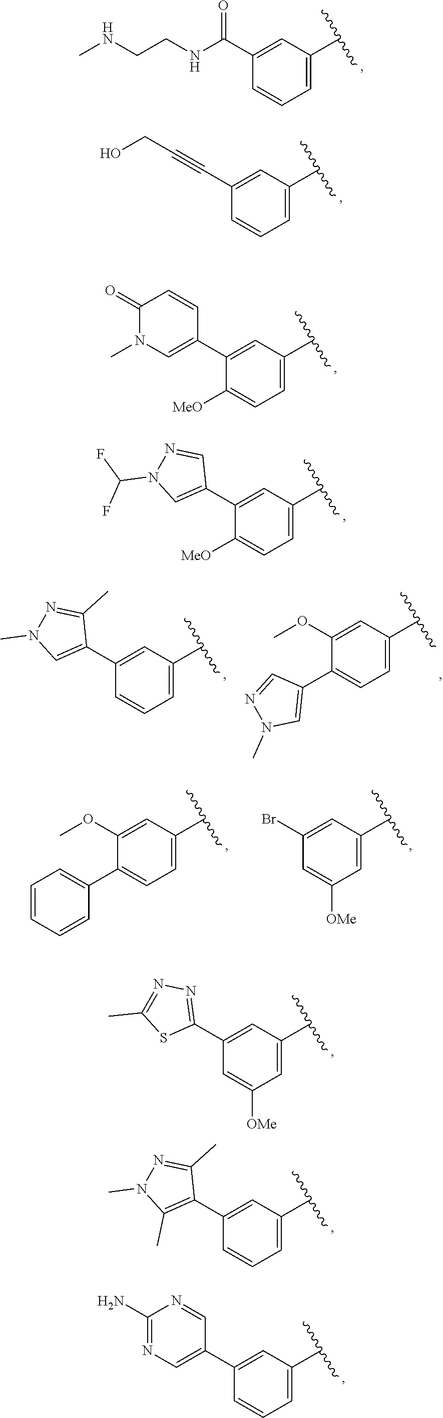

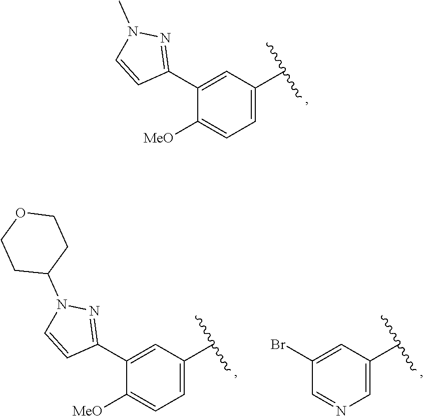 [SMILES: *c1ccc(OC)c(-c2ccn(C)n2)c1 ; *c1ccc(OC)c(-c2ccn(C3CCOCC3)n2)c1 ; *c1cncc(Br)c1]

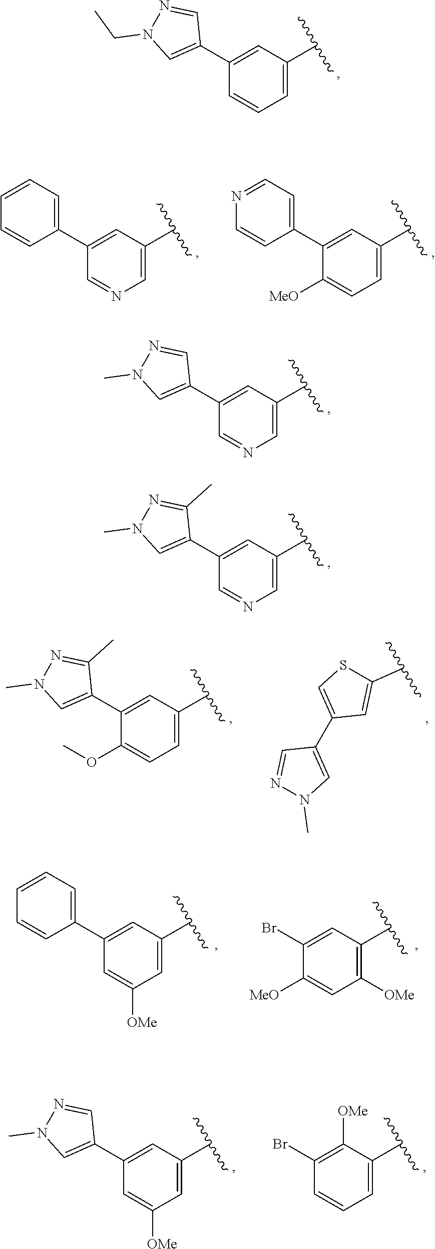

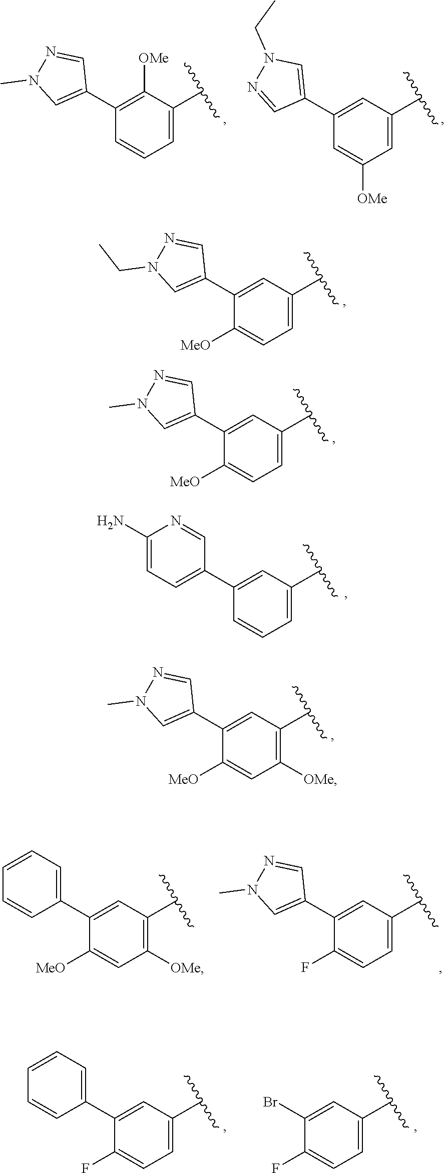

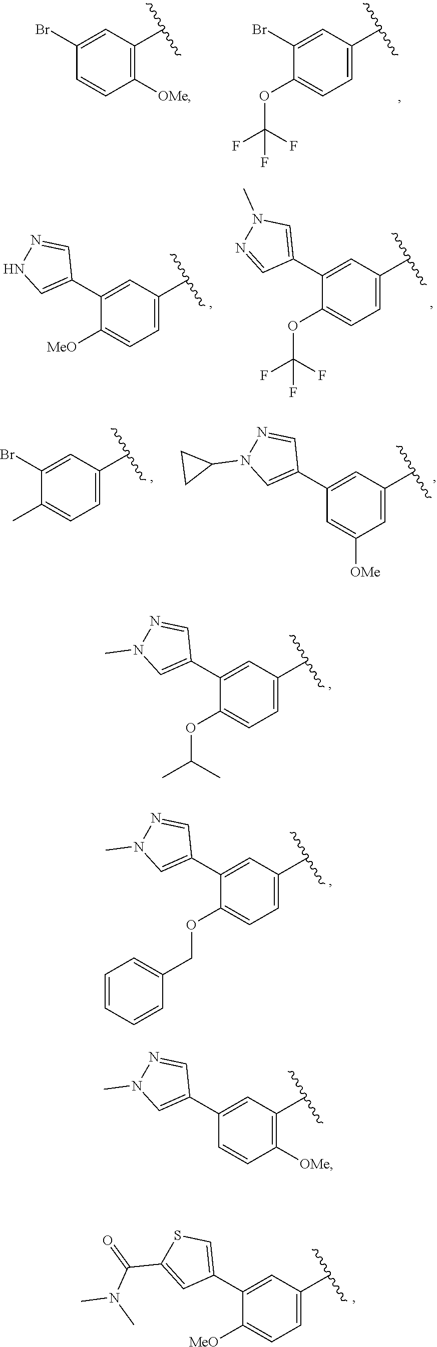

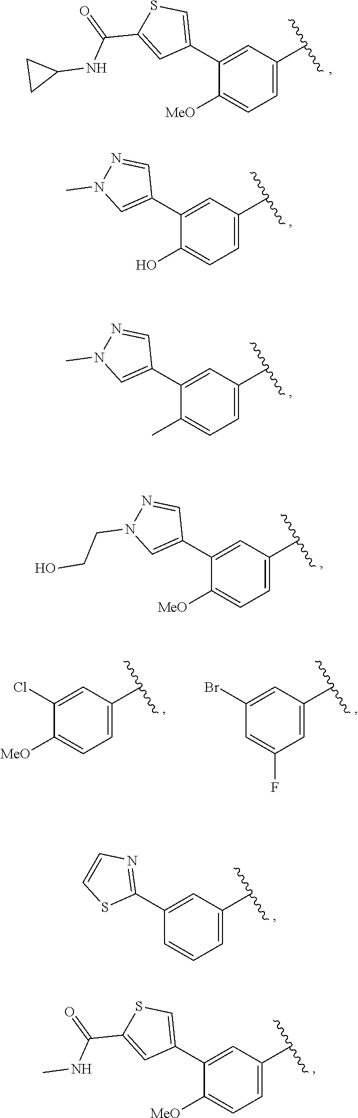

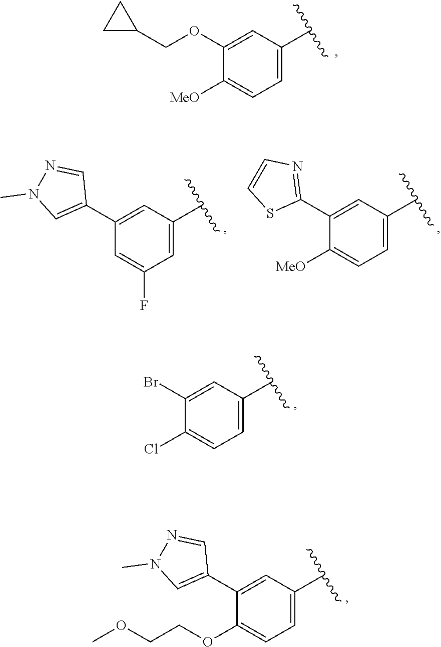 [SMILES: *c1ccc(OC)c(OCC2CC2)c1 ; *c1cc(F)cc(-c2cnn(C)c2)c1 ; *c1ccc(OC)c(-c2nccs2)c1 ; *c1ccc(Cl)c(Br)c1 ; *c1ccc(OCCOC)c(-c2cnn(C)c2)c1]

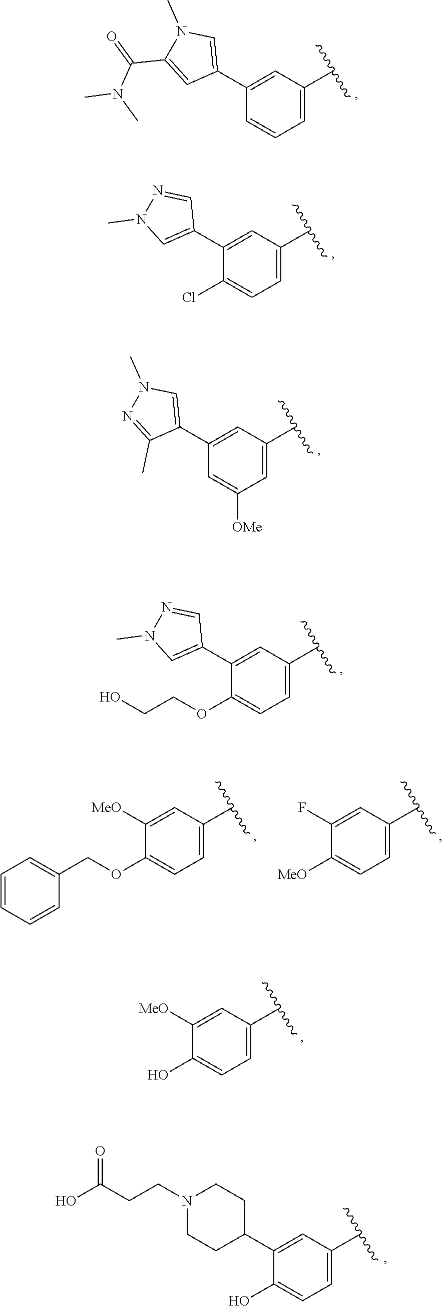

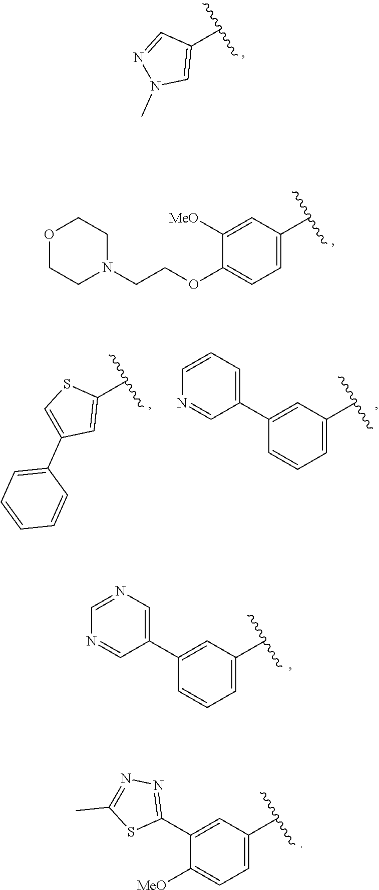

Further illustrative R² groups include:

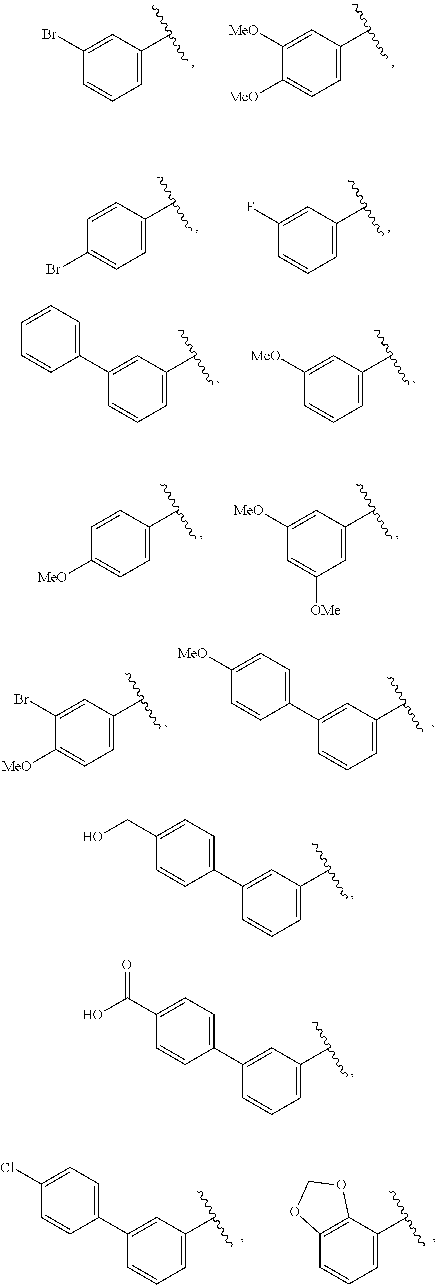

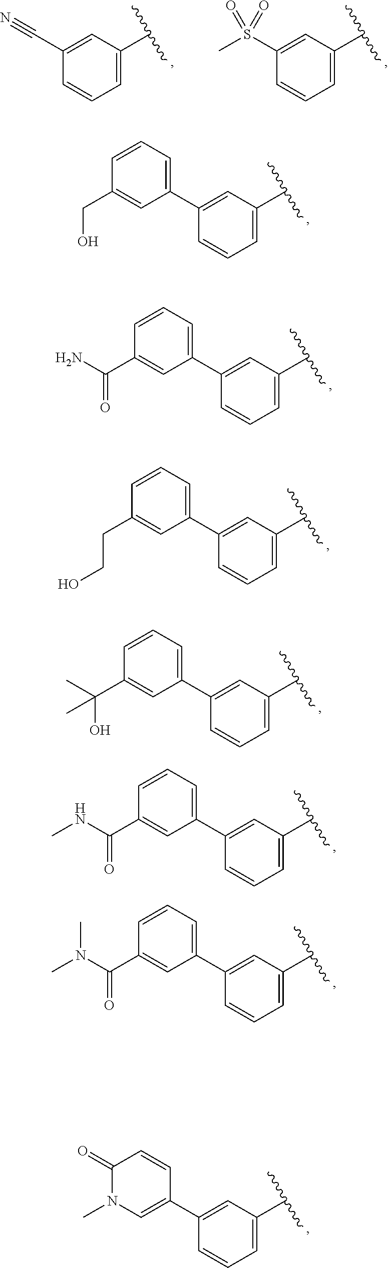

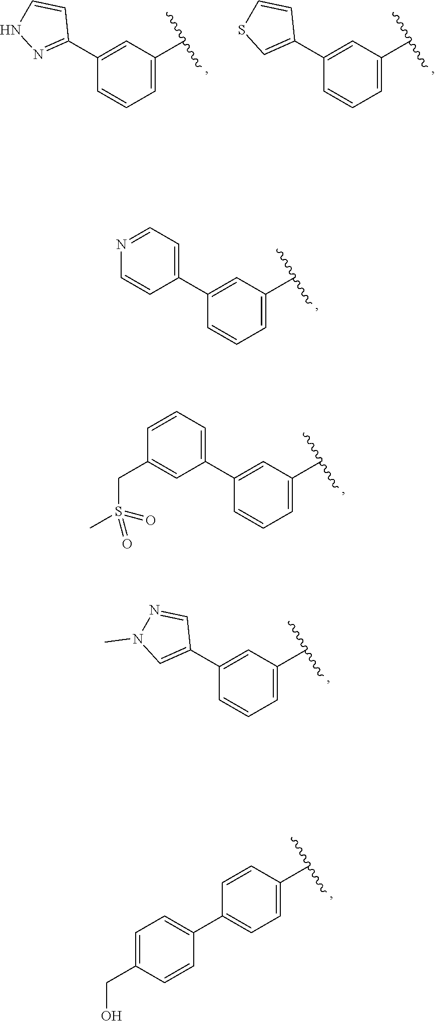 [SMILES: *c1cccc(-c2cc[nH]n2)c1 ; *c1cccc(-c2ccsc2)c1 ; *c1cccc(-c2ccncc2)c1 ; *c1cccc(-c2cccc(CS(C)(=O)=O)c2)c1 ; *c1cccc(-c2cnn(C)c2)c1 ; *c1ccc(-c2ccc(CO)cc2)cc1]

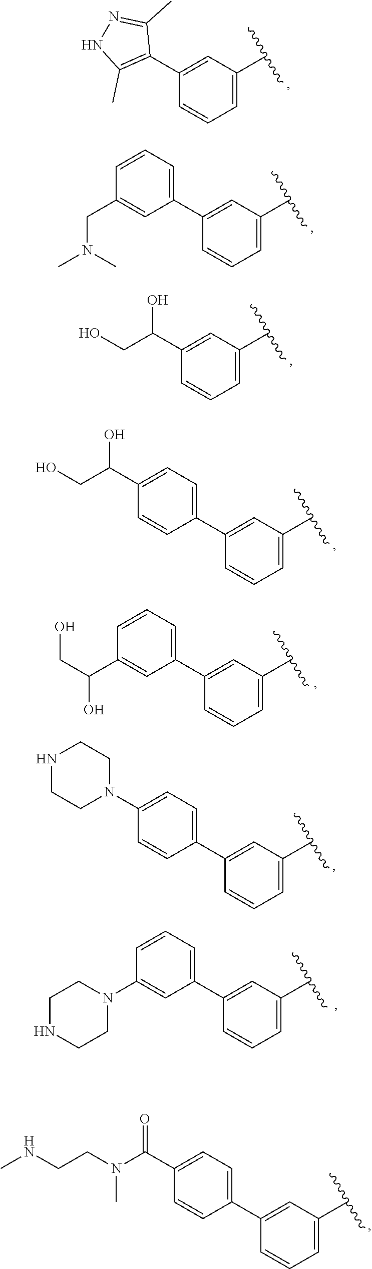

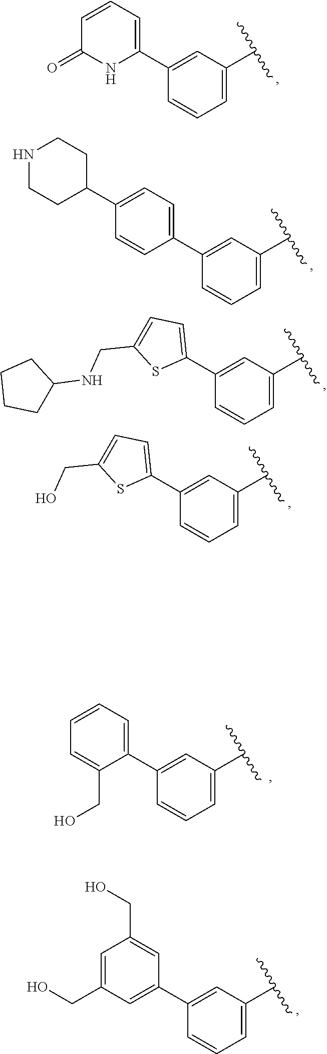

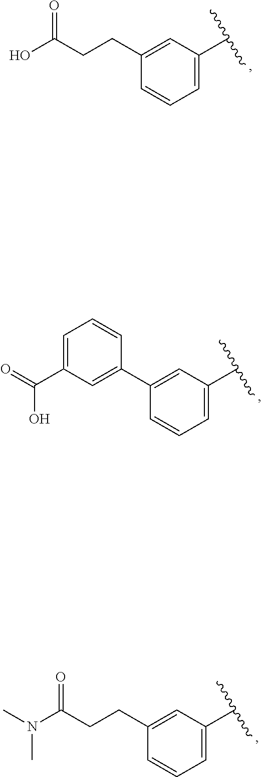 [SMILES: *c1cccc(CCC(=O)O)c1 ; *c1cccc(-c2cccc(C(=O)O)c2)c1 ; *c1cccc(CCC(=O)N(C)C)c1]

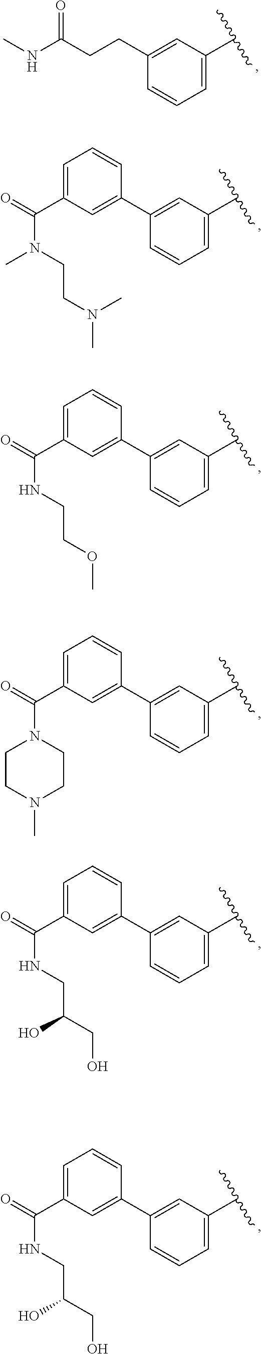

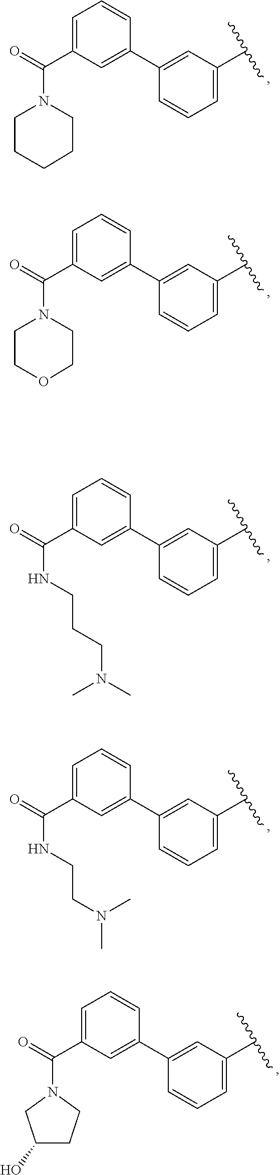

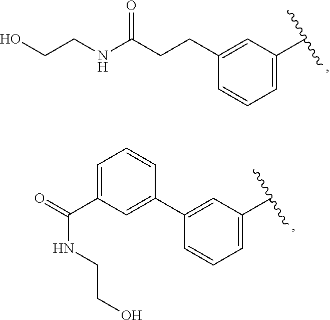 [SMILES: *c1cccc(CCC(=O)NCCO)c1 ; *c1cccc(-c2cccc(C(=O)NCCO)c2)c1]

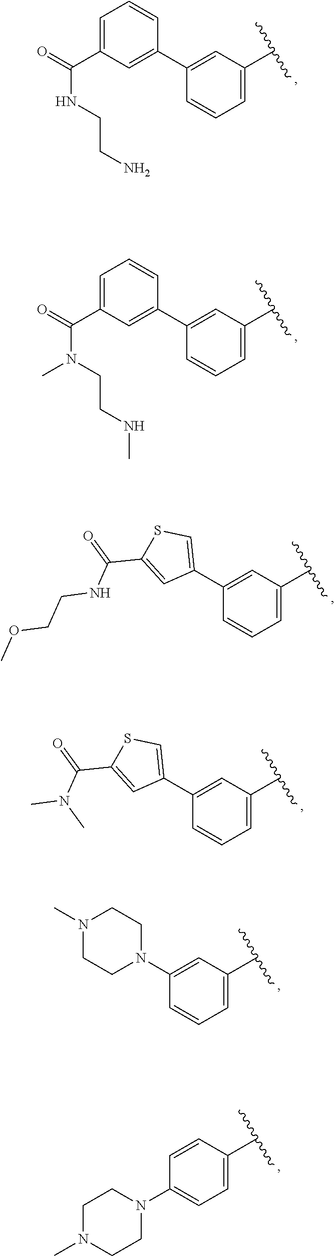 [SMILES: *c1cccc(-c2cccc(C(=O)NCCN)c2)c1 ; *c1cccc(-c2cccc(C(=O)N(C)CCNC)c2)c1 ; *c1cccc(-c2csc(C(=O)NCCOC)c2)c1 ; *c1cccc(-c2csc(C(=O)N(C)C)c2)c1 ; *c1cccc(N2CCN(C)CC2)c1 ; *c1ccc(N2CCN(C)CC2)cc1]

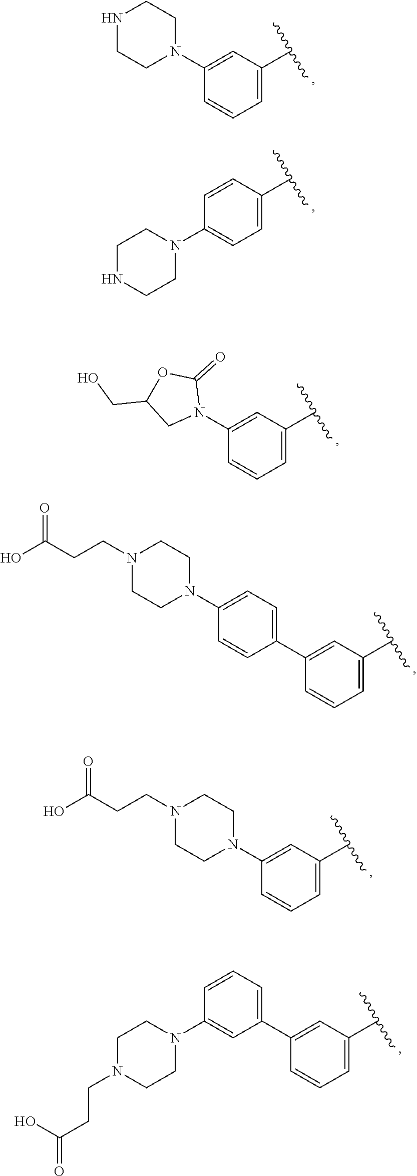 [SMILES: *c1cccc(N2CCNCC2)c1 ; *c1ccc(N2CCNCC2)cc1 ; *c1cccc(N2CC(CO)OC2=O)c1 ; *c1cccc(-c2ccc(N3CCN(CCC(=O)O)CC3)cc2)c1 ; *c1cccc(N2CCN(CCC(=O)O)CC2)c1 ; *c1cccc(-c2cccc(N3CCN(CCC(=O)O)CC3)c2)c1]

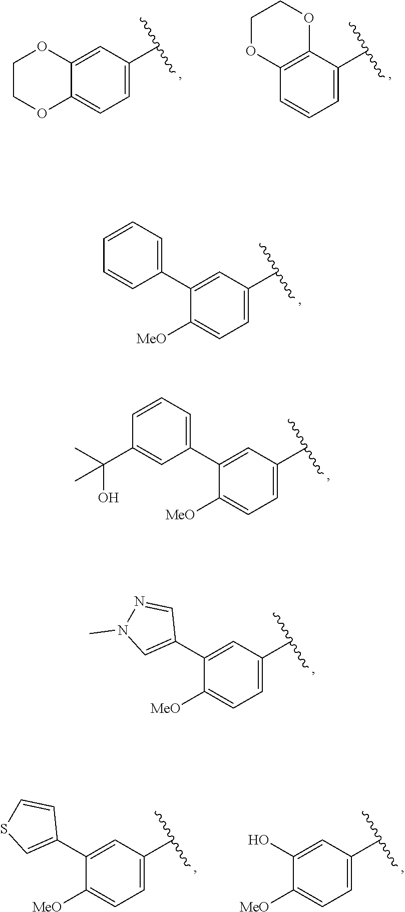

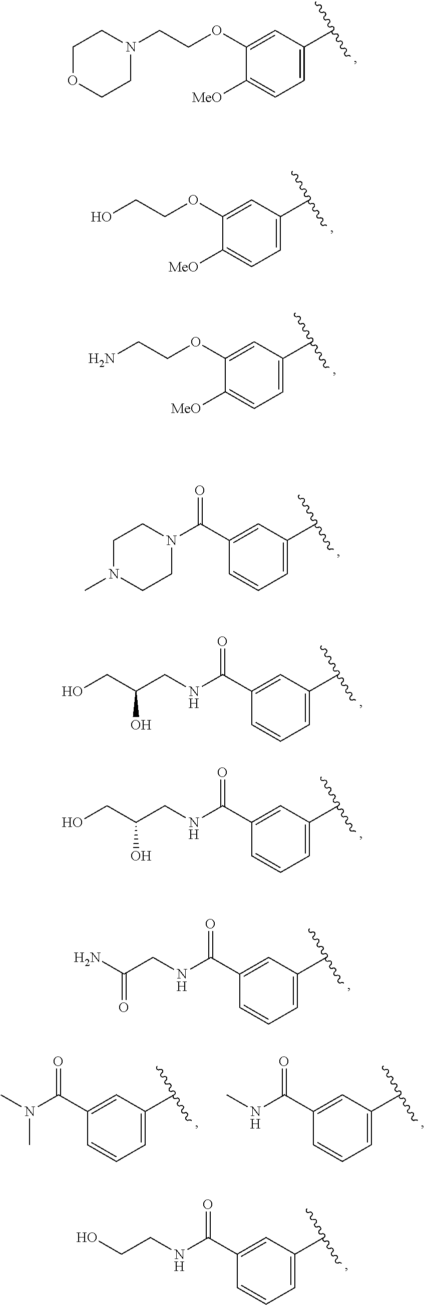

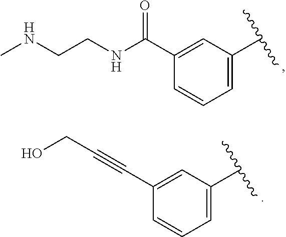 [SMILES: *c1cccc(C(=O)NCCNC)c1 ; *c1cccc(C#CCO)c1]

Further illustrative R² groups include:

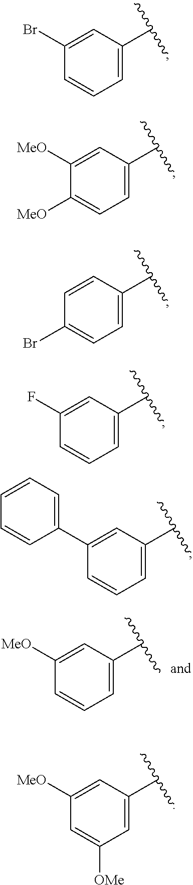

It may be that R³ is H. It may be that R³ is —C₁₋₆ alkyl, e.g. methyl, ethyl, propyl. It may be that R³ is H and Y is —C(O)—. It may be that L¹ is absent, R³ is H and Y is —C(O)—

R⁴ may be independently selected at each occurrence from the group comprising: halo, C₁-C₆-alkyl, C₁-C₆-haloalkyl, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₂₋₆-alkenyl, C₂₋₆-alkynyl. R⁴ may be independently selected at each occurrence from the group comprising: halo, C₁-C₄-alkyl, C₁-C₄-haloalkyl, —OR¹⁰, cyano, nitro and —NR⁶R⁷.

R^{4a} may be independently selected at each occurrence from the group comprising: halo, C₁-C₆-alkyl, C₁-C₆-haloalkyl, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₂₋₆-alkenyl and C₂₋₆-alkynyl. R^{4a} may be independently selected at each occurrence from the group comprising: halo, C₁-C₄-alkyl, C₁-C₄-haloalkyl, —OR¹⁰, cyano, nitro and —NR⁶R⁷. R^{4a} may be independently selected at each occurrence from the group comprising: C₁-C₄-alkyl and C₁-C₄-haloalkyl. R^{4a} may be independently C₁-C₄-alkyl. R^{4a} may be methyl.

R⁵ may be selected from the group comprising: —C(O)NR⁶R¹⁴, —C(O)R¹², phenyl, 6-membered heteroaryl; 5-, 6-, or 7- or 8-membered heterocycloalkyl and cyclopropyl; wherein any said phenyl or heteroaryl group may be optionally substituted with at least one R⁸ group; or wherein any said heterocycloalkyl or cyclopropyl may be optionally substituted with at least one R⁹ group.

R⁵ may be selected from the group comprising: —C(O)NR⁶R¹⁴ and —C(O)R¹². R⁵ may be —C(O)NR⁶R¹⁴, e.g. —C(O)NHR¹⁴ or —C(O)MeR¹⁴. R⁵ may be —C(O)R¹², e.g. —C(O)— piperidyl or —C(O)-piperazinyl.

R⁵ may be selected from the group comprising phenyl, 6-membered heteroaryl, 5-, 6- or 7- or 8-membered heterocycloalkyl and cyclopropyl; wherein any said phenyl or heteroaryl group is optionally substituted with at least one R⁸ group; or wherein any said heterocycloalkyl or cyclopropyl is optionally substituted with at least one R⁹ group.

R⁵ may be selected from the group comprising —C(O)NR⁶R¹⁴, —C(O)R¹², 5-, 6-, 7-, 8-, 9- or 10-membered heterocycloalkyl and cyclopropyl; wherein any said heterocycloalkyl or cyclopropyl is optionally substituted with at least one R⁹ group. In embodiments, the heterocycloalkyl or cyclopropyl group is a saturated ring system.

R⁵ may be selected from the group comprising phenyl, 6-membered heteroaryl, 5-, 6-, 7-, 8-, 9- or 10-membered heterocycloalkyl and cyclopropyl; wherein any said phenyl or heteroaryl group is optionally substituted with at least one R⁸ group; or wherein any said heterocycloalkyl or cyclopropyl is optionally substituted with at least one R⁹ group.

R⁵ may be selected from the group comprising 5-, 6-, 7-, 8-, 9- or 10-membered heterocycloalkyl and cyclopropyl; wherein any said heterocycloalkyl or cyclopropyl is optionally substituted with at least one R⁹ group. In embodiments, the heterocycloalkyl or cyclopropyl group is a saturated ring system.

R⁵ may be selected from the group comprising phenyl and 6-membered heteroaryl; wherein any said phenyl or heteroaryl group is optionally substituted with at least one R⁸ group.

R⁵ may be cyclopropyl; wherein said cyclopropyl is optionally substituted with at least one R⁹ group.

R⁵ may be a 5-, 6-, 7-, 8-, 9- or 10-membered heterocycloalkyl; wherein said heterocycloalkyl is optionally substituted with at least one R⁹ group. R⁵ may be 5-, 6- or 7- or 8-membered heterocycloalkyl; wherein said heterocycloalkyl is optionally substituted with at least one R⁹ group. R⁵ may be a 6- or 7-membered heterocycloalkyl, wherein said heterocycloalkyl is optionally substituted with at least one R⁹ group. Preferably, R⁵ is a 6-membered heterocycloalkyl, wherein said heterocycloalkyl is optionally substituted with at least one R⁹ group.

R⁵ may be a 7-, 8-, 9- or 10-membered bicyclic heterocycloalkyl; wherein said heterocycloalkyl is optionally substituted with at least one R⁹ group. R⁵ may be a 8-, 9- or 10-membered bicyclic heterocycloalkyl; wherein said heterocycloalkyl is optionally substituted with at least one R⁹ group. The bicyclic heterocycloalkyl may be a fused bicycle. The bicyclic heterocycloalkyl may be a spiro-fused bicycle. The bicyclic heterocycloalkyl may be a bridged bicycle.

When R⁵ is a heterocycloalkyl group, said heterocycloalkyl group may include at least two heteroatoms. It may be that said heterocycloalkyl group includes one N atom and at least one other heteroatom independently selected from O, N and S. It may be that said heterocycloalkyl group contains two N atoms. It may be that the heterocycloalkyl group does not contain S or O.

When R⁵ is a heterocycloalkyl group having at least one N atom in the ring, it may be that said heterocycloalkyl group is attached to the X⁴ group via the N atom.

R⁵ may be phenyl, pyridyl, pyrazyl, pyrazolyl, pyridazyl, pyrimidyl, pyrrolidyl, imidazolinyl, pyrazolidyl, tetrahydrothiophenyl, dihydropyrrolyl, piperidyl, piperazinyl, morpholinyl, thiomorpholinyl, tetrahydropyran, tetrahydropyridyl, azepanyl, diazepanyl, azaspiroheptanyl, diazaspiroheptanyl, azabicycloheptyl, diazabicycloheptyl, diazabicyclooctyl, octahydropyrrolopyrazyl, cyclopropyl, —C(O)R¹² or —C(O)NR⁶R¹⁴. R⁵ may be phenyl, pyridyl, pyrazyl, pyridazyl, pyrimidyl, pyrrolidyl, imidazolinyl, pyrazolidyl, tetrahydrothiophenyl, piperidyl, piperazinyl, morpholinyl, thiomorpholinyl, tetrahydropyran, azepanyl, diazepanyl, azaspiroheptanyl, diazaspiroheptanyl, cyclopropyl, —C(O)R¹² or —C(O)NR⁶R¹⁴. R⁵ may be phenyl, pyridyl, pyrrolidyl, piperidyl, piperazinyl, thiomorpholinyl, morpholinyl, diazepanyl, diazespiroheptanyl, cyclopropyl, —C(O)R¹² or —C(O)NR⁶R¹⁴. R⁵ may be pyridyl, piperidyl, piperazinyl, thiomorpholinyl, morpholinyl, diazepanyl, —C(O)R¹² or —C(O)NR⁶R¹⁴. R⁵ may be piperazinyl.

R⁵ may be phenyl, pyridyl, pyrazyl, pyrazolyl, pyridazyl, pyrimidyl, pyrrolidyl, imidazolinyl, pyrazolidyl, tetrahydrothiophenyl, dihydropyrrolyl, piperidyl, piperazinyl, morpholinyl, thiomorpholinyl, tetrahydropyran, tetrahydropyridyl, azepanyl, diazepanyl, azaspiroheptanyl, diazaspiroheptanyl, azabicycloheptyl, diazabicycloheptyl, diazabicyclooctyl, octahydropyrrolopyrazyl, or cyclopropyl. R⁵ may be phenyl, pyridyl, pyrazyl, pyridazyl, pyrimidyl, pyrrolidyl, imidazolinyl, pyrazolidyl, tetrahydrothiophenyl, piperidyl, piperazinyl, morpholinyl, thiomorpholinyl, tetrahydropyran, azepanyl, diazepanyl, azaspiroheptanyl, diazaspiroheptanyl, or cyclopropyl. R⁵ may be phenyl, pyridyl, pyrrolidyl, piperidyl, piperazinyl, thiomorpholinyl, morpholinyl, diazepanyl, diazespiroheptanyl or cyclopropyl. R⁵ may be pyridyl, piperidyl, piperazinyl, thiomorpholinyl, morpholinyl, or diazepanyl. R⁵ may be piperazinyl.

R⁵ may be phenyl, pyridyl, pyrazyl, pyridazyl, pyrimidyl. R⁵ may be phenyl or pyridyl.

R⁵ may be pyrrolidyl, imidazolinyl, pyrazolidyl, tetrahydrothiophenyl, dihydropyrrolyl, piperidyl, piperazinyl, morpholinyl, thiomorpholinyl, tetrahydropyran, tetrahydropyridyl, azepanyl, diazepanyl, azaspiroheptanyl, diazaspiroheptanyl, azabicycloheptyl, diazabicycloheptyl, diazabicyclooctyl, or octahydropyrrolopyrazyl. R⁵ may be pyrrolidyl, imidazolinyl, pyrazolidyl, tetrahydrothiophenyl, piperidyl, piperazinyl, morpholinyl, thiomorpholinyl, tetrahydropyran, azepanyl, diazepanyl, azaspiroheptanyl, or diazaspiroheptanyl. R⁵ may be pyrrolidyl, piperidyl, piperazinyl, thiomorpholinyl, morpholinyl, diazepanyl, or diazespiroheptanyl. R⁵ may be piperidyl, piperazinyl, thiomorpholinyl, morpholinyl, or diazepanyl. R⁵ may be piperazinyl.

R⁵ may be imidazolinyl, pyrazolidyl, piperazinyl, morpholinyl, thiomorpholinyl, diazepanyl, or diazaspiroheptanyl. R⁵ may be piperazinyl, thiomorpholinyl, morpholinyl, diazepanyl, or diazespiroheptanyl. R⁵ may be piperazinyl.

R⁵ may be selected from: phenyl, pyridyl, piperidine substituted with R^{9b}, piperazine substituted with R^{9b}, diazabicycloheptyl substituted with R^{9b}, diazabicyclooctyl substituted with R^{9b}, tetrahydropyridyl, morpholine, thiomorpholine, 1,4-diazepanyl, pyrrolidinyl, cyclopropyl and diazaspiroheptanyl; wherein, when R⁵ is phenyl or pyridyl, R⁵ may be substituted where chemically possible with 0, 1, 2, 3, 4, or 5 R⁸ groups; and wherein, when R⁵ is piperidine substituted with R^{9b}, piperazine substituted with R^{9b}, diazabicycloheptyl substituted with R^{9b}, diazabicyclooctyl substituted with R^{9b}, tetrahydropyridyl, morpholine, thiomorpholine, 1,4-diazepanyl, pyrrolidinyl, cyclopropyl or diazaspiroheptanyl, R⁵ may be substituted where chemically possible with 0, 1, 2, 3, 4, 5, or 6 R⁹ groups.

R⁵ may be selected from: phenyl, pyridyl, piperidine substituted with R^{9b}, piperazine substituted with R^{9b}, morpholine, thiomorpholine, 1,4-diazepanyl, pyrrolidinyl, cyclopropyl and diazaspiroheptanyl; wherein, when R⁵ is phenyl or pyridyl, R⁵ may be substituted where chemically possible with 0, 1, 2, 3, 4, or 5 R⁸ groups; and wherein, when R⁵ is piperidine substituted with R^{9b}, piperazine substituted with R^{9b}, morpholine, thiomorpholine, 1,4-diazepanyl, pyrrolidinyl, cyclopropyl or diazaspiroheptanyl, R⁵ may be substituted where chemically possible with 0, 1, 2, 3, 4, 5, or 6 R⁹ groups.

R⁵ may be selected from:

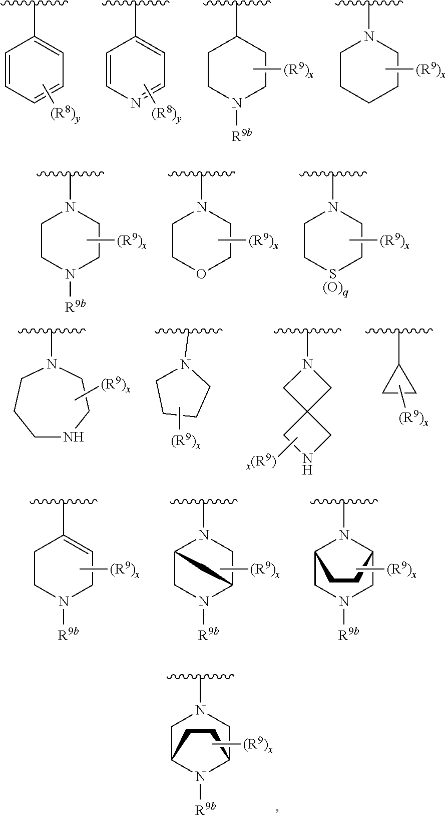

wherein x is selected from 0, 1, 2, 3, 4, 5 or 6; y is selected from 0, 1, 2, 3, 4, or 5; and q is selected from 0, 1 or 2.

R⁵ may be selected from:

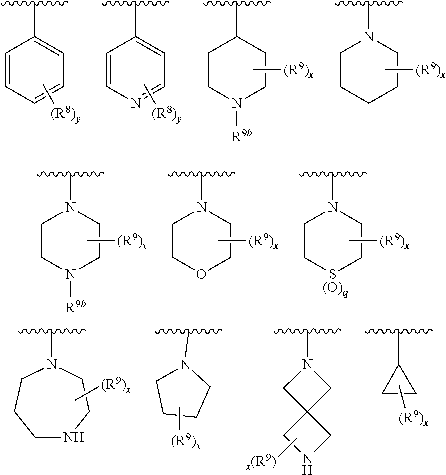

wherein x is selected from 0, 1, 2, 3, 4, 5 or 6; y is selected from 0, 1, 2, 3, 4, or 5; and q is selected from 0, 1 or 2.

R⁵ may be selected from:

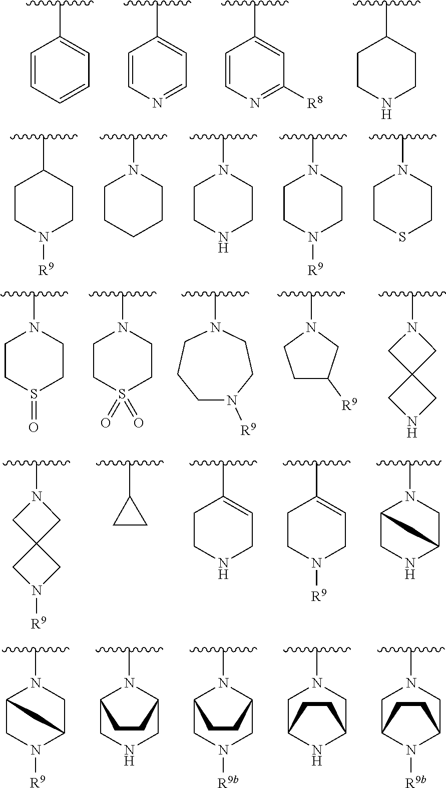

R⁵ may be selected from:

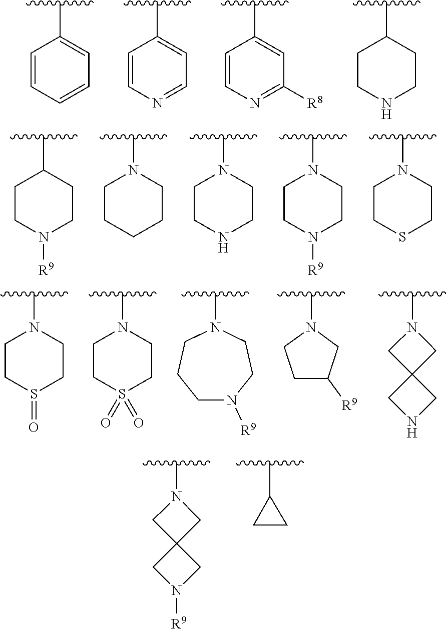

R⁵ may be selected from: piperidine substituted with R^{9b}, piperazine substituted with R^{9b}, morpholine, thiomorpholine, 1,4-diazepanyl, pyrrolidinyl, and diazaspiroheptanyl; wherein R⁵ may be substituted where chemically possible with 0, 1, 2, 3, 4, 5, or 6R⁹ groups.

R⁵ may be selected from:

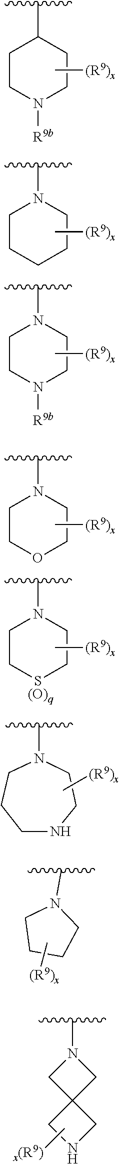

wherein x is selected from 0, 1, 2, 3, 4, 5 or 6 and q is selected from 0, 1 or 2. R⁵ may be selected from: piperidine substituted with R^{9b}, piperazine substituted with R^{9b}, morpholine or thiomorpholine; wherein R⁵ may be substituted where chemically possible with 0, 1, 2, 3, 4, 5, or 6R⁹ groups.

R⁵ may be selected from: piperazine substituted with R^{9b}, morpholine or thiomorpholine; wherein R⁵ may be substituted where chemically possible with 0, 1, 2, 3, 4, 5, or 6 R⁹ groups.

R⁵ may be selected from:

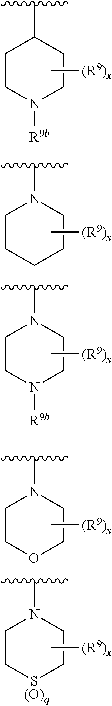

wherein x is selected from 0, 1, 2, 3, 4, 5 or 6 and q is selected from 0, 1 or 2.

R⁵ may be selected from:

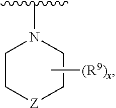

wherein Z is NR^{9b}, O or S(O)_{q}; wherein x is selected from 0, 1, 2, 3, 4, 5 or 6 and q is selected from 0, 1 or 2. It may be that Z is NR^{b}. It may be that Z is O. It may be that Z is S(O)_{q}.

R⁵ may be:

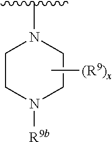

wherein x is selected from 0, 1, 2, 3, 4, 5 or 6, and R^{9b} is selected from the group comprising: C₁-C₆-alkyl, C₁-C₆-haloalkyl, C(O)R⁶, C(O)OR⁶, C(O)NR⁶R⁶, —S(O)R⁶, —S(O)₂R⁶, —S(O)₂NR⁶R⁶, C₃₋₆ cycloalkyl, 4-, 5- or 6-membered heterocycloalkyl, C₂₋₆-alkenyl C₂₋₆-alkynyl, C₂-C₃-alkylene-R^{9a} and CH₂-cyclopropyl. x may be 1. x may be 0. R^{9b} may be selected from the group comprising: H, C₁₋₄ alkyl, C(O)R⁶, C₂-C₃-alkylene-R^{9a} and CH₂-cyclopropyl. Preferably, R^{9b} is C₁₋₄ alkyl.

R⁵ may be:

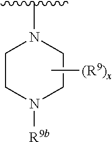

wherein x is selected from 0, 1, 2, 3, 4, 5 or 6, and R^{9b} is selected from the group comprising: C₁-C₆-alkyl, C₁-C₆-haloalkyl, C(O)R¹⁰, C(O)OR¹⁰, C(O)NR⁶R¹⁰, —S(O)R¹⁰, —S(O)₂R¹⁰, —S(O)₂NR⁶R¹⁰, C₃₋₆ cycloalkyl, 4-, 5- or 6-membered heterocycloalkyl, C₂₋₆-alkenyl C₂₋₆-alkynyl, C₂-C₃-alkylene-R^{9a} and CH₂-cyclopropyl. x may be 1. x may be 0. R^{9b} may be selected from the group comprising: H, C₁₋₄ alkyl, C(O)R¹⁰, C₂-C₃-alkylene-R^{9a} and CH₂-cyclopropyl. Preferably, R^{9b} is C₁₋₄ alkyl.

R⁵ may be:

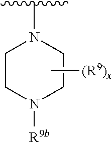

wherein x is selected from 0, 1, 2, 3, 4, 5 or 6, and R^{9b} is selected from the group comprising: C₁-C₆-alkyl, C₁-C₆-haloalkyl, C(O)R⁶, C(O)OR⁶, C(O)NR⁶R⁶, —S(O)R⁶, —S(O)₂R⁶, —S(O)₂NR⁶R⁶, C₃₋₆ cycloalkyl, 4-, 5- or 6-membered heterocycloalkyl, C₂₋₆-alkenyl C₂₋₆-alkynyl and C₂-C₃-alkylene-R^{9a}. x may be 1. x may be 0. R^{9b} may be selected from the group comprising: H, C₁₋₄ alkyl, C(O)R⁶, and C₂-C₃-alkylene-R^{9a} Preferably, R^{9b} is C₁₋₄ alkyl.

R⁵ may be:

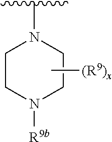

wherein x is selected from 0, 1, 2, 3, 4, 5 or 6, and R^{9b} is selected from the group comprising: C₁-C₆-alkyl, C₁-C₆-haloalkyl, C(O)R¹⁰, C(O)OR¹⁰, C(O)NR⁶R¹⁰, —S(O)R¹⁰, —S(O)₂R¹⁰, —S(O)₂NR⁶R¹⁰, C₃₋₆ cycloalkyl, 4-, 5- or 6-membered heterocycloalkyl, C₂₋₆-alkenyl C₂₋₆-alkynyl and C₂-C₃-alkylene-R^{9a}. x may be 1. x may be 0. R^{9b} may be selected from the group comprising: H, C₁₋₄ alkyl, C(O)R¹⁰, and C₂-C₃-alkylene-R^{9a} Preferably, R^{9b} is C₁₋₄ alkyl.

Illustrative R⁵ groups include:

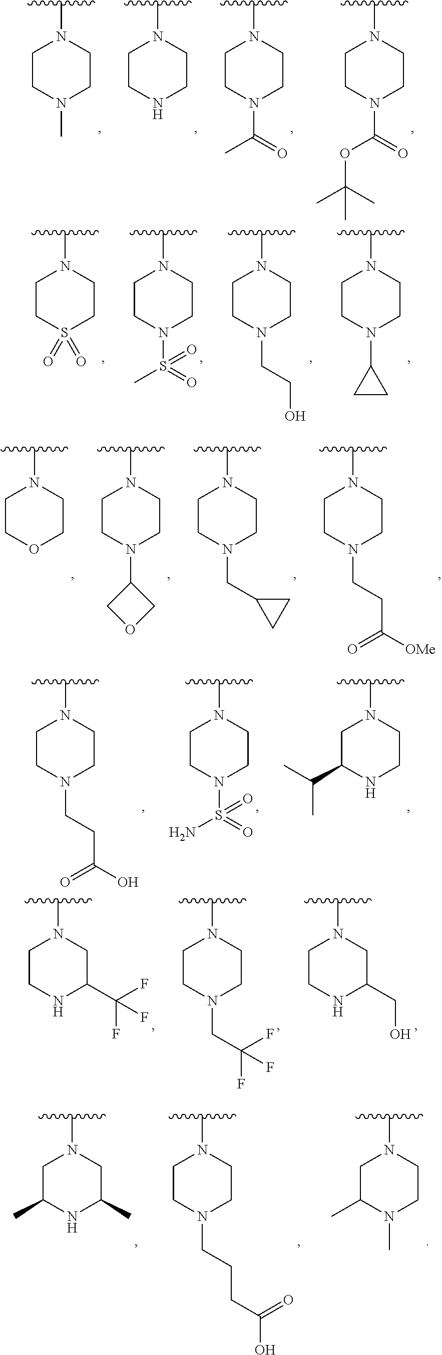

Further illustrative R⁵ groups include:

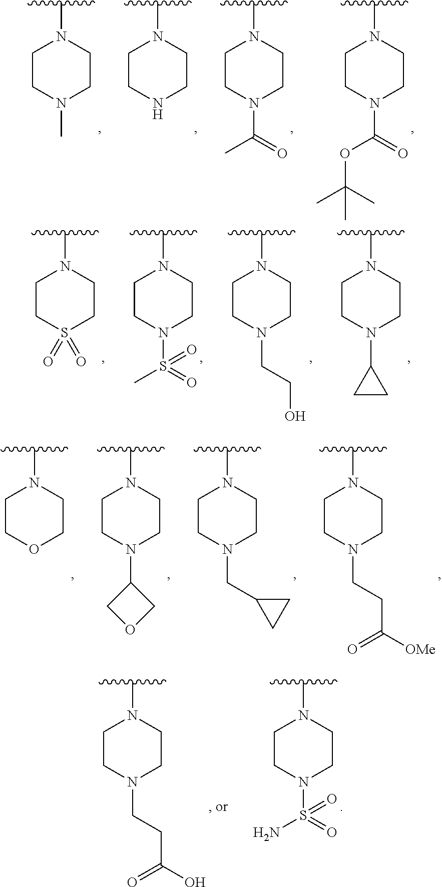

Further illustrative R⁵ groups include:

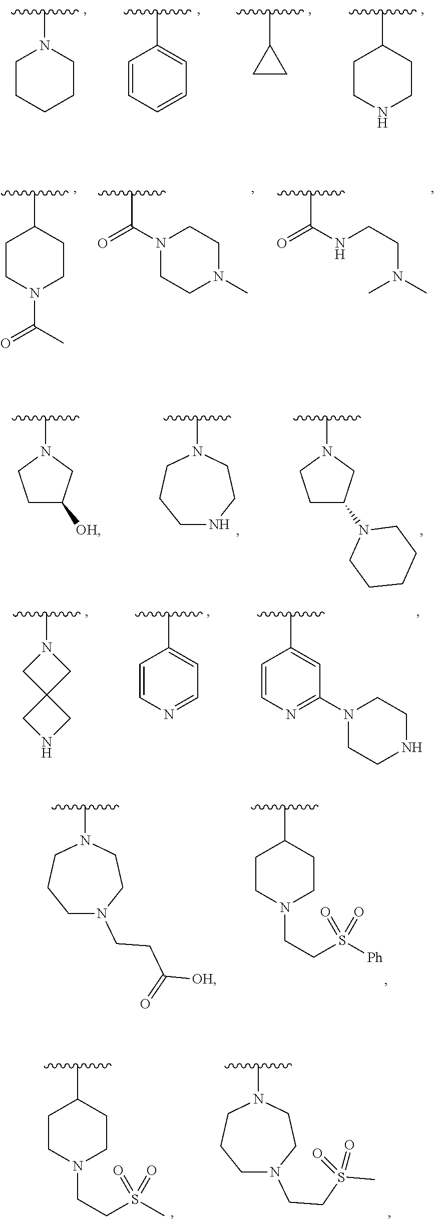

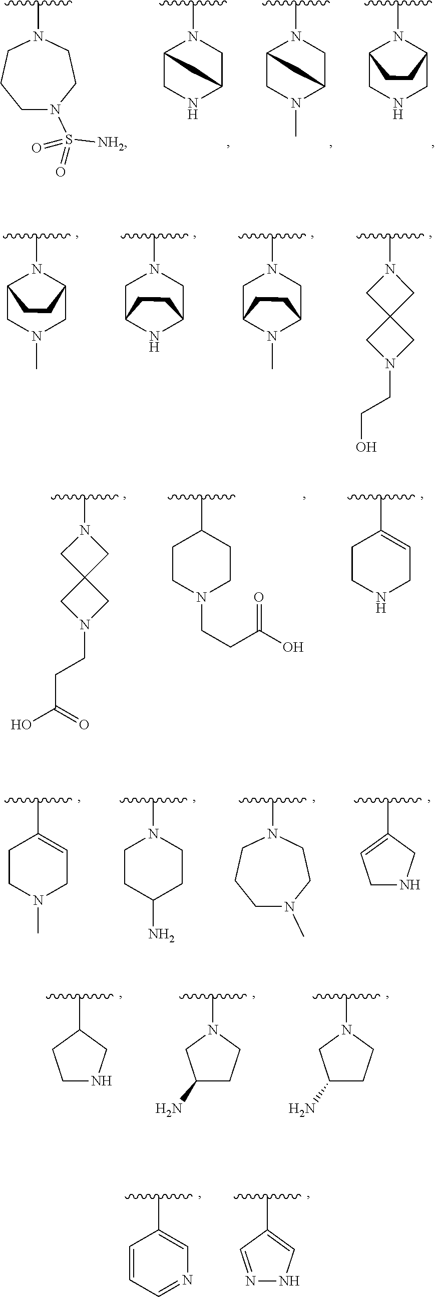

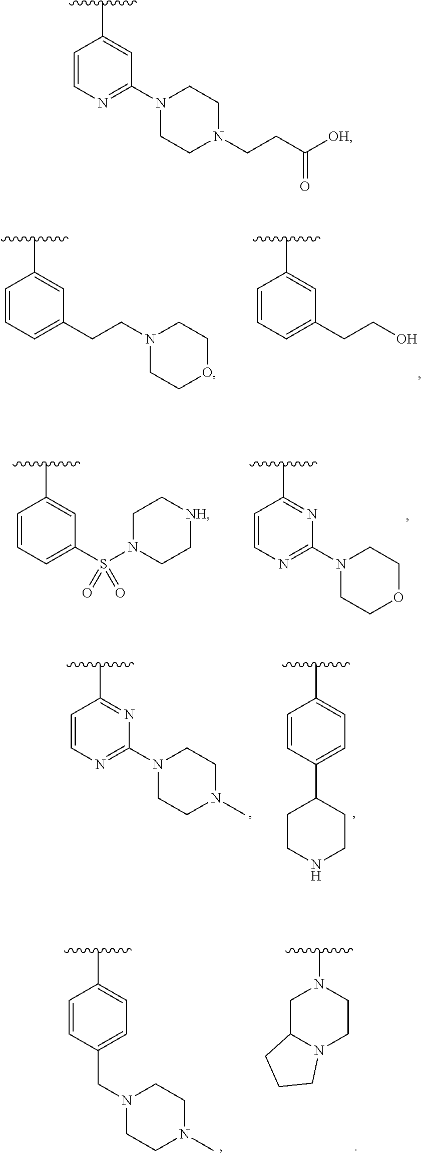

Further illustrative R⁵ groups include:

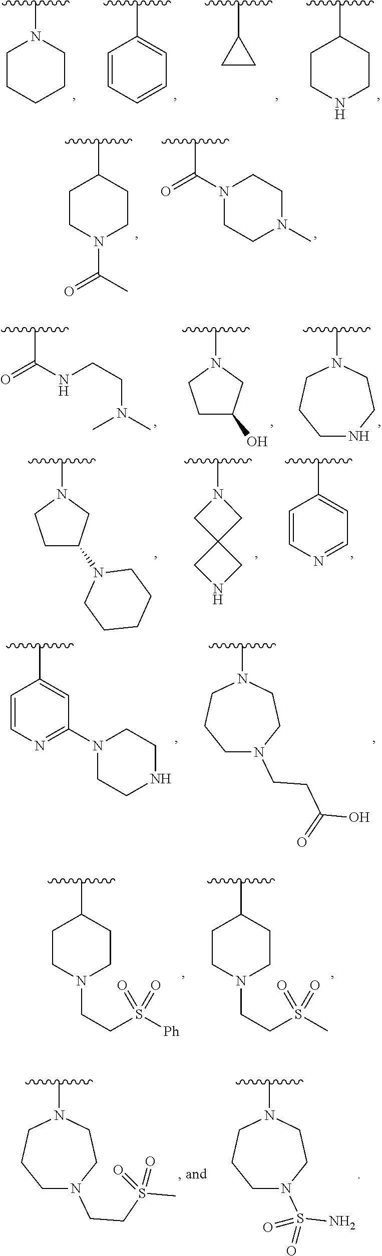

R⁶ may be H. R⁶ may be —C₁₋₆ alkyl, e.g. methyl, ethyl, propyl.

R⁷ may be independently selected at each occurrence from the group comprising: H and C₁-C₆-alkyl. It may be that R⁷ is H. It may be that R⁷ is —C₁₋₆ alkyl, e.g. methyl, ethyl, propyl.

R⁷ may be independently at each occurrence selected from the group comprising: halo, C₁-C₆-alkyl, C₁-C₆-haloalkyl, C₁-C₆-alkylene-R¹⁰, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₃₋₆ cycloalkyl, C₂₋₆-alkenyl, C₂₋₆-alkynyl, phenyl and 5- or 6-membered heteroaryl. R⁸ may be independently selected at each occurrence from the group comprising: halo, C₁-C₆-alkyl, C₁-C₆-haloalkyl, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₂₋₆-alkenyl, C₂₋₆-alkynyl, phenyl and 6-membered heteroaryl. R⁸ may be independently selected at each occurrence from the group comprising: halo, C₁-C₆-alkyl, C₁-C₆-haloalkyl, —OR¹⁰, cyano, nitro, —NR⁶R⁷, —SR¹⁰, C(O)R¹⁰, C(O)OR¹⁰, C(O)NR⁶R¹⁰, —S(O)R¹⁰, —S(O)₂R¹⁰, —S(O)₂NR⁶R¹⁰, C₂₋₆-alkenyl, C₂₋₆-alkynyl, 5- or 6-membered heterocycloalkyl, phenyl and 6-membered heteroaryl; wherein R⁸ is optionally substituted where chemically possible with one or more R^{8c} groups.

R⁸ may be independently selected at each occurrence from the group comprising: halo, C₁-C₆-alkyl, C₁-C₆-haloalkyl, —OR¹⁰, cyano, nitro, —NR⁶R⁷, —SR¹⁰, C(O)R¹⁰, C(O)OR¹⁰, C(O)NR⁶R¹⁰, —S(O)R¹⁰, —S(O)₂R¹⁰, —S(O)₂NR⁶R¹⁰, C₂₋₆-alkenyl, C₂₋₆-alkynyl, wherein R⁸ is optionally substituted where chemically possible with one or more R^{8c} groups. R⁸ may be independently selected at each occurrence from the group comprising: halo, C₁-C₆-alkyl, C₁-C₆-haloalkyl, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₂₋₆-alkenyl, C₂₋₆-alkynyl. R⁸ may be independently selected at each occurrence from the group comprising: halo, C₁-C₄-alkyl, C₁-C₄-haloalkyl, —OR¹⁰, cyano, nitro, —NR⁶R⁷, 5- or 6-membered heterocycloalkyl, phenyl and 5- or 6-membered heteroaryl; wherein R⁸ is optionally substituted where chemically possible with one or more R^{8c} groups. R⁸ may be independently selected at each occurrence from the group comprising: halo, C₁-C₄-alkyl, C₁-C₄-haloalkyl, —OR¹⁰, cyano, nitro, —NR⁶R⁷, phenyl and 6-membered heteroaryl. Re may be independently selected at each occurrence from the group comprising: halo, C₁-C₄-alkyl, C₁-C₄-haloalkyl, —OR¹⁰, cyano, and —NR⁶R⁷.

R^{8a} may be independently selected at each occurrence from the group comprising: halo, C₁-C₄-alkyl, C₁-C₄-haloalkyl, —OR¹⁰, cyano, and —NR⁶R⁷.

R^{8b} may be independently selected at each occurrence from the group comprising: halo, C₁-C₄-alkyl, C₁-C₄-haloalkyl, —OR^{a}, cyano, and —NR^{a}R^{b}.

R^{8c} may be independently selected at each occurrence from: halo, C₁-C₆-alkyl, C₁-C₆-haloalkyl, C₁-C₆-alkylene-R¹⁰, C₁-C₆-alkylene-NR⁶R¹⁰, —OR¹⁰, C(O)R¹⁰, C(O)OR¹⁰, C(O)NR⁶R¹⁰.

R⁹ may be independently at each occurrence selected from the group comprising: ═O, ═S, halo, C₁-C₆-alkyl, C₁-C₄-haloalkyl, —OR⁶, cyano, nitro, —NR⁶R⁷, —NR¹¹R¹², —SR⁶, C(O)R⁶, C(O)OR⁶, C(O)NR⁶R⁶, —S(O)R⁶, —S(O)₂R⁶, —S(O)₂NR⁶R⁶, C₃₋₆ cycloalkyl, 4-, 5- or 6-membered heterocycloalkyl, C₂₋₆-alkenyl C₂₋₆-alkynyl and C₁-C₃-alkylene-R^{9a}; wherein R^{9a} may be selected from OR⁶, SR⁶, S(O)₂R⁶, S(O)₂NR⁶R⁶, S(O)₂Ph, NR⁶R⁷, CO₂R⁶, CONR⁶R⁶, and cyclopropyl.

R⁹ may be independently at each occurrence selected from the group comprising: ═O, ═S, halo, C₁-C₆-alkyl, C₁-C₆-haloalkyl, —OR¹⁰, cyano, —NR⁶R⁷, —SR¹⁰, C(O)R¹⁰, C(O)OR¹⁰, C(O)NR⁶R¹⁰, —S(O)R¹⁰, —S(O)₂R¹⁰, —S(O)₂NR⁶R¹⁰, C₃₋₆ cycloalkyl, 4-, 5- or 6-membered heterocycloalkyl, and C₁-C₆-alkylene-R^{9a}. R⁹ may be independently at each occurrence selected from the group comprising: ═O, halo, C₁-C₄-alkyl, C₁-C₄-haloalkyl, —OR¹⁰, cyano, —NR⁶R⁷, —SR¹⁰, C(O)R¹⁰, C(O)OR¹⁰, C(O)NR⁶R¹⁰, —S(O)₂R¹⁰, —S(O)₂NR⁶R¹⁰, and C₁-C₃-alkylene-R^{9a}.

R⁹ may be independently at each occurrence selected from the group comprising: ═O, ═S, halo, C₁-C₆-alkyl, C₁-C₆-haloalkyl, —OR⁶, cyano, —NR⁶R⁷, —SR⁶, C(O)R⁶, C(O)OR⁶, C(O)NR⁶R⁶, —S(O)R⁶, —S(O)₂R⁶, —S(O)₂NR⁶R⁶, C₃₋₆ cycloalkyl, 4-, 5- or 6-membered heterocycloalkyl, and C₁-C₆-alkylene-R^{9a}. R⁹ may be independently at each occurrence selected from the group comprising: ═O, halo, C₁-C₄-alkyl, C₁-C₄-haloalkyl, —OR⁶, cyano, —NR⁶R⁷, —SR⁶, C(O)R⁶, C(O)OR⁶, C(O)NR⁶R⁶, —S(O)₂R⁶, —S(O)₂NR⁶R⁶, and C₁-C₃-alkylene-R^{9a}.

R^{9a} may be independently selected at each occurrence from OR⁶, S(O)₂R⁶, S(O)₂Ph, NR⁶R⁷, CO₂R, CONR⁶R⁶, 4-, 5- or 6-membered heterocycloalkyl, and cyclopropyl. R^{9a} may be independently selected at each occurrence from OR⁶, S(O)₂R⁶, S(O)₂Ph, CO₂R⁶ and cyclopropyl.

R^{9b} may be independently selected at each occurrence from OR⁶, S(O)₂R⁶, S(O)₂Ph, NR⁶R⁷, CO₂R⁶, CONR⁶R⁶, and cyclopropyl. R^{9a} may be independently selected at each occurrence from OR⁶, S(O)₂R⁶, S(O)₂Ph, CO₂R⁶ and cyclopropyl.

R^{9b} may be independently at each occurrence selected from the group comprising: H, C₁-C₆-alkyl, C₁-C₆-haloalkyl, C(O)R¹⁰, C(O)OR¹⁰, C(O)NR⁶R¹⁰, —S(O)R¹⁰, —S(O)₂R¹⁰, —S(O)₂NR⁶R¹⁰, C₃₋₆ cycloalkyl, 4-, 5- or 6-membered heterocycloalkyl, C₂₋₆-alkenyl C₂₋₆-alkynyl, C₂-C₃-alkylene-R^{9a} and CH₂-cyclopropyl. R^{9b} may be independently at each occurrence selected from the group comprising: H, C₁-C₄-alkyl, C(O)R¹⁰, C(O)OR¹⁰, —S(O)₂R¹⁰, —S(O)₂NR⁶R¹⁰, C₃₋₆ cycloalkyl, C₂-C₃-alkylene-R^{9a} and CH₂-cyclopropyl. R^{9b} may be selected from the group comprising: H, C₁₋₄ alkyl, C(O)R¹⁰, C₂-C₃-alkylene-R^{9a} and CH₂-cyclopropyl. R^{9b} may be H. R^{9b} may be C₁-C₄-alkyl, e.g. methyl, ethyl, propyl. R^{9b} may be C(O)R¹⁰, e.g. C(O)Me, C(O)Et. R^{9b} may be C₂-C₃-alkylene-R^{9a}, e.g. CH₂CH₂R^{9a}, CH₂CH₂CH₂R^{9a}. R^{9b} may be CH₂-cyclopropyl.

R^{9b} may be independently at each occurrence selected from the group comprising: H, C₁-C₆-alkyl, C₁-C₆-haloalkyl, C(O)R⁶, C(O)OR⁶, C(O)NR⁶R⁶, —S(O)R⁶, —S(O)₂R⁶, —S(O)₂NR⁶R⁶, C₃₋₆ cycloalkyl, 4-, 5- or 6-membered heterocycloalkyl, C₂₋₆-alkenyl C₂₋₆-alkynyl, C₂-C₃-alkylene-R^{9a} and CH₂-cyclopropyl. R^{9b} may be independently at each occurrence selected from the group comprising: H, C₁-C₄-alkyl, C(O)R⁶, C(O)OR⁶, —S(O)₂R⁶, —S(O)₂NR⁶R⁶, C₃₋₆ cycloalkyl, C₂-C₃-alkylene-R^{9a} and CH₂-cyclopropyl. R^{9b} may be selected from the group comprising: H, C₁₋₄ alkyl, C(O)R⁶, C₂-C₃-alkylene-R^{9a} and CH₂-cyclopropyl. R^{9b} may be H. R^{9b} may be C₁-C₄-alkyl, e.g. methyl, ethyl, propyl. R^{9b} may be C(O)R⁶, e.g. C(O)Me, C(O)Et. R^{9b} may be C₂-C₃-alkylene-R^{9a}, e.g. CH₂CH₂R^{9a}, CH₂CH₂CH₂R^{9a}. R^{9b} may be CH₂-cyclopropyl.

R^{9b} may be independently at each occurrence selected from the group comprising: C₁-C₆-alkyl, C₁-C₆-haloalkyl, C(O)R¹⁰, C(O)OR¹⁰, C(O)NR⁶R¹⁰, —S(O)R¹⁰, —S(O)₂R¹⁰, —S(O)₂NR⁶R¹⁰, C₃₋₆ cycloalkyl, 4-, 5- or 6-membered heterocycloalkyl, C₂₋₆-alkenyl C₂₋₆-alkynyl, C₂-C₃-alkylene-R^{9a} and CH₂-cyclopropyl. R^{9b} may be independently at each occurrence selected from the group comprising: C₁-C₄-alkyl, C(O)R¹⁰, C(O)OR¹⁰, —S(O)₂R¹⁰, —S(O)₂NR⁶R¹⁰, C₃₋₆ cycloalkyl, C₂-C₃-alkylene-R^{9a} and CH₂-cyclopropyl. R^{9b} may be selected from the group comprising: C₁₋₄ alkyl, C(O)R¹⁰, C₂-C₃-alkylene-R^{9a} and CH₂-cyclopropyl. R^{9b} may be C₁-C₄-alkyl, e.g. methyl, ethyl, propyl. R^{9b} may be C(O)R¹⁰, e.g. C(O)Me, C(O)Et. R^{9b} may be C₂-C₃-alkylene-R^{9a}, e.g. CH₂CH₂R^{9a}, CH₂CH₂CH₂R^{9a}. R^{9b} may be CH₂-cyclopropyl.

R^{9b} may be independently at each occurrence selected from the group comprising: H, C₁-C₆-alkyl, C₁-C₆-haloalkyl, C(O)R¹⁰, C(O)OR¹⁰, C(O)NR⁶R¹⁰, —S(O)R¹⁰, —S(O)₂R¹⁰, —S(O)₂NR⁶R¹⁰, C₃₋₆ cycloalkyl, 4-, 5- or 6-membered heterocycloalkyl, C₂₋₆-alkenyl C₂₋₆-alkynyl and C₂-C₃-alkylene-R^{9a}. R^{9b} may be independently at each occurrence selected from the group comprising: H, C₁-C₄-alkyl, C(O)R¹⁰, C(O)OR¹⁰, —S(O)₂R¹⁰, —S(O)₂NR⁶R¹⁰, C₃₋₆ cycloalkyl, and C₂-C₃-alkylene-R^{9a}. R^{9b} may be selected from the group comprising: H, C₁₋₄ alkyl, C(O)R¹⁰, and C₂-C₃-alkylene-R^{9a}. R^{9b} may be H. R^{9b} may be C₁-C₄-alkyl, e.g. methyl, ethyl, propyl. R^{9b} may be C(O)R¹⁰, e.g. C(O)Me, C(O)Et. R^{9b} may be C₂-C₃-alkylene-R^{9a}, e.g. CH₂CH₂R^{9a}, CH₂CH₂CH₂R9^{a}.

R^{9b} may be independently at each occurrence selected from the group comprising: H, C₁-C₆-alkyl, C₁-C₆-haloalkyl, C(O)R⁶, C(O)OR⁶, C(O)NR⁶R⁶, —S(O)R⁶, —S(O)₂R⁶, —S(O)₂NR⁶R⁶, C₃₋₆ cycloalkyl, 4-, 5- or 6-membered heterocycloalkyl, C₂₋₆-alkenyl C₂₋₆-alkynyl and C₂-C₃-alkylene-R^{9a}. R^{9b} may be independently at each occurrence selected from the group comprising: H, C₁-C₄-alkyl, C(O)R⁶, C(O)OR⁶, —S(O)₂R⁶, —S(O)₂NR⁶R⁶, C₃₋₆ cycloalkyl, and C₂-C₃-alkylene-R^{9a}. R^{9b} may be selected from the group comprising: H, C₁₋₄ alkyl, C(O)R⁶, and C₂-C₃-alkylene-R^{9a}. R^{9b} may be H. R^{9b} may be C₁-C₄-alkyl, e.g. methyl, ethyl, propyl. R^{9b} may be C(O)R⁶, e.g. C(O)Me, C(O)Et. R^{9b} may be C₂-C₃-alkylene-R^{9a}, e.g. CH₂CH₂R^{9a}, CH₂CH₂CH₂R9^{a}.

R^{9b} may be independently at each occurrence selected from the group comprising: C₁-C₆-alkyl, C₁-C₆-haloalkyl, C(O)R¹⁰, C(O)OR¹⁰, C(O)NR⁶R¹⁰, —S(O)R¹⁰, —S(O)₂R¹⁰, —S(O)₂NR⁶R¹⁰, C₃₋₆ cycloalkyl, 4-, 5- or 6-membered heterocycloalkyl, C₂₋₆-alkenyl C₂₋₆-alkynyl and C₂-C₃-alkylene-R^{9a}. R^{9b} may be independently at each occurrence selected from the group comprising: C₁-C₄-alkyl, C(O)R¹⁰, C(O)OR¹⁰, —S(O)₂R¹⁰, —S(O)₂NR⁶R¹⁰, C₃₋₆ cycloalkyl, and C₂-C₃-alkylene-R^{9a}. R^{9b} may be selected from the group comprising: C₁₋₄ alkyl, C(O)R¹⁰, and C₂-C₃-alkylene-R^{9a}. R^{9b} may be C₁-C₄-alkyl, e.g. methyl, ethyl, propyl. R^{9b} may be C(O)R¹⁰, e.g. C(O)Me, C(O)Et. R^{9b} may be C₂-C₃-alkylene-R^{9a}, e.g. CH₂CH₂R^{9a}, CH₂CH₂CH₂R9^{a}.

R¹⁰ may be independently selected at each occurrence from the group comprising: H, C₁-C₆-alkyl, C₁-C₆-haloalkyl, C₁-C₆-alkylene-R^{10a}, C₃₋₈ cycloalkyl, and 4-, 5-, 6-, 7- or 8-membered heterocycloalkyl. R¹⁰ may be independently selected at each occurrence from the group comprising: H, C₁-C₆-alkyl, C₁-C₆-haloalkyl, and C₁-C₆-alkylene-R^{10a}. R¹⁰ may be independently selected at each occurrence from the group comprising: H, C₁-C₄-alkyl, and C₁-C₆-alkylene-R^{10a}. It may be that R¹⁰ is H. It may be that R¹⁰ is —C₁₋₄ alkyl, e.g. methyl, ethyl, propyl. R¹⁰ may be C₁-C₃-alkylene-R^{10a}, e.g. —CH₂R^{10a}, —CH₂CH₂R^{10a} or —CH₂CH₂CH₂R^{10a}.

R^{10a} may be independently selected at each occurrence from cyclopropyl, OR⁶, S(O)₂R⁶, NR⁶R⁷, CO₂R⁶, CONR⁶R⁶, phenyl, 5- or 6-membered heteroaryl, and 5- or 6-membered heterocycloalkyl. R^{10a} may be independently selected at each occurrence from cyclopropyl, OR⁶, S(O)₂R⁶, NR⁶R⁷, CO₂R⁶ and CONR⁶R⁶. R^{10a} may be independently selected at each occurrence from OR⁶, NR⁶R⁷, and CO₂R⁶.

R¹¹ may be H. R¹¹ may be —C₁₋₆ alkyl, e.g. methyl, ethyl, propyl.

R¹² may be selected from the group comprising: piperidyl, piperazyl, morpholinyl, and tetrahydropyran, optionally substituted with at least one R¹³ group. It may be that R¹² is piperidyl or piperazyl, optionally substituted with at least one R¹³ group.

R¹³ may be independently at each occurrence selected from: ═O, ═S, halo, C₁-C₆-alkyl, C₁-C₄-haloalkyl, —OR⁶, cyano, nitro, —NR⁶R⁷, —SR⁶, C(O)R⁶, C(O)OR⁶, C(O)NR⁶R⁶, —S(O)R⁶, —S(O)₂R⁶, —S(O)₂NR⁶R⁶, and C₁-C₆-alkylene-R¹³; wherein R¹³ is selected from OR⁶, SR⁶, S(O)₂R⁶, S(O)₂Ph, NR⁶R⁷, CO₂R⁶ and CONR⁶R⁶. R¹³ may be independently at each occurrence selected from: ═O, halo, C₁-C₄-alkyl, C₁-C₄-haloalkyl, —OR⁶, cyano, —NR⁶R⁷, C(O)R⁶, C(O)OR⁶, and C(O)NR⁶R⁶.

R¹⁴ may be H. R¹⁴ may be C₁-C₃-alkylene-R^{14a}, e.g. —CH₂R^{14a}, —CH₂CH₂R^{14a} or —CH₂CH₂CH₂R^{14a}.

R^{14a} may be selected from OR⁶, S(O)₂R⁶, NR⁶R⁷, CO₂R⁶ and CONR⁶R⁶. R^{14a} may be selected from OR⁶, NR⁶R⁷, and CO₂R⁶. R^{14a} may be OR⁶, e.g. OH or OMe. R^{14a} may be NR⁶R⁷, e.g. NH₂, NHMe or NMe₂. R^{14a} may be CO₂R⁶, e.g. C(O)OH, C(O)OMe or C(O)OEt.

m may be 0. m may be an integer selected from 1, 2, 3, 4, 5, 6, and 7. m may be an integer selected from 0, 1, 2, 3, and 4. m may be an integer selected from 0, 1, and 2. Preferably, however, m is 0 or 1.

n may be 0. Preferably, however, n is an integer selected from 1, 2, 3 and 4. n may be an integer selected from 1, 2 and 3. n may be 0 or 1. n may be 1.

n1 may be 0. n1 may be an integer selected from 1 and 2. n1 may be 1.

p may be 0. p may be an integer selected from 1, 2, 3, 4, and 5. p may be an integer selected from 0, 1, and 2. Preferably, however, p is 0 or 1.

q may be 0. q may be an integer selected from 1, 2, 3, and 4. q may be an integer selected from 0, 1, and 2. Preferably, however, q is 0 or 1.

x may be 0, 1, 2 or 3. x may be 0. x may be 1. x may be 2. x may be 3.

y may be 0, 1, 2 or 3. y may be 0. y may be 1. y may be 2. y may be 3.

The compounds of formula (I) may be selected from:

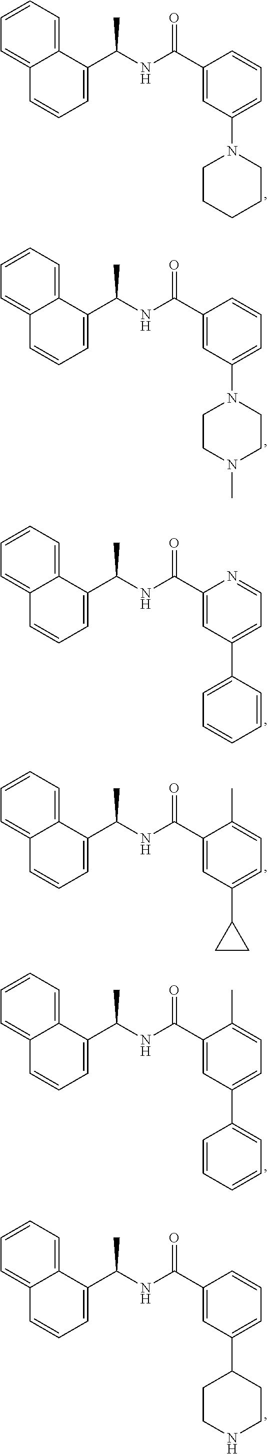

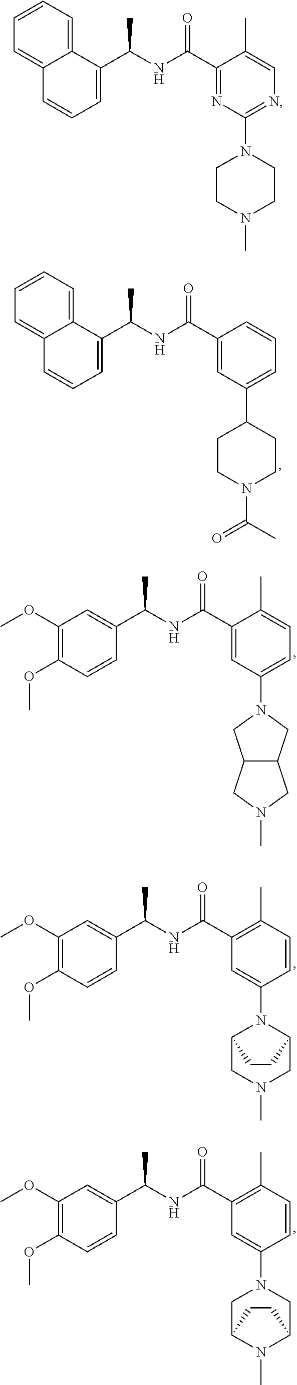

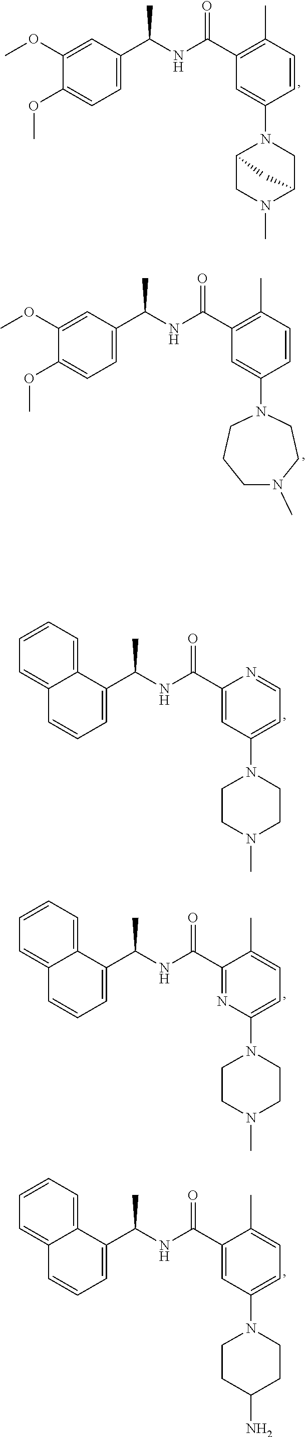

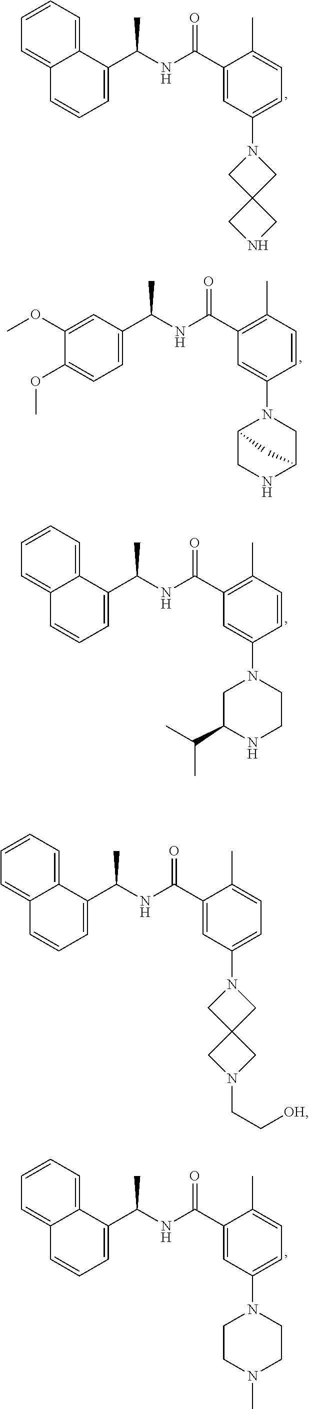

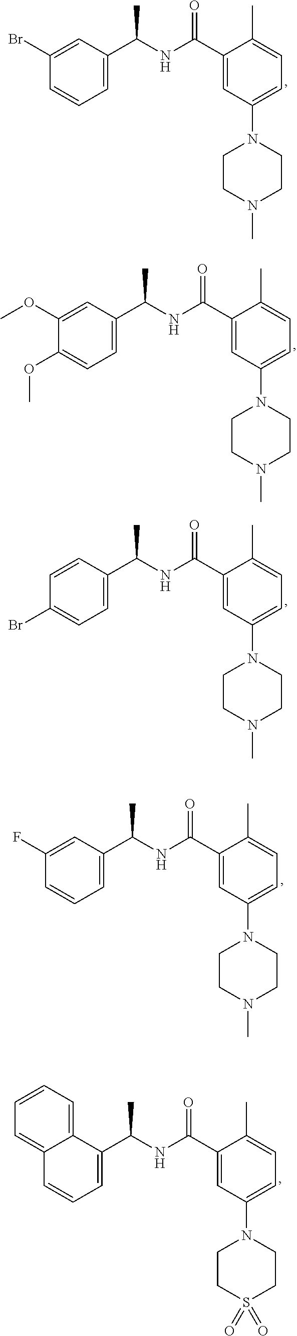

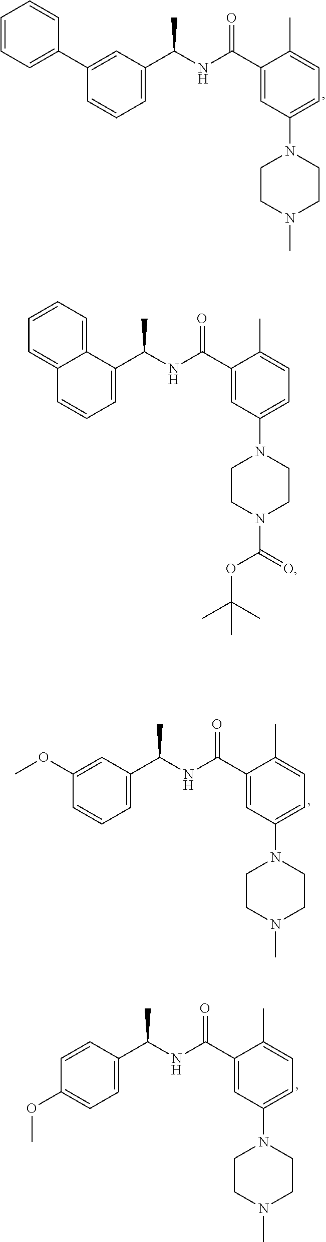 [SMILES: Cc1ccc(N2CCN(C)CC2)cc1C(=O)N[C@H](C)c1cccc(-c2ccccc2)c1 ; Cc1ccc(N2CCN(C(=O)OC(C)(C)C)CC2)cc1C(=O)N[C@H](C)c1cccc2ccccc12 ; COc1cccc([C@@H](C)NC(=O)c2cc(N3CCN(C)CC3)ccc2C)c1 ; COc1ccc([C@@H](C)NC(=O)c2cc(N3CCN(C)CC3)ccc2C)cc1]

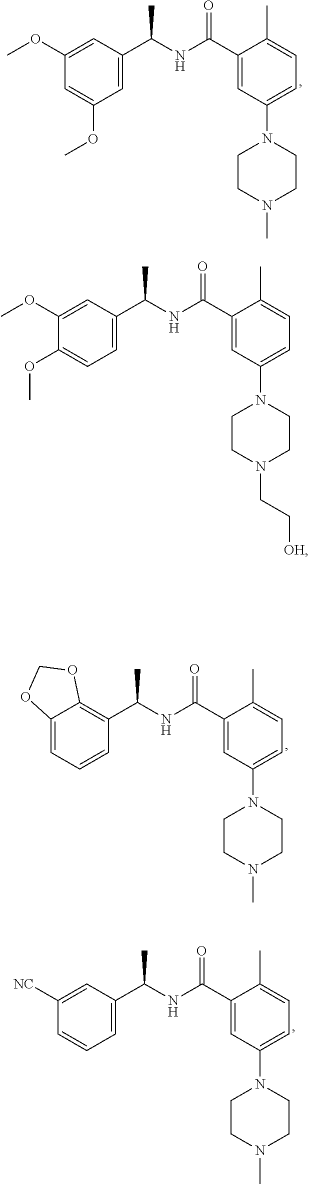

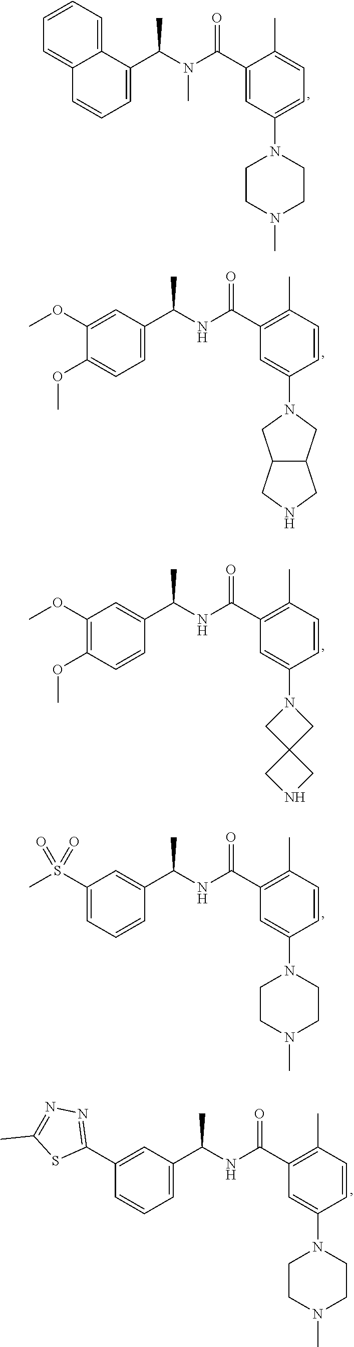

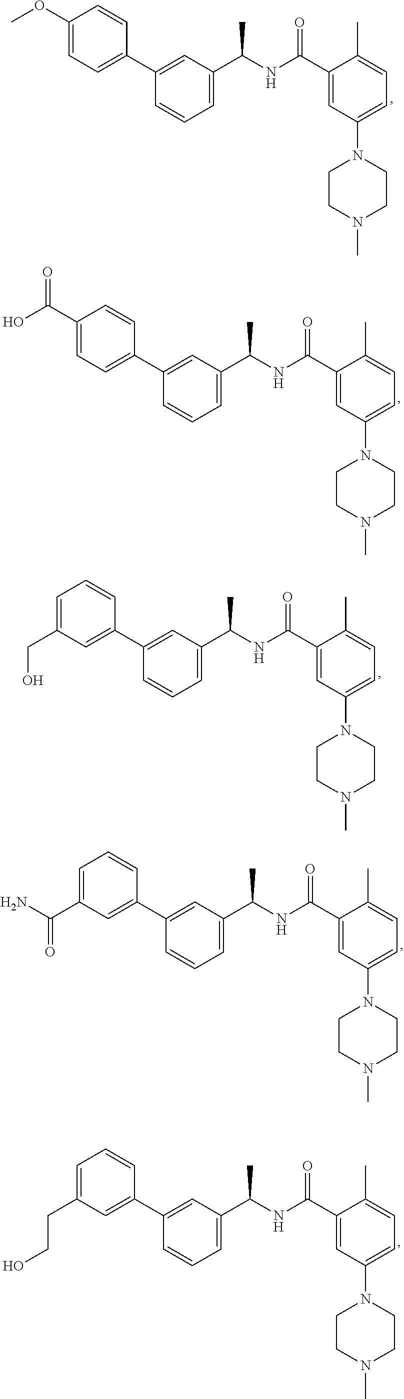

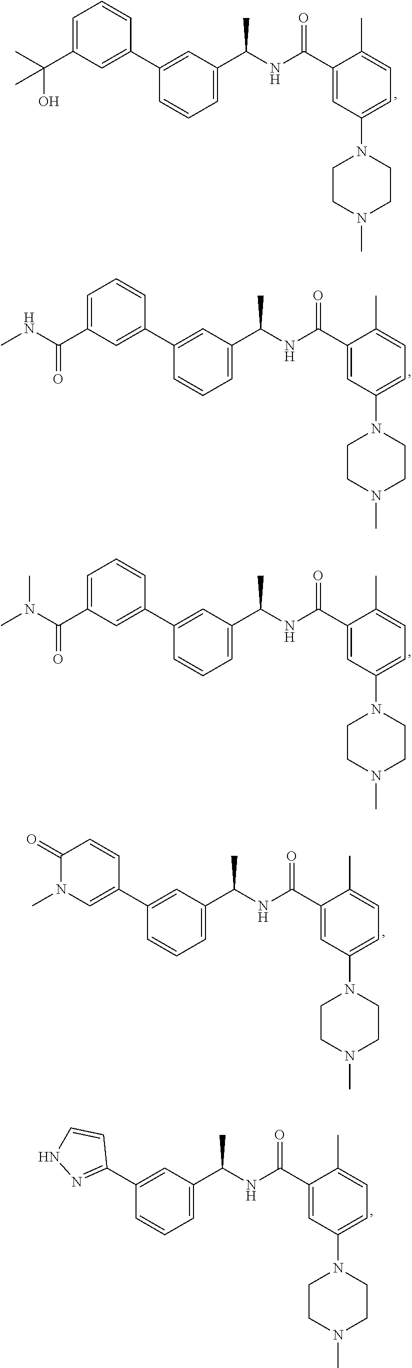

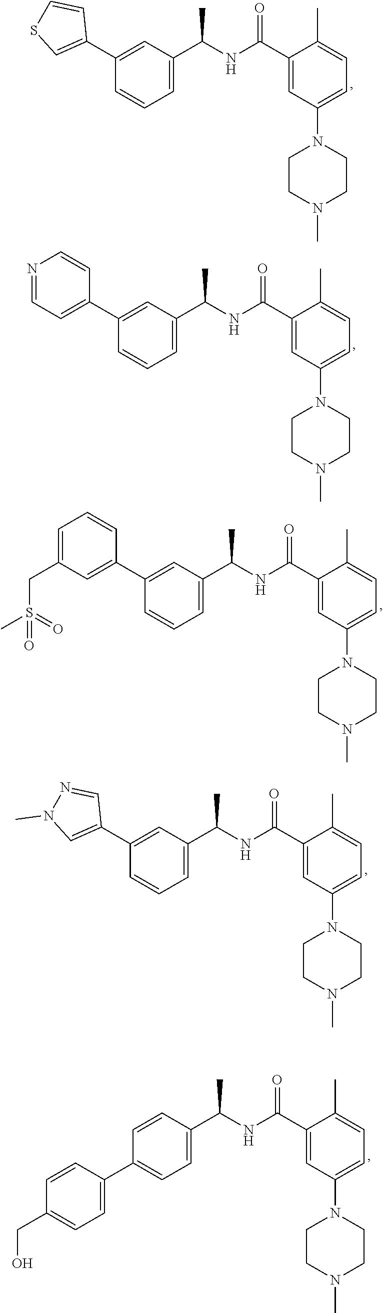

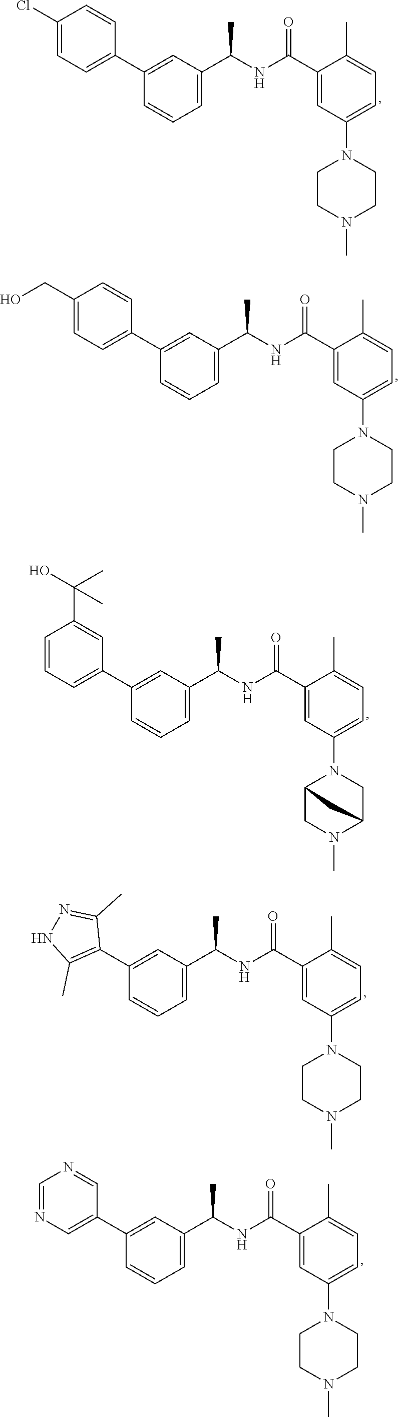

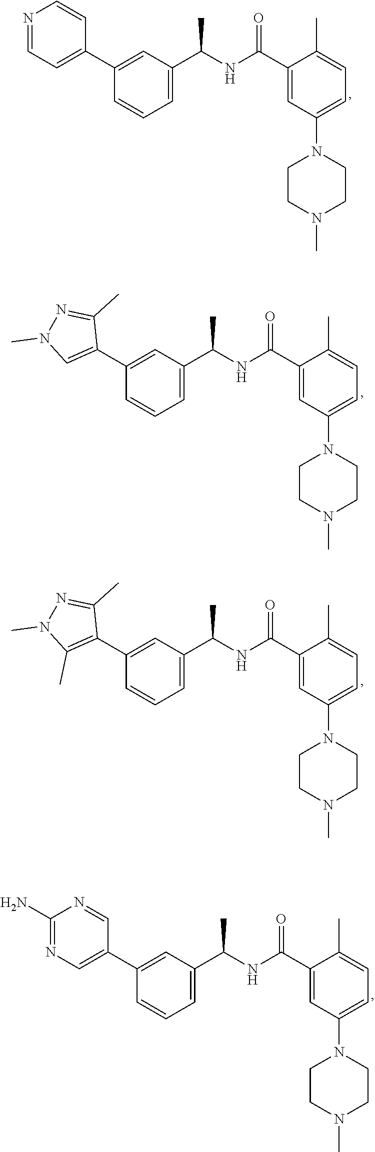 [SMILES: Cc1ccc(N2CCN(C)CC2)cc1C(=O)N[C@H](C)c1cccc(-c2ccncc2)c1 ; Cc1ccc(N2CCN(C)CC2)cc1C(=O)N[C@H](C)c1cccc(-c2cn(C)nc2C)c1 ; Cc1ccc(N2CCN(C)CC2)cc1C(=O)N[C@H](C)c1cccc(-c2c(C)nn(C)c2C)c1 ; Cc1ccc(N2CCN(C)CC2)cc1C(=O)N[C@H](C)c1cccc(-c2cnc(N)nc2)c1]

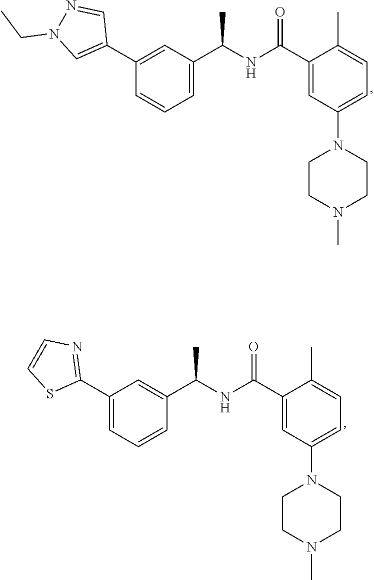 [SMILES: CCn1cc(-c2cccc([C@@H](C)NC(=O)c3cc(N4CCN(C)CC4)ccc3C)c2)cn1 ; Cc1ccc(N2CCN(C)CC2)cc1C(=O)N[C@H](C)c1cccc(-c2nccs2)c1]

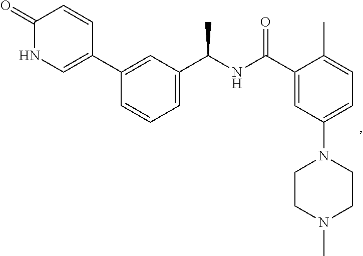 [SMILES: Cc1ccc(N2CCN(C)CC2)cc1C(=O)N[C@H](C)c1cccc(-c2ccc(=O)[nH]c2)c1]

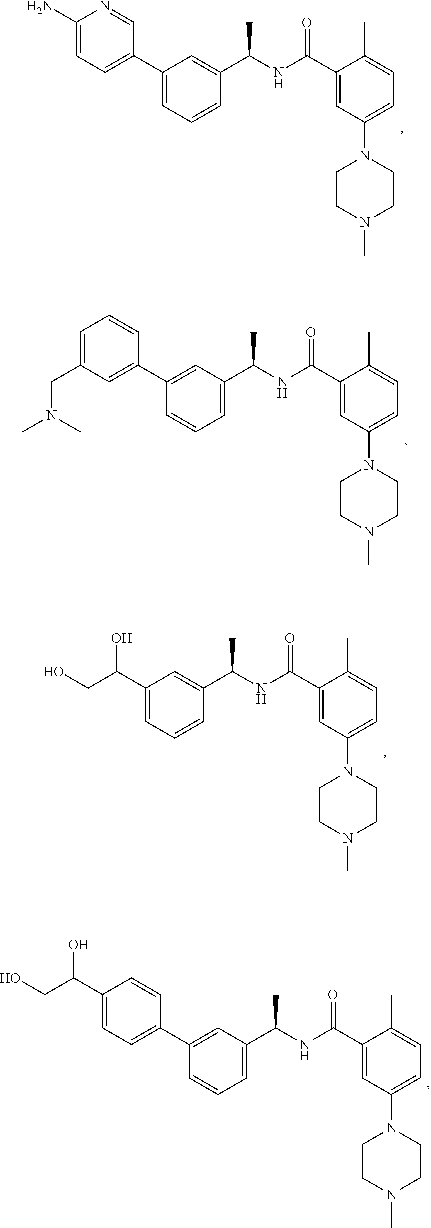 [SMILES: Cc1ccc(N2CCN(C)CC2)cc1C(=O)N[C@H](C)c1cccc(-c2ccc(N)nc2)c1 ; Cc1ccc(N2CCN(C)CC2)cc1C(=O)N[C@H](C)c1cccc(-c2cccc(CN(C)C)c2)c1 ; Cc1ccc(N2CCN(C)CC2)cc1C(=O)N[C@H](C)c1cccc(C(O)CO)c1 ; Cc1ccc(N2CCN(C)CC2)cc1C(=O)N[C@H](C)c1cccc(-c2ccc(C(O)CO)cc2)c1]

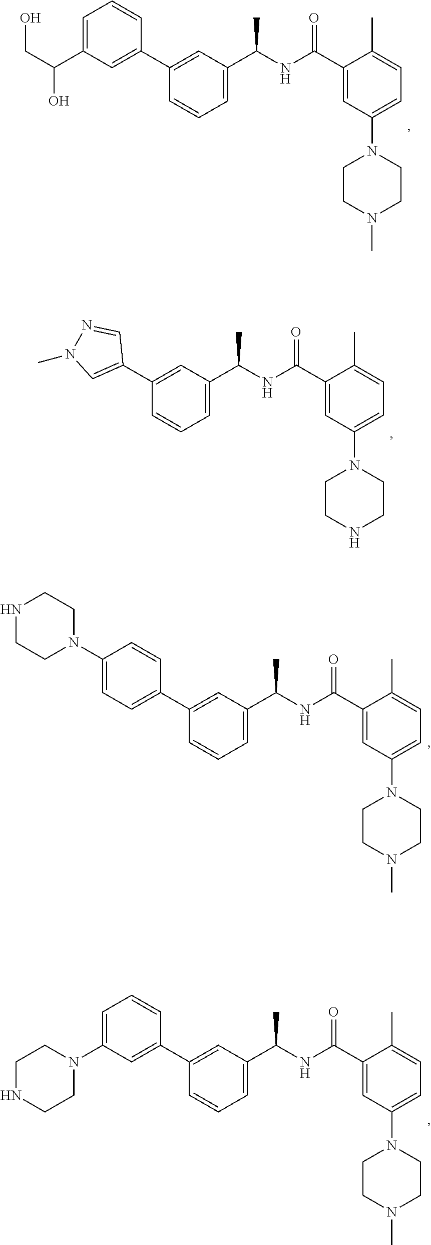 [SMILES: Cc1ccc(N2CCN(C)CC2)cc1C(=O)N[C@H](C)c1cccc(-c2cccc(C(O)CO)c2)c1 ; Cc1ccc(N2CCNCC2)cc1C(=O)N[C@H](C)c1cccc(-c2cnn(C)c2)c1 ; Cc1ccc(N2CCN(C)CC2)cc1C(=O)N[C@H](C)c1cccc(-c2ccc(N3CCNCC3)cc2)c1 ; Cc1ccc(N2CCN(C)CC2)cc1C(=O)N[C@H](C)c1cccc(-c2cccc(N3CCNCC3)c2)c1]

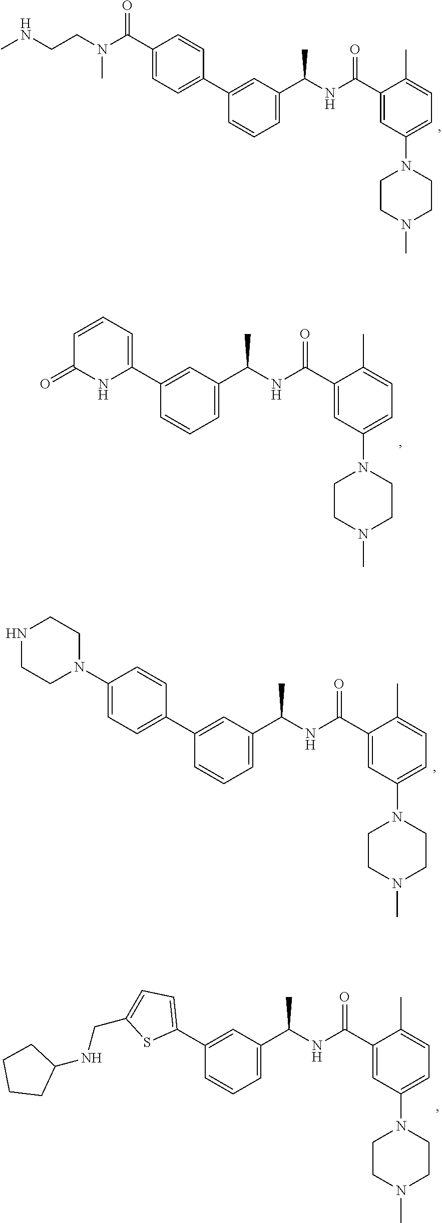 [SMILES: CNCCN(C)C(=O)c1ccc(-c2cccc([C@@H](C)NC(=O)c3cc(N4CCN(C)CC4)ccc3C)c2)cc1 ; Cc1ccc(N2CCN(C)CC2)cc1C(=O)N[C@H](C)c1cccc(-c2cccc(=O)[nH]2)c1 ; Cc1ccc(N2CCN(C)CC2)cc1C(=O)N[C@H](C)c1cccc(-c2ccc(N3CCNCC3)cc2)c1 ; Cc1ccc(N2CCN(C)CC2)cc1C(=O)N[C@H](C)c1cccc(-c2ccc(CNC3CCCC3)s2)c1]

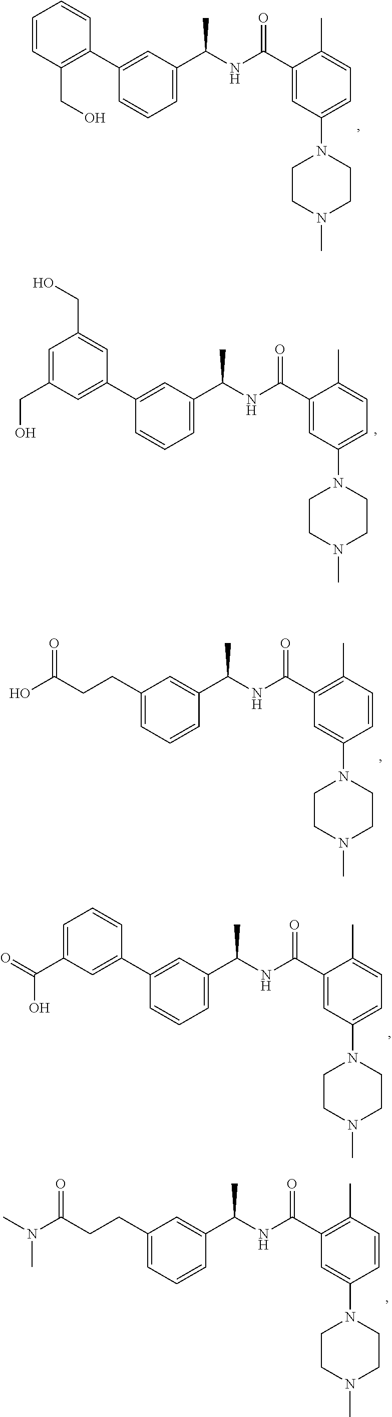

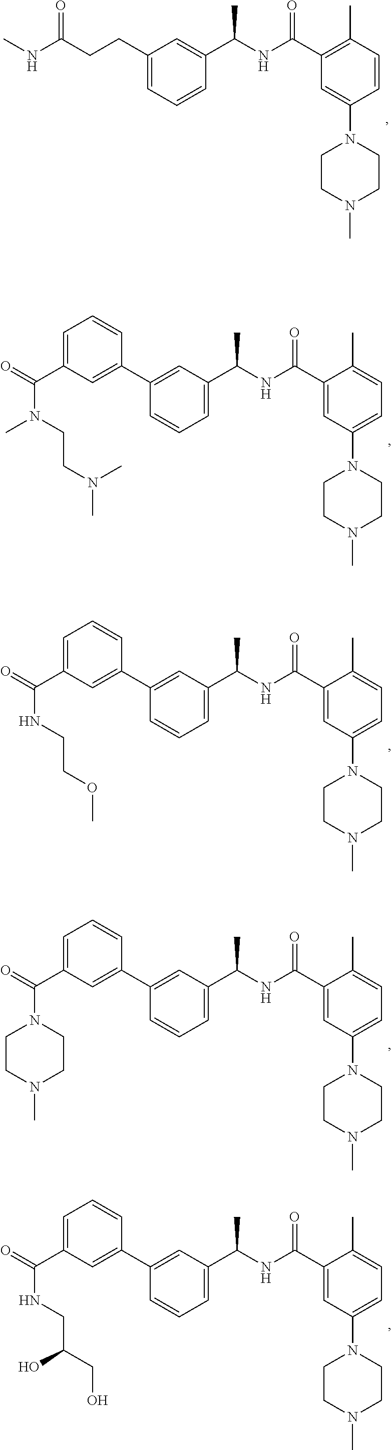

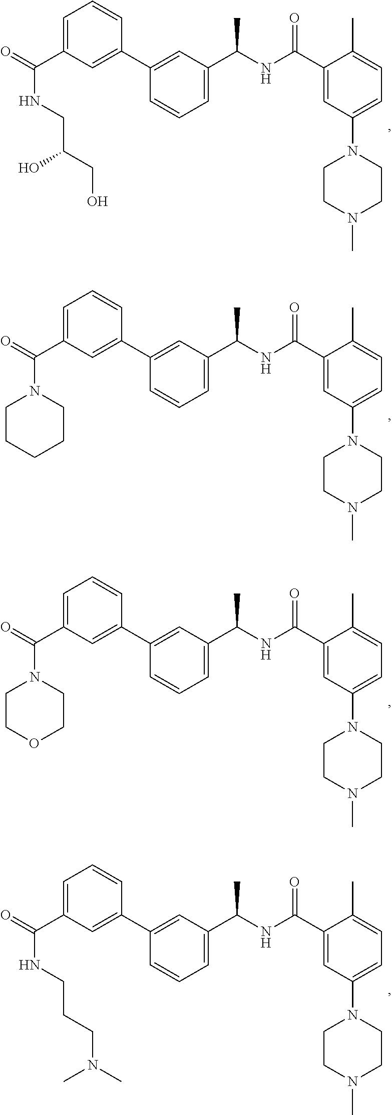 [SMILES: Cc1ccc(N2CCN(C)CC2)cc1C(=O)N[C@H](C)c1cccc(-c2cccc(C(=O)NC[C@@H](O)CO)c2)c1 ; Cc1ccc(N2CCN(C)CC2)cc1C(=O)N[C@H](C)c1cccc(-c2cccc(C(=O)N3CCCCC3)c2)c1 ; Cc1ccc(N2CCN(C)CC2)cc1C(=O)N[C@H](C)c1cccc(-c2cccc(C(=O)N3CCOCC3)c2)c1 ; Cc1ccc(N2CCN(C)CC2)cc1C(=O)N[C@H](C)c1cccc(-c2cccc(C(=O)NCCCN(C)C)c2)c1]

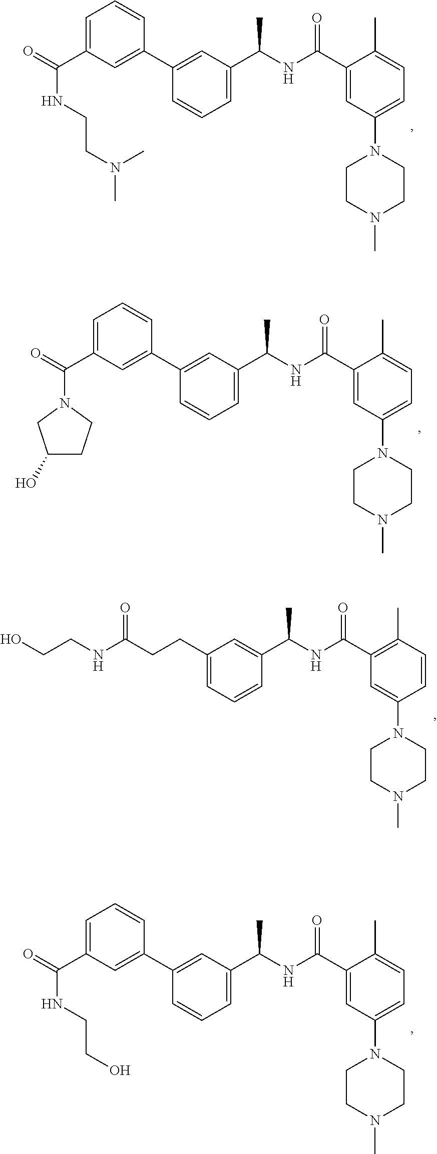 [SMILES: Cc1ccc(N2CCN(C)CC2)cc1C(=O)N[C@H](C)c1cccc(-c2cccc(C(=O)NCCN(C)C)c2)c1 ; Cc1ccc(N2CCN(C)CC2)cc1C(=O)N[C@H](C)c1cccc(-c2cccc(C(=O)N3CC[C@H](O)C3)c2)c1 ; Cc1ccc(N2CCN(C)CC2)cc1C(=O)N[C@H](C)c1cccc(CCC(=O)NCCO)c1 ; Cc1ccc(N2CCN(C)CC2)cc1C(=O)N[C@H](C)c1cccc(-c2cccc(C(=O)NCCO)c2)c1]

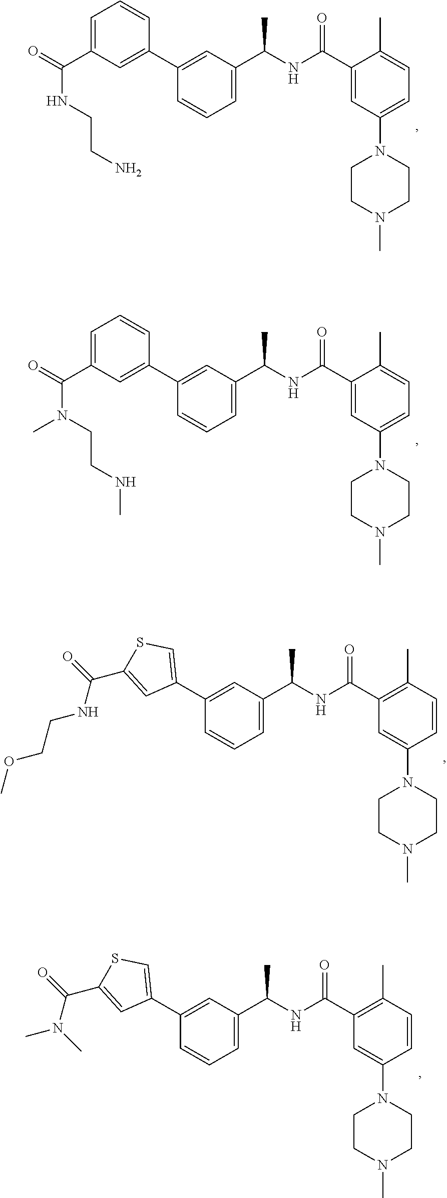 [SMILES: Cc1ccc(N2CCN(C)CC2)cc1C(=O)N[C@H](C)c1cccc(-c2cccc(C(=O)NCCN)c2)c1 ; CNCCN(C)C(=O)c1cccc(-c2cccc([C@@H](C)NC(=O)c3cc(N4CCN(C)CC4)ccc3C)c2)c1 ; COCCNC(=O)c1cc(-c2cccc([C@@H](C)NC(=O)c3cc(N4CCN(C)CC4)ccc3C)c2)cs1 ; Cc1ccc(N2CCN(C)CC2)cc1C(=O)N[C@H](C)c1cccc(-c2csc(C(=O)N(C)C)c2)c1]

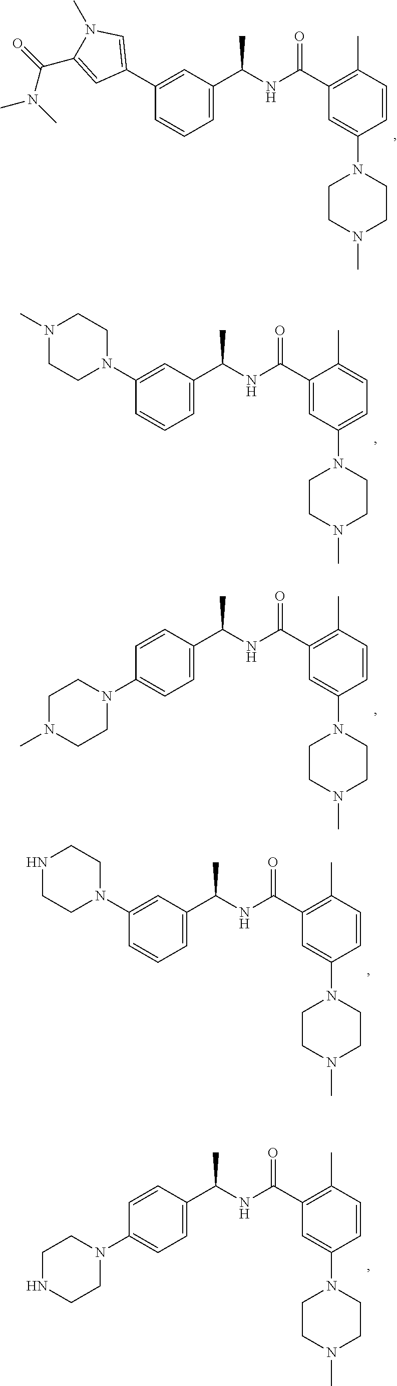

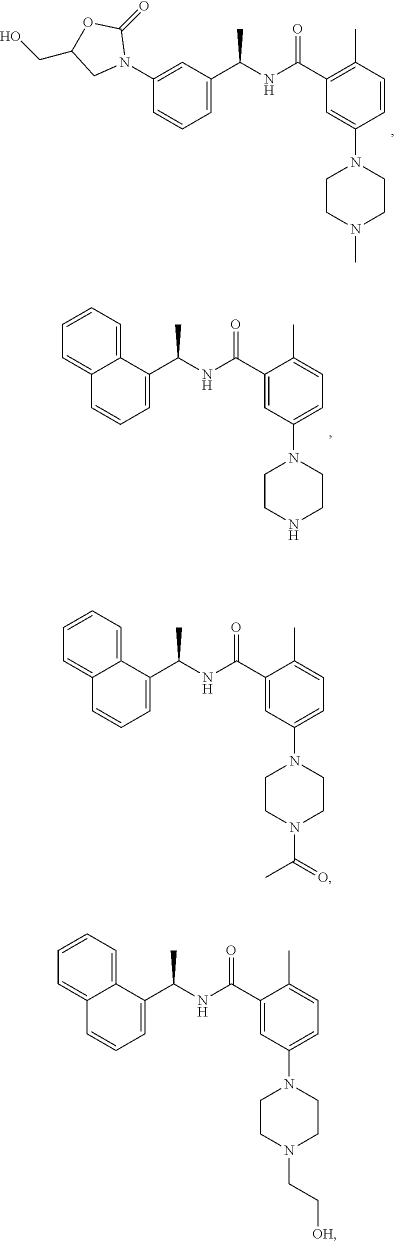 [SMILES: Cc1ccc(N2CCN(C)CC2)cc1C(=O)N[C@H](C)c1cccc(N2CC(CO)OC2=O)c1 ; Cc1ccc(N2CCNCC2)cc1C(=O)N[C@H](C)c1cccc2ccccc12 ; CC(=O)N1CCN(c2ccc(C)c(C(=O)N[C@H](C)c3cccc4ccccc34)c2)CC1 ; Cc1ccc(N2CCN(CCO)CC2)cc1C(=O)N[C@H](C)c1cccc2ccccc12]

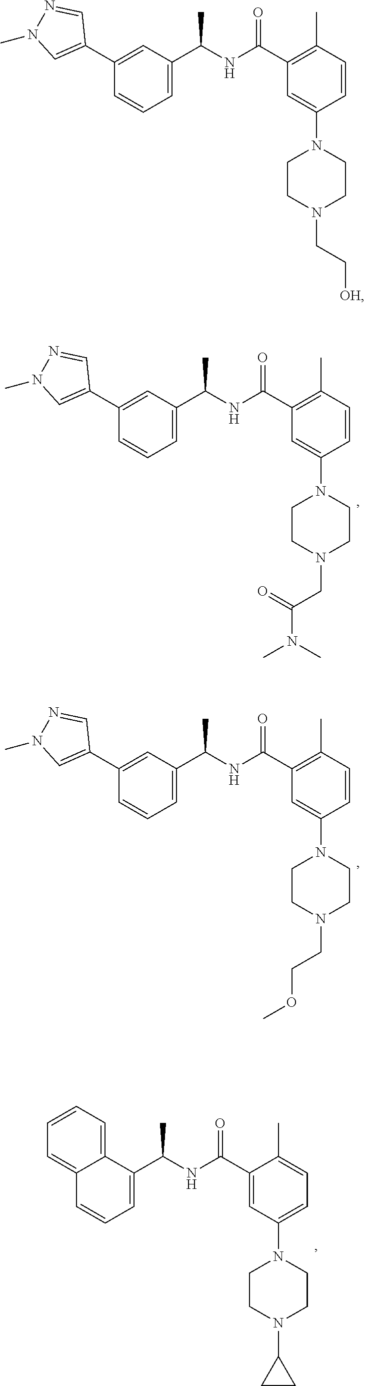 [SMILES: Cc1ccc(N2CCN(CCO)CC2)cc1C(=O)N[C@H](C)c1cccc(-c2cnn(C)c2)c1 ; Cc1ccc(N2CCN(CC(=O)N(C)C)CC2)cc1C(=O)N[C@H](C)c1cccc(-c2cnn(C)c2)c1 ; COCCN1CCN(c2ccc(C)c(C(=O)N[C@H](C)c3cccc(-c4cnn(C)c4)c3)c2)CC1 ; Cc1ccc(N2CCN(C3CC3)CC2)cc1C(=O)N[C@H](C)c1cccc2ccccc12]

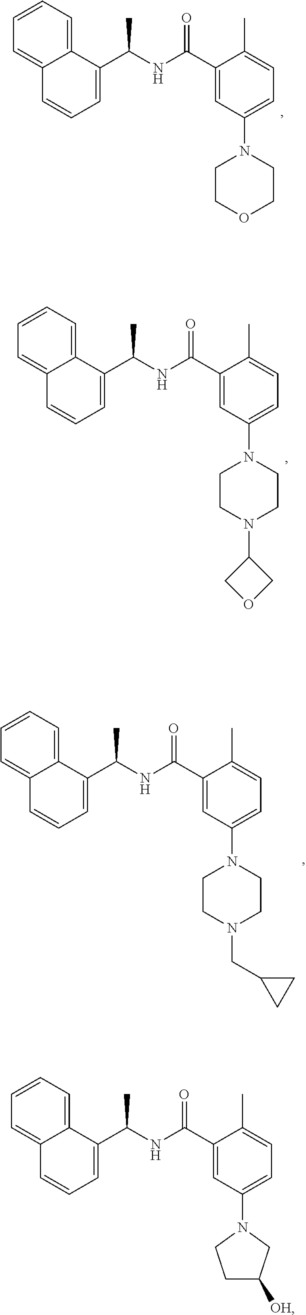 [SMILES: Cc1ccc(N2CCOCC2)cc1C(=O)N[C@H](C)c1cccc2ccccc12 ; Cc1ccc(N2CCN(C3COC3)CC2)cc1C(=O)N[C@H](C)c1cccc2ccccc12 ; Cc1ccc(N2CCN(CC3CC3)CC2)cc1C(=O)N[C@H](C)c1cccc2ccccc12 ; Cc1ccc(N2CC[C@H](O)C2)cc1C(=O)N[C@H](C)c1cccc2ccccc12]

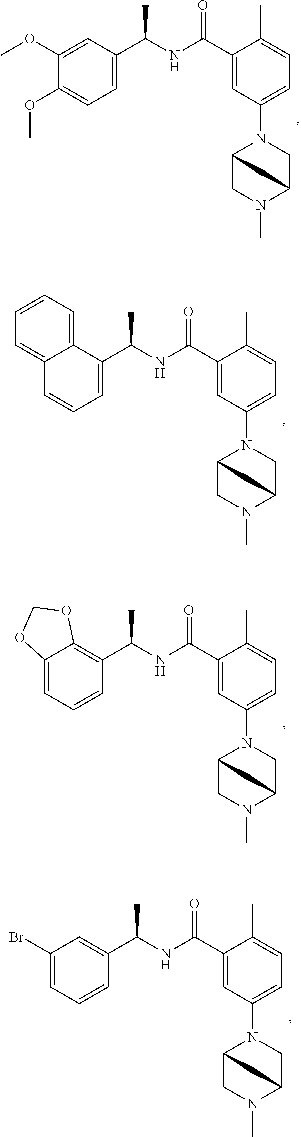 [SMILES: COc1ccc([C@@H](C)NC(=O)c2cc(N3C[C@H]4C[C@@H]3CN4C)ccc2C)cc1OC ; Cc1ccc(N2C[C@H]3C[C@@H]2CN3C)cc1C(=O)N[C@H](C)c1cccc2ccccc12 ; Cc1ccc(N2C[C@H]3C[C@@H]2CN3C)cc1C(=O)N[C@H](C)c1cccc2c1OCO2 ; Cc1ccc(N2C[C@H]3C[C@@H]2CN3C)cc1C(=O)N[C@H](C)c1cccc(Br)c1]

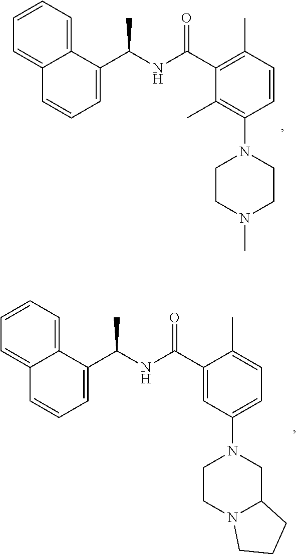 [SMILES: Cc1ccc(N2CCN(C)CC2)c(C)c1C(=O)N[C@H](C)c1cccc2ccccc12 ; Cc1ccc(N2CCN3CCCC3C2)cc1C(=O)N[C@H](C)c1cccc2ccccc12]

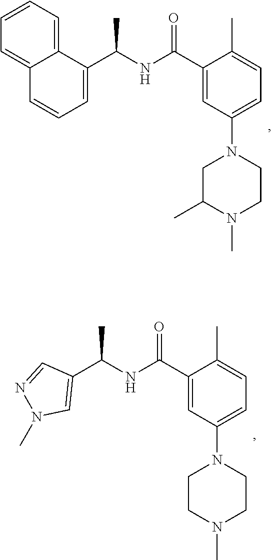 [SMILES: Cc1ccc(N2CCN(C)C(C)C2)cc1C(=O)N[C@H](C)c1cccc2ccccc12 ; Cc1ccc(N2CCN(C)CC2)cc1C(=O)N[C@H](C)c1cnn(C)c1]

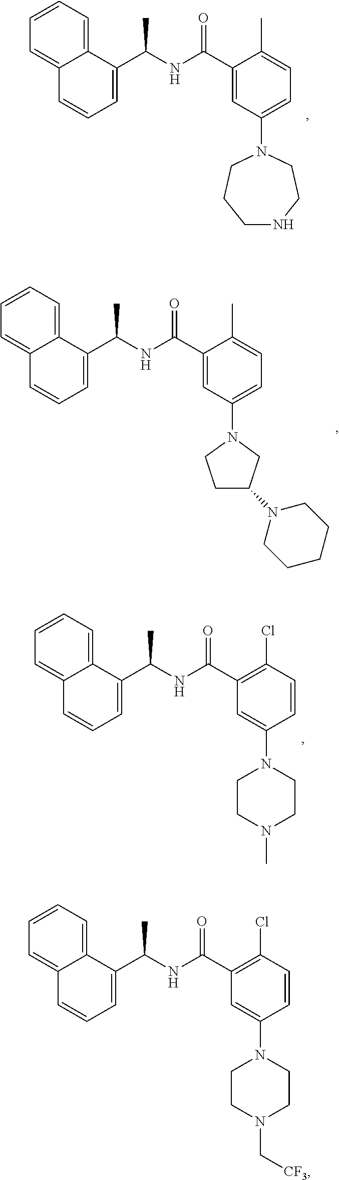 [SMILES: Cc1ccc(N2CCCNCC2)cc1C(=O)N[C@H](C)c1cccc2ccccc12 ; Cc1ccc(N2CC[C@@H](N3CCCCC3)C2)cc1C(=O)N[C@H](C)c1cccc2ccccc12 ; C[C@@H](NC(=O)c1cc(N2CCN(C)CC2)ccc1Cl)c1cccc2ccccc12 ; C[C@@H](NC(=O)c1cc(N2CCN(CC(F)(F)F)CC2)ccc1Cl)c1cccc2ccccc12]

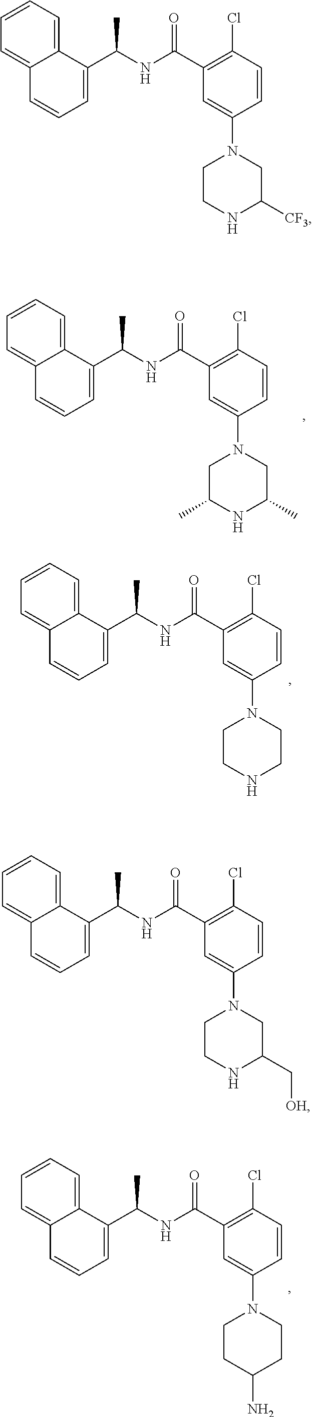 [SMILES: C[C@@H](NC(=O)c1cc(N2CCNC(C(F)(F)F)C2)ccc1Cl)c1cccc2ccccc12 ; C[C@@H]1CN(c2ccc(Cl)c(C(=O)N[C@H](C)c3cccc4ccccc34)c2)C[C@H](C)N1 ; C[C@@H](NC(=O)c1cc(N2CCNCC2)ccc1Cl)c1cccc2ccccc12 ; C[C@@H](NC(=O)c1cc(N2CCNC(CO)C2)ccc1Cl)c1cccc2ccccc12 ; C[C@@H](NC(=O)c1cc(N2CCC(N)CC2)ccc1Cl)c1cccc2ccccc12]

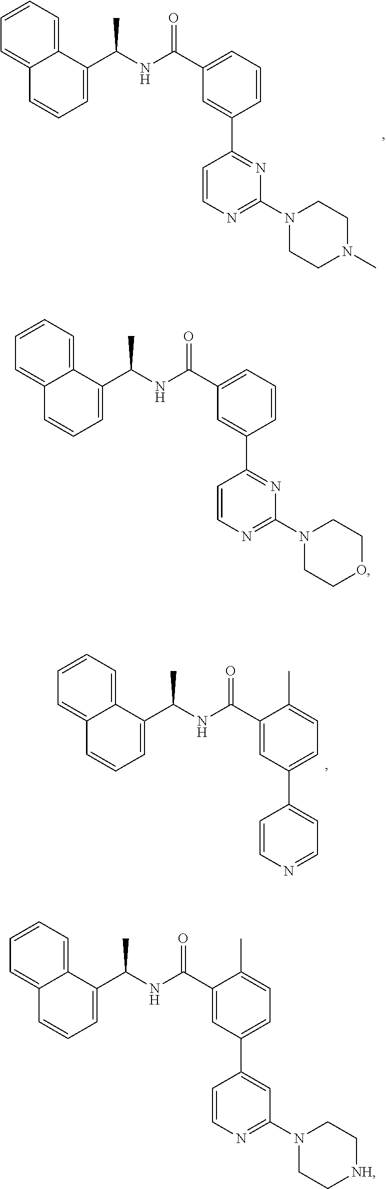 [SMILES: C[C@@H](NC(=O)c1cccc(-c2ccnc(N3CCN(C)CC3)n2)c1)c1cccc2ccccc12 ; C[C@@H](NC(=O)c1cccc(-c2ccnc(N3CCOCC3)n2)c1)c1cccc2ccccc12 ; Cc1ccc(-c2ccncc2)cc1C(=O)N[C@H](C)c1cccc2ccccc12 ; Cc1ccc(-c2ccnc(N3CCNCC3)c2)cc1C(=O)N[C@H](C)c1cccc2ccccc12]

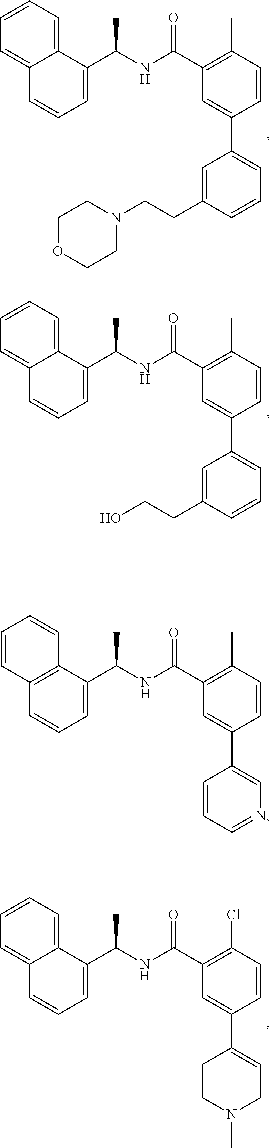 [SMILES: Cc1ccc(-c2cccc(CCN3CCOCC3)c2)cc1C(=O)N[C@H](C)c1cccc2ccccc12 ; Cc1ccc(-c2cccc(CCO)c2)cc1C(=O)N[C@H](C)c1cccc2ccccc12 ; Cc1ccc(-c2cccnc2)cc1C(=O)N[C@H](C)c1cccc2ccccc12 ; C[C@@H](NC(=O)c1cc(C2=CCN(C)CC2)ccc1Cl)c1cccc2ccccc12]

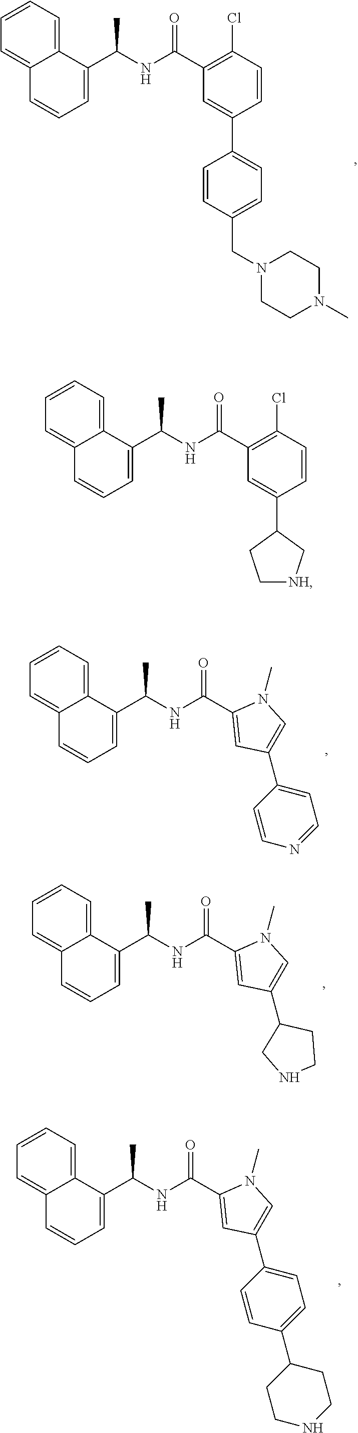 [SMILES: C[C@@H](NC(=O)c1cc(-c2ccc(CN3CCN(C)CC3)cc2)ccc1Cl)c1cccc2ccccc12 ; C[C@@H](NC(=O)c1cc(C2CCNC2)ccc1Cl)c1cccc2ccccc12 ; C[C@@H](NC(=O)c1cc(-c2ccncc2)cn1C)c1cccc2ccccc12 ; C[C@@H](NC(=O)c1cc(C2CCNC2)cn1C)c1cccc2ccccc12 ; C[C@@H](NC(=O)c1cc(-c2ccc(C3CCNCC3)cc2)cn1C)c1cccc2ccccc12]

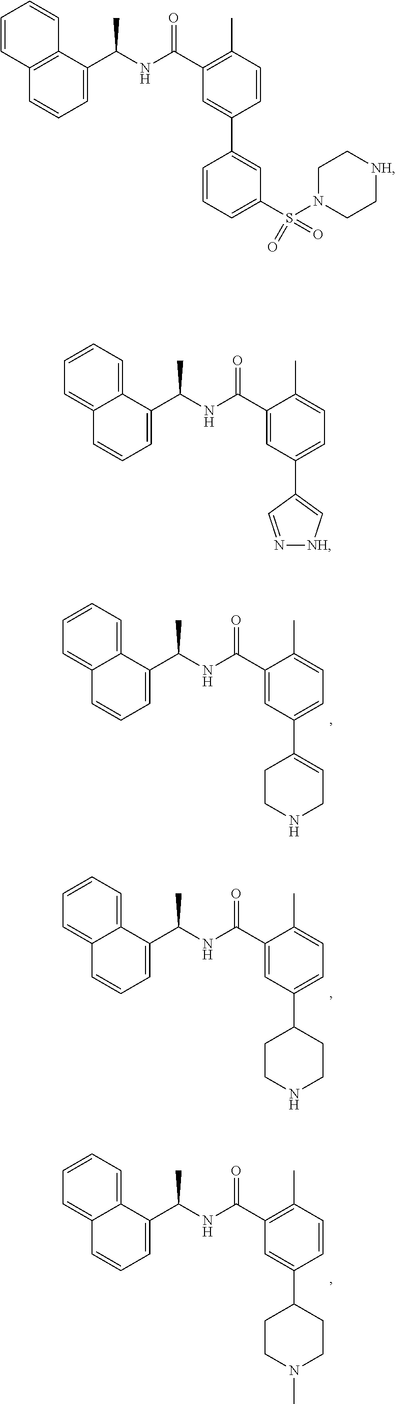 [SMILES: Cc1ccc(-c2cccc(S(=O)(=O)N3CCNCC3)c2)cc1C(=O)N[C@H](C)c1cccc2ccccc12 ; Cc1ccc(-c2cn[nH]c2)cc1C(=O)N[C@H](C)c1cccc2ccccc12 ; Cc1ccc(C2=CCNCC2)cc1C(=O)N[C@H](C)c1cccc2ccccc12 ; Cc1ccc(C2CCNCC2)cc1C(=O)N[C@H](C)c1cccc2ccccc12 ; Cc1ccc(C2CCN(C)CC2)cc1C(=O)N[C@H](C)c1cccc2ccccc12]

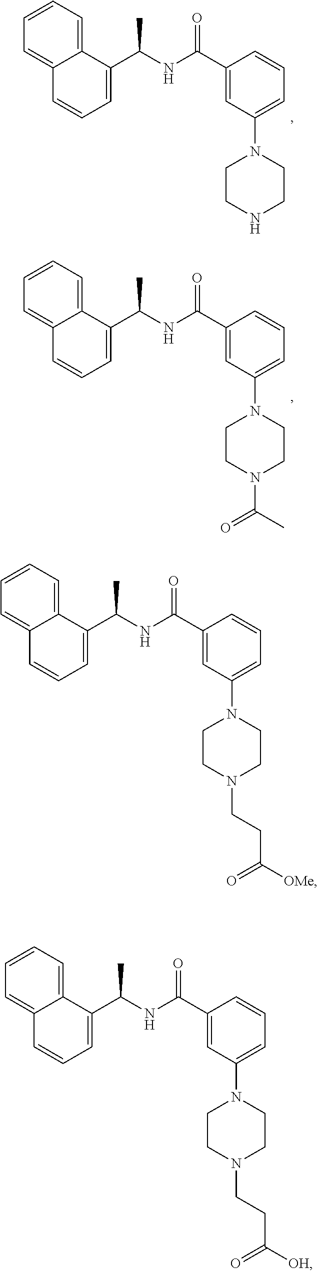 [SMILES: C[C@@H](NC(=O)c1cccc(N2CCNCC2)c1)c1cccc2ccccc12 ; CC(=O)N1CCN(c2cccc(C(=O)N[C@H](C)c3cccc4ccccc34)c2)CC1 ; COC(=O)CCN1CCN(c2cccc(C(=O)N[C@H](C)c3cccc4ccccc34)c2)CC1 ; C[C@@H](NC(=O)c1cccc(N2CCN(CCC(=O)O)CC2)c1)c1cccc2ccccc12]

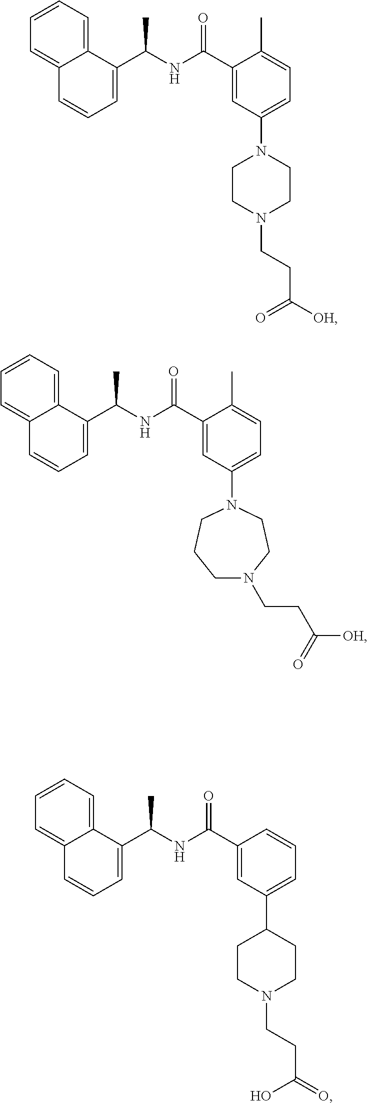 [SMILES: Cc1ccc(N2CCN(CCC(=O)O)CC2)cc1C(=O)N[C@H](C)c1cccc2ccccc12 ; Cc1ccc(N2CCCN(CCC(=O)O)CC2)cc1C(=O)N[C@H](C)c1cccc2ccccc12 ; C[C@@H](NC(=O)c1cccc(C2CCN(CCC(=O)O)CC2)c1)c1cccc2ccccc12]

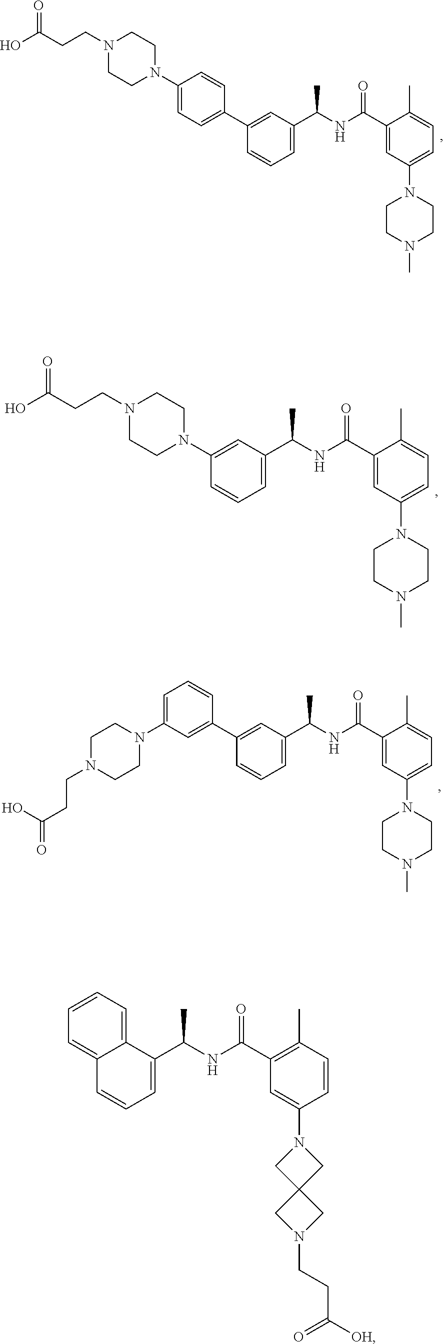 [SMILES: Cc1ccc(N2CCN(C)CC2)cc1C(=O)N[C@H](C)c1cccc(-c2ccc(N3CCN(CCC(=O)O)CC3)cc2)c1 ; Cc1ccc(N2CCN(C)CC2)cc1C(=O)N[C@H](C)c1cccc(N2CCN(CCC(=O)O)CC2)c1 ; Cc1ccc(N2CCN(C)CC2)cc1C(=O)N[C@H](C)c1cccc(-c2cccc(N3CCN(CCC(=O)O)CC3)c2)c1 ; Cc1ccc(N2CC3(CN(CCC(=O)O)C3)C2)cc1C(=O)N[C@H](C)c1cccc2ccccc12]

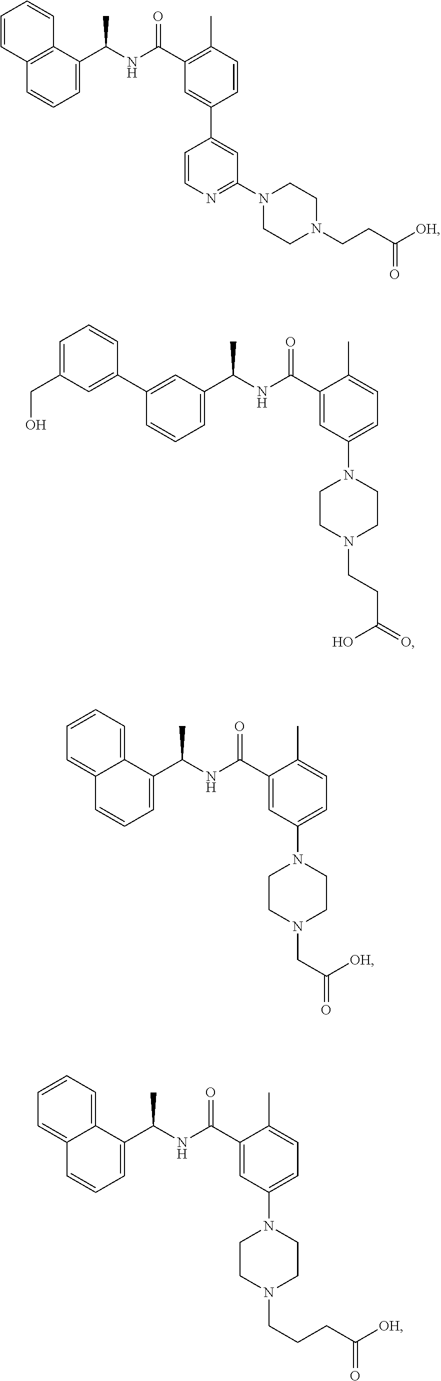 [SMILES: Cc1ccc(-c2ccnc(N3CCN(CCC(=O)O)CC3)c2)cc1C(=O)N[C@H](C)c1cccc2ccccc12 ; Cc1ccc(N2CCN(CCC(=O)O)CC2)cc1C(=O)N[C@H](C)c1cccc(-c2cccc(CO)c2)c1 ; Cc1ccc(N2CCN(CC(=O)O)CC2)cc1C(=O)N[C@H](C)c1cccc2ccccc12 ; Cc1ccc(N2CCN(CCCC(=O)O)CC2)cc1C(=O)N[C@H](C)c1cccc2ccccc12]

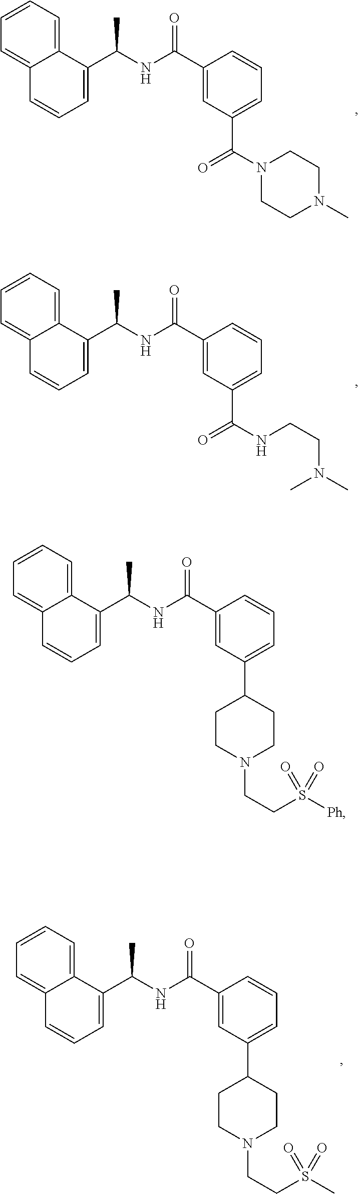 [SMILES: C[C@@H](NC(=O)c1cccc(C(=O)N2CCN(C)CC2)c1)c1cccc2ccccc12 ; C[C@@H](NC(=O)c1cccc(C(=O)NCCN(C)C)c1)c1cccc2ccccc12 ; C[C@@H](NC(=O)c1cccc(C2CCN(CCS(=O)(=O)c3ccccc3)CC2)c1)c1cccc2ccccc12 ; C[C@@H](NC(=O)c1cccc(C2CCN(CCS(C)(=O)=O)CC2)c1)c1cccc2ccccc12]

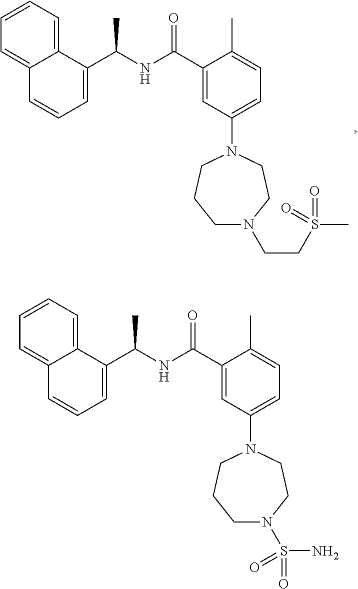 [SMILES: Cc1ccc(N2CCCN(CCS(C)(=O)=O)CC2)cc1C(=O)N[C@H](C)c1cccc2ccccc12 ; Cc1ccc(N2CCCN(S(N)(=O)=O)CC2)cc1C(=O)N[C@H](C)c1cccc2ccccc12]

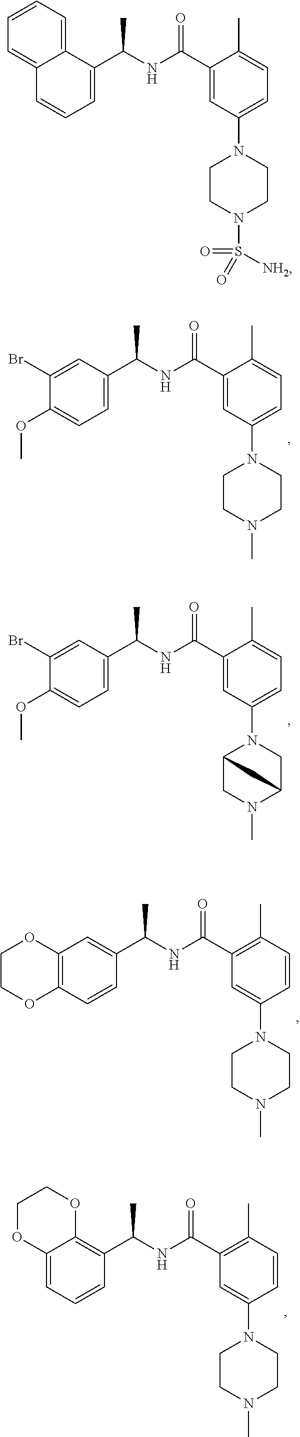 [SMILES: Cc1ccc(N2CCN(S(N)(=O)=O)CC2)cc1C(=O)N[C@H](C)c1cccc2ccccc12 ; COc1ccc([C@@H](C)NC(=O)c2cc(N3CCN(C)CC3)ccc2C)cc1Br ; COc1ccc([C@@H](C)NC(=O)c2cc(N3C[C@H]4C[C@@H]3CN4C)ccc2C)cc1Br ; Cc1ccc(N2CCN(C)CC2)cc1C(=O)N[C@H](C)c1ccc2c(c1)OCCO2 ; Cc1ccc(N2CCN(C)CC2)cc1C(=O)N[C@H](C)c1cccc2c1OCCO2]

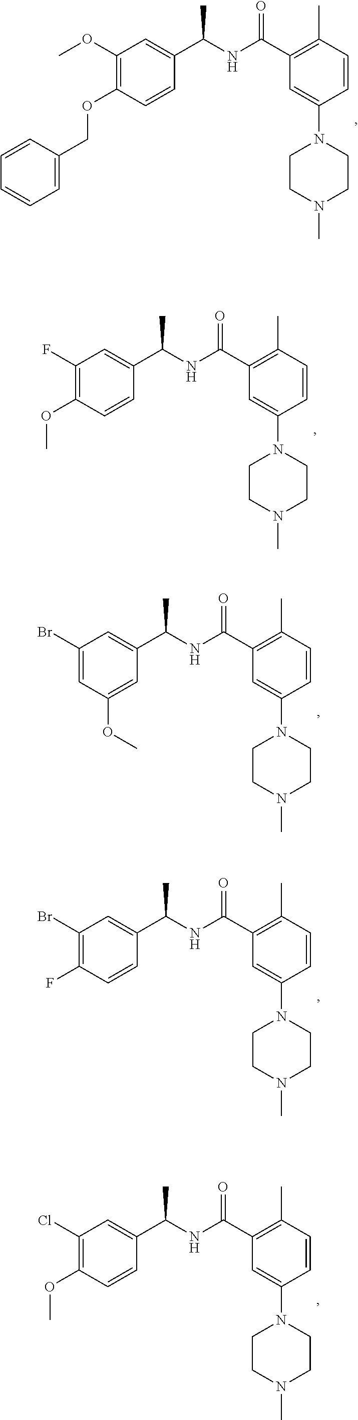 [SMILES: COc1cc([C@@H](C)NC(=O)c2cc(N3CCN(C)CC3)ccc2C)ccc1OCc1ccccc1 ; COc1ccc([C@@H](C)NC(=O)c2cc(N3CCN(C)CC3)ccc2C)cc1F ; COc1cc(Br)cc([C@@H](C)NC(=O)c2cc(N3CCN(C)CC3)ccc2C)c1 ; Cc1ccc(N2CCN(C)CC2)cc1C(=O)N[C@H](C)c1ccc(F)c(Br)c1 ; COc1ccc([C@@H](C)NC(=O)c2cc(N3CCN(C)CC3)ccc2C)cc1Cl]

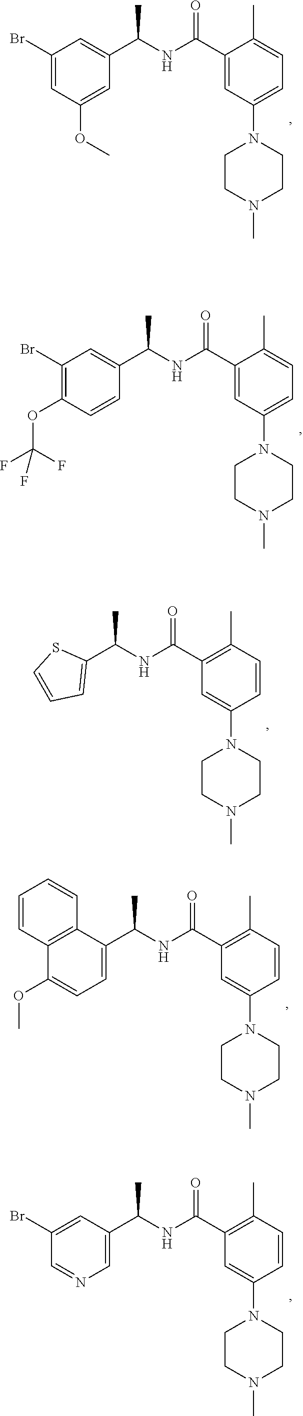 [SMILES: COc1cc(Br)cc([C@@H](C)NC(=O)c2cc(N3CCN(C)CC3)ccc2C)c1 ; Cc1ccc(N2CCN(C)CC2)cc1C(=O)N[C@H](C)c1ccc(OC(F)(F)F)c(Br)c1 ; Cc1ccc(N2CCN(C)CC2)cc1C(=O)N[C@H](C)c1cccs1 ; COc1ccc([C@@H](C)NC(=O)c2cc(N3CCN(C)CC3)ccc2C)c2ccccc12 ; Cc1ccc(N2CCN(C)CC2)cc1C(=O)N[C@H](C)c1cncc(Br)c1]

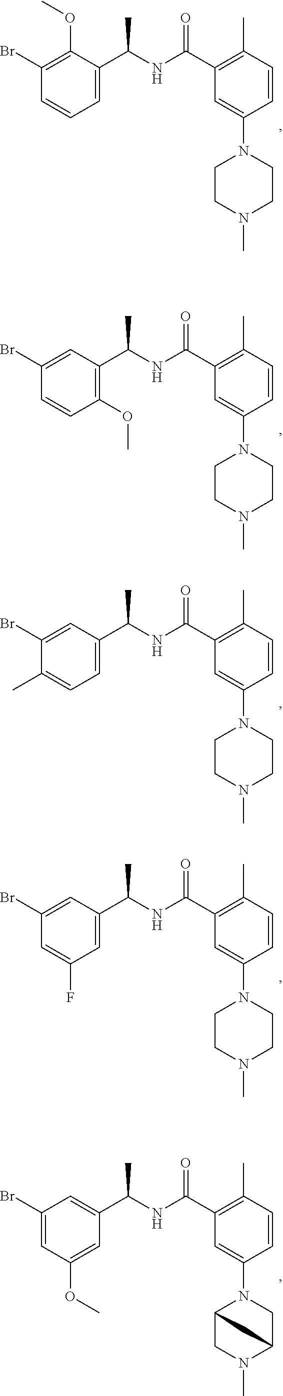

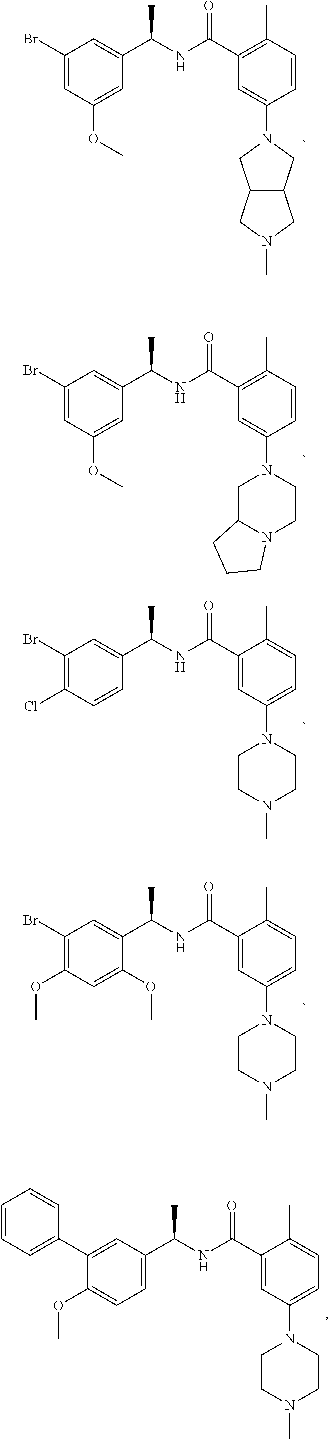

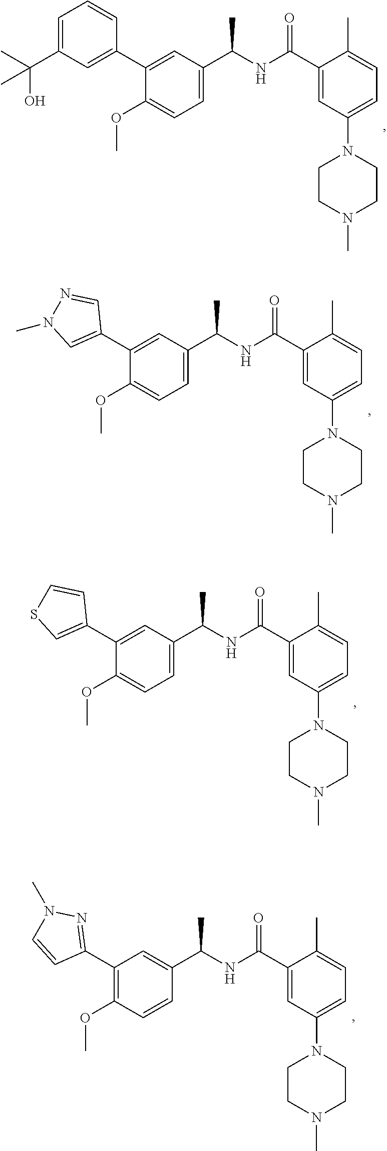 [SMILES: COc1ccc([C@@H](C)NC(=O)c2cc(N3CCN(C)CC3)ccc2C)cc1-c1cccc(C(C)(C)O)c1 ; COc1ccc([C@@H](C)NC(=O)c2cc(N3CCN(C)CC3)ccc2C)cc1-c1cnn(C)c1 ; COc1ccc([C@@H](C)NC(=O)c2cc(N3CCN(C)CC3)ccc2C)cc1-c1ccsc1 ; COc1ccc([C@@H](C)NC(=O)c2cc(N3CCN(C)CC3)ccc2C)cc1-c1ccn(C)n1]

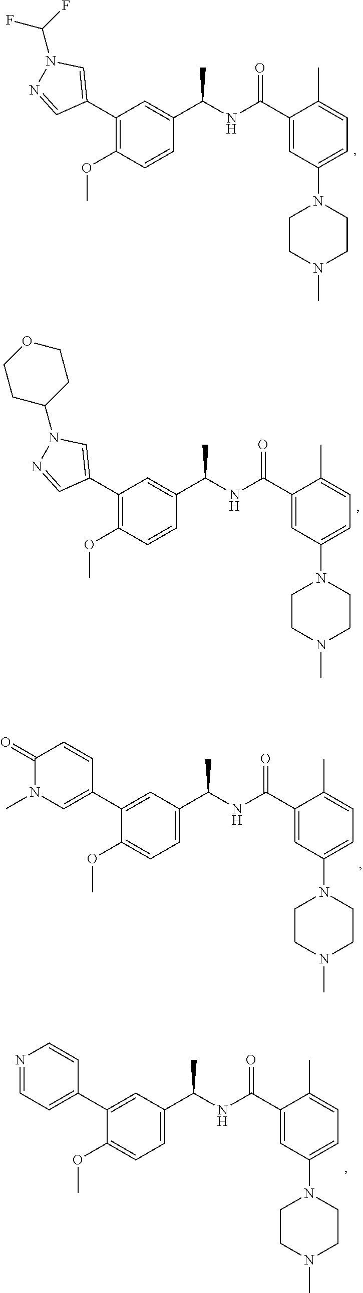 [SMILES: COc1ccc([C@@H](C)NC(=O)c2cc(N3CCN(C)CC3)ccc2C)cc1-c1cnn(C(F)F)c1 ; COc1ccc([C@@H](C)NC(=O)c2cc(N3CCN(C)CC3)ccc2C)cc1-c1cnn(C2CCOCC2)c1 ; COc1ccc([C@@H](C)NC(=O)c2cc(N3CCN(C)CC3)ccc2C)cc1-c1ccc(=O)n(C)c1 ; COc1ccc([C@@H](C)NC(=O)c2cc(N3CCN(C)CC3)ccc2C)cc1-c1ccncc1]

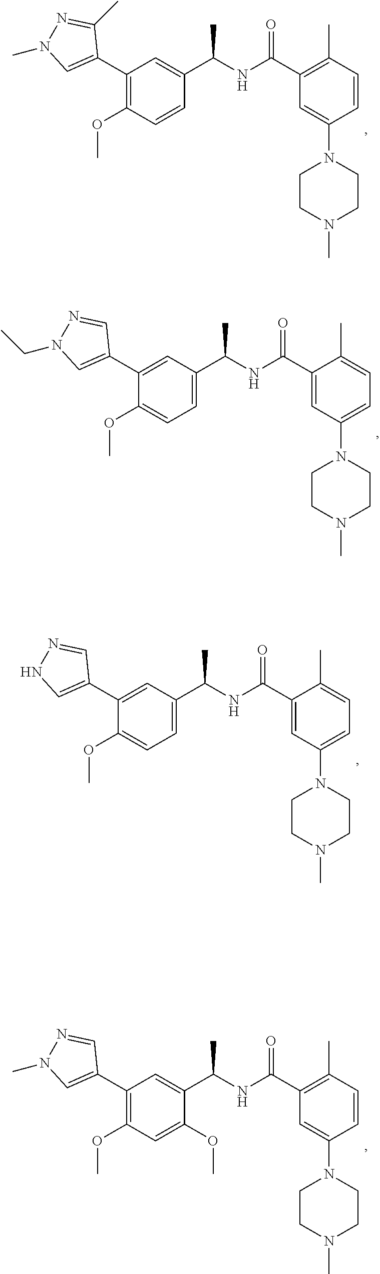 [SMILES: COc1ccc([C@@H](C)NC(=O)c2cc(N3CCN(C)CC3)ccc2C)cc1-c1cn(C)nc1C ; CCn1cc(-c2cc([C@@H](C)NC(=O)c3cc(N4CCN(C)CC4)ccc3C)ccc2OC)cn1 ; COc1ccc([C@@H](C)NC(=O)c2cc(N3CCN(C)CC3)ccc2C)cc1-c1cn[nH]c1 ; COc1cc(OC)c([C@@H](C)NC(=O)c2cc(N3CCN(C)CC3)ccc2C)cc1-c1cnn(C)c1]

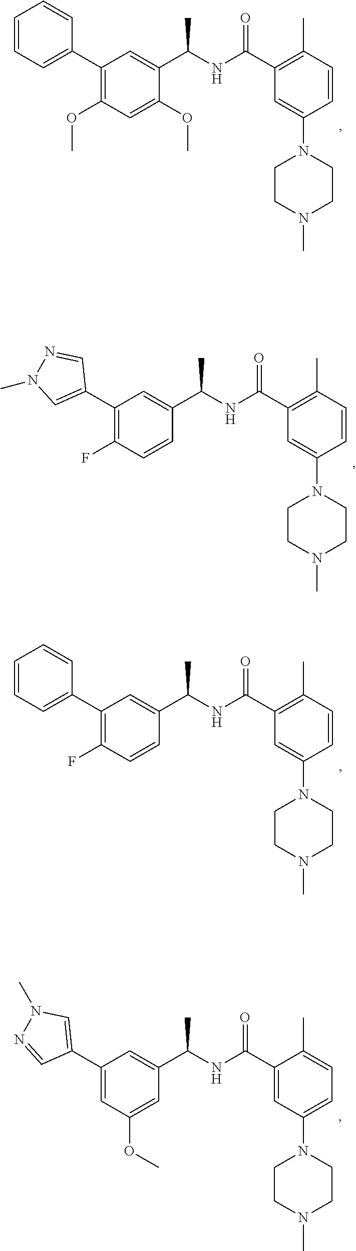 [SMILES: COc1cc(OC)c([C@@H](C)NC(=O)c2cc(N3CCN(C)CC3)ccc2C)cc1-c1ccccc1 ; Cc1ccc(N2CCN(C)CC2)cc1C(=O)N[C@H](C)c1ccc(F)c(-c2cnn(C)c2)c1 ; Cc1ccc(N2CCN(C)CC2)cc1C(=O)N[C@H](C)c1ccc(F)c(-c2ccccc2)c1 ; COc1cc(-c2cnn(C)c2)cc([C@@H](C)NC(=O)c2cc(N3CCN(C)CC3)ccc2C)c1]

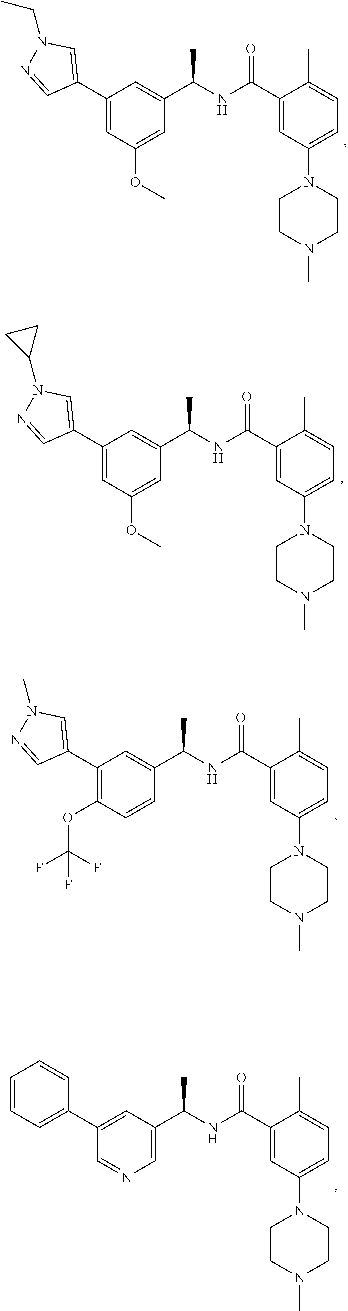 [SMILES: CCn1cc(-c2cc(OC)cc([C@@H](C)NC(=O)c3cc(N4CCN(C)CC4)ccc3C)c2)cn1 ; COc1cc(-c2cnn(C3CC3)c2)cc([C@@H](C)NC(=O)c2cc(N3CCN(C)CC3)ccc2C)c1 ; Cc1ccc(N2CCN(C)CC2)cc1C(=O)N[C@H](C)c1ccc(OC(F)(F)F)c(-c2cnn(C)c2)c1 ; Cc1ccc(N2CCN(C)CC2)cc1C(=O)N[C@H](C)c1cncc(-c2ccccc2)c1]

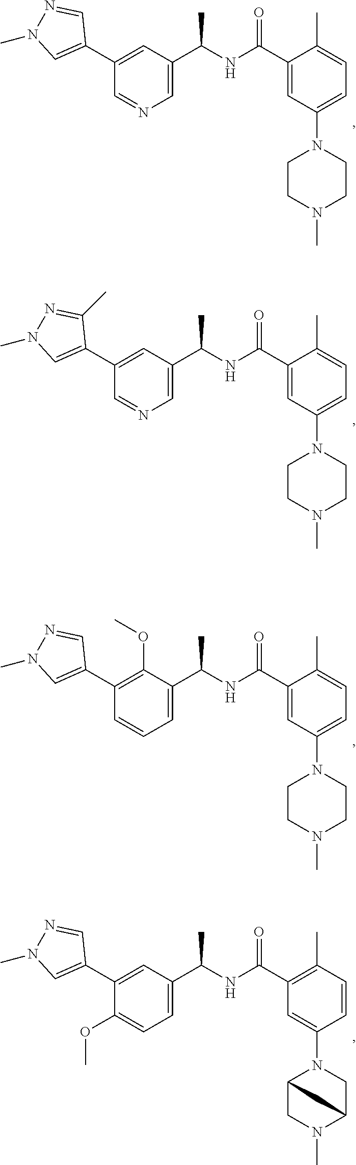 [SMILES: Cc1ccc(N2CCN(C)CC2)cc1C(=O)N[C@H](C)c1cncc(-c2cnn(C)c2)c1 ; Cc1ccc(N2CCN(C)CC2)cc1C(=O)N[C@H](C)c1cncc(-c2cn(C)nc2C)c1 ; COc1c(-c2cnn(C)c2)cccc1[C@@H](C)NC(=O)c1cc(N2CCN(C)CC2)ccc1C ; COc1ccc([C@@H](C)NC(=O)c2cc(N3C[C@H]4C[C@@H]3CN4C)ccc2C)cc1-c1cnn(C)c1]

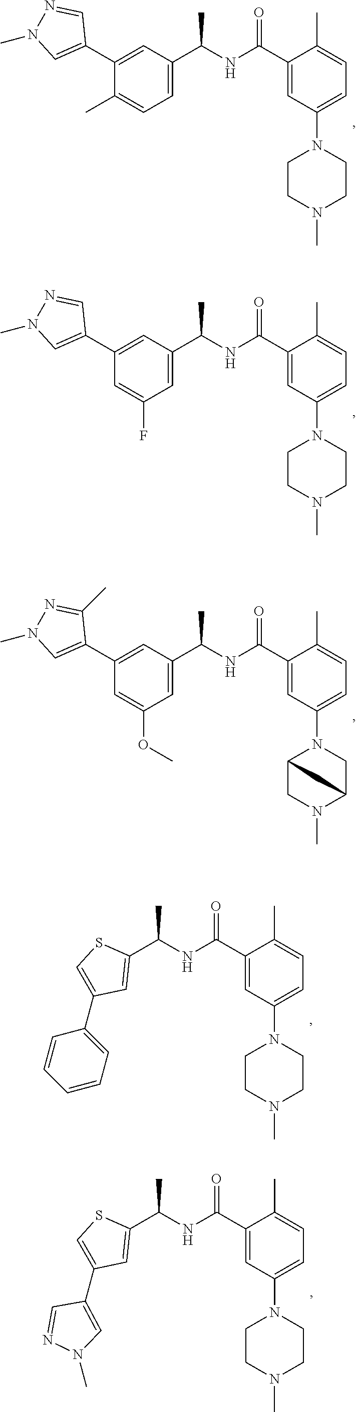

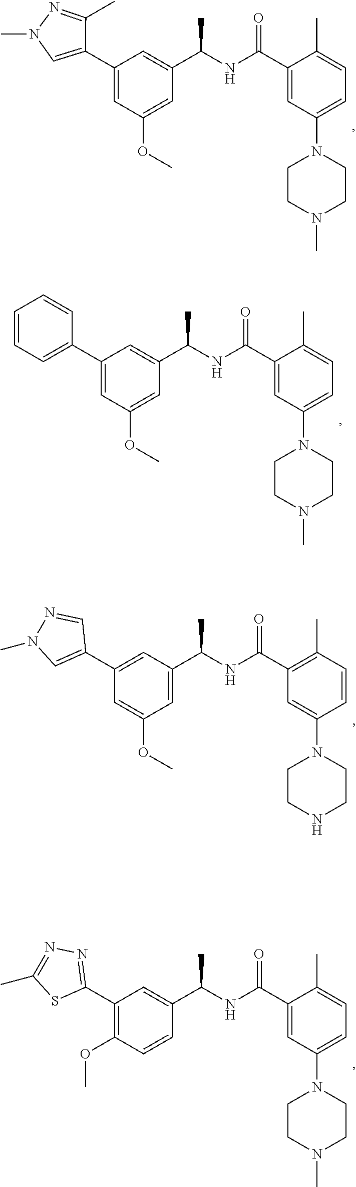 [SMILES: COc1cc(-c2cn(C)nc2C)cc([C@@H](C)NC(=O)c2cc(N3CCN(C)CC3)ccc2C)c1 ; COc1cc(-c2ccccc2)cc([C@@H](C)NC(=O)c2cc(N3CCN(C)CC3)ccc2C)c1 ; COc1cc(-c2cnn(C)c2)cc([C@@H](C)NC(=O)c2cc(N3CCNCC3)ccc2C)c1 ; COc1ccc([C@@H](C)NC(=O)c2cc(N3CCN(C)CC3)ccc2C)cc1-c1nnc(C)s1]

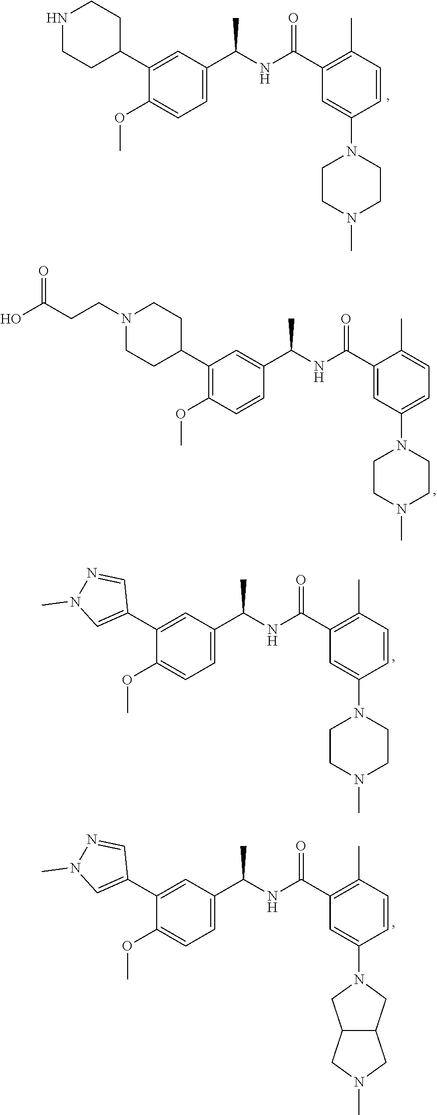

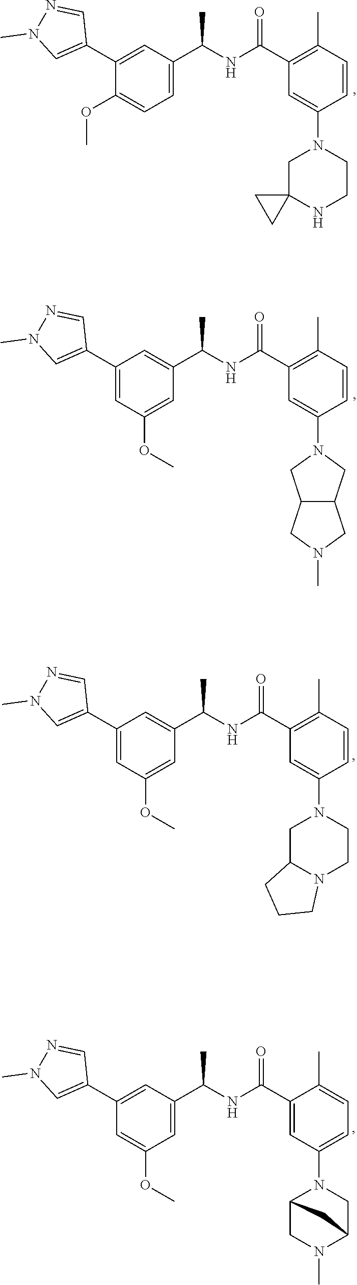 [SMILES: COc1ccc([C@@H](C)NC(=O)c2cc(N3CCNC4(CC4)C3)ccc2C)cc1-c1cnn(C)c1 ; COc1cc(-c2cnn(C)c2)cc([C@@H](C)NC(=O)c2cc(N3CC4CN(C)CC4C3)ccc2C)c1 ; COc1cc(-c2cnn(C)c2)cc([C@@H](C)NC(=O)c2cc(N3CCN4CCCC4C3)ccc2C)c1 ; COc1cc(-c2cnn(C)c2)cc([C@@H](C)NC(=O)c2cc(N3C[C@H]4C[C@@H]3CN4C)ccc2C)c1]

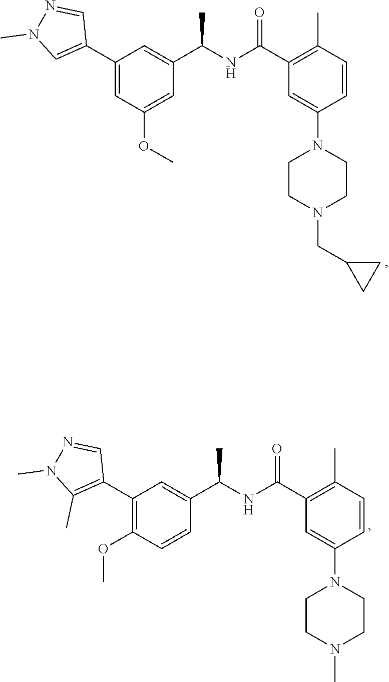 [SMILES: COc1cc(-c2cnn(C)c2)cc([C@@H](C)NC(=O)c2cc(N3CCN(CC4CC4)CC3)ccc2C)c1 ; COc1ccc([C@@H](C)NC(=O)c2cc(N3CCN(C)CC3)ccc2C)cc1-c1cnn(C)c1C]

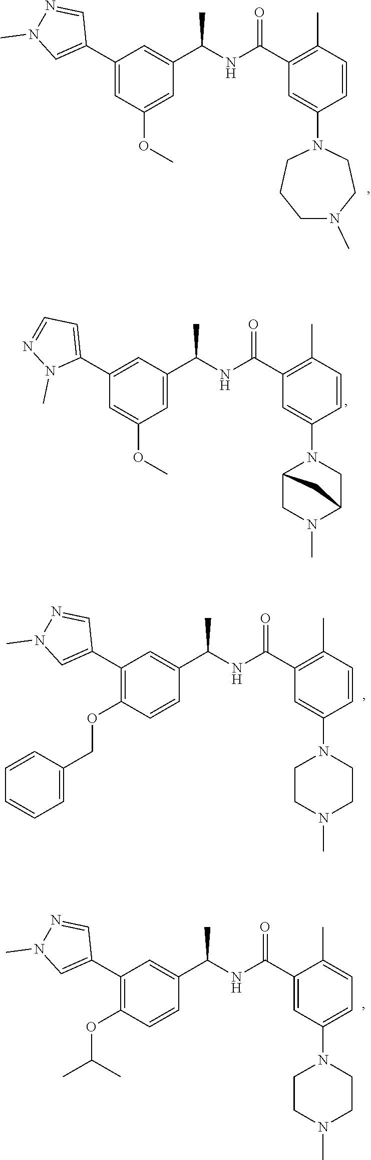 [SMILES: COc1cc(-c2cnn(C)c2)cc([C@@H](C)NC(=O)c2cc(N3CCCN(C)CC3)ccc2C)c1 ; COc1cc(-c2ccnn2C)cc([C@@H](C)NC(=O)c2cc(N3C[C@H]4C[C@@H]3CN4C)ccc2C)c1 ; Cc1ccc(N2CCN(C)CC2)cc1C(=O)N[C@H](C)c1ccc(OCc2ccccc2)c(-c2cnn(C)c2)c1 ; Cc1ccc(N2CCN(C)CC2)cc1C(=O)N[C@H](C)c1ccc(OC(C)C)c(-c2cnn(C)c2)c1]

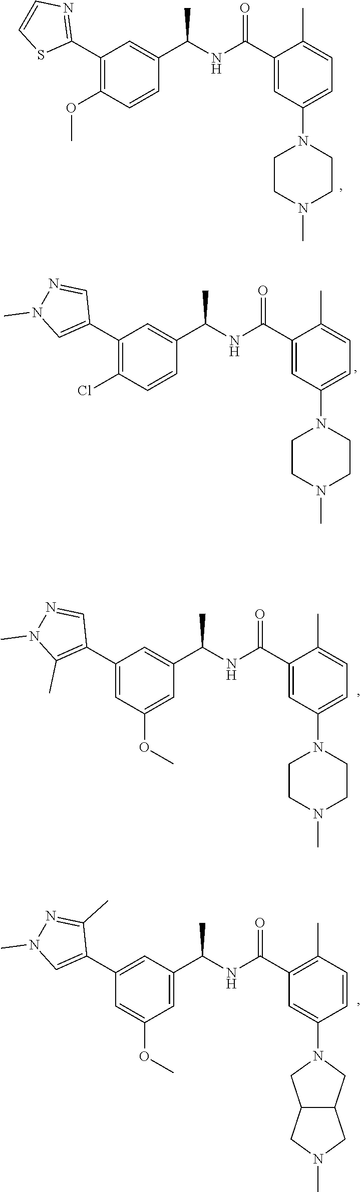 [SMILES: COc1ccc([C@@H](C)NC(=O)c2cc(N3CCN(C)CC3)ccc2C)cc1-c1nccs1 ; Cc1ccc(N2CCN(C)CC2)cc1C(=O)N[C@H](C)c1ccc(Cl)c(-c2cnn(C)c2)c1 ; COc1cc(-c2cnn(C)c2C)cc([C@@H](C)NC(=O)c2cc(N3CCN(C)CC3)ccc2C)c1 ; COc1cc(-c2cn(C)nc2C)cc([C@@H](C)NC(=O)c2cc(N3CC4CN(C)CC4C3)ccc2C)c1]

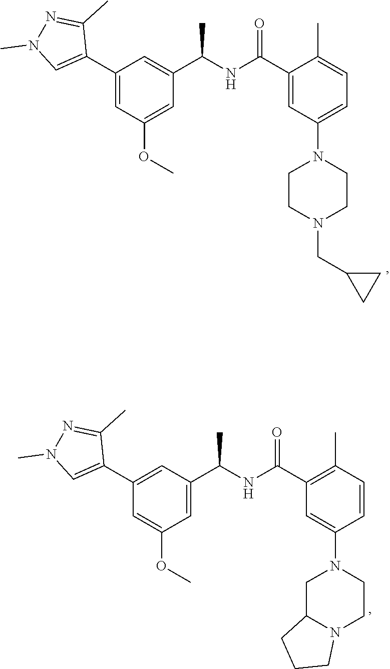 [SMILES: COc1cc(-c2cn(C)nc2C)cc([C@@H](C)NC(=O)c2cc(N3CCN(CC4CC4)CC3)ccc2C)c1 ; COc1cc(-c2cn(C)nc2C)cc([C@@H](C)NC(=O)c2cc(N3CCN4CCCC4C3)ccc2C)c1]

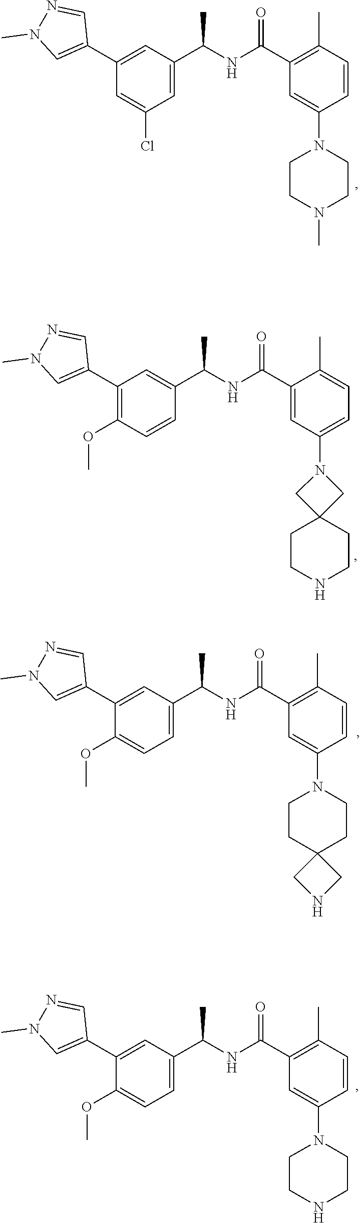 [SMILES: Cc1ccc(N2CCN(C)CC2)cc1C(=O)N[C@H](C)c1cc(Cl)cc(-c2cnn(C)c2)c1 ; COc1ccc([C@@H](C)NC(=O)c2cc(N3CC4(CCNCC4)C3)ccc2C)cc1-c1cnn(C)c1 ; COc1ccc([C@@H](C)NC(=O)c2cc(N3CCC4(CC3)CNC4)ccc2C)cc1-c1cnn(C)c1 ; COc1ccc([C@@H](C)NC(=O)c2cc(N3CCNCC3)ccc2C)cc1-c1cnn(C)c1]

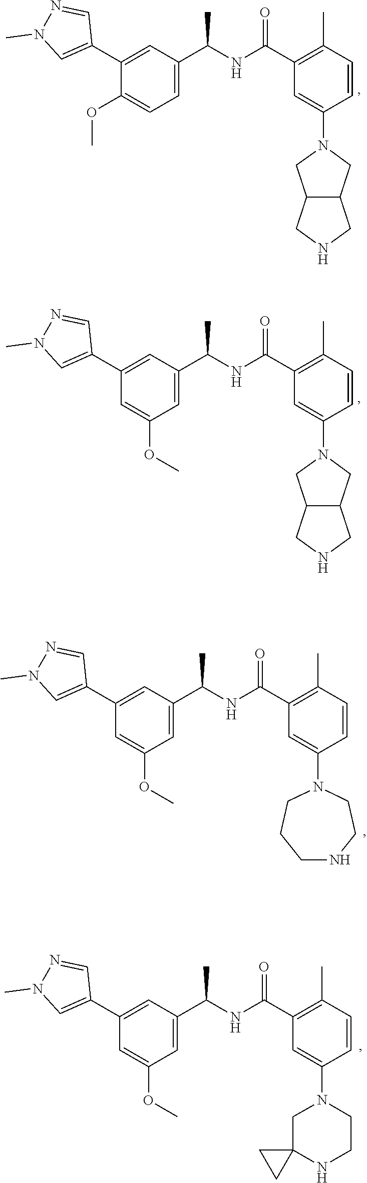 [SMILES: COc1ccc([C@@H](C)NC(=O)c2cc(N3CC4CNCC4C3)ccc2C)cc1-c1cnn(C)c1 ; COc1cc(-c2cnn(C)c2)cc([C@@H](C)NC(=O)c2cc(N3CC4CNCC4C3)ccc2C)c1 ; COc1cc(-c2cnn(C)c2)cc([C@@H](C)NC(=O)c2cc(N3CCCNCC3)ccc2C)c1 ; COc1cc(-c2cnn(C)c2)cc([C@@H](C)NC(=O)c2cc(N3CCNC4(CC4)C3)ccc2C)c1]

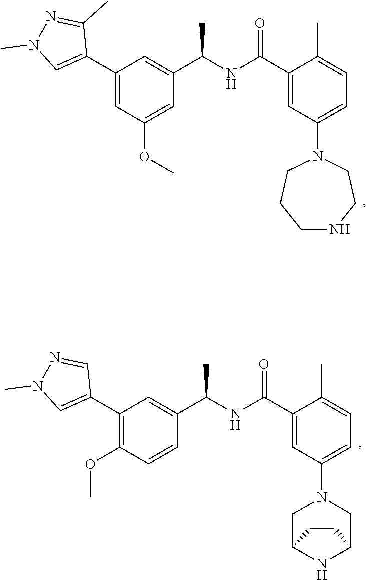 [SMILES: COc1cc(-c2cn(C)nc2C)cc([C@@H](C)NC(=O)c2cc(N3CCCNCC3)ccc2C)c1 ; COc1ccc([C@@H](C)NC(=O)c2cc(N3C[C@H]4CC[C@@H](C3)N4)ccc2C)cc1-c1cnn(C)c1]

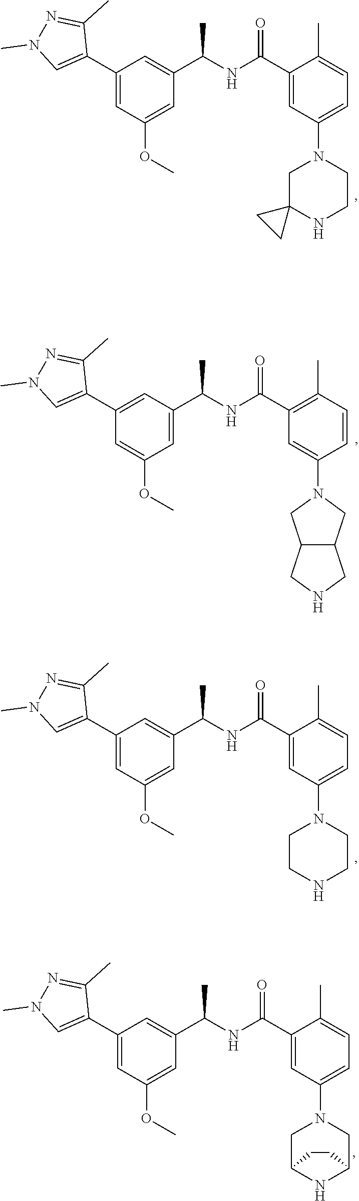 [SMILES: COc1cc(-c2cn(C)nc2C)cc([C@@H](C)NC(=O)c2cc(N3CCNC4(CC4)C3)ccc2C)c1 ; COc1cc(-c2cn(C)nc2C)cc([C@@H](C)NC(=O)c2cc(N3CC4CNCC4C3)ccc2C)c1 ; COc1cc(-c2cn(C)nc2C)cc([C@@H](C)NC(=O)c2cc(N3CCNCC3)ccc2C)c1 ; COc1cc(-c2cn(C)nc2C)cc([C@@H](C)NC(=O)c2cc(N3C[C@H]4CC[C@@H](C3)N4)ccc2C)c1]

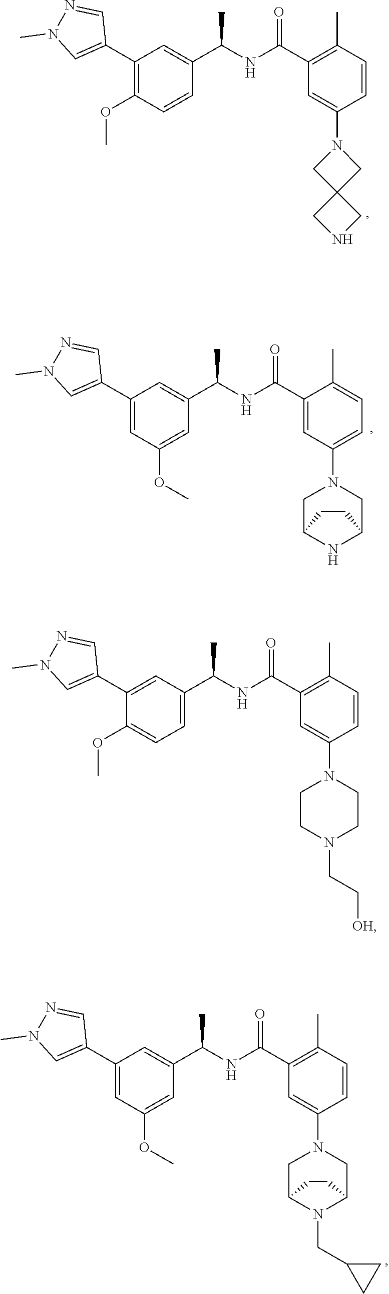 [SMILES: COc1ccc([C@@H](C)NC(=O)c2cc(N3CC4(CNC4)C3)ccc2C)cc1-c1cnn(C)c1 ; COc1cc(-c2cnn(C)c2)cc([C@@H](C)NC(=O)c2cc(N3C[C@H]4CC[C@@H](C3)N4)ccc2C)c1 ; COc1ccc([C@@H](C)NC(=O)c2cc(N3CCN(CCO)CC3)ccc2C)cc1-c1cnn(C)c1 ; COc1cc(-c2cnn(C)c2)cc([C@@H](C)NC(=O)c2cc(N3C[C@H]4CC[C@@H](C3)N4CC3CC3)ccc2C)c1]

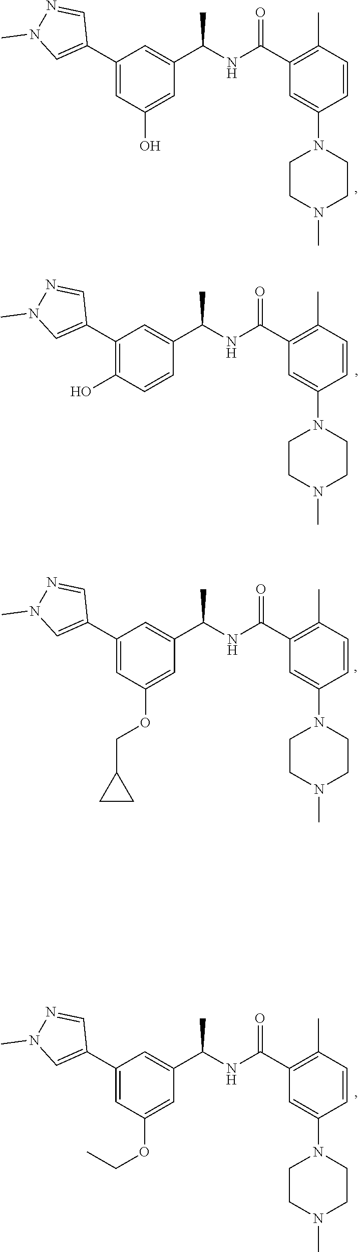

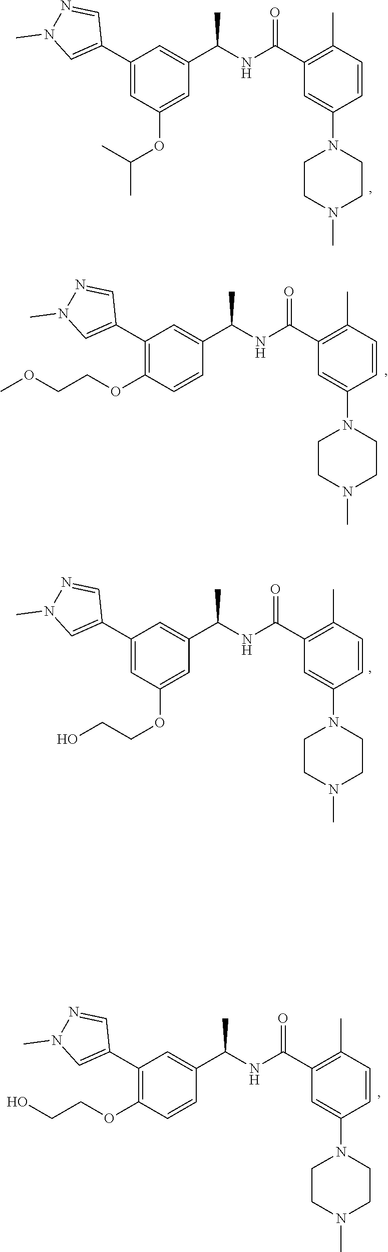 [SMILES: Cc1ccc(N2CCN(C)CC2)cc1C(=O)N[C@H](C)c1cc(OC(C)C)cc(-c2cnn(C)c2)c1 ; COCCOc1ccc([C@@H](C)NC(=O)c2cc(N3CCN(C)CC3)ccc2C)cc1-c1cnn(C)c1 ; Cc1ccc(N2CCN(C)CC2)cc1C(=O)N[C@H](C)c1cc(OCCO)cc(-c2cnn(C)c2)c1 ; Cc1ccc(N2CCN(C)CC2)cc1C(=O)N[C@H](C)c1ccc(OCCO)c(-c2cnn(C)c2)c1]

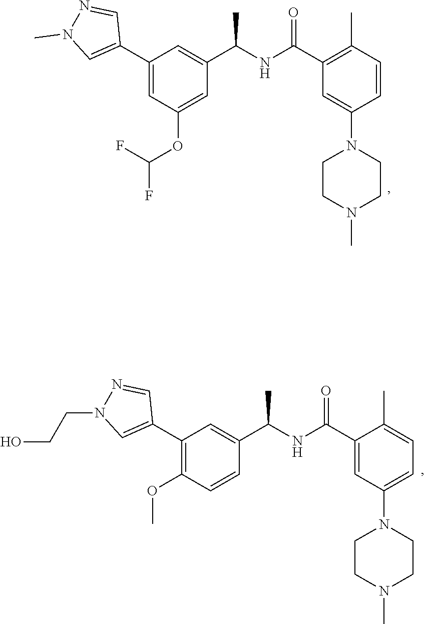 [SMILES: Cc1ccc(N2CCN(C)CC2)cc1C(=O)N[C@H](C)c1cc(OC(F)F)cc(-c2cnn(C)c2)c1 ; COc1ccc([C@@H](C)NC(=O)c2cc(N3CCN(C)CC3)ccc2C)cc1-c1cnn(CCO)c1]

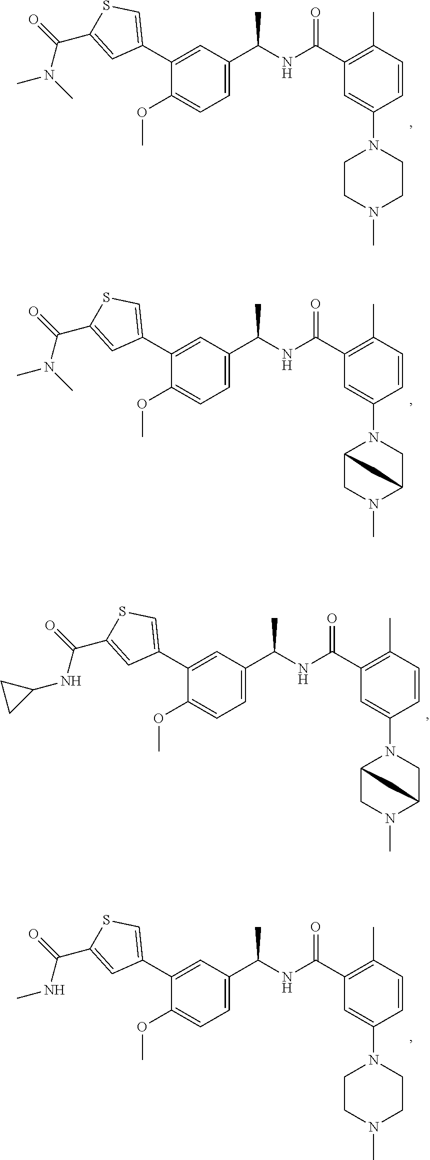 [SMILES: COc1ccc([C@@H](C)NC(=O)c2cc(N3CCN(C)CC3)ccc2C)cc1-c1csc(C(=O)N(C)C)c1 ; COc1ccc([C@@H](C)NC(=O)c2cc(N3C[C@H]4C[C@@H]3CN4C)ccc2C)cc1-c1csc(C(=O)N(C)C)c1 ; COc1ccc([C@@H](C)NC(=O)c2cc(N3C[C@H]4C[C@@H]3CN4C)ccc2C)cc1-c1csc(C(=O)NC2CC2)c1 ; CNC(=O)c1cc(-c2cc([C@@H](C)NC(=O)c3cc(N4CCN(C)CC4)ccc3C)ccc2OC)cs1]

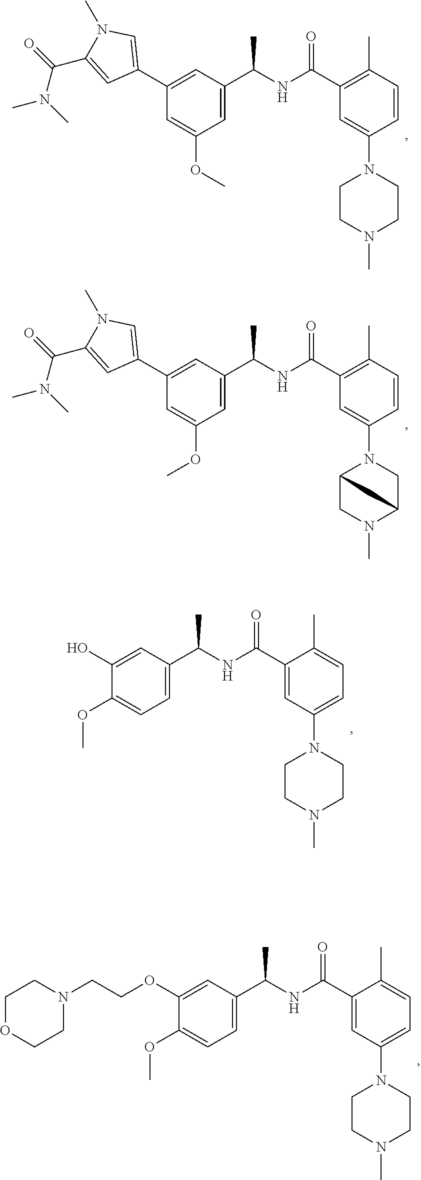 [SMILES: COc1cc(-c2cc(C(=O)N(C)C)n(C)c2)cc([C@@H](C)NC(=O)c2cc(N3CCN(C)CC3)ccc2C)c1 ; COc1cc(-c2cc(C(=O)N(C)C)n(C)c2)cc([C@@H](C)NC(=O)c2cc(N3C[C@H]4C[C@@H]3CN4C)ccc2C)c1 ; COc1ccc([C@@H](C)NC(=O)c2cc(N3CCN(C)CC3)ccc2C)cc1O ; COc1ccc([C@@H](C)NC(=O)c2cc(N3CCN(C)CC3)ccc2C)cc1OCCN1CCOCC1]

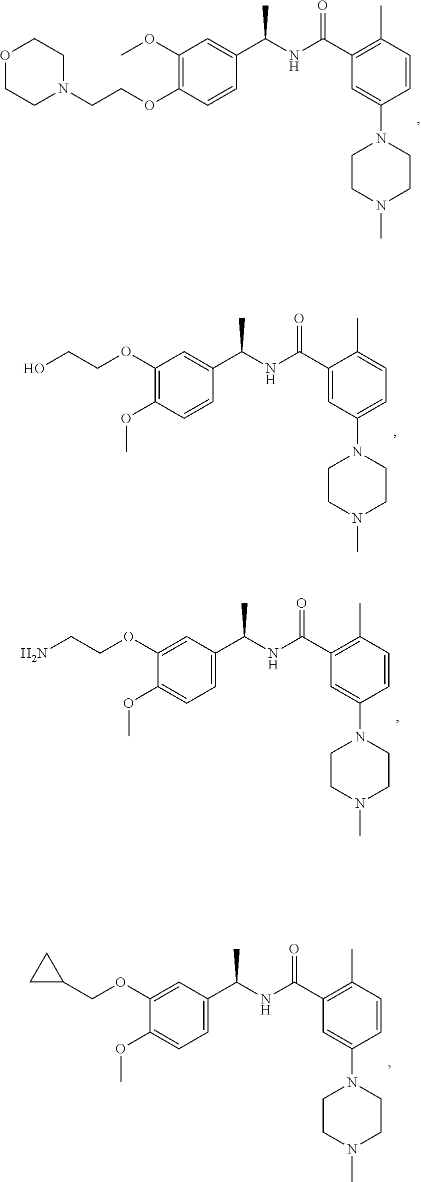 [SMILES: COc1cc([C@@H](C)NC(=O)c2cc(N3CCN(C)CC3)ccc2C)ccc1OCCN1CCOCC1 ; COc1ccc([C@@H](C)NC(=O)c2cc(N3CCN(C)CC3)ccc2C)cc1OCCO ; COc1ccc([C@@H](C)NC(=O)c2cc(N3CCN(C)CC3)ccc2C)cc1OCCN ; COc1ccc([C@@H](C)NC(=O)c2cc(N3CCN(C)CC3)ccc2C)cc1OCC1CC1]

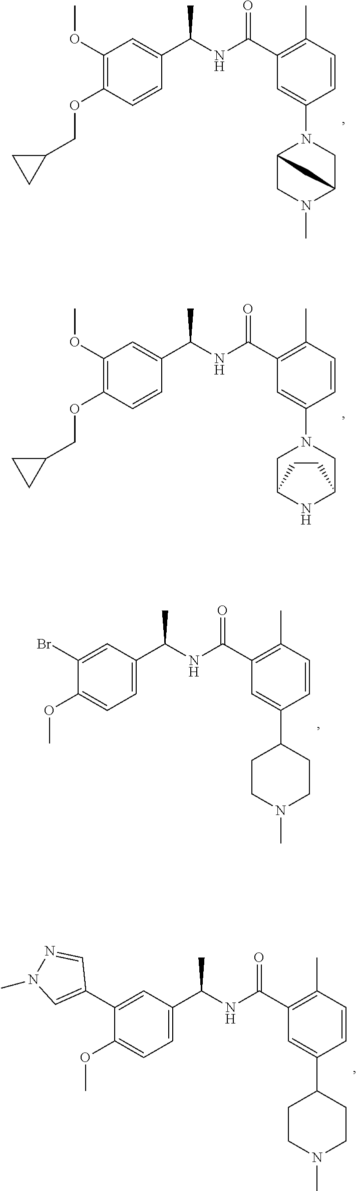 [SMILES: COc1cc([C@@H](C)NC(=O)c2cc(N3C[C@H]4C[C@@H]3CN4C)ccc2C)ccc1OCC1CC1 ; COc1cc([C@@H](C)NC(=O)c2cc(N3C[C@H]4CC[C@@H](C3)N4)ccc2C)ccc1OCC1CC1 ; COc1ccc([C@@H](C)NC(=O)c2cc(C3CCN(C)CC3)ccc2C)cc1Br ; COc1ccc([C@@H](C)NC(=O)c2cc(C3CCN(C)CC3)ccc2C)cc1-c1cnn(C)c1]

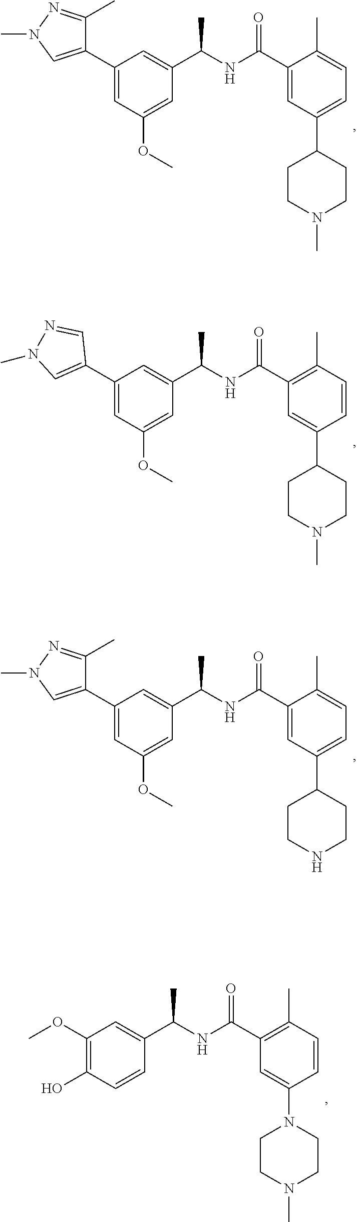 [SMILES: COc1cc(-c2cn(C)nc2C)cc([C@@H](C)NC(=O)c2cc(C3CCN(C)CC3)ccc2C)c1 ; COc1cc(-c2cnn(C)c2)cc([C@@H](C)NC(=O)c2cc(C3CCN(C)CC3)ccc2C)c1 ; COc1cc(-c2cn(C)nc2C)cc([C@@H](C)NC(=O)c2cc(C3CCNCC3)ccc2C)c1 ; COc1cc([C@@H](C)NC(=O)c2cc(N3CCN(C)CC3)ccc2C)ccc1O]

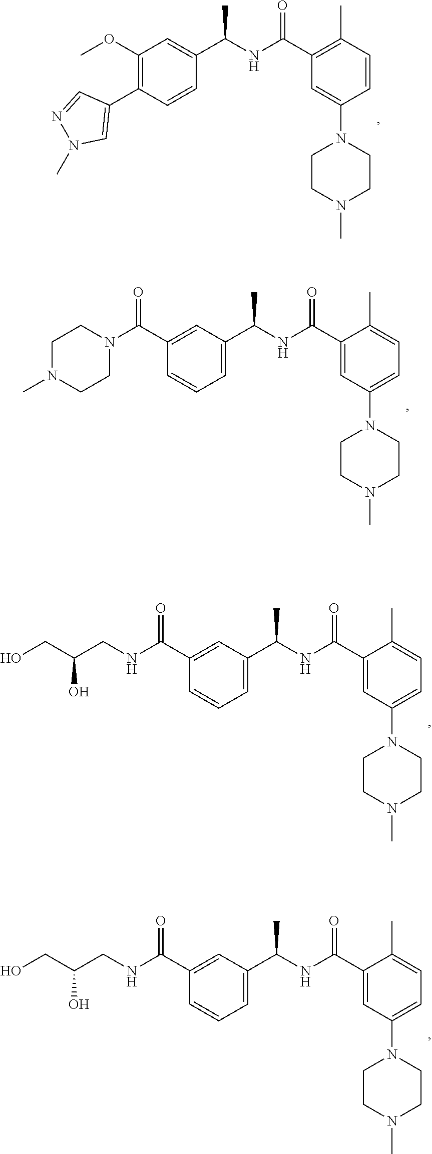 [SMILES: COc1cc([C@@H](C)NC(=O)c2cc(N3CCN(C)CC3)ccc2C)ccc1-c1cnn(C)c1 ; Cc1ccc(N2CCN(C)CC2)cc1C(=O)N[C@H](C)c1cccc(C(=O)N2CCN(C)CC2)c1 ; Cc1ccc(N2CCN(C)CC2)cc1C(=O)N[C@H](C)c1cccc(C(=O)NC[C@@H](O)CO)c1 ; Cc1ccc(N2CCN(C)CC2)cc1C(=O)N[C@H](C)c1cccc(C(=O)NC[C@H](O)CO)c1]

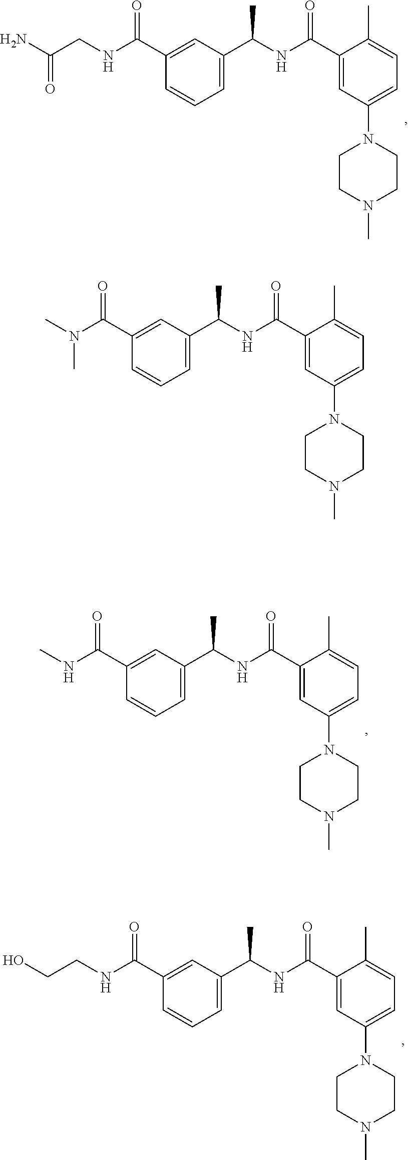 [SMILES: Cc1ccc(N2CCN(C)CC2)cc1C(=O)N[C@H](C)c1cccc(C(=O)NCC(N)=O)c1 ; Cc1ccc(N2CCN(C)CC2)cc1C(=O)N[C@H](C)c1cccc(C(=O)N(C)C)c1 ; CNC(=O)c1cccc([C@@H](C)NC(=O)c2cc(N3CCN(C)CC3)ccc2C)c1 ; Cc1ccc(N2CCN(C)CC2)cc1C(=O)N[C@H](C)c1cccc(C(=O)NCCO)c1]

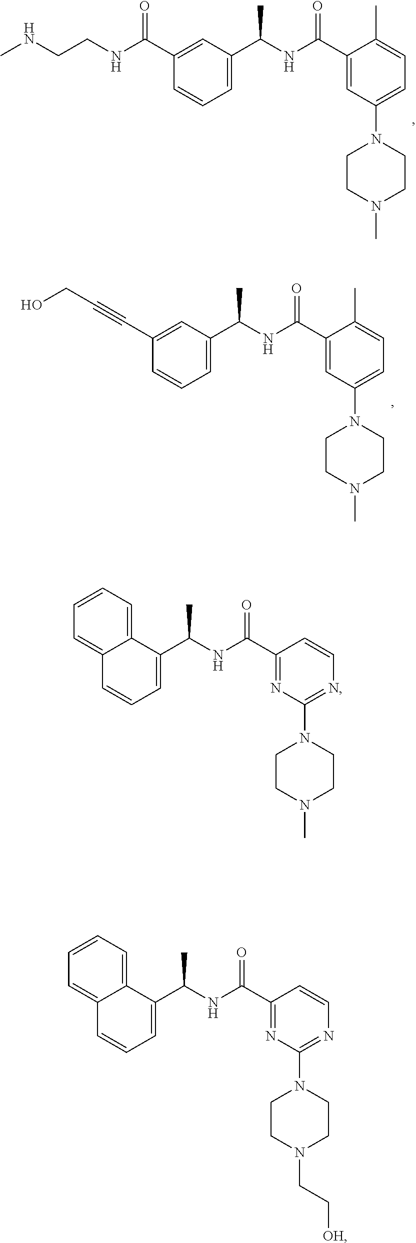 [SMILES: CNCCNC(=O)c1cccc([C@@H](C)NC(=O)c2cc(N3CCN(C)CC3)ccc2C)c1 ; Cc1ccc(N2CCN(C)CC2)cc1C(=O)N[C@H](C)c1cccc(C#CCO)c1 ; C[C@@H](NC(=O)c1ccnc(N2CCN(C)CC2)n1)c1cccc2ccccc12 ; C[C@@H](NC(=O)c1ccnc(N2CCN(CCO)CC2)n1)c1cccc2ccccc12]

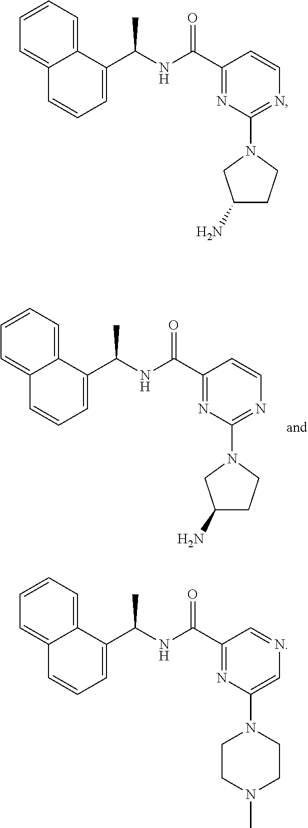 [SMILES: C[C@@H](NC(=O)c1ccnc(N2CC[C@H](N)C2)n1)c1cccc2ccccc12 ; C[C@@H](NC(=O)c1ccnc(N2CC[C@@H](N)C2)n1)c1cccc2ccccc12 ; C[C@@H](NC(=O)c1cncc(N2CCN(C)CC2)n1)c1cccc2ccccc12]

In embodiments, the compound of formula (I) may not be:

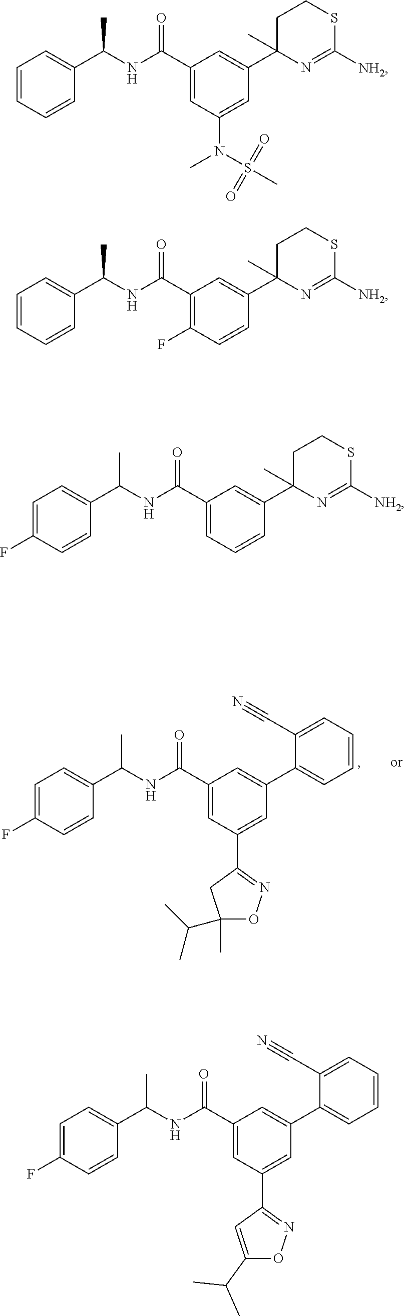 [SMILES: C[C@@H](NC(=O)c1cc(N(C)S(C)(=O)=O)cc(C2(C)CCSC(N)=N2)c1)c1ccccc1 ; C[C@@H](NC(=O)c1cc(C2(C)CCSC(N)=N2)ccc1F)c1ccccc1 ; CC(NC(=O)c1cccc(C2(C)CCSC(N)=N2)c1)c1ccc(F)cc1 ; CC(NC(=O)c1cc(C2=NOC(C)(C(C)C)C2)cc(-c2ccccc2C#N)c1)c1ccc(F)cc1 ; CC(C)c1cc(-c2cc(C(=O)NC(C)c3ccc(F)cc3)cc(-c3ccccc3C#N)c2)no1]

### DETAILED DESCRIPTION

The chemical terms used in the specification have their generally accepted meanings in the art.

The term Cₘ-Cₙ refers to a group with m to n carbon atoms.

The term “halo” refers to fluoro, chloro, bromo and iodo.

The term “alkyl” refers to a linear or branched saturated monovalent hydrocarbon chain. For example, C₁-C₆-alkyl may refer to methyl, ethyl, n-propyl, iso-propyl, n-butyl, sec-butyl, tert-butyl, n-pentyl and n-hexyl. The alkyl groups may be unsubstituted or substituted by one or more substituents. Specific substituents for each alkyl group independently may be fluorine, OR^{a} or NHR^{a}.

The term “alkylene” refers to a linear saturated divalent hydrocarbon chain. The alkylene groups may be unsubstituted or substituted by one or more substituents. Specific substituents for each alkylene group independently may be C₁-C₄-alkyl, fluorine, OR^{a} or NHR^{a}.

The term “haloalkyl” refers to a hydrocarbon group substituted with at least one halogen atom independently chosen at each occurrence from: fluorine, chlorine, bromine and iodine. The halogen atom may be present at any position on the hydrocarbon chain. For example, C₁-C₆-haloalkyl may refer to chloromethyl, fluoromethyl, trifluoromethyl, chloroethyl e.g. 1-chloroethyl and 2-chloroethyl, trichloroethyl e.g. 1,2,2-trichloroethyl, 2,2,2-trichloroethyl, fluoroethyl e.g. 1-fluoroethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl. A haloalkyl group may be a fluoroalkyl group, i.e. a hydrocarbon chain substituted with at least one fluorine atom. Thus, a haloalkyl group may have any amount of halogen substituents. The group may contain a single halogen substituent, it may have two or three halogen substituents, or it may be saturated with halogen substituents.

The term “alkenyl” refers to a branched or linear hydrocarbon group containing at least one double bond. The double bond(s) may be present as the E or Z isomer. The double bond may be at any possible position of the hydrocarbon chain; for example, “C₂-C₆-alkenyl” may refer to ethenyl, propenyl, butenyl, butadienyl, pentenyl, pentadienyl, hexenyl and hexadienyl. The alkenyl groups may be unsubstituted or substituted by one or more substituents. Specific substituents for any saturated carbon atom in each alkenyl group independently may be fluorine, OR^{a} or NHR^{a}.

The term “alkynyl” refers to a branched or linear hydrocarbon chain containing at least one triple bond. The triple bond may be at any possible position of the hydrocarbon chain. For example, “C₂-C₆-alkynyl” may refer to ethynyl, propynyl, butynyl, pentynyl and hexynyl. The alkynyl groups may be unsubstituted or substituted by one or more substituents. Specific substituents for any saturated carbon atom in each alkynyl group independently may be fluorine, OR^{a} or NHR^{a}.

The term “cycloalkyl” refers to a saturated hydrocarbon ring system containing, for example, 3, 4, 5 or 6 carbon atoms. For example, “C₃-C₆-cycloalkyl” may refer to cyclopropyl, cyclobutyl, cyclopentyl, cyclohexyl. The cycloalkyl groups may be unsubstituted or substituted by one or more substituents. Specific substituents for each cycloalkyl group independently may be fluorine, OR^{a} or NHR^{a}.

The term “heterocycloalkyl” may refer to a monocyclic or bicyclic saturated or partially saturated group having the indicated number of atoms in the ring system and comprising 1 or 2 heteroatoms independently selected from O, S and N in the ring system (in other words 1 or 2 of the atoms forming the ring system are selected from O, S and N). By saturated (or fully saturated) it is meant that the ring does not comprise any double bonds. By partially saturated it is meant that the ring may comprise one or two double bonds. This applies particularly to monocyclic rings with from 5 to 6 members. The double bond will typically be between two carbon atoms but may be between a carbon atom and a nitrogen atom. Where a heterocyclalkyl group is bicyclic, it may be a fused bicycle (i.e. the two rings share two adjacent carbon or nitrogen atoms), a spiro-fused bicycle (i.e. the two rings share a single carbon atom) or a bridged bicycle (i.e. the two rings share two non-adjacent carbon or nitrogen atoms). Examples of heterocycloalkyl groups include; piperidine, piperazine, morpholine, thiomorpholine, pyrrolidine, tetrahydrofuran, tetrahydrothiophene, dihydrofuran, tetrahydropyran, dihydropyran, dioxane, azepine. A heterocycloalkyl group may be unsubstituted or substituted by one or more substituents. Specific substituents for any saturated carbon atom in each heterocycloalkyl group may independently be fluorine, OR^{a} or NHR^{a}.

Aryl groups may be any aromatic carbocyclic ring system (i.e. a ring system containing 2(2n+1)π electrons). Aryl groups may have from 6 to 12 carbon atoms in the ring system. Aryl groups will typically be phenyl groups. Aryl groups may be naphthyl groups or biphenyl groups.

In any of the above aspects and embodiments, heteroaryl groups may be any aromatic (i.e. a ring system containing 2(2n+1)π electrons) 5-10 membered ring system comprising from 1 to 4 heteroatoms independently selected from O, S and N (in other words from 1 to 4 of the atoms forming the ring system are selected from 0, S and N). Thus, any heteroaryl groups may be independently selected from: 5 membered heteroaryl groups in which the heteroaromatic ring is substituted with 14 heteroatoms independently selected from O, S and N; and 6-membered heteroaryl groups in which the heteroaromatic ring is substituted with 1-3 (e.g. 1-2) nitrogen atoms; 9-membered bicyclic heteroaryl groups in which the heteroaromatic system is substituted with 1-4 heteroatoms independently selected from O, S and N; 10-membered bicyclic heteroaryl groups in which the heteroaromatic system is substituted with 1-4 nitrogen atoms. Specifically, heteroaryl groups may be independently selected from: pyrrole, furan, thiophene, pyrazole, imidazole, oxazole, isoxazole, triazole, oxadiazole, thiadiazole, tetrazole, pyridine, pyridazine, pyrimidine, pyrazine, triazine, indole, isoindole, benzofuran, isobenzofuran, benzothiophene, indazole, benzimidazole, benzoxazole, benzothiazole, benzisoxazole, purine, quinoline, isoquinoline, cinnoline, quinazoline, quinoxaline, pteridine, phthalazine, naphthyridine.

It may be that, in any group which is an aryl or heteroaryl group, that aryl or heteroaryl group is unsubstituted or is optionally substituted, where chemically possible, by 1 to 5 substituents which are each independently selected at each occurrence from: halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}, SR^{a}, S(O)R^{a}, S(O)₂OR^{a}, S(O)₂R^{a}, S(O)₂NR^{a}R^{a}. CO₂R^{a}C(O)R^{a}, CONR^{a}R^{a}, CR^{b}R^{b}NR^{a}R^{a}, CR^{b}R^{b}OR^{a}, C₁-C₄-alkyl, C₂-C₄-alkenyl, C₂-C₄-alkynyl and C₁-C₄-haloalkyl; wherein R^{a} and R^{b} are as described above for formula I.

Compounds of the invention containing one or more asymmetric carbon atoms can exist as two or more stereoisomers. Mere a compound of the invention contains a double bond such as a C═C or C═N group, geometric cis/trans (or Z/E) isomers are possible. Where structural isomers are interconvertible via a low energy barrier, tautomeric isomerism (‘tautomerism’) can occur. This can take the form of proton tautomerism in compounds of the invention containing, for example, an imino, keto, or oxime group, or so-called valence tautomerism in compounds which contain an aromatic moiety. It follows that a single compound may exhibit more than one type of isomerism.

Included within the scope of the present invention are all stereoisomers, geometric isomers and tautomeric forms of the compounds of the invention, including compounds exhibiting more than one type of isomerism, and mixtures of one or more thereof.

The compounds of the invention may be obtained, stored and/or used in the form of a pharmaceutically acceptable salt. Suitable salts include, but are not limited to, salts of acceptable inorganic acids such as hydrochloric, sulfuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulfonic, toluenesulfonic, benzenesulfonic, salicylic, sulfanilic, aspartic, glutamic, edetic, stearic, palmitic, oleic, lauric, pantothenic, tannic, ascorbic and valeric acids. Suitable salts also include salts of inorganic and organic bases, e.g. counterions such as Na, Ca, K, Li, Mg, ammonium, trimethylsulfonium. The compounds may also be obtained, stored and/or used in the form of an N-oxide. Also included are acid addition salts or base salts wherein the counter ion is optically active; for example, d-lactate or 1-lysine, or racemic; for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers when necessary include chiral synthesis from a suitable optically pure precursor or resolution of the racemate (or the racemate of a salt or derivative) using, for example, chiral high pressure liquid chromatography (HPLC). Thus, chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and for specific examples, 0 to 5% by volume of an alkylamine e.g. 0.1% diethylamine. Concentration of the eluate affords the enriched mixture.

Alternatively, the racemate (or a racemic precursor) may be reacted with a suitable optically active compound, for example, an alcohol, or, in the case where the compound of the invention contains an acidic or basic moiety, a base or acid such as 1-phenylethylamine or tartaric acid. The resulting diastereomeric mixture may be separated by chromatography and/or fractional crystallisation and one or both of the diastereoisomers converted to the corresponding pure enantiomer(s) by means well known to a skilled person.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see for example, “Stereochemistry of Organic Compounds” by E. L. Eliel and S. H. Wilen (Wiley, 1994).

It is to be understood that the present invention encompasses all isomeric forms and mixtures thereof that possess PLpro inhibitory activity.

Methods for the determination of stereochemistry and the separation of stereoisomers are well-known in the art (see discussion in “Advanced Organic Chemistry”, 7th edition J. March, John Wiley and Sons, New York, 2013).

Compounds of the Formula (I) containing an amine function may also form Noxides. A reference herein to a compound of the Formula (I) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid); this is described in general textbooks such as Advanced Organic Chemistry, by J. March referred to above. N-oxides can be made in a variety of ways which are known to the skilled person; for example, by reacting the amine compound with m-chloroperoxybenzoic acid (mCPBA) in a solvent such as dichloromethane.

The present invention also encompasses compounds of the invention as defined herein which comprise one or more isotopic substitutions. For example, H may be in any isotopic form, including 1H, 2H(D), and 3H (T); C may be in any isotopic form, including 12C, 13C, and 14C; and O may be in any isotopic form, including 16O and 18O; and the like. Similarly, isotopic variants of N, S and P may be utilised.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

The reader's attention is directed to all papers and documents which are filed concurrently with or previous to this specification in connection with this application and which are open to public inspection with this specification, and the contents of all such papers and documents are incorporated herein by reference.

According to another aspect of the present inventions, there is provided a pharmaceutical composition comprising a compound of formula (I), or a pharmaceutically acceptable salt thereof, in association with one or more pharmaceutically acceptable excipients.

Compounds of the invention have been described throughout the present application as a compound or a salt of a compound. It would be understood by the skilled person that a compound can be converted into a salt and a salt can be converted into a compound, in other words the free acid or free base corresponding to the salt. Accordingly, where a compound is disclosed or where a salt is disclosed, the present invention also includes the corresponding salt form, free acid form or free base form, as appropriate.

The compounds of the present invention are inhibitors of PLpro. As discussed above, PLpro plays a key role in viral replication. In particular, PLpro resides within viral polyprotein and is responsible for processing the polyprotein into its functional units. These functional units in turn assemble into complexes to execute viral RNA synthesis. Without wishing to be bound by theory, it is thought that selective inhibition of PLpro can prevent viral replication and can thus be used in the treatment of viral infections.

Viral infections which can be treated using compounds of Formula (I) and compositions containing compounds of Formula (I) may include those caused by coronaviruses, rotaviruses, noroviruses, enteroviruses, hepatitis viruses (e.g. HAV, HBV, HCV), herpesviruses, papillomaviruses, arboviruses (e.g. West Nile virus, Zika virus, Dengue virus), ebolaviruses, rabies virus, or rubella virus. It may be that the viral infection in caused by coronaviruses. For example, the viral infection may be caused by one or more of the following: severe acute respiratory syndrome coronavirus (SARS-CoV), severe acute respiratory syndrome coronavirus 2 (SARS-CoV-2), Middle East respiratory syndrome coronavirus (MERS-CoV), human coronavirus OC43 (HCoV-OC43), human coronavirus HKU1 (HCoV-HKU1), human coronavirus 229E (HCoV-229E), and human coronavirus NL63 (HCoV-NL63).

In one aspect, the present invention provides a compound of formula (I) or pharmaceutically acceptable salt thereof, or a pharmaceutical composition comprising the compound of formula (I) or pharmaceutically acceptable salt thereof for use in the inhibition of PLpro activity.

In a further aspect, the compounds or compositions of the present invention may be for use in a method of treating and/or preventing a disease or disorder caused by coronaviruses, rotaviruses, noroviruses, enteroviruses, hepatitis viruses (e.g. HAV, HBV, HCV, HDV, HEV), herpesviruses, papillomaviruses, arboviruses (e.g. West Nile virus, Zika virus, Dengue virus), ebolaviruses, rabies virus, or rubella virus. It may be that the disease or disorder is selected from: coronavirus disease 2019 (COVID-19), severe acute respiratory syndrome (SARS), Middle East respiratory syndrome (MERS), common cold, other coronavirus infections, gastroenteritis, viral meningitis, polio, hepatitis A, hepatitis B, hepatitis C, hepatitis D, hepatitis E, infectious mononucleosis, human cytomegalovirus, chickenpox, viral warts, oral herpes, genital herpes, HSV encephalitis, West Nile fever, Zika fever, Dengue fever, Japanese encephalitis, tick-borne encephalitis, yellow fever, Ebola virus disease, rabies, and rubella.

It may be that the disease or disorder is caused by coronaviruses. It may be that the disease or disorder is selected from: coronavirus disease 2019 (COVID-19), severe acute respiratory syndrome (SARS), Middle East respiratory syndrome (MERS), common cold, or other coronavirus infections.

The compounds of Formula (I) may be presented in dosage forms which are suitable for oral use (for example as tablets, lozenges, hard or soft capsules, aqueous or oily suspensions, emulsions, dispersible powders or granules, syrups or elixirs), or they may be suitable for topical use (for example as creams, ointments, gels, or aqueous or oily solutions or suspensions). Other suitable dosage forms also include those intended for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing). In a preferred embodiment oral or intravenous administration is preferred, with intravenous administration being most preferred.

Oral dosage formulations may contain, together with the active compound, one or more of the following excipients: diluents, lubricants, binding agents, desiccants, sweeteners, flavourings, colouring agents, wetting agents, and effervescing agents.

Compound of formula (I) are inhibitors of PLpro and the present invention therefore provides a method of inhibiting viral PLpro activity in vitro or in vivo. This method comprises contacting a cell with an effective amount of a compound of formula (I) or a pharmaceutically acceptable salt, hydrate or solvate thereof, or contacting a cell with a pharmaceutical composition comprising a compound of formula (I) or a pharmaceutically acceptable salt thereof.

Accordingly, in one aspect of the invention, there is provided a method of inhibiting viral PLpro activity in vitro or in vivo, the method comprising contacting a cell with an effective amount of a compound of formula (I), or a pharmaceutically acceptable salt thereof; or contacting a cell with a pharmaceutical composition comprising a compound of formula (I) or a pharmaceutically acceptable salt thereof.

In another aspect, the present invention provides a method for the prevention or treatment of viral infection in a patient in need of such treatment, said method comprising administering to said patient a therapeutically effective amount of a compound of formula (I) or a pharmaceutically acceptable salt thereof; or administering to said patient a therapeutically effective amount of a pharmaceutical composition comprising a compound of formula (I) or a pharmaceutically acceptable salt thereof.

In another aspect, the present invention provides a method for the prevention or treatment of a disease or disorder, said method comprising administering to a patient in need of such treatment a therapeutically effective amount of a compound of formula (I) or a pharmaceutically acceptable salt thereof; or administering to a patient in need of such treatment a therapeutically effective amount of a pharmaceutical composition comprising a compound of formula (I) or a pharmaceutically acceptable salt thereof.

It may be that the disease or disorder is selected from: coronavirus disease 2019 (COVID-19), severe acute respiratory syndrome (SARS), Middle East respiratory syndrome (MERS), common cold, other coronavirus infections, gastroenteritis, viral meningitis, polio, hepatitis A, hepatitis B, hepatitis C, hepatitis D, hepatitis E, infectious mononucleosis, human cytomegalovirus, chickenpox, viral warts, oral herpes, genital herpes, HSV encephalitis, West Nile fever, Zika fever, Dengue fever, Japanese encephalitis, tick-borne encephalitis, yellow fever, Ebola virus disease, rabies, and rubella. It may be that the disease or disorder is selected from: coronavirus disease 2019 (COVID-19), severe acute respiratory syndrome (SARS), Middle East respiratory syndrome (MERS), common cold, or other coronavirus infections.

In another aspect, the present invention provides a compound of formula (I), or a pharmaceutically acceptable salt, hydrate or solvate thereof, or a pharmaceutical composition containing a compound of formula (I) or a pharmaceutically acceptable salt thereof, for use in therapy.

In another aspect, the present invention provides a compound of formula (I), or a pharmaceutically acceptable salt thereof, or a pharmaceutical composition containing a compound of formula (I) or a pharmaceutically acceptable salt thereof, for use in the treatment of a viral infection. The treatment may be curative or preventative i.e. prophylactic. Preferably, the treatment is curative; this means that the treatment reduces the overall level of viral infection

Compounds of the invention can be made according to the following general synthetic schemes. Alternatively, compounds of the invention can be made according to or analogously to the methods described below for Examples 1 to 320.

### General Synthetic Schemes

Compound of formula I can be made according to schemes A to C.

Compounds of the invention can be accessed via amines of formula A. In particular, a compound of formula A may be reacted with a compound of formula B in the presence of a coupling agent and a base to provide a compound of formula C, a subset of compounds of the invention.

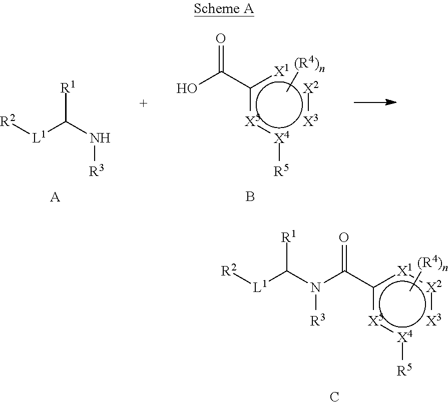

Alternatively, compounds of the invention can be accessed via Scheme B below. In particular, a compound of formula A may be reacted with a compound of formula D in the presence of a coupling agent and a base to provide a compound of formula E. The compound of formula E may be further reacted with a heterocycloalkyl ring a containing an N atom (the ring a being optionally substituted with at least one R⁹ group) in the presence of a coupling agent and a base to provide a compound of formula F, a subset of compounds of the invention.

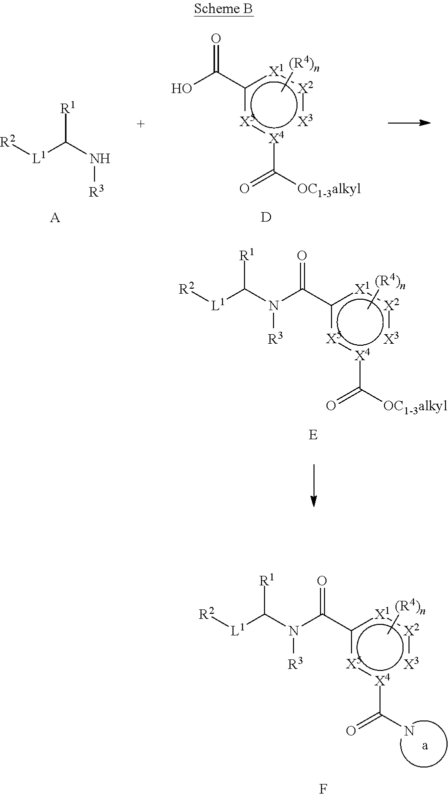

Alternatively, compounds of the invention can be accessed via esters of formula G. In particular, a compound of formula G may be reacted with a compound of formula H in the presence of a palladium catalyst to provide a compound of formula J. Ring b of the compound of formula H may be a phenyl or heteroaryl optionally substituted with at least one R⁸ group or may be a heterocycloalkyl optionally substituted with at least one R⁹ group. The compound of formula J may then be reacted with a compound of formula A in the presence of a coupling agent and a base to provide a compound of formula L, a subset of compounds of the invention.

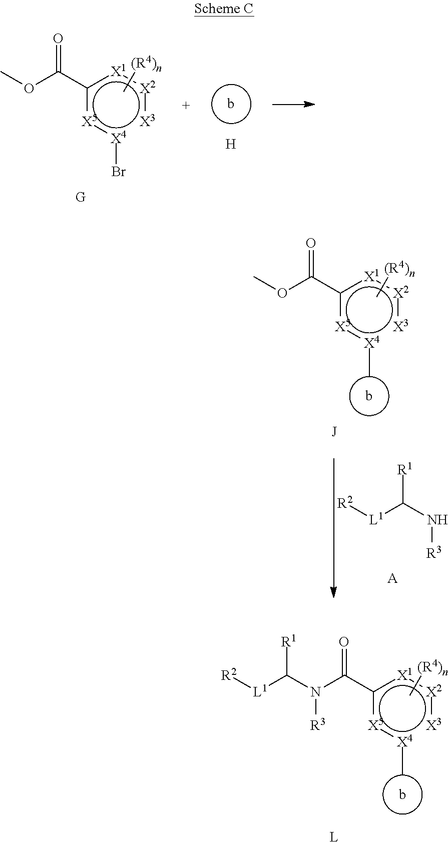

### EXAMPLES

The following compounds represent examples of compounds which can be synthesised in accordance with the invention. Some of the compounds were also tested in a biological assay and the results are presented below. The compounds show activity as inhibitors of papain-like protease (PLpro) and thus have utility in the treatment of viral infections, particularly coronaviruses infections.

### General Experimental

Throughout this document the following abbreviations have been used: DCM—dichloromethane; DIPEA—N,N-diisopropylethylamine; DMF—N,N-dimethylformamide; DMSO—dimethyl sulfoxide; FCC—Flash Column Chromatography; HBTU—N,N,N′,N′-tetramethyl-O-(1H-benzotriazol-1-yl)uronium hexafluorophosphate; THF—tetrahydrofuran; RT—room temperature; R_{T}—retention time; RuPhos—dicyclohexyl(2′,6′-diisopropoxy-[1,1′-biphenyl]-2-yl)phosphine; SCX—Strong Cation Exchange; Xphos Pd G2—chloro(2-dicyclohexylphosphino-2′,4′,6′-tri-iso-propyl-1,1′-biphenyl)[2-(2′-amino-1,1′-biphenyl)]palladium(II).

### Analytical Methods

All ¹H NMR spectra were obtained on a Bruker AVI 500 with 5 mm QNP. Chemical shifts are expressed in parts per million (δ) and are referenced to the solvent Coupling constants J are expressed in Hertz (Hz).

LC-MS were obtained on a Waters Alliance ZQ (Methods A and B) or Waters Acquity H-class UPLC (Method C) using the methods detailed below. Wavelengths were 254 and 210 nm.

### Method A

Column: YMC-Triart C18, 2.0×50 mm, 5 μm. Flow rate: 0.8 mL/min. Injection volume: 6 μL.

Mobile Phase: A=water, B=acetonitrile, C=1:1 water:acetonitrile+1.0% formic acid

| Time | % A | % B | % C |
|---|---|---|---|
| Initial | 90 | 5 | 5 |
| 4.0 | 0 | 95 | 5 |
| 6.0 | 0 | 95 | 5 |

### Method B

Column: YMC-Triart C18, 2.0×50 mm, 5 μm. Flow rate: 0.8 mL/min. Injection volume: 6 μL.

Mobile Phase: A=water, B=acetonitrile, C=1:1 water:acetonitrile+1.0% ammonia (aq.)

| Time | % A | % B | % C |
|---|---|---|---|
| Initial | 90 | 5 | 5 |
| 4.0 | 0 | 95 | 5 |
| 6.0 | 0 | 95 | 5 |

### Method C

Column: CSH C18, 2.1×100 mm, 1.7 μm. Flow rate: 0.6 mL/min. Injection volume: 5 μL Mobile Phase: A=water+0.1% formic acid, B=acetonitrile+0.1% formic acid

| Time | % A | % B |
|---|---|---|
| Initial | 98 | 2 |
| 0.5 | 98 | 2 |
| 6.5 | 2 | 98 |
| 7.5 | 2 | 98 |

### General Procedure 1

The required amine (0.75-1 mmol) was added to DMF (5 mL) and to this was added the required carboxylic acid (1 mmol), HBTU (1 mmol) and DIPEA (3 mmol). The mixture was stirred at RT until complete by LC-MS analysis. The reaction was quenched with water (40 mL), stirred, filtered, and dried to give desired product. On occasions where no precipitate formed on addition of water, the aqueous mixture was extracted with either diethyl ether or ethyl acetate. The organic layer was then washed with brine, dried (Na₂SO₄ or MgSO₄) then filtered and concentrated in vacuo to afford desired product. If necessary, material was then purified via either trituration with a suitable solvent or FCC.

### General Procedure 2

Xphos Pd G2 (0.1 mmol) was added to a degassed solution of the required bromide or triflate (1 mmol), the required boronic acid or pinacol ester (1-1.2 mmol) and potassium phosphate tribasic anhydrous (3 mmol) in 1,4-dioxane (18 mL) and water (2 mL), then the reaction mixture was heated to the required temperature for the required length of time. The reaction mixture was allowed to cool to RT and water (75 mL) and either ethyl acetate (75 mL) or diethyl ether (75 mL) were added. The organic phase was washed with brine (100 mL), dried (Na₂SO₄) and the solvent was removed in vacuo. Purification by FCC gave the desired product.

### General Procedure 3

Palladium(II) acetate (0.1 mmol) was added to a degassed solution of the required aromatic bromide (1 mmol), the required amine (1.2-1.5 mmol), caesium carbonate (2 mmol) and RuPhos (0.2 mmol) in 1,4-dioxane (15 mL) and the reaction mixture heated to the required temperature for the required length of time. The reaction mixture was then allowed to cool to RT and water (75 mL) and either ethyl acetate or diethyl ether (75 mL) added. The phases were separated, and the aqueous phase re-extracted. The combined organic phases were washed with brine (100 mL), dried (Na₂SO₄) and the solvent removed in vacuo. Purification by FCC gave the desired product.

### General Procedure 4

Hydrogen chloride solution (4N in 1,4-dioxane, 10 mL) was added to a solution of the appropriate substrate (0.1 mmol) in DCM (5 mL) at RT and the reaction mixture allowed to stir at this temperature until LC-MS analysis indicated reaction completion. If formation of the hydrochloride salt was possible/desired work-up was via addition of diethyl ether (20 mL), resulting in a white solid which was filtered, triturated with an appropriate solvent, and allowed to dry under vacuum affording desired material. Alternatively, the solvent was removed in vacuo and DCM (80 mL) and a saturated aqueous solution of potassium carbonate (80 mL) added. The phases were then separated, and the organic phase washed with brine (80 mL), dried (Na₂SO₄) and the solvent removed in vacuo. Purification was then either by trituration with an appropriate solvent or by FCC to afford desired product.

### General Procedure 5

Either palladium hydroxide, 20% on carbon or palladium, 10% on activated carbon was added to a solution of the appropriate substrate (0.1 mmol) in MeOH (20 mL) and the reaction mixture evacuated and backfilled with nitrogen (×3), then evacuated and backfilled with hydrogen (×3) and left under hydrogen atmosphere for 2 hours. The reaction mixture was then filtered through Celite, washing with MeOH (150 mL). The solvent was removed in vacuo and purification was either by trituration with an appropriate solvent or by FCC to afford desired product.

### Example 1: N-[(1R)-1-(1-Naphthyl)ethyl]-3-(1-piperidyl)benzamide

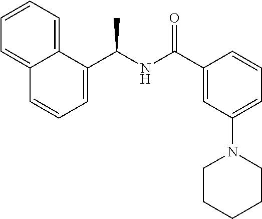 [SMILES: C[C@@H](NC(=O)c1cccc(N2CCCCC2)c1)c1cccc2ccccc12]

Using General Procedure 1 with (1R)-1-(1-Naphthyl)ethanamine (145 mg, 0.85 mmol) and 3-(1-piperidyl)benzoic acid (173 mg, 0.85 mmol) gave N-[(1R)-1-(1-naphthyl)ethyl]-3-(1-piperidyl)benzamide (250 mg, 82%) as a yellow solid. ¹H NMR (500 MHz, CDCl₃) δ 8.18 (d, J=8.5, 1H), 7.87 (dd, J=0.9, 8.2, 1H), 7.85-7.79 (m, 1H), 7.59 (d, J=7.0, 1H), 7.55-7.45 (m, 3H), 7.45-7.35 (m, 1H), 7.25-7.16 (m, 1H), 7.01 (dd, J=2.0, 7.8, 2H), 6.30 (br d, J=7.9, 1H), 6.12 (quin, J=7.1, 1H), 3.22-3.14 (m, 4H), 1.78 (d, J=6.7, 3H), 1.75-1.65 (m, 4H), 1.65-1.54 (m, 2H). LC-MS (Method B): R_{T}=4.25, m/z=357.7 [M−H]⁻.

### Further Examples

The following examples were prepared in a similar manner to N-[(1R)-1-(1-naphthyl)ethyl]-3-(1-piperidyl)benzamide (Example 1), using the required commercially available primary amine and carboxylic acid.

| Example | Structure | Name and Analytical Data |
|---|---|---|
| 2 | 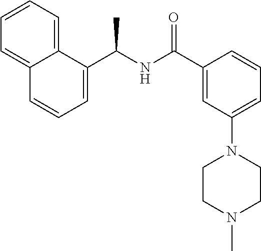 [SMILES: C[C@@H](NC(=O)c1cccc(N2CCN(C)CC2)c1)c1cccc2ccccc12] | 3-(4-Methylpiperazin-1-yl)-N-[(1R)-1-(1- naphthyl)ethyl]benzamide ¹H NMR (500 MHZ, DMSO-d₆) δ 8.88 (d, J = 8.0, 1H), 8.20 (d, J = 8.0, 1H), 7.95 (m, 1H), 7.84 (d, J = 8.0, 1H), 7.64 (d, J = 7.0, 1H), 7.59-7.49 (m, 3H), 7.42 (m, 1H), 7.32 (m, 1H), 7.28 (m, 1H), 7.08 (m, 1H), 5.96 (quin, J = 7.0, 1H), 3.17 (m, 4H), 2.46 (m, 4H), 2.22 (s, 3H), 1.62 (d, J = 7.0, 3H). LC-MS (Method B): R_{T} = 3.59, m/z = 372.8 [M − H]⁻. |
| 3 | 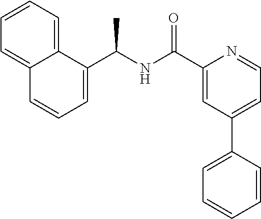 [SMILES: C[C@@H](NC(=O)c1cc(-c2ccccc2)ccn1)c1cccc2ccccc12] | N-[(1R)-1-(1-Naphthyl)ethyl]-4-phenyl-pyridine-2- carboxamide ¹H NMR (500 MHZ, DMSO-d₆) δ 9.22 (d, J = 8.5, 1H), 8.71 (dd, J = 5.0, 0.5, 1H), 8.28 (m, 1H), 8.25 (d, J = 8.5, 1H), 7.96 (m, 1H), 7.93 (dd, J = 5.0, 2.0, 1H), 7.86-7.84 (m, 3H), 7.70 (d, J = 7.0, 1H), 7.60-7.50 (m, 6H), 6.02 (quin, J = 7.0, 1H), 1.70 (d, J = 7.0, 3H). LC-MS (Method B): R_{T} = 4.46, m/z = 353.5 [M + H]⁺. |
| 4 | 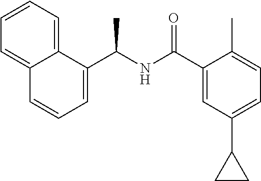 [SMILES: Cc1ccc(C2CC2)cc1C(=O)N[C@H](C)c1cccc2ccccc12] | 5-Cyclopropyl-2-methyl-N-[(1R)-1-(1- naphthyl)ethyl]benzamide ¹H NMR (500 MHZ, DMSO-d₆) δ 8.84 (d, J = 8.0, 1H), 8.24 (d, J = 8.5, 1H), 7.96 (d, J = 8.0, 1H), 7.84 (d, J = 8.0, 1H), 7.63 (m, 1H), 7.60 (m, 1H), 7.56-7.51 (m, 2H), 7.09 (d, J = 8.0, 1H), 7.03 (d, J = 2.0, 1H), 6.99 (dd, J = 8.0, 2.0, 1H), 5.90 (quin, J = 7.0, 1H), 2.23 (s, 3H), 1.91 (m, 1H), 1.59 (d, J = 7.0, 3H), 0.92 (m, 2H), 0.64 (m, 2H). LC-MS (Method B): R_{T} = 4.16, m/z = 328.7 [M − H]⁻. |
| 5 | 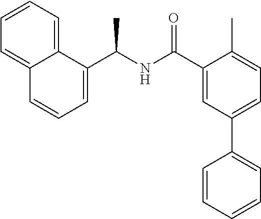 [SMILES: Cc1ccc(-c2ccccc2)cc1C(=O)N[C@H](C)c1cccc2ccccc12] | 2-Methyl-N-[(1R)-1-(1-naphthyl)ethyl]-5-phenyl- benzamide ¹H NMR (500 MHZ, CDCl₃) δ 8.26 (d, J = 8.4, 1H), 7.88 (d, J = 8.1, 1H), 7.81 (d, J = 8.2, 1H), 7.62-7.43 (m, 8H), 7.42-7.36 (m, 2H), 7.35-7.29 (m, 1H), 7.27-7.21 (m, 2H), 6.20-6.11 (m, 1H), 6.05-5.96 (m, 1H), 2.46 (s, 3H), 1.81 (d, J = 6.9, 3H). LC-MS (Method A): R_{T} = 4.25, m/z = 364.7 [M − H]⁻. |

### Example 6: N-[(1R)-1-(1-Naphthyl)ethyl]-3-(4-piperidyl)benzamide Hydrochloride Salt

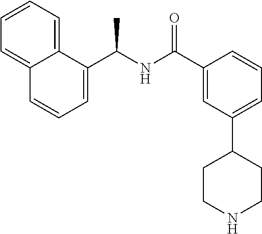 [SMILES: C[C@@H](NC(=O)c1cccc(C2CCNCC2)c1)c1cccc2ccccc12]

tert-Butyl 4-[3-[[(1R)-1-(1-naphthyl)ethyl]carbamoyl]phenyl]piperidine-1-carboxylate (1.50 g, 3.27 mmol)—prepared in a similar manner to N-[(1R)-1-(1-naphthyl)ethyl]-3-(1-piperidyl)benzamide (Example 1)—was added to 6N HCl in propan-2-ol (30 mL) and stirred for 2 hours. The mixture was evaporated to 50% the initial volume. The reaction was diluted with water (50 mL) and extracted with diethyl ether (100 mL). The aqueous was basified with solid NaOH to give a viscous liquid. This was extracted with diethyl ether (2×75 mL), dried (MgSO₄) and solvent removed in vacuo to afford a foamy solid. This was dissolved in the minimum amount of DCM (1 mL) and then diethyl ether (5 mL), the mixture was then acidified with 2.0 N HCl in diethyl ether to afford a solid which was stirred for 20 minutes and then filtered under nitrogen to afford N-[(1R)-1-(1-naphthyl)ethyl]-3-(4-piperidyl)benzamide hydrochloride salt (1.18 g, 89%) as a white solid. ¹H NMR (500 MHz, DMSO-d₆) δ 9.05-8.95 (m, 2H), 8.86-8.73 (m, 1H), 8.21 (d, J=8.2, 1H), 7.98-7.93 (m, 1H), 7.84 (d, J=8.2, 1H), 7.80 (d, J=7.6, 1H), 7.75 (s, 1H), 7.65 (d, J=7.3, 1H), 7.61-7.49 (m, 3H), 7.43 (t, J=7.6, 1H), 7.41-7.37 (m, 1H), 5.97 (quin, J=7.2, 1H), 3.41-3.32 (m, 2H), 3.03-2.86 (m, 3H), 1.97-1.82 (m, 4H), 1.63 (d, J=6.7, 3H). LC-MS (Method B): R_{T}=5.68, m/z=359.6 [M−H]⁻.

### Example 7: 3-(1-Acetyl-4-piperidyl)-N-[(1R)-1-(1-naphthyl)ethyl]benzamide

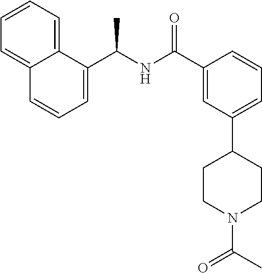 [SMILES: CC(=O)N1CCC(c2cccc(C(=O)N[C@H](C)c3cccc4ccccc34)c2)CC1]

N-[(1R)-1-(1-Naphthyl)ethyl]-3-(4-piperidyl)benzamide hydrochloride salt (Example 6) (159 mg, 402 μmol) was added to DMF (5 mL). To this was added triethylamine (0.13 ml, 925 μmol) then acetyl chloride (31 mg, 403 μmol) to afford a cloudy solution. The mixture was stirred for 10 mins before being quenched with water (40 mL) to afford a solid which was filtered and dried to afford 3-(1-acetyl-4-piperidyl)-N-[(1R)-1-(1-naphthyl)ethyl]benzamide (118 mg, 73%) as a white solid. ¹H NMR (500 MHz, DMSO-d₆) δ 8.92 (br d, J=7.3, 1H), 8.20 (d, J=8.5, 1H), 7.95 (d, J=7.3, 1H), 7.84 (d, J=7.9, 1H), 7.79 (s, 1H), 7.77-7.72 (m, 1H), 7.64 (d, J=7.3, 1H), 7.59-7.49 (m, 3H), 7.42-7.37 (m, 2H), 5.97 (quin, J=7.1, 1H), 4.55 (br s, 1H), 4.05-3.86 (m, 1H), 3.12 (br t, J=12.1, 1H), 2.85-2.78 (m, 1H), 2.61-2.52 (m, 1H), 2.04-2.02 (m, 3H), 1.84-1.74 (m, 2H), 1.63 (d, J=7.1, 4H), 1.54-1.43 (m, 1H). LC-MS (Method B): R_{T}=3.47, m/z=399.8 [M−H]⁻.

### Example 8: 5-Methyl-2-(4-methylpiperazin-1-yl)-N-[(1R)-1-(naphthalen-1-yl)ethyl]pyrimidine-4-carboxamide Hydrochloride Salt

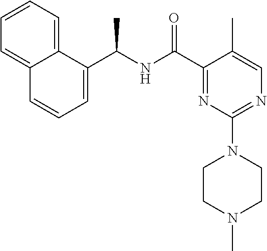 [SMILES: Cc1cnc(N2CCN(C)CC2)nc1C(=O)N[C@H](C)c1cccc2ccccc12]

Step A: 5-Methyl-2-methylsulfanyl-N-[(1R)-1-(1-naphthyl)ethyl]pyrimidine-4-carboxamide Using General Procedure 2 with 2,4,6-trimethyl-1,3,5,2,4,6-trioxatriborinane (187 mg, 1.49 mmol) and 5-bromo-2-methylsulfanyl-N-[(1R)-1-(1-naphthyl) ethyl]pyrimidine-4-carboxamide (1.00 g, 2.49 mmol)—prepared in a similar manner to N-[(1R)-1-(1-naphthyl)ethyl]-3-(1-piperidyl)benzamide (Example 1)—at 100° C. for 4 hours gave 5-methyl-2-methylsulfanyl-N-[(1R)-1-(1-naphthyl)ethyl]pyrimidine-4-carboxamide (330 mg, 39%) as a light yellow gum. LC-MS (Method B): R_{T}=4.26, m/z 336.6=[M−H]⁻.

### Step B: 5-Methyl-2-methylsulfinyl-N-[(1R)-1-(1-naphthyl)ethyl]pyrimidine-4-carboxamide

5-Methyl-2-methylsulfanyl-N-[(1R)-1-(1-naphthyl)ethyl]pyrimidine-4-carboxamide (330 mg, 977 μmol) was dissolved in DCM (20 mL) to this was added 3-chloroperbenzoic acid (265 mg, 1.08 mmol, 70% purity) and the reaction was stirred for 20 mins. The DCM was evaporated to afford a solid, this was quenched with sat K₂CO₃ (30 mL)/water (30 mL), extracted with diethyl ether (2×50 mL), dried (MgSO₄) and solvent removed in vacuo to afford 5-methyl-2-methylsulfinyl-N-[(1R)-1-(1-naphthyl)ethyl]pyrimidine-4-carboxamide (333 mg, 96%) as a yellow gum. This was used directly in Step C.

### Step C: 5-Methyl-2-(4-methylpiperazin-1-yl)-N-[(1R)-1-(naphthalen-1-yl)ethyl] pyrimidine-4-carboxamide Hydrochloride Salt

5-Methyl-2-methylsulfinyl-N-[(1R)-1-(1-naphthyl)ethyl]pyrimidine-4-carboxamide (333 mg, 942 μmol) and N-methylpiperazine (283 mg, 2.83 mmol) were added to DMF (5 mL) and was heated to 50° C. for 5 hours. The reaction was quenched with water to afford a semi solid which was filtered to afford a yellow solid. This was purified by FCC (eluting with ethyl acetate and then 5% 7N NH₃ in ethyl acetate) to afford a clear gum. The gum was dissolved in diethyl ether (1 mL), then 4N HCl in 1,4-dioxane (5 mL) was added to afford a solid which was stirred for 20 mins then filtered under nitrogen to afford 5-methyl-2-(4-methylpiperazin-1-yl)-N-[(1R)-1-(1-naphthyl)ethyl]pyrimidine-4-carboxamide hydrochloride salt (167 mg, 41%) as a yellow grainy solid. ¹H NMR (500 MHz, DMSO-d₆) δ 11.39 (br s, 1H), 9.13 (br s, 1H), 8.43 (br s, 1H), 8.23 (br s, 1H), 7.97 (br s, 1H), 7.91-7.80 (m, 1H), 7.74-7.42 (m, 4H), 5.90 (br s, 1H), 4.72 (br m, 2H), 3.63-3.32 (m, 4H), 3.04 (br m, 2H), 2.77 (br s, 3H), 2.21 (br s, 3H), 1.63 (br s, 3H). LC-MS (Method B): R_{T}=3.94, m/z=388.8 [M−H]⁻.

### Example 9: N-[(1R)-1-(3,4-Dimethoxyphenyl)ethyl]-2-methyl-5-(2-methyl-1,3,3a,4,6,6a-hexahydropyrrolo[3,4-c]pyrrol-5-yl)benzamide

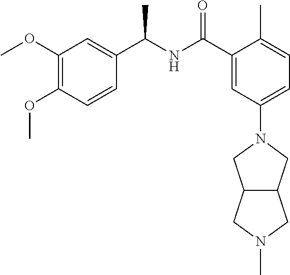 [SMILES: COc1ccc([C@@H](C)NC(=O)c2cc(N3CC4CN(C)CC4C3)ccc2C)cc1OC]

Using General Procedure 3 with 5-bromo-N-[(1R)-1-(3,4-dimethoxyphenyl)ethyl]-2-methyl-benzamide (620 mg, 1.64 mmol)—prepared in a similar manner to N-[(1R)-1-(1-naphthyl)ethyl]-3-(1-piperidyl)benzamide (Example 1)—and 2-methyl-octahydro-pyrrolo[3,4-c]pyrrole (248 mg, 1.97 mmol) at 110° C. overnight with purification by FCC (eluting with 0-50% MeOH in DCM) gave N-[(1R)-1-(3,4-dimethoxyphenyl)ethyl]-2-methyl-5-(2-methyl-1,3,3a,4,6,6a-hexahydropyrrolo[3,4-c]pyrrol-5-yl)benzamide (82 mg, 11%) as a white crystalline solid. ¹H NMR (500 MHz, DMSO-d₆) δ 8.52 (d, J=8.5, 1H), 7.03 (m, 1H), 7.01 (d, J=8.5, 1H), 6.91-6.87 (m, 2H), 6.61 (dd, J=8.0, 2.5, 1H), 6.56 (d, J=2.5, 1H), 5.07 (quin, J=7.0, 1H), 3.76 (s, 3H), 3.74 (s, 3H), 3.04 (m, 2H), 2.87 (m, 2H), 2.38 (m, 2H), 2.21 (s, 3H), 2.16 (s, 3H), 1.40 (d, J=7.0, 3H). LC-MS (Method B): R_{T}=3.62, m/z=422.9 [M−H]⁻.

### Further Examples

The following examples were prepared in a similar manner to N-[(1R)-1-(3,4-dimethoxyphenyl)ethyl]-2-methyl-5-(2-methyl-1,3,3a,4,6,6a-hexahydropyrrolo[3,4-c]pyrrol-5-yl)benzamide (Example 9), using the required aromatic bromide—prepared in a similar manner to N-[(1R)-1-(1-naphthyl)ethyl]-3-(1-piperidyl)benzamide (Example 1) from the required commercially available materials—and the required commercially available secondary amine.

| Example | Structure | Name and Analytical Data |
|---|---|---|
| 10 | 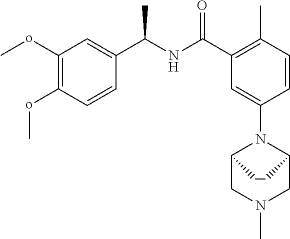 | N-[(1R)-1-(3,4-Dimethoxyphenyl)ethyl]-2- methyl-5-[(1S,5R)-3-methyl-3,8- diazabicyclo[3.2.1]octan-8-yl]benzamide ¹H NMR (500 MHZ, CDCl₃) δ 7.03 (d, J = 8.4, 1H), 6.96-6.89 (m, 2H), 6.87-6.81 (m, 1H), 6.78-6.74 (m, 1H), 6.74-6.67 (m, 1H), 5.87 (d, 1H), 5.33- 5.23 (m, 1H), 4.15-4.06 (m, 2H), 3.92-3.83 (m, 6H), 2.52 (br d, J = 11.0, 2H), 2.41 (d, J = 10.5, 2H), 2.28 (s, 3H), 2.16 (s, 3H), 2.01-1.88 (m, 4H), 1.58 (d, J = 6.9, 3H). LC-MS (Method A): R_{T} = 2.93, m/z = 424.7 [M + H]⁺. |
| 11 | 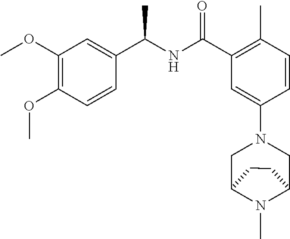 [SMILES: COc1ccc([C@@H](C)NC(=O)c2cc(N3C[C@H]4CC[C@@H](C3)N4C)ccc2C)cc1OC] | N-[(1R)-1-(3,4-Dimethoxyphenyl)ethyl]-2- methyl-5-[(1R,5S)-8-methyl-3,8- diazabicyclo[3.2.1]octan-3-yl]benzamide ¹H NMR (500 MHZ, CDCl₃) δ 7.08-6.99 (m, 1H), 6.96-6.88 (m, 2H), 6.87-6.82 (m, 1H), 6.80-6.76 (m, 1H), 6.75-6.69 (m, 1H), 5.87 (d, 1H), 5.32- 5.23 (m, 1H), 3.89 (s, 3H), 3.86 (s, 3H), 3.33-3.29 (m, 2H), 3.26-3.18 (m, 2H), 2.96 (br d, J = 10.7, 2H), 2.33 (s, 3H), 2.29 (s, 3H), 2.07-1.93 (m, 2H), 1.77-1.69 (m, 2H), 1.58 (d, 3H). LC-MS (Method A): R_{T} = 3.05, m/z = 424.7 [M + H]⁺. |
| 12 | 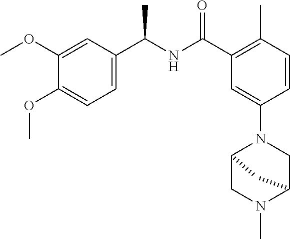 [SMILES: COc1ccc([C@@H](C)NC(=O)c2cc(N3C[C@@H]4C[C@H]3CN4C)ccc2C)cc1OC] | N-[(1R)-1-(3,4-Dimethoxyphenyl)ethyl]-2- methyl-5-[(1S,4S)-5-methyl-2,5- diazabicyclo[2.2.1]heptan-2-yl]benzamide ¹H NMR (500 MHZ, DMSO-d₆) δ 8.48 (d, J = 8.5, 1H), 6.98 (br s, 1H), 6.94 (d, J = 8.0, 1H), 6.86- 6.83 (m, 2H), 6.49 (dd, J = 8.0, 2.5, 1H), 6.41 (d, J = 2.5, 1H), 5.02 (quin, J = 7.0, 1H), 4.19 (br s, 1H), 3.71 (s, 3H), 3.69 (s, 3H), 3.34 (br s, 1H), 3.25 (dd, J = 9.0, 2.0, 1H), 3.07 (d, J = 9.0, 1H), 2.71 (dd, J = 9.0, 2.0, 1H), 2.41 (d, J = 9.0, 1H), 2.18 (s, 3H), 2.09 (s, 3H), 1.80 (br d, J = 9.0, 1H), 1.68 (br d, J = 9.0, 1H), 1.35 (d, J = 7.0, 3H). LC-MS (Method B): R_{T} = 3.46, m/z = 408.9 [M − H]⁻. |
| 13 | 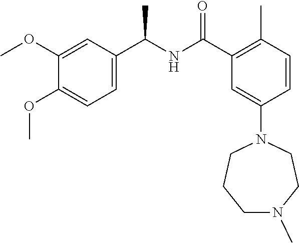 [SMILES: COc1ccc([C@@H](C)NC(=O)c2cc(N3CCCN(C)CC3)ccc2C)cc1OC] | N-[(1R)-1-(3,4-Dimethoxyphenyl)ethyl]-2- methyl-5-(4-methyl-1,4-diazepan-1- yl)benzamide ¹H NMR (500 MHZ, DMSO-d₆) δ 8.52 (d, J = 8.0, 1H), 7.03 (br s, 1H), 6.98 (d, J = 8.5, 1H), 6.89 (m, 2H), 6.65 (dd, J = 8.5, 3.0, 1H), 6.57 (d, J = 3.0, 1H), 5.07 (quin, J = 7.0, 1H), 3.76 (s, 3H), 3.74 (s, 3H), 3.48 (m, 2H), 3.41 (t, J = 6.5, 2H), 2.59 (m, 2H), 2.44 (m, 2H), 2.25 (s, 3H), 2.14 (s, 3H), 1.88 (dt, J = 11.0, 6.0, 2H), 1.40 (d, J = 7.0, 3H). LC-MS (Method B): R_{T} = 3.69, m/z = 410.8 [M − H]⁻. |
| 14 | 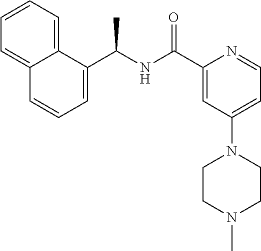 [SMILES: C[C@@H](NC(=O)c1cc(N2CCN(C)CC2)ccn1)c1cccc2ccccc12] | 4-(4-Methylpiperazin-1-yl)-N-[(1R)-1-(1- naphthyl)ethyl]pyridine-2-carboxamide ¹H NMR (500 MHZ, DMSO-d₆) δ ) 8.94 (d, J = 8.5, 1H), 8.22 (d, J = 8.5, 1H), 8.19 (d, J = 6.0, 1H), 7.95 (m, 1H), 7.85 (d, J = 8.0, 1H), 7.65 (d, J = 7.0, 1H), 7.59-7.48 (m, 3H), 7.44 (d, J = 3.0, 1H), 6.99 (dd, J = 6.0, 3.0, 1H), 5.95 (quin, J = 7.0, 1H), 3.37 (m, 4H), 2.41 (m, 4H), 2.21 (s, 3H), 1.65 (d, J = 7.0, 3H). LC-MS (Method B): R_{T} = 3.71, m/z = 373.8 [M − H]⁻. |
| 15 | 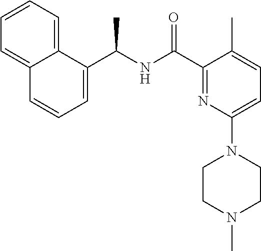 [SMILES: Cc1ccc(N2CCN(C)CC2)nc1C(=O)N[C@H](C)c1cccc2ccccc12] | 3-Methyl-6-(4-methylpiperazin-1-yl)-N-[(1R)-1- (1-naphthyl)ethyl]pyridine-2-carboxamide hydrochloride ¹H NMR (500 MHZ, DMSO-d₆) δ 11.09 (br s, 1H), 8.81 (d, J = 8.0, 1H), 8.25 (d, J = 8.0, 1H), 7.96 (d, J = 8.0, 1H), 7.84 (d, J = 8.0, 1H), 7.65 (d, J = 7.0, 1H), 7.60 (m, 1H), 7.55 (d, J = 8.5, 2H), 7.51 (m, 1H), 7.06 (d, J = 8.5, 1H), 5.89 (quin, J = 7.0, 1H), 4.41 (br d, J = 14.0, 2H), 3.48 (br d, J = 11.5, 2H), 3.28 (br t, J = 13.0, 2H), 3.05 (m, 2H), 2.80 (d, J = 5.0, 3H), 2.34 (s, 3H), 1.63 (d, J = 7.0, 3H). LC- MS (Method B): R_{T} = 4.31, m/z = 387.7 [M − H]⁻. |
| 16 | 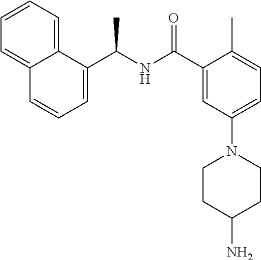 [SMILES: Cc1ccc(N2CCC(N)CC2)cc1C(=O)N[C@H](C)c1cccc2ccccc12] | 5-(4-Amino-1-piperidyl)-2-methyl-N-[(1R)-1-(1- naphthyl)ethyl]benzamide ¹H NMR (500 MHZ, DMSO-d₆) δ 8.85 (d, J = 7.9, 1H), 8.30 (d, J = 8.4, 1H), 8.02 (d, J = 7.9, 1H), 7.90 (d, J = 8.2, 1H), 7.70-7.55 (m, 4H), 7.09 (d, J = 8.4, 1H), 6.94 (dd, J = 2.4, 8.4, 1H), 6.91-6.89 (m, 1H), 5.95 (quin, J = 7.1, 1H), 3.67-3.58 (m, 2H), 2.85- 2.65 (m, 5H), 2.22 (s, 3H), 1.87-1.80 (m, 2H), 1.64 (d, J = 6.9, 3H), 1.46-1.34 (m, 2H). LC-MS (Method A): R_{T} = 3.49, m/z = 388.7 [M + H]⁺. |

### Example 17: 5-(2,6-Diazaspiro[3.3]heptan-2-yl)-2-methyl-N-[(1R)-1-(1-naphthyl)ethyl]benzamide

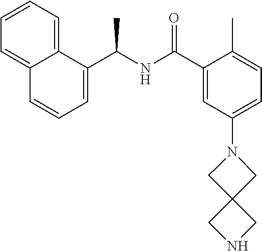 [SMILES: Cc1ccc(N2CC3(CNC3)C2)cc1C(=O)N[C@H](C)c1cccc2ccccc12]

tert-Butyl 6-[4-methyl-3-[[(1R)-1-(1-naphthyl)ethyl]carbamoyl]phenyl]-2,6-diazaspiro[3.3]heptane-2-carboxylate (0.49 g, 1.01 mmol)—prepared in a similar manner to 4-(4-methylpiperazin-1-yl)-N-[(1R)-1-(1-naphthyl)ethyl]pyridine-2-carboxamide (Example 14)—was dissolved in DCM (3 mL) and to this was added trifluoroacetic acid (747 μL, 10.1 mmol) dropwise over one minute giving a clear red solution which was stirred overnight at RT. The reaction mixture was poured directly on to a pre-equilibrated (MeOH) 5 g SCX cartridge and eluted with MeOH followed by 1N NH₃ in MeOH, affording 5-(2,6-diazaspiro[3.3]heptan-2-yl)-2-methyl-N-[(1R)-1-(1-naphthyl)ethyl]benzamide (248 mg, 61%) as a pale yellow powder. ¹H NMR (500 MHz, CDCl₃) δ 8.23 (d, J=8.4, 1H), 7.88 (d, J=7.8, 1H), 7.85-7.78 (m, 1H), 7.61-7.49 (m, 3H), 7.48-7.44 (m, 1H), 6.98 (d, J=9.2, 1H), 6.40-6.34 (m, 2H), 6.11 (quin, J=7.1, 1H), 5.94 (br d, J=8.1, 1H), 3.92-3.79 (m, 4H), 3.75 (s, 4H), 2.28 (s, 3H), 1.83-1.75 (m, 3H). LC-MS (Method A): R_{T}=3.45, m/z=not visible.

### Example 18: 5-[(1S,4S)-2,5-Diazabicyclo[2.2.1]heptan-2-yl]-N-[(1R)-1-(3,4-dimethoxyphenyl)ethyl]-2-methyl-benzamide

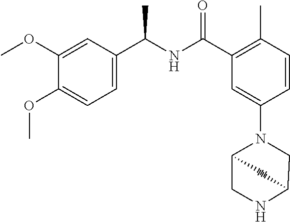

Using General Procedure 4 with tert-butyl (1S,4S)-5-[3-[[(1R)-1-(3,4-dimethoxyphenyl)ethyl]carbamoyl]-4-methyl-phenyl]-2,5-diazabicyclo[2.2.1]heptane-2-carboxylate (170 mg, 343 μmol)—prepared in a similar manner to N-[(1R)-1-(3,4-dimethoxyphenyl)ethyl]-2-methyl-5-[(1S,4S)-5-methyl-2,5-diazabicyclo[2.2.1]heptan-2-yl]benzamide (Example 12)—gave 5-[(1S,4S)-2,5-diazabicyclo[2.2.1]heptan-2-yl]-N-[(1R)-1-(3,4-dimethoxyphenyl)ethyl]-2-methyl-benzamide (81 mg, 57%) as a white crystalline solid. ¹H NMR (500 MHz, DMSO-d₆) δ 8.52 (d, J=8.5, 1H), 7.03 (s, 1H), 6.99 (d, J=8.0, 1H), 6.91-6.87 (m, 2H), 6.53 (dd, J=8.0, 2.5, 1H), 6.45 (d, J=2.5, 1H), 5.06 (quin, J=7.0, 1H), 4.31 (s, 1H), 3.75 (s, 3H), 3.73 (s, 3H), 3.65 (br s, 1H), 3.57 (s, 1H), 3.48 (dd, J=8.5, 2.0, 1H), 2.87 (d, J=9.0, 2H), 2.82 (d, J=9.0, 1H), 2.14 (s, 3H), 1.78 (br d, J=9.0, 1H), 1.65 (br d, J=9.0, 1H), 1.40 (d, J=7.0, 3H). LC-MS (Method B): R_{T}=3.54, m/z=394.8 [M−H]⁻.

### Example 19: 5-[(3S)-3-Isopropylpiperazin-1-yl]-2-methyl-N-[(1R)-1-(1-naphthyl)ethyl]benzamide

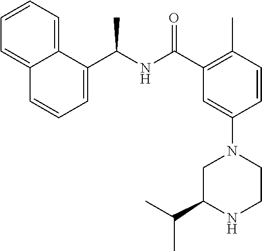 [SMILES: Cc1ccc(N2CCN[C@@H](C(C)C)C2)cc1C(=O)N[C@H](C)c1cccc2ccccc12]

tert-Butyl (2S)-2-isopropyl-4-[4-methyl-3-[[(1R)-1-(1-naphthyl)ethyl]carbamoyl]phenyl]piperazine-1-carboxylate (91 mg, 176 μmol)—4-(4-methylpiperazin-1-yl)-N-[(1R)-1-(1-naphthyl)ethyl]pyridine-2-carboxamide (Example 14)—was dissolved into DCM (2 mL), then HCl in propan-2-ol (6M, 294 μL) added. Mixture was then stirred at RT overnight. The reaction was then concentrated in vacuo. Residue was then dissolved into MeOH, then loaded onto a SCX2 cartridge, which was washed with MeOH, then the product was eluted with 1M NH₃/MeOH. Volatiles were then removed in vacuo. Material was then triturated with diethyl ether, and the solid material isolated to afford 5-[(3S)-3-isopropylpiperazin-1-yl]-2-methyl-N-[(1R)-1-(1-naphthyl)ethyl]benzamide (7 mg, 9%) as an off white powder. ¹H NMR (500 MHz, CDCl₃) δ 8.23 (d, J=8.4, 1H), 7.93-7.79 (m, 2H), 7.63-7.42 (m, 4H), 7.06 (s, 1H), 6.84 (s, 2H), 6.19-6.08 (m, 1H), 5.99-5.88 (m, 1H), 3.57-3.35 (m, 3H), 3.07 (br d, J=7.8, 5H), 2.92-2.66 (m, 3H), 2.31 (s, 3H), 2.12-1.98 (m, 2H), 1.81 (br d, J=6.7, 4H), 1.28-0.91 (m, 12H). LC-MS (Method A): R_{T}=4.51, m/z=416.6 [M+H]⁺.

### Example 20: 5-[6-(2-Hydroxyethyl)-2,6-diazaspiro[3.3]heptan-2-yl]-2-methyl-N-[(1R)-1-(1-naphthyl) ethyl]benzamide

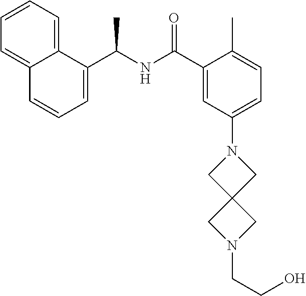 [SMILES: Cc1ccc(N2CC3(CN(CCO)C3)C2)cc1C(=O)N[C@H](C)c1cccc2ccccc12]

5-(2,6-Diazaspiro[3.3]heptan-2-yl)-2-methyl-N-[(1R)-1-(1-naphthyl)ethyl]benzamide (100 mg, 259 μmol) (Example 17), potassium carbonate (103 mg, 747 μmol) and 2-bromoethanol (48.6 mg, 389 μmol) were suspended in ethanol (3.1 mL) and the reaction mixture heated to 85° C. for 18 hours. The reaction mixture was allowed to cool to RT, then water (20 mL) and ethyl acetate (50 mL) were added. The organic phase was washed with brine (10 mL), dried (Na₂SO₄) and the solvent was removed in vacuo. Purification by FCC (eluting with 0-10% 1M NH3 in MeOH in DCM) gave 5-[6-(2-hydroxyethyl)-2,6-diazaspiro[3.3]heptan-2-yl]-2-methyl-N-[(1R)-1-(1-naphthyl)ethyl]benzamide (28 mg, 24%) as a white foam. ¹H NMR (500 MHz, CDCl₃) δ 8.23 (d, J=8.2, 1H), 7.92-7.85 (m, 1H), 7.84-7.76 (m, 1H), 7.60-7.49 (m, 3H), 7.49-7.41 (m, 1H), 7.02-6.93 (m, 1H), 6.41-6.31 (m, 2H), 6.17-6.05 (m, 1H), 5.95-5.84 (m, 1H), 3.89-3.80 (m, 4H), 3.52 (t, 2H), 3.41-3.29 (m, 4H), 2.56 (t, 2H), 2.26 (s, 3H), 1.79 (d, 3H). LC-MS (Method A): R_{T}=3.44, m/z=430.6 [M+H]⁺.

### Example 21: 2-Methyl-5-(4-methylpiperazin-1-yl)-N-[(1R)-1-(1-naphthyl)ethyl]benzamide

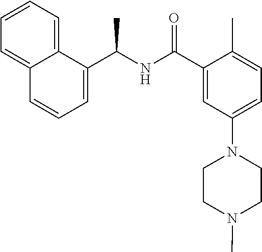 [SMILES: Cc1ccc(N2CCN(C)CC2)cc1C(=O)N[C@H](C)c1cccc2ccccc12]

### Step A: Methyl 2-methyl-5-(4-methylpiperazin-1-yl)benzoate

Using General Procedure 3 with 1-methylpiperazine (6.75 mL, 60.9 mmol) and methyl 5-bromo-2-methyl-benzoate (9.30 g, 40.6 mmol) at 100° C. for 2 hours gave methyl 2-methyl-5-(4-methylpiperazin-1-yl)benzoate (9.36 g, 93%) as an orange oil. LC-MS (Method B): R_{T}=3.20, m/z=249.5 [M+H]⁺.

### Step B: 2-Methyl-5-(4-methylpiperazin-1-yl)benzoic Acid Hydrochloride Salt

Lithium hydroxide monohydrate (1.74 g, 41.5 mmol) was added to a solution of methyl 2-methyl-5-(4-methylpiperazin-1-yl)benzoate (9.36 g, 37.7 mmol) in water (20 mL) and THF (20 mL) and the reaction mixture was heated to 60° C. overnight. The reaction mixture was cooled to RT, then diethyl ether (70 mL) and water (70 mL) were added. The phases were separated, and the aqueous phase was acidified with 2N HCl and the water then removed in vacuo to give an off-white solid. This was filtered and washed with diethyl ether, then dried to give 2-methyl-5-(4-methylpiperazin-1-yl)benzoic acid hydrochloride salt (8.00 g, 78%) as an off-white solid. ¹H NMR (500 MHz, DMSO-d₆) δ 11.45 (br s, 1H), 7.39 (d, J=3.0, 1H), 7.19 (d, J=8.5, 1H), 7.11 (dd, J=8.5, 3.0, 1H), 3.77 (m, 2H), 3.44 (m, 2H), 3.17-3.09 (m, 4H), 2.77 (s, 3H), 2.41 (m, 3H).

### Step C: 2-Methyl-5-(4-methylpiperazin-1-yl)-N-[(1R)-1-(1-naphthyl)ethyl]benzamide

Using General Procedure 1 with (1R)-1-(1-naphthyl)ethanamine (47 mg, 274 μmol) and 2-methyl-5-(4-methylpiperazin-1-yl)benzoic acid hydrochloride salt (71 mg, 302 μmol) with purification by FCC (eluting with 0-50% MeOH in DCM) gave 2-methyl-5-(4-methylpiperazin-1-yl)-N-[(1R)-1-(1-naphthyl)ethyl]benzamide (50 mg, 45%) as a white crystalline solid. ¹H NMR (500 MHz, DMSO-d₆) δ 8.80 (d, J=8.0, 1H), 8.24 (d, J=8.5, 1H), 7.97 (m, 1H), 7.84 (d, J=8.0, 1H), 7.63-7.58 (m, 2H), 7.56-7.50 (m, 2H), 7.05 (d, J=8.5, 1H), 6.90 (dd, J=8.5, 3.0, 1H), 6.85 (d, J=3.0, 1H), 5.89 (quin, J=7.0, 1H), 3.09 (m, 4H), 2.44 (m, 4H), 2.22 (s, 3H), 2.17 (s, 3H), 1.58 (d, J=7.0, 3H). LC-MS (Method B): R_{T}=3.52, m/z=386.8 [M−H]⁻.

### Further Examples

The following examples were prepared in a similar manner to 2-methyl-5-(4-methylpiperazin-1-yl)-N-[(1R)-1-(1-naphthyl)ethyl]benzamide (Example 21), using the required commercially available secondary amine in Step A, and the required commercially available amine in Step C.

| Example | Structure | Name and Analytical Data |
|---|---|---|
| 22 | 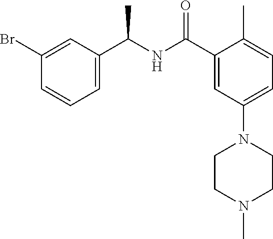 [SMILES: Cc1ccc(N2CCN(C)CC2)cc1C(=O)N[C@H](C)c1cccc(Br)c1] | N-[(1R)-1-(3-Bromophenyl)ethyl]-2-methyl-5-(4- methylpiperazin-1-yl)benzamide ¹H NMR (500 MHZ, DMSO-d₆) δ 8.68 (d, J = 8.0, 1H), 7.60 (m, 1H), 7.44 (m, 1H), 7.40 (m, 1H), 7.31 (m, 1H), 7.06 (d, J = 8.5, 1H), 6.91 (dd, J = 8.5, 3.0, 1H), 6.85 (d, J = 3.0, 1H), 5.08 (quin, J = 7.0, 1H), 3.11 (m, 4H), 2.46 (m, 4H), 2.23 (s, 3H), 2.16 (s, 3H), 1.42 (d, J = 7.0, 3H). LC-MS (Method B): R_{T} = 3.49, m/z = 414.7/416.7 [M − H]⁻. |
| 23 | 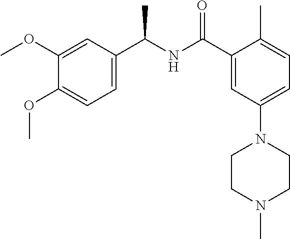 [SMILES: COc1ccc([C@@H](C)NC(=O)c2cc(N3CCN(C)CC3)ccc2C)cc1OC] | N-[(1R)-1-(3,4-Dimethoxyphenyl)ethyl]-2-methyl- 5-(4-methylpiperazin-1-yl)benzamide ¹H NMR (500 MHZ, DMSO-d₆) δ 8.55 (d, J = 8.5, 1H), 7.06 (d, J = 8.5, 1H), 6.92-6.87 (m, 3H), 6.84-6.81 (m, 2H), 5.10-5.03 (m, 1H), 3.76 (s, 3H), 3.70 (s, 3H), 3.14-3.06 (m, 4H), 2.53-2.48 (m, 4H), 2.22 (s, 3H), 2.20-2.14 (m, 3H), 1.40 (d, J = 7.0, 3H). LC-MS (Method B): R_{T} = 2.88, m/z = 396.8 [M − H]⁻. |
| 24 | 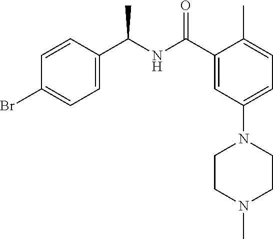 [SMILES: Cc1ccc(N2CCN(C)CC2)cc1C(=O)N[C@H](C)c1ccc(Br)cc1] | N-[(1R)-1-(4-Bromophenyl)ethyl]-2-methyl-5-(4- methylpiperazin-1-yl)benzamide ¹H NMR (500 MHZ, DMSO-d₆) δ 8.66 (d, J = 8.0, 1H), 7.54 (m, 2H), 7.35 (m, 2H), 7.06 (d, J = 8.0, 1H), 6.90 (dd, J = 8.5, 3.0, 1H), 6.84 (d, J = 3.0, 1H), 5.06 (quin, J = 7.0, 1H), 3.10 (m, 4H), 2.45 (m, 4H), 2.22 (s, 3H), 2.15 (s, 3H), 1.41 (d, J = 7.0, 3H). LC-MS (Method B): R_{T} = 3.46, m/z = 414.7/416.7 [M − H]⁻. |
| 25 | 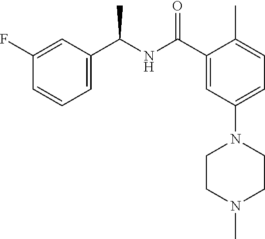 [SMILES: Cc1ccc(N2CCN(C)CC2)cc1C(=O)N[C@H](C)c1cccc(F)c1] | N-[(1R)-1-(3-Fluorophenyl)ethyl]-2-methyl-5-(4- methylpiperazin-1-yl)benzamide ¹H NMR (500 MHZ, DMSO-d₆) δ 8.66 (d, J = 8.0, 1H), 7.38 (m, 1H), 7.24-7.19 (m, 2H), 7.08-7.04 (m, 2H), 6.91 (dd, J = 8.5, 3.0, 1H), 6.85 (d, J = 3.0, 1H), 5.11 (quin, J = 7.0, 1H), 3.11 (m, 4H), 2.45 (m, 4H), 2.22 (s, 3H), 2.16 (s, 3H), 1.42 (d, J = 7.0, 3H). LC-MS (Method B): R_{T} = 3.19, m/z = 354.7 [M − H]⁻. |
| 26 | 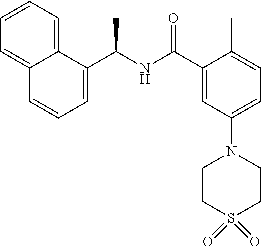 [SMILES: Cc1ccc(N2CCS(=O)(=O)CC2)cc1C(=O)N[C@H](C)c1cccc2ccccc12] | 5-(1,1-Dioxo-1,4-thiazinan-4-yl)-2-methyl-N-[(1R)- 1-(1-naphthyl)ethyl]benzamide ¹H NMR (500 MHZ, CDCl₃) δ 8.23 (d, J = 8.5, 1H), 7.90- 7.88 (m, 1H), 7.83 (d, J = 8.2, 1H), 7.86-7.80 (m, 1H), 7.60-7.55 (m, 2H), 7.55-7.51 (m, 1H), 7.49-7.45 (m, 1H), 7.08 (s, 1H), 6.85-6.78 (m, 2H), 6.13 (quin, J = 7.1, 1H), 6.09-6.08 (m, 1H), 5.89 (br d, J = 8.2, 1H), 5.95- 5.86 (m, 1H), 3.75-3.68 (m, 4H), 3.06-2.99 (m, 4H), 1.81 (d, J = 7.1, 3H). LC-MS (Method B): R_{T} = 3.47, m/z = 421.7 [M − H]⁻. |
| 27 | 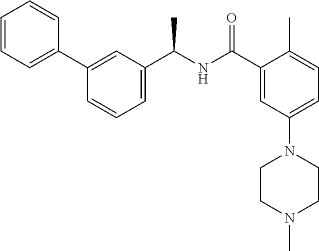 [SMILES: Cc1ccc(N2CCN(C)CC2)cc1C(=O)N[C@H](C)c1cccc(-c2ccccc2)c1] | 2-Methyl-5-(4-methylpiperazin-1-yl)-N-[(1R)-1-(3- phenylphenyl)ethyl]benzamide ¹H NMR (500 MHZ, DMSO-d₆) δ 8.70 (d, J = 8.0, 1H), 7.71 (br s, 1H), 7.66 (m, 2H), 7.54 (d, J = 7.5, 1H), 7.48 (m, 2H), 7.74 (m, 1H), 7.40-7.37 (m, 2H), 7.06 (d, J = 8.5, 1H), 6.91 (dd, J = 8.0, 3.0, 1H), 6.87 (d, J = 3.0, 1H), 5.19 (quin, J = 7.0, 1H), 3.10 (m, 4H), 2.44 (m, 4H), 2.22 (s, 3H), 2.18 (s, 3H), 1.48 (d, J = 7.0, 3H). LC-MS (Method B): R_{T} = 3.79, m/z = 412.8 [M − H]⁻. |
| 28 | 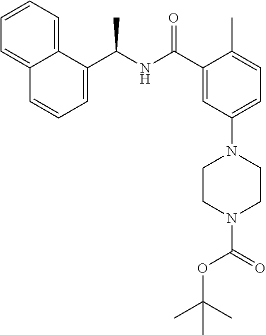 [SMILES: Cc1ccc(N2CCN(C(=O)OC(C)(C)C)CC2)cc1C(=O)N[C@H](C)c1cccc2ccccc12] | tert-Butyl 4-[4-methyl-3-[(1R)-1-(1- naphthyl)ethyl]carbamoyl]phenyl]piperazine-1- carboxylate ¹H NMR (500 MHZ, CDCl₃) δ 8.24 (d, J = 8.4, 1H), 7.88 (d, J = 7.9, 1H), 7.86-7.78 (m, 1H), 7.60-7.54 (m, 2H), 7.54-7.49 (m, 1H), 7.48-7.44 (m, 1H), 7.06 (d, J = 8.4, 1H), 6.88-6.80 (m, 2H), 6.16-6.09 (m, 1H), 5.92 (br d, J = 8.2, 1H), 3.57-3.47 (m, 4H), 3.08-2.94 (m, 4H), 2.31 (s, 3H), 1.80 (d, J = 6.7, 3H), 1.47 (s, 9H). LC-MS (Method A): R_{T} = 4.16, m/z = 472.9 [M − H]⁻. |
| 29 | 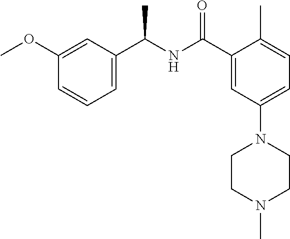 [SMILES: COc1cccc([C@@H](C)NC(=O)c2cc(N3CCN(C)CC3)ccc2C)c1] | N-[(1R)-1-(3-Methoxyphenyl)ethyl]-2-methyl-5-(4- methylpiperazin-1-yl)benzamide ¹H NMR (500 MHZ, CDCl₃) δ 7.30-7.27 (m, 1H), 7.08 (d, J = 8.2, 1H), 6.96 (d, J = 7.6, 1H), 6.92 (d, J = 2.4, 2H), 6.89-6.85 (m, 1H), 6.85-6.79 (m, 1H), 5.92 (br d, J = 7.9, 1H), 5.29 (quin, J = 6.7, 1H), 3.81 (s, 3H), 3.21- 3.11 (m, 4H), 2.60-2.53 (m, 4H), 2.35 (s, 3H), 2.31 (s, 3H), 1.58 (d, J = 6.7, 3H). LC-MS (Method B): R_{T} = 3.12, m/z = 368.6 [M − H]⁺. |
| 30 | 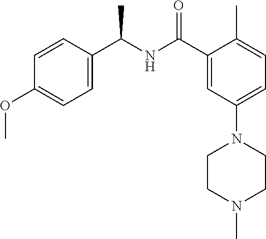 [SMILES: COc1ccc([C@@H](C)NC(=O)c2cc(N3CCN(C)CC3)ccc2C)cc1] | N-[(1R)-1-(4-Methoxyphenyl)ethyl]-2-methyl-5-(4- methylpiperazin-1-yl)benzamide ¹H NMR (500 MHZ, DMSO-d₆) δ 8.55 (d, J = 8.5, 1H), 7.30 (m, 2H), 7.05 (d, J = 8.5, 1H), 6.89 (m, 3H), 6.82 (d, J = 3.0, 1H), 5.06 (quin, J = 7.0, 1H), 3.74 (s, 3H), 3.10 (m, 4H), 2.44 (m, 4H), 2.22 (s, 3H), 2.15 (s, 3H), 1.40 (d, J = 7.0, 3H). LC-MS (Method B): R_{T} = 3.26, m/z = 366.7 [M − H]⁻. |
| 31 | 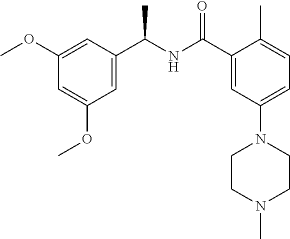 [SMILES: COc1cc(OC)cc([C@@H](C)NC(=O)c2cc(N3CCN(C)CC3)ccc2C)c1] | N-[(1R)-1-(3,5-Dimethoxyphenyl)ethyl]-2-methyl- 5-(4-methylpiperazin-1-yl)benzamide ¹H NMR (500 MHZ, DMSO-d₆) δ 8.59 (d, J = 8.2, 1H), 7.06 (d, J = 8.4, 1H), 6.91 (dd, J = 2.7, 8.4, 1H), 6.84 (d, J = 2.6, 1H), 6.57 (d, J = 2.1, 2H), 6.36 (t, J = 2.1, 1H), 5.07-5.00 (m, 1H), 3.76-3.72 (m, 6H), 3.15-3.06 (m, 4H), 2.48-2.41 (m, 4H), 2.22 (s, 3H), 2.17 (s, 3H), 1.39 (d, J = 7.0, 3H). LC-MS (Method A): R_{T} = 3.56, m/z = 398.6 [M + H]⁺. |
| 32 | 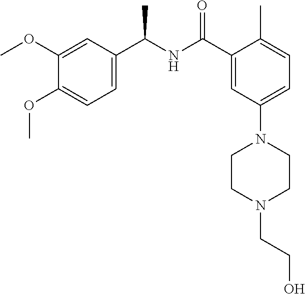 [SMILES: COc1ccc([C@@H](C)NC(=O)c2cc(N3CCN(CCO)CC3)ccc2C)cc1OC] | N-[(1R)-1-(3,4-Dimethoxyphenyl)ethyl]-5-[4-(2- hydroxyethyl)piperazin-1-yl]-2-methyl-benzamide ¹H NMR (500 MHZ, DMSO-d₆) δ 8.59 (d, J = 8.0, 1H), 7.06 (d, J = 8.0, 1H), 7.02 (d, J = 1.5, 1H), 6.91-6.87 (m, 3H), 6.82 (d, J = 3.0, 1H), 5.06 (quin, J = 7.0, 1H), 4.46 (br t, J = 5.0, 1H), 3.75 (s, 3H), 3.73 (s, 3H), 3.53 (q, J = 6.0, 2H), 3.09 (m, 4H), 2.55 (m, 4H), 2.43 (t, J = 6.0, 2H), 2.17 (s, 3H), 1.40 (d, J = 7.0, 3H). LC- MS (Method B): R_{T} = 3.43, m/z = 426.8 [M − H]⁻. |
| 33 | 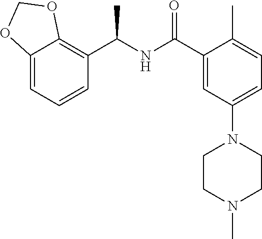 [SMILES: Cc1ccc(N2CCN(C)CC2)cc1C(=O)N[C@H](C)c1cccc2c1OCO2] | N-[(1R)-1-(1,3-Benzodioxol-4-yl)ethyl]-2-methyl-5- (4-methylpiperazin-1-yl)benzamide hydrochloride salt ¹H NMR (500 MHZ, DMSO-d₆) δ 8.59 (br s, 1H), 7.12 (d, J = 8.5, 1H), 7.02-6.93 (m, 2H), 6.90 (s, 1H), 6.88- 6.84 (m, 2H), 6.03-5.95 (m, 2H), 5.04 (s, 1H), 3.89- 3.67 (m, 5H), 3.49 (br s, 2H), 3.12 (br d, J = 9.2, 2H), 2.81 (br s, 2H), 2.18 (s, 3H), 1.39 (d, J = 7.0, 3H). LC- MS (Method B): R_{T} = 3.27 m/z = 380.8 [M − H]⁻. |
| 34 | 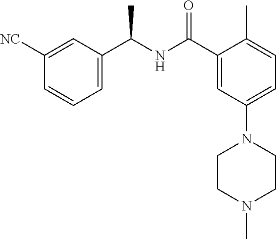 [SMILES: Cc1ccc(N2CCN(C)CC2)cc1C(=O)N[C@H](C)c1cccc(C#N)c1] | N-[(1R)-1-(3-Cyanophenyl)ethyl]-2-methyl-5-(4- methylpiperazin-1-yl)benzamide ¹H NMR (500 MHZ, CDCl₃) δ 7.67 (s, 1H), 7.66-7.62 (m, 1H), 7.58 (d, J = 7.9, 1H), 7.51-7.44 (m, 1H), 7.10 (d, J = 8.2, 1H), 6.94-6.88 (m, 2H), 5.98 (br d, J = 7.3, 1H), 5.31 (quin, J = 7.2, 1H), 3.20-3.14 (m, 4H), 2.60- 2.54 (m, 4H), 2.35 (s, 3H), 2.33-2.28 (m, 3H), 1.61- 1.56 (m, 3H). LC-MS (Method B): R_{T} = 3.03 m/z = 363.6 [M − H]⁻. |
| 35 | 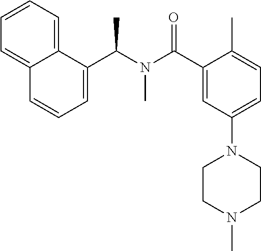 [SMILES: Cc1ccc(N2CCN(C)CC2)cc1C(=O)N(C)[C@H](C)c1cccc2ccccc12] | N,2-Dimethyl-5-(4-methylpiperazin-1-yl)-N-[(1R)-1- (1-naphthyl)ethyl]benzamide ¹H NMR (500 MHZ, DMSO-d₆) δ 8.15 (br s, 1H), 7.99 (d, J = 8.5, 1H), 7.94 (d, J = 8.0, 1H), 7.68 (d, J = 7.0, 1H), 7.63-7.55 (m, 3H), 7.05 (br s, 1H), 6.85 (dd, J = 8.5, 2.0, 1H), 6.58 (m, 1H), 3.06 (m, 4H), 2.25 (br s, 3H), 2.20 (br s, 3H), 1.69 (d, J = 7.0, 3H). LC-MS (Method B): R_{T} = 3.82, m/z = 402.6 [M + H]⁺. |

### Example 36: 5-(2,3,3a,4,6,6a-Hexahydro-1H-pyrrolo[3,4-c]pyrrol-5-yl)-N-[(1R)-1-(3,4-dimethoxyphenyl)ethyl]-2-methyl-benzamide

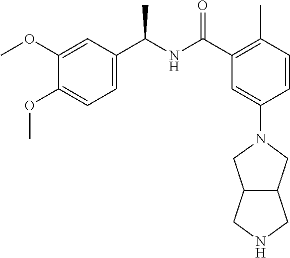 [SMILES: COc1ccc([C@@H](C)NC(=O)c2cc(N3CC4CNCC4C3)ccc2C)cc1OC]

Using General Procedure 4 with tert-butyl 2-[3-[[(1R)-1-(3,4-dimethoxyphenyl)ethyl]carbamoyl]-4-methyl-phenyl]-1,3,3a,4,6,6a-hexahydropyrrolo[3,4-c]pyrrole-5-carboxylate (145 mg, 285 μmol)-prepared in a similar manner to 2-methyl-5-(4-methylpiperazin-1-yl)-N-[(1R)-1-(1-naphthyl)ethyl]benzamide (Example 21)-gave 5-(2,3,3a,4,6,6a-hexahydro-1H-pyrrolo[3,4-c]pyrrol-5-yl)-N-[(1R)-1-(3,4-dimethoxyphenyl)ethyl]-2-methyl-benzamide (64 mg, 52%) as a white crystalline solid after trituration with petroleum ether. ¹H NMR (500 MHz, DMSO-d₆) δ 8.52 (br d, J=8.0, 1H), 7.03 (s, 1H), 7.01 (d, J=8.5, 1H), 6.90 (m, 2H), 6.60 (m, 1H), 6.55 (m, 1H), 5.10 (quin, J=7.0, 1H), 3.76 (s, 3H), 3.74 (s, 3H), 3.58 (s, 2H), 3.01 (br d, J=9.5, 2H), 2.96 (br dd, J=10.5, 6.0, 2H), 2.80 (m, 2H), 2.64 (br d, J=9.5, 2H), 2.16 (s, 3H), 1.40 (d, J=7.0, 3H). LC-MS (Method B): R_{T}=4.30, m/z=408.8 [M−H]⁻.

### Further Examples

The following examples were prepared in a similar manner to 5-(2,3,3a,4,6,6a-hexahydro-1H-pyrrolo[3,4-c]pyrrol-5-yl)-N-[(1R)-1-(3,4-dimethoxyphenyl)ethyl]-2-methyl-benzamide (Example 36).

| Example | Structure | Name and Analytical Data |
|---|---|---|
| 37 | 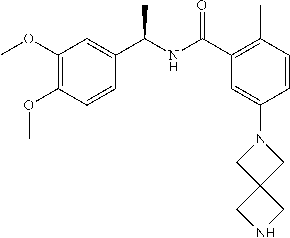 [SMILES: COc1ccc([C@@H](C)NC(=O)c2cc(N3CC4(CNC4)C3)ccc2C)cc1OC] | 5-(2,6-Diazaspiro[3.3]heptan-2-yl)-N-[(1R)-1- (3,4-dimethoxyphenyl)ethyl]-2-methyl- benzamide ¹H NMR (500 MHz, DMSO-d₆) δ 8.55-8.45 (m, 1H), 7.07-6.94 (m, 3H), 6.93-6.85 (m, 2H), 6.43 (dd, J = 2.4, 8.2, 1H), 6.38-6.32 (m, 1H), 5.11-5.02 (m, 1H), 4.22-4.11 (m, 4H), 3.98-3.90 (m, 4H), 3.77 (s, 3H), 3.73 (s, 3H), 2.15 (s, 3H), 1.40 (d, J = 6.9, 3H). LC-MS (Method A): R_{T} = 2.99, m/z = 396.6 [M + H]⁺. |

### Example 38: 2-Methyl-5-(4-methylpiperazin-1-yl)-N-[(1R)-1-(3-methylsulfonylphenyl)ethyl] benzamide

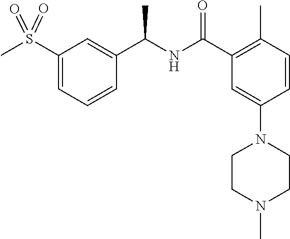 [SMILES: Cc1ccc(N2CCN(C)CC2)cc1C(=O)N[C@H](C)c1cccc(S(C)(=O)=O)c1]

Under an inert atmosphere, N-[(1R)-1-(3-bromophenyl)ethyl]-2-methyl-5-(4-methylpiperazin-1-yl)benzamide (199 mg, 478 μmol) (Example 22), sodium methanesulfinate (73 mg, 717 μmol), copper(I) trifluoromethanesulfonate benzene complex (24 mg, 48 μmol) and (+/−)-trans-1,2-diaminocyclohexane (22 mg, 191 μmol, 23.0 μL) were dissolved in DMSO (1.32 mL) and the reaction mixture heated to 110° C. for 4 hours. The reaction mixture was allowed to cool to RT and 2M potassium carbonate (10 mL) and ethyl acetate (50 mL) were added. The organic phase was washed with brine (100 mL), dried (Na₂SO₄) and the solvent was removed in vacuo. Purification by FCC (eluting with 0-20% 1M NH₃ in MeOH in DCM) gave 2-methyl-5-(4-methylpiperazin-1-yl)-N-[(1R)-1-(3-methylsulfonylphenyl)ethyl]benzamide (31 mg, 15%) as an off white foam. ¹H NMR (500 MHz, CDCl₃-d) δ 8.00-7.93 (m, 1H), 7.85 (d, J=7.8, 1H), 7.69 (d, J=7.6, 1H), 7.61-7.53 (m, 1H), 7.10 (d, J=8.4, 1H), 6.97-6.93 (m, 1H), 6.93-6.86 (m, 1H), 6.09-5.99 (m, 1H), 5.41-5.32 (m, 1H), 3.23-3.13 (m, 4H), 3.04 (s, 3H), 2.60-2.53 (m, 4H), 2.35 (s, 3H), 2.30 (s, 3H), 1.58 (d, 3H). LC-MS (Method A): R_{T}=2.61, m/z=416.6 [M+H]⁺.

### Example 39: 2-Methyl-5-(4-methylpiperazin-1-yl)-N-[(1R)-1-[3-(5-methyl-1,3,4-thiadiazol-2-yl)phenyl]ethyl]benzamide

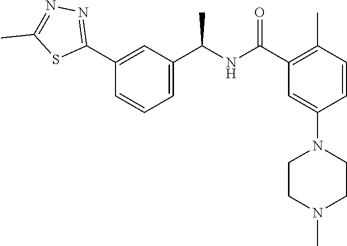 [SMILES: Cc1nnc(-c2cccc([C@@H](C)NC(=O)c3cc(N4CCN(C)CC4)ccc3C)c2)s1]

### Step A: tert-Butyl N-[(1R)-1-(3-bromophenyl)ethyl]carbamate

Di-tert-butyl dicarbonate (2.74 g, 12.5 mmol) was added to a solution of (1R)-1-(3-bromophenyl)ethanamine (2.39 g, 12.0 mmol) in DCM (50 mL) and the reaction mixture allowed to stir at RT overnight. Water (120 mL) and DCM (100 mL) were added, and the phases separated. The aqueous phase was extracted with DCM (100 mL) and the combined organic phases dried over sodium sulfate and the solvent removed in vacuo. Purification by FCC (eluting with 0-50% ethyl acetate in petroleum ether) gave tert-butyl N-[(1R)-1-(3-bromophenyl)ethyl]carbamate (2.50 g, 70%) as a clear oil. ¹H NMR (500 MHz, CDCl₃) δ 7.44 (br s, 1H), 7.36 (m, 1H), 7.23-7.18 (m, 2H), 4.76 (m, 2H), 1.43 (m, 12H).

### Step B: tert-Butyl N-[(1R)-1-[3-(4,4,5,5-tetramethyl-1,3,2-dioxaborolan-2-yl)phenyl]ethyl]carbamate

1,1′-Bis(diphenylphosphino)ferrocenepalladium (II) dichloride (843 mg, 1.15 mmol) was added to a degassed solution of tert-butyl N-[(1R)-1-(3-bromophenyl)ethyl]carbamate (3.46 g, 11.5 mmol), potassium acetate (3.39 g, 34.6 mmol) and bis(pinacolato)diboron (3.51 g, 13.8 mmol) in 1,4-dioxane (15 mL) and the reaction mixture heated at 100° C. for 5 hours. The reaction mixture was allowed to cool to RT overnight. Water (100 mL), brine (50 mL) and ethyl acetate (150 mL) were added, and the phases separated. The organic phase was washed with brine (100 mL), dried over sodium sulfate and the solvent removed in vacuo. Purification by FCC (eluting with 0-25% ethyl acetate in petroleum ether) gave tert-butyl N-[(1R)-1-[3-(4,4,5,5-tetramethyl-1,3,2-dioxaborolan-2-yl)phenyl]ethyl]carbamate (2.00 g, 50%) as a pale yellow oil. ¹H NMR (500 MHz, CDCl₃) δ 7.73 (m, 1H), 7.70 (d, J=7.5, 1H), 7.39 (m, 1H), 7.34 (t, J=7.5, 1H), 4.80 (m, 2H), 1.45 (d, J=6.5, 3H), 1.42 (br s, 9H), 1.35 (s, 12H).

### Step C: tert-Butyl N-[(1R)-1-[3-(5-methyl-1,3,4-thiadiazol-2-yl)phenyl]ethyl]carbamate

Using General Procedure 2 with tert-butyl N-[(1R)-1-[3-(4,4,5,5-tetramethyl-1,3,2-dioxaborolan-2-yl)phenyl]ethyl]carbamate (200 mg, 576 μmol) and 2-bromo-5-methyl-1,3,4-thiadiazole (103 mg, 576 μmol) at 85° C. overnight gave tert-butyl N-[(1R)-1-[3-(5-methyl-1,3,4-thiadiazol-2-yl)phenyl]ethyl]carbamate (75 mg, 41%) as a yellow oil. LC-MS (Method B): R_{T}=3.70, m/z=220.4 [MH-Boc]⁺.

### Step D: (1R)-1-[3-(5-Methyl-1,3,4-thiadiazol-2-yl)phenyl]ethanamine Hydrochloride Salt

Using General Procedure 4 with tert-butyl N-[(1R)-1-[3-(5-methyl-1,3,4-thiadiazol-2-yl)phenyl]ethyl]carbamate (84 mg, 262 μmol) gave (1R)-1-[3-(5-methyl-1,3,4-thiadiazol-2-yl)phenyl]ethanamine hydrochloride salt (67 mg, quant.) as a beige solid. ¹H NMR (500 MHz, DMSO-d₆) δ 8.61 (br s, 3H), 8.16 (br s, 1H), 7.94 (d, J=8.0, 1H), 7.71 (d, J=8.0, 1H), 7.61 (t, J=8.0, 1H), 4.53 (m, 1H), 2.80 (s, 3H), 1.56 (d, J=7.0, 3H).

### Step E: 2-Methyl-5-(4-methylpiperazin-1-yl)-N-[(1R)-1-[3-(5-methyl-1,3,4-thiadiazol-2-yl)phenyl]ethyl]benzamide

Using General Procedure 1 with (1R)-1-[3-(5-methyl-1,3,4-thiadiazol-2-yl)phenyl]ethanamine hydrochloride (67 mg, 306 μmol) and 2-methyl-5-(4-methylpiperazin-1-yl)benzoic acid (79 mg, 336 μmol) (Example 21, Step B) gave 2-methyl-5-(4-methylpiperazin-1-yl)-N-[(1R)-1-[3-(5-methyl-1,3,4-thiadiazol-2-yl)phenyl]ethyl]benzamide (68 mg, 49%) as an off-white solid. ¹H NMR (500 MHz, DMSO-d₆) δ 8.80 (d, J=8.0, 1H), 8.03 (s, 1H), 7.79 (d, J=7.5, 1H), 7.57 (d, J=8.0, 1H), 7.52 (t, J=7.5, 1H), 7.09 (d, J=8.0, 1H), 6.94 (dd, J=8.5, 2.5, 1H), 6.91 (d, J=2.5, 1H), 5.18 (quin, J=7.0, 1H), 3.21 (m, 4H), 2.79 (s, 3H), 2.75 (m, 4H), 2.44 (br s, 3H), 2.18 (s, 3H), 1.48 (d, J=7.0, 3H). LC-MS (Method B): R_{T}=3.49, m/z=434.9 [M−H]⁻.

### Example 40: N-[(1R)-1-[3-(4-Methoxyphenyl)phenyl]ethyl]-2-methyl-5-(4-methylpiperazin-1-yl)benzamide

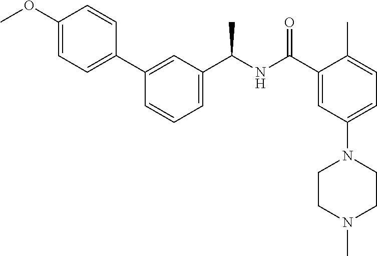 [SMILES: COc1ccc(-c2cccc([C@@H](C)NC(=O)c3cc(N4CCN(C)CC4)ccc3C)c2)cc1]

Using General Procedure 2 with N-[(1R)-1-(3-bromophenyl)ethyl]-2-methyl-5-(4-methylpiperazin-1-yl)benzamide (Example 22) (99 mg, 238 μmol) and (4-methoxyphenyl)boronic acid (40 mg, 262 μmol) at 85° C. for 3.5 hours with purification by FCC (eluting with 0-35% MeOH in DCM) gave N-[(1R)-1-[3-(4-methoxyphenyl)phenyl]ethyl]-2-methyl-5-(4-methylpiperazin-1-yl)benzamide (40 mg, 34%) as a tan crystalline solid. ¹H NMR (500 MHz, DMSO-d₆) δ 8.69 (d, J=8.0, 1H), 7.66 (br s, 1H), 7.60 (d, J=8.5, 2H), 7.48 (m, 1H), 7.39 (t, J=7.5, 1H), 7.32 (d, J=7.5, 1H), 7.06 (d, J=8.5, 1H), 7.03 (d, J=8.5, 2H), 6.91 (dd, J=8.5, 3.0, 1H), 6.86 (d, J=2.5, 1H), 5.17 (quin, J=7.0, 1H), 3.81 (s, 3H), 3.09 (m, 4H), 2.43 (m, 4H), 2.22 (s, 3H), 2.18 (s, 3H), 1.47 (d, J=7.0, 3H). LC-MS (Method B): R_{T}=3.72, m/z=442.9 [M−H]⁻.

### Further Examples

The following examples were prepared in a similar manner to N-[(1R)-1-[3-(4-methoxyphenyl)phenyl]ethyl]-2-methyl-5-(4-methylpiperazin-1-yl)benzamide (Example 40), using the required commercially available boronic ester or acid, with the example intermediate stated.

| Example | | |
|---|---|---|
| (Intermediate) | Structure | Name and Analytical Data |
| 41 (Example 22) | 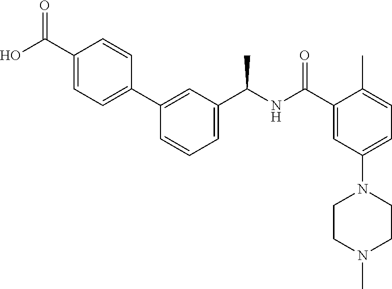 [SMILES: Cc1ccc(N2CCN(C)CC2)cc1C(=O)N[C@H](C)c1cccc(-c2ccc(C(=O)O)cc2)c1] | 4-[3-[(1R)-1-[2-Methyl-5-(4- methylpiperazin-1- yl)benzoyl]amino]ethyl]phenyl]benzoic acid ¹H NMR (500 MHz, DMSO-d₆) δ 8.72 (d, J = 8.0, 1H), 8.05 (d, J = 8.0, 2H), 7.82- 7.79 (m, 3H), 7.62 (m, 1H), 7.50-7.44 (m, 2H), 7.13 (d, J = 8.5, 1H), 6.99 (dd, J = 8.5, 3.0, 1H), 6.92 (d, J = 3.0, 1H), 5.21 (quin, J = 7.5, 1H), 2.85 (s, 3H), 2.20 (s, 3H), 1.49 (d, J = 7.0, 3H). LC-MS (Method B): R_{T} = 1.98, m/z = 456.9 [M − H]⁻. |
| 42 (Example 22) | 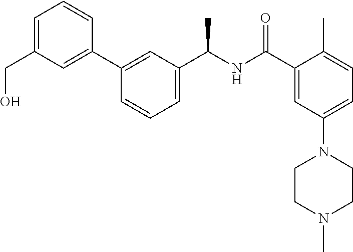 [SMILES: Cc1ccc(N2CCN(C)CC2)cc1C(=O)N[C@H](C)c1cccc(-c2cccc(CO)c2)c1] | N-[(1R)-1-[3-[3- (Hydroxymethyl)phenyl]phenyl]ethyl]- 2-methyl-5-(4-methylpiperazin-1- yl)benzamide ¹H NMR (500 MHz, DMSO-d₆) δ 8.71 (d, J = 8.0, 1H), 7.70 (br s, 1H), 7.61 (br s, 1H), 7.53 (br s, 1H), 7.52 (br s, 1H), 7.45- 7.41 (m, 2H), 7.38 (br d, J = 8.0, 1H), 7.33 (br d, J = 7.5, 1H), 7.06 (d, J = 8.5, 1H), 6.91 (dd, J = 8.0, 3.0, 1H), 6.86 (d, J = 3.0, 1H), 5.25 (br t, J = 4.0, 1H), 5.18 (quin, J = 7.0, 1H), 4.58 (d, J = 4.0, 2H), 3.10 (m, 4H), 2.45 (m, 4H), 2.23 (s, 3H), 2.18 (s, 3H), 1.48 (d, J = 7.0, 3H). LC-MS (Method B): R_{T} = 3.22, m/z = 442.8 [M − H]⁻. |
| 43 (Example 22) | 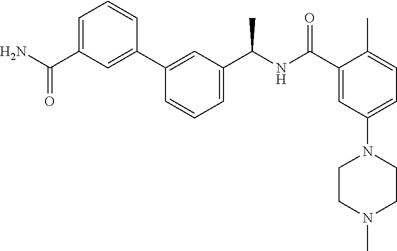 [SMILES: Cc1ccc(N2CCN(C)CC2)cc1C(=O)N[C@H](C)c1cccc(-c2cccc(C(N)=O)c2)c1] | N-[(1R)-1-[3-(3- Carbamoylphenyl)phenyl]ethyl]-2- methyl-5-(4-methylpiperazin-1- yl)benzamide ¹H NMR (500 MHz, DMSO-d₆) δ 8.72 (d, J = 8.0, 1H), 8.17 (br s, 1H), 8.10 (br s, 1H), 7.87 (d, J = 8.0, 1H), 7.81 (m, 1H), 7.77 (m, 1H), 7.60 (d, J = 7.5, 1H), 7.55 (t, J = 8.0, 1H), 7.48-7.41 (m, 3H), 7.06 (d, J = 8.0, 1H), 6.90 (dd, J = 8.5, 2.5, 1H), 6.86 (d, J = 3.0, 1H), 5.20 (quin, J = 7.5, 1H), 3.09 (m, 4H), 2.43 (m, 4H), 2.22 (s, 3H), 2.18 (s, 3H), 1.49 (d, J = 7.5, 3H). LC- MS (Method B): R_{T} = 2.97, m/z = 455.8 [M − H]⁻. |
| 44 (Example 22) | 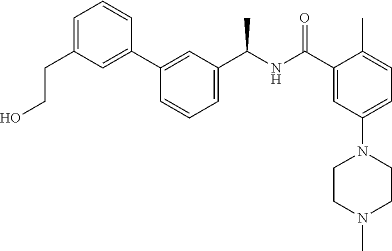 [SMILES: Cc1ccc(N2CCN(C)CC2)cc1C(=O)N[C@H](C)c1cccc(-c2cccc(CCO)c2)c1] | N-[(1R)-1-[3-[3-(2- Hydroxyethyl)phenyl]phenyl]ethyl]-2- methyl-5-(4-methylpiperazin-1- yl)benzamide ¹H NMR (500 MHz, DMSO-d₆) δ 8.71 (d, J = 8.0, 1H), 7.69 (br s, 1H), 7.52-7.47 (m, 3H), 7.43 (t, J = 7.5, 1H), 7.39-7.36 (m, 2H), 7.23 (d, J = 7.5, 1H), 7.07 (d, J = 8.5, 1H), 6.91 (dd, J = 8.5, 2.5, 1H), 6.86 (d, J = 2.5, 1H), 5.18 (quin, J = 7.0, 1H), 4.69 (t, J = 5.0, 1H), 3.66 (m, 2H), 3.10 (m, 4H), 2.80 (t, J = 7.0, 2H), 2.44 (m, 4H), 2.22 (s, 3H), 2.19 (s, 3H), 1.48 (d, J = 7.0, 3H). LC-MS (Method B): R_{T} = 3.67, m/z = 456.8 [M − H]⁻. |
| 45 (Example 22) | 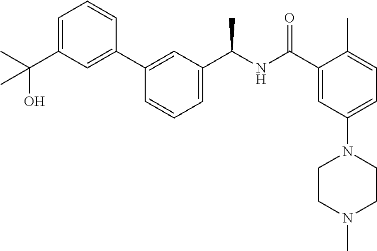 [SMILES: Cc1ccc(N2CCN(C)CC2)cc1C(=O)N[C@H](C)c1cccc(-c2cccc(C(C)(C)O)c2)c1] | N-[(1R)-1-[3-[3-(1-Hydroxy-1-methyl- ethyl)phenyl]phenyl]ethyl]-2-methyl-5- (4-methylpiperazin-1-yl)benzamide ¹H NMR (500 MHz, DMSO-d₆) δ 8.72 (d, J = 8.0, 1H), 7.75 (br s, 1H), 7.68 (br s, 1H), 7.51 (d, J = 7.5, 1H), 7.47-7.38 (m, 5H), 7.06 (d, J = 8.5, 1H), 6.91 (dd, J = 8.5, 2.5, 1H), 6.86 (d, J = 2.5, 1H), 5.19 (quin, J = 7.5, 1H), 5.08 (s, 1H), 3.10 (m, 4H), 2.43 (m, 4H), 2.22 (s, 3H), 2.18 (s, 3H), 1.48 (m, 9H). LC-MS (Method B): R_{T} = 3.60, m/z = 470.9 [M − H]⁻. |
| 46 (Example 22) | 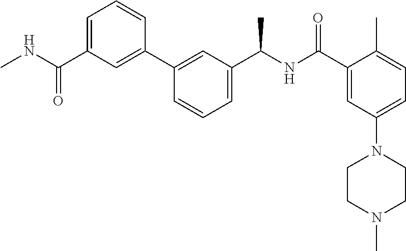 [SMILES: CNC(=O)c1cccc(-c2cccc([C@@H](C)NC(=O)c3cc(N4CCN(C)CC4)ccc3C)c2)c1] | 2-Methyl-N-[(1R)-1-[3-[3- (methylcarbamoyl)phenyl]phenyl]ethyl]- 5-(4-methylpiperazin-1-yl)benzamide ¹H NMR (500 MHz, DMSO-d₆) δ 8.72 (d, J = 8.0, 1H), 8.56 (m, 1H), 8.12 (s, 1H), 7.83-7.80 (m, 2H), 7.76 (br s, 1H), 7.60 (d, J = 7.5, 1H), 7.56 (t, J = 7.5, 1H), 7.47 (t, J = 7.5, 1H), 7.42 (d, J = 7.5, 1H), 7.06 (d, J = 8.5, 1H), 6.91 (dd, J = 8.5, 2.5, 1H), 6.36 (d, J = 2.5, 1H), 5.20 (quin, J = 7.0, 1H), 3.09 (m, 4H), 2.82 (d, J = 4.5, 3H), 2.43 (m, 4H), 2.22 (s, 3H), 2.18 (s, 3H), 1.49 (d, J = 7.0, 3H). LC-MS (Method B): R_{T} = 3.12, m/z = 469.9 [M − H]⁻. |
| 47 (Example 22) | 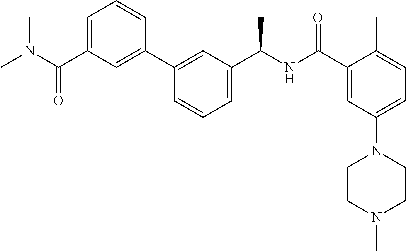 [SMILES: Cc1ccc(N2CCN(C)CC2)cc1C(=O)N[C@H](C)c1cccc(-c2cccc(C(=O)N(C)C)c2)c1] | N-[(1R)-1-[3-[3- (Dimethylcarbamoyl)phenyl]phenyl]ethyl]- 2-methyl-5-(4-methylpiperazin-1- yl)benzamide ¹H NMR (500 MHz, CDCl₃) δ 7.67-7.56 (m, 3H), 7.52-7.43 (m, 3H), 7.42-7.35 (m, 2H), 7.14-7.03 (m, 1H), 6.95-6.91 (m, 1H), 6.91-6.84 (m, 1H), 6.00 (br d, J = 7.9, 1H), 5.39-5.39 (m, 1H), 3.26-3.15 (m, 7H), 3.00 (s, 3H), 2.62-2.54 (m, 4H), 2.36 (s, 3H), 2.34-2.30 (m, 3H), 1.64 (d, J = 6.7, 3H). LC-MS (Method B): R_{T} = 3.27 m/z = 484.0 [M − H]⁻. |
| 48 (Example 22) | 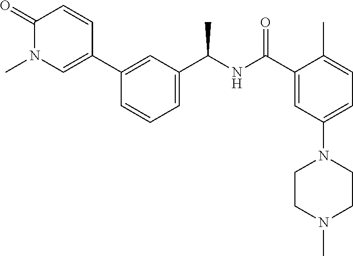 [SMILES: Cc1ccc(N2CCN(C)CC2)cc1C(=O)N[C@H](C)c1cccc(-c2ccc(=O)n(C)c2)c1] | 2-Methyl-N-[(1R)-1-[3-(1-methyl-6-oxo- 3-pyridyl)phenyl]ethyl]-5-(4- methylpiperazin-1-yl)benzamide ¹H NMR (500 MHz, DMSO-d₆) δ 8.64 (d, J = 8.0, 1H), 8.12 (d, J = 3.0, 1H), 7.81 (dd, J = 9.5, 3.0, 1H), 7.60 (m, 1H), 7.45 (m, 1H), 7.39 (t, J = 7.5, 1H), 7.32 (d, J = 7.5, 1H), 7.06 (d, J = 8.5, 1H), 6.91 (dd, J = 8.0, 3.0, 1H), 6.85 (d, J = 3.0, 1H), 6.50 (d, J = 9.5, 1H), 5.16 (quin, J = 7.0, 1H), 3.52 (s, 3H), 3.10 (m, 4H), 2.44 (m, 4H), 2.22 (s, 3H), 2.17 (s, 3H), 1.45 (d, J = 7.0, 3H). LC-MS (Method B): R_{T} = 3.05, m/z = 443.9 [M − H]⁻. |
| 49 (Example 22) | 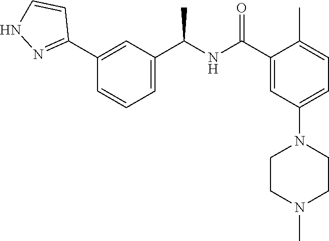 [SMILES: Cc1ccc(N2CCN(C)CC2)cc1C(=O)N[C@H](C)c1cccc(-c2cc[nH]n2)c1] | 2-Methyl-5-(4-methylpiperazin-1-yl)-N- [(1R)-1-[3-(1H-pyrazol-3- yl)phenyl]ethyl]benzamide ¹H NMR (500 MHz, DMSO-d₆) δ 13.31 (br s, 0.25H), 12.87 (br s, 0.75H), 8.59 (m, 1H), 7.88 (br s, 1H), 7.80 (m, 1H), 6.78- 6.72 (m, 1H), 7.43-7.30 (m, 2H), 7.06 (d, J = 8.5, 1H), 6.91 (dd, J = 8.5, 2.5, 1H), 6.87 (d, J = 2.5, 1H), 6.66 (m, 1H), 5.15 (quin, J = 7.0, 1H), 3.11 (m, 4H), 2.45 (m, 4H), 2.22 (s, 3H), 2.18 (s, 3H), 1.46 (d, J = 7.0, 3H). LC-MS (Method B): R_{T} = 3.11, m/z = 402.9 [M − H]⁻. |
| 50 (Example 22) | 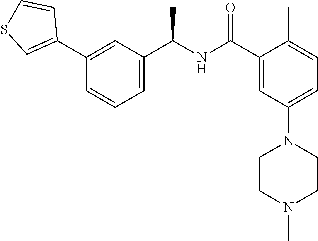 [SMILES: Cc1ccc(N2CCN(C)CC2)cc1C(=O)N[C@H](C)c1cccc(-c2ccsc2)c1] | 2-Methyl-5-(4-methylpiperazin-1-yl)-N- [(1R)-1-[3-(3- thienyl)phenyl]ethyl]benzamide ¹H NMR (500 MHz, DMSO-d₆) δ 8.67 (d, J = 8.0, 1H), 7.84 (dd, J = 3.0, 1.5, 1H), 7.76 (m, 1H), 7.66 (dd, J = 5.0, 3.0, 1H), 7.59 (br d, J = 7.5, 1H), 7.55 (dd, J = 5.0, 1.5, 1H), 7.38 (t, J = 7.5, 1H), 7.32 (br d, J = 7.5, 1H), 7.06 (d, J = 8.0, 1H), 6.91 (dd, J = 8.0, 3.0, 1H), 6.86 (d, J = 3.0, 1H), 5.16 (quin, J = 7.0, 1H), 3.11 (m, 4H), 2.45 (m, 4H), 2.22 (s, 3H), 2.18 (s, 3H), 1.47 (d, J = 7.0, 3H). LC-MS (Method B): R_{T} = 3.75, m/z = 418.8 [M − H]⁻. |
| 51 (Example 22) | 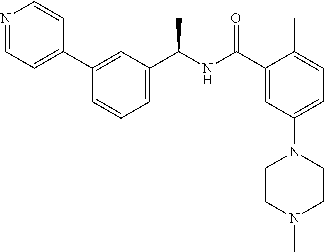 [SMILES: Cc1ccc(N2CCN(C)CC2)cc1C(=O)N[C@H](C)c1cccc(-c2ccncc2)c1] | 2-Methyl-5-(4-methylpiperazin-1-yl)-N- [(1R)-1-[3-(4- pyridyl)phenyl]ethyl]benzamide ¹H NMR (500 MHz, DMSO-d₆) δ 8.71 (d, J = 8.0, 1H), 8.65 (m, 2H), 7.85 (br s, 1H), 7.71 (m, 2H), 7.68 (m, 1H), 7.51 (m, 2H), 7.07 (d, J = 8.5, 1H), 6.91 (dd, J = 8.5, 2.5, 1H), 6.87 (d, J = 2.5, 1H), 5.21 (quin, J = 7.0, 1H), 3.10 (m, 4H), 2.44 (m, 4H), 2.22 (s, 3H), 2.17 (s, 3H), 1.48 (d, J = 7.0, 3H). LC-MS (Method B): R_{T} = 3.23, m/z = 413.9 [M − H]⁻. |
| 52 (Example 22) | 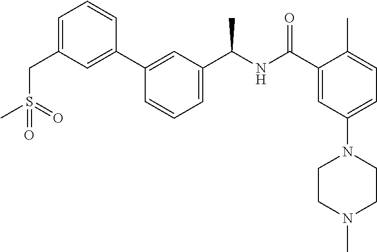 [SMILES: Cc1ccc(N2CCN(C)CC2)cc1C(=O)N[C@H](C)c1cccc(-c2cccc(CS(C)(=O)=O)c2)c1] | 2-Methyl-5-(4-methylpiperazin-1-yl)-N- [(1R)-1-[3-[3 (methylsulfonylmethyl)phenyl]phenyl] ethyl]benzamide ¹H NMR (500 MHz, CDCl₃) δ 7.66-7.56 (m, 3H), 7.53-7.35 (m, 5H), 7.14-7.03 (m, 1H), 6.96-6.84 (m, 2H), 6.03 (br d, J = 7.9, 1H), 5.45-5.25 (m, 1H), 4.31 (s, 2H), 3.19- 3.13 (m, 4H), 2.79 (s, 3H), 2.61-2.51 (m, 4H), 2.39-2.20 (m, 6H), 1.64 (d, J = 6., 3H). LC-MS (Method B R_{T} = 3.45, m/z = 504.9 [M − H]⁻. |
| 53 (Example 22) | 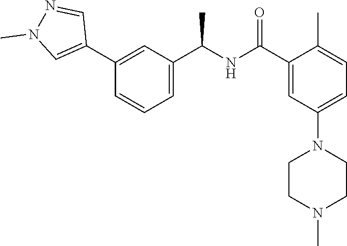 [SMILES: Cc1ccc(N2CCN(C)CC2)cc1C(=O)N[C@H](C)c1cccc(-c2cnn(C)c2)c1] | 2-Methyl-5-(4-methylpiperazin-1-yl)-N- [(1R)-1-[3-(1-methylpyrazol-4- yl)phenyl]ethyl]benzamide ¹H NMR (500 MHz, DMSO-d₆) δ 8.63 (d, J = 8.2, 1H), 8.10 (s, 1H), 7.82 (s, 1H), 7.60 (s, 1H), 7.42 (d, J = 7.6, 1H), 7.32 (t, J = 7.6, 1H), 7.21 (d, J = 7.8, 1H), 7.06 (d, J = 8.4, 1H), 6.91 (dd, J = 2.6, 8.4, 1H), 6.86 (d, J = 2.6, 1H), 5.12 (quin, J = 7.3, 1H), 3.87 (s, 3H), 3.14-3.07 (m, 4H), 2.46-2.43 (m, 4H), 2.22 (s, 3H), 2.17 (s, 3H), 1.45 (d, J = 7.3, 3H). LC-MS (Method B): R_{T} = 3.23, m/z = 418.7 [M + H]⁺. |
| 54 (Example 24) | 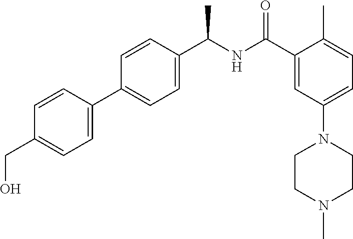 [SMILES: Cc1ccc(N2CCN(C)CC2)cc1C(=O)N[C@H](C)c1ccc(-c2ccc(CO)cc2)cc1] | N-[(1R)-1-[4-[4- (Hydroxymethyl)phenyl]phenyl]ethyl]- 2-methyl-5-(4-methylpiperazin-1- yl)benzamide ¹H NMR (500 MHz, DMSO-d₆) δ 8.67 (d, J = 8.0, 1H), 7.64-7.62 (m, 4H), 7.47 (d, J = 8.0, 2H), 7.40 (d, J = 8.0, 2H), 7.06 (d, J = 8.5, 1H), 6.91 (dd, J = 8.5, 2.5, 1H), 6.87 (d, J = 2.5, 1H), 5.20 (t, J = 6.0, 1H), 5.14 (m, 1H), 4.54 (d, J = 6.0, 2H), 3.11 (m, 4H), 2.46 (m, 4H), 2.22 (s, 3H), 2.18 (s, 3H), 1.46 (d, J = 7.0, 3H). LC-MS (Method B): R_{T} = 3.07, m/z = 442.8 [M − H]⁻. |
| 55 (Example 22) | 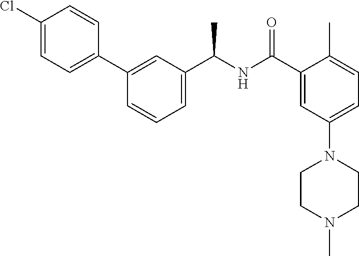 [SMILES: Cc1ccc(N2CCN(C)CC2)cc1C(=O)N[C@H](C)c1cccc(-c2ccc(Cl)cc2)c1] | N-[(1R)-1-[3-(4- Chlorophenyl)phenyl]ethyl]-2-methyl- 5-(4-methylpiperazin-1-yl)benzamide ¹H NMR (500 MHz, CDCl₃) δ 7.60-7.54 (m, 1H), 7.53-7.49 (m, 2H), 7.48-7.36 (m, 5H), 7.09 (d, J = 8.5, 1H), 6.96-6.90 (m, 1H), 6.88 (dd, J = 2.6, 8.4, 1H), 5.98 (br d, J = 7.9, 1H), 5.43-5.34 (m, 1H), 3.19-3.12 (m, 4H), 2.59-2.51 (m, 4H), 2.38-2.29 (m, 6H), 1.72-1.51 (m, 3H). LC-MS (Method A): R_{T} = 4.07, m/z = 446.9 [M − H]⁺. |
| 56 (Example 22) | 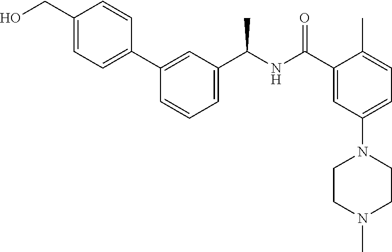 [SMILES: Cc1ccc(N2CCN(C)CC2)cc1C(=O)N[C@H](C)c1cccc(-c2ccc(CO)cc2)c1] | N-[(1R)-1-[3-[4- (Hydroxymethyl)phenyl]phenyl]ethyl]- 2-methyl-5-(4-methylpiperazin-1- yl)benzamide ¹H NMR (500 MHz, CDCl₃) δ 7.62-7.53 (m, 3H), 7.50 (br d, J = 7.9, 1H), 7.47-7.39 (m, 3H), 7.37-7.34 (m, 1H), 7.13-7. 09 (m, 1 H), 6.90-6.84 (m, 2H), 6.05 (br d, J = 7.9, 1H), 5.43-5.35 (m, 1H), 4.73 (s, 2H), 3.12-3.04 (m, 4H), 2.59-2.49 (m, 4H), 2.39-2.27 (m, 7H), 1.63 (d, J = 6.7, 3H). LC-MS (Method B): R_{T} = 3.12 m/z = 442.8 [M − H]⁻. |
| 57 (Example 123) | 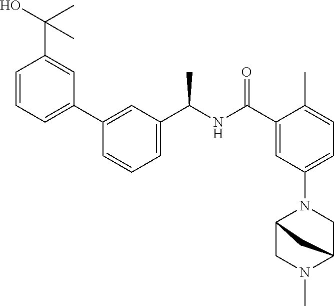 [SMILES: Cc1ccc(N2C[C@H]3C[C@@H]2CN3C)cc1C(=O)N[C@H](C)c1cccc(-c2cccc(C(C)(C)O)c2)c1] | N-[(1R)-1-[3-[3-(1-Hydroxy-1-methyl- ethyl)phenyl]phenyl]ethyl]-2-methyl-5- [(1R,4R)-5-methyl-2,5- diazabicyclo[2.2.1]heptan-2- yl]benzamide ¹H NMR (500 MHz, DMSO-d₆) δ 8.70 (d, J = 8.0, 1H), 7.75 (m, 1H), 7.69 (br s, 1H), 7.51 (br d, J = 7.5, 1H), 7.48-7.46 (m, 2H), 7.43 (t, J = 7.5, 1H), 7.41-7.37 (m, 2H), 6.99 (d, J = 8.5, 1H), 6.54 (dd, J = 8.0, 2.5, 1H), 6.48 (d, J = 2.5, 1H), 5.18 (quin, J = 7.0, 1H), 5.09 (s, 1H), 4.22 (br s, 1H), 3.38 (br s, 1H), 3.28 (dd, J = 9.0, 2.0, 1H), 3.13 (d, J = 9.0, 1H), 2.70 (dd, J = 9.5, 2.0, 1H), 2.44 (d, J = 9.5, 1H), 2.23 (s, 3H), 2.18 (s, 3H), 1.83 (br d, J = 9.0, 1H), 1.72 (br d, J = 9.0, 1H), 1.46 (m, 9H). LC-MS (Method B): R_{T} = 3.77, m/z = 483.0 [M − H]⁻. |
| 58 (Example 22) | 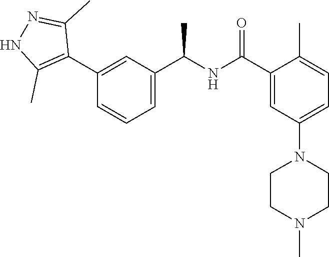 [SMILES: Cc1ccc(N2CCN(C)CC2)cc1C(=O)N[C@H](C)c1cccc(-c2c(C)n[nH]c2C)c1] | N-[(1R)-1-[3-(3,5-Dimethyl-1H-pyrazol- 4-yl)phenyl]ethyl]-2-methyl-5-(4- methylpiperazin-1-yl)benzamide ¹H NMR (500 MHz, CDCl₃) δ 7.45-7.35 (m, 1H), 7.31-7.27 (m, 2H), 7.19 (d, J = 7.6, 1H), 7.08 (d, J = 8.2, 1H), 6.93 (d, J = 2.4, 1H), 6.91-6.84 (m, 1H), 6.01 (br d, J = 7.9, 1H), 5.43-5.29 (m, 1H), 3.20-3.11 (m, 4H), 2.63-2.48 (m, 4H), 2.34 (s, 3H), 2.31 (s, 3H), 2.28 (s, 6H), 1.62 (d, J = 6.7, 3H). LC- MS (Method B): R_{T} = 3.04, m/z = 430.8 [M − H]⁻. |
| 59 (Example 22) | 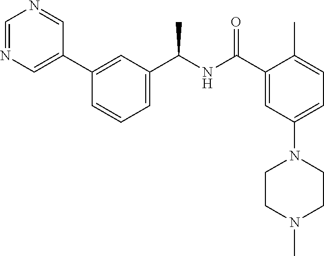 [SMILES: Cc1ccc(N2CCN(C)CC2)cc1C(=O)N[C@H](C)c1cccc(-c2cncnc2)c1] | 2-Methyl-5-(4-methylpiperazin-1-yl)-N- [(1R)-1-(3-pyrimidin-5- ylphenyl)ethyl]benzamide ¹H NMR (500 MHz, DMSO-d₆) δ 9.21 (br s, 1H), 9.15 (br s, 2H), 8.68 (d, J = 8.0, 1H), 7.85 (br s, 1H), 7.70 (dt, J = 6.5, 2.0, 1H), 7.54-7.50 (m, 2H), 7.07 (d, J = 8.5, 1H), 6.91 (dd, J = 8.5, 2.5, 1H), 6.87 (d, J = 2.5, 1H), 5.21 (quin, J = 7.0, 1H), 3.10-3.09 (m, 4H), 2.44-2.43 (m, 4H), 2.22 (s, 3H), 2.17 (s, 3H), 1.49 (d, J = 7.0, 3H). LC-MS (Method B): R_{T} = 3.07, m/z = 414.8 [M − H]⁻. |
| 60 (Example 22) | 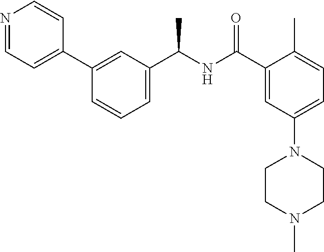 [SMILES: Cc1ccc(N2CCN(C)CC2)cc1C(=O)N[C@H](C)c1cccc(-c2ccncc2)c1] | 2-Methyl-5-(4-methylpiperazin-1-yl)-N- [(1R)-1-[3-(3- pyridyl)phenyl]ethyl]benzamide ¹H NMR (500 MHz, DMSO-d₆) δ 8.71 (d, J = 8.0, 1H), 8.65-8.64 (m, 2H), 7.85 (br s, 1H), 7.71-7.70 (m, 2H), 7.68-7.67 (m, 1H), 7.51-7.50 (m, 2H), 7.07 (d, J = 8.5, 1H), 6.91 (dd, J = 8.5, 2.5, 1H), 6.87 (d, J = 2.5, 1H), 5.21 (quin, J = 7.0, 1H), 3.10-3.09 (m, 4H), 2.44-2.43 (m, 4H), 2.22 (s, 3H), 2.17 (s, 3H), 1.48 (d, J = 7.0, 3H). LC-MS (Method B): R_{T} = 3.23, m/z = 413.9 [M − H]⁻. |
| 61 (Example 22) | 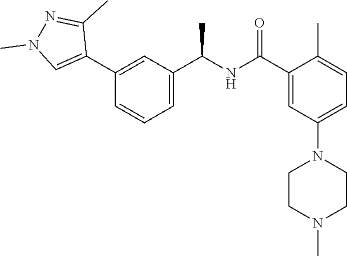 [SMILES: Cc1ccc(N2CCN(C)CC2)cc1C(=O)N[C@H](C)c1cccc(-c2cn(C)nc2C)c1] | N-[(1R)-1-[3-(1,3-Dimethylpyrazol-4- yl)phenyl]ethyl]-2-methyl-5-(4- methylpiperazin-1-yl)benzamide ¹H NMR (500 MHz, DMSO-d₆) δ 8.66 (d, J = 8.0, 1H), 7.85 (s, 1H), 7.45 (br s, 1H), 7.34 (t, J = 7.5, 1H), 7.28 (br d, J = 7.5, 1H), 7.24 (br d, J = 7.5, 1H), 7.06 (d, J = 8.0, 1H), 6.91 (dd, J = 8.5, 2.5, 1H), 6.85 (d, J = 2.5, 1H), 5.13 (quin, J = 7.0, 1H), 3.79 (s, 3H), 3.09-3.08 (m, 4H), 2.44-2.43 (m, 4H), 2.30 (s, 3H), 2.22 (s, 3H), 2.18 (s, 3H), 1.45 (d, J = 7.0, 3H). LC-MS (Method B): R_{T} = 3.38, m/z = 430.9 [M − H]⁻. |
| 62 (Example 22) | 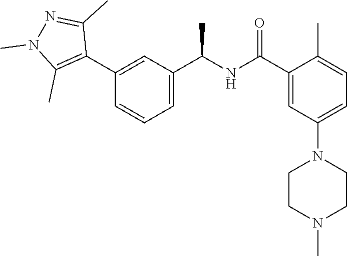 [SMILES: Cc1ccc(N2CCN(C)CC2)cc1C(=O)N[C@H](C)c1cccc(-c2c(C)nn(C)c2C)c1] | 2-Methyl-5-(4-methylpiperazin-1-yl)-N- [(1R)-1-[3-(1,3,5-trimethylpyrazol-4- yl)phenyl]ethyl]benzamide ¹H NMR (500 MHz, DMSO-d₆) δ 8.67 (d, J = 8.0, 1H), 7.37 (t, J = 7.5, 1H), 7.30- 7.27 (m, 2H), 7.12 (d, J = 7.5, 1H), 7.06 (d, J = 8.5, 1H), 6.90 (dd, J = 8.5, 2.5, 1H), 6.84 (d, J = 2.5, 1H), 5.15 (quin, J = 7.5, 1H), 3.70 (s, 3H), 3.09-3.08 (m, 4H), 2.44- 2.42 (m, 4H), 2.23 (s, 6H), 2.17 (s, 3H), 2.14 (s, 3H), 1.46 (d, J = 7.0, 3H). LC-MS (Method B): R_{T} = 3.53, m/z = 445.0 [M − H]⁻. |
| 63 (Example 22) | 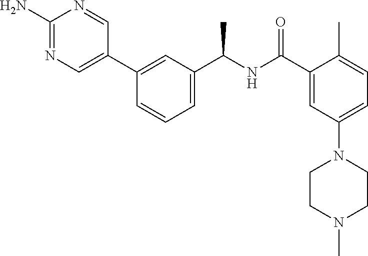 [SMILES: Cc1ccc(N2CCN(C)CC2)cc1C(=O)N[C@H](C)c1cccc(-c2cnc(N)nc2)c1] | N-[(1R)-1-[3-(2-Aminopyrimidin-5- yl)phenyl]ethyl]-2-methyl-5-(4- methylpiperazin-1-yl)benzamide ¹H NMR (500 MHz, DMSO-d₆) δ 8.66 (d, J = 8.5, 1H), 8.57 (s, 2H), 7.66 (br s, 1H), 7.49 (d, J = 7.5, 1H), 7.40 (t, J = 7.5, 1H), 7.34 (d, J = 7.5, 1H), 7.06 (d, J = 8.5, 1H), 6.91 (dd, J = 8.0, 2.5, 1H), 6.85 (d, J = 2.5, 1H), 6.79 (br s, 2H), 5.17 (quin, J = 7.0, 1H), 3.09-3.08 (m, 4H), 2.45- 2.44 (m, 4H), 2.23 (s, 3H), 2.17 (s, 3H), 1.46 (d, J = 7.5, 3H). LC-MS (Method B): R_{T} = 3.21, m/z = 429.9 [M − H]⁻. |
| 64 (Example 22) | 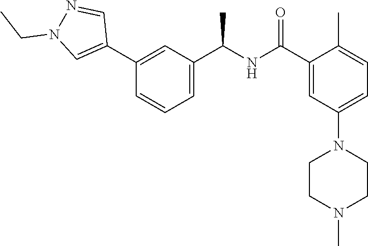 [SMILES: CCn1cc(-c2cccc([C@@H](C)NC(=O)c3cc(N4CCN(C)CC4)ccc3C)c2)cn1] | N-[(1R)-1-[3-(1-Ethylpyrazol-4- yl)phenyl]ethyl]-2-methyl-5-(4- methylpiperazin-1-yl)benzamide ¹H NMR (500 MHz, DMSO-d₆) δ 8.63 (d, J = 8.0, 1H), 8.15 (s, 1H), 7.83 (s, 1H), 7.61 (br s, 1H), 7.43 (d, J = 7.5, 1H), 7.32 (t, J = 7.5, 1H), 7.21 (d, J = 7.5, 1H), 7.06 (d, J = 8.0, 1H), 6.91 (dd, J = 8.5, 2.5, 1H), 6.86 (d, J = 2.5, 1H), 5.12 (quin, J = 7.0, 1H), 4.16 (q, J = 7.5, 2H), 3.10-3.09 (m, 4H), 2.44-2.43 (m, 4H), 2.22 (s, 3H), 2.18 (s, 3H), 1.45 (d, J = 7.0, 3H), 1.41 (t, J = 7.5, 3H). LC-MS (Method B): R_{T} = 3.42, m/z = 431.0 [M − H]⁻. |
| 65 (Example 22) | 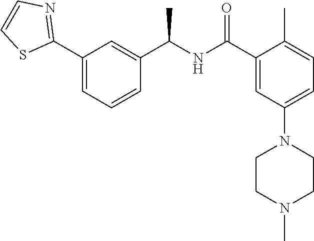 [SMILES: Cc1ccc(N2CCN(C)CC2)cc1C(=O)N[C@H](C)c1cccc(-c2nccs2)c1] | 2-Methyl-5-(4-methylpiperazin-1-yl)-N- [(1R)-1-(3-thiazol-2- ylphenyl)ethyl]benzamide ¹H NMR (500 MHz, CDCl₃) δ 8.02 (s, 1H), 7.91-7.81 (m, 2H), 7.51-7.39 (m, 2H), 7.34 (d, J = 3.4, 1H), 7.08 (d, J = 8.2, 1H), 6.95 (d, J = 2.4, 1H), 6.93-6.82 (m, 1H), 6.02 (br d, J = 7.6, 1H), 5.38 (quin, J = 7.2, 1H), 3.22- 3.13 (m, 4H), 2.61-2.53 (m, 4H), 2.35 (s, 3H), 2.32 (s, 3H), 1.64 (d, J = 7.0, 3H). LC- MS (Method B): R_{T} = 3.40 m/z = 419.7 [M − H]⁻. |
| 66 (Example 22) | 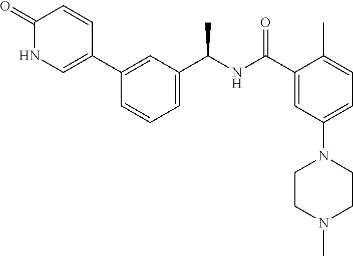 [SMILES: Cc1ccc(N2CCN(C)CC2)cc1C(=O)N[C@H](C)c1cccc(-c2ccc(=O)[nH]c2)c1] | 2-Methyl-5-(4-methylpiperazin-1-yl)-N- [(1R)-1-[3-(6-oxo-1,6-dihydropyridin-3- yl)phenyl]ethyl]benzamide ¹H NMR (500 MHz, DMSO-d₆) δ 11.85 (br s, 1H), 8.64 (d, J = 8.4, 1H), 7.83 (dd, J = 2.8, 9.5, 1H), 7.69 (d, J = 2.3, 1H), 7.60 (s, 1H), 7.42 (d, J = 7.6, 1H), 7.37 (t, J = 7.6, 1H), 7.30 (d, J = 7.6, 1H), 7.06 (d, J = 8.4, 1H), 6.91 (dd, J = 2.8, 8.4, 1H), 6.84 (d, J = 2.6, 1H), 6.45 (d, J = 9.5, 1H), 5.15 (quin, J = 7.3, 1H), 3.10 (br s, 4H), 2.46-2.42 (m, 4H), 2.22 (s, 3H), 2.16 (s, 3H), 1.45 (d, J = 7.0, 3H). LC-MS (Method B): R_{T} = 3.02, m/z = 431.7 [M + H]⁺. |
| 67 (Example 22) | 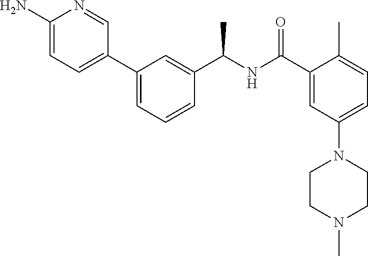 [SMILES: Cc1ccc(N2CCN(C)CC2)cc1C(=O)N[C@H](C)c1cccc(-c2ccc(N)nc2)c1] | N-[(1R)-1-[3-(6-Amino-3- pyridyl)phenyl]ethyl]-2-methyl-5-(4- methylpiperazin-1-yl)benzamide ¹H NMR (500 MHz, DMSO-d₆) δ 8.67 (d, J = 8.5, 1H), 8.25 (d, J = 2.3, 1H), 7.68 (s, 1H), 7.62 (s, 1H), 7.42 (s, 1H), 7.39-7.33 (m, 1H), 7.29 (s, 1H), 7.06 (d, J = 8.4, 1H), 6.91 (dd, J = 2.4, 8.5, 1H), 6.85 (d, J = 2.0, 1H), 6.53 (d, J = 8.5, 1H), 6.06 (s, 2H), 5.20-5.11 (m, 1H), 3.13-3.06 (m, 4H), 2.44 (br s, 4H), 2.23 (s, 3H), 2.18 (s, 3H), 1.46 (d, J = 7.0, 3H). LC-MS (Method B): RT = 3.15, m/z = 428.9 [M − H]⁻. |

### Example 68: N-[(1R)-1-[3-[3-[(Dimethylamino)methyl]phenyl]phenyl]ethyl]-2-methyl-5-(4-methylpiperazin-1-yl)benzamide Dihydrochloride Salt

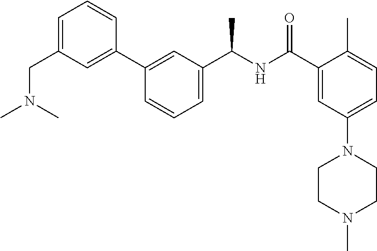 [SMILES: Cc1ccc(N2CCN(C)CC2)cc1C(=O)N[C@H](C)c1cccc(-c2cccc(CN(C)C)c2)c1]

N-[(1R)-1-[3-[3-(Chloromethyl)phenyl]phenyl]ethyl]-2-methyl-5-(4-methylpiperazin-1-yl)benzamide (90 mg, 195 μmol)—prepared in a similar manner to N-[(1R)-1-[3-(4-methoxyphenyl)phenyl]ethyl]-2-methyl-5-(4-methylpiperazin-1-yl)benzamide (Example 40)—was dissolved in DMF (2 mL) to this was added dimethylamine (2 M in THF, 487 μL) and the mixture was stirred at RT for 3 hours. Diethyl ether (70 mL) and water (70 mL) were added, and the phases separated. The organic phase was washed with brine (60 mL), dried (Na₂SO₄) and the solvent removed in vacuo. Purification by FCC (eluting with 0-100% MeOH in DCM followed by 1N NH3 in MeOH) gave the product as an oil that would not crystallise. DCM (2 mL) and 4N HCl in 1,4-dioxane (0.8 mL) were added and the solvent removed almost to dryness in vacuo then diethyl ether was added to give a cloudy precipitate. The solvent was removed in vacuo and gave N-[(1R)-1-[3-[3-[(dimethylamino)methyl]phenyl]phenyl]ethyl]-2-methyl-5-(4-methylpiperazin-1-yl)benzamide dihydrochloride salt (10 mg, 9%) as a white crystalline solid. ¹H NMR (500 MHz, DMSO-d₆) δ 10.75 (br s, 1H), 10.61 (br s, 1H), 8.75 (d, J=8.0, 1H), 7.93 (br s, 1H), 7.76 (m, 2H), 7.61-7.53 (m, 3H), 7.48 (t, J=7.5, 1H), 7.43 (br d, J=8.0, 1H), 7.12 (d, J=8.5, 1H), 6.99 (dd, J=8.5, 3.0, 1H), 6.94 (d, J=3.0, 1H), 5.19 (quin, J=7.0, 1H), 4.35 (d, J=5.0, 2H), 3.81-3.80 (m, 2H), 3.41-3.40 (m, 2H), 3.12-3.11 (m, 2H), 3.05-3.04 (m, 2H), 2.82 (d, J=5.0, 3H), 2.74 (d, J=5.0, 6H), 2.19 (s, 3H), 1.49 (d, J=7.0, 3H). LC-MS (Method B): R_{T}=3.61, m/z=469.9 [M−H]⁻.

### Example 69: N-[(1R)-1-[3-(1,2-Dihydroxyethyl)phenyl]ethyl]-2-methyl-5-(4-methylpiperazin-1-yl)benzamide

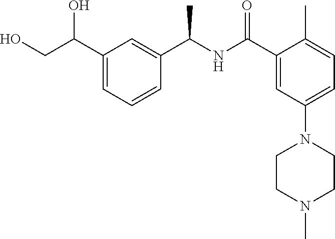 [SMILES: Cc1ccc(N2CCN(C)CC2)cc1C(=O)N[C@H](C)c1cccc(C(O)CO)c1]

Osmium tetroxide (2 crystals, cat.) was added to a solution of 2-methyl-5-(4-methylpiperazin-1-yl)-N-[(1R)-1-(3-vinylphenyl)ethyl]benzamide (305 mg, 839 μmol)—prepared in a similar manner to N-[(1R)-1-[3-(4-methoxyphenyl)phenyl]ethyl]-2-methyl-5-(4-methylpiperazin-1-yl)benzamide (Example 37)—and 4-methylmorpholine N-oxide (197 mg, 1.68 mmol) in THF (10 mL) and water (10 mL) and the reaction mixture was allowed to stir at RT for 4 hours. Water (50 mL), a saturated aqueous solution of sodium thiosulfate (50 mL) and diethyl ether (80 mL) were added, and the phases separated. The aqueous phase was extracted with diethyl ether (70 mL) and the combined organic phases washed with a saturated aqueous solution of sodium thiosulfate (100 mL), dried (Na₂SO₄) and the solvent removed in vacuo. Purification by FCC (eluting with 0-70% MeOH in DCM) gave N-[(1R)-1-[3-(1,2-dihydroxyethyl)phenyl]ethyl]-2-methyl-5-(4-methylpiperazin-1-yl)benzamide (90 mg, 26%) as a white crystalline solid. ¹H NMR (500 MHz, DMSO-d₆) δ 8.64 (dd, J=8.0, 4.5, 1H), 7.39 (m, 1H), 7.29-7.24 (m, 2H), 7.19-7.17 (m, 1H), 7.06 (d, J=8.0, 1H), 6.90 (dd, J=8.5, 2.5, 1H), 6.85 (d, J=2.5, 1H), 5.22 (t, J=4.0, 1H), 5.10 (quin, J=7.0, 1H), 4.70 (t, J=6.0, 1H), 4.53 (q, J=5.0, 1H), 3.42-3.40 (m, 2H), 3.11-3.09 (m, 4H), 2.45-2.43 (m, 4H), 2.22 (s, 3H), 2.18 (s, 3H), 1.42 (d, J=7.0, 3H). LC-MS (Method B): R_{T}=2.62, m/z=396.8 [M−H]⁻.

### Further Examples

The following examples were prepared in a similar manner to N-[(1R)-1-[3-(1,2-dihydroxyethyl)phenyl]ethyl]-2-methyl-5-(4-methylpiperazin-1-yl)benzamide (Example 69), from the intermediate stated, which were each in turn prepared in a similar manner to N-[(1R)-1-[3-(4-methoxyphenyl)phenyl]ethyl]-2-methyl-5-(4-methylpiperazin-1-yl)benzamide (Example 40).

| Example | | |
|---|---|---|
| (Intermediate) | Structure | Name and Analytical Data |
| 70 (2-Methyl-5- (4-methyl- piperazin- 1-yl)-N- [(1R)-1-[3-(4- vinylphenyl) phenyl]ethyl] benzamide) | 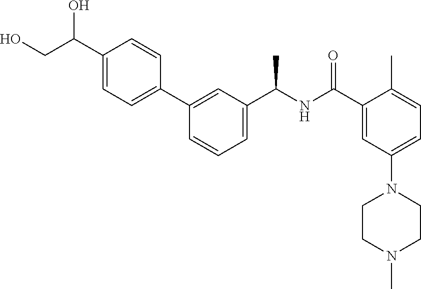 [SMILES: Cc1ccc(N2CCN(C)CC2)cc1C(=O)N[C@H](C)c1cccc(-c2ccc(C(O)CO)cc2)c1] | N-[(1R)-1-[3-[4-(1,2- Dihydroxyethyl)phenyl]phenyl]ethyl]- 2-methyl-5-(4-methyl piperazin- 1-yl)benzamide ¹H NMR (500 MHz, DMSO-d₆) δ 8.70 (d, J = 8.0, 1H), 7.70 (br s, 1H), 7.61 (d, J = 8.0, 2H), 7.52 (br d, J = 7.5, 1H), 7.43 (d, J = 8.5, 2H), 7.41 (m, 1H), 7.36 (br d, J = 7.5, 1H), 7.07 (d, J = 8.5, 1H), 6.91 (dd, J = 8.5, 3.0, 1H), 6.87 (d, J = 2.0, 1H), 5.27 (d, J = 4.5, 1H), 5.18 (quin, J = 7.0, 1H), 4.74 (t, J = 5.5, 1H), 4.59-4.57 (m, 1H), 3.47 (t, J = 5.5, 2H), 3.09-3.07 (m, 4H), 2.44-2.41 (m, 4H), 2.22 (s, 3H), 2.18 (s, 3H), 1.47 (d, J = 7.0, 3H). LC-MS (Method B): R_{T} = 2.86, m/z = 472.9 [M − H]⁻. |
| 71 (2-Methyl-5- (4-methyl- piperazin- 1-yl)-N- [(1R)-1-[3-(3- vinylphenyl) phenyl]ethyl] benzamide) | 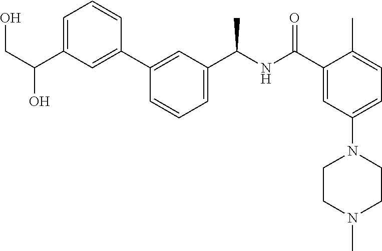 [SMILES: Cc1ccc(N2CCN(C)CC2)cc1C(=O)N[C@H](C)c1cccc(-c2cccc(C(O)CO)c2)c1] | N-[(1R)-1-[3-[3-(1,2- Dihydroxyethyl)phenyl]phenyl]ethyl]- 2-methyl-5-(4-methylpiperazin- 1-yl)benzamide ¹H NMR (500 MHz, DMSO-d₆) δ 8.72 (d, J = 8.0, 1H), 7.69 (m, 1H), 7.63 (m, 1H), 7.52 (m, 2H), 7.45-7.34 (m, 4H), 7.07 (d, J = 8.5, 1H), 6.91 (dd, J = 8.5, 2.5, 1H), 6.86 (t, J = 2.5, 1H), 5.31 (br s, 1H), 5.19 (quin, J = 7.0, 1H), 4.77 (br s, 1H), 4.62-4.60 (m, 1H), 3.49 (br d, J = 5.5, 1H), 3.10-3.08 (m, 4H), 2.44- 2.42 (m, 4H), 2.22 (s, 3H), 2.19 (s, 3H), 1.48 (d, J = 7.0, 3H). LC-MS (Method B): R_{T} = 2.90, m/z = 472.8 [M − H]⁻. |

### Example 72: 2-Methyl-N-[(1R)-1-[3-(1-methylpyrazol-4-yl)phenyl]ethyl]-5-piperazin-1-yl-benzamide

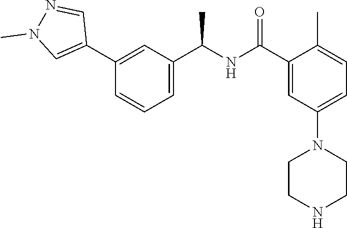 [SMILES: Cc1ccc(N2CCNCC2)cc1C(=O)N[C@H](C)c1cccc(-c2cnn(C)c2)c1]

Using General Procedure 4 with tert-butyl 4-[4-methyl-3-[[(1R)-1-[3-(1-methylpyrazol-4-yl)phenyl]ethyl]carbamoyl]phenyl]piperazine-1-carboxylate (503 mg, 999 μmol)—prepared in a similar manner to N-[(1R)-1-[3-(4-methoxyphenyl)phenyl]ethyl]-2-methyl-5-(4-methylpiperazin-1-yl)benzamide (Example 40)—gave 2-methyl-N-[(1R)-1-[3-(1-methylpyrazol-4-yl)phenyl]ethyl]-5-piperazin-1-yl-benzamide (338 mg, 80%) as a white crystalline solid. ¹H NMR (500 MHz, DMSO-d₆) δ 8.63 (d, J=8.0, 1H), 8.10 (s, 1H), 7.82 (s, 1H), 7.60 (br s, 1H), 7.42 (br d, J=7.5, 1H), 7.32 (t, J=7.5, 1H), 7.21 (br d, J=7.5, 1H), 7.06 (d, J=8.5, 1H), 6.89 (dd, J=8.5, 2.5, 1H), 6.84 (d, J=2.5, 1H), 5.12 (quin, J=7.0, 1H), 3.87 (s, 3H), 3.03-3.01 (m, 4H), 2.82-2.80 (m, 4H), 2.18 (s, 3H), 1.45 (d, J=7.0, 3H). LC-MS (Method B): R_{T}=3.25, m/z=402.8 [M−H]⁻.

### Further Examples

The following examples were prepared in a similar manner to 2-methyl-N-[(1R)-1-[3-(1-methylpyrazol-4-yl)phenyl]ethyl]-5-piperazin-1-yl-benzamide (Example 72), from the intermediate stated, which were each in turn prepared in a similar manner to N-[(1R)-1-[3-(4-methoxyphenyl)phenyl]ethyl]-2-methyl-5-(4-methylpiperazin-1-yl)benzamide (Example 40).

| Example | Structure | Name and Analytical Data |
|---|---|---|
| 73 | 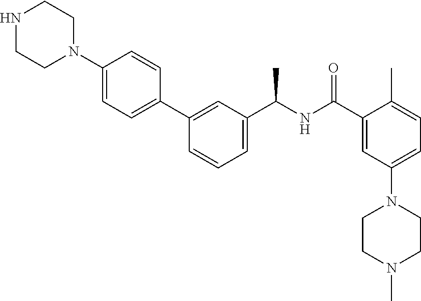 [SMILES: Cc1ccc(N2CCN(C)CC2)cc1C(=O)N[C@H](C)c1cccc(-c2ccc(N3CCNCC3)cc2)c1] | 2-Methyl-5-(4-methylpiperazin-1-yl)-N- [(1R)-1-[3-(4-piperazin-1- ylphenyl)phenyl]ethyl]benzamide dihydrochloride salt ¹H NMR (500 MHz, DMSO-d₆) δ 11.07 (br s, 1H), 9.26 (br s, 2H), 8.74 (d, J = 8.0, 1H), 7.67 (br s, 1H), 7.59 (d, J = 8.5, 2H), 7.49 (d, J = 8.0, 1H), 7.39 (t, J = 7.5, 1H), 7.31 (d, J = 7.5, 1H), 7.13-7.10 (m, 3H), 6.99 (dd, J = 8.5, 2.5, 1H), 6.93 (d, J = 2.5, 1H), 5.18 (quin, J = 7.5, 1H), 3.78 (m, 2H), 3.46-3.44 (m, 6H), 3.23-3.21 (m, 4H), 3.12-3.10 (m, 4H), 2.80 (d, J = 5.0, 3H), 2.20 (s, 3H), 1.47 (d, J = 7.0, 3H). LC-MS (Method B): R_{T} = 3.55, m/z = 496.9 [M − H]⁻. |
| 74 | 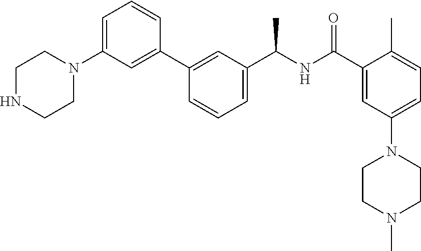 [SMILES: Cc1ccc(N2CCN(C)CC2)cc1C(=O)N[C@H](C)c1cccc(-c2cccc(N3CCNCC3)c2)c1] | 2-Methyl-5-(4-methylpiperazin-1-yl)-N- [(1R)-1-[3-(3-piperazin-1-ylphenyl)phenyl] ethyl]benzamide ¹H NMR (500 MHz, DMSO-d₆) δ 8.70 (d, J = 8.0, 1H), 7.67 (br s, 1H), 7.50 (d, J = 7.5, 1H), 7.40-7.38 (m, 1H), 7.36-7.34 (m, 1H), 7.29 (t, J = 8.0, 1H), 7.12 (br s, 1H), 7.06 (d, J = 8.5, 1H), 7.03 (d, J = 7.5, 1H), 6.92-6.90 (m, 2H), 6.85 (d, J = 2.5, 1H), 5.18 (quin, J = 7.0, 1H), 3.10-3.08 (m, 8H), 2.85-2.83 (m, 4H), 2.43-2.41 (m, 4H), 2.22 (s, 3H), 2.18 (s, 3H), 1.47 (d, J = 7.0, 3H). LC-MS (Method B): R_{T} = 3.32, m/z = 496.9 [M − H]⁻. |

### Example 75: 2-Methyl-N-[(1R)-1-[3-[4-[methyl-[2-(methylamino)ethyl]carbamoyl]phenyl]phenyl] ethyl]-5-(4-methylpiperazin-1-yl)benzamide

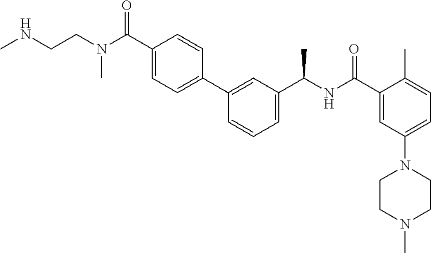 [SMILES: CNCCN(C)C(=O)c1ccc(-c2cccc([C@@H](C)NC(=O)c3cc(N4CCN(C)CC4)ccc3C)c2)cc1]

Using General Procedure 5 with palladium hydroxide, 20% on carbon (30 mg) and benzyl N-methyl-N-[2-[methyl-[4-[3-[(1R)-1-[[2-methyl-5-(4-methylpiperazin-1-yl)benzoyl]amino]ethyl]phenyl]benzoyl]amino]ethyl]carbamate (109 mg, 165 μmol)—prepared in a similar manner to N-[(1R)-1-[3-(4-methoxyphenyl)phenyl]ethyl]-2-methyl-5-(4-methylpiperazin-1-yl)benzamide (Example 40)—gave 2-methyl-N-[(1R)-1-[3-[4-[methyl-[2-(methylamino)ethyl]carbamoyl]phenyl]phenyl]ethyl]-5-(4-methylpiperazin-1-yl)benzamide (50 mg, 55%) as a white solid. ¹H NMR (500 MHz, DMSO-d₆) δ 8.71 (d, J=8.0, 1H), 7.76 (br s, 1H), 7.72 (d, J=8.0, 2H), 7.58 (d, J=7.5, 1H), 7.50 (d, J=7.5, 2H), 7.46-7.44 (m, 1H), 7.41 (d, J=8.0, 1H), 7.06 (d, J=8.5, 1H), 6.91 (dd, J=8.0, 2.5, 1H), 6.86 (d, J=2.5, 1H), 5.20 (quin, J=7.5, 1H), 3.53 (br s, 1H), 3.09-3.07 (m, 4H), 2.98 (br s, 3H), 2.71 (br s, 1H), 2.60 (br s, 1H), 2.43-2.41 (m, 4H), 2.36 (br s, 1H), 2.22 (s, 3H), 2.18 (s, 3H), 2.14 (br s, 1H), 1.48 (d, J=7.0, 3H). LC-MS (Method B): R_{T}=3.45, m/z=527.0 [M−H]⁻.

### Further Examples

The following examples were prepared in a similar manner to N-[(1R)-1-[3-(1,2-dihydroxyethyl)phenyl]ethyl]-2-methyl-5-(4-methylpiperazin-1-yl)benzamide (Example 75), from the intermediate stated, which were each in turn prepared in a similar manner to N-[(1R)-1-[3-(4-methoxyphenyl)phenyl]ethyl]-2-methyl-5-(4-methylpiperazin-1-yl)benzamide (Example 40)

| Example | | |
|---|---|---|
| (Intermediate) | Structure | Name and Analytical Data |
| 76 (N-[(1R)-1-[3- (6-benzyloxy- 2- pyridyl)phenyl] ethyl]-2- methyl-5-(4- methyl- piperazin-1- yl)benzamide) | 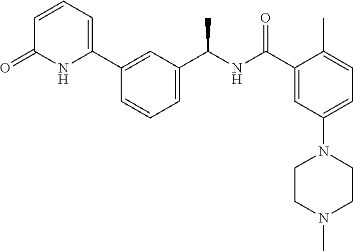 [SMILES: Cc1ccc(N2CCN(C)CC2)cc1C(=O)N[C@H](C)c1cccc(-c2cccc(=O)[nH]2)c1] | 2-Methyl-5-(4-methylpiperazin-1-yl)- N-[(1R)-1-[3-(6-oxo-1H-pyridin-2- yl)phenyl]ethyl]benzamide ¹H NMR (500 MHz, DMSO-d₆) δ 11.84 (br s, 1H), 8.64 (d, J = 8.2, 1H), 7.83 (dd, J = 2.7, 9.6, 1H), 7.71-7.67 (m, 1H), 7.60 (s, 1H), 7.44-7.41 (m, 1H), 7.37 (t, J = 7.6, 1H), 7.32-7.29 (m, 1H), 7.06 (d, J = 8.4, 1H), 6.91 (dd, J = 2.6, 8.2, 1H), 6.84 (d, J = 2.6, 1H), 6.45 (d, J = 9.5, 1H), 5.15 (quin, J = 7.2, 1H), 3.13-3.07 (m, 4H), 2.45-2.42 (m, 4H), 2.22 (s, 3H), 2.17 (s, 3H), 1.45 (d, J = 7.2, 3H). LC-MS (Method B): R_{T} = 3.02, m/z = 431.8 [M + H]⁺. |
| 77 (benzyl 4-[4- [3-[(1R)-1-[2- methyl-5-(4- methyl- piperazin-1- yl)benzoyl] amino]ethyl] phenyl]phenyl] piperidine-1- carboxylate) | 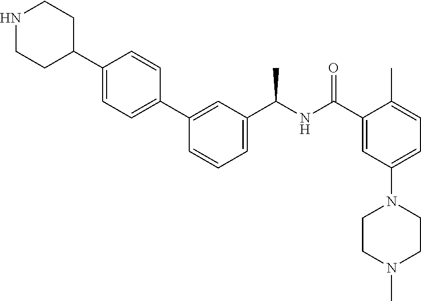 [SMILES: Cc1ccc(N2CCN(C)CC2)cc1C(=O)N[C@H](C)c1cccc(-c2ccc(C3CCNCC3)cc2)c1] | 2-Methyl-5-(4-methylpiperazin-1-yl)- N-[(1R)-1-[3-[4-(4- piperidyl)phenyl]phenyl]ethyl]benzamide ¹H NMR (500 MHz, CDCl₃) δ 7.58 (s, 1H), 7.55-7.47 (m, 3H), 7.42 (t, J = 7.6, 1H), 7.34 (d, J = 7.6, 1H), 7.30 (d, J = 7.9, 2H), 7.08 (d, J = 8.5, 1H), 6.94 (d, J = 2.7, 1H), 6.87 (dd, J = 2.7, 8.5, 1H), 5.97 (br d, J = 8.2, 1H), 5.39 (quin, J = 7.2, 1H), 3.23 (br d, J = 11.9, 2H), 3.19-3.12 (m, 4H), 2.78 (dt, J = 2.4, 12.2, 2H), 2.73-2.62 (m, 1H), 2.59-2.52 (m, 4H), 2.33-2.31 (m, 6H), 1.88 (br d, J = 13.4, 2H), 1.75-1.65 (m, 2H), 1.65-1.61 (m, 3H). LC-MS (Method B): R_{T} = 5.84, m/z = 496.0 [M − H]⁺. |

### Example 78: N-[(1R)-1-[3-[5-[(Cyclopentylamino)methyl]-2-thienyl]phenyl]ethyl]-2-methyl-5-(4-methylpiperazin-1-yl)benzamide Dihydrochloride Salt

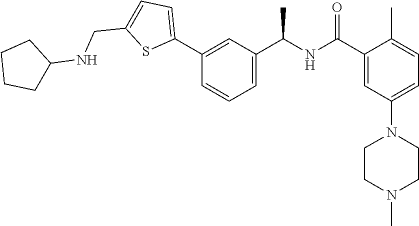 [SMILES: Cc1ccc(N2CCN(C)CC2)cc1C(=O)N[C@H](C)c1cccc(-c2ccc(CNC3CCCC3)s2)c1]

Acetic acid (50 μL, 874 μmol) was added to a solution of N-[(1R)-1-[3-(5-formyl-2-thienyl)phenyl]ethyl]-2-methyl-5-(4-methylpiperazin-1-yl)benzamide (82 mg, 183 μmol)—prepared in a similar manner to N-[(1R)-1-[3-(4-methoxyphenyl)phenyl]ethyl]-2-methyl-5-(4-methylpiperazin-1-yl)benzamide (Example 40)—and cyclopentylamine (27 μL, 275 μmol) in MeOH (15 mL) and the reaction mixture heated to 50° C. for 2 hours. The reaction mixture was allowed to cool to RT and sodium cyanoborohydride (35 mg, 550 μmol) was added and the reaction mixture allowed to stir at RT for a further 2 hours. The solvent was removed in vacuo and the material redissolved in MeOH (˜2 mL) and loaded directly on to a pre-equilibrated 2 g SCX cartridge and eluted with MeOH followed by 1N NH₃ in MeOH. The fractions that contained compound were collected and the solvent removed in vacuo. DCM (2 mL) and 4N HCl in 1,4-dioxane (0.8 mL) were added, then the solvent was removed in vacuo to give a black residue. Purification by FCC (eluting with 0-60% MeOH in DCM) gave N-[(1R)-1-[3-[5-[(cyclopentylamino)methyl]-2-thienyl]phenyl]ethyl]-2-methyl-5-(4-methylpiperazin-1-yl)benzamide dihydrochloride salt (40 mg, 35%) as a pale yellow solid. ¹H NMR (500 MHz, DMSO-d₆) δ 9.43 (br s, 1H), 8.77 (d, J=8.0, 1H), 7.68 (br s, 1H), 7.53 (m, 1H), 7.47 (d, J=3.5, 1H), 7.43-7.36 (m, 3H), 7.11 (d, J=8.5, 1H), 6.98 (dd, J=8.5, 2.5, 1H), 6.92 (d, J=2.5, 1H), 5.15 (quin, J=7.0, 1H), 4.37 (s, 2H), 3.48-3.46 (m, 2H), 3.17 (br s, 2H), 2.70 (br s, 2H), 2.19 (s, 3H), 1.99-1.97 (m, 2H), 1.73-1.71 (m, 4H), 1.64-1.62 (m, 2H), 1.47 (d, J=7.0, 3H). LC-MS (Method B): R_{T}=4.11, m/z=516.0 [M−H]⁻.

### Example 79: N-[(1R)-1-[3-[5-(Hydroxymethyl)-2-thienyl]phenyl]ethyl]-2-methyl-5-(4-methylpiperazin-1-yl)benzamide

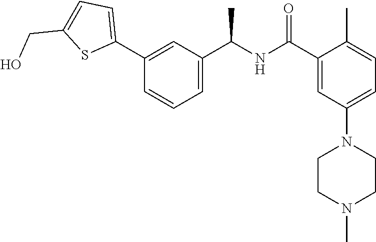 [SMILES: Cc1ccc(N2CCN(C)CC2)cc1C(=O)N[C@H](C)c1cccc(-c2ccc(CO)s2)c1]

Sodium borohydride (14 mg, 366 μmol) was added to a solution of N-[(1R)-[3-(5-formyl-2-thienyl)phenyl]ethyl]-2-methyl-5-(4-methylpiperazin-1-yl)benzamide (82 mg, 183 μmol)—prepared in a similar manner to N-[(1R)-1-[3-(4-methoxyphenyl)phenyl]ethyl]-2-methyl-5-(4-methylpiperazin-1-yl)benzamide (Example 40)—in MeOH (5 mL) and the reaction mixture allowed to stir at RT overnight. The solvent was removed in vacuo and purification by FCC (eluting with 0-30% MeOH in DCM) gave N-[(1R)-1-[3-[5-(hydroxymethyl)-2-thienyl]phenyl]ethyl]-2-methyl-5-(4-methylpiperazin-1-yl)benzamide (55 mg, 63%) as a pale yellow solid. ¹H NMR (500 MHz, DMSO-d₆) δ 8.71 (d, J=8.0, 1H), 7.65 (br s, 1H), 7.50 (m, 1H), 7.37 (t, J=7.5, 1H), 7.33 (d, J=3.5, 1H), 7.30 (br d, J=8.0, 1H), 7.07 (d, J=8.5, 1H), 6.79 (d, J=3.5, 1H), 6.92 (dd, J=8.5, 2.5, 1H), 6.88 (d, J=2.5, 1H), 5.52 (br s, 1H), 5.13 (quin, J=7.0, 1H), 4.64 (br s, 2H), 3.12-3.10 (m, 4H), 2.45-2.43 (m, 4H), 2.22 (s, 3H), 2.18 (s, 3H), 1.45 (d, J=7.0, 3H). LC-MS (Method B): R_{T}=3.18, m/z=448.8 [M−H]⁻.

### Further Examples

The following examples were prepared in a similar manner to N-[(1R)-1-[3-[5-(hydroxymethyl)-2-thienyl]phenyl]ethyl]-2-methyl-5-(4-methylpiperazin-1-yl)benzamide (Example 79).

| Example | Structure | Name and Analytical Data |
|---|---|---|
| 80 | 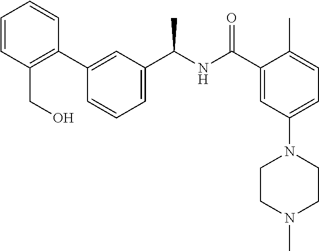 [SMILES: Cc1ccc(N2CCN(C)CC2)cc1C(=O)N[C@H](C)c1cccc(-c2ccccc2CO)c1] | N-[(1R)-1-[3-[2- (Hydroxymethyl)phenyl]phenyl]ethyl]-2- methyl-5-(4-methylpiperazin-1-yl)benzamide ¹H NMR (500 MHz, DMSO-d₆) δ 8.66 (d, J = 8.0, 1H), 7.60 (d, J = 7.5, 1H), 7.42-7.37 (m, 4H), 7.32 (td, J = 7.5, 1.0, 1H), 7.25 (dt, J = 6.5, 2.0, 1H), 7.22 (dd, J = 7.5, 1.0, 1H), 7.05 (d, J = 8.0, 1H), 6.90 (dd, J = 8.5, 3.0, 1H), 6.84 (d, J = 3.0, 1H), 5.17 (quin, J = 7.0, 1H), 5.11 (t, J = 5.0, 1H), 4.42 (d, J = 5.0, 2H), 3.08-3.06 (m, 4H), 2.43-2.41 (m, 4H), 2.22 (s, 3H), 2.17 (s, 3H), 1.46 (d, J = 7.0, 3H). LC-MS (Method B): R_{T} = 3.35, m/z = 442.9 [M − H]⁻. |

### Example 81: N-[(1R)-1-[3-[3,5-Bis(hydroxymethyl)phenyl]phenyl]ethyl]-2-methyl-5-(4-methylpiperazin-1-yl)benzamide

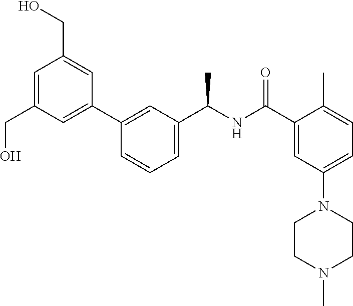 [SMILES: Cc1ccc(N2CCN(C)CC2)cc1C(=O)N[C@H](C)c1cccc(-c2cc(CO)cc(CO)c2)c1]

### Step A: tert-Butyl-[[3-[[tert-butyl(dimethyl)silyl]oxymethyl]-5-(4,4,5,5-tetramethyl-1,3,2-dioxaborolan-2-yl)phenyl]methoxy]-dimethyl-silane

1,1′-Bis(diphenylphosphino)ferrocenepalladium(II) dichloride (160 mg, 219 μmol) was added to a degassed solution of [3-bromo-5-[[tert-butyl(dimethyl)silyl]oxymethyl]phenyl]methoxy-tert-butyl-dimethyl-silane (976 mg, 2.19 mmol), bis(pinacolato)diboron (667 mg, 2.63 mmol) and potassium acetate (645 mg, 6.57 mmol) in 1,4-dioxane (15 mL) and the reaction mixture heated to 100° C. overnight. The reaction mixture was allowed to cool to RT and water (50 mL) and ethyl acetate (70 mL) added. The phases were separated, and the aqueous phase extracted with ethyl acetate (70 mL). The combined organic phases were washed with brine (100 mL), dried (Na₂SO₄) and the solvent removed in vacuo. Purification by FCC (eluting with 0-50% ethyl acetate in petroleum ether) gave tert-butyl-[[3-[[tert-butyl(dimethyl)silyl]oxymethyl]-5-(4,4,5,5-tetramethyl-1,3,2-dioxaborolan-2-yl)phenyl]methoxy]-dimethyl-silane (542 mg, 50%) as a colourless oil which solidified on standing to give a tan crystalline solid. ¹H NMR (500 MHz, DMSO-d₆) δ 7.47 (br s, 3H), 4.66 (br s, 4H), 1.25 (s, 12H), 0.85 (s, 18H), 0.00 (s, 12H).

### Step B: N-[(1R)-1-[3-[3,5-Bis[[tert-butyl(dimethyl)silyl]oxymethyl]phenyl]phenyl]ethyl]-2-methyl-5-(4-methylpiperazin-1-yl)benzamide

Using General Procedure 2 with N-[(1R)-1-(3-bromophenyl)ethyl]-2-methyl-5-(4-methylpiperazin-1-yl)benzamide (253 mg, 608 μmol) (Example 22) and tert-butyl-[[3-[[tert-butyl(dimethyl)silyl]oxymethyl]-5-(4,4,5,5-tetramethyl-1,3,2-dioxaborolan-2-yl)phenyl]methoxy]-dimethyl-silane (329 mg, 668 μmol) at 85° C. overnight gave N-[(1R)-1-[3-[3,5-bis[[tert-butyl(dimethyl)silyl]oxymethyl]phenyl]phenyl]ethyl]-2-methyl-5-(4-methylpiperazin-1-yl)benzamide (250 mg, 59%) as a white solid. The material was used directly in Step C.

### Step C: N-[(1R)-1-[3-[3,5-Bis(hydroxymethyl)phenyl]phenyl]ethyl]-2-methyl-5-(4-methylpiperazin-1-yl)benzamide

Using General Procedure 4 with N-[(1R)-1-[3-[3,5-bis[[tert-butyl(dimethyl)silyl]oxymethyl]phenyl]phenyl]ethyl]-2-methyl-5-(4-methylpiperazin-1-yl)benzamide (250 mg, 356 μmol) gave N-[(1R)-1-[3-[3,5-bis(hydroxymethyl)phenyl]phenyl]ethyl]-2-methyl-5-(4-methylpiperazin-1-yl)benzamide (75 mg, 42%) as a colourless crystalline solid. ¹H NMR (500 MHz, DMSO-d₆) δ 8.76 (d, J=8.0, 1H), 7.72 (br s, 1H), 7.55 (d, J=7.5, 1H), 7.50 (br s, 2H), 7.47 (t, J=7.5, 1H), 7.41 (br d, J=7.5, 1H), 7.33 (br s, 1H), 7.10 (d, J=8.5, 1H), 6.94 (dd, J=8.5, 3.0, 1H), 6.89 (d, J=3.0, 1H), 5.27 (t, J=5.5, 2H), 5.21 (quin, J=7.0, 1H), 4.60 (d, J=5.5, 4H), 3.13 (m, 4H), 2.47 (m, 4H), 2.25 (s, 3H), 2.21 (s, 3H), 1.51 (d, J=7.0, 3H). LC-MS (Method B): R_{T}=3.10, m/z=472.9 [M−H]⁻.

### Example 82: 3-[3-[(1R)-1-[[2-Methyl-5-(4-methylpiperazin-1-yl)benzoyl]amino]ethyl]phenyl] propanoic Acid

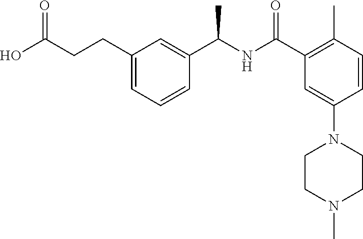 [SMILES: Cc1ccc(N2CCN(C)CC2)cc1C(=O)N[C@H](C)c1cccc(CCC(=O)O)c1]

### Step A: Benzyl (E)-3-[3-[(1R)-1-[[2-methyl-5-(4-methylpiperazin-1-yl)benzoyl]amino]ethyl]phenyl] prop-2-enoate

Under inert atmosphere, N-[(1R)-1-(3-bromophenyl)ethyl]-2-methyl-5-(4-methylpiperazin-1-yl)benzamide (315 mg, 757 μmol) (Example 22), benzyl acrylate (245 mg, 1.51 mmol, 232 μL), tetrabutylammonium chloride (421 mg, 1.51 mmol), sodium hydrogen carbonate (127 mg, 1.51 mmol) and palladium(II) acetate (8.5 mg, 37.8 μmol) were suspended in DMF (4.8 mL) and the reaction mixture heated to 100° C. for 2 hours. The reaction mixture was allowed to cool to RT and 2M potassium carbonate (10 mL) and ethyl acetate (50 mL) were added. The organic phase was washed with brine (100 mL), dried (Na₂SO₄) and the solvent was removed in vacuo. Purification by FCC (eluting with 0-20% 1M NH₃ in MeOH in DCM) gave benzyl (E)-3-[3-[(1R)-1-[[2-methyl-5-(4-methylpiperazin-1-yl)benzoyl]amino]ethyl]phenyl]prop-2-enoate (279 mg, 74%) as a beige foam. ¹H NMR (500 MHz, CDCl₃) δ 7.76 (d, J=15.9, 1H), 7.52 (s, 1H), 7.47-7.31 (m, 8H), 7.11-7.04 (m, 1H), 6.92 (d, J=2.6, 1H), 6.90-6.81 (m, 1H), 6.50 (d, J=15.9, 1H), 6.00-5.93 (m, 1H), 5.37-5.28 (m, 1H), 5.24 (s, 2H), 3.21-3.09 (m, 4H), 2.60-2.50 (m, 4H), 2.34 (s, 3H), 2.31 (s, 3H), 1.58 (d, 3H). LC-MS (Method A): R_{T}=4.76, m/z=498.8 [M+H]⁺.

### Step B: 3-[3-[(1R)-1-[[2-Methyl-5-(4-methylpiperazin-1-yl)benzoyl]amino]ethyl]phenyl]propanoic Acid

Using General Procedure 5 with palladium hydroxide, 20% on activated carbon powder (115 mg) and benzyl (E)-3-[3-[(1R)-1-[[2-methyl-5-(4-methylpiperazin-1-yl)benzoyl]amino]ethyl]phenyl]prop-2-enoate (279 mg, 561 μmol) gave 3-[3-[(1R)-1-[[2-methyl-5-(4-methylpiperazin-1-yl)benzoyl]amino]ethyl]phenyl]propanoic acid (229 mg, 89%) as a yellow foam. ¹H NMR (500 MHz, MeOH-d₄) δ 7.20 (s, 1H), 7.17-7.08 (m, 3H), 7.08-6.97 (m, 3H), 6.91-6.85 (m, 1H), 6.85-6.81 (m, 1H), 5.14-5.03 (m, 1H), 3.30-3.14 (m, 4H), 2.95-2.87 (m, 4H), 2.86-2.80 (m, 2H), 2.54 (s, 3H), 2.50-2.43 (m, 2H), 2.16 (s, 3H), 1.41 (d, 3H). LC-MS (Method A): R_{T}=2.80, m/z=410.7 [M+H]⁺.

### Example 83: 3-[3-[(1R)-1-[[2-Methyl-5-(4-methylpiperazin-1-yl)benzoyl]amino]ethyl]phenyl]benzoic Acid

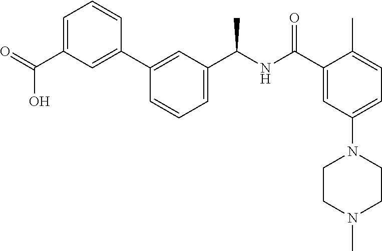 [SMILES: Cc1ccc(N2CCN(C)CC2)cc1C(=O)N[C@H](C)c1cccc(-c2cccc(C(=O)O)c2)c1]

Using General Procedure 5 with benzyl 3-[3-[(1R)-1-[[2-methyl-5-(4-methylpiperazin-1-yl)benzoyl]amino]ethyl]phenyl] benzoate (2.20 g, 4.02 mmol)—prepared in a similar manner to N-[(1R)-1-[3-(4-methoxyphenyl)phenyl]ethyl]-2-methyl-5-(4-methylpiperazin-1-yl)benzamide (Example 40)—and palladium, 10% on activated carbon powder (213 mg, 2.01 mmol) gave 3-[3-[(1R)-1-[[2-methyl-5-(4-methylpiperazin-1-yl)benzoyl]amino]ethyl]phenyl] benzoic acid (1.48 g, 81%) as a beige solid. ¹H NMR (500 MHz, CDCl₃) δ 8.32 (t, J=1.7, 1H), 8.02 (td, J=1.2, 7.6, 1H), 7.79-7.64 (m, 2H), 7.56-7.52 (m, J=7.6, 1H), 7.49 (t, J=7.6, 1H), 7.47-7.40 (m, 1H), 7.35 (d, J=7.6, 1H), 7.29-7.23 (m, 1H), 7.15 (d, J=8.2, 1H), 7.11-6.96 (m, 1H), 6.19 (br d, J=7.6, 1H), 5.38 (quin, J=6.7, 1H), 3.37-3.28 (m, 2H), 3.28-3.20 (m, 2H), 2.95 (br s, 4H), 2.68 (s, 3H), 2.41 (s, 3H), 1.62 (d, J=6.7, 3H). LC-MS (Method B): R_{T}=2.00, m/z=456.7 [M−H]⁻.

### Example 84: N-[(1R)-1-[3-[3-(Dimethylamino)-3-oxo-propyl]phenyl]ethyl]-2-methyl-5-(4-methyl piperazin-1-yl)benzamide

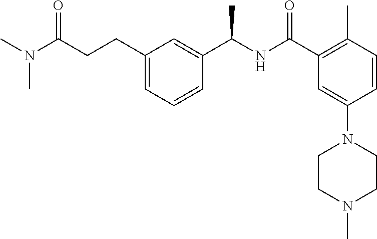 [SMILES: Cc1ccc(N2CCN(C)CC2)cc1C(=O)N[C@H](C)c1cccc(CCC(=O)N(C)C)c1]

Using General Procedure 1 with 3-[3-[(1R)-1-[[2-methyl-5-(4-methylpiperazin-1-yl)benzoyl]amino]ethyl]phenyl]propanoic acid (77 mg, 188 μmol) (Example 82) and N-methylmethanamine (42 mg, 0.94 mmol) with purification by FCC (eluting with 0-50% MeOH in DCM) gave N-[(1R)-1-[3-[3-(dimethylamino)-3-oxo-propyl]phenyl]ethyl]-2-methyl-5-(4-methylpiperazin-1-yl)benzamide (80 mg, 93%) as a pale yellow foam solid. ¹H NMR (500 MHz, CDCl₃) δ 7.31-7.19 (m, 3H), 7.17-7.12 (m, 1H), 7.10-7.05 (m, 1H), 6.96-6.90 (m, 1H), 6.90-6.83 (m, 1H), 6.02-5.89 (m, 1H), 5.33-5.23 (m, 1H), 3.23-3.11 (m, 4H), 3.01-2.90 (m, 8H), 2.65-2.58 (m, 2H), 2.58-2.52 (m, 4H), 2.34 (s, 3H), 2.31 (s, 3H), 1.58 (d, 3H). LC-MS (Method A): R_{T}=2.95, m/z=437.8 [M+H]⁺.

### Further Examples

The following examples were prepared in a similar manner to N-[(1R)-1-[3-[3-(dimethylamino)-3-oxo-propyl]phenyl]ethyl]-2-methyl-5-(4-methyl piperazin-1-yl)benzamide (Example 84), using the intermediate example carboxylic acid stated and the required commercially available amine.

| Example | | |
|---|---|---|
| (Intermediate) | Structure | Name and Analytical Data |
| 85 (Example 82) | 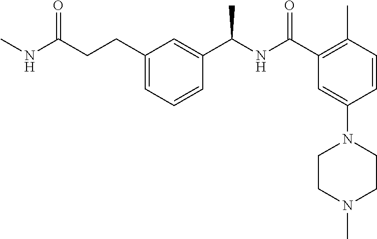 [SMILES: CNC(=O)CCc1cccc([C@@H](C)NC(=O)c2cc(N3CCN(C)CC3)ccc2C)c1] | 2-Methyl-N-[(1R)-1-[3-[3-(methylamino)- 3-oxo-propyl]phenyl]ethyl]-5-(4-methyl piperazin-1-yl)benzamide ¹H NMR (500 MHz, CDCl₃) δ 7.31-7.27 (m, 1H), 7.23-7.17 (m, 2H), 7.14-7.10 (m, 1H), 7.10-7.05 (m, 1H), 6.94-6.90 (m, 1H), 6.90- 6.83 (m, 1H), 6.01-5.91 (m, 1H), 5.50-5.35 (m, 1H), 5.32-5.21 (m, 1H), 3.20-3.10 (m, 4H), 3.01-2.93 (m, 2H), 2.77-2.71 (m, 3H), 2.60-2.53 (m, 4H), 2.49-2.43 (m, 2H), 2.35 (s, 3H), 2.26 (s, 3H), 2.07 (d, 1H). LC-MS (Method A): R_{T} = 2.85, m/z = 423.8 [M + H]⁺. |
| 86 (Example 83) | 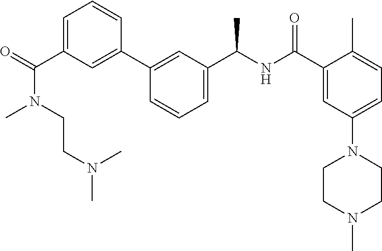 [SMILES: Cc1ccc(N2CCN(C)CC2)cc1C(=O)N[C@H](C)c1cccc(-c2cccc(C(=O)N(C)CCN(C)C)c2)c1] | N-[(1R)-1-[3-[3-[2-(Dimethylamino)ethyl- methyl- carbamoyl]phenyl]phenyl]ethyl]-2- methyl-5-(4-methylpiperazin-1- yl)benzamide ¹H NMR (500 MHz, DMSO-d₆) δ 8.71 (d, J = 8.0, 1H), 7.73 (m, 2H), 7.63 (m, 1H), 7.57 (br d, J = 7.5, 1H), 7.53 (t, J = 7.5, 1H), 7.45 (t, J = 7.5, 1H), 7.41 (br d, J = 7.5, 1H), 7.36 (d, J = 7.5, 1H), 7.06 (d, J = 8.5, 1H), 6.91 (dd, J = 8.5, 2.5, 1H), 6.85 (d, J = 2.5, 1H), 5.19 (quin, J = 7.0, 1H), 3.56 (m, 1H), 3.30 (m, 1H), 3.10 (m, 4H), 2.99-2.94 (m, 3H), 2.44 (m, 4H), 2.37 (m, 1H), 2.22 (br s, 5H), 2.17 (s, 3H), 1.95 (br s, 3H), 1.48 (d, J = 7.0, 3H). LC-MS (Method B): R_{T} = 3.23, m/z = 540.9 [M − H]⁻. |
| 87 (Example 83) | 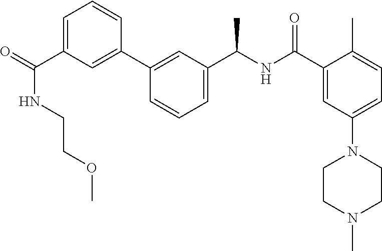 [SMILES: COCCNC(=O)c1cccc(-c2cccc([C@@H](C)NC(=O)c3cc(N4CCN(C)CC4)ccc3C)c2)c1] | N-[(1R)-1-[3-[3-(2- Methoxyethylcarbamoyl)phenyl]phenyl] ethyl]-2-methyl-5-(4-methylpiperazin-1- yl)benzamide ¹H NMR (500 MHz, DMSO-d₆) δ 8.73 (d, J = 8.0, 1H), 8.66 (t, J = 5.0, 1H), 8.14 (m, 1H), 7.85 (d, J = 8.0, 1H), 7.81 (d, J = 8.0, 1H), 7.76 (br s, 1H) 7.61 (d, J = 7.5, 1H), 7.57 (t, J = 7.5, 1H), 7.48 (t, J = 7.5, 1H), 7.42 (br d, J = 7.5, 1H), 7.08 (d, J = 8.0, 1H), 6.93 (dd, J = 8.5, 2.5, 1H), 6.87 (d, J = 3.0, 1H), 5.20 (quin, J = 7.5, 1H), 3.50-3.44 (m, 4H), 3.28-3.26 (m, 4H), 3.16-3.14 (m, 4H), 2.37 (br s, 3H), 2.18 (s, 3H), 1.49 (d, J = 7.5, 3H). LC-MS (Method B): R_{T} = 3.20, m/z = 513.8 [M − H]⁻. |
| 88 (Example 83) | 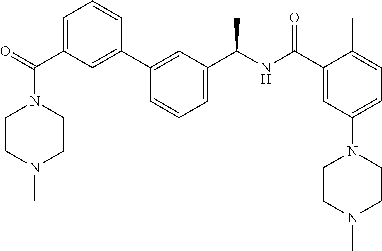 [SMILES: Cc1ccc(N2CCN(C)CC2)cc1C(=O)N[C@H](C)c1cccc(-c2cccc(C(=O)N3CCN(C)CC3)c2)c1] | 2-Methyl-N-[(1R)-1-[3-[3-(4- methylpiperazine-1- carbonyl)phenyl]phenyl]ethyl]-5-(4- methylpiperazin-1-yl)benzamide ¹H NMR (500 MHz, DMSO-d₆) δ 8.71 (d, J = 8.0, 1H), 7.76-7.74 (m, 2H), 7.64 (t, J = 1.5, 1H), 7.58-7.53 (m, 2H), 7.45 (t, J = 7.5, 1H), 7.41 (br d, J = 7.5, 1H), 7.38 (dt, J = 7.5, 1.5, 1H), 7.06 (d, J = 8.5, 1H), 6.91 (dd, J = 8.5, 2.5, 1H), 6.86 (d, J = 2.5, 1H), 5.19 (quin, J = 7.5, 1H), 3.64 (br s, 2H), 3.35 (br s, 4H), 3.11-3.09 (m, 4H), 2.47-2.43 (m, 4H), 2.37 (br s, 2H), 2.24 (s, 3H), 2.20 (s, 3H), 2.17 (s, 3H), 1.48 (d, J = 7.0, 3H). LC- MS (Method B): R_{T} = 3.20, m/z = 538.9 [M − H]⁻. |
| 89 (Example 83) | 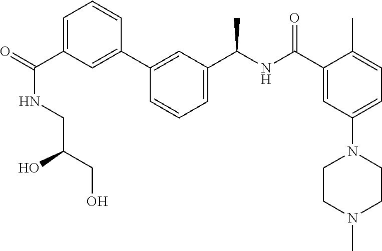 [SMILES: Cc1ccc(N2CCN(C)CC2)cc1C(=O)N[C@H](C)c1cccc(-c2cccc(C(=O)NC[C@H](O)CO)c2)c1] | N-[(1R)-1-[3-[3-[(2S)-2,3- Dihydroxypropyl]carbamoyl]phenyl]phenyl] ethyl]-2-methyl-5-(4- methylpiperazin-1-yl)benzamide ¹H NMR (500 MHz, DMSO-d₆) δ 8.73 (d, J = 8.0, 1H), 8.55 (t, J = 6.0, 1H), 8.15 (s, 1H), 7.86 (d, J = 7.5, 1H), 7.81 (d, J = 8.0, 1H), 7.76 (br s 1H), 7.61 (d, J = 7.5, 1H), 7.56 (t, J = 8.0, 1H), 7.48 (m, 1H), 7.43 (d, J = 7.5, 1H), 7.07 (d, J = 8.5, 1H), 6.91 (dd, J = 8.5, 2.5, 1H), 6.86 (d, J = 2.5, 1H), 5.20 (quin, J = 7.0, 1H), 4.84 (d, J = 4.5, 1H), 4.59-4.56 (m, 1H), 3.67 (dq, J = 11.0, 5.5, 1H), 3.43 (dt, J = 13.0, 5.5, 1H), 3.37 (t, J = 5.0, 2H), 3.24- 3.21 (m, 1H), 3.11-3.09 (m, 4H), 2.26 (s, 3H), 2.17 (s, 3H), 1.49 (d, J = 7.0, 3H). LC- MS (Method B): R_{T} = 2.88, m/z = 529.9 [M − H]⁻. |
| 90 (Example 83) | 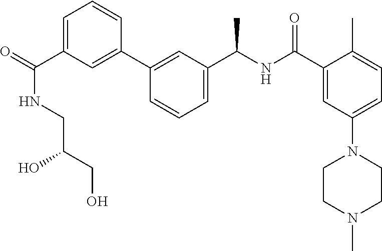 [SMILES: Cc1ccc(N2CCN(C)CC2)cc1C(=O)N[C@H](C)c1cccc(-c2cccc(C(=O)NC[C@@H](O)CO)c2)c1] | N-[(1R)-1-[3-[3-[(2R)-2,3- Dihydroxypropyl]carbamoyl]phenyl]phenyl] ethyl]-2-methyl-5-(4- methylpiperazin-1-yl)benzamide ¹H NMR (500 MHz, CDCl₃) δ 7.98 (s, 1H), 7.72 (br d, J = 7.6, 1H), 7.69-7.64 (m, 1H), 7.64-7.57 (m, 1H), 7.48-7.37 (m, 4H), 7.37- 7.33 (m, 1H), 7.04 (d, J = 8.2, 1H), 6.91-6.80 (m, 2H), 6.50 (br d, J = 7.9, 1H), 5.30 (quin, J = 7.1, 1H), 3.87-3.72 (m, 1H), 3.58-3.43 (m, 5H), 3.08 (brs, 5H), 2.51 (brs, 4H), 2.35- 2.23 (m, 6H), 1.58 (d, J = 7.0, 3H). LC- MS (Method B): R_{T} = 2.96, m/z = 529.9 [M − H]⁻. |
| 91 (Example 83) | 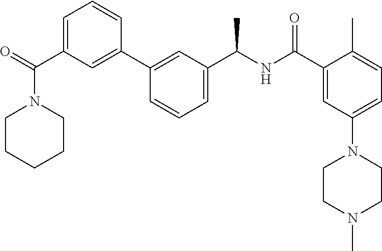 [SMILES: Cc1ccc(N2CCN(C)CC2)cc1C(=O)N[C@H](C)c1cccc(-c2cccc(C(=O)N3CCCCC3)c2)c1] | 2-Methyl-5-(4-methylpiperazin-1-yl)-N- [(1R)-1-[3-[3-(piperidine-1- carbonyl)phenyl]phenyl]ethyl]benzamide ¹H NMR (500 MHz, CDCl₃) δ 7.66-7.55 (m, 3H), 7.52-7.41 (m, 3H), 7.40-7.33 (m, 2H), 7.08 (d, J = 8.5, 1H), 6.93 (d, J = 2.4, 1H), 6.87 (dd, J = 2.3, 8.4, 1H), 6.08 (br d, J = 7.6, 1H), 5.38 (quin, J = 7.1, 1H), 3.73 (br s, 2H), 3.38 (br s, 2H), 3.20-3.10 (m, 4H), 2.60- 2.50 (m, 4H), 2.33 (d, J = 7.9, 6H), 1.68 (br s, 4H), 1.66-1.60 (m, 3H), 1.56-1.48 (m, 2H). LC-MS (Method B): R_{T} = 3.53, m/z = 523.9 [M − H]⁻. |
| 92 (Example 83) | 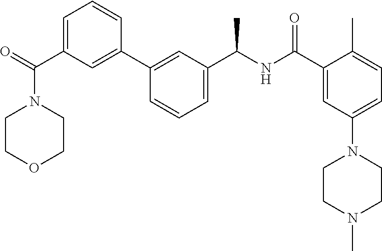 [SMILES: Cc1ccc(N2CCN(C)CC2)cc1C(=O)N[C@H](C)c1cccc(-c2cccc(C(=O)N3CCOCC3)c2)c1] | 2-Methyl-5-(4-methylpiperazin-1-yl)-N- [(1R)-1-[3-[3-(morpholine-4- carbonyl)phenyl]phenyl]ethyl]benzamide ¹H NMR (500 MHz, CDCl₃) δ 7.68-7.56 (m, 3H), 7.52-7.42 (m, 3H), 7.41-7.35 (m, 2H), 7.08 (d, J = 8.2, 1H), 6.93 (d, J = 2.4, 1H), 6.91-6.84 (m, 1H), 6.06 (br d, J = 7.9, 1H), 5.43-5.32 (m, 1H), 3.92-3.40 (m, 8H), 3.23- 3.09 (m, 4H), 2.60-2.52 (m, 4H), 2.34 (s, 3H), 2.32 (s, 3H), 1.63 (d, J = 6.7, 3H). LC- MS (Method B): R_{T} = 3.22, m/z = 525.9 [M − H]⁻. |
| 93 (Example 83) | 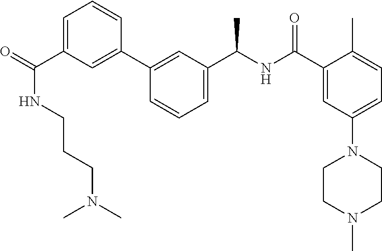 [SMILES: Cc1ccc(N2CCN(C)CC2)cc1C(=O)N[C@H](C)c1cccc(-c2cccc(C(=O)NCCCN(C)C)c2)c1] | N-[(1R)-1-[3-[3-[3- (Dimethylamino)propylcarbamoyl]phenyl] phenyl]ethyl]-2-methyl-5-(4- methylpiperazin-1-yl)benzamide ¹H NMR (500 MHz, CDCl₃) δ 8.55 (br s, 1H), 8.03 (s, 1H), 7.74 (d, J = 7.6, 1H), 7.72- 7.67 (m, 1H), 7.62 (s, 1H), 7.55-7.42 (m, 3H), 7.42-7.36 (m, 1H), 7.08 (d, J = 8.5, 1H), 6.93 (d, J = 2.4, 1H), 6.90-6.84 (m, 1H), 6.01 (br d, J = 7.9, 1H), 5.43-5.34 (m, 1H), 3.65- 3.53 (m, 2H), 3.22-3.09 (m, 4H), 2.57-2.50 (m, 6H), 2.35-2.33 (s, 3H), 2.33-2.31 (s, 3H), 2.30 (s, 6H), 1.79 (td, J = 6.0, 11.9, 2H), 1.64 (d, J = 7.0, 3H). LC-MS (Method B): R_{T} = 3.68 min m/z = 541.0 [M − H]⁻. |
| 94 (Example 83) | 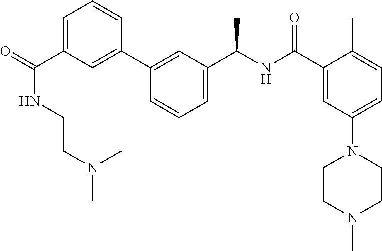 [SMILES: Cc1ccc(N2CCN(C)CC2)cc1C(=O)N[C@H](C)c1cccc(-c2cccc(C(=O)NCCN(C)C)c2)c1] | N-[(1R)-1-[3-[3-[2- (Dimethylamino)ethylcarbamoyl]phenyl] phenyl]ethyl]-2-methyl-5-(4- methylpiperazin-1-yl)benzamide ¹H NMR (500 MHz, CDCl₃) δ 8.04 (t, J = 1.7, 1H), 7.75 (d, J = 7.6, 1H), 7.73-7.68 (m, 1H), 7.63 (s, 1H), 7.55-7.43 (m, 3H), 7.43-7.36 (m, 1H), 7.08 (d, J = 8.2, 1H), 6.96-6.90 (m, 2H), 6.90-6.85 (m, 1H), 6.01 (br d, J = 7.9, 1H), 5.44-5.35 (m, 1H), 3.60-3.50 (m, 2H), 3.19-3.12 (m, 4H), 2.57-2.52 (m, 6H), 2.30- 2.36 (m, 6H), 2.31-2.24 (m, 3H), 1.66-1.60 (m, 6H). LC-MS (Method B): R_{T} = 3.29, m/z = 526.9 [M − H]⁻. |
| 95 (Example 83) | 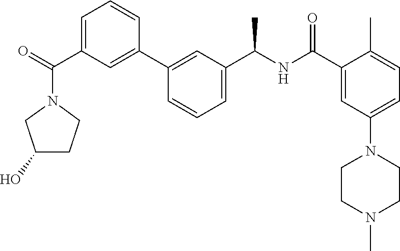 [SMILES: Cc1ccc(N2CCN(C)CC2)cc1C(=O)N[C@H](C)c1cccc(-c2cccc(C(=O)N3CC[C@H](O)C3)c2)c1] | N-[(1R)-1-[3-[3-[(3S)-3- Hydroxypyrrolidine-1- carbonyl]phenyl]phenyl]ethyl]-2- methyl-5-(4-methylpiperazin-1- yl)benzamide ¹H NMR (500 MHz, CDCl₃) δ 7.73 (br d, J = 6.1, 1H), 7.66-7.57 (m, 2H), 7.52-7.40 (m, 4H), 7.40-7.35 (m, 1H), 7.07 (br d, J = 8.5, 1H), 6.94-6.83 (m, 2H), 6.30-6.13 (m, 1H), 5.43-5.31 (m, 1H), 4.60-4.35 (m, 1H), 3.91-3.32 (m, 5H), 3.22-3.03 (m, 4H), 2.65-2.49 (m, 4H), 2.40-2.20 (m, 8H), 1.62 (t, J = 6.0, 3H). LC-MS (Method B): R_{T} = 3.30, m/z = 526.0 [M − H]⁻. |

### Example 96: N-[(1R)-1-[3-[3-(2-Hydroxyethylamino)-3-oxo-propyl]phenyl]ethyl]-2-methyl-5-(4-methylpiperazin-1-yl)benzamide

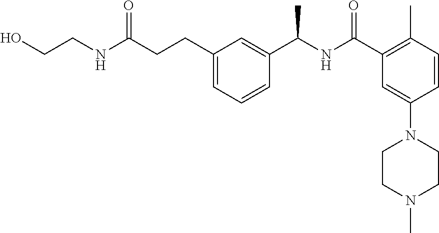 [SMILES: Cc1ccc(N2CCN(C)CC2)cc1C(=O)N[C@H](C)c1cccc(CCC(=O)NCCO)c1]

Using General Procedure 4 with N-[(1R)-1-[3-[3-[2-[tert-butyl(dimethyl)silyl]oxyethylamino]-3-oxo-propyl]phenyl]ethyl]-2-methyl-5-(4-methylpiperazin-1-yl)benzamide (112 mg, 197 μmol)—prepared in a similar manner to N-[(1R)-1-[3-[3-(dimethylamino)-3-oxo-propyl]phenyl]ethyl]-2-methyl-5-(4-methylpiperazin-1-yl)benzamide (Example 84)—gave N-[(1R)-1-[3-[3-(2-hydroxyethylamino)-3-oxo-propyl]phenyl]ethyl]-2-methyl-5-(4-methylpiperazin-1-yl)benzamide (72 mg, 76%) as a pale yellow foam solid. ¹H NMR (500 MHz, CDCl₃) δ 7.31-7.27 (m, 1H), 7.24-7.22 (m, 1H), 7.21-7.17 (m, 1H), 7.14-7.06 (m, 2H), 6.94-6.91 (m, 1H), 6.91-6.86 (m, 1H), 6.17 (d, 1H), 5.91-5.78 (m, 1H), 5.25-5.15 (m, 1H), 3.53-3.44 (m, 2H), 3.32-3.24 (m, 2H), 3.21-3.12 (m, 4H), 3.02-2.95 (m, 2H), 2.61-2.55 (m, 4H), 2.55-2.43 (m, 2H), 2.33 (s, 3H), 2.31 (s, 3H), 1.56 (d, 3H). LC-MS (Method A): R_{T}=2.52, m/z=453.8 [M+H]⁺.

### Example 97: N-[(1R)-1-[3-[3-(2-Hydroxyethylcarbamoyl)phenyl]phenyl]ethyl]-2-methyl-5-(4-methylpiperazin-1-yl)benzamide

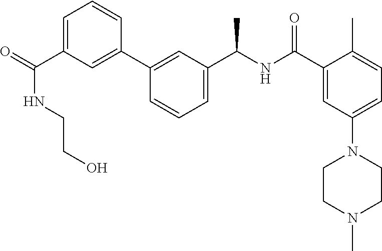 [SMILES: Cc1ccc(N2CCN(C)CC2)cc1C(=O)N[C@H](C)c1cccc(-c2cccc(C(=O)NCCO)c2)c1]

Using General Procedure 4 with N-[(1R)-1-[3-[3-[2-[tert-butyl(dimethyl)silyl]oxyethylcarbamoyl]phenyl]phenyl]ethyl]-2-methyl-5-(4-methylpiperazin-1-yl)benzamide (56 mg, 91 μmol)—prepared in a similar manner to N-[(1R)-1-[3-[3-(2-methoxyethylcarbamoyl)phenyl]phenyl]ethyl]-2-methyl-5-(4-methylpiperazin-1-yl)benzamide (Example 87)—gave N-[(1R)-1-[3-[3-(2-hydroxyethylcarbamoyl)phenyl]phenyl]ethyl]-2-methyl-5-(4-methylpiperazin-1-yl)benzamide (20 mg, 42%) as a white solid. ¹H NMR (500 MHz, DMSO-d₆) δ 8.73 (d, J=8.0, 1H), 8.58 (t, J=5.5, 1H), 8.14 (t, J=1.5, 1H), 7.85 (d, J=7.5, 1H), 7.80 (m, 1H), 7.76 (br s, 1H), 7.61 (d, 17.5, 1H), 7.56 (t, J=7.5, H), 7.47 (t, J=7.5, 1H), 7.42 (d, J=7.5, 1H), 7.06 (d, J=8.5, 1H), 6.91 (dd, J=8.0, 2.5, 1H), 6.86 (d, J=2.5, 1H), 5.20 (quin, 7.0, 1H), 4.75 (br s, 1H), 3.54-3.51 (m, 2H), 3.37 (q, J=6.0, 2H), 3.09-3.06 (m, 4H), 2.44-2.41 (m, 4H), 2.22 (s, 3H), 2.18 (s, 3H), 1.49 (d, J=7.0, 3H). LC-MS (Method B): R_{T}=3.18, m/z=499.9 [M−H]⁻.

### Further Examples

The following examples were prepared in a similar manner to N-[(1R)-1-[3-[3-(2-hydroxyethylcarbamoyl)phenyl]phenyl]ethyl]-2-methyl-5-(4-methylpiperazin-1-yl)benzamide (Example 97).

| Example | Structure | Name and Analytical Data |
|---|---|---|
| 98 | 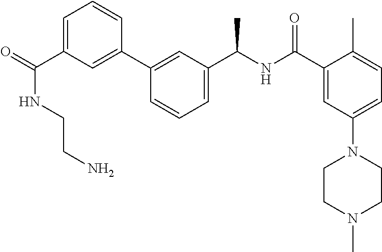 [SMILES: Cc1ccc(N2CCN(C)CC2)cc1C(=O)N[C@H](C)c1cccc(-c2cccc(C(=O)NCCN)c2)c1] | N-[(1R)-1-[3-[3-(2- Aminoethylcarbamoyl)phenyl]phenyl]eth- yl]-2-methyl-5-(4-methylpiperazin-1- yl)benzamide ¹H NMR (500 MHz, DMSO-d₆) δ 8.73 (d, J = 8.0, 1H), 8.53 (d, J = 5.5, 1H), 8.13 (m, 1H), 7.85 (d, J = 7.5, 1H), 7.80-7.79 (m, 1H), 7.76-7.74 (m, 1H), 7.60 (d, J = 7.5, 1H), 7.56 (t, J = 8.0, 1H), 7.47 (t, J = 7.5, 1H), 7.43 (d, J = 7.5, 1H), 7.06 (d, J = 8.5, 1H), 6.91 (dd, J = 8.0, 3.5 1H), 6.86 (d, J = 2.5, 1H), 5.20 (quin, J = 7.0, 1H), 3.30-3.28 (m, 2H), 3.09- 3.07 (m, 4H), 2.71 (t, J = 6.5, 2H), 2.43-2.41 (m, 4H), 2.22 (s, 3H), 2.18 (s, 3H), 1.49 (d, J = 7.0, 3H). LC-MS (Method B): R_{T} = 3.18, m/z = 498.9 [M − H]⁻. |

### Example 99: 2-Methyl-N-[(1R)-1-[3-[3-[methyl-[2-(methylamino)ethyl]carbamoyl]phenyl]phenyl] ethyl]-5-(4-methylpiperazin-1-yl)benzamide Hydrochloride Salt

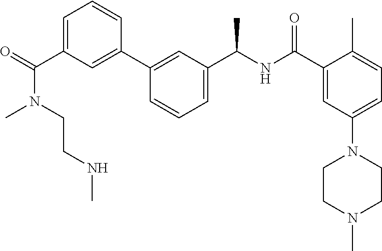 [SMILES: CNCCN(C)C(=O)c1cccc(-c2cccc([C@@H](C)NC(=O)c3cc(N4CCN(C)CC4)ccc3C)c2)c1]

Using General Procedure 5 with benzyl N-methyl-N-[2-[methyl-[3-[3-[(1R)-1-[[2-methyl-5-(4-methylpiperazin-1-yl)benzoyl]amino]ethyl]phenyl]benzoyl]amino]ethyl]carbamate (200 mg, 302 μmol)—prepared in a similar manner to N-[(1R)-1-[3-[3-[3-(dimethylamino)propylcarbamoyl]phenyl]phenyl]ethyl]-2-methyl-5-(4-methylpiperazin-1-yl)benzamide (Example 93)—and palladium, 10% on activated carbon powder (16 mg, 151 μmol) afforded a clear gum. This was dissolved in diethyl ether (10 ml), to this was added 2N HCl in diethyl ether to afford a cloudy solution. This was diluted with DCM to afford a solid which was filtered and dried under nitrogen to afford 2-methyl-N-[(1R)-1-[3-[3-[methyl-[2-(methylamino)ethyl]carbamoyl]phenyl]phenyl]ethyl]-5-(4-methylpiperazin-1-yl)benzamide (44 mg, 42%). ¹H NMR (500 MHz, D₂O) δ 7.79-7.73 (m, 1H), 7.69 (s, 1H), 7.65 (s, 1H), 7.60-7.47 (m, 3H), 7.46-7.41 (m, 2H), 7.19 (d, J=8.2, 1H), 7.04 (dd, J=2.6, 8.4, 1H), 6.93 (d, J=2.4, 1H), 5.16-5.09 (m, 1H), 3.83 (t, J=6.0, 2H), 3.77-3.61 (m, 3H), 3.59-3.52 (m, 2H), 3.33 (t, J=5.8, 2H), 3.23-3.11 (m, 3H), 3.10-2.97 (m, 5H), 2.88 (s, 3H), 2.73 (s, 3H), 2.13 (s, 3H), 1.52 (d, J=7.3, 3H). LC-MS (Method B): RT=3.33, m/z=526.9 [M−H]⁻.

### Example 100: N-(2-Methoxyethyl)-4-[3-[(1R)-1-[[2-methyl-5-(4-methylpiperazin-1-yl)benzoyl]amino]ethyl]phenyl]thiophene-2-carboxamide

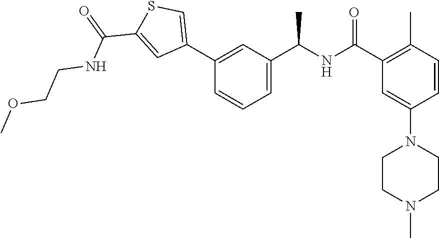 [SMILES: COCCNC(=O)c1cc(-c2cccc([C@@H](C)NC(=O)c3cc(N4CCN(C)CC4)ccc3C)c2)cs1]

### Step A: 4-[3-[(1R)-1-[[2-Methyl-5-(4-methylpiperazin-1-yl)benzoyl]amino]ethyl]phenyl]thiophene-2-carboxylic Acid Hydrochloride Salt

Using General Procedure 2 with N-[(1R)-1-(3-bromophenyl)ethyl]-2-methyl-5-(4-methylpiperazin-1-yl)benzamide (600 mg, 1.44 mmol) (Example 22) and 4-boronothiophene-2-carboxylic acid (297 mg, 1.73 mmol) at 85° C. overnight gave 4-[3-[(1R)-1-[[2-methyl-5-(4-methylpiperazin-1-yl)benzoyl]amino]ethyl]phenyl]thiophene-2-carboxylic acid hydrochloride salt (250 mg, 35%) as a beige solid which was used without further purification.

### Step B: N-(2-Methoxyethyl)-4-[3-[(1R)-1-[[2-methyl-5-(4-methylpiperazin-1-yl)benzoyl]amino]ethyl]phenyl]thiophene-2-carboxamide

Using General Procedure 1 with 2-methoxyethanamine (15 mg, 200 μmol) and 4-[3-[(1R)-1-[[2-methyl-5-(4-methylpiperazin-1-yl)benzoyl]amino]ethyl]phenyl]thiophene-2-carboxylic acid hydrochloride salt (50 mg, 100 μmol) gave N-(2-methoxyethyl)-4-[3-[(1R)-1-[[2-methyl-5-(4-methylpiperazin-1-yl)benzoyl]amino]ethyl]phenyl]thiophene-2-carboxamide (11 mg, 20%) as a white crystalline solid. ¹H NMR (500 MHz, DMSO-d₆) δ 8.68 (d, J=8.0, 1H), 8.64 (t, J=5.5, 1H), 8.25 (d, J=1.5, 1H), 8.03 (d, J=1.5, 1H), 7.73 (m, 1H), 7.56 (d, J=7.5, 1H), 7.43 (t, J=7.5, 1H), 7.36 (br d, J=8.0, 1H), 7.06 (d, J=8.5, 1H), 6.91 (dd, J=8.0, 2.5, 1H), 6.86 (d, J=2.5, 1H), 5.16 (quin, J=7.0, 1H), 3.49-3.47 (m, 2H), 3.45-3.41 (m, 2H), 3.29 (s, 3H), 3.12 (br s, 4H), 2.46 (br s, 4H), 2.24 (br s, 3H), 2.16 (s, 3H), 1.48 (d, J=7.0, 3H). LC-MS (Method B): R_{T}=3.42, m/z=520.0 [M−H]⁻.

### Further Examples

The following examples were prepared in a similar manner to N-(2-methoxyethyl)-4-[3-[(1R)-1-[[2-methyl-5-(4-methylpiperazin-1-yl)benzoyl]amino]ethyl]phenyl]thiophene-2-carboxamide (Example 100) using the required commercially available amine.

| Example | Structure | Name and Analytical Data |
|---|---|---|
| 101 | 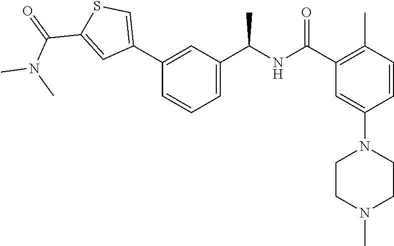 [SMILES: Cc1ccc(N2CCN(C)CC2)cc1C(=O)N[C@H](C)c1cccc(-c2csc(C(=O)N(C)C)c2)c1] | N,N-Dimethyl-4-[3-[(1R)-1-[[2-methyl-5- (4-methylpiperazin-1- yl)benzoyl]amino]ethyl]phenyl]thiophene- 2-carboxamide ¹H NMR (500 MHz, DMSO-d₆) δ 8.66 (d, J = 8.0, 1H), 8.02 (d, J 1.5, 1H), 7.87 (d, J = 1.5, 1H), 7.78 (m, 1H), 7.62 (d, J = 7.5, 1H), 7.40 (t, J = 7.5, 1H), 7.35 (br d, J = 7.5, 1H), 7.09 (d, J = 8.5, 1H), 6.94 (dd, J = 8.0, 2.5, 1H), 6.88 (d, J = 2.5, 1H), 5.17 (quin, J = 7.0, 1H), 3.19 (br s, 8H), 2.73 (m, 3H), 2.43 (br s, 3H), 2.18 (s, 3H), 1.47 (d, J = 7.0, 3H). LC-MS (Method B): R_{T} = 3.50, m/z = 490.0 [M − H]⁻. |

### Example 102: N,N,1-Trimethyl-4-[3-[(1R)-1-[[2-methyl-5-(4-methylpiperazin-1-yl)benzoyl]amino]ethyl]phenyl]pyrrole-2-carboxamide

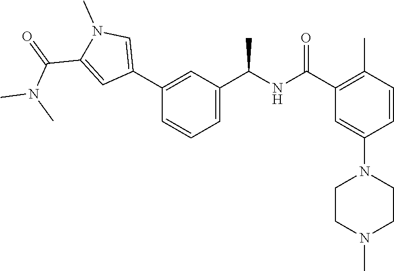 [SMILES: Cc1ccc(N2CCN(C)CC2)cc1C(=O)N[C@H](C)c1cccc(-c2cc(C(=O)N(C)C)n(C)c2)c1]

### Step A: Benzyl 4-bromo-1-methyl-pyrrole-2-carboxylate

Benzyl Bromide (2.91 mL, 24.5 mmol) was added to a solution of 4-bromo-1-methyl-pyrrole-2-carboxylic acid (5.00 g, 24.5 mmol) and potassium carbonate (5.08 g, 36.8 mmol) in DMF (75 mL) and the reaction mixture allowed to stir at RT overnight Water (150 mL) and diethyl ether (150 mL) was added, and the phases separated. The aqueous phase was extracted with petroleum ether (60 mL) and the combined organic phases washed with brine (120 mL), dried over sodium sulfate and the solvent removed in vacuo. Purification by FCC (eluting with 0-50% diethyl ether in petroleum ether) gave benzyl 4-bromo-1-methyl-pyrrole-2-carboxylate (5.32 g, 74%) as an orange oil. ¹H NMR (500 MHz, CDCl₃) δ 7.34-7.24 (m, 5H), 6.89 (d, J=2.0, 1H), 6.70 (d, J=2.0, 1H), 5.19 (s, 2H), 3.83 (s, 3H).

### Step B: Benzyl 1-methyl-4-(4,4,5,5-tetramethyl-1,3,2-dioxaborolan-2-yl)pyrrole-2-carboxylate

1,1′-Bis(diphenylphosphino)ferrocenepalladium (II) dichloride (1.32 g, 1.81 mmol) was added to a degassed solution of benzyl 4-bromo-1-methyl-pyrrole-2-carboxylate (5.32 g, 18.1 mmol), bis(pinacolato)diboron (5.51 g, 21.7 mmol) and potassium pivalate (7.61 g, 54.3 mmol) in 1,4-dioxane (100 mL) and the reaction mixture heated at 85° C. overnight. The reaction mixture was allowed to cool to RT and water (150 mL) and diethyl ether (150 mL) were added and the phases separated. The organic phase was washed with brine (100 mL), dried over sodium sulfate and the solvent removed in vacuo. Purification by FCC (eluting with 0-40% diethyl ether in petroleum ether) gave benzyl 1-methyl-4-(4,4,5,5-tetramethyl-1,3,2-dioxaborolan-2-yl)pyrrole-2-carboxylate (4.65 g, 75%) as a yellow oil. ¹H NMR (500 MHz, CDCl₃) δ 7.34-7.23 (m, 6H), 7.12 (d, J=1.5, 1H), 5.18 (s, 2H), 3.85 (s, 3H), 1.23 (s, 12H).

### Step C: Benzyl 1-methyl-4-[3-[(1R)-1-[[2-methyl-5-(4-methylpiperazin-1-yl)benzoyl]amino]ethyl]phenyl]pyrrole-2-carboxylate

Using General Procedure 2 with N-[(1R)-1-(3-bromophenyl)ethyl]-2-methyl-5-(4-methylpiperazin-1-yl)benzamide (375 mg, 900 μmol) (Example 22) and benzyl 1-methyl-4-(4,4,5,5-tetramethyl-1,3,2-dioxaborolan-2-yl)pyrrole-2-carboxylate (307 mg, 900 μmol) gave benzyl 1-methyl-4-[3-[(1R)-1-[[2-methyl-5-(4-methylpiperazin-1-yl)benzoyl]amino]ethyl]phenyl]pyrrole-2-carboxylate (334 mg, 67%) as a yellow oil. LC-MS (Method B): R_{T}=4.37, m/z=550.0 [M−H]⁻.

### Step D: 1-Methyl-4-[3-[(1R)-1-[[2-methyl-5-(4-methylpiperazin-1-yl)benzoyl]amino]ethyl]phenyl]pyrrole-2-carboxylic Acid

Using General Procedure 5 with palladium hydroxide, 20% on activated carbon (50 mg) and benzyl 1-methyl-4-[3-[(1R)-1-[[2-methyl-5-(4-methylpiperazin-1-yl)benzoyl]amino]ethyl]phenyl]pyrrole-2-carboxylate (334 mg, 607 μmol) gave 1-methyl-4-[3-[(1R)-1-[[2-methyl-5-(4-methylpiperazin-1-yl)benzoyl]amino]ethyl]phenyl]pyrrole-2-carboxylic acid (204 mg, 73%) as an off-white solid. LC-MS (Method B): R_{T}=0.49, m/z=459.8 [M−H]⁻.

### Step E: N,N,1-Trimethyl-4-[3-[(1R)-1-[[2-methyl-5-(4-methylpiperazin-1-yl)benzoyl]amino]ethyl]phenyl]pyrrole-2-carboxamide

Using General Procedure 1 with 1-methyl-4-[3-[(1R)-1-[[2-methyl-5-(4-methylpiperazin-1-yl)benzoyl]amino]ethyl]phenyl]pyrrole-2-carboxylic acid (59 mg, 128 μmol) and dimethylamine (2M in THF, 320 μL gave N,N,1-trimethyl-4-[3-[(1R)-1-[[2-methyl-5-(4-methylpiperazin-1-yl)benzoyl]amino]ethyl]phenyl]pyrrole-2-carboxamide (31 mg, 47%) as a white crystalline solid. ¹H NMR (500 MHz, DMSO-d₆) δ 8.62 (d, J=8.0, 1H), 7.59 (br s, 1H), 7.41 (d, J=8.0, 1H), 7.34 (m, 1H), 7.29 (t, J=7.5, 1H), 7.17 (d, J=8.0, 1H), 7.06 (d, J=8.5, 1H), 6.91 (dd, J=8.5, 2.5, 1H), 6.86 (d, J=2.5, 1H), 6.76 (d, J=2.0, 1H), 5.12 (quin, J=7.0, 1H), 3.71 (s, 3H), 3.09-3.06 (m, 10H), 2.44-2.41 (m, 4H), 2.22 (s, 3H), 2.18 (s, 3H), 1.45 (d, J=7.0, 3H). LC-MS (Method B): R_{T}=3.21, m/z=486.9 [M−H]⁻.

### Example 103: 2-Methyl-5-(4-methylpiperazin-1-yl)-N-[(1R)-1-[3-(4-methylpiperazin-1-yl)phenyl] ethyl]benzamide

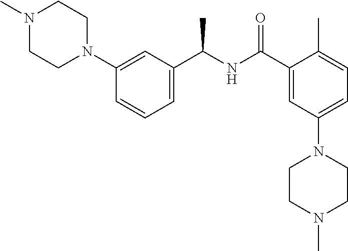 [SMILES: Cc1ccc(N2CCN(C)CC2)cc1C(=O)N[C@H](C)c1cccc(N2CCN(C)CC2)c1]

Using General Procedure 3 with 1-methylpiperazine (92 μL 829 μmol) and N-[(1R)-1-(3-bromophenyl)ethyl]-2-methyl-5-(4-methylpiperazin-1-yl)benzamide (230 mg, 552 μmol) (Example 22) at 100° C. overnight gave 2-methyl-5-(4-methylpiperazin-1-yl)-N-[(1R)-1-[3-(4-methylpiperazin-1-yl)phenyl]ethyl]benzamide (100 mg, 39%) as a pale yellow solid. ¹H NMR (500 MHz, DMSO-d₆) δ 8.58 (d, J=8.5, 1H), 7.16 (t, J=8.0, 1H), 7.06 (d, J=8.5, 1H), 6.99 (br s, 1H), 6.91 (dd, J=8.5, 3.0, 1H), 6.83 (d, J=3.0, 1H), 6.79 (dd, J=8.0, 2.0, 1H), 5.05 (quin, J=7.5, 1H), 3.13 (m, 4H), 3.10 (m, 4H), 2.45 (m, 8H), 2.23 (s, 3H), 2.22 (s, 3H), 2.17 (s, 3H), 1.39 (d, J=7.5, 3H). LC-MS (Method B): R_{T}=2.94, m/z=434.9 [M−H]⁻.

### Further Examples

The following examples were prepared in a similar manner to 2-methyl-5-(4-methylpiperazin-1-yl)-N-[(1R)-1-[3-(4-methylpiperazin-1-yl)phenyl]ethyl]benzamide (Example 103), from the intermediate example stated.

| Example | | |
|---|---|---|
| (Intermediate) | Structure | Name and Analytical Data |
| 104 (Example 24) | 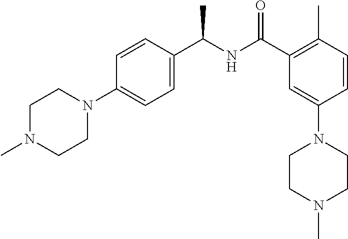 [SMILES: Cc1ccc(N2CCN(C)CC2)cc1C(=O)N[C@H](C)c1ccc(N2CCN(C)CC2)cc1] | 2-Methyl-5-(4-methylpiperazin-1-yl)-N- [(1R)-1-[4-(4-methylpiperazin-1- yl)phenyl]ethyl]benzamide ¹H NMR (500 MHz, CDCl₃) δ 7.29-7.27 (m, 2H), 7.07 (d, J = 8.4, 1H), 6.93-6.89 (m, 3H), 6.88-6.83 (m, 1H), 5.85 (br d, J = 7.9, 1H), 5.25 (quin, J = 7.1, 1H), 3.25-3.18 (m, 4H), 3.18-3.10 (m, 4H), 2.60-2.54 (m, 7H), 2.37- 2.25 (m, 10H), 1.57 (d, 3H). LC-MS (Method A): R_{T} = 2.12, m/z = 436.8 [M + H]⁺. |

### Example 105: 2-Methyl-5-(4-methylpiperazin-1-yl)-N-[(1R)-1-(3-piperazin-1-ylphenyl)ethyl]benzamide

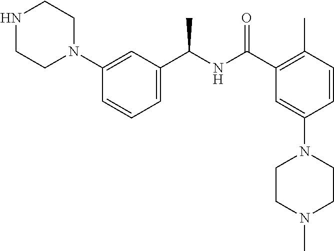 [SMILES: Cc1ccc(N2CCN(C)CC2)cc1C(=O)N[C@H](C)c1cccc(N2CCNCC2)c1]

Using General Procedure 4 with tert-butyl 4-[3-[(1R)-1-[[2-methyl-5-(4-methylpiperazin-1-yl)benzoyl]amino]ethyl]phenyl]piperazine-1-carboxylate (323 mg, 619 μmol)—prepared in a similar manner to 2-methyl-5-(4-methylpiperazin-1-yl)-N-[(1R)-1-[3-(4-methylpiperazin-1-yl)phenyl] ethyl]benzamide (Example 103)—gave 2-methyl-5-(4-methylpiperazin-1-yl)-N-[(1R)-1-(3-piperazin-1-ylphenyl)ethyl]benzamide (95 mg, 35%) as a pale yellow solid. ¹H NMR (500 MHz, DMSO-d₆) δ 8.58 (d, J=8.0, 1H), 7.15 (t, J=8.0, 1H), 7.06 (d, J=8.5, 1H), 6.98 (br s, 1H), 6.91 (dd, J=8.5, 2.5, 1H), 6.83 (d, J=2.5, 1H), 6.79-6.76 (m, 2H), 5.05 (quin, J=7.5, 1H), 3.10-3.07 (m, 4H), 3.04-3.02 (m, 4H), 2.83-2.81 (m, 4H), 2.45-2.43 (m, 4H), 2.22 (s, 3H), 2.17 (s, 3H), 1.39 (d, J=7.0, 3H). LC-MS (Method B): R_{T}=2.83, m/z=420.8 [M−H]⁻.

### Example 106: 2-Methyl-5-(4-methylpiperazin-1-yl)-N-[(1R)-1-(4-piperazin-1-ylphenyl)ethyl]benzamide

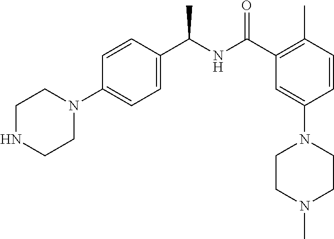 [SMILES: Cc1ccc(N2CCN(C)CC2)cc1C(=O)N[C@H](C)c1ccc(N2CCNCC2)cc1]

Using General Procedure 4 with tert-butyl 4-[4-[(1R)-1-[[2-methyl-5-(4-methylpiperazin-1-yl)benzoyl]amino]ethyl]phenyl]piperazine-1-carboxylate (100 mg, 192 μmol)—prepared in a similar manner to 2-methyl-5-(4-methylpiperazin-1-yl)-N-[(1R)-1-[3-(4-methylpiperazin-1-yl)phenyl] ethyl]benzamide (Example 104)—gave 2-methyl-5-(4-methylpiperazin-1-yl)-N-[(1R)-1-(4-piperazin-1-ylphenyl)ethyl]benzamide (24 mg, 28%) as a very pale brown foam. ¹H NMR (500 MHz, CDCl₃) δ 7.28 (d, J=8.7, 2H), 7.07 (d, J=8.4, 1H), 6.93-6.88 (m, 3H), 6.86 (dd, J=2.7, 8.3, 1H), 5.86 (br d, J=7.9, 1H), 5.30-5.22 (m, 1H), 3.19-3.12 (m, 7H), 3.12-2.98 (m, 4H), 2.60-2.54 (m, 4H), 2.35 (s, 3H), 2.31 (s, 3H), 1.57 (d, J=6.7, 3H). LC-MS (Method A): R_{T}=1.98, m/z=422.7 [M+H]⁺.

### Example 107: N-[(1R)-1-[3-[5-(Hydroxymethyl)-2-oxo-oxazolidin-3-yl]phenyl]ethyl]-2-methyl-5-(4-methylpiperazin-1-yl)benzamide

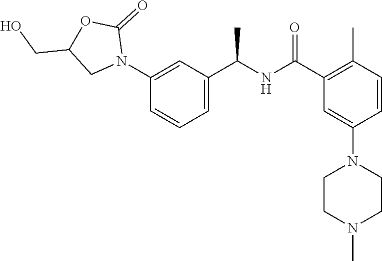 [SMILES: Cc1ccc(N2CCN(C)CC2)cc1C(=O)N[C@H](C)c1cccc(N2CC(CO)OC2=O)c1]

### Step A: N-[(1R)-1-[3-[5-[[tert-Butyl(dimethyl)silyl]oxymethyl]-2-oxo-oxazolidin-3-yl]phenyl]ethyl]-2-methyl-5-(4-methylpiperazin-1-yl)benzamide

Copper(I) Iodide (26 mg, 137 μmol) was added to a solution of N-[(1R)-1-(3-bromophenyl)ethyl]-2-methyl-5-(4-methylpiperazin-1-yl)benzamide (286 mg, 687 μmol) (Example 22), 5-[[tert-butyl(dimethyl)silyl]oxymethyl]oxazolidin-2-one (191 mg, 824 μmol), N,N′-Dimethylethylenediamine (12 mg, 137 μmol) and potassium carbonate (285 mg, 2.06 mmol) in 1,4-dioxane (20 mL) under nitrogen atmosphere and the reaction mixture was heated to 120° C. overnight. The reaction mixture was allowed to cool to RT and water (75 mL) and ethyl acetate (75 mL) were added. The phases were separated, and the aqueous phase extracted with ethyl acetate (100 mL). The combined organic phases were washed with brine (120 mL), dried (Na₂SO₄) and the solvent removed in vacuo. Purification by FCC (eluting with 0-30% MeOH in DCM) gave N-[(1R)-1-[3-[5-[[tert-butyl(dimethyl)silyl]oxymethyl]-2-oxo-oxazolidin-3-yl]phenyl]ethyl]-2-methyl-5-(4-methylpiperazin-1-yl)benzamide (327 mg, 84%) as a yellow oil. LC-MS (Method B): R_{T}=4.29, m/z=566.1 [M−H]⁻.

### Step B: N-[(1R)-1-[3-[5-(Hydroxymethyl)-2-oxo-oxazolidin-3-yl]phenyl]ethyl]-2-methyl-5-(4-methylpiperazin-1-yl)benzamide

Using General Procedure 4 with N-[(1R)-1-[3-[5-[[tert-butyl(dimethyl)silyl]oxymethyl]-2-oxo-oxazolidin-3-yl]phenyl]ethyl]-2-methyl-5-(4-methylpiperazin-1-yl)benzamide (327 mg, 577 μmol) gave N-[(1R)-1-[3-[5-(hydroxymethyl)-2-oxo-oxazolidin-3-yl]phenyl]ethyl]-2-methyl-5-(4-methylpiperazin-1-yl)benzamide (185 mg, 67%) as a white crystalline solid. ¹H NMR (500 MHz, DMSO-d₆) δ 8.71 (dd, J=8.0, 2.5, 1H), 7.69-7.67 (m, 1H), 7.47-7.45 (m, 1H), 7.38 (t, J=8.0, 1H), 7.18 (d, J=7.5, 1H), 7.09 (d, J=8.0, 1H), 6.95-6.93 (m, 2H), 5.26 (td, J=5.5, 3.5, 1H), 5.13 (quin, J=7.0, 1H), 4.74-4.72 (m, 1H), 4.12 (dt, J=14.5, 9.0, 1H), 3.89 (ddd, J=14.5, 8.5, 6.0, 1H), 3.72-3.70 (m, 1H), 3.60-3.58 (m, 1H), 3.15-3.13 (m, 4H), 2.49-2.47 (m, 4H), 2.26 (s, 3H), 2.21 (s, 3H), 1.46 (d, J=7.0, 3H). LC-MS (Method B): R_{T}=3.04, m/z=451.9 [M−H]⁻.

### Example 108: 2-Methyl-N-[(1R)-1-(1-naphthyl)ethyl]-5-piperazin-1-yl-benzamide

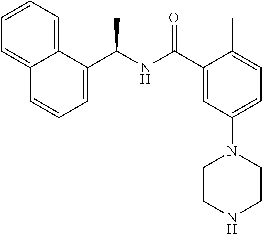 [SMILES: Cc1ccc(N2CCNCC2)cc1C(=O)N[C@H](C)c1cccc2ccccc12]

tert-Butyl 4-[4-methyl-3-[[(1R)-1-(1-naphthyl)ethyl]carbamoyl]phenyl]piperazine-1-carboxylate (Example 28) (1.55 g, 3.27 mmol) was dissolved in DCM (40 mL), 2 drops of water were added and to this was added trifluoroacetic acid (2.42 mL, 32.7 mmol) dropwise over one minute giving a clear red solution which was stirred for 4 hours at RT. The reaction was then quenched by careful addition of sat. aq. K₂CO₃. The organic layer was then separated then the aqueous extracted with DCM (2×20 mL). The combined organics were dried (Na₂SO₄), filtered and concentrated in vacuo to afford 2-methyl-N-[(1R)-1-(1-naphthyl)ethyl]-5-piperazin-1-yl-benzamide (1.09 g, 85%) as a beige foam. ¹H NMR (500 MHz, CDCl₃) δ 8.24 (d, J=8.5, 1H), 7.88 (d, J=7.9, 1H), 7.85-7.77 (m, 1H), 7.60-7.54 (m, 2H), 7.53-7.49 (m, 1H), 7.48-7.43 (m, 1H), 7.05 (d, J=8.4, 1H), 6.87-6.81 (m, 2H), 6.18-6.07 (m, 1H), 5.95 (br d, J=8.2, 1H), 3.06-2.94 (m, 8H), 2.31 (s, 3H), 1.79 (d, J=6.7, 3H). LC-MS (Method A): RT=3.14, m/z=374.6 [M+H]⁺.

### Example 109: 5-(4-Acetylpiperazin-1-yl)-2-methyl-N-[(1R)-1-(1-naphthyl)ethyl]benzamide

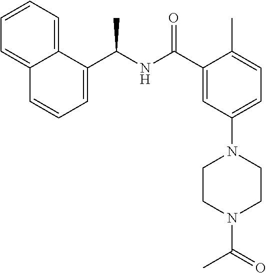 [SMILES: CC(=O)N1CCN(c2ccc(C)c(C(=O)N[C@H](C)c3cccc4ccccc34)c2)CC1]

Under an inert atmosphere, 2-methyl-N-[(1R)-1-(1-naphthyl)ethyl]-5-piperazin-1-yl-benzamide (Example 109) (84.0 mg, 225 μmol) was suspended in DCM (3 mL) and DIPEA (115 μL, 674 μmol) added. The suspension was cooled in an ice bath and acetyl chloride (24 μL, 337 μmol) added. The solution was allowed to warm to RT and stirred for 2 hours. The mixture was partitioned between DCM (30 mL) and 2M K₂CO₃ aq. (10 mL). The organic was separated, dried (Na₂SO₄), filtered and concentrated in vacuo to give the crude product. Purification was by FCC (eluting with 0-100% MeOH in DCM) to give 5-(4-acetylpiperazin-1-yl)-2-methyl-N-[(1R)-1-(1-naphthyl)ethyl]benzamide (92 mg, 94%) as a pale yellow foam. ¹H NMR (500 MHz, DMSO-d₆) δ 8.82 (d, J=7.9, 1H), 8.24 (d, J=8.4, 1H), 7.98-7.94 (m, 1H), 7.84 (d, J=8.1, 1H), 7.64-7.50 (m, 4H), 7.08 (d, J=8.5, 1H), 6.93 (dd, J=2.6, 8.4, 1H), 6.89 (d, J=2.6, 1H), 5.90 (t, J=7.3, 1H), 3.61-3.53 (m, 4H), 3.14-3.08 (m, 2H), 3.08-3.02 (m, 2H), 2.19 (s, 3H), 2.04 (s, 3H), 1.59 (d, J=6.9, 3H). LC-MS (Method A): R_{T}=3.25, m/z=414.8 [M−H]⁻.

### Example 110: 2-Methyl-5-(4-methylsulfonylpiperazin-1-yl)-N-[(1R)-1-(1-naphthyl)ethyl]benzamide

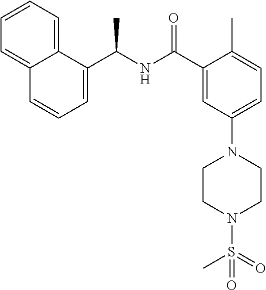 [SMILES: Cc1ccc(N2CCN(S(C)(=O)=O)CC2)cc1C(=O)N[C@H](C)c1cccc2ccccc12]

Under inert atmosphere, 2-methyl-N-[(1R)-1-(1-naphthyl)ethyl]-5-piperazin-1-yl-benzamide (Example 109) (102 mg, 273 μmol) was suspended in DCM (3 mL) and DIPEA (140 μL, 819 μmol) added. The solution was cooled in an ice bath and methanesulfonyl chloride (31.7 μL, 410 μmol) added. After 1 hour the mixture was partitioned between DCM (50 mL) and 2M K₂CO₃ aq. (10 mL). The organic was separated, dried (Na₂SO₄), filtered, and evaporated in vacuo to give crude product. Purification was by FCC (eluting with 0-20% MeOH in DCM) to give 2-methyl-5-(4-methylsulfonylpiperazin-1-yl)-N-[(1R)-1-(1-naphthyl)ethyl]benzamide (120 mg, 92%) as an off white solid. ¹H NMR (500 MHz, DMSO-d₆) δ 8.83 (d, J=7.9, 1H), 8.24 (d, J=8.4, 1H), 7.96 (d, J=7.5, 1H), 7.84 (d, J=8.1, 1H), 7.64-7.50 (m, 4H), 7.09 (d, J=8.4, 1H), 6.95 (dd, J=2.7, 8.3, 1H), 6.93-6.88 (m, 1H), 5.90 (quin, J=7.1, 1H), 3.30-3.15 (m, 8H), 2.93 (s, 3H), 2.19 (s, 3H), 1.59 (d, J=7.0, 3H). LC-MS (Method A): R_{T}=3.55, m/z=450.8 [M−H]⁻.

### Example 111: 5-[4-(2-Hydroxyethyl)piperazin-1-yl]-2-methyl-N-[(1R)-1-(1-naphthyl)ethyl]benzamide

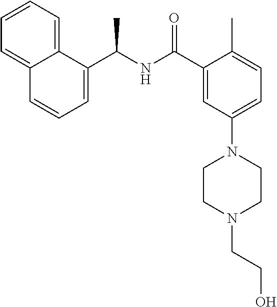 [SMILES: Cc1ccc(N2CCN(CCO)CC2)cc1C(=O)N[C@H](C)c1cccc2ccccc12]

Under an inert atmosphere, 2-methyl-N-[(1R)-1-(1-naphthyl)ethyl]-5-piperazin-1-yl-benzamide (Example 109) (175 mg, 469 μmol) and potassium carbonate (187 mg, 1.35 mmol) were suspended in ethanol (6 mL) and 2-bromoethanol (49.9 μL, 702 μmol) added. The suspension was heated at reflux overnight. Upon cooling the mixture was partitioned between ethyl acetate (50 mL) and water (20 mL). The organic was washed with brine (10 mL), dried (Na₂SO₄), filtered and evaporated under reduced pressure to give the crude product. Purification was by FCC (eluting with 0-20% 1M NH₃/MeOH in DCM) to give 5-[4-(2-hydroxyethyl)piperazin-1-yl]-2-methyl-N-[(1R)-1-(1-naphthyl)ethyl]benzamide (108 mg, 52%) as a white foam. ¹H NMR (500 MHz, DMSO-d₆) δ 8.81 (d, J=7.9, 1H), 8.24 (d, J=8.4, 1H), 7.98-7.94 (m, 1H), 7.84 (d, J=8.1, 1H), 7.64-7.50 (m, 4H), 7.05 (d, J=8.4, 1H), 6.89 (dd, J=2.6, 8.4, 1H), 6.87-6.83 (m, 1H), 5.89 (quin, J=7.2, 1H), 4.42 (t, J=5.3, 1H), 3.53 (q, J=6.2, 2H), 3.13-3.03 (m, 4H), 2.56-2.52 (m, 4H), 2.43 (t, J=6.3, 2H), 2.17 (s, 3H), 1.58 (d, J=7.0, 3H). LC-MS (Method A): R_{T}=3.38, m/z=418.6 [M+H]⁺.

### Example 112: 5-[4-(2-Hydroxyethyl)piperazin-1-yl]-2-methyl-N-[(1R)-1-[3-(1-methylpyrazol-4-yl)phenyl]ethyl]benzamide

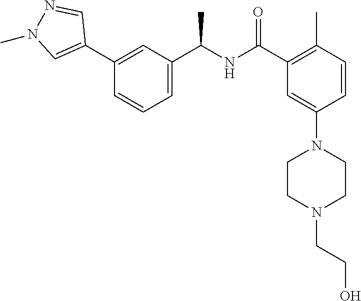 [SMILES: Cc1ccc(N2CCN(CCO)CC2)cc1C(=O)N[C@H](C)c1cccc(-c2cnn(C)c2)c1]

### Step A: 5-[4-[2-[tert-Butyl(dimethyl)silyl]oxyethyl]piperazin-1-yl]-2-methyl-N-[(1R)-1-[3-(1-methylpyrazol-4-yl)phenyl]ethyl]benzamide

2-Bromoethoxy-tert-butyldimethylsilane (41 mg, 172 μmol) was added to a solution of 2-methyl-N-[(1R)-1-[3-(1-methylpyrazol-4-yl)phenyl]ethyl]-5-piperazin-1-yl-benzamide (58 mg, 144 μmol) (Example 72) and potassium carbonate (40 mg, 287 μmol) in DMF (20 mL) and the reaction mixture heated to 70° C. overnight. The reaction mixture was allowed to cool to RT and water (75 mL) and petroleum ether (75 mL) added. The phases were separated, and the aqueous phase extracted with petroleum ether (75 mL), then ethyl acetate (75 mL). The combined organic phases were washed with brine (100 mL), dried over sodium sulfate and the solvent removed in vacuo. Purification by FCC (eluting with 0-50% ethyl acetate in petroleum ether) gave 5-[4-[2-[tert-butyl(dimethyl)silyl]oxyethyl]piperazin-1-yl]-2-methyl-N-[(1R)-1-[3-(1-methylpyrazol-4-yl)phenyl]ethyl]benzamide (80 mg, 99%) as a yellow oil. LC-MS (Method B): R_{T}=4.75, m/z=562.9 [M+H]⁺.

### Step B: 5-[4-(2-Hydroxyethyl)piperazin-1-yl]-2-methyl-N-[(1R)-1-[3-(1-methylpyrazol-4-yl)phenyl]ethyl]benzamide

Using General Procedure 4 with 5-[4-[2-[tert-butyl(dimethyl)silyl]oxyethyl]piperazin-1-yl]-2-methyl-N-[(1R)-1-[3-(1-methylpyrazol-4-yl)phenyl]ethyl]benzamide (88 mg, 157 μmol) gave 5-[4-(2-hydroxyethyl)piperazin-1-yl]-2-methyl-N-[(1R)-1-[3-(1-methylpyrazol-4-yl)phenyl]ethyl]benzamide (21 mg, 30%) as a white crystalline solid. ¹H NMR (500 MHz, DMSO-d₆) δ 8.57 (d, J=8.0, 1H), 8.04 (s, 1H), 7.76 (s, 1H), 7.54 (br s, 1H), 7.36 (d, J=7.5, 1H), 7.26 (t, J=7.5, 1H), 7.15 (d, J=7.5, 1H), 7.00 (d, J=8.5, 1H), 6.84 (dd, J=8.5, 2.5, 1H), 6.79 (d, J=2.5, 1H), 5.06 (quin, J=7.0, 1H), 3.80 (s, 3H), 3.48 (q, J=6.0, 2H), 3.04 (m, 4H), 2.49 (m, 4H), 2.37 (t, J=6.0, 2H), 2.11 (s, 3H), 1.39 (d, J=7.0, 3H). LC-MS (Method B): R_{T}=3.02, m/z=446.9 [M−H]⁻.

### Example 113: 5-[4-[2-(Dimethylamino)-2-oxo-ethyl]piperazin-1-yl]-2-methyl-N-[(1R)-1-[3-(1-methylpyrazol-4-yl)phenyl]ethyl]benzamide

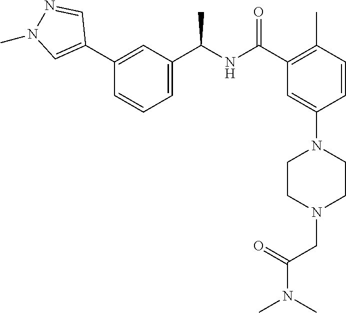 [SMILES: Cc1ccc(N2CCN(CC(=O)N(C)C)CC2)cc1C(=O)N[C@H](C)c1cccc(-c2cnn(C)c2)c1]

2-Bromo-N,N-dimethyl-acetamide (46 mg, 277 μmol) was added to a solution of 2-methyl-N-[(1R)-1-[3-(1-methylpyrazol-4-yl)phenyl]ethyl]-5-piperazin-1-yl-benzamide (93 mg, 231 μmol) (Example 72) and potassium carbonate (64 mg, 462 μmol) in DMF (20 mL) and the reaction mixture heated to 70° C. for 3 hours. The reaction mixture was allowed to cool to RT and water (75 mL) and ethyl acetate (75 mL) added. The organic phase was washed with brine (100 mL), dried over sodium sulfate and the solvent removed in vacuo. Purification by FCC (eluting with 0-30% MeOH in DCM) gave 5-[4-[2-(dimethylamino)-2-oxo-ethyl]piperazin-1-yl]-2-methyl-N-[(1R)-1-[3-(1-methylpyrazol-4-yl)phenyl]ethyl]benzamide (39 mg, 33%) as a white crystalline solid. ¹H NMR (500 MHz, DMSO-d₆) δ 8.63 (d, J=8.0, 1H), 8.10 (s, 1H), 7.82 (s, 1H), 7.60 (s, 1H), 7.42 (d, J=7.5, 1H), 7.32 (t, J=7.5, 1H), 7.22 (d, J=7.5, 1H), 7.06 (d, J=8.5, 1H), 6.91 (dd, J=8.0, 2.5, 1H), 6.86 (d, J=2.5, 1H), 5.12 (quin, J=7.0, 1H), 3.87 (s, 3H), 3.19 (br s, 2H), 3.11 (m, 4H), 3.04 (s, 3H), 2.83 (s, 3H), 2.58 (m, 4H), 2.18 (s, 3H), 1.45 (d, J=7.0, 3H). LC-MS (Method B): R_{T}=3.36, m/z=488.0 [M−H]⁻.

### Example 114: 5-[4-(2-Methoxyethyl)piperazin-1-yl]-2-methyl-N-[(1R)-1-[3-(1-methylpyrazol-4-yl)phenyl]ethyl]benzamide

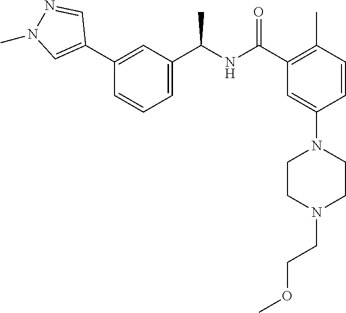 [SMILES: COCCN1CCN(c2ccc(C)c(C(=O)N[C@H](C)c3cccc(-c4cnn(C)c4)c3)c2)CC1]

1-Bromo-2-methoxy-ethane (41 mg, 297 μmol) was added to a solution of 2-methyl-N-[(1R)-1-[3-(1-methylpyrazol-4-yl)phenyl]ethyl]-5-piperazin-1-yl-benzamide (100 mg, 248 μmol) (Example 72) and potassium carbonate (69 mg, 496 μmol) in DMF (20 mL) and the reaction mixture heated to 70° C. for 3 hours. The reaction mixture was allowed to cool to RT and water (75 mL) and ethyl acetate (75 mL) added. The organic phase was washed with brine (100 mL), dried over sodium sulfate and the solvent removed in vacuo. Purification by FCC (eluting with 0-30% MeOH in DCM) gave 5-[4-(2-methoxyethyl)piperazin-1-yl]-2-methyl-N-[(1R)-1-[3-(1-methylpyrazol-4-yl)phenyl]ethyl]benzamide (41 mg, 34%) as a pale pink crystalline solid. ¹H NMR (500 MHz, DMSO-d₆) δ 8.63 (d, J=8.0, 1H), 8.10 (s, 1H), 7.82 (s, 1H), 7.59 (br s, 1H), 7.42 (d, J=7.5, 1H), 7.32 (t, J=7.5, 1H), 7.22 (d, J=7.5, 1H), 7.06 (d, J=8.0, 1H), 6.90 (dd, J=8.5, 2.5, 1H), 6.85 (d, J=2.5, 1H), 5.12 (quin, J=7.0, 1H), 3.87 (s, 3H), 3.47 (t, J=6.0, 2H), 3.25 (s, 3H), 3.10 (m, 4H), 2.55 (m, 4H), 2.53 (m, 2H), 2.18 (s, 3H), 1.45 (d, J=7.0, 3H). LC-MS (Method B): R_{T}=3.43, m/z=460.9 [M−H]⁻.

### Example 115: 5-(4-Cyclopropylpiperazin-1-yl)-2-methyl-N-[(1R)-1-(1-naphthyl)ethyl]benzamide

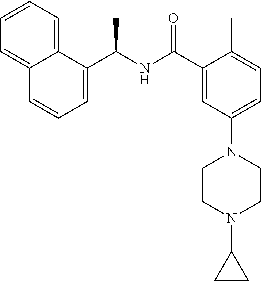 [SMILES: Cc1ccc(N2CCN(C3CC3)CC2)cc1C(=O)N[C@H](C)c1cccc2ccccc12]

### Step A: Benzyl 5-bromo-2-methyl-benzoate

5-Bromo-2-methyl-benzoic acid (34.0 g, 158 mmol) was added to DMF (180 mL) and to this was added potassium carbonate (24.0 g, 174 mmol) and benzyl bromide (18.8 mL, 158 mmol), then the mixture was stirred for 2 hours. The reaction was quenched with water (200 mL) extracted with diethyl ether (2×150 ml), dried (MgSO₄) and solvent removed in vacuo to afford a yellow liquid. Distillation at 150° C. @ 5.5 m bar afforded benzyl 5-bromo-2-methyl-benzoate (39.7 g, 80%) as a clear liquid. LC-MS (Method B): R_{T}=4.64, m/z=no mass ion visible.

### Step B: Benzyl 5-(4-cyclopropylpiperazin-1-yl)-2-methyl-benzoate

Using General Procedure 3 with 1-cyclopropylpiperazine (2.48 g, 19.6 mmol) and benzyl 5-bromo-2-methyl-benzoate (4.00 g, 13.1 mmol) at 100° C. overnight gave benzyl 5-(4-cyclopropylpiperazin-1-yl)-2-methyl-benzoate (3.47 g, 74%) as a yellow gum which solidified on standing. LC-MS (Method B): R_{T}=4.43, m/z=349.7 [M−H]⁻.

### Step C: 5-(4-Cyclopropylpiperazin-1-yl)-2-methyl-benzoic Acid

Using General Procedure 5 with benzyl 5-(4-cyclopropylpiperazin-1-yl)-2-methyl-benzoate (3.80 g, 10.8 mmol) and palladium, 10% on activated carbon powder (115 mg, 1.1 mmol) gave 5-(4-cyclopropylpiperazin-1-yl)-2-methyl-benzoic acid (2.60 g, 92%) as a white solid. LC-MS (Method B): R_{T}=0.35, m/z=not visible.

### Step D: 5-(4-Cyclopropylpiperazin-1-yl)-2-methyl-N-[(1R)-1-(1-naphthyl)ethyl]benzamide

Using General Procedure 1 with (1R)-1-(1-naphthyl)ethanamine (70 mg, 408 μmol) and 5-(4-cyclopropylpiperazin-1-yl)-2-methyl-benzoic acid (117 mg, 449 μmol) gave 5-(4-cyclopropylpiperazin-1-yl)-2-methyl-N-[(1R)-1-(1-naphthyl)ethyl]benzamide (69 mg, 40%) as a white solid. ¹H NMR (500 MHz, CDCl₃) δ 8.24 (d, J=8.2, 1H), 7.88 (d, J=8.2, 1H), 7.86-7.77 (m, 1H), 7.59-7.44 (m, 4H), 7.04 (d, J=8.5, 1H), 6.88-6.80 (m, 2H), 6.16-6.08 (m, 1H), 5.92 (br d, J=8.2, 1H), 3.05 (dd, J=3.7, 6.1, 4H), 2.76-2.68 (m, 4H), 2.60 (s, 3H), 1.79 (d, J=6.7, 3H), 1.68-1.60 (m, 1H), 0.49-0.41 (m, 4H). LC-MS (Method B): R_{T}=3.90, m/z=412.8 [M−H]⁻.

### Further Examples

The following examples were prepared in a similar manner to 5-(4-cyclopropylpiperazin-1-yl)-2-methyl-N-[(1R)-1-(1-naphthyl)ethyl]benzamide (Example 115), using the required commercially available secondary amine in Step B, and the required commercially available primary amine in Step D. Examples which did not provide solid material after Step D were treated with 2N HCl in diethyl ether, then concentrated and triturated with an appropriate solvent to give product as a hydrochloride salt.

| Example | Structure | Name and Analytical Data |
|---|---|---|
| 116 | 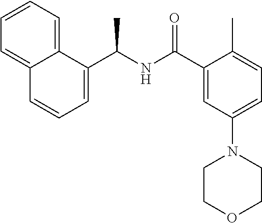 [SMILES: Cc1ccc(N2CCOCC2)cc1C(=O)N[C@H](C)c1cccc2ccccc12] | 2-Methyl-5-morpholino-N-[(1R)-1-(1- naphthyl)ethyl]benzamide ¹H NMR (500 MHz, CDCl₃) δ 8.24 (d, J = 8.2, 1H), 7.88 (d, J = 7.9, 1H), 7.85-7.79 (m, 1H), 7.60-75.4 (m, 2H), 7.54-7.44 (m, 2H), 7.06 (d, J = 8.2, 1H), 6.86-6.79 (m, 2H), 6.13 (quin, J = 7.1, 1H), 5.92 (br d, J = 7.9, 1H), 3.80 (t, J = 4.7, 4H), 3.09-2.99 (m, 4H), 2.31 (s, 3H), 1.80 (d, J = 7.1, 3H). LC-MS (Method B): R_{T} = 3.61, m/z = 373.7 [M − H]⁻. |
| 117 | 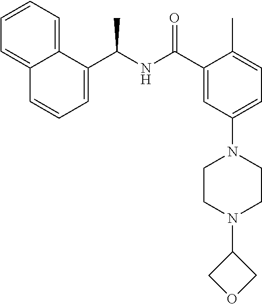 [SMILES: Cc1ccc(N2CCN(C3COC3)CC2)cc1C(=O)N[C@H](C)c1cccc2ccccc12] | 2-Methyl-N-[(1R)-1-(1-naphthyl)ethyl]-5-[4- (oxetan-3-yl)piperazin-1-yl]benzamide ¹H NMR (500 MHz, CDCl₃) δ 8.24 (d, J = 8.5, 1H), 7.88 (d, J = 7.6, 1H), 7.86-7.77 (m, 1H), 7.61-7.54 (m, 2H), 7.54-7.44 (m, 2H), 7.05 (d, J = 8.2, 1H), 6.89-6.80 (m, 2H), 6.17-6.08 (m, 1H), 5.91 (br d, J = 8.2, 1H), 4.67 (d, J = 6.7, 2H), 4.65-4.61 (m, 2H), 3.53 (quin, J = 6.4, 1H), 3.12 (dd, J = 3.8, 6.3 4H), 2.49-2.40 (m, 4H), 2.30 (s, 3H), 1.79 (d, J = 6.7, 3H). LC-MS (Method B): R_{T} = 3.30, m/z = 428.8 [M − H]⁻. |
| 118 | 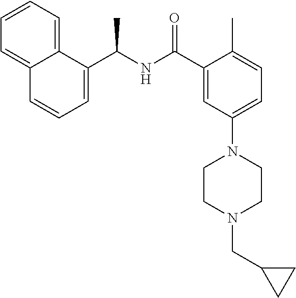 [SMILES: Cc1ccc(N2CCN(CC3CC3)CC2)cc1C(=O)N[C@H](C)c1cccc2ccccc12] | 5-[4-(Cyclopropylmethyl)piperazin-1-yl]-2- methyl-N-[(1R)-1-(1- naphthyl)ethyl]benzamide ¹H NMR (500 MHz, CDCl₃) δ 8.24 (d, J = 8.2, 1H), 7.88 (d, J = 7.9, 1H), 7.81 (d, J = 8.2, 1H), 7.60-7.44 (m, 4H), 7.04 (d, J = 8.5, 1H), 6.88- 6.82 (m, 2H), 6.12 (quin, J = 7.2, 1H), 5.93 (br d, J = 8.2, 1H), 3.12 (dd, J = 3.5, 6.0, 4H), 2.64 (t, J = 4.9, 4H), 2.33-2.28 (m, 5H), 1.79 (d, J = 7.0, 3H), 0.94-0.82 (m, 1H), 0.56-0.51 (m, 2H), 0.16- 0.08 (m, 2H). LC-MS (Method B): R_{T} = 3.96, m/z = 426.8 [M − H]⁻. |
| 119 | 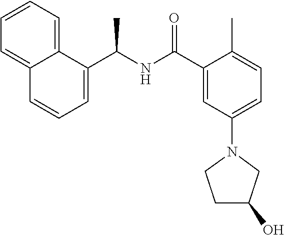 [SMILES: Cc1ccc(N2CC[C@H](O)C2)cc1C(=O)N[C@H](C)c1cccc2ccccc12] | 5-[(3S)-3-Hydroxypyrrolidin-1-yl]-2-methyl- N-[(1R)-1-(1-naphthyl)ethyl]benzamide ¹H NMR (500 MHz, CDCl₃) δ 8.25 (d, J = 8.5, 1H), 7.87 (d, J = 8.2, 1H), 7.86-7.76 (m, 1H), 7.60-7.42 (m, 4H), 7.00 (d, J = 8.2, 1H), 6.54- 6.43 (m, 2H), 6.12 (quin, J = 7.2, 1H), 6.02-5.88 (m, 1H), 4.54 (br s, 1H), 3.48 (s, 1H), 3.45-3.34 (m, 2H), 3.28-3.20 (m, 1H), 3.20-3.14 (m, 1H), 2.28 (s, 3H), 2.26-2.08 (m, 1H), 2.06-1.95 (m, 1H), 1.85-1.71 (d, J = 7.1, 3H). LC-MS (Method B): R_{T} = 3.77, m/z = 373.7 [M − H]⁺. |
| 120 | 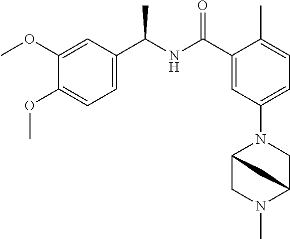 [SMILES: COc1ccc([C@@H](C)NC(=O)c2cc(N3C[C@H]4C[C@@H]3CN4C)ccc2C)cc1OC] | 2-Methyl-5-[(1R,4R)-5-methyl-2,5- diazabicyclo[2.2.1]heptan-2-yl]-N-[(1R)-1-(1- naphthyl)ethyl]benzamide ¹H NMR (500 MHz, CDCl₃) δ 7.01 (d, J = 8.5, 1H), 6.95-6.90 (m, 2H), 6.87-6.82 (m, 1H), 6.56- 6.48 (m, 2H), 5.96 (br d, J 7.9, 1H), 5.28 (quin, J = 7.2, 1H), 4.17-4.09 (m, 1H), 3.89 (s, 3H), 3.87 (s, 3H), 3.50-3.42 (m, 1H), 3.34 (dd, J = 2.0, 9.0, 1H), 3.30-3.23 (m, 1H), 2.93-2.83 (m, 1H), 2.59 (d, J = 9.5, 1H), 2.34 (s, 3H), 2.28 (s, 3H), 1.96 (br d, J = 9.5, 1H), 1.88-1.82 (m, 1H), 1.58 (d, J = 6.7, 3H). LC-MS (Method B): R_{T} = 3.30 m/z = 409.8 [M − H]⁻. |
| 121 | 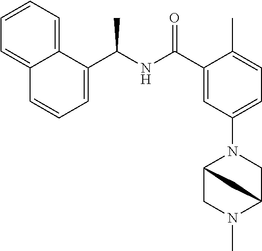 [SMILES: Cc1ccc(N2C[C@H]3C[C@@H]2CN3C)cc1C(=O)N[C@H](C)c1cccc2ccccc12] | 2-Methyl-5-[(1R,4R)-5-methyl-2,5- diazabicyclo[2.2.1]heptan-2-yl]-N-[(1R)-1- (naphthalen-1-yl)ethyl]benzamide ¹H NMR (500 MHz, CDCl₃) δ 8.24 (d, J = 8.5, 1H), 7.88 (br d, J = 7.9, 1H), 7.84-7.78 (m, 1H), 7.62-7.42 (m, 4H), 6.97 (br d, J = 7.9, 1H), 6.51- 6.39 (m, 2H), 6.15-6.05 (m, 1H), 6.05-5.99 (m, 1H), 4.07 (s, 1H), 3.50 (br s, 1H), 3.35-3.27 (m, 1H), 3.27-3.17 (m, 1H), 2.92-2.84 (m, 1H), 2.63 (br d, J = 9.8, 1H), 2.35 (s, 3H), 2.28-2.26 (m, 3H), 1.98-1.90 (m, 1H), 1.85 (br d, J = 9.8, 1H), 1.78 (d, J = 6.4, 3H). LC-MS (Method B): R_{T} = 3.95 m/z = 398.8 [M − H]⁻. |
| 122 | 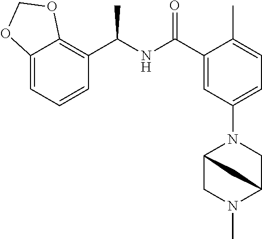 [SMILES: Cc1ccc(N2C[C@H]3C[C@@H]2CN3C)cc1C(=O)N[C@H](C)c1cccc2c1OCO2] | N-[(1R)-1-(1,3-benzodioxol-4-yl)ethyl]-2- methyl-5-[(1R,4R)-5-methyl-2,5- diazabicyclo[2.2.1]heptan-2-yl]benzamide ¹H NMR (500 MHz, DMSO-d₆) δ 8.52 (d, J = 8.5, 1H), 7.00-6.98 (m, 2H), 6.86-6.84 (m, 2H), 6.55 (dd, J = 8.5, 2.5, 1H), 6.46 (d, J = 2.5, 1H), 5.99 (m, 2H), 5.03 (quin, J = 7.0, 1H), 4.28 (br s, 1H), 3.49 (br s, 1H), 3.16 (br d, J = 9.0, 1H), 2.80 (br d, J = 9.0, 1H), 2.30 (br s, 1H), 2.15 (s, 3H), 1.90- 1.89 (m, 1H), 1.77 (br d, J = 9.0, 1H), 1.38 (d, J = 7.0, 3H). LC-MS (Method B): R_{T} = 3.55, m/z = 392.8 [M − H]⁻. |
| 123 | 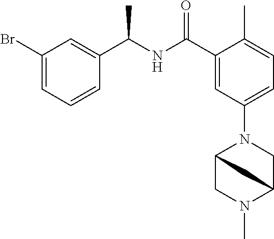 [SMILES: Cc1ccc(N2C[C@H]3C[C@@H]2CN3C)cc1C(=O)N[C@H](C)c1cccc(Br)c1] | N-[(1R)-1-(3-bromophenyl)ethyl]-2-methyl-5- [(1R,4R)-5-methyl-2,5- diazabicyclo[2.2.1]heptan-2-yl]benzamide ¹H NMR (500 MHz, DMSO-d₆) δ 8.65 (d, J = 8.0, 1H), 7.60 (br s, 1H), 7.44 (d, J = 8.0, 1H), 7.40 (d, J = 7.5, 1H), 7.32 (t, J = 8.0, 1H), 7.0 (d, J = 8.5, 1H), 6.55 (dd, J = 8.5, 2.5, 1H), 6.48 (d, J = 2.5, 1H), 5.08 (quin, J = 7.0, 1H), 4.26 (s, 1H), 3.40 (s, 1H), 3.30 (dd, J = 9.0, 2.0, 1H), 3.14 (d, J = 9.0, 1H), 2.77 (dd, J = 9.5, 2.0, 1H), 2.47 (d, J = 9.5, 1H), 2.24 (s, 3H), 2.13 (s, 3H), 1.85 (br d, J = 9.0, 1H), 1.74 (br d, J = 9.0, 1H), 1.41 (d, J = 7.0, 3H). LC-MS (Method B): R_{T} = 3.91, m/z = 426.7/42.7 [M − H]⁻. |
| 124 | 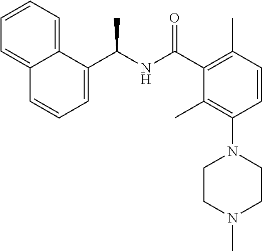 [SMILES: Cc1ccc(N2CCN(C)CC2)c(C)c1C(=O)N[C@H](C)c1cccc2ccccc12] | 2,6-Dimethyl-3-(4-methylpiperazin-1-yl)-N- [(1R)-1-(1-naphthyl)ethyl]benzamide ¹H NMR (500 MHz, CDCl₃) δ 8.31 (d, J = 8.5, 1H), 7.88 (d, J = 7.9, 1H), 7.85-7.78 (m, 2H), 7.63-7.50 (m, 1H), 7.48-7.42 (m, 1H), 7.33- 7.28 (m, 1H), 6.92 (d, J = 8.2, 1H), 6.88-6.80 (m, 1H), 6.23-6.17 (m, 1H), 5.84 (br d, J = 8.2, 1H), 3.17-2.82 (m, 8H), 2.66-2.60 (m, 3H), 2.27- 2.08 (m, 6H), 1.82 (d, J = 6.7, 3H). LC- MS (Method B): R_{T} = 3.83, m/z = 400.7 [M − H]⁻. |
| 125 | 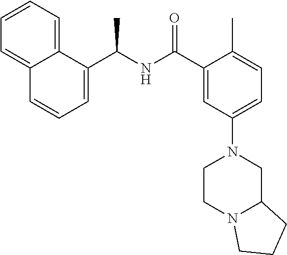 [SMILES: Cc1ccc(N2CCN3CCCC3C2)cc1C(=O)N[C@H](C)c1cccc2ccccc12] | 5-(3,4,6,7,8,8a-Hexahydro-1H-pyrrolo[1,2- a]pyrazin-2-yl)-2-methyl-N-[(1R)-1-(1- naphthyl)ethyl]benzamide hydrochloride salt ¹H NMR (500 MHz, D₂O) δ 8.14 (d, J = 8.5, 1H), 7.92 (d, J = 7.9, 1H), 7.83 (d, J = 8.2, 1H), 7.61- 7.44 (m, 4H), 7.15 (d, J = 8.2, 1H), 7.02-6.95 (m, 1H), 6.81 (br s, 1H), 5.87-5.79 (m, 1H), 3.83 (brs, 1H), 3.75-3.55 (m, 2H), 3.39-3.13 (m, 4H), 3.01-2.70 (m, 3H), 2.28-1.98 (m, 6H), 1.63 (d, J = 7.0, 3H). LC-MS (Method B): R_{T} = 3.82, m/z = 412.7 [M − H]⁻. |
| 126 | 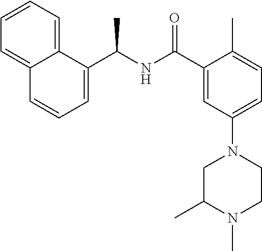 [SMILES: Cc1ccc(N2CCN(C)C(C)C2)cc1C(=O)N[C@H](C)c1cccc2ccccc12] | 5-(3,4-Dimethylpiperazin-1-yl)-2-methyl-N- [(1R)-1-(1-naphthyl)ethyl]benzamide ¹H NMR (500 MHz, CDCl₃) δ 8.23 (br d, J = 7.0, 1H), 7.88 (dd, J = 4.1, 7.8, 1H), 7.85-7.79 (m, 1H), 7.68-7.54 (m, 2H), 7.54-7.39 (m, 2H), 7.04 (dd, J = 2.6, 8.4, 1H), 6.81 (t, J = 2.7, 1H), 6.79- 6.71 (m, 1H), 6.28 (br d, J = 7.6, 1H), 6.16 (br d, J = 7.3, 1H), 3.35 (br d, J = 12.5, 2H), 3.26-3.07 (m, 1H), 3.03-2.87 (m, 1H), 2.86-2.64 (m, 3H), 2.61 (s, 3H), 2.52 (s, 3H), 2.29 (s, 3H), 1.80 (d, J = 6.7, 3H). LC-MS (Method B): R_{T} = 3.66, m/z = 400.7 [M − H]⁻. |
| 127 | 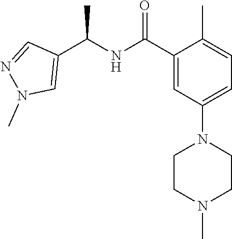 [SMILES: Cc1ccc(N2CCN(C)CC2)cc1C(=O)N[C@H](C)c1cnn(C)c1] | 2-Methyl-5-(4-methylpiperazin-1-yl)-N-[(1R)- 1-(1-methylpyrazol-4-yl)ethyl]benzamide ¹H NMR (500 MHz, DMSO-d₆) δ 8.39 (d, J = 8.4, 1H), 7.58 (s, 1H), 7.36 (s, 1H), 7.08-7.02 (m, 1H), 6.91-6.86 (m, 1H), 6.82 (s, 1H), 5.13-5.03 (m, 1H), 3.79 (s, 3H), 3.13-3.05 (m, 4H), 2.47- 2.41 (m, 4H), 2.22 (s, 3H), 2.20 (s, 3H), 1.40 (d, J = 6.8, 3H). LC-MS (Method A): R_{T} = 2.18, m/z = 364.7 [M + H]⁺. |

### Example 128: 5-(1,4-Diazepan-1-yl)-2-methyl-N-[(1R)-1-(1-naphthyl)ethyl]benzamide

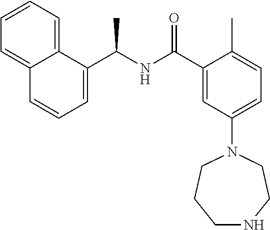 [SMILES: Cc1ccc(N2CCCNCC2)cc1C(=O)N[C@H](C)c1cccc2ccccc12]

tert-Butyl 4-[4-methyl-3-[[(1R)-1-(1-naphthyl)ethyl]carbamoyl]phenyl]-1,4-diazepane-1-carboxylate (790 mg, 1.62 mmol)—prepared in a similar manner to 5-(4-cyclopropylpiperazin-1-yl)-2-methyl-N-[(1R)-1-(1-naphthyl)ethyl]benzamide (Example 116)—was added to 6.0 HCl in propan-2-ol (10 mL) and stirred for 1 hour to afford a yellow solution. The solution was diluted with water (40 mL) and extracted with diethyl ether (50 mL). The aqueous layer was then basified to pH 11 with solid NaOH, extracted with diethyl ether (2×75 mL), dried (MgSO₄), filtered and the solvent removed in vacuo to afford a solid. This was stirred in diethyl ether (20 ml) for 1 hour to afford a solid which was filtered to give 5-(1,4-diazepan-1-yl)-2-methyl-N-[(1R)-1-(1-naphthyl)ethyl]benzamide (458 mg, 73%) as a white solid. ¹H NMR (500 MHz, CDCl₃) δ 8.24 (d, J=8.5, 1H), 7.87 (d, J=7.6, 1H), 7.81 (d, J=8.2, 1H), 7.59-7.53 (m, 2H), 7.53-7.44 (m, 2H), 6.98 (d, J=8.2, 1H), 6.63-6.57 (m, 2H), 6.11 (quin, J=7.1, 1H), 5.99 (br d, J=7.9, 1H), 3.52-3.45 (m, 4H), 3.38-3.15 (m, 1H), 2.99 (t, J=5.2, 2H), 2.86-2.80 (m, 2H), 2.26 (s, 3H), 1.96-1.84 (m, 2H), 1.79 (d, J=7.1, 3H). LC-MS (Method B): RT=4.22, m/z=386.8 [M−H]⁻.

### Example 129: 2-Methyl-N-[(1R)-1-(1-naphthyl)ethyl]-5-[(3R)-3-(1-piperidyl)pyrrolidin-1-yl]benzamide

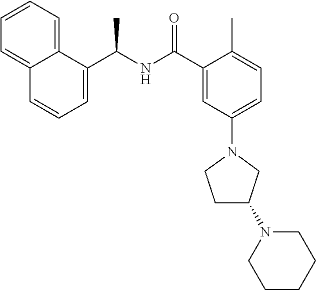 [SMILES: Cc1ccc(N2CC[C@@H](N3CCCCC3)C2)cc1C(=O)N[C@H](C)c1cccc2ccccc12]

### Step A: [(3S)-1-[4-Methyl-3-[[(1R)-1-(1-naphthyl)ethyl]carbamoyl]phenyl]pyrrolidin-3-yl]methanesulfonate

5-[(3S)-3-Hydroxypyrrolidin-1-yl]-2-methyl-N-[(1R)-1-(1-naphthyl)ethyl]benzamide (Example 120) (282 mg, 753 μmol) was dissolved in DCM (30 mL). To this was added triethylamine (136 μL, 979 μmol) and then methanesulfonyl chloride (64 μL, 828 μmol) and the mixture was stirred for 1.5 hours. The mixture was quenched with sat aq. NH₄Cl (10 mL) and water (30 mL), extracted with DCM (50 mL), dried (Na₂SO₄) and solvent removed in vacuo to afford a yellow solid. The solid was triturated with diethyl ether to afford a solid which was filtered and dried to give [(3S)-1-[4-methyl-3-[[(1R)-1-(1-naphthyl)ethyl]carbamoyl]phenyl]pyrrolidin-3-yl] methanesulfonate (290 mg, 85%) as a white solid. This was used directly in Step B.

Step B: [(3S)-1-[4-Methyl-3-[[(1R)-1-(1-naphthyl)ethyl]carbamoyl]phenyl]pyrrolidin-3-yl]methanesulfonate (100 mg, 220 μmol) in piperidine (65 μL, 662 μmol) was heated at 80° C. for 6 hours. The mixture was diluted with water (30 mL) and stirred to afford a brown solid which was filtered. This was further purified by FCC (eluting with ethyl acetate then 5% MeOH in ethyl acetate) to afford a yellow foam which was triturated with diethyl ether to afford a solid which was filtered to give 2-methyl-N-[(1R)-1-(1-naphthyl)ethyl]-5-[(3R)-3-(1-piperidyl)pyrrolidin-1-yl]benzamide (22 mg, 22%) as a yellow solid. ¹H NMR (500 MHz, CDCl₃) δ 8.24 (d, J=8.5, 1H), 7.87 (d, J=8.2, 1H), 7.81 (d, J=8.2, 1H), 7.59-7.53 (m, 2H), 7.53-7.44 (m, 2H), 6.99 (d, J=8.5, 1H), 6.47-6.43 (m, 2H), 6.11 (quin, J=7.1, 1H), 5.93 (br d, J=8.2, 1H), 3.41-3.29 (m, 2H), 3.21 (dt, J=7.2, 9.4, 1H), 3.05 (br t, J=8.4, 1H), 2.96-2.80 (m, 1H), 2.56-2.34 (m, 4H), 2.28 (s, 3H), 2.25-2.11 (m, 1H), 1.95-1.82 (m, 1H), 1.82-1.76 (m, 3H), 1.69-1.60 (m, 4H), 1.54-1.41 (m, 2H). LC-MS (Method B): R_{T}=4.32, m/z=440.8 [M−H]⁺.

### Example 130: 2-Chloro-5-(4-methylpiperazin-1-yl)-N-[(1R)-1-(1-naphthyl)ethyl]benzamide

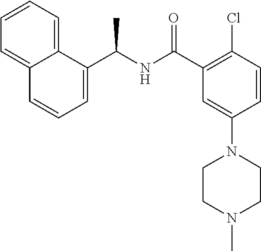 [SMILES: C[C@@H](NC(=O)c1cc(N2CCN(C)CC2)ccc1Cl)c1cccc2ccccc12]

Using General Procedure 3 with 1-methylpiperazine (51 mg, 505 μmol, 56.0 μL) and 2-chloro-5-iodo-N-[(1R)-1-(1-naphthyl)ethyl]benzamide (200 mg, 460 μmol)—prepared in a similar manner to N-[(1R)-1-(1-naphthyl)ethyl]-3-(1-piperidyl)benzamide (Example 1)—at 100° C. overnight gave 2-chloro-5-(4-methylpiperazin-1-yl)-N-[(1R)-1-(1-naphthyl)ethyl]benzamide (48 mg, 26%) as an off white solid. ¹H NMR (500 MHz, CDCl₃) δ 8.22 (d, J=8.2, 1H), 7.87 (d, J=7.9, 1H), 7.81 (d, J=7.9, 1H), 7.60-7.55 (m, 2H), 7.54-7.43 (m, 2H), 7.24-7.15 (m, 2H), 6.85 (dd, J=3.2, 9.0, 1H), 6.55 (br d, J=7.6, 1H), 6.12 (quin, J=6.7, 1H), 3.23-3.13 (m, 4H), 2.58-2.48 (m, 4H), 2.33 (s, 3H), 1.80 (d, J=6.7, 3H). LC-MS (Method B): R_{T}=3.60, m/z=406.7 [M−H]⁻.

### Further Examples

The following examples were prepared in a similar manner to 2-chloro-5-(4-methylpiperazin-1-yl)-N-[(1R)-1-(1-naphthyl)ethyl]benzamide (Example 130), using the required commercially available secondary amine.

| Example | Structure | Name and Analytical Data |
|---|---|---|
| 131 | 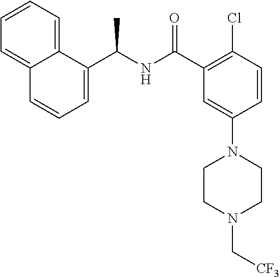 [SMILES: C[C@@H](NC(=O)c1cc(N2CCN(CC(F)(F)F)CC2)ccc1Cl)c1cccc2ccccc12] | 2-Chloro-N-[(1R)-1-(1-naphthyl)ethyl]-5-[4- (2,2,2-trifluoroethyl)piperazin-1-yl]benzamide ¹H NMR (500 MHz, CDCl₃) δ 8.22 (d, J = 8.5, 1H), 7.88 (d, J = 7.6, 1H), 7.81 (d, J = 8.2, 1H), 7.61-7.55 (m, 2H), 7.53-7.45 (m, 2H), 7.22-7.18 (m, 2H), 6.84 (dd, J = 3.1, 8.5, 1H), 6.54 (br d, J = 8.2, 1H), 6.12 (quin, J = 7.1, 1H), 3.22-3.14 (m, 4H), 3.02 (q, J = 9.7, 2H), 2.83-2.76 (m, 4H), 1.80 (d, J = 7.0, 3H). LC-MS (Method B): R_{T} = 4.25, m/z = 474.8 [M − H]⁻. |
| 132 | 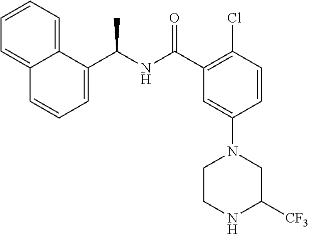 [SMILES: C[C@@H](NC(=O)c1cc(N2CCNC(C(F)(F)F)C2)ccc1Cl)c1cccc2ccccc12] | 2-Chloro-N-[(1R)-1-(1-naphthyl)ethyl]-5-[3- (trifluoromethyl)piperazin-1-yl]benzamide ¹H NMR (500 MHz, CDCl₃) δ 8.22 (d, J = 8.5, 1H), 7.88 (d, J = 7.9, 1H), 7.81 (d, J = 8.2, 1H), 7.61-7.55 (m, 2H), 7.55-7.44 (m, 2H), 7.25-7.21 (m, 2H), 6.87 (dd, J = 3.1, 8.9, 1H), 6.56 (br d, J = 7.0, 1H), 6.13 (quin, J = 7.0, 1H), 3.71-3.55 (m, 1H), 3.49- 3.40 (m, 2H), 3.22-3.14 (m, 1H), 2.97 (dt, J = 3.1, 11.4, 1H), 2.84-2.75 (m, 2H), 1.81 (d, J = 7.0, 3H). LC-MS (Method B): R_{T} = 3.84, m/z = 460.7 [M − H]⁻. |
| 133 | 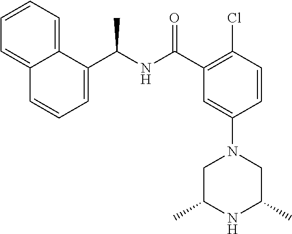 [SMILES: C[C@@H]1CN(c2ccc(Cl)c(C(=O)N[C@H](C)c3cccc4ccccc34)c2)C[C@H](C)N1] | 2-Chloro-5-[(3S,5R)-3,5-dimethylpiperazin-1- yl]-N-[(1R)-1-(1-naphthyl)ethyl]benzamide ¹H NMR (500 MHz, CDCl₃) δ 8.22 (d, J = 8.4, 1H), 7.87 (d, J 8.1, 1H), 7.84-7.78 (m, 1H), 7.61-7.54 (m, 2H), 7.54-7.44 (m, 2H), 7.22-7.17 (m, 2H), 6.84 (dd, J = 3.1, 8.9, 1H), 6.59 (br d, J = 7.8, 1H), 6.12 (quin, J = 7.1, 1H), 3.47 (br d, J = 11.4, 2H), 2.98 (ttd, J = 3.3, 6.3, 9.7, 2H), 2.32-2.23 (m, 2H), 1.80 (d, J = 6.9, 3H), 1.12 (d, J = 6.3, 6H). LC-MS (Method A): R_{T} = 3.96, m/z = 422.6 [M + H]⁺. |

### Example 134: 2-Chloro-N-[(1R)-1-(1-naphthyl)ethyl]-5-piperazin-1-yl-benzamide

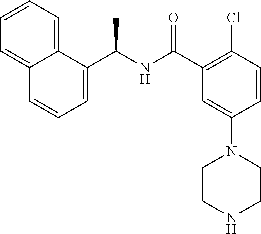 [SMILES: C[C@@H](NC(=O)c1cc(N2CCNCC2)ccc1Cl)c1cccc2ccccc12]

tert-Butyl 4-[4-chloro-3-[[(1R)-1-(1-naphthyl)ethyl]carbamoyl]phenyl]piperazine-1-carboxylate (1.32 g, 2.67 mmol)—prepared in a similar manner to 2-chloro-5-(4-methylpiperazin-1-yl)-N-[(1R)-1-(1-naphthyl)ethyl]benzamide (Example 130)—was added to 6.0 N HCl in propan-2-ol (2.67 mmol) and stirred for 1 hour to afford a cloudy solution. The mixture was evaporated to afford a yellow foam. This was dissolved in water (75 ml), extracted with diethyl ether (100 mL), the aqueous was then basified with solid NaOH. The mixture was extracted with diethyl ether (2×100 mL), dried (MgSO₄) and evaporated to afford 2-chloro-N-[(1R)-1-(1-naphthyl)ethyl]-5-piperazin-1-yl-benzamide (650 mg, 61%) as an off white foam. ¹H NMR (500 MHz, CDCl₃) δ 8.22 (d, J=8.5, 1H), 7.87 (d, J=7.9, 1H), 7.81 (d, J=8.2, 1H), 7.60-7.55 (m, 2H), 7.53-7.45 (m, 2H), 7.22-7.18 (m, 2H), 6.86 (d, J=3.1, 1H), 6.84 (d, J=3.1, 1H), 6.55 (br d, J=7.9, 1H), 6.13 (quin, J=7.1, 1H), 3.15-3.11 (m, 4H), 3.04-2.98 (m, 4H), 1.85-1.75 (m, 3H). LC-MS (Method B): R_{T}=3.61, m/z=392.7 [M−H]⁻.

### Further Examples

The following examples were prepared in a similar manner to 2-chloro-N-[(1R)-1-(1-naphthyl)ethyl]-5-piperazin-1-yl-benzamide (Example 134).

| Example | Structure | Name and Analytical Data |
|---|---|---|
| 135 | 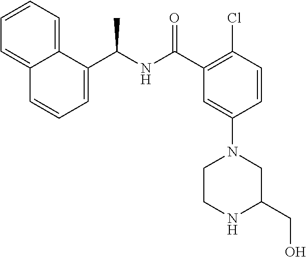 [SMILES: C[C@@H](NC(=O)c1cc(N2CCNC(CO)C2)ccc1Cl)c1cccc2ccccc12] | 2-Chloro-5-[3-(hydroxymethyl)piperazin-1-yl]- N-[(1R)-1-(1-naphthyl)ethyl]benzamide ¹H NMR (500 MHz, DMSO-d₆) δ 9.34 (br d, J = 8.9, 1H), 9.05 (br d, J = 7.9, 2H), 8.23 (d, J = 8.5, 1H), 7.96 (d, J = 7.3, 1H), 7.85 (d, J = 8.2, 1H), 7.66 (d, J = 7.0, 1H), 7.62-7.58 (m, 1H), 7.58-7.49 (m, 2H), 7.34 (d, J = 8.9, 1H), 7.06 (dd, J = 3.1, 8.9, 1H), 6.98 (d, J = 3.1, 1H), 5.88 (quin, J = 7.1, 1H), 3.85-3.69 (m, 3H), 3.69-3.62 (m, 1H), 3.41-3.27 (m, 2H), 3.18-2.99 (m, 2H), 2.95-2.83 (m, 1H), 1.59 (d, J = 7.0, 3H). LC-MS (Method B): R_{T} = 3.19, m/z = 422.7 [M − H]⁻. |
| 136 | 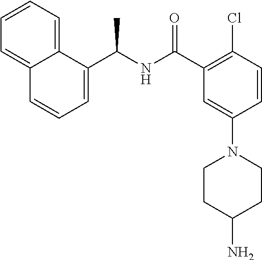 [SMILES: C[C@@H](NC(=O)c1cc(N2CCC(N)CC2)ccc1Cl)c1cccc2ccccc12] | 5-(4-Amino-1-piperidyl)-2-chloro-N-[(1R)-1-(1- naphthyl)ethyl]benzamide ¹H NMR (500 MHz, CDCl₃) δ 8.22 (d, J = 8.5, 1H), 7.87 (d, J = 8.1, 1H), 7.81 (d, J = 8.2, 1H), 7.61-7.54 (m, 2H), 7.54-7.44 (m, 2H), 7.20 (d, J = 3.1, 1H), 7.19-7.15 (m, 1H), 6.85 (dd, J = 3.1, 8.9, 1H), 6.54 (br d, J = 7.8, 1H), 6.12 (quin, J = 7.1, 1H), 3.61 (br d, J = 12.7, 2H), 2.85-2.71 (m, 3H), 1.90-1.84 (m, 2H), 1.80 (d, J = 6.9, 3H), 1.47-1.37 (m, 2H). LC- MS (Method C): R_{T} = 1.21, m/z = 408.3 [M + H]⁺. |

### Example 137: 3-[2-(4-Methylpiperazin-1-yl)pyrimidin-4-yl]-N-[(1R)-1-(1-naphthyl)ethyl]benzamide

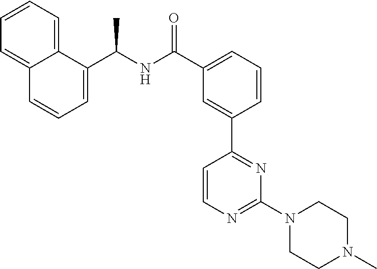 [SMILES: C[C@@H](NC(=O)c1cccc(-c2ccnc(N3CCN(C)CC3)n2)c1)c1cccc2ccccc12]

3-(2-Chloropyrimidin-4-yl)-N-[(1R)-1-(1-naphthyl)ethyl]benzamide (115 mg, 297 μmol)—prepared in a similar manner to N-[(1R)-1-(1-naphthyl)ethyl]-3-(1-piperidyl)benzamide (Example 1)—was added to DMF (10 mL) to this as added N-methylpiperazine (164 μL, 1.48 mmol) and the mixture was stirred at 100° C. for 1 hour. The mixture was quenched with water to afford a solid which was filtered. The solid was dissolved in DCM/MeOH and the DCM was removed to afford a solid, this was diluted with more MeOH and filtered to afford 3-[2-(4-methylpiperazin-1-yl)pyrimidin-4-yl]-N-[(1R)-1-(1-naphthyl)ethyl]benzamide (42 mg, 32%) as a white solid. ¹H NMR (500 MHz, CDCl₃) δ 8.43 (s, 1H), 8.40-8.34 (m, 1H), 8.19 (br d, J=8.2, 1H), 8.17-8.13 (m, 1H), 7.89 (d, J=7.9, 1H), 7.86-7.82 (m, 1H), 7.81-7.72 (m, 1H), 7.62 (d, J=7.0, 1H), 7.58-7.46 (m, 4H), 6.95 (d, J=5.2, 1H), 6.40 (br d, J=7.6, 1H), 6.15 (quin, J=6.9, 1H), 3.90 (br s, 4H), 2.47 (br s, 4H), 2.35 (s, 3H), 1.82 (d, J=6.7, 3H). LC-MS (Method B): R_{T}=3.93, m/z=450.8 [M−H]⁻.

### Further Examples

The following examples were prepared in a similar manner to 3-[2-(4-methylpiperazin-1-yl)pyrimidin-4-yl]-N-[(1R)-1-(1-naphthyl)ethyl]benzamide (Example 137), using the required commercially available secondary amine.

| Example | Structure | Name and Analytical Data |
|---|---|---|
| 138 | 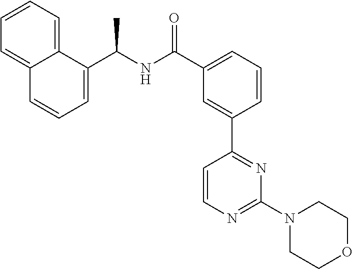 [SMILES: C[C@@H](NC(=O)c1cccc(-c2ccnc(N3CCOCC3)n2)c1)c1cccc2ccccc12] | 3-(2-Morpholinopyrimidin-4-yl)-N-[(1R)-1-(1- naphthyl)ethyl]benzamide ¹H NMR (500 MHz, CDCl₃) δ 8.43 (s, 1H), 8.41- 8.36 (m, 1H), 8.19 (d, J = 8.2, 1H), 8.17-8.13 (m, 1H), 7.91-7.87 (m, 1H), 7.84 (d, J = 7.9, 1H), 7.77 (d, J = 7.9, 1H), 7.62 (d, J = 7.0, 1H), 7.57-7.46 (m, 4H), 6.99 (d, J = 5.2, 1H), 6.40 (br d, J = 7.6, 1H), 6.15 (quin, J = 7.0, 1H), 3.87-3.83 (m, 4H), 3.79- 3.75 (m, 4H), 1.82 (d, J = 6.7, 3H). LC- MS (Method B): R_{T} = 3.96, m/z = 437.8 [M − H]⁻. |

### Example 139: 2-Methyl-N-[(1R)-1-(1-naphthyl)ethyl]-5-(4-pyridyl)benzamide

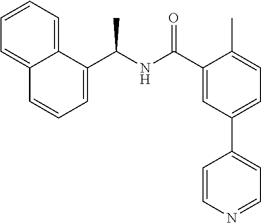 [SMILES: Cc1ccc(-c2ccncc2)cc1C(=O)N[C@H](C)c1cccc2ccccc12]

Using General Procedure 2 with 5-bromo-2-methyl-N-[(1R)-1-(1-naphthyl)ethyl]benzamide (217 mg, 589 μmol)—prepared in a similar manner to N-[(1R)-1-(1-naphthyl)ethyl]-3-(1-piperidyl)benzamide (Example 1)—and pyridine-4-boronic acid hydrate (91 mg, 64 μmol) at 85° C. for 3.5 hours gave 2-methyl-N-[(1R)-1-(1-naphthyl)ethyl]-5-(4-pyridyl)benzamide (115 mg, 48%) as a slightly off white crystalline solid. ¹H NMR (500 MHz, DMSO-d₆) δ 9.01 (d, J=8.0, 1H), 8.65 (d, J=6.0, 2H), 8.26 (d, J=8.0, 1H), 7.97 (m, 1H), 7.85 (d, J=8.0, 1H), 7.77 (dd, J=8.0, 2.0, 1H), 7.72 (m, 3H), 7.66 (d, J=7.0, 1H), 7.62 (ddd, J=8.5, 7.0, 1.5, 1H), 7.57-7.52 (m, 2H), 7.40 (d, J=8.0, 1H), 5.95 (quin, J=7.0, 1H), 2.36 (s, 3H), 1.63 (d, J=7.0, 3H). LC-MS (Method B): R_{T}=3.66, m/z=365.6 [M−H]⁻.

### Further Examples

The following examples were prepared in a similar manner to 2-methyl-N-[(1R)-1-(1-naphthyl)ethyl]-5-(4-pyridyl)benzamide (Example 139), using the required commercially available boronic ester or acid.

| Example | Structure | Name and Analytical Data |
|---|---|---|
| 140 | 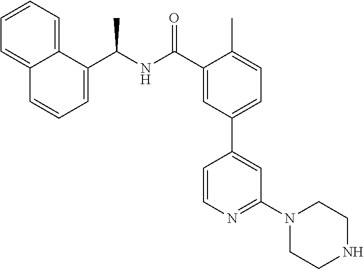 [SMILES: Cc1ccc(-c2ccnc(N3CCNCC3)c2)cc1C(=O)N[C@H](C)c1cccc2ccccc12] | 2-Methyl-N-[(1R)-1-(1-naphthyl)ethyl]-5-(2- piperazin-1-yl-4-pyridyl)benzamide ¹H NMR (500 MHz, DMSO-d₆) δ 8.99 (d, J = 8.0, 1H), 8.26 (d, J = 8.5, 1H), 8.16 (d, J = 5.0, 1H), 7.97 (m, 1H), 7.85 (d, J = 8.0, 1H), 7.71 (dd, J = 8.0, 2.0, 1H), 7.66-7.64 (m, 2H), 7.62 (ddd, J = 8.5, 7.0, 1.5, 1H), 7.57-7.51 (m, 2H), 7.34 (d, J = 8.0, 1H), 6.98 (br s, 1H), 6.93 (dd, J = 5.0, 1.5, 1H), 5.94 (quin, J = 7.0, 1H), 3.47 (m, 4H), 2.80 (m, 4H), 2.34 (s, 3H), 1.62 (d, J = 7.0, 3H). LC-MS (Method B): R_{T} = 3.83, m/z = 449.8 [M − H]⁻. |
| 141 | 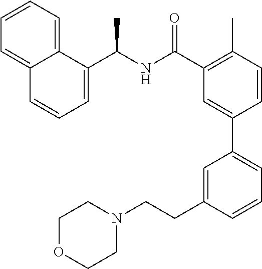 [SMILES: Cc1ccc(-c2cccc(CCN3CCOCC3)c2)cc1C(=O)N[C@H](C)c1cccc2ccccc12] | 2-Methyl-5-[3-(2-morpholinoethyl)phenyl]- N-[(1R)-1-(1-naphthyl)ethyl]benzamide ¹H NMR (500 MHz, CDCl₃) δ 8.27 (d, J = 8.5, 1H), 7.88 (d, J = 8.2, 1H), 7.82 (d, J = 7.9, 1H), 7.61-7.56 (m, 2H), 7.55-7.50 (m, 1H), 7.50- 7.44 (m, 3H), 7.33 (d, J = 2.4, 3H), 7.24-7.23 (m, 1H), 7.21-7.11 (m, 1H), 6.20-6.13 (m, 1H), 6.00 (br d, J = 8.2, 1H), 3.78-3.70 (m, 6H), 2.87- 2.80 (m, 2H), 2.63-2.55 (m, 2H), 2.53-2.50 (m, 2H), 2.46 (s, 3H), 1.82 (d, J = 6.7, 3H). LC- MS (Method B): R_{T} = 4.03, m/z = 477.9 [M − H]⁻. |
| 142 | 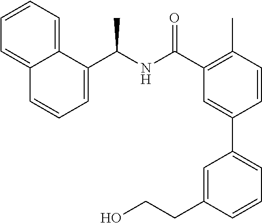 [SMILES: Cc1ccc(-c2cccc(CCO)c2)cc1C(=O)N[C@H](C)c1cccc2ccccc12] | 5-[3-(2-Hydroxyethyl)phenyl]-2-methyl-N- [(1R)-1-(1-naphthyl)ethyl]benzamide. ¹H NMR (500 MHz, CDCl₃) δ 8.27 (d, J = 8.5, 1H), 7.88 (d, J = 8.2, 1H), 7.82 (d, J = 8.2, 1H), 7.62-7.56 (m, 2H), 7.56-7.45 (m, 4H), 7.38- 7.33 (m, 3H), 7.26-7.24 (m, 1H), 7.23-7.17 (m, 1H), 6.19-6.13 (m, 1H), 6.00 (br d, J = 8.2, 1H), 3.88 (q, J = 6.4, 2H), 2.91 (t, J = 6.6, 2H), 2.46 (s, 3H), 1.82 (d, J = 6.7, 3H), 1.36 (t, J = 6.4, 1H). LC-MS (Method B): R_{T} = 3.83, m/z = 408.7 [M − H]⁻. |
| 143 | 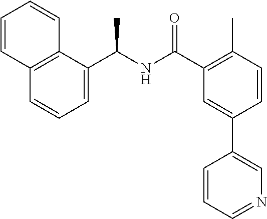 [SMILES: Cc1ccc(-c2cccnc2)cc1C(=O)N[C@H](C)c1cccc2ccccc12] | 2-Methyl-N-[(1R)-1-(1-naphthyl)ethyl]-5-(3- pyridyl)benzamide ¹H NMR (500 MHz, DMSO-d₆) δ 8.98 (d, J = 8.0, 1H), 8.92 (d, J = 2.0, 1H), 8.58 (dd, J = 5.0, 1.5, 1H), 8.26 (d, J = 8.0, 1H), 8.09 (br d, J = 8.0, 1H), 7.97 (d, J = 7.5, 1H), 7.85 (d, J = 8.0, 1H), 7.70-7.60 (m, 4H), 7.57-7.49 (m, 3H), 7.37 (d, J = 8.0, 1H), 5.95 (quin, J = 7.0, 1H), 2.36 (s, 3H), 1.62 (d, J = 7.0, 3H). LC-MS (Method B): R_{T} = 3.65, m/z = 365.6 [M − H]⁻. |

### Example 144: 2-Chloro-5-(1-methyl-3,6-dihydro-2H-pyridin-4-yl)-N-[(1R)-1-(1-naphthyl)ethyl]benzamide

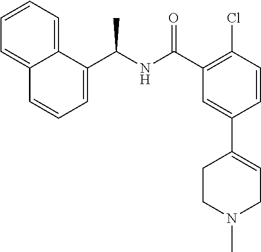 [SMILES: C[C@@H](NC(=O)c1cc(C2=CCN(C)CC2)ccc1Cl)c1cccc2ccccc12]

Using General Procedure 2 with 1-methyl-4-(4,4,5,5-tetramethyl-1,3,2-dioxaborolan-2-yl)-3,6-dihydro-2H-pyridine (123 mg, 551 μmol) and 2-chloro-5-iodo-N-[(1R)-1-(1-naphthyl)ethyl]benzamide (200 mg, 459 μmol)—prepared in a similar manner to N-[(1R)-1-(1-naphthyl)ethyl]-3-(1-piperidyl)benzamide (Example 1)—at 60° C. for 2 hours gave 2-chloro-5-(1-methyl-3,6-dihydro-2H-pyridin-4-yl)-N-[(1R)-1-(1-naphthyl)ethyl]benzamide (110 mg, 53%) as a white solid. ¹H NMR (500 MHz, CDCl₃) δ 8.25-8.21 (m, 1H), 7.88 (d, J=7.9, 1H), 7.85-7.76 (m, 1H), 7.63 (d, J=2.1, 1H), 7.61-7.45 (m, 4H), 7.33-7.27 (m, 2H), 6.49 (br d, J=7.9, 1H), 6.17-6.03 (m, 2H), 3.13-3.03 (m, 2H), 2.67-2.56 (m, 2H), 2.51-2.46 (m, 2H), 2.41-2.36 (m, 3H), 1.81 (d, J=6.7, 3H). LC-MS (Method B): R_{T}=3.88, m/z=403.6 [M−H]⁻.

### Further Examples

The following examples were prepared in a similar manner to 2-methyl-N-[(1R)-1-(1-naphthyl)ethyl]-5-(4-pyridyl)benzamide (Example 144), using the required commercially available boronic ester or acid.

| Example | | |
|---|---|---|
| (Intermediate) | Structure | Name and Analytical Data |
| 145 | 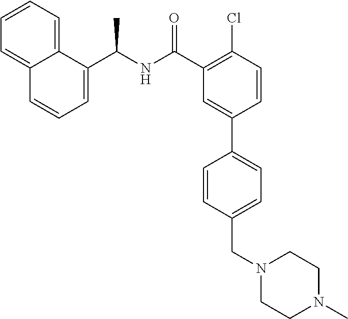 [SMILES: C[C@@H](NC(=O)c1cc(-c2ccc(CN3CCN(C)CC3)cc2)ccc1Cl)c1cccc2ccccc12] | 2-Chloro-5-[4-[(4-methylpiperazin-1- yl)methyl]phenyl]-N-[(1R)-1-(1- naphthyl)ethyl]benzamide ¹H NMR (500 MHz, CDCl₃) δ 8.25 (d, J = 8.5, 1H), 7.88 (d, J = 7.9, 1H), 7.86-7.80 (m, 2H), 7.62-7.57 (m, 2H), 7.54-7.45 (m, 5H), 7.45- 7.33 (m, 3H), 6.45 (br d, J = 7.9, 1H), 6.17 (quin, J = 7.2, 1H), 3.54 (s, 2H), 2.67-2.37 (m, 8H), 2.30 (s, 3H), 1.83 (d, J = 6.7, 3H). LC-MS (Method B): R_{T} = 4.09, m/z = 496.6 [M − H]⁻. |

### Example 146: 2-Chloro-5-(2,5-dihydro-1H-pyrrol-3-yl)-N-[(1R)-1-(1-naphthyl)ethyl]benzamide Hydrochloride Salt

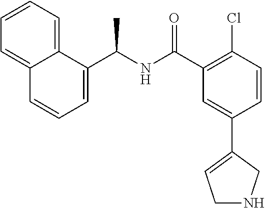 [SMILES: C[C@@H](NC(=O)c1cc(C2=CCNC2)ccc1Cl)c1cccc2ccccc12]

tert-Butyl 3-[4-chloro-3-[[(1R)-1-(1-naphthyl)ethyl]carbamoyl]phenyl]-2,5-dihydropyrrole-1-carboxylate (740 mg, 1.55 mmol)—prepared in a similar manner to 2-chloro-5-(1-methyl-3,6-dihydro-2H-pyridin-4-yl)-N-[(1R)-1-(1-naphthyl)ethyl]benzamide (Example 144)—was added to 6N HCl in propan-2-ol (20 mL) and stirred for 1 hour. The mixture was slowly evaporated until a solid was observed, diethyl ether was added, and the mixture stirred for 10 mins before being filtered. The obtained solid was dissolved in DCM and stirred for 20 mins before being filtered under nitrogen to afford 2-chloro-5-(2,5-dihydro-1H-pyrrol-3-yl)-N-[(1R)-1-(1-naphthyl)ethyl]benzamide hydrochloride salt (380 mg, 60%) as a white solid. ¹H NMR (500 MHz, DMSO-d₆) δ 9.80 (br s, 2H), 9.16 (d, J=7.9, 1H), 8.23 (d, J=8.5, 1H), 7.97 (d, J=7.9, 1H), 7.86 (d, J=8.2, 1H), 7.67 (d, J=7.0, 1H), 7.63-7.59 (m, 2H), 7.58-7.51 (m, 4H), 6.55 (t, J=2.0, 1H), 5.91 (quin, J=7.2, 1H), 4.34 (br s, 2H), 4.14 (br s, 2H), 1.60 (d, J=6.7, 3H). LC-MS (Method B): R_{T}=3.79, m/z=375.7 [M−H]⁻.

### Example 147: 2-chloro-N-[(1R)-1-(1-naphthyl)ethyl]-5-pyrrolidin-3-yl-benzamide

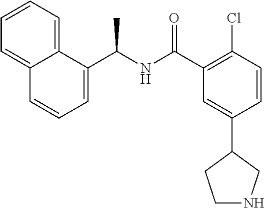 [SMILES: C[C@@H](NC(=O)c1cc(C2CCNC2)ccc1Cl)c1cccc2ccccc12]

Using General Procedure 5 with 2-chloro-5-(2,5-dihydro-1H-pyrrol-3-yl)-N-[(1R)-1-(1-naphthyl)ethyl]benzamide hydrochloride salt (360 mg, 955 μmol) (Example 146) and palladium, 10% on activated carbon powder (102 mg, 955 μmol) gave 2-chloro-N-[(1R)-1-(1-naphthyl)ethyl]-5-pyrrolidin-3-yl-benzamide (288 mg, 80%) as a white foam, after purification by SCX (eluted with 1M NH₃ in MeOH). ¹H NMR (500 MHz, CDCl₃) δ 8.23 (d, J=8.5, 1H), 7.88 (d, J=7.9, 1H), 7.81 (d, J=7.9, 1H), 7.62-7.55 (m, 2H), 7.54-7.45 (m, 3H), 7.30-7.25 (m, 2H), 7.19 (dd, J=2.1, 8.2, 1H), 6.50 (br d, J=7.9, 1H), 6.14 (quin, J=7.1, 1H), 3.30 (dd, J=7.8, 10.8, 1H), 3.20-3.02 (m, 3H), 2.75 (ddd, J=5.0, 8.0, 10.8, 1H), 2.24-2.16 (m, 1H), 1.87-1.72 (m, 4H). LC-MS (Method B): R_{T}=4.60, m/z=377.7 [M−H]⁻.

### Example 148: 1-Methyl-N-[(1R)-1-(1-naphthyl)ethyl]-4-(4-pyridyl)pyrrole-2-carboxamide

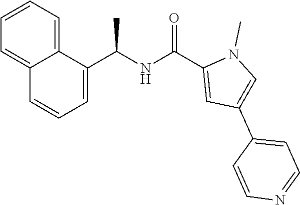 [SMILES: C[C@@H](NC(=O)c1cc(-c2ccncc2)cn1C)c1cccc2ccccc12]

Using General Procedure 2 with 4-bromo-1-methyl-N-[(1R)-1-(1-naphthyl)ethyl]pyrrole-2-carboxamide (200 mg, 560 μmol)—prepared in a similar manner to N-[(1R)-1-(1-naphthyl)ethyl]-3-(1-piperidyl)benzamide (Example 1)—and pyridine-4-boronic acid hydrate (79 mg, 560 μmol) at 80° C. for 1 hour gave 1-methyl-N-[(1R)-1-(1-naphthyl)ethyl]-4-(4-pyridyl)pyrrole-2-carboxamide (27 mg, 14%) as a white solid. ¹H NMR (500 MHz, CDCl₃) δ 8.49 (br s, 2H), 8.16 (d, J=8.2, 1H), 7.89 (d, J=8.2, 1H), 7.83 (d, J=8.2, 1H), 7.60 (d, J=7.3, 1H), 7.58-7.46 (m, 3H), 7.31-7.27 (m, 2H), 7.14 (d, J=1.8, 1H), 6.77 (d, J=1.5, 1H), 6.18 (br d, J=7.6, 1H), 6.06 (quin, J=7.0, 1H), 4.02 (s, 3H), 1.81-1.74 (m, 3H). LC-MS (Method B): R_{T}=3.66, m/z=354.6 [M−H]⁻.

### Example 149: 4-(2,5-Dihydro-1H-pyrrol-3-yl)-1-methyl-N-[(1R)-1-(1-naphthyl)ethyl]pyrrole-2-carboxamide Hydrochloride Salt

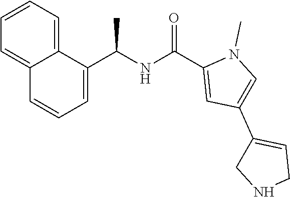 [SMILES: C[C@@H](NC(=O)c1cc(C2=CCNC2)cn1C)c1cccc2ccccc12]

tert-butyl 3-[1-methyl-5-[[(1R)-1-(1-naphthyl)ethyl]carbamoyl]pyrrol-3-yl]-2,5-dihydropyrrole-1-carboxylate (487 mg, 1.09 mmol)—prepared in a similar manner to 1-methyl-N-[(1R)-1-(1-naphthyl)ethyl]-4-(4-pyridyl)pyrrole-2-carboxamide (Example 148)—was added to 6N HCl in propan-2-ol (20 mL) and stirred for 1 hour. The mixture was evaporated until only 5 mL solvent remained, this was diluted with diethyl ether (15 mL) and stirred for 30 mins to afford a solid which was filtered under nitrogen to give 4-(2,5-dihydro-1H-pyrrol-3-yl)-1-methyl-N-[(1R)-1-(1-naphthyl)ethyl]pyrrole-2-carboxamide hydrochloride salt (388 mg, 84%) as a white solid. ¹H NMR (500 MHz, DMSO-d₆) δ 9.61 (br s, 1H), 8.58 (d, J=7.9, 1H), 8.19 (br d, J=8.2, 1H), 7.95 (br d, J=7.3, 1H), 7.85-7.81 (m, 1H), 7.64-7.46 (m, 5H), 7.19-7.15 (m, 1H), 5.89 (td, J=7.1, 14.5, 1H), 5.82-5.79 (m, 1H), 4.18-4.08 (m, 2H), 4.05 (br s, 1H), 3.84-3.72 (m, 4H), 1.62-1.55 (m, 3H).

### Example 150: 1-Methyl-N-[(1R)-1-(1-naphthyl)ethyl]-4-pyrrolidin-3-yl-pyrrole-2-carboxamide

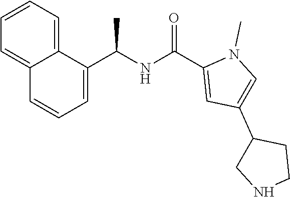 [SMILES: C[C@@H](NC(=O)c1cc(C2CCNC2)cn1C)c1cccc2ccccc12]

Using General Procedure 5 with 4-(2,5-dihydro-1H-pyrrol-3-yl)-1-methyl-N-[(1R)-1-(1-naphthyl)ethyl]pyrrole-2-carboxamide (287 mg, 830 μmol) (Example 149) and palladium, 10% on activated carbon powder (88 mg, 830 μmol) gave 1-methyl-N-[(1R)-1-(1-naphthyl)ethyl]-4-pyrrolidin-3-yl-pyrrole-2-carboxamide (23 mg, 8%) as a white foam. ¹H NMR (500 MHz, CDCl₃) δ 8.16 (d, J=8.2, 1H), 7.87 (d, J=7.6, 1H), 7.84-7.77 (m, 1H), 7.59-7.44 (m, 4H), 6.54 (d, J=1.5, 1H), 6.33 (s, 1H), 6.11 (br d, J=6.7, 1H), 6.06-5.99 (m, 1H), 3.96-3.86 (m, 3H), 3.22 (ddd, J=3.1, 7.2, 10.5, 1H), 3.12-2.97 (m, 2H), 2.78-2.69 (m, 1H), 2.18-2.00 (m, 3H), 1.76-1.65 (m, 4H). LC-MS (Method B): R_{T}=4.50, m/z=346.7[M−H]⁻.

### Example 151: 1-Methyl-N-[(1R)-1-(1-naphthyl)ethyl]-4-[4-(4-piperidyl)phenyl]pyrrole-2-carboxamide

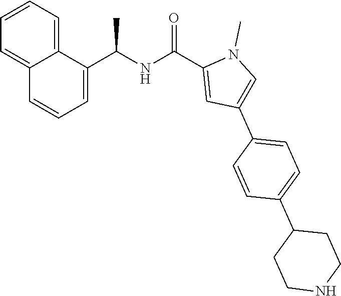 [SMILES: C[C@@H](NC(=O)c1cc(-c2ccc(C3CCNCC3)cc2)cn1C)c1cccc2ccccc12]

Using General Procedure 5 with benzyl 4-[4-[1-methyl-5-[[(1R)-1-(1-naphthyl)ethyl]carbamoyl]pyrrol-3-yl]phenyl]piperidine-1-carboxylate (130 mg, 227.39 μmol)—prepared in a similar manner to 1-methyl-N-[(1R)-1-(1-naphthyl)ethyl]-4-(4-pyridyl)pyrrole-2-carboxamide (Example 148)—and palladium, 10% on activated carbon powder (12 mg, 114 μmol) gave 1-methyl-N-[(1R)-1-(1-naphthyl)ethyl]-4-[4-(4-piperidyl)phenyl]pyrrole-2-carboxamide (43 mg, 42%) as a white solid. ¹H NMR (500 MHz, CDCl₃) δ 8.20-8.14 (m, 1H), 7.88 (d, J=7.6, 1H), 7.82 (d, J=8.2, 1H), 7.60 (d, J=7.0, 1H), 7.57-7.45 (m, 3H), 7.35 (dd, J=2.1, 8.2, 2H), 7.21-7.12 (m, 2H), 6.97 (d, J=1.8, 1H), 6.74-6.64 (m, 1H), 6.20 (br d, J=7.9, 1H), 6.09-5.99 (m, 1H), 4.00 (s, 3H), 3.34 (s, 1H), 3.23-3.10 (m, 1H), 3.07-2.94 (m, 1H), 2.80-2.69 (m, 1H), 2.68-2.53 (m, 1H), 2.52-2.40 (m, 1H), 2.14-2.02 (m, 1H), 1.90-1.56 (m, 6H). LC-MS (Method B): R_{T}=5.59 m/z=436.8 [M−H]⁻.

### Example 152: 2-Methyl-N-[(1R)-1-(1-naphthyl)ethyl]-5-(3-piperazin-1-ylsulfonylphenyl)benzamide

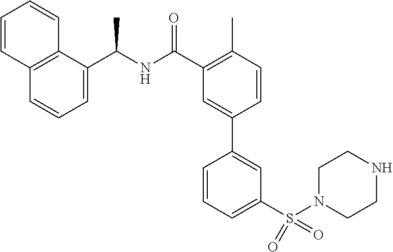 [SMILES: Cc1ccc(-c2cccc(S(=O)(=O)N3CCNCC3)c2)cc1C(=O)N[C@H](C)c1cccc2ccccc12]

Using General Procedure 4 with tert-butyl 4-[3-[4-methyl-3-[[(1R)-1-(1-naphthyl)ethyl]carbamoyl]phenyl]phenyl]sulfonylpiperazine-1-carboxylate (88 mg, 143 μmol)—prepared in a similar manner to 2-methyl-N-[(1R)-1-(1-naphthyl)ethyl]-5-(4-pyridyl)benzamide (Example 139)—gave 2-methyl-N-[(1R)-1-(1-naphthyl)ethyl]-5-(3-piperazin-1-ylsulfonylphenyl)benzamide (24 mg, 29%) as a tan crystalline solid.

¹H NMR (500 MHz, DMSO-d₆) δ 9.04 (d, J=8.0, 1H), 8.27 (d, J=8.5, 1H), 8.04 (dt, J=8.0, 1.5, 1H), 7.97 (d, J=7.5, 1H), 7.90 (t, J=1.5, 1H), 7.86 (d, J=8.0, 1H), 7.77 (m, 1H), 7.72 (m, 1H), 7.69 (dd, J=8.0, 2.0, 1H), 7.66 (d, J=7.0, 1H), 7.63-7.60 (m, 2H), 7.57-7.51 (m, 2H), 7.39 (d, J=8.0, 1H), 5.95 (quin, J=7.0, 1H), 2.83 (m, 4H), 2.73 (m, 4H), 2.37 (s, 3H), 1.62 (d, J=7.0, 3H). LC-MS (Method B): R_{T}=3.71, m/z=512.9 [M−H]⁻.

### Example 153: 2-Methyl-N-[(1R)-1-(1-naphthyl)ethyl]-5-(1H-pyrazol-4-yl)benzamide

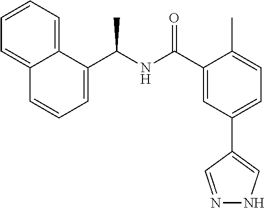 [SMILES: Cc1ccc(-c2cn[nH]c2)cc1C(=O)N[C@H](C)c1cccc2ccccc12]

### Step A: Benzyl 2-methyl-5-(1H-pyrazol-4-yl)benzoate

Using General Procedure 2 with benzyl 5-bromo-2-methyl-benzoate (2.20 g, 7.21 mmol) and 4-(4,4,5,5-tetramethyl-1,3,2-dioxaborolan-2-yl)-1H-pyrazole (1.40 g, 7.21 mmol) at 90° C. for 3 hours gave benzyl 2-methyl-5-(1H-pyrazol-4-yl)benzoate (320 mg, 15%) as a yellow gum. LC-MS (Method B): R_{T}=3.74, m/z=291.6 [M−H]⁻.

### Step B: 2-Methyl-5-(1H-pyrazol-4-yl)benzoic Acid

Using General Procedure 5 with benzyl 2-methyl-5-(1H-pyrazol-4-yl)benzoate (320 mg, 1.09 mmol) and palladium, 10% on activated carbon powder (58 mg, 547 μmol) gave 2-methyl-5-(1H-pyrazol-4-yl)benzoic acid (68 mg, 31%) as a white solid. LC-MS (Method B): R_{T}=0.30, m/z=Not observed.

### Step C: 2-Methyl-N-[(1R)-1-(1-naphthyl)ethyl]-5-(1H-pyrazol-4-yl)benzamide

Using General Procedure 1 with (1R)-1-(1-naphthyl)ethanamine (50 mg, 291.99 μmol) and 2-methyl-5-(1H-pyrazol-4-yl)benzoic acid (64.95 mg, 321.19 μmol) gave 2-methyl-N-[(1R)-1-(1-naphthyl)ethyl]-5-(1H-pyrazol-4-yl)benzamide (35 mg, 34%) as a white solid. ¹H NMR (500 MHz, DMSO-d₆) δ 12.94 (br s, 1H), 8.92 (d, J=7.9, 1H), 8.26 (d, J=8.2, 1H), 3.21 (brs, 1H), 7.97 (d, J=8.2, 1H), 7.90 (brs, 1H), 7.86 (s, 1H), 7.68-7.59 (m, 2H), 7.58-7.50 (m, 4H), 7.20 (d, J=7.9, 1H), 5.93 (quin, J=7.1, 1H), 2.26 (s, 3H), 1.61 (d, J=7.0, 3H). LC-MS (Method B): R_{T}=3.38, m/z=354.7 [M−H]⁻.

### Example 154: 2-Methyl-N-[(1R)-1-(1-naphthyl)ethyl]-5-(1,2,3,6-tetrahydropyridin-4-yl)benzamide

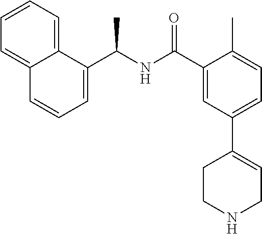 [SMILES: Cc1ccc(C2=CCNCC2)cc1C(=O)N[C@H](C)c1cccc2ccccc12]

### Step A: 2-Methyl-N-[(1R)-1-(1-naphthyl)ethyl]-5-(4,4,5,5-tetramethyl-1,3,2-dioxaborolan-2-yl)benzamide

5-Bromo-2-methyl-N-[(1R)-1-(1-naphthyl)ethyl]benzamide (1.00 g, 2.72 mmol)—prepared in a similar manner to N-[(1R)-1-(1-Naphthyl)ethyl]-3-(1-piperidyl)benzamide (Example 1)—4,4,5,5-tetramethyl-2-(4,4,5,5-tetramethyl-1,3,2-dioxaborolan-2-yl)-1,3,2-dioxaborolane (759 mg, 2.99 mmol), 1,1′-bis(diphenylphosphino)ferrocenepalladium(II) dichloride (99 mg, 136 μmol) and potassium acetate (799 mg, 8.15 mmol) were added to DMSO (30 mL) and heated at 90° C. for 2 hours. The black mixture was cooled, diluted with water (100 mL), extracted with diethyl ether (2×75 mL), dried (MgSO₄) and solvent removed in vacuo to afford a black gum. Purification by FCC (eluting with 20-40% diethyl ether in petroleum ether) afforded 2-methyl-N-[(1R)-1-(1-naphthyl)ethyl]-5-(4,4,5,5-tetramethyl-1,3,2-dioxaborolan-2-yl)benzamide (580 mg, 51%) as a white foam. LC-MS (Method B): R_{T}=4.22, m/z=416.6 [M−H]⁺.

### Step B: tert-Butyl 4-[4-methyl-3-[[(1R)-1-(1-naphthyl)ethyl]carbamoyl]phenyl]-3,6-dihydro-2H-pyridine-1-carboxylate

Using General procedure 2 with 2-methyl-N-[(1R)-1-(1-naphthyl)ethyl]-5-(4,4,5,5-tetramethyl-1,3,2-dioxaborolan-2-yl)benzamide (250 mg, 602 μmol) and N-Boc-4-trifluoromethanesulfonyloxy-3,6-dihydro-2H-pyridine (219 mg, 662 μmol) at 80° C. for 1 hour gave tert-butyl 4-[4-methyl-3-[[(1R)-1-(1-naphthyl)ethyl]carbamoyl]phenyl]-3,6-dihydro-2H-pyridine-1-carboxylate (140 mg, 49%) as a brown foam. LC-MS (Method B): R_{T}=4.42, m/z=469.9 [M−H]⁻.

### Step C: 2-Methyl-N-[(1R)-1-(1-naphthyl)ethyl]-5-(1,2,3,6-tetrahydropyridin-4-yl)benzamide Hydrochloride Salt

6N HCl in propan-2-ol (297 μmol, 10 mL) was added to tert-butyl 4-[4-methyl-3-[[(1R)-1-(1-naphthyl)ethyl]carbamoyl]phenyl]-3,6-dihydro-2H-pyridine-1-carboxylate (140 mg, 297 μmol) and stirred for 1 hour to afford a yellow solution. The mixture was evaporated until only 50% volume was left, the mixture was triturated with diethyl ether and stirred for 10 mins before being filtered under nitrogen to afford 2-methyl-N-[(1R)-1-(1-naphthyl)ethyl]-5-(1,2,3,6-tetrahydropyridin-4-yl)benzamide hydrochloride salt (101 mg, 83%) as a beige solid. ¹H NMR (500 MHz, DMSO-d₆) δ 9.18 (br s, 2H), 8.96 (d, J=7.9, 1H), 8.25 (d, J=8.5, 1H), 7.97 (d, J=7.9, 1H), 7.85 (d, J=8.2, 1H), 7.64-7.58 (m, 2H), 7.57-7.50 (m, 2H), 7.45-7.41 (m, 1H), 7.38 (d, J=1.8, 1H), 7.25 (d, J=7.9, 1H), 6.20 (br s, 1H), 5.93 (quin, J=7.1, 1H), 3.74 (br s, 2H), 3.34-3.27 (m, 2H), 2.70-2.64 (m, 2H), 2.29 (s, 3H), 1.60 (d, J=7.0, 3H). LC-MS (Method B): R_{T}=4.36, m/z=369.7 [M−H]⁻.

### Further Examples

The following examples were prepared in a similar manner to 2-methyl-N-[(1R)-1-(1-naphthyl)ethyl]-5-(1,2,3,6-tetrahydropyridin-4-yl)benzamide (Example 154), using the appropriate aromatic bromide—prepared in a similar manner to N-[(1R)-1-(1-naphthyl)ethyl]-3-(1-piperidyl)benzamide (Example 1)—in Step A.

| Example | Structure | Name and Analytical Data |
|---|---|---|
| 155 | 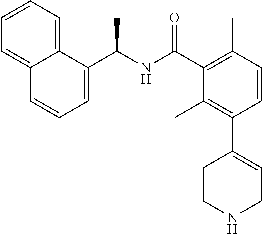 [SMILES: Cc1ccc(C2=CCNCC2)c(C)c1C(=O)N[C@H](C)c1cccc2ccccc12] | 2,6-Dimethyl-N-[(1R)-1-(1-naphthyl)ethyl]-3- (1,2,3,6-tetrahydropyridin-4-yl)benzamide ¹H NMR (500 MHz, CDCl₃) δ 8.29 (d, J = 8.5, 1H), 7.87 (d, J = 7.9, 1H), 7.81 (d, J = 7.9, 1H), 7.60-7.51 (m, 3H), 7.47-7.43 (m, 1H), 6.94 (s, 1H), 6.22-6.16 (m, 1H), 5.88 (d, J = 8.5, 1H), 5.49 (br s, 1H), 3.76 (br s, 2H), 3.35 (br s, 2H), 2.55 (br s, 2H), 2.28 (s, 3H), 2.22 (s, 3H), 1.81 (d, J = 6.7, 3H). LC-MS (Method B): R_{T} = 4.27, m/z = [M − H]⁻. |

### Example 156: 2-Methyl-N-[(1R)-1-(1-napthyl)ethyl]-5-(4-piperidyl)benzamide

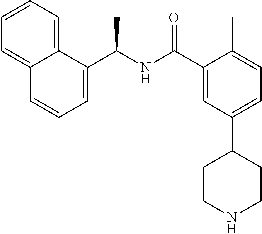 [SMILES: Cc1ccc(C2CCNCC2)cc1C(=O)N[C@H](C)c1cccc2ccccc12]

Using General Procedure 5 with 2-methyl-N-[(1R)-1-(1-naphthyl)ethyl]-5-(1,2,3,6-tetrahydropyridin-4-yl)benzamide hydrochloride salt (91 mg, 224 μmol) (Example 154) and palladium, 10% on activated carbon powder (12 mg, 112 μmol) gave 2-methyl-N-[(1R)-1-(1-naphthyl)ethyl]-5-(4-piperidyl)benzamide (69 mg, 83%) as a white foam. ¹H NMR (500 MHz, CDCl₃) δ 8.25 (d, J=8.5, 1H), 7.88 (d, J=7.9, 1H), 7.84-7.80 (m, 1H), 7.60-7.56 (m, 2H), 7.56-7.45 (m, 2H), 7.16-7.08 (m, 3H), 6.17-6.11 (m, 1H), 5.94 (br d, J=8.2, 1H), 3.15 (m, 2H), 2.69 (dt, J=2.3, 12.1, 2H), 2.53 (m, 1H), 2.39 (s, 3H), 1.80 (d, J=6.7, 3H), 1.77-1.71 (m, 2H), 1.61-1.50 (m, 3H). LC-MS (Method B): R_{T}=5.68, m/z=371.7 [M−H]⁻.

### Further Examples

The following examples were prepared in a similar manner to 2-methyl-N-[(1R)-1-(1-naphthyl)ethyl]-5-(4-piperidyl)benzamide (Example 156), using the appropriate intermediate prepared in a similar manner to 2-methyl-N-[(1R)-1-(1-naphthyl)ethyl]-5-(1,2,3,6-tetrahydropyridin-4-yl)benzamide (Example 136), without performing Step C.

| Example | Structure | Name and Analytical Data |
|---|---|---|
| 157 | 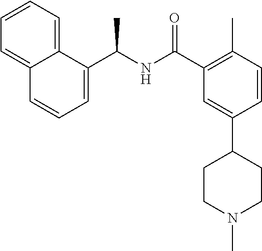 [SMILES: Cc1ccc(C2CCN(C)CC2)cc1C(=O)N[C@H](C)c1cccc2ccccc12] | 2-Methyl-5-(1-methyl-4-piperidyl)-N-[(1R)-1-(1- naphthyl)ethyl]benzamide ¹H NMR (500 MHz, DMSO-d₆) δ 8.84 (d, J = 7.9, 1H), 8.24 (d, J = 8.5, 1H), 7.96 (d, J = 7.9, 1H), 7.85 (d, J = 8.1, 1H), 7.68-7.48 (m, 4H), 7.22-7.09 (m, 3H), 5.92 (t, J = 7.3, 1H), 2.85 (br d, J = 11.3, 2H), 2.47-2.39 (m, 2H), 2.25 (s, 3H), 2.19 (s, 3H), 2.02- 1.88 (m, 2H), 1.76-1.62 (m, 2H), 1.59 (d, J = 6.9, 3H). LC-MS (Method B): R_{T} = 4.45, m/z = 385.8 [M − H]⁻. |

### Example 158: N-[(1R)-1-(1-Naphthyl)ethyl]-3-piperazin-1-yl-benzamide Hydrochloride Salt

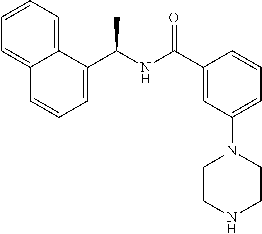 [SMILES: C[C@@H](NC(=O)c1cccc(N2CCNCC2)c1)c1cccc2ccccc12]

### Step A: tert-Butyl 4-[3-[[(1R)-1-(1-naphthyl)ethyl]carbamoyl]phenyl]piperazine-1-carboxylate

(1-Cyano-2-ethoxy-2-oxoethylidene aminooxy)dimethylaminomorpholino carbenium hexafluorophosphate (399 mg, 931 μmol) was added to a solution of (1R)-1-(1-naphthyl)ethanamine (145 mg, 847 μmol), 3-{4-[(tert-butoxy)carbonyl]piperazin-1-yl}benzoic acid (259 mg, 847 μmol) and DIPEA (434 μL, 2.54 mmol) in DMF (3 mL) and the reaction mixture allowed to stir for 0.5 hours. Water (70 mL) was added and the resulting pink solid was filtered and dried under vacuum which gave tert-butyl 4-[3-[[(1R)-1-(1-naphthyl)ethyl]carbamoyl]phenyl]piperazine-1-carboxylate (382 mg, 98%) as a pink solid. LC-MS (Method B): R_{T}=4.20, m/z=458.9 [M−H]⁻.

### Step B: N-[(1R)-1-(1-Naphthyl)ethyl]-3-piperazin-1-yl-benzamide Hydrochloride Salt

Using General Procedure 4 with tert-butyl 4-[3-[[(1R)-1-(1-naphthyl)ethyl]carbamoyl]phenyl]piperazine-1-carboxylate (383 mg, 833 μmol) gave N-[(1R)-1-(1-naphthyl)ethyl]-3-piperazin-1-yl-benzamide hydrochloride salt (85 mg, 23%) as a pale yellow solid. ¹H NMR (500 MHz, DMSO-d₆) δ 9.23 (br s, 2H), 9.01 (d, J=8.0, 1H), 8.27 (d, J=8.5, 1H), 8.02 (m, 1H), 7.91 (d, J=8.5, 1H), 7.72 (d, J=7.0, 1H), 7.66-7.56 (m, 3H), 7.54 (m, 1H), 7.49 (d, J=7.5, 1H), 7.41 (m, 1H), 7.22 (m, 1H), 6.03 (quin, J=7.0, 1H), 3.48 (m, 4H), 3.28 (m, 4H), 1.70 (d, J=7.0, 3H). LC-MS (Method B): R_{T}=3.41, m/z=358.7 [M−H]⁻.

### Example 159: 3-(4-Acetylpiperazin-1-yl)-N-[(1R)-1-(1-naphthyl)ethyl]benzamide

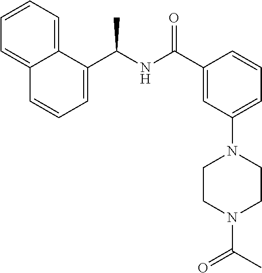 [SMILES: CC(=O)N1CCN(c2cccc(C(=O)N[C@H](C)c3cccc4ccccc34)c2)CC1]

Acetyl chloride (11.0 μL, 145 μmol) was added to a solution of N-[(1R)-1-(1-naphthyl)ethyl]-3-piperazin-1-yl-benzamide hydrochloride salt (Example 139) (62.0 mg, 172 μmol) and triethylamine (60.0 μL, 431 μmol) in DCM (5 mL) and the reaction mixture stirred at RT for 3 hours. The reaction mixture was diluted with 2 M aqueous HCl (10 mL) and DCM (5 mL) and the resulting layers separated. The aqueous layer was further extracted with DCM (2×10 mL) and the combined organics dried (MgSO₄), filtered, and concentrated in vacuo to afford an oil. The oil was slurried in DCM/petroleum ether and concentrated under reduced pressure to afford 3-(4-acetylpiperazin-1-yl)-N-[(1R)-1-(1-naphthyl)ethyl]benzamide as a white solid (42 mg, 58%). ¹H NMR (500 MHz, DMSO-d₆) δ 8.88 (d, J=7.6, 1H), 8.20 (d, J=8.7, 1H), 7.95 (d, J=8.2, 1H), 7.84 (d, J=8.1, 1H), 7.64 (d, J=7.2, 1H), 7.61-7.50 (m, 3H), 7.44 (s, 1H), 7.37-7.35 (m, 1H), 7.33-7.29 (m, 1H), 7.12 (br d, J=8.1, 1H), 5.96 (t, J=7.2, 1H), 3.61-3.55 (m, 4H), 3.21-3.19 (m, 2H), 3.15-3.13 (m, 2H) 2.04 (s, 3H), 1.62 (d, J=6.9, 3H). LC-MS (Method B): R_{T}=3.33, m/z=400.8 [M−H]⁻.

### Example 160: Methyl 3-[4-[3-[[(1R)-1-(1-naphthyl)ethyl]carbamoyl]phenyl]piperazin-1-yl]propanoate

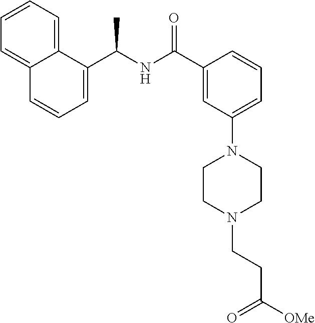 [SMILES: COC(=O)CCN1CCN(c2cccc(C(=O)N[C@H](C)c3cccc4ccccc34)c2)CC1]

Methyl acrylate (30 μL, 328 μmol) was added to a solution of N-[(1R)-1-(1-naphthyl)ethyl]-3-piperazin-1-yl-benzamide hydrochloride salt (Example 158) (59 mg, 164 μmol) and DIPEA (84 μL, 492 μmol) in MeOH (5 mL) at RT and the reaction mixture allowed to stir for 2 hours. The solvent was removed in vacuo and purification by FCC (eluting with 0-30% MeOH in DCM) gave methyl 3-[4-[3-[[(1R)-1-(1-naphthyl)ethyl]carbamoyl]phenyl]piperazin-1-yl]propanoate (44 mg, 57%) as a white crystalline solid. ¹H NMR (500 MHz, DMSO-d₆) δ 8.86 (d, J=7.5, 1H), 8.20 (d, J=8.0, 1H), 7.95 (m, 1H), 7.84 (d, J=8.0, 1H), 7.63 (d, J=7.0, 1H), 7.59-7.49 (m, 3H), 7.41 (m, 1H), 7.33 (m, 1H), 7.28 (m, 1H), 7.07 (m, 1H), 5.96 (quin, J=7.0, 1H), 3.59 (s, 3H), 3.15 (m, 4H), 2.62 (t, J=7.0, 2H), 1.62 (d, J=7.0, 3H). LC-MS (Method B): R_{T}=3.66, m/z=446.7 [M+H]⁺.

### Example 161: 3-[4-[3-[[(1R)-1-(1-Naphthyl)ethyl]carbamoyl]phenyl]piperazin-1-yl]propanoic Acid

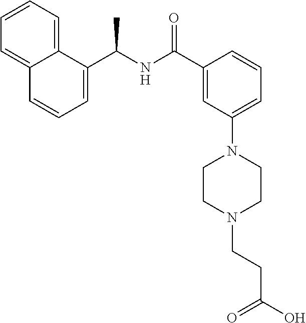 [SMILES: C[C@@H](NC(=O)c1cccc(N2CCN(CCC(=O)O)CC2)c1)c1cccc2ccccc12]

### Step A: Benzyl 3-[4-[3-[[(1R)-1-(1-naphthyl)ethyl]carbamoyl]phenyl]piperazin-1-yl]propanoate

DIPEA (141 μL, 826 μmol) was added to a solution of N-[(1R)-1-(1-naphthyl)ethyl]-3-piperazin-1-yl-benzamide hydrochloride salt (Example 158) (109 mg, 275 μmol) and benzyl acrylate (84.25 μL, 551 μmol) in MeOH (20 mL) at RT and the reaction mixture allowed to stir for 2 hours. The solvent was then removed in vacuo and purification by FCC eluting with 0-30% MeOH in DCM gave benzyl 3-[4-[3-[[(1R)-1-(1-naphthyl)ethyl]carbamoyl]phenyl]piperazin-1-yl]propanoate (103 mg, 72%) as a pale yellow oil. LC-MS (Method B): R_{T}=4.24, m/z=522.7 [M+H]⁺.

### Step B: 3-[4-[3-[[(1R)-1-(1-Naphthyl)ethyl]carbamoyl]phenyl]piperazin-1-yl]propanoic Acid

Using General Procedure 5 with palladium, 10% on activated carbon powder (30 mg) and benzyl 3-[4-[3-[[(1R)-1-(1-naphthyl)ethyl]carbamoyl]phenyl]piperazin-1-yl]propanoate (103 mg, 197 μmol) with purification by FCC (eluting with 0-100% MeOH in DCM) gave 3-[4-[3-[[(1R)-1-(1-naphthyl)ethyl]carbamoyl]phenyl]piperazin-1-yl]propanoic acid (52 mg, 55%) as an off-white solid. ¹H NMR (500 MHz, DMSO-d₆) δ 8.87 (d, J=8.0, 1H), 8.20 (d, J=8.5, 1H), 7.95 (m, 1H), 7.84 (d, J=8.5, 1H), 7.63 (d, J=7.0, 1H), 7.59-7.49 (m, 3H), 7.42 (m, 1H), 7.32 (m, 1H), 7.28 (m, 1H), 7.08 (dd, J=8.5, 1.5, 1H), 5.96 (quin, J=7.0, 1H), 3.17 (m, 4H), 2.61 (t, J=7.5, 2H), 2.55 (m, 4H), 2.40 (t, J=7.5, 2H), 1.62 (d, J=7.0, 3H). LC-MS (Method B): R_{T}=2.15, m/z=430.8 [M−H]⁻.

### Further Examples

The following examples were prepared in a similar manner to 3-[4-[3-[[(1R)-1-(1-naphthyl)ethyl]carbamoyl]phenyl]piperazin-1-yl]propanoic acid (Example 161), using in Step A the intermediate example stated.

| Example | | |
|---|---|---|
| (Intermediate) | Structure | Name and Analytical Data |
| 162 (Example 108) | 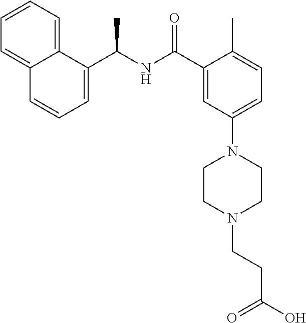 [SMILES: Cc1ccc(N2CCN(CCC(=O)O)CC2)cc1C(=O)N[C@H](C)c1cccc2ccccc12] | 3-[4-[4-Methyl-3-[[(1R)-1-(1- naphthyl)ethyl]carbamoyl]phenyl] piperazin-1-yl]propanoic acid ¹H NMR (500 MHz, DMSO-d₆) δ 8.81 (d, J = 8.0, 1H), 8.24 (d, J = 8.0, 1H), 7.96 (d, J = 7.5, 1H), 7.84 (d, J = 8.0, 1H), 7.63 (d, J = 7.0, 1H), 7.59 (m, 1H), 7.56-7.50 (m, 2H), 7.05 (d, J = 8.0, 1H), 6.89 (dd, J = 8.0, 3.0, 1H), 6.86 (d, J = 3.0, 1H), 5.89 (quin, J = 7.0, 1H), 3.08 (m, 4H), 2.60 (t, J = 7.0, 2H), 2.53 (m, 4H), 2.38 (t, J = 7.0, 2H), 2.17 (s, 3H), 1.58 (d, J = 7.0, 3H). LC-MS (Method B): R_{T} = 2.06, m/z = 444.8 [M − H]−. |
| 163 (Example 128) | 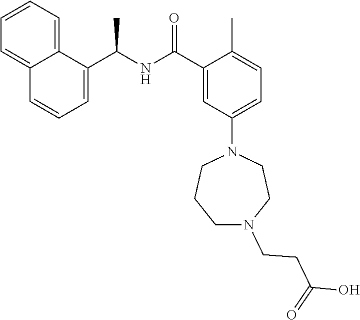 [SMILES: Cc1ccc(N2CCCN(CCC(=O)O)CC2)cc1C(=O)N[C@H](C)c1cccc2ccccc12] | 3-[4-[4-Methyl-3-[[(1R)-1-(1- naphthyl)ethyl]carbamoyl]phenyl]- 1,4-diazepan-1-yl]propanoic acid ¹H NMR (500 MHz, DMSO-d₆) δ 8.79 (d, J = 8.0, 1H), 8.24 (d, J = 8.0, 1H), 7.95 (m, 1H), 7.84 (d, J = 8.0, 1H), 7.62 (d, J = 7.0, 1H), 7.59 (m, 1H), 7.56-7.50 (m, 2H), 6.98 (d, J = 8.5, 1H), 6.65 (dd, J = 8.0, 3.0, 1H), 6.59 (d, J = 3.0, 1H), 5.69 (quin, J = 7.0, 1H), 3.45 (m, 2H), 3.41 (m, 4H), 2.75-2.70 (m, 4H), 2.56 (m, 2H), 2.31 (t, J = 7.0, 2H), 2.14 (s, 3H), 1.84 (m, 2H), 1.58 (d, J = 7.0, 3H). LC-MS (Method B): R_{T} = 2.30, m/z = 458.9 [M − H]⁻. |
| 164 (Example 6) | 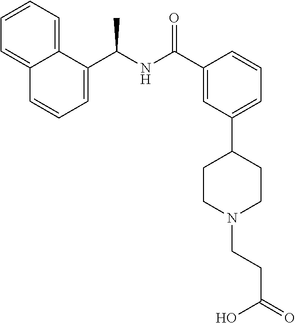 [SMILES: C[C@@H](NC(=O)c1cccc(C2CCN(CCC(=O)O)CC2)c1)c1cccc2ccccc12] | 3-[4-[3-[[(1R)-1-(1- Naphthyl)ethyl]carbamoyl]phenyl]- 1-piperidyl]propanoic acid ¹H NMR (500 MHz, DMSO-d₆) δ 8.93 (br d, J = 7.3, 1H), 8.21 (d, J = 8.2, 1H), 7.95 (d, J = 7.9, 1H), 7.87-7.79 (m, 2H), 7.74 (br d, J = 7.3, 1H), 7.65 (d, J = 6.7, 1H), 7.59-7.49 (m, 3H), 7.42- 7.36 (m, 2H), 5.97 (quin, J = 7.1, 1H), 3.03 (br d, J = 8.5, 2H), 2.64 (br s, 2H), 2.45-2.28 (m, 4H), 2.21-1.99 (m, 2H), 1.87-1.66 (m, 4H), 1.66-1.59 (m, 3H). LC-MS (Method B): R_{T} = 2.49, m/z = 429.8 [M − H]⁺. |
| 165 (Example 73) | 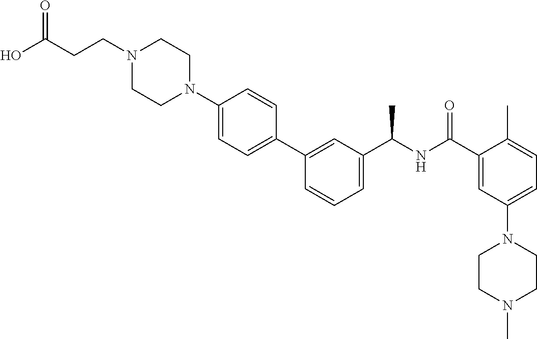 [SMILES: Cc1ccc(N2CCN(C)CC2)cc1C(=O)N[C@H](C)c1cccc(-c2ccc(N3CCN(CCC(=O)O)CC3)cc2)c1] | 3-[4-[4-[3-[(1R)-1-[[2-Methyl-5-(4- methylpiperazin-1- yl)benzoyl]amino]ethyl]phenyl] phenyl]piperazin-1-yl]propanoic acid ¹H NMR (500 MHz, DMSO-d₆) δ 8.69 (d, J = 8.0, 1H), 7.65 (br s, 1H), 7.53 (d, J = 8.5, 2H), 7.46 (d, J = 7.5, 1H), 7.37 (t, J = 7.5, 1H), 7.28 (d, J = 7.5, 1H), 7.06 (d, J = 8.5, 1H), 7.02 (d, J = 8.5, 2H), 6.91 (dd, J = 8.5, 2.5, 1H), 6.86 (d, J = 2.5, 1H), 5.16 (quin, J = 7.0, 1H), 3.19 (m, 4H), 3.09 (m, 4H), 2.62 (t, J = 7.0, 2H), 2.57 (m, 4H), 2.44-2.41 (m, 6H), 2.23 (s, 3H), 2.18 (s, 3H), 1.46 (d, J = 7.0, 3H). LC-MS (Method B): R_{T} = 2.19, m/z = 568.9 [M − H]⁻. |
| 166 (Example 105) | 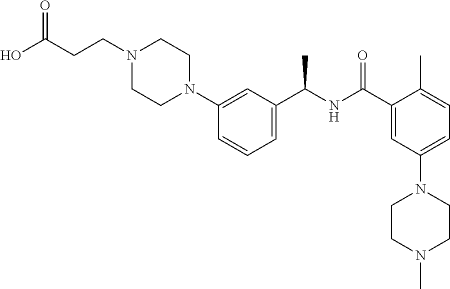 [SMILES: Cc1ccc(N2CCN(C)CC2)cc1C(=O)N[C@H](C)c1cccc(N2CCN(CCC(=O)O)CC2)c1] | 3-[4-[3-[(1R)-1-[[2-Methyl-5-(4- methylpiperazin-1- yl)benzoyl]amino]ethyl]phenyl]piper- azin-1-yl]propanoic acid ¹H NMR (500 MHz, DMSO-d₆) δ 8.58 (d, J = 8.0, 1H), 7.16 (t, J = 8.0, 1H), 7.06 (d, J = 8.5, 1H), 6.99 (m, 1H), 6.91 (dd, J = 8.5, 2.5, 1H), 6.83 (d, J = 3.0, 1H), 6.79 (dd, J = 8.0, 2.0, 2H), 5.05 (quin, J = 7.0, 1H), 3.13-3.09 (m, 8H), 2.61 (t, J = 7.0 Hz, 2H), 2.55 (m, 4H), 2.46 (m, 4H), 2.40 (t, J = 7.0, 2H), 2.23 (s, 3H), 2.18 (s, 3H), 1.39 (d, J = 7.5, 3H). LC-MS (Method B): R_{T} = 0.35, m/z = 492.9 [M − H]⁻. |
| 167 (Example 74) | 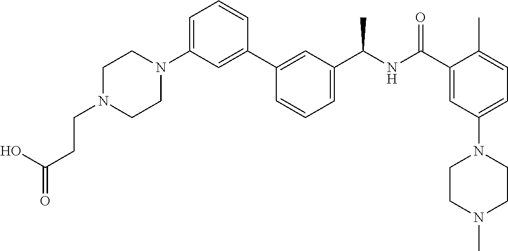 [SMILES: Cc1ccc(N2CCN(C)CC2)cc1C(=O)N[C@H](C)c1cccc(-c2cccc(N3CCN(CCC(=O)O)CC3)c2)c1] | 3-[4-[3-[3-[(1R)-1-[[2-Methyl-5-(4- methylpiperazin-1- yl)benzoyl]amino]ethyl]phenyl]phe- nyl]piperazin-1-yl]propanoic acid ¹H NMR (500 MHz, DMSO-d₆) δ 8.70 (d, J = 8.0, 1H), 7.68 (br s, 1H), 7.51 (d, J = 7.5, 1H), 7.40 (t, J = 7.5, 1H), 7.36 (br d, J = 7.5, 1H), 7.29 (t, J = 8.0, 1H), 7.14 (br s, 1H), 7.06 (m, 2H), 6.95 (dd, J = 8.0, 2.0, 1H), 6.91 (dd, J = 8.5, 2.5 Hz, 1H), 6.86 (d, J = 2.5, 1H), 5.18 (quin, J = 7.0, 1H), 3.20 (m, 4H), 3.09 (m, 4H), 2.63 (t, J = 7.0, 2H), 2.57 (m, 4H), 2.45-2.41 (m, 6H), 2.22 (s, 3H), 2.18 (s, 3H), 1.47 (d, J = 7.0, 3H). LC-MS (Method B): R_{T} = |
| | | 2.22, m/z = 569.0 [M − H]⁻. |
| 168 (Example 17) | 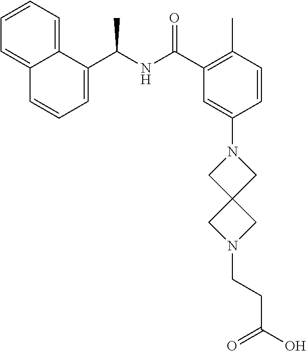 [SMILES: Cc1ccc(N2CC3(CN(CCC(=O)O)C3)C2)cc1C(=O)N[C@H](C)c1cccc2ccccc12] | 3-[2-[4-Methyl-3-[[(1R)-1-(1- naphthyl)ethyl]carbamoyl]phenyl]- 2,6-diazaspiro[3.3]heptan-6- yl]propanoic acid ¹H NMR (500 MHz, CDCl₃) δ 8.20 (d, J = 8.5, 1H), 7.85 (d, J = 7.9, 1H), 7.81- 7.74 (m, 1H), 7.58-7.52 (m, 1H), 7.52- 7.46 (m, 2H), 7.45-7.37 (m, 1H), 6.99- 6.92 (m, 1H), 6.36-6.31 (m, 1H), 6.31- 6.25 (m, 1H), 6.17-6.01 (m, 2H), 3.87- 3.70 (m, 4H), 3.70-3.51 (m, 4H), 2.94- 2.76 (m, 2H), 2.36-2.27 (m, 2H), 2.23 (s, 3H), 1.74 (d, 3H). LC-MS (Method A): R_{T} = 3.26, m/z = 458.5 [M + H]⁺. |
| 169 (Example 140) | 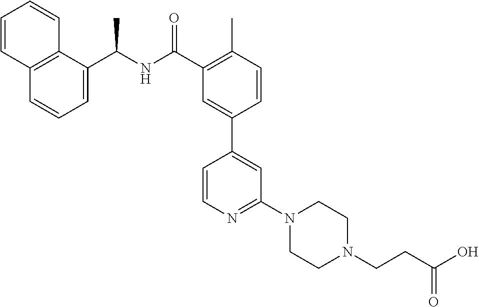 [SMILES: Cc1ccc(-c2ccnc(N3CCN(CCC(=O)O)CC3)c2)cc1C(=O)N[C@H](C)c1cccc2ccccc12] | 3-[4-[4-[4-Methyl-3-[[(1R)-1-(1- naphthyl)ethyl]carbamoyl]phenyl]- 2-pyridyl]piperazin-1-yl]propanoic acid ¹H NMR (500 MHz, DMSO-d₆) δ 8.99 (d, J = 8.0, 1H), 8.26 (d, J = 8.5, 1H), 8.17 (d, J = 5.0, 1H), 7.97 (d, J = 7.5, 1H), 7.85 (d, J = 8.0, 1H), 7.71 (dd, J = 8.0, 2.0, 1H), 7.65 (m, 2H), 7.62 (ddd, J = 8.5, 7.0, 1.0, 1H), 7.57- 7.51 (m, 2H), 7.35 (d, J = 8.0, 1H), 7.03 (s, 1H), 6.95 (dd, J = 5.0, 1.0, 1H), 5.94 (quin, J = 7.0, 1H), 3.54 (m, 4H), 2.62 (t, J = 7 .5, 2H), 2.42 (t, J = 7.5, 2H), 2.34 (s, 3H), 1.62 (d, J = 7.0, 3H). LC-MS (Method B): R_{T} = 2.39, m/z = 521.9 [M − H]⁻. |

### Example 170: 3-[4-[3-[[(1R)-1-[3-[3-(Hydroxymethyl)phenyl]phenyl]ethyl]carbamoyl]-4-methyl-phenyl]piperazin-1-yl]propanoic Acid

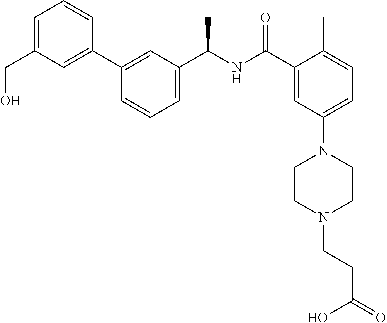 [SMILES: Cc1ccc(N2CCN(CCC(=O)O)CC2)cc1C(=O)N[C@H](C)c1cccc(-c2cccc(CO)c2)c1]

### Step A: tert-Butyl 4-[3-[[(1R)-1-[3-[3-(hydroxymethyl)phenyl]phenyl]ethyl]carbamoyl]-4-methyl-phenyl]piperazine-1-carboxylate

Using General Procedure 2 with tert-butyl 4-[3-[[(1R)-1-(3-bromophenyl)ethyl]carbamoyl]-4-methyl-phenyl]piperazine-1-carboxylate (381 mg, 758 μmol)—prepared in a similar manner to tert-Butyl 4-[4-methyl-3-[[(1R)-1-(1-naphthyl)ethyl]carbamoyl]phenyl]piperazine-1-carboxylate (Example 28)—with [3-(hydroxymethyl)phenyl]boronic acid (127 mg, 834 μmol) at 85° C. for 3.5 hours gave tert-butyl 4-[3-[[(1R)-1-[3-[3-(hydroxymethyl)phenyl]phenyl]ethyl]carbamoyl]-4-methyl-phenyl]piperazine-1-carboxylate (287 mg, 71%) as a white solid. LC-MS (Method B): R_{T}=3.86, m/z=528.8 [M−H]⁻.

### Step B: N-[(1R)-1-[3-[3-(Hydroxymethyl)phenyl]phenyl]ethyl]-2-methyl-5-piperazin-1-yl-benzamide

Using General Procedure 4 with tert-butyl 4-[3-[[(1R)-1-[3-[3-(hydroxymethyl)phenyl]phenyl]ethyl]carbamoyl]-4-methyl-phenyl]piperazine-1-carboxylate (287 mg, 542 μmol) gave N-[(1R)-1-[3-[3-(hydroxymethyl)phenyl]phenyl]ethyl]-2-methyl-5-piperazin-1-yl-benzamide (220 mg, 95%) as a white solid. LC-MS (Method B): R_{T}=3.08, m/z=428.7 [M−H]⁻.

### Step C: Benzyl 3-[4-[3-[[(1R)-1-[3-[3-(hydroxymethyl)phenyl]phenyl]ethyl]carbamoyl]-4-methyl-phenyl]piperazin-1-yl]propanoate

Benzyl acrylate (102 μL, 666 μmol,) was added to a solution of N-[(1R)-1-[3-[3-(hydroxymethyl)phenyl]phenyl]ethyl]-2-methyl-5-piperazin-1-yl-benzamide (220 mg, 512 μmol) in MeOH (15 mL) and the reaction mixture allowed to stir at RT for 2 hours. The solvent was removed in vacuo and purification by FCC (eluting with 0-50% MeOH in DCM) gave benzyl 3-[4-[3-[[(1R)-1-[3-[3-(hydroxymethyl)phenyl]phenyl]ethyl]carbamoyl]-4-methyl-phenyl]piperazin-1-yl]propanoate (253 mg, 83%) as an orange oily solid. LC-MS (Method B): R_{T}=3.84, m/z=591.0 [M−H]⁻.

### Step D: 3-[4-[3-[[(1R)-1-[3-[3-(Hydroxymethyl)phenyl]phenyl]ethyl]carbamoyl]-4-methyl-phenyl]piperazin-1-yl]propanoic Acid

Using General Procedure 5 with palladium hydroxide, 20% on carbon (30 mg) and benzyl 3-[4-[3-[[(1R)-1-[3-[3-(hydroxymethyl)phenyl]phenyl]ethyl]carbamoyl]-4-methyl-phenyl]piperazin-1-yl]propanoate (253 mg, 428 μmol) gave 3-[4-[3-[[(1R)-1-[3-[3-(hydroxymethyl)phenyl]phenyl]ethyl]carbamoyl]-4-methyl-phenyl]piperazin-1-yl]propanoic acid (70 mg, 31%) as a white crystalline solid. ¹H NMR (500 MHz, DMSO-d₆) δ 8.72 (d, J=8.0, 1H), 7.71 (m, 1H), 7.61 (m, 1H), 7.52 (m, 2H), 7.43 (td, J=7.5, 5.0, 2H), 7.37 (m, 1H), 7.33 (d, J=7.5, 1H), 7.07 (d, J=8.5, 1H), 6.91 (dd, J=8.5, 2.5, 1H), 6.86 (d, J=2.5, 1H), 5.18 (quin, J=7.0, 1H), 4.58 (s, 2H), 3.09 (m, 4H), 2.61 (t, J=7.0, 2H), 2.53 (m, 4H), 2.40 (t, J=7.0, 2H), 2.18 (s, 3H), 1.48 (d, J=7.0, 3H). LC-MS (Method B): R_{T}=0.39, m/z=500.8 [M−H]⁻.

### Example 171: 2-[4-[4-Methyl-3-[[(1R)-1-(1-naphthyl)ethyl]carbamoyl]phenyl]piperazin-1-yl]acetic Acid

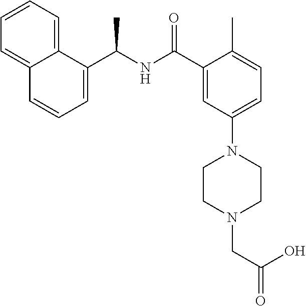 [SMILES: Cc1ccc(N2CCN(CC(=O)O)CC2)cc1C(=O)N[C@H](C)c1cccc2ccccc12]

### Step A: Benzyl 2-[4-[4-methyl-3-[[(1R)-1-(1-naphthyl)ethyl]carbamoyl]phenyl]piperazin-1-yl]acetate

2-Methyl-N-[(1R)-1-(1-naphthyl)ethyl]-5-piperazin-1-yl-benzamide (0.40 g, 1.07 mmol) (Example 108) and benzyl 2-bromoacetate (185 μL, 1.18 mmol) were added to DMF (25 mL). To this was added caesium carbonate (454 mg, 1.39 mmol) and the mixture was stirred at 50° C. for 1 hour. The mixture was quenched with water (100 mL), extracted with diethyl ether (2×75 mL), dried (MgSO₄) and solvent removed in vacuo to afford a yellow gum. Purification by FCC (eluting with 60% diethyl ether in petroleum ether) afforded benzyl 2-[4-[4-methyl-3-[[(1R)-1-(1-naphthyl)ethyl]carbamoyl]phenyl]piperazin-1-yl]acetate (502 mg, 90%) as a clear gum. LC-MS (Method B): R_{T}=4.19, m/z=521.0 [M−H]⁻.

### Step B: 2-[4-[4-Methyl-3-[[(1R)-1-(1-naphthyl)ethyl]carbamoyl]phenyl]piperazin-1-yl]acetic Acid

Using General Procedure 5 with benzyl 2-[4-[4-methyl-3-[[(1R)-1-(1-naphthyl)ethyl]carbamoyl]phenyl]piperazin-1-yl]acetate (500 mg, 959 μmol) and palladium, 10% on activated carbon powder (51 mg, 479 μmol) gave 2-[4-[4-methyl-3-[[(1R)-1-(1-naphthyl)ethyl]carbamoyl]phenyl]piperazin-1-yl]acetic acid (78 mg, 19%) as a white solid. ¹H NMR (500 MHz, CDCl₃) δ 8.22 (d, J=8.5, 1H), 7.86 (d, J=7.6, 1H), 7.80 (d, J=7.9, 1H), 7.58-7.53 (m, 2H), 7.53-7.48 (m, 1H), 7.48-7.42 (m, 1H), 7.05 (d, J=8.5, 1H), 6.84 (d, J=2.4, 1H), 6.82-6.76 (m, 1H), 6.17-6.08 (m, 2H), 3.35 (s, 2H), 3.24 (br s, 4H), 3.11 (br s, 4H), 2.30 (s, 3H), 1.78 (d, J=6.4, 3H). LC-MS (Method B): R_{T}=2.08, m/z=430.6 [M−H]⁻.

### Example 172: 4-[4-[4-Methyl-3-[[(1R)-1-(1-naphthyl)ethyl]carbamoyl]phenyl]piperazin-1-yl]butanoic Acid

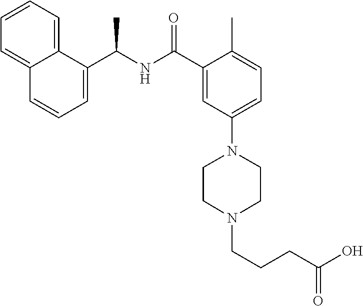 [SMILES: Cc1ccc(N2CCN(CCCC(=O)O)CC2)cc1C(=O)N[C@H](C)c1cccc2ccccc12]

### Step A: Benzyl 4-[4-[4-methyl-3-[[(1R)-1-(1-naphthyl)ethyl]carbamoyl]phenyl]piperazin-1-yl]butanoate

2-Methyl-N-[(1R)-1-(1-naphthyl)ethyl]-5-piperazin-1-yl-benzamide (274 mg, 733 μmol) (Example 108) and caesium carbonate (311 mg, 954 μmol) were added to DMF (10 mL). To this was added benzyl 4-bromobutanoate (219 mg, 852 μmol) and the mixture was stirred for 72 hours. The reaction was quenched with water (50 mL), extracted with diethyl ether (2×50 mL), dried (MgSO₄) and solvent Removed in vacuo to afford an orange gum. This was purified by FCC (eluting with diethyl ether and then 1:1 diethyl ether in ethyl acetate) to afford benzyl 4-[4-[4-methyl-3-[[(1R)-1-(1-naphthyl)ethyl]carbamoyl]phenyl]piperazin-1-yl]butanoate (258 mg, 64%) as a colourless gum. LC-MS (Method B): R_{T}=4.29, m/z=548.9 [M−H]⁻.

### Step B: 4-[4-[4-Methyl-3-[[(1R)-1-(1-naphthyl)ethyl]carbamoyl]phenyl]piperazin-1-yl]butanoic Acid

Using General Procedure 5 with benzyl 4-[4-[4-methyl-3-[[(1R)-1-(1-naphthyl)ethyl]carbamoyl]phenyl]piperazin-1-yl]butanoate (258 mg, 469 μmol) and palladium, 10% on activated carbon powder (25 mg, 235 μmol) gave 4-[4-[4-methyl-3-[[(1R)-1-(1-naphthyl)ethyl]carbamoyl]phenyl]piperazin-1-yl]butanoic acid (124 mg, 57%) as a white solid. ¹H NMR (500 MHz, CDCl₃) δ 8.26-8.21 (m, J=8.2, 1H), 7.88 (d, J=7.9, 1H), 7.85-7.78 (m, 1H), 7.60-7.54 (m, 2H), 7.54-7.45 (m, 2H), 7.05 (d, J=8.2, 1H), 6.86-6.78 (m, 2H), 6.13 (quin, J=7.1, 1H), 6.05-5.95 (m, 1H), 3.18 (br m, 4H), 2.81 (br m, 4H), 2.73-2.65 (m, 2H), 2.65-2.52 (m, 2H), 2.31 (s, 3H), 1.85 (br m, 2H), 1.82-1.77 (m, 3H). LC-MS (Method B): R_{T}=2.22, m/z=458.8 [M−H]⁻.

### Example 173: 3-(4-Methylpiperazine-1-carbonyl)-N-[(1R)-1-(1-naphthyl)ethyl]benzamide

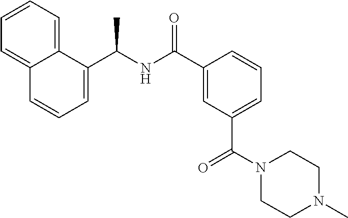 [SMILES: C[C@@H](NC(=O)c1cccc(C(=O)N2CCN(C)CC2)c1)c1cccc2ccccc12]

### Step A: 3-[[(1R)-1-(1-Naphthyl)ethyl]carbamoyl]benzoic Acid

Lithium hydroxide monohydrate (68 mg, 1.62 mmol) was added to a solution of methyl 3-[[(1R)-1-(1-naphthyl)ethyl]carbamoyl]benzoate (490 mg, 1.47 mmol)—prepared in a similar manner to N-[(1R)-1-(1-naphthyl)ethyl]-3-(1-piperidyl)benzamide (Example 1)—in THF (10 mL) and water (10 mL) and the reaction mixture stirred at 60° C. for 6 hours. The reaction mixture was cooled to RT and water (70 mL) and diethyl ether (100 mL) added and the phases separated. 2N HCl was added to the aqueous phase, resulting in formation of a precipitate. This material was filtered and dried under vacuum to give 3-[[(1R)-1-(1-naphthyl)ethyl]carbamoyl]benzoic acid (325 mg, 66%) as a white solid. ¹H NMR (500 MHz, DMSO-d₆) δ 13.19 (s, 1H), 9.21 (d, J=8.0, 1H), 8.49 (s, 1H), 8.21 (d, J=8.0, 1H), 8.14 (d, J=8.0, 1H), 8.08 (d, J=8.0, 1H), 7.96 (d, J=8.0, 1H), 7.84 (d, J=8.0, 1H), 7.65 (d, J=7.0, 1H), 7.62-7.57 (m, 2H), 7.55-7.50 (m, 2H), 5.98 (quin, J=7.0, 1H), 1.64 (d, J=7.0, 3H). LC-MS (Method B): R_{T}=1.90, m/z=318.6 [M−H]⁻.

### Step B: 3-(4-Methylpiperazine-1-carbonyl)-N-[(1R)-1-(1-naphthyl)ethyl]benzamide

Using General Procedure 1 with 3-[[(1R)-1-(1-naphthyl)ethyl]carbamoyl]benzoic acid (71 mg, 222 μmol) and 1-methylpiperazine (25 mg, 245 μmol) gave 3-(4-methylpiperazine-1-carbonyl)-N-[(1R)-1-(1-naphthyl)ethyl]benzamide (75 mg, 80%) as a white solid. ¹H NMR (500 MHz, DMSO-d₆) δ 9.08 (d, J=7.5, 1H), 8.21 (d, J=8.5, 1H), 7.99-7.95 (m, 2H), 7.92 (m, 1H), 7.84 (d, J=8.5, 1H), 7.64 (d, J=7.0, 1H), 7.60-7.50 (m, 5H), 5.97 (quin, J=7.0, 1H), 3.63 (m, 2H), 3.40 (m, 2H), 2.36 (m, 2H), 2.26 (m, 2H), 2.19 (s, 3H), 1.63 (d, J=7.0, 3H). LC-MS (Method B): R_{T}=3.12, m/z=400.8 [M−H]⁻.

### Further Examples

The following examples were prepared in a similar manner to 3-(4-methylpiperazine-1-carbonyl)-N-[(1R)-1-(1-naphthyl)ethyl]benzamide (Example 173), using the required commercially available amine in Step B.

| Example | Structure | Name and Analytical Data |
|---|---|---|
| 174 | 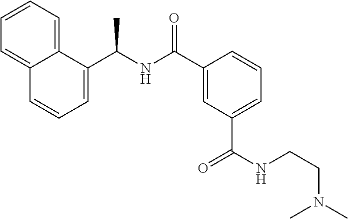 [SMILES: C[C@@H](NC(=O)c1cccc(C(=O)NCCN(C)C)c1)c1cccc2ccccc12] | N1-[2-(Dimethylamino)ethyl]-N3-[(1R)-1-(1- naphthyl)ethyl]benzene-1,3-dicarboxamide ¹H NMR (500 MHz, DMSO-d₆) δ 9.17 (d, J = 8.0, 1H), 8.56 (t, J = 5.5, 1H), 8.40 (t, J = 1.5, 1H), 8.29 (d, J = 8.5, 1H), 8.09 (dt, J = 8.0, 1.5, 1H), 8.03 (m, 2H), 7.92 (d, J = 8.0, 1H), 7.73 (d, J = 7.0, 1H), 7.67-7.57 (m, 4H), 6.06 (quin, J = 7.0, 1H), 3.44 (q, J = 7.0, 2H), 2.47 (t, J = 7.0, 2H), 2.24 (s, 6H), 1.71 (d, J = 7.0, 3H). LC-MS (Method B): R_{T} = 3.22, m/z = 388.8 [M − H]⁻. |

### Example 175: 3-[1-[2-(Benzenesulfonyl)ethyl]-4-piperidyl]-N-[(1R)-1-(1-naphthyl)ethyl]benzamide

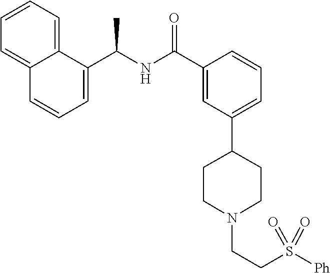 [SMILES: C[C@@H](NC(=O)c1cccc(C2CCN(CCS(=O)(=O)c3ccccc3)CC2)c1)c1cccc2ccccc12]

N-[(1R)-1-(1-Naphthyl)ethyl]-3-(4-piperidyl)benzamide hydrochloride salt (Example 6) (150 mg, 379 μmol) and phenyl vinyl sulfone (64 mg, 379 μmol) were added to DMF (5 mL). To this was added triethylamine (121 μL, 874 μmol) and the mixture was stirred for 2 hours. The reaction was quenched with water (40 mL) and stirred for 10 mins before being filtered and dried to give 3-[1-[2-(benzenesulfonyl)ethyl]-4-piperidyl]-N-[(1R)-1-(1-naphthyl)ethyl]benzamide (148 mg, 74%) as a white solid. ¹H NMR (500 MHz, CDCl₃) δ 8.18 (d, J=8.5, 1H), 7.94-7.91 (m, 2H), 7.89-7.86 (m, 1H), 7.86-7.80 (m, 1H), 7.65-7.58 (m, 2H), 7.57-7.47 (m, 7H), 7.30-7.23 (m, 4H), 6.27 (br d, J=8.2, 1H), 6.18-6.11 (m, 1H), 3.35-3.29 (m, 2H), 2.85-2.76 (m, 4H), 2.42 (tt, J=3.7, 12.2, 1H), 2.03 (dt, J=2.1, 11.6, 2H), 1.79 (d, J=6.7, 3H), 1.74-1.66 (m, 2H), 1.54-1.44 (m, 2H). LC-MS (Method B): R_{T}=4.07, m/z=526.0 [M−H]⁻.

### Further Examples

The following examples were prepared in a similar manner to 3-[1-[2-(benzenesulfonyl)ethyl]-4-piperidyl]-N-[(1R)-1-(1-naphthyl)ethyl]benzamide (Example 175), from the intermediate example stated.

| Example | | |
|---|---|---|
| (Intermediate) | Structure | Name and Analytical Data |
| 176 (Example 6) | 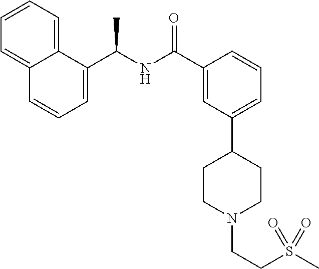 [SMILES: C[C@@H](NC(=O)c1cccc(C2CCN(CCS(C)(=O)=O)CC2)c1)c1cccc2ccccc12] | 3-[1-(2-Methylsulfonylethyl)-4-piperidyl]-N-[(1R)-1-(1- naphthyl)ethyl]benzamide ¹H NMR (500 MHz, CDCl₃) δ 8.18 (d, J = 8.5, 1H), 7.90-7.86 (m, 1H), 7.86-7.81 (m, 1H), 7.66 (s, 1H), 7.64-7.59 (m, 1H), 7.56-7.46 (m, 4H), 7.34-7.27 (m, 2H), 6.30 (br d, J = 7.6, 1H), 6.14 (quin, J = 7.1, 1H), 3.30 (t, J = 7.2, 2H), 3.05 (s, 3H), 3.04-3.00 (m, 2H), 2.92-2.86 (m, 2H), 2.54 (tt, J = 3.7, 12.2, 1H), 2.20-2.11 (m, 2H), 1.85 (m, 2H), 1.79 (d, J = 7.1, 3H), 1.77-1.65 (m, 2H). LC-MS (Method B): R_{T} = 3.86, m/z = 465.6 [M + H]⁺. |
| 177 (Example 128) | 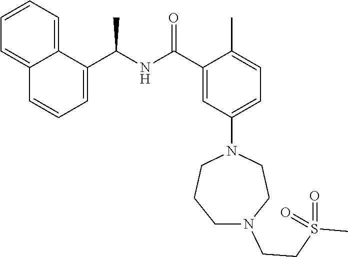 [SMILES: Cc1ccc(N2CCCN(CCS(C)(=O)=O)CC2)cc1C(=O)N[C@H](C)c1cccc2ccccc12] | 2-Methyl-5-[4-(2-methylsulfonylethyl)-1,4-diazepan-1-yl]-N-[(1R)-1- (1-naphthyl)ethyl]benzamide ¹H NMR (500 MHz, CDCl₃) δ 8.24 (d, J = 8.2, 1H), 7.88 (d, J = 7.9, 1H), 7.82 (d, J = 8.2, 1H), 7.60-7.54 (m, 2H), 7.54-7.45 (m, 2H), 6.99 (br d, J = 8.5, 1H), 6.61-6.56 (m, 2H), 6.12 (quin, J = 7.0, 1H), 6.01 (br d, J = 8.5, 1H), 3.51-3.36 (m, 4H), 3.17-2.55 (m, 11H), 2.27 (s, 3H), 2.00-1.84 (m, 2H), 1.80 (d, J = 7.0, 3H). LC-MS (Method B): R_{T} = 3.76, m/z = 494.7 [M + H]⁺. |

### Example 178: 2-Methyl-N-[(1R)-1-(1-naphthyl)ethyl]-5-(4-sulfamoyl-1,4-diazepan-1-yl)benzamide

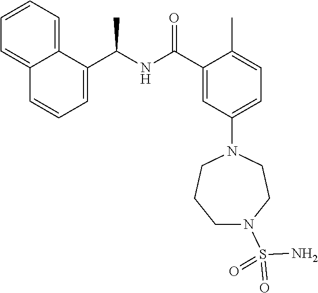 [SMILES: Cc1ccc(N2CCCN(S(N)(=O)=O)CC2)cc1C(=O)N[C@H](C)c1cccc2ccccc12]

### Step A: Benzyl N-[[4-[4-methyl-3-[[(1R)-1-(1-naphthyl)ethyl]carbamoyl]phenyl]-1,4-diazepan-1-yl]sulfonyl] carbamate

5-(1,4-Diazepan-1-yl)-2-methyl-N-[(1R)-1-(1-naphthyl)ethyl]benzamide (84 mg, 216 μmol) (Example 128) was added to DCM (10 mL), and to this was added triethylamine (45 μL, 325 μmol), benzyl N-chlorosulfonylcarbamate (54 mg, 216 μmol) and the mixture was stirred for 20 mins. The reaction was quenched with sat aq. NH₄Cl (20 mL) and water (10 ml), extracted with DCM (40 mL), dried (MgSO₄), filtered and solvent evaporated to afford benzyl N-[[4-[4-methyl-3-[[(1R)-1-(1-naphthyl)ethyl]carbamoyl]phenyl]-1,4-diazepan-1-yl]sulfonyl]carbamate (120 mg, 92%) as a yellow foam. This was used directly in Step B.

### Step B: 2-Methyl-N-[(1R)-1-(1-naphthyl)ethyl]-5-(4-sulfamoyl-1,4-diazepan-1-yl)benzamide

Using General Procedure 5 with benzyl N-[[4-[4-methyl-3-[[(1R)-1-(1-naphthyl)ethyl]carbamoyl]phenyl]-1,4-diazepan-1-yl]sulfonyl] carbamate (101 mg, 168 μmol) and palladium, 10% on activated carbon powder (1.8 mg, 16.8 μmol) gave 2-methyl-N-[(1R)-1-(1-naphthyl)ethyl]-5-(4-sulfamoyl-1,4-diazepan-1-yl)benzamide (52 mg, 66%) as a white solid. ¹H NMR (500 MHz, CDCl₃) δ 8.24 (d, J=8.5, 1H), 7.88 (d, J=8.5, 1H), 7.81 (d, J=7.9, 1H), 7.59-7.45 (m, 4H), 7.00 (d, J=7.9, 1H), 6.63-6.57 (m, 2H), 6.11 (quin, J=7.2, 1H), 6.05-5.96 (m, 1H), 4.20 (s, 2H), 3.60-3.41 (m, 6H), 3.28-3.18 (m, 2H), 2.27 (s, 3H), 2.02-1.90 (m, 2H), 1.80 (d, J=7.2, 3H). LC-MS (Method B): R_{T}=3.52, m/z=465.8 [M−H]⁻.

### Further Examples

The following examples were prepared in a similar manner to 2-methyl-N-[(1R)-1-(1-naphthyl)ethyl]-5-(4-sulfamoyl-1,4-diazepan-1-yl)benzamide (Example 178), from the intermediate example stated.

| Example | | |
|---|---|---|
| (Intermediate) | Structure | Name and Analytical Data |
| 179 (Example 108) | 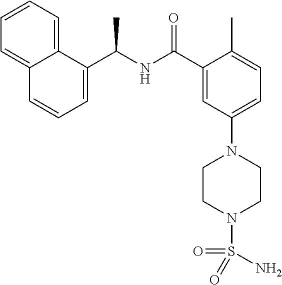 [SMILES: Cc1ccc(N2CCN(S(N)(=O)=O)CC2)cc1C(=O)N[C@H](C)c1cccc2ccccc12] | 2-Methyl-N-[(1R)-1-(1-naphthyl)ethyl]-5-(4-sulfamoylpiperazin-1-yl)benzamide ¹H NMR (500 MHz, DMSO-d₆) δ 8.82 (d, J = 7.9, 1H), 8.24 (d, J = 8.5, 1H), 7.96 (d, J = 7.9, 1H), 7.84 (d, J = 8.3, 1H), 7.65-7.57 (m, 2H), 7.57-7.50 (m, 2H), 7.08 (d, J = 8.5, 1H), 6.95 (dd, J = 2.6, 8.3, 1H), 6.92-6.89 (m, 1H), 6.86 (s, 2H), 5.90 (quin, J = 7.2, 1H), 3.25-3.15 (m, 4H), 3.14-3.04 (m, 4H), 2.20 (s, 3H), 1.59 (d, J = 7.2, 3H). LC-MS (Method B): R_{T} = 3.40, m/z = 451.8 [M − H]⁺. |

### Example 180: N-[(1R)-1-(3-Bromo-4-methoxy-phenyl)ethyl]-2-methyl-5-(4-methylpiperazin-1-yl)benzamide

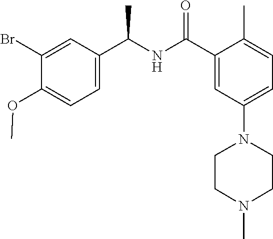 [SMILES: COc1ccc([C@@H](C)NC(=O)c2cc(N3CCN(C)CC3)ccc2C)cc1Br]

### Step A: (NE)-N-[(3-Bromo-4-methoxy-phenyl)methylene]-2-methyl-propane-2-sulfinamide

(S)-2-Methylpropane-2-sulfinamide (5.64 g, 46.5 mmol) and 3-bromo-4-methoxy-benzaldehyde (10.0 g, 46.5 mmol) were added to DCM (100 mL) and stirred until all solids dissolved. To this was added caesium carbonate (15.2 g, 46.5 mmol) and the mixture was stirred at reflux for 2 hours. The DCM was removed then the mixture diluted with water (100 mL), extracted with diethyl ether (2×100 mL), dried (MgSO₄) and solvent removed in vacuo to afford a yellow liquid. To this was added 30% diethyl ether in petroleum ether and the mixture was stirred for 1 hour to afford a solid which was filtered to afford (NE)-N-[(3-bromo-4-methoxy-phenyl)methylene]-2-methyl-propane-2-sulfinamide (11.4 g, 77%) as a white solid which was used directly in Step B.

### Step B: N-[(3-Bromo-4-methoxy-phenyl)methylene]-2-methyl-propane-2-sulfinamide

(NE)-N-[(3-bromo-4-methoxy-phenyl)methylene]-2-methyl-propane-2-sulfinamide (11.4 g, 35.8 mmol) was dissolved in THF (100 mL) and cooled to −30° C. under nitrogen. Once cooled methylmagnesium bromide solution (3.0 M, 14.3 mL) was slowly added to afford a yellow solution. The mixture was stirred at −30° C. for 1 hour before being allowed to warm up to RT over 90 mins. The mixture was carefully quenched with 0.5 N HCl (100 mL), extracted with diethyl ether (2×100 mL), dried (MgSO₄) and solvent removed in vacuo to afford a yellow gum. Purification on silica eluting with diethyl ether afforded N-[(1R)-1-(3-bromo-4-methoxy-phenyl)ethyl]-2-methyl-propane-2-sulfinamide (5.8 g, 48%) as a yellow gum which was used directly in Step C.

### Step C: (1R)-1-(3-Bromo-4-methoxy-phenyl)ethanamine Hydrochloride Salt

N-[(1R)-1-(3-Bromo-4-methoxy-phenyl)ethyl]-2-methyl-propane-2-sulfinamide (5.8 g, 17.4 mmol) was added to 1,4-dioxane (10 mL) to this was added 4.0 N HCl in 1,4-dioxane (17.4 mmol, 20 mL) and the mixture was stirred for 1 hour to afford a solid. The mixture was diluted with diethyl ether stirred and the solid filtered to afford (1R)-1-(3-bromo-4-methoxy-phenyl)ethanamine hydrochloride salt (3.82 g, 96%) as a white solid which was used directly in Step D.

### Step D: N-[(1R)-1-(3-bromo-4-methoxy-phenyl)ethyl]-2-methyl-5-(4-methylpiperazin-1-yl)benzamide

Using General Procedure 1 with (1R)-1-(3-bromo-4-methoxy-phenyl)ethanamine hydrochloride salt (1.00 g, 4.35 mmol) and 2-methyl-5-(4-methylpiperazin-1-yl)benzoic acid hydrochloride salt (1.32 g, 5.65 mmol) (Example 21, Step B) with purification by trituration from diethyl ether gave N-[(1R)-1-(3-bromo-4-methoxy-phenyl)ethyl]-2-methyl-5-(4-methylpiperazin-1-yl)benzamide (1.25 g, 65%) as a white solid. ¹H NMR (500 MHz, CDCl₃-d) δ 7.55 (d, J=2.1, 1H), 7.30 (dd, J=2.1, 8.5, 1H), 7.08 (d, J=8.2, 1H), 6.98-6.80 (m, 3H), 5.87 (br d, J=7.9, 1H), 5.25 (quin, J=7.2, 1H), 3.89 (s, 3), 3.22-3.12 (m, 4H), 2.62-2.51 (m, 4H), 2.35 (s, 3H), 2.33-2.29 (m, 3H), 1.56 (d, J=6.7, 3H). LC-MS (Method B): R_{T}=3.63, m/z=446.8 [M−H]⁻.

### Further Examples

The following examples were prepared in a similar manner to N-[(1R)-1-(3-bromo-4-methoxy-phenyl)ethyl]-2-methyl-5-(4-methylpiperazin-1-yl)benzamide (Example 180), using the required commercially available aldehyde in Step A and the required carboxylic acid—prepared in a similar manner to 2-methyl-5-(4-methylpiperazin-1-yl)benzoic acid hydrochloride salt (Example 21, Step B)—in Step D. Examples which did not provide solid material after Step D were treated with 2N HCl in diethyl ether, then concentrated and triturated with an appropriate solvent to give product as a hydrochloride salt.

| Example | Structure | Name and Analytical Data |
|---|---|---|
| 181 | 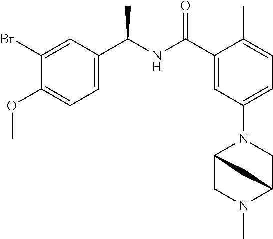 [SMILES: COc1ccc([C@@H](C)NC(=O)c2cc(N3C[C@H]4C[C@@H]3CN4C)ccc2C)cc1Br] | N-[(1R)-1-(3-Bromo-4-methoxy-phenyl)ethyl]-2-methyl-5-[(1R,4R)-5- methyl-2,5-diazabicyclo[2.2.1]heptan-2-yl]benzamide ¹H NMR (500 MHz, DMSO-d₆) δ 8.58 (d, J = 8.0, 1H), 7.60 (d, J = 2.0, 1H), 7.36 (dd, J = 8.5, 2.5, 1H), 7.08 (d, J = 8.5, 1H), 6.99 (d, J = 8.0, 1H), 6.55 (dd, J = 8.0, 2.5, 1H), 6.46 (d, J = 2.5, 1H), 5.05 (quin, J = 7.0, 1H), 4.26 (br s, 1H), 3.43 (br s, 1H), 3.32 (m, 3H), 3.14 (d, J = 9.0, 1H), 2.78 (dd, J = 9.0, 1.5, 1H), 2.26 (s, 3H), 2.13 (s, 3H), 1.86 (br d, J = 9.0, 1H), 1.75 (br d, J = 9.0, 1H), 1.39 (d, J = 7.0, 3H). LC-MS (Method B): R_{T} = 4.11, m/z = 456.8/458.8 [M − H]⁻. |
| 182 | 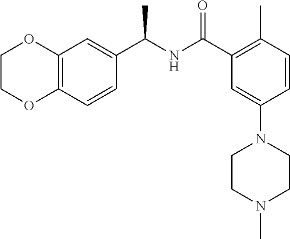 [SMILES: Cc1ccc(N2CCN(C)CC2)cc1C(=O)N[C@H](C)c1ccc2c(c1)OCCO2] | N-[(1R)-1-(2,3-Dihydro-1,4-benzodioxin-6-yl)ethyl]-2-methyl-5-(4- methylpiperazin-1-yl)benzamide ¹H NMR (500 MHz, DMSO-d₆) δ 8.52 (d, J = 8.2, 1H), 7.05 (d, J = 8.5, 1H), 6.93-6.86 (m, 2H), 6.85-6.77 (m, 3H), 5.03-4.97 (m, 1H), 4.24-4.19 (m, 4H), 3.16-3.05 (m, 4H), 2.49-2.42 (m, 4H), 2.24 (s, 3H), 2.16 (s, 3H), 1.37 (d, 3H). LC-MS (Method A): R_{T} = 2.89, m/z = 296.6 [M + H]⁺. |
| 183 | 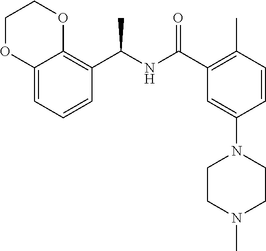 [SMILES: Cc1ccc(N2CCN(C)CC2)cc1C(=O)N[C@H](C)c1cccc2c1OCCO2] | N-[(1R)-1-(2,3-Dihydro-1,4-benzodioxin-5-yl)ethyl]-2-methyl-5-(4- methylpiperazin-1-yl)benzamide ¹H NMR (500 MHz, CDCl₃) δ 7.08 (d, J = 8.4, 1H), 6.94 (d, J = 2.7, 1H), 6.90-6.79 (m, 5H), 6.56-6.43 (m, 1H), 5.50-5.38 (m, 1H), 4.35-4.21 (m, 4H), 3.23-3.09 (m, 4H), 2.63-2.54 (m, 4H), 2.25 (s, 3H), 2.26 (s, 3H), 1.54 (d, 3H). LC-MS (Method A): R_{T} = 2.99, m/z not observed. |
| 184 | 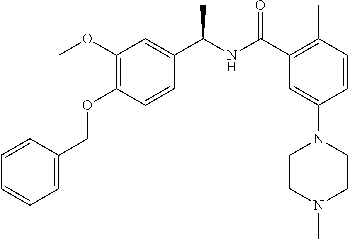 [SMILES: COc1cc([C@@H](C)NC(=O)c2cc(N3CCN(C)CC3)ccc2C)ccc1OCc1ccccc1] | N-[(1R)-1-(4-Benzyloxy-3-methoxy-phenyl)ethyl]-2-methyl-5-(4- methylpiperazin-1-yl)benzamide ¹H NMR (500 MHz, CDCl₃) δ 7.46-7.40 (m, 2H), 7.39-7.33 (m, 2H), 7.32-7.27 (m, 1H), 7.11-7.03 (m, 1H), 6.95-6.92 (m, 1H), 6.91-6.81 (m, 4H), 5.89 (br d, J = 8.1, 1H), 5.29-5.20 (m, 1H), 5.13 (s, 2H), 3.89 (s, 3H), 3.20-3.09 (m, 4H), 2.63-2.53 (m, 4H), 2.36 (s, 3H), 2.30 (s, 3H), 1.56 (d, J = 8.1, 3H). LC-MS (Method A): R_{T} = 4.08, m/z = 474.8 [M + H]⁺. |
| 185 | 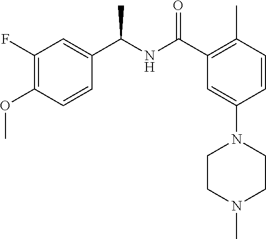 [SMILES: COc1ccc([C@@H](C)NC(=O)c2cc(N3CCN(C)CC3)ccc2C)cc1F] | N-[(1R)-1-(3-Fluoro-4-methoxy-phenyl)ethyl]-2-methyl-5-(4- methylpiperazin-1-yl)benzamide ¹H NMR (500 MHz, CDCl₃) δ 7.27-7.11 (m, 3H), 6.92-6.69 (m, 3H), 5.89 (br d, J = 8.1, 1H), 5.30-5.22 (m, 1H), 3.98 (s, 3H), 3.23-3.14 (m, 4H), 2.65-2.55 (m, 4H), 2.37 (s, 3H), 2.30 (s, 3H), 1.55 (d, J = 8.1, 3H). LC-MS (Method A): R_{T} = 3.19, m/z = 386.7 [M + H]⁺. |
| 186 | 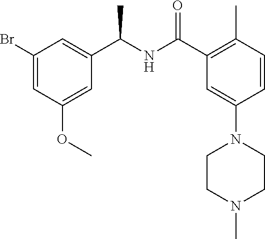 [SMILES: COc1cc(Br)cc([C@@H](C)NC(=O)c2cc(N3CCN(C)CC3)ccc2C)c1] | N-[(1R)-1-(3-Bromo-5-methoxy-phenyl)ethyl]-2-methyl-5-(4- methylpiperazin-1-yl)benzamide ¹H NMR (500 MHz, CDCl₃) δ 7.12-7.06 (m, 2H), 6.96 (t, J = 1.8, 1H), 6.92 (d, J = 2.6, 1H), 6.90-6.87 (m, 1H), 6.86-6.83 (m, 1H), 5.96-5.88 (m, 1H), 5.27-5.19 (m, 1H), 3.79 (s, 3H), 3.21-3.14 (m, 4H), 2.63-2.57 (m, 4H), 2.37 (s, 3H), 2.32 (s, 3H), 1.55 (d, J = 7.0, 3H). LC-MS (Method A): R_{T} = 3.58, m/z = 446.6/448.6 [M + H]⁺. |
| 187 | 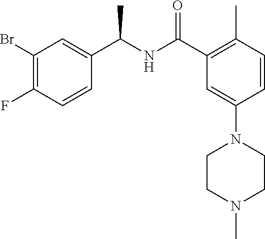 [SMILES: Cc1ccc(N2CCN(C)CC2)cc1C(=O)N[C@H](C)c1ccc(F)c(Br)c1] | N-[(1R)-1-(3-Bromo-4-fluoro-phenyl)ethyl]-2-methyl-5-(4-methylpiperazin- 1-yl)benzamide ¹H NMR (500 MHz, CDCl₃) δ 7.58-7.54 (m, 1H), 7.33-7.28 (m, 1H), 7.13-7.06 (m, 2H), 6.92-6.86 (m, 2H), 5.97-5.91 (m, 1H), 5.29-5.21 (m, 1H), 3.20-3.14 (m, 4H), 2.65-2.58 (m, 4H), 2.38 (s, 3H), 2.30 (s, 3H), 1.56 (d, J = 8.1, 3H). LC-MS (Method A): R_{T} = 3.92, m/z = 434.5/436.5 [M + H]⁺. |
| 188 | 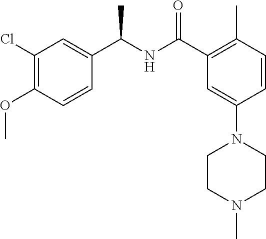 [SMILES: COc1ccc([C@@H](C)NC(=O)c2cc(N3CCN(C)CC3)ccc2C)cc1Cl] | N-[(1R)-1-(3-Chloro-4-methoxy-phenyl)ethyl]-2-methyl-5-(4- methylpiperazin-1-yl)benzamide ¹H NMR (500 MHz, CDCl₃) δ 7.38 (d, J = 2.1, 1H), 7.25-7.23 (m, 1H), 7.10-7.06 (m, 1H), 6.93-6.89 (m, 2H), 6.88-6.85 (m, 1H), 5.90-5.84 (m, 1H), 5.28-5.21 (m, 1H), 3.90 (s, 3H), 3.19-3.12 (m, 4H), 2.60-2.53 (m, 4H), 2.35 (s, 3H), 2.30 (s, 3H), 1.55 (d, J = 6.9, 3H). LC-MS (Method A): R_{T} = 3.83, m/z = 402.6 [M + H]⁺. |
| 189 | 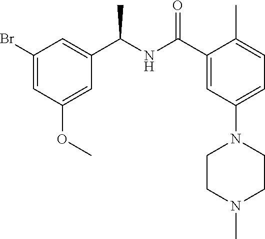 [SMILES: COc1cc(Br)cc([C@@H](C)NC(=O)c2cc(N3CCN(C)CC3)ccc2C)c1] | N-[(1R)-1-(3-Bromo-5-methoxy-phenyl)ethyl]-2-methyl-5-(4- methylpiperazin-1-yl)benzamide ¹H NMR (500 MHz, CDCl₃) δ 7.11-7.07 (m, 2H), 6.96 (t, J = 1.8, 1H), 6.92 (d, J = 2.4, 1H), 6.90-6.87 (m, 1H), 6.85 (s, 1H), 5.93 (br d, J = 7.9, 1H), 5.23 (quin, J = 7.2, 1H), 3.79 (s, 3H), 3.21-3.13 (m, 4H), 2.59-2.54 (m, 4H), 2.35 (s, 3H), 2.31 (S, 3H), 1.54 (d, J = 6.7, 3H). LC-MS (Method B): R_{T} = 3.72 m/z = 446.8 [M − H]⁻. |
| 190 | 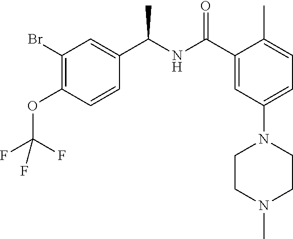 [SMILES: Cc1ccc(N2CCN(C)CC2)cc1C(=O)N[C@H](C)c1ccc(OC(F)(F)F)c(Br)c1] | N-[(1R)-1-[3-Bromo-4-(trifluoromethoxy)phenyl]ethyl]-2-methyl-5-(4- methylpiperazin-1-yl)benzamide ¹H NMR (500 MHz, CDCl₃) δ 7.65 (d, J = 1.8, 1H), 7.36 (dd, J = 2.0, 8.4, 1H), 7.32-7.27 (m, 1H), 7.10 (d, J = 8.2, 1H), 6.98-6.83 (m, 2H), 5.93 (br d, J = 7.3, 1H), 5.28 (quin, J = 7.2, 1H), 3.23-3.12 (m, 4H), 2.63-2.51 (m, 4H), 2.35 (s, 3H), 2.33-2.29 (m, 3H), 1.61-1.54 (m, 3H). LC-MS (Method B): R_{T} = 4.14 m/z = 500.8 [M − H]⁻. |
| 191 | 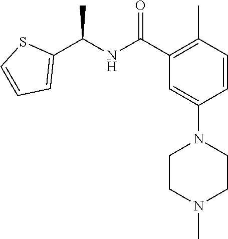 [SMILES: Cc1ccc(N2CCN(C)CC2)cc1C(=O)N[C@H](C)c1cccs1] | 2-Methyl-5-(4-methylpiperazin-1-yl)-N-[(1R)-1-(2-thienyl)ethyl]benzamide hydrochloride salt ¹H NMR (500 MHz, DMSO-d₆) δ 10.92 (br s, 1H), 8.60 (d, J = 8.5, 1H), 7.49 (dd, J = 5.0, 3.0, 1H), 7.34 (m, 1H), 7.15 (d, J = 5.0, 1H), 7.12 (d, J = 8.0, 1H), 6.98 (dd, J = 8.0, 3.0, 1H), 6.92 (d, J = 3.0, 1H), 5.22 (quin, J = 7.0, 1H), 3.78 (br d, J = 12.0, 2H), 3.48 (br d, J = 11.5, 2H), 3.15-3.05 (m, 4H), 2.80 (d, J = 5.0, 3H), 2.22 (s, 3H), 1.46 (d, J = 7.0, 3H). LC-MS (Method B): R_{T} = 3.18, m/z = 342.7 [M − H]⁻. |
| 192 | 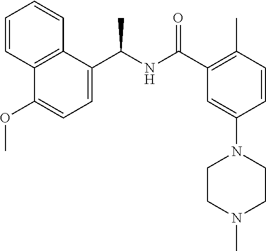 [SMILES: COc1ccc([C@@H](C)NC(=O)c2cc(N3CCN(C)CC3)ccc2C)c2ccccc12] | N-[(1R)-1-(4-Methoxy-1-naphthyl)ethyl]-2-methyl-5-(4-methylpiperazin-1- yl)benzamide ¹H NMR (500 MHz, DMSO-d₆) δ 8.69 (d, J = 8.0, 1H), 8.22 (d, J = 8.5, 1H), 8.18 (d, J = 8.5, 1H), 7.60 (m, 1H), 7.54-7.51 (m, 2H), 7.04 (d, J = 8.5, 1H), 6.98 (d, J = 8.0, 1H), 6.89 (dd, J = 8.0, 3.0, 1H), 6.83 (d, J = 3.0, 1H), 5.81 (quin, J = 7.0, 1H), 3.97 (s, 3H), 3.08 (m, 4H), 2.46 (m, 4H), 2.23 (s, 3H), 2.18 (s, 3H), 1.57 (d, J = 7.0, 3H). LC-MS (Method B): R_{T} = 3.78, m/z = 416.9 [M − H]⁻. |
| 193 | 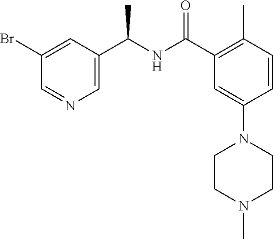 [SMILES: Cc1ccc(N2CCN(C)CC2)cc1C(=O)N[C@H](C)c1cncc(Br)c1] | N-[(1R)-1-(5-Bromo-3-pyridyl)ethyl]-2-methyl-5-(4-methylpiperazin-1- yl)benzamide ¹H NMR (500 MHz, DMSO-d₆) δ 8.79 (d, J = 7.5, 1H), 8.68-8.66 (m, 2H), 8.12 (t, J = 2.0, 1H), 7.17 (d, J = 8.5, 1H), 7.03 (dd, J = 8.5, 2.5, 1H), 6.96 (d, J = 2.5, 1H), 5.20 (quin, J = 7.0, 1H), 3.04 (m, 4H), 2.57 (br s, 3H), 2.22 (s, 3H), 1.53 (d, J = 7.0, 3H). LC-MS (Method B): R_{T} = 3.27, m/z = 415.8/417.8 [M − H]⁻. |
| 194 | 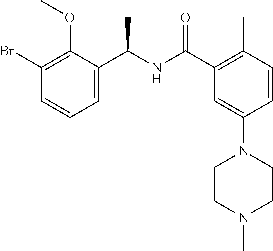 [SMILES: COc1c(Br)cccc1[C@@H](C)NC(=O)c1cc(N2CCN(C)CC2)ccc1C] | N-[(1R)-1-(3-Bromo-2-methoxy-phenyl)ethyl]-2-methyl-5-(4- methylpiperazin-1-yl)benzamide ¹H NMR (500 MHz, DMSO-d₆) δ 8.75 (d, J = 8.0, 1H), 7.52 (dd, J = 8.0, 1.5, 1H), 7.47 (dd, J = 8.0, 1.5, 1H), 7.13-7.08 (m, 2H), 6.94 (dd, J = 8.5, 2.5, 1H), 6.87 (d, J = 2.5, 1H), 5.41 (quin, J = 7.0, 1H), 3.91 (s, 3H), 3.19 (br s, 4H), 2.75 (br s, 4H), 2.44 (br s, 3H), 2.16 (s, 3H), 1.37 (d, J = 7.0, 3H). LC- MS (Method B): R_{T} = 3.73, m/z = 444.8/446.8 [M − H]⁻. |
| 195 | 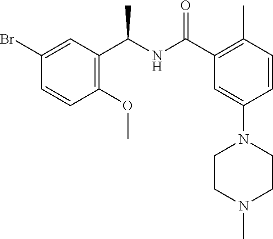 [SMILES: COc1ccc(Br)cc1[C@@H](C)NC(=O)c1cc(N2CCN(C)CC2)ccc1C] | N-[(1R)-1-(5-Bromo-2-methoxy-phenyl)ethyl]-2-methyl-5-(4- methylpiperazin-1-yl)benzamide ¹H NMR (500 MHz, DMSO-d₆) δ 8.61 (d, J = 8.5, 1H), 7.50 (d, J = 2.5, 1H), 7.39 (dd, J = 8.5, 2.5, 1H), 7.09 (d, J = 8.5, 1H), 6.98 (d, J = 8.5, 1H), 6.93 (dd, J = 8.5, 2.5, 1H), 6.86 (d, J = 2.5, 1H), 5.34 (quin, J = 7.0, 1H), 3.85 (s, 3H), 3.12 (m, 4H), 2.48 (m, 4H), 2.24 (s, 3H), 2.17 (s, 3H), 1.33 (d, J = 7.0, 3H). LC-MS (Method B): R_{T} = 3.94, m/z = 444.8/446.8 [M − H]⁻. |
| 196 | 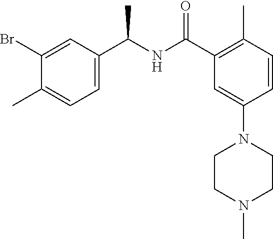 [SMILES: Cc1ccc([C@@H](C)NC(=O)c2cc(N3CCN(C)CC3)ccc2C)cc1Br] | N-[(1R)-1-(3-Bromo-4-methyl-phenyl)ethyl]-2-methyl-5-(4- methylpiperazin-1-yl)benzamide ¹H NMR (500 MHz, DMSO-d₆) δ 8.64 (d, J = 8.0, 1H), 7.61 (br s, 1H), 7.33-7.28 (m, 2H), 7.07 (d, J = 8.5, 1H), 6.92 (dd, J = 8.5, 2.5, 1H), 6.85 (d, J = 2.5, 1H), 5.06 (quin, J = 7.0, 1H), 3.14 (m, 4H), 2.58 (m, 4H), 2.32 (br s, 6H), 2.16 (s, 3H), 1.40 (d, J = 7.0, 3H). LC-MS (Method B): R_{T} = 4.07, m/z = 428.8/430.8 [M − H]⁻. |
| 197 | 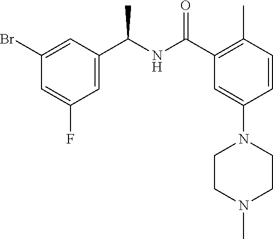 [SMILES: Cc1ccc(N2CCN(C)CC2)cc1C(=O)N[C@H](C)c1cc(F)cc(Br)c1] | N-[(1R)-1-(3-Bromo-5-fluoro-phenyl)ethyl]-2-methyl-5-(4-methylpiperazin- 1-yl)benzamide ¹H NMR (500 MHz, DMSO-d₆) δ 8.69 (d, J = 8.0, 1H), 7.47 (br s, 1H), 7.42 (m, 1H), 7.27 (br d, J = 10.0, 1H), 7.07 (d, J = 8.5, 1H), 6.92 (dd, J = 8.5, 2.5, 1H), 6.86 (d, J = 2.5, 1H), 5.09 (quin, J = 7.0, 1H), 3.12 (m, 4H), 2.48 (m, 4H), 2.24 (s, 3H), 2.16 (s, 3H), 1.42 (d, J = 7.0, 3H). LC-MS (Method B): R_{T} = 3.80, m/z = 432.6/434.6 [M − H]⁻. |
| 198 | 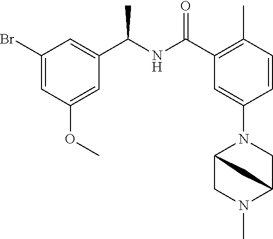 [SMILES: COc1cc(Br)cc([C@@H](C)NC(=O)c2cc(N3C[C@H]4C[C@@H]3CN4C)ccc2C)c1] | N-[(1R)-1-(3-Bromo-5-methoxy-phenyl)ethyl]-2-methyl-5-[(1R,4R)-5- methyl-2,5-diazabicyclo[2.2.1]heptan-2-yl]benzamide ¹H NMR (500 MHz, DMSO-d₆) δ 8.62 (d, J = 8.0, 1H), 7.17 (br s, 1H), 7.02-6.98 (m, 3H), 6.55 (dd, J = 8.5, 2.5, 1H), 6.48 (d, J = 2.5, 1H), 5.05 (quin, J = 7.0, 1H), 4.25 (br s, 1H), 3.78 (s, 3H), 3.40 (br s, 1H), 3.30 (dd, J = 8.5, 2.0, 1H), 3.16 (m, 1H), 2.76 (dd, J = 9.0, 2.0, 1H), 2.46 (d, J = 9.5, 1H), 2.24 (s, 3H), 2.14 (s, 3H), 1.85 (br d, J = 9.0, 1H), 1.74 (br d, J = 9.0, 1H), 1.40 (d, J = 7.0, 3H). LC-MS (Method B): R_{T} = 4.13, m/z = 456.7/458.7 [M − H]⁻. |
| 199 | 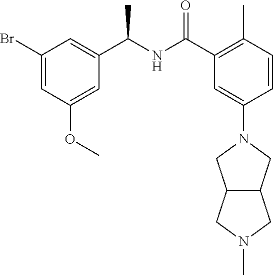 [SMILES: COc1cc(Br)cc([C@@H](C)NC(=O)c2cc(N3CC4CN(C)CC4C3)ccc2C)c1] | N-[(1R)-1-(3-Bromo-5-methoxy-phenyl)ethyl]-2-methyl-5-(2-methyl- 1,3,3a,4,6,6a-hexahydropyrrolo[3,4-c]pyrrol-5-yl)benzamide ¹H NMR (500 MHz, DMSO-d₆) δ 8.62 (d, J = 8.0, 1H), 7.17 (s, 1H), 7.02 (m, 2H), 6.98 (br s, 1H), 6.62 (dd, J = 8.5, 2.5, 1H), 6.57 (d, J = 2.5, 1H), 5.05 (quin, J = 7.0, 1H), 3.76 (s, 3H), 3.05 (m, 2H), 2.87 (m, 2H), 2.55 (m, 2H), 2.38 (dd, J = 9.0, 2.5, 2H), 2.21 (s, 3H), 2.15 (s, 3H), 1.40 (d, J = 7.0, 3H). LC-MS (Method B): R_{T} = 4.42, m/z = 470.8/472.8 [M − H]⁻. |
| 200 | 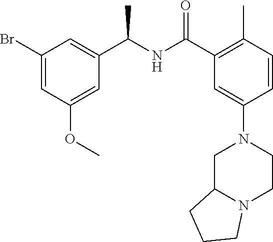 [SMILES: COc1cc(Br)cc([C@@H](C)NC(=O)c2cc(N3CCN4CCCC4C3)ccc2C)c1] | 5-(3,4,6,7,8,8a-Hexahydro-1H-pyrrolo[1,2a]pyrazin-2-yl)-N-[(1R)-1-(3- bromo-5-methoxy-phenyl)ethyl]-2-methyl-benzamide ¹H NMR (500 MHz, DMSO-d₆) δ 8.65 (d, J = 8.0, 1H), 7.17 (br s, 1H), 7.07 (d, J = 8.0, 1H), 7.02 (br s, 1H), 6.98 (br s, 1H), 6.94 (dd, J = 8.5, 2.5, 1H), 6.86 (d, J = 2.5, 1H), 5.05 (quin, J = 7.0, 1H), 3.78 (s, 3H), 3.74 (br d, J = 10.5, 1H), 3.60 (br d, J = 10.5, 1H), 3.08 (m, 2H), 2.74 (m, 1H), 2.32 (m, 1H), 2.29 (m, 1H), 2.15 (s, 3H), 2.14 (m, 1H), 1.86 (m, 1H), 1.73 (m, 2H), 1.41 (d, J = 7.0, 3H), 1.40 (m, 1H). LC-MS (Method B): R_{T} = 3.93, m/z = 470.8/472.8 [M − H]⁻. |
| 201 | 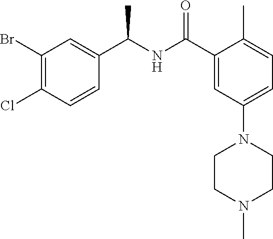 [SMILES: Cc1ccc(N2CCN(C)CC2)cc1C(=O)N[C@H](C)c1ccc(Cl)c(Br)c1] | N-[(1R)-1-(3-Bromo-4-chloro-phenyl)ethyl]-2-methyl-5-(4-methylpiperazin- 1-yl)benzamide ¹H NMR (500 MHz, CDCl₃) δ 7.63 (s, 1H), 7.43 (d, J = 8.2, 1H), 7.32-7.21 (m, 1H), 7.09 (br d, J = 8.2, 1H), 6.98-6.82 (m, 2H), 5.94 (br d, J = 7.3, 1H), 5.24 (quin, J = 7.1, 1H), 3.24-3.11 (m, 4H), 2.63-2.55 (m, 4H), 2.36 (s, 3H), 2.33-2.28 (m, 3H), 1.55-1.55 (m, 3H). LC-MS (Method B): R_{T} = 3.86, m/z = 450.7 [M − H]⁻. |
| 202 | 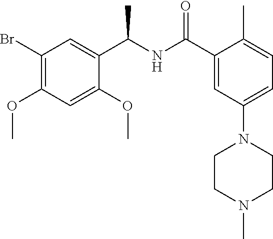 [SMILES: COc1cc(OC)c([C@@H](C)NC(=O)c2cc(N3CCN(C)CC3)ccc2C)cc1Br] | N-[(1R)-1-(5-Bromo-2,4-dimethoxy-phenyl)ethyl]-2-methyl-5-(4- methylpiperazin-1-yl)benzamide ¹H NMR (500 MHz, CDCl₃) δ 7.43-7.39 (m, 1H), 7.10-7.06 (m, 1H), 6.92 (d, J = 2.6, 1H), 6.89-6.84 (m, 1H), 6.50-6.46 (m, 1H), 6.41-6.35 (m, 1H), 5.41-5.33 (m, 1H), 3.90 (s, 3H), 3.87 (s, 3H), 3.22-3.14 (m, 4H), 2.64-2.55 (m, 4H), 2.37 (s, 3H), 2.30 (s, 3H), 1.51 (d, J = 6.9, 3H). LC-MS (Method A): R_{T} = 3.97, m/z = 478.6 [M + H]⁺. |

### Example 203: N-[(1R)-1-(4-Methoxy-3-phenyl-phenyl)ethyl]-2-methyl-5-(4-methylpiperazin-1-yl)benzamide

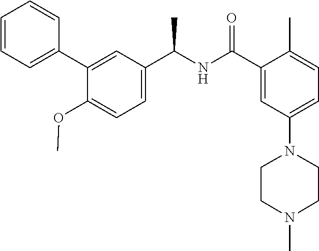 [SMILES: COc1ccc([C@@H](C)NC(=O)c2cc(N3CCN(C)CC3)ccc2C)cc1-c1ccccc1]

Using General Procedure 2 with 4,4,5,5-tetramethyl-2-phenyl-1,3,2-dioxaborolane (75 mg, 369 μmol) and N-[(1R)-1-(3-bromo-4-methoxy-phenyl)ethyl]-2-methyl-5-(4-methylpiperazin-1-yl)benzamide (Example 180) (150 mg, 336 μmol) at 50° C. for 30 mins with purification by FCC (eluting with 0-50% MeOH in ethyl acetate) gave N-[(1R)-1-(4-methoxy-3-phenyl-phenyl)ethyl]-2-methyl-5-(4-methylpiperazin-1-yl)benzamide (120 mg, 81%) as a white foam. ¹H NMR (500 MHz, CDCl₃) δ 7.56-7.46 (m, 2H), 7.43-7.38 (m, 2H), 7.36-7.30 (m, 3H), 7.07 (d, J=8.5, 1H), 6.97 (d, J=8.2, 1H), 6.92 (d, J=2.7, 1H), 6.90-6.83 (m, 1H), 5.91 (br d, J=7.9, 1H), 5.32 (quin, J=7.2, 1H), 3.81 (s, 3H), 3.18-3.12 (m, 4H), 2.59-2.52 (m, 4H), 2.34 (s, 3H), 2.32 (s, 3H), 1.60 (d, J=6.7, 3H). LC-MS (Method B): R_{T}=3.88, m/z=442.9 [M−H]⁻.

### Further Examples

The following examples were prepared in a similar manner to N-[(1R)-1-(4-methoxy-3-phenyl-phenyl)ethyl]-2-methyl-5-(4-methylpiperazin-1-yl)benzamide (Example 203), using the required commercially available boronic acid or ester, and the intermediate example aromatic bromide stated.

| Example | | |
|---|---|---|
| (Intermediate) | Structure | Name and Analytical Data |
| 204 (Example 180) | 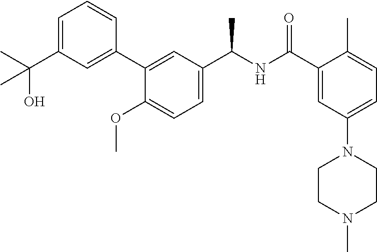 [SMILES: COc1ccc([C@@H](C)NC(=O)c2cc(N3CCN(C)CC3)ccc2C)cc1-c1cccc(C(C)(C)O)c1] | N-[(1R)-1-[3-[3-(1-Hydroxy-1-methyl-ethyl)phenyl]-4-methoxy- phenyl]ethyl]-2-methyl-5-(4-methylpiperazin-1-yl)benzamide ¹H NMR (500 MHz, CDCl₃) δ 7.72-7.56 (m, 1H), 7.47 (td, J = 1.7, 7.2, 1H), 7.42-7.31 (m, 3H), 7.08 (d, J = 8.5, 1H), 7.00-6.94 (m, 1H), 6.81 (d, J = 2.7, 1H), 6.75-6.70 (m, 1H), 5.93 (br d, J = 8.2, 2H), 5.36-5.29 (m, 1H), 3.81 (s, 3H), 3.17-3.07 (m, 4H), 2.58-2.50 (m, 4H), 2.33 (d, J = 4.3, 6H), 1.65-1.58 (m, 9H) (OH missing). LC-MS (Method B): R_{T} = 3.55, m/z = 501.0 [M − H]⁻. |
| 205 (Example 180) | 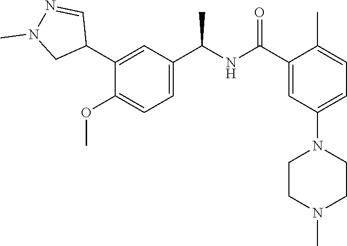 | N-[(1R)-1-[4-Methoxy-3-(1-methylpyrazol-4-yl)phenyl]ethyl]-2- methyl-5-(4-methylpiperazin-1-yl)benzamide ¹H NMR (500 MHz, CDCl₃) δ 7.86 (s, 1H), 7.83 (s, 1H), 7.51 (d, J = 2.1, 1H), 7.22 (dd, J = 2.4, 8.5, 1H), 7.08 (d, J = 8.2, 1H), 6.90-6.84 (m, 2H), 6.82-6.78 (m, 1H), 5.91 (br d, J = 7.9, 1H), 5.34-5.27 (m, 1H), 3.95 (s, 3H), 3.90 (s, 3H), 3.19-3.12 (m, 4H), 2.60-2.51 (m, 4H), 2.34 (s, 3H), 2.32 (s, 3H), 1.61 (br d, J = 6.7, 3H). LC-MS (Method B): R_{T} = 3.26, m/z = 446.9 [M − H]⁻. |
| 206 (Example 180) | 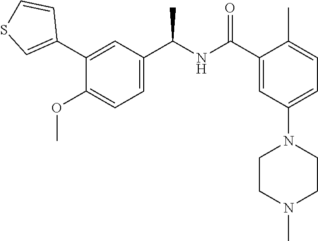 [SMILES: COc1ccc([C@@H](C)NC(=O)c2cc(N3CCN(C)CC3)ccc2C)cc1-c1ccsc1] | N-[(1R)-1-[4-Methoxy-3-(3-thienyl)phenyl]ethyl]-2-methyl-5-(4- methylpiperazin-1-yl)benzamide ¹H NMR (500 MHz, CDCl₃) δ 7.61-7.57 (m, 1H), 7.49 (d, J = 2.4, 1H), 7.42 (dd, J = 1.2, 5.2, 1H), 7.34 (dd, J = 3.1, 5.2, 1H), 7.29 (dd, J = 2.4, 8.5, 1H), 7.08 (d, J = 8.5, 1H), 6.96 (d, J = 8.5, 1H), 6.92 (d, J = 2.4, 1H), 6.87 (dd, J = 2.7, 8.5, 1H), 5.91 (br d, J = 7.9, 1H), 5.31 (quin, J = 7.2, 1H), 3.87 (s, 3H), 3.19-3.12 (m, 4H), 2.59-2.52 (m, 4H), 2.34 (s, 3H), 2.33-2.31 (m, 3H), 1.60 (d, J = 7.0, 3H). LC-MS (Method B): R_{T} = 3.85, m/z = 448.9 [M − H]⁻. |
| 207 (Example 180) | 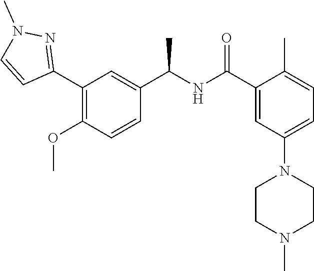 [SMILES: COc1ccc([C@@H](C)NC(=O)c2cc(N3CCN(C)CC3)ccc2C)cc1-c1ccn(C)n1] | N-[(1R)-1-[4-Methoxy-3-(1-methylpyrazol-3-yl)phenyl]ethyl]-2- methyl-5-(4-methylpiperazin-1-yl)benzamide ¹H NMR (500 MHz, DMSO-d₆) δ 8.68 (br d, J = 4.3, 1H), 7.91 (d, J = 2.4, 1H), 7.70 (br s, 1H), 7.30 (br d, J = 8.5, 1H), 7.12 (d, J = 8.2, 1H), 7.08-7.02 (m, 1H), 6.98 (dd, J = 2.4, 8.2, 1H), 6.94-6.87 (m, 1H), 6.69 (d, J = 1.8, 1H), 5.18-4.97 (m, 1H), 3.89 (s, 3H), 3.84 (s, 3H), 3.82-3.73 (m, 2H), 3.47 (br d, J = 11.9, 2H), 3.23-2.97 (m, 4H), 2.80 (br d, J = 1.5, 3H), 2.20 (s, 3H), 1.44 (d, J = 7.0, 3H). LC-MS (Method B): R_{T} = 3.43, m/z = 447.0 [M − H]⁻. |
| 208 (Example 180) | 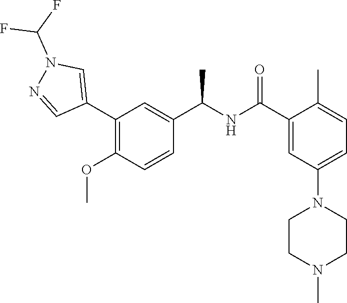 [SMILES: COc1ccc([C@@H](C)NC(=O)c2cc(N3CCN(C)CC3)ccc2C)cc1-c1cnn(C(F)F)c1] | N-[(1R)-1-[3-[1-(Difluoromethyl)pyrazol-4-yl]-4-methoxy- phenyl]ethyl]-2-methyl-5-(4-methylpiperazin-1-yl)benzamide ¹H NMR (500 MHz, DMSO-d₆) δ 8.61 (d, J = 8.2, 1H), 8.59-8.56 (m, 1H), 8.27 (s, 1H), 7.87 (t, J = 60; 1H), 7.75 (s, 1H), 7.31 (d, J = 8.9, 1H), 7.17-7.04 (m, 2H), 7.01-6.94 (m, 1H), 6.94-6.87 (m, 1H), 5.14 (quin, J = 7.3, 1H), 3.89 (s, 3H), 3.47 (br d, J = 11.9, 2H), 3.27-2.96 (m, 4H), 2.81 (d, J = 4.6, 3H), 2.25-2.10 (m, 3H), 1.53-1.36 (m, 3H). LC-MS (Method B): R_{T} = 3.66, m/z = 483.0 [M − H]⁻. |
| 209 (Example 180) | 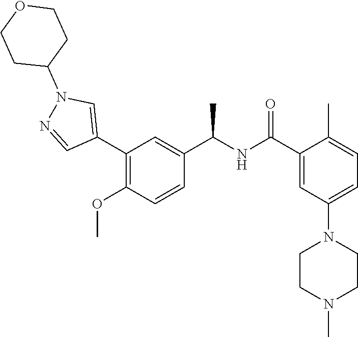 [SMILES: COc1ccc([C@@H](C)NC(=O)c2cc(N3CCN(C)CC3)ccc2C)cc1-c1cnn(C2CCOCC2)c1] | N-[(1R)-1-[4-Methoxy-3-(1-tetrahydropyran-4-ylpyrazol-3- yl)phenyl]ethyl]-2-methyl-5-(4-methylpiperazin-1-yl)benzamide ¹H NMR (500 MHz, CDCl₃) δ 7.90 (s, 2H), 7.54-7.50 (m, 1H), 7.22 (dd, J = 2.3, 8.4, 1H), 7.08 (d, J = 8.2, 1H), 6.96-6.90 (m, 2H), 6.87 (dd, J = 2.6, 8.4, 1H), 5.91 (br d, J = 8.2, 1H), 5.35-5.25 (m, 1H), 4.43-4.32 (m, 1H), 4.13 (td, J = 3.2, 11.6, 2H), 3.91 (s, 3H), 3.64-3.50 (m, 2H), 3.15 (br d, J = 4.9, 4H), 2.59-2.49 (m, 4H), 2.70 (s, 3H), 2.61 (s, 3H), 2.19-2.07 (m, 4H), 1.66-1.52 (m, 3H). LC-MS (Method B): R_{T} = 3.47, m/z = 517.1 [M − H]⁻. |
| 210 (Example 180) | 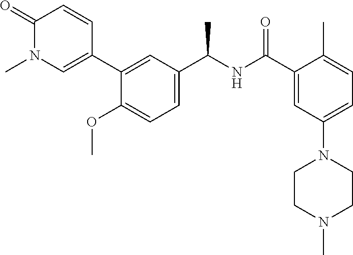 [SMILES: COc1ccc([C@@H](C)NC(=O)c2cc(N3CCN(C)CC3)ccc2C)cc1-c1ccc(=O)n(C)c1] | N-[(1R)-1-[4-Methoxy-3-(1-methyl-6-oxo-3- pyridyl)phenyl]ethyl]-2-methyl-5-(4-methylpiperazin-1- yl)benzamide ¹H NMR (500 MHz, DMSO-d₆) δ 8.57 (d, J = 8.0, 1H), 7.83 (d, J = 3.0, 1H), 7.60 (dd, J = 9.5, 2.5, 1H), 7.33-7.31 (m, 2H), 7.06 (d, J = 9.0, 2H), 6.90 (d, J = 8.0, 3.0, 1H), 6.83 (d, J = 3.0, 1H), 6.43 (d, J = 9.5, 1H), 5.10 (quin, J = 7.0, 1H), 3.78 (s, 3H), 3.49 (s, 3H), 3.09 (m, 4H), 2.43 (m, 4H), 2.22 (s, 3H), 2.17 (s, 3H), 1.43 (d, J = 7.0, 3H). LC-MS (Method B): R_{T} = 3.17, m/z = 475.9 [M + H]⁺. |
| 211 (Example 180) | 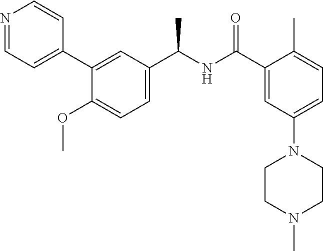 [SMILES: COc1ccc([C@@H](C)NC(=O)c2cc(N3CCN(C)CC3)ccc2C)cc1-c1ccncc1] | N-[(1R)-1-[4-Methoxy-3-(4-pyridyl)phenyl]ethyl]-2-methyl-5-(4- methylpiperazin-1-yl)benzamide ¹H NMR (500 MHz, DMSO-d₆) δ 8.62-8.59 (m, 3H), 7.51 (m, 2H), 7.44-7.42 (m, 2H), 7.14 (d, J = 9.0, 1H), 7.05 (d, J = 8.5, 1H), 6.90 (dd, J = 8.0, 2.5, 1H), 6.82 (d, J = 2.5, 1H), 5.13 (quin, J = 7.0, 1H), 3.79 (s, 3H), 3.09-3.07 (m, 4H), 2.44-2.42 (m, 4H), 2.22 (s, 3H), 2.17 (s, 3H), 1.44 (d, J = 7.0, 3H). LC-MS (Method B): R_{T} = 3.55, m/z = 443.9 [M − H]⁻. |
| 212 (Example 180) | 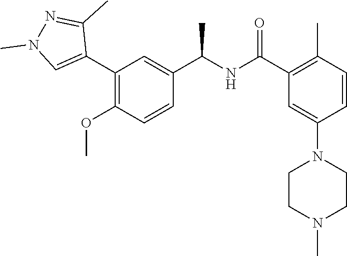 [SMILES: COc1ccc([C@@H](C)NC(=O)c2cc(N3CCN(C)CC3)ccc2C)cc1-c1cn(C)nc1C] | N-[(1R)-1-[3-(1,3-Dimethylpyrazol-4-yl)-4-methoxy- phenyl]ethyl]-2-methyl-5-(4-methylpiperazin-1-yl)benzamide ¹H NMR (500 MHz, DMSO-d₆) δ 8.58 (d, J = 8.5, 1H), 7.67 (s, 1H), 7.27 (d, J = 2.0, 1H), 7.24 (dd, J = 8.5, 2.0, 1H), 7.05 (d, J = 7.0, 1H), 7.00 (d, J = 8.5, 1H), 6.89 (dd, J = 8.5, 2.5, 1H), 6.82 (d, J = 2.5, 1H), 5.09 (quin, J = 7.0, 1H), 3.78 (s, 3H), 3.76 (s, 3H), 3.08 (m, 4H), 2.45-2.43 (m, 4H), 2.22 (s, 3H), 2.17 (s, 3H), 2.15 (s, 3H), 1.42 (d, J = 7.0, 3H). LC-MS (Method B): R_{T} = 3.40, m/z = 462.0 [M − H]⁻. |
| 213 (Example 180) | 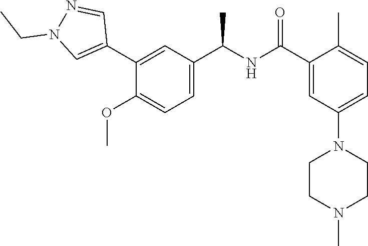 [SMILES: CCn1cc(-c2cc([C@@H](C)NC(=O)c3cc(N4CCN(C)CC4)ccc3C)ccc2OC)cn1] | N-[(1R)-1-[3-(1-Ethylpyrazol-4-yl)-4-methoxy-phenyl]ethyl]-2- methyl-5-(4-methylpiperazin-1-yl)benzamide ¹H NMR (500 MHz, DMSO-d₆) δ 8.57 (d, J = 8.5, 1H), 8.11 (s, 1H), 7.86 (s, 1H), 7.64 (d, J = 2.0, 1H), 7.20 (dd, J = 8.5, 2.0, 1H), 7.06 (d, J = 8.5, 1H), 7.01 (d, J = 8.5, 1H), 6.90 (dd, J = 8.5, 2.5, 1H), 6.83 (d, J = 2.5, 1H), 5.10 (quin, J = 7.0, 1H), 4.17 (q, J = 7.5, 2H), 3.86 (s, 3H), 3.11-3.09 (m, 4H), 2.45-2.43 (m, 4H), 2.22 (s, 3H), 2.17 (s, 3H), 1.43 (d, J = 7.0, 3H), 1.40 (t, J = 7.0, 3H). LC-MS (Method B): R_{T} = 3.49, m/z = 461.0 [M − H]⁻. |
| 214 (Example 180) | 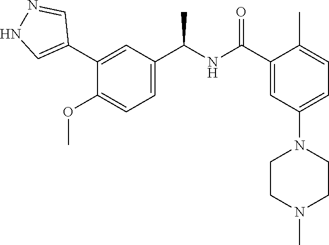 [SMILES: COc1ccc([C@@H](C)NC(=O)c2cc(N3CCN(C)CC3)ccc2C)cc1-c1cn[nH]c1] | N-[(1R)-1-[4-Methoxy-3-(1H-pyrazol-4-yl)phenyl]ethyl]-2- methyl-5-(4-methylpiperazin-1-yl)benzamide ¹H NMR (500 MHz, DMSO-d₆) δ 8.58 (d, J = 8.5, 1H), 8.01 (br s, 2H), 7.66 (d, J = 2.0, 1H), 7.20 (dd, J = 8.5, 2.5, 1H), 7.07 (d, J = 8.5, 1H), 7.01 (d, J = 8.5, 1H), 6.92 (dd, J = 8.5, 2.5, 1H), 6.85 (d, J = 2.5, 1H), 5.11 (quin, J = 7.0, 1H), 3.85 (s, 3H), 3.17-3.15 (m, 4H), 2.63-2.61 (m, 4H), 2.34 (br s, 3H), 2.18 (s, 3H), 1.44 (d, J = 7.0, 3H). LC-MS (Method B): R_{T} = 3.16, m/z = 432.9 [M − H]⁻. |
| 215 (Example 202) | 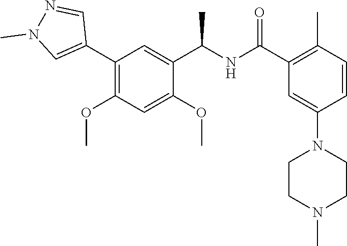 [SMILES: COc1cc(OC)c([C@@H](C)NC(=O)c2cc(N3CCN(C)CC3)ccc2C)cc1-c1cnn(C)c1] | N-[(1R)-1-[2,4-Dimethoxy-5-(1-methylpyrazol-4- yl)phenyl]ethyl]-2-methyl-5-(4-methylpiperazin-1-yl)benzamide ¹H NMR (500 MHz, CDCl₃) δ 7.82-7.79 (m, 1H), 7.75-7.72 (m, 1H), 7.38 (s, 1H), 7.09-7.06 (m, 1H), 6.95-6.92 (m, 1H), 6.89-6.85 (m, 1H), 6.64-6.58 (m, 1H), 6.55-6.52 (m, 1H), 5.45-5.37 (m, 1H), 3.93 (s, 3H), 3.91 (s, 3H), 3.89 (s, 3H), 3.20-3.13 (m, 4H), 2.59-2.53 (m, 4H), 2.35 (s, 3H), 2.31 (s, 3H), 1.55 (d, J = 7.0, 3H). LC-MS (Method A): R_{T} = 3.56, m/z = 478.8 [M + H]⁺. |
| 216 (Example 202) | 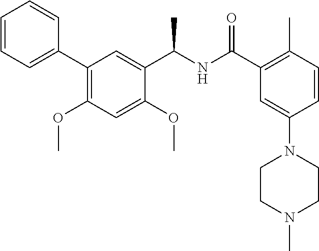 [SMILES: COc1cc(OC)c([C@@H](C)NC(=O)c2cc(N3CCN(C)CC3)ccc2C)cc1-c1ccccc1] | N-[(1R)-1-(2,4-Dimethoxy-5-phenyl-phenyl)ethyl]-2-methyl-5- (4-methylpiperazin-1-yl)benzamide ¹H NMR (500 MHz, CDCl₃) δ 7.48 (s, 2H), 7.38 (s, 2H), 7.29 (s, 1H), 7.26-7.25 (m, 1H), 7.24-7.22 (m, 1H), 7.08 (d, J = 8.4, 1H), 6.95-6.92 (m, 1H), 6.89-6.85 (m, 1H), 6.59-6.52 (m, 2H), 5.48-5.40 (m, 1H), 3.92 (s, 3H), 3.82 (s, 3H), 3.20-3.12 (m, 4H), 2.59-2.52 (m, 4H), 2.34 (s, 3H), 2.32 (s, 3H), 1.55 (d, J = 7.0, 3H). LC-MS (Method A): R_{T} = 4.27, m/z = 474.8 [M + H]⁺. |
| 217 (Example 187) | 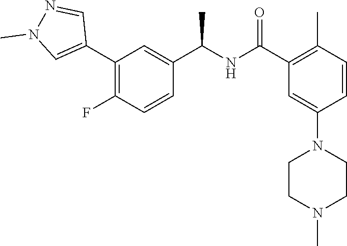 [SMILES: Cc1ccc(N2CCN(C)CC2)cc1C(=O)N[C@H](C)c1ccc(F)c(-c2cnn(C)c2)c1] | N-[(1R)-1-[4-Fluoro-3-(1-methylpyrazol-4-yl)phenyl]ethyl]-2- methyl-5-(4-methylpiperazin-1-yl)benzamide ¹H NMR (500 MHz, CDCl₃) δ 7.86-7.83 (m, 1H), 7.79-7.75 (m, 1H), 7.54 (dd, J = 2.1, 7.2, 1H), 7.21-7.16 (m, 1H), 7.12-7.06 (m, 2H), 6.92 (d, J = 2.6, 1H), 6.90-6.85 (m, 1H), 5.97-5.91 (m, 1H), 5.35-5.27 (m, 1H), 3.96 (s, 3H), 3.19-3.13 (m, 4H), 2.60-2.53 (m, 4H), 2.35 (s, 3H), 2.30 (s, 3H), 1.55 (d, J = 7.0, 3H). LC-MS (Method A): R_{T} = 3.46, m/z = 436.7 [M + H]⁺. |
| 218 (Example 187) | 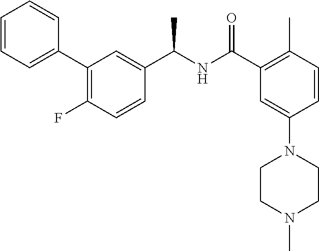 [SMILES: Cc1ccc(N2CCN(C)CC2)cc1C(=O)N[C@H](C)c1ccc(F)c(-c2ccccc2)c1] | N-[(1R)-1-(4-Fluoro-3-phenyl-phenyl)ethyl]-2-methyl-5-(4- methylpiperazin-1-yl)benzamide ¹H NMR (500 MHz, CDCl₃) δ 7.56-7.51 (m, 2H), 7.47-7.41 (m, 3H), 7.40-7.35 (m, 1H), 7.34-7.30 (m, 1H), 7.17-7.11 (m, 1H), 7.08 (d, J = 8.5, 1H), 6.92 (d, J = 2.6, 1H), 6.88 (dd, J = 2.6, 8.4, 1H), 5.98-5.90 (m, 1H), 5.39-5.31 (m, 1H), 3.19-3.12 (m, 4H), 2.60-2.53 (m, 4H), 2.35 (s, 3H), 2.31 (s, 3H), 1.55 (d, J = 7.0, 3H). LC-MS (Method A): R_{T} = 4.77, m/z = 432.7 [M + H]⁺. |
| 219 (Example 186) | 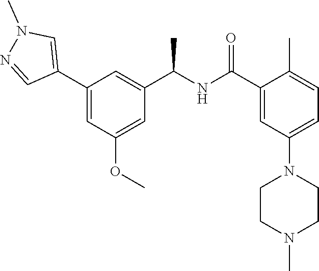 [SMILES: COc1cc(-c2cnn(C)c2)cc([C@@H](C)NC(=O)c2cc(N3CCN(C)CC3)ccc2C)c1] | N-[(1R)-1-[3-Methoxy-5-(1-methylpyrazol-4-yl)phenyl]ethyl]-2- methyl-5-(4-methylpiperazin-1-yl)benzamide ¹H NMR (500 MHz, CDCl₃) δ 7.73 (s, 1H), 7.60 (s, 1H), 7.10-7.05 (m, 2H), 6.94-6.90 (m, 2H), 6.90-6.85 (m, 1H), 6.78 (s, 1H), 6.00 (br d, J = 7.9, 1H), 5.30 (quin, J = 7.2, 1H), 3.94 (s, 3H), 3.84 (s, 3H), 3.21-3.11 (m, 4H), 2.59-2.53 (m, 4H), 2.34 (s, 3H), 2.32 (s, 3H), 1.60 (d, J = 7.0, 3H). LC-MS (Method B): R_{T} = 3.24 m/z = 446.9 [M − H]⁻. |
| 220 (Example 186) | 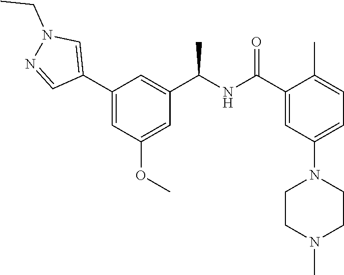 [SMILES: CCn1cc(-c2cc(OC)cc([C@@H](C)NC(=O)c3cc(N4CCN(C)CC4)ccc3C)c2)cn1] | N-[(1R)-1-[3-(1-Ethylpyrazol-4-yl)-5-methoxy-phenyl]ethyl]-2- methyl-5-(4-methylpiperazin-1-yl)benzamide ¹H NMR (500 MHz, CDCl₃) δ 7.78-7.73 (m, 1H), 7.64 (s, 1H), 7.11-7.06 (m, 2H), 6.94-6.91 (m, 2H), 6.87 (dd, J = 2.7, 8.5, 1H), 6.78 (s, 1H), 5.95 (br d, J = 7.9, 1H), 5.30 (quin, J = 7.1, 1H), 4.21 (q, J = 7.3, 2H), 3.79 (s, 3H), 3.21-3.12 (m, 4H), 2.62-2.50 (m, 4H), 2.34 (s, 3H), 2.33 (s, 3H), 1.60 (br d, J = 6.7, 3H), 1.53 (t, J = 7.3, 3H). LC-MS (Method B): R_{T} = 3.42, m/z = 461.0 [M − H]⁻. |
| 221 (Example 186) | 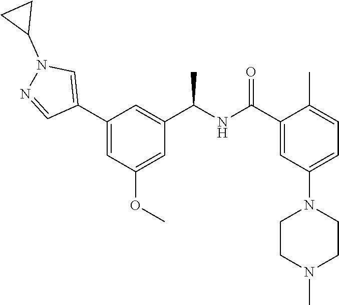 [SMILES: COc1cc(-c2cnn(C3CC3)c2)cc([C@@H](C)NC(=O)c2cc(N3CCN(C)CC3)ccc2C)c1] | N-[(1R)-1-[3-(1-Cyclopropylpyrazol-4-yl)-5-methoxy- phenyl]ethyl]-2-methyl-5-(4-methylpiperazin-1-yl)benzamide ¹H NMR (500 MHz, CDCl₃) δ 7.72 (s, 1H), 7.71-7.68 (m, 1H), 7.13-7.02 (m, 2H), 6.94-6.85 (m, 3H), 6.78 (s, 1H), 5.96 (br d, J = 7.9, 1H), 5.30 (quin, J = 7.2, 1H), 3.84 (s, 3H), 3.63 (tt, J = 3.8, 7.3, 1H), 3.23-3.12 (m, 4H), 2.66-2.53 (m, 4H), 2.36 (s, 3H), 2.33 (s, 3H), 1.60 (d, J = 6.7, 3H), 1.30-1.10 (m, 2H), 1.10-1.00 (m, 2H). LC-MS (Method B): R_{T} = 3.55; m/z = 474.8 [M − H]⁺. |
| 222 (Example 190) | 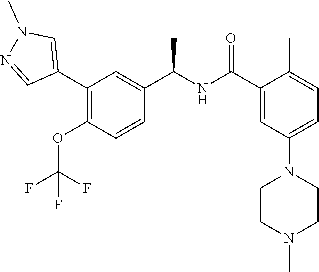 [SMILES: Cc1ccc(N2CCN(C)CC2)cc1C(=O)N[C@H](C)c1ccc(OC(F)(F)F)c(-c2cnn(C)c2)c1] | 2-Methyl-5-(4-methylpiperazin-1-yl)-N-[(1R)-1-[3-(1- methylpyrazol-4-yl)-4-(trifluoromethoxy)phenyl]ethyl]benzamide ¹H NMR (500 MHz, CDCl₃) δ 7.81 (s, 1H), 7.77-7.68 (m, 1H), 7.56 (d, J = 2.1, 1H), 7.32-7.21 (m, 2H), 7.09 (d, J = 8.2, 1H), 6.92 (d, J = 2.7, 1H), 6.91-6.86 (m, 1H), 5.97 (br d, J = 7.9, 1H), 5.38-5.29 (m, 1H), 3.96 (s, 3H), 3.22-3.10 (m, 4H), 2.62-2.51 (m, 4H), 2.34 (s, 3H), 2.32 (s, 3H), 1.61 (d, J = 7.0, 3H). LC-MS (Method B): R_{T} = 3.91, m/z = 501.0 [M − H]⁻. |
| 223 (Example 193) | 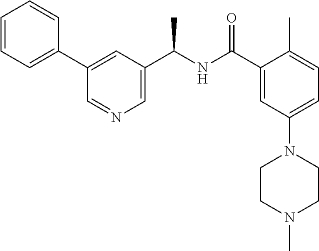 [SMILES: Cc1ccc(N2CCN(C)CC2)cc1C(=O)N[C@H](C)c1cncc(-c2ccccc2)c1] | 2-Methyl-5-(4-methylpiperazin-1-yl)-N-[(1R)-1-(5-phenyl-3- pyridyl)ethyl]benzamide ¹H NMR (500 MHz, DMSO-d₆) δ 8.79 (d, J = 2.0, 1H), 8.76 (d, J = 8.0, 1H), 8.62 (d, J = 1.5, 1H), 8.10 (br s, 1H), 7.73 (d, J = 7.5, 2H), 7.53 (t, J = 7.5, 2H), 7.45 (t, J = 7.5, 1H), 7.08 (d, J = 8.0, 1H), 6.93 (dd, J = 8.0, 2.5, 1H), 6.88 (d, J = 2.5, 1H), 5.24 (quin, J = 7.0, 1H), 3.14 (m, 4H), 2.56-2.54 (m, 4H), 2.30 (br s, 3H), 2.17 (s, 3H), 1.52 (d, J = 7.0, 3H). LC-MS (Method B): R_{T} = 3.46, m/z = 413.9 [M − H]⁻. |
| 224 (Example 193) | 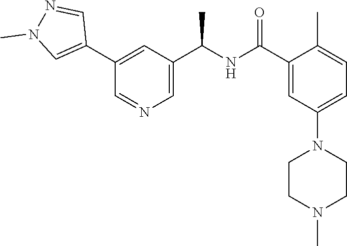 [SMILES: Cc1ccc(N2CCN(C)CC2)cc1C(=O)N[C@H](C)c1cncc(-c2cnn(C)c2)c1] | 2-Methyl-5-(4-methylpiperazin-1-yl)-N-[(1R)-1-[5-(1- methylpyrazol-4-yl)-3-pyridyl]ethyl]benzamide ¹H NMR (500 MHz, DMSO-d₆) δ 8.71-8.69 (m, 2H), 8.43 (d, J = 2.0, 1H), 8.23 (s, 1H), 7.96 (t, J = 2.0, 1H), 7.92 (s, 1H), 7.07 (d, J = 8.0, 1H), 6.91 (dd, J = 8.5, 2.5, 1H), 6.87 (d, J = 2.5, 1H), 5.16 (quin, J = 7.0, 1H), 3.89 (s, 3H), 3.12-3.10 (m, 4H), 2.46-2.44 (m, 4H), 2.22 (s, 3H), 2.16 (s, 3H), 1.49 (d, J = 7.0, 3H). LC-MS (Method B): R_{T} = 3.06, m/z = 417.9 [M − H]⁻. |
| 225 (Example 193) | 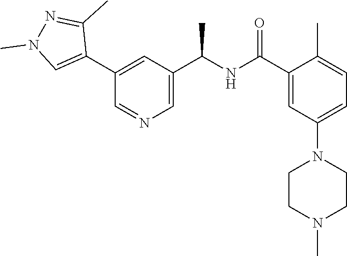 [SMILES: Cc1ccc(N2CCN(C)CC2)cc1C(=O)N[C@H](C)c1cncc(-c2cn(C)nc2C)c1] | N-[(1R)-1-[5-(1,3-Dimethylpyrazol-4-yl)-3-pyridyl]ethyl]-2- methyl-5-(4-methylpiperazin-1-yl)benzamide ¹H NMR (500 MHz, DMSO-d₆) δ 8.74 (d, J = 8.0, 1H), 8.54 (d, J = 2.0, 1H), 8.46 (d, J = 2.0, 1H), 7.99 (s, 1H), 7.83 (t, J = 2.0, 1H), 7.07 (d, J = 8.5, 1H), 6.92 (dd, J = 8.5, 2.5, 1H), 6.86 (d, J = 2.5, 1H), 5.18 (quin, J = 7.0, 1H), 3.81 (s, 3H), 3.12-3.10 (m, 4H), 2.50-2.48 (m, 4H), 2.31 (s, 3H), 2.25 (br s, 3H), 2.17 (s, 3H), 1.49 (d, J = 7.0, 3H). LC-MS (Method B): R_{T} = 2.95, m/z = 431.9 [M − H]⁻. |
| 226 (Example 194) | 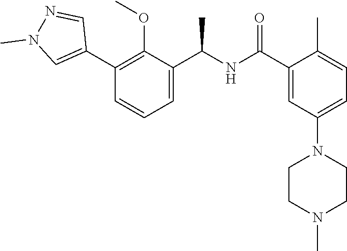 [SMILES: COc1c(-c2cnn(C)c2)cccc1[C@@H](C)NC(=O)c1cc(N2CCN(C)CC2)ccc1C] | N-[(1R)-1-[2-Methoxy-3-(1-methylpyrazol-4-yl)phenyl]ethyl]-2- methyl-5-(4-methylpiperazin-1-yl)benzamide ¹H NMR (500 MHz, DMSO-d₆) δ 8.68 (d, J = 8.0, 1H), 8.15 (s, 1H), 7.88 (s, 1H), 7.45 (dd, J = 7.5, 1.5, 1H), 7.33 (dd, J = 7.5, 1.5, 1H), 7.14 (t, J = 7.5, 1H), 7.06 (d, J = 8.0, 1H), 6.91 (dd, J = 8.0, 2.5, 1H), 6.85 (d, J = 2.5, 1H), 5.50 (quin, J = 7.0, 1H), 3.90 (s, 3H), 3.71 (s, 3H), 3.12-3.10 (m, 4H), 2.48-2.46 (m, 4H), 2.23 (s, 3H), 2.17 (s, 3H), 1.39 (d, J = 7.0, 3H). LC-MS (Method B): R_{T} = 3.39, m/z = 446.9 [M − H]⁻. |
| 227 (Example 181) | 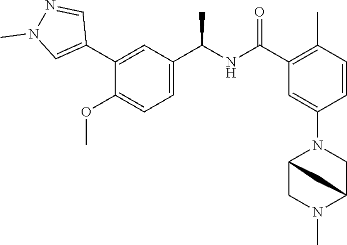 [SMILES: COc1ccc([C@@H](C)NC(=O)c2cc(N3C[C@H]4C[C@@H]3CN4C)ccc2C)cc1-c1cnn(C)c1] | N-[(1R)-1-[4-Methoxy-3-(1-methylpyrazol-4-yl)phenyl]ethyl]-2- methyl-5-[(1R,4R)-5-methyl-2,5-diazabicyclo[2.2.1]heptan-2- yl]benzamide ¹H NMR (500 MHz, DMSO-d₆) δ 8.54 (d, J = 8.5, 1H), 8.08 (s, 1H), 7.85 (s, 1H), 7.64 (d, J = 2.0, 1H), 7.20 (dd, J = 8.0, 2.0, 1H), 7.01 (d, J = 8.5, 1H), 6.99 (d, J = 8.5, 1H), 6.54 (dd, J = 8.0, 2.5, 1H), 6.47 (d, J = 2.5, 1H), 5.10 (quin, J = 7.0, 1H), 4.23 (br s, 1H), 3.88 (s, 3H), 3.86 (s, 3H), 3.38 (br s, 1H), 3.28 (dd, J = 9.0, 2.0, 1H), 3.14 (d, J = 9.0, 1H), 2.73 (dd, J = 9.5, 1.5, 1H), 2.44 (d, J = 9.5, 1H), 2.23 (s, 3H), 2.15 (s, 3H), 1.84 (br d, J = 9.0, 1H), 1.73 (br d, J = 9.0, 1H), 1.42 (d, J = 7.0, 3H). LC-MS (Method B): R_{T} = 3.82, m/z = 458.9 [M − H]⁻. |
| 228 (Example 196) | 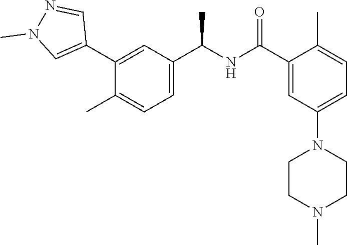 [SMILES: Cc1ccc(N2CCN(C)CC2)cc1C(=O)N[C@H](C)c1ccc(C)c(-c2cnn(C)c2)c1] | 2-Methyl-N-[(1R)-1-[4-methyl-3-(1-methylpyrazol-4- yl)phenyl]ethyl]-5-(4-methylpiperazin-1-yl)benzamide ¹H NMR (500 MHz, DMSO-d₆) δ 8.60 (d, J = 8.0, 1H), 7.92 (s, 1H), 7.64 (s, 1H), 7.40 (d, J = 1.5, 1H), 7.20 (d, J = 8.0, 1H), 7.16 (dd, J = 8.0, 1.5, 1H), 7.06 (d, J = 8.0, 1H), 6.90 (dd, J = 8.5, 2.5, 1H), 6.83 (d, J = 2.5, 1H), 5.09 (quin, J = 7.0, 1H), 3.89 (s, 3H), 3.09 (m, 4H), 2.45-2.43 (m, 4H), 2.34 (s, 3H), 2.22 (s, 3H), 2.17 (s, 3H), 1.42 (d, J = 7.0, 3H). LC-MS (Method B): R_{T} = 3.31, m/z = 430.9 [M − H]⁻. |
| 229 (Example 197) | 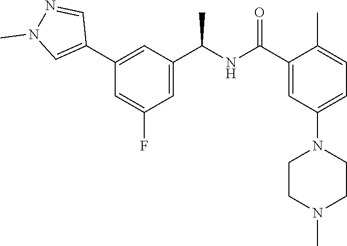 [SMILES: Cc1ccc(N2CCN(C)CC2)cc1C(=O)N[C@H](C)c1cc(F)cc(-c2cnn(C)c2)c1] | N-[(1R)-1-[3-Fluoro-5-(1-methylpyrazol-4-yl)phenyl]ethyl]-2- methyl-5-(4-methylpiperazin-1-yl)benzamide ¹H NMR (500 MHz, DMSO-d₆) δ 8.65 (d, J = 8.0, 1H), 8.18 (s, 1H), 7.89 (s, 1H), 7.46 (s, 1H), 7.29 (br d, J = 10.0, 1H), 7.07 (d, J = 8.5, 1H), 7.03 (br d, J = 10.0, 1H), 6.92 (dd, J = 8.5, 2.5, 1H), 6.87 (d, J = 2.5, 1H), 5.12 (quin, J = 7.0, 1H), 3.87 (s, 3H), 3.12-3.10 (m, 4H), 2.46-2.44 (m, 4H), 2.22 (s, 3H), 2.17 (s, 3H), 1.45 (d, J = 7.0, 3H). LC-MS (Method B): R_{T} = 3.25, m/z = 434.8 [M − H]⁻. |
| 230 (Example 198) | 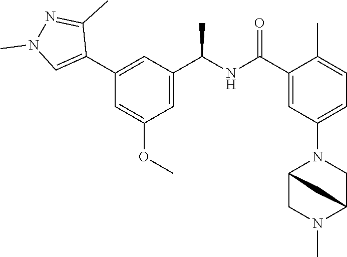 [SMILES: COc1cc(-c2cn(C)nc2C)cc([C@@H](C)NC(=O)c2cc(N3C[C@H]4C[C@@H]3CN4C)ccc2C)c1] | N-[(1R)-1-[3-(1,3-Dimethylpyrazol-4-yl)-5-methoxy- phenyl]ethyl]-2-methyl-5-[(1R,4R)-5-methyl-2,5- diazabicyclo[2.2.1]heptan-2-yl]benzamide ¹H NMR (500 MHz, DMSO-d₆) δ 8.61 (d, J = 8.0, 1H), 7.86 (s, 1H), 7.04 (br s, 1H), 6.99 (d, J = 8.5, 1H), 6.85 (br s, 1H), 6.81 (br s, 1H), 6.55 (dd, J = 8.5, 2.5, 1H), 6.48 (d, J = 2.5, 1H), 5.10 (quin, J = 7.0, 1H), 4.22 (br s, 1H), 3.78 (s, 6H), 3.39 (br s, 1H), 3.28 (dd, J = 9.0, 1.5, 1H), 3.14 (d, J = 9.0, 1H), 2.73 (m, 1H), 2.45 (d, J = 9.0, 1H), 2.30 (s, 3H), 2.23 (s, 3H), 2.16 (s, 3H), 1.84 (br d, J = 9.0, 1H), 1.73 (br d, J = 9.0, 1H), 1.43 (d, J = 7.0, 3H). LC-MS (Method B): R_{T} = 3.74, m/z = 472.9 [M − H]⁻. |
| 231 | 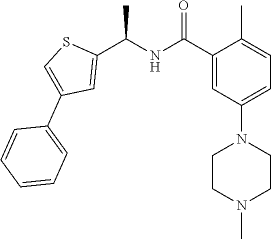 [SMILES: Cc1ccc(N2CCN(C)CC2)cc1C(=O)N[C@H](C)c1cc(-c2ccccc2)cs1] | 2-Methyl-5-(4-methylpiperazin-1-yl)-N-[(1R)-1-(4-phenyl-2- thienyl)ethyl]benzamide ¹H NMR (500 MHz, DMSO-d₆) δ 8.77 (d, J = 8.0, 1H), 7.70 (d, J = 1.5, 1H), 7.68-7.67 (m, 2H), 7.43-7.39 (m, 3H), 7.29-7.27 (m, 1H), 7.07 (d, J = 8.0, 1H), 6.91 (dd, J = 8.0, 3.0, 1H), 6.88 (d, J = 3.0, 1H), 5.39 (quin, J = 7.0, 1H), 3.11-3.09 (m, 4H), 2.45-2.43 (m, 4H), 2.24 (s, 3H), 2.21 (s, 3H), 1.58 (d, J = 7.0, 3H). LC-MS (Method B): R_{T} = 4.00, m/z = 418.8 [M − H]⁻. |
| 232 | 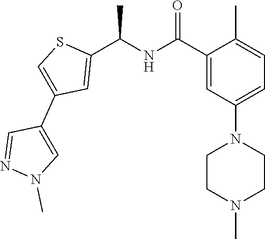 [SMILES: Cc1ccc(N2CCN(C)CC2)cc1C(=O)N[C@H](C)c1cc(-c2cnn(C)c2)cs1] | 2-Methyl-5-(4-methylpiperazin-1-yl)-N-[(1R)-1-[4-(1- methylpyrazol-4-yl)-2-thienyl]ethyl]benzamide ¹H NMR (500 MHz, DMSO-d₆) δ 8.74 (d, J = 8.5, 1H), 7.99 (s, 1H), 7.72 (s, 1H), 7.36 (m, 1H), 7.21 (s, 1H), 7.07 (d, J = 8.5, 1H), 6.91 (dd, J = 8.5, 2.5, 1H), 6.87 (d, J = 2.5, 1H), 5.36 (quin, J = 7.0, 1H), 3.84 (s, 3H), 3.11 (m, 4H), 2.44 (m, 4H), 2.23 (s, 3H), 2.22 (s, 3H), 1.56 (d, J = 7.0, 3H). LC-MS (Method B): R_{T} = 3.27, m/z = 422.8 [M − H]⁻. |
| 233 (Example 186) | 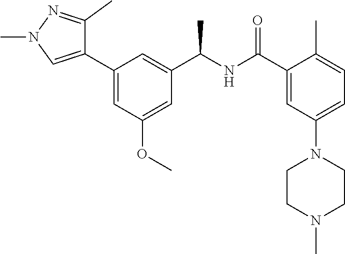 [SMILES: COc1cc(-c2cn(C)nc2C)cc([C@@H](C)NC(=O)c2cc(N3CCN(C)CC3)ccc2C)c1] | N-[(1R)-1-[3-(1,3-Dimethylpyrazol-4-yl)-5-methoxy- phenyl]ethyl]-2-methyl-5-(4-methylpiperazin-1-yl)benzamide ¹H NMR (500 MHz, CDCl₃) δ 7.43 (s, 1H), 7.08 (d, J = 8.5, 1H), 6.98 (s, 1H), 6.93 (d, J = 2.7, 1H), 6.89-6.86 (m, 1H), 6.86-6.79 (m, 2H), 5.94 (br d, J = 7.9, 1H), 5.31 (quin, J = 7.2, 1H), 3.87 (s, 3H), 3.84 (s, 3H), 3.19-3.12 (m, 4H), 2.61-2.50 (m, 4H), 2.40 (s, 3H), 2.34 (s, 3H), 2.33 (s, 3H), 1.65-1.54 (m, 3H). LC-MS (Method B): R_{T} = 3.35, m/z = 460.9 [M − H]⁻. |
| 234 (Example 186) | 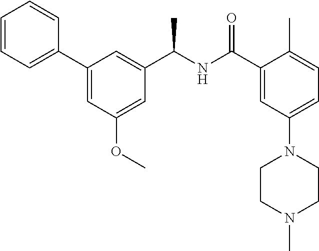 [SMILES: COc1cc(-c2ccccc2)cc([C@@H](C)NC(=O)c2cc(N3CCN(C)CC3)ccc2C)c1] | N-[(1R)-1-(3-Methoxy-5-phenyl-phenyl)ethyl]-2-methyl-5-(4- methylpiperazin-1-yl)benzamide ¹H NMR (500 MHz, CDCl₃) δ = 7.60-7.54 (m, 2H), 7.46-7.40 (m, 2H), 7.38-7.33 (m, 1H), 7.19-7.17 (m, 1H), 7.11-7.06 (m, 1H), 7.05-7.01 (m, 1H), 6.95-6.90 (m, 2H), 6.90-6.85 (m, 1H), 6.01-5.94 (m, 1H), 5.40-5.31 (m, 1H), 3.87 (s, 3H), 3.19-3.11 (m, 4H), 2.58-2.52 (m, 4H), 2.36-2.32 (m, 6H), 1.62 (d, J = 7.0, 3H). LC-MS (Method A): R_{T} = 4.15, m/z = 444.8 [M + H]⁺. |

### Example 235: N-[(1R)-1-[3-Methoxy-5-(1-methylpyrazol-4-yl)phenyl]ethyl]-2-methyl-5-piperazin-1-yl-benzamide

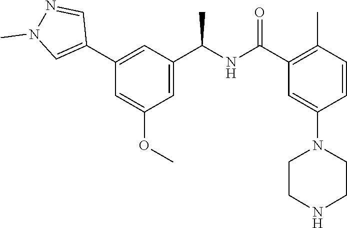 [SMILES: COc1cc(-c2cnn(C)c2)cc([C@@H](C)NC(=O)c2cc(N3CCNCC3)ccc2C)c1]

Using General Procedure 4 with tert-butyl 4-[3-[[(1R)-1-[3-methoxy-5-(1-methylpyrazol-4-yl)phenyl]ethyl]carbamoyl]-4-methyl-phenyl]piperazine-1-carboxylate (220 mg, 412 μmol)—prepared in a similar manner to N-[(1R)-1-(4-methoxy-3-phenyl-phenyl)ethyl]-2-methyl-5-(4-methylpiperazin-1-yl)benzamide (Example 203)—gave N-[(1R)-1-[3-methoxy-5-(1-methylpyrazol-4-yl)phenyl]ethyl]-2-methyl-5-piperazin-1-yl-benzamide (152 mg, 81%) as a pale yellow solid. ¹H NMR (500 MHz, DMSO-d₆) δ 8.61 (d, J=8.0, 1H), 8.12 (s, 1H), 7.83 (s, 1H), 7.19 (br s, 1H), 7.06 (d, J=8.5, 1H), 6.99-6.97 (m, 1H), 6.89 (dd, J=8.5, 2.5, 1H), 6.84 (d, J=2.5, 1H), 6.83-6.81 (m, 1H), 5.09 (quin, J=7.0, 1H), 3.87 (s, 3H), 3.79 (s, 3H), 3.02-3.00 (m, 4H), 2.84-2.82 (m, 4H), 2.19 (s, 3H), 1.43 (d, J=7.0, 3H). LC-MS (Method B): R_{T}=3.33, m/z=432.8 [M−H]⁻.

### Example 236: N-[(1R)-1-[4-Methoxy-3-(5-methyl-1,3,4-thiadiazol-2-yl)phenyl]ethyl]-2-methyl-5-(4-methylpiperazin-1-yl)benzamide

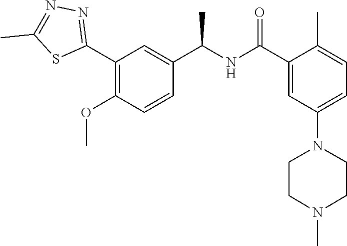 [SMILES: COc1ccc([C@@H](C)NC(=O)c2cc(N3CCN(C)CC3)ccc2C)cc1-c1nnc(C)s1]

### Step A: N-[(1R)-1-[4-Methoxy-3-(4,4,5,5-tetramethyl-1,3,2-dioxaborolan-2-yl)phenyl]ethyl]-2-methyl-5-(4-methylpiperazin-1-yl)benzamide

N-[(1R)-1-(3-Bromo-4-methoxy-phenyl)ethyl]-2-methyl-5-(4-methylpiperazin-1-yl)benzamide (1.00 g, 2.24 mmol) (Example 180), bis(pinacolato)diboron (853 mg, 3.36 mmol) potassium acetate (659 mg, 6.72 mmol) and 1,1′-bis(diphenylphosphino)ferrocenepalladium (II) dichloride (163 mg, 224 μmol) were added to 1,4-dioxane (40 mL) and heated at 100° C. under nitrogen for 2 hours. The reaction was concentrated, diluted with diethyl ether (100 ml), and filtered to afford a dark gum. Purification by FCC (eluting with ethyl acetate and then 20-30% MeOH in ethyl acetate) afforded N-[(1R)-1-[4-methoxy-3-(4,4,5,5-tetramethyl-1,3,2-dioxaborolan-2-yl)phenyl]ethyl]-2-methyl-5-(4-methylpiperazin-1-yl)benzamide (730 mg, 33%) as a yellow foam. LC-MS (Method B): R_{T}=3.75, m/z=494.9 [M−H]⁻.

### Step B: N-[(1R)-1-[4-methoxy-3-(5-methyl-1,3,4-thiadiazol-2-yl)phenyl]ethyl]-2-methyl-5-(4-methylpiperazin-1-yl)benzamide

Using General Procedure 2 with 2-bromo-5-methyl-1,3,4-thiadiazole (108 mg, 0.61 mmol) and N-[(1R)-1-[4-methoxy-3-(4,4,5,5-tetramethyl-1,3,2-dioxaborolan-2-yl)phenyl]ethyl]-2-methyl-5-(4-methylpiperazin-1-yl)benzamide (200 mg, 0.40 mmol) at 80° C. for 4 hours afforded N-[(1R)-1-[4-methoxy-3-(5-methyl-1,3,4-thiadiazol-2-yl)phenyl]ethyl]-2-methyl-5-(4-methylpiperazin-1-yl)benzamide (25 mg, 13%) as a white foam. ¹H NMR (500 MHz, CDCl₃) δ 8.47 (d, J=2.4, 1H), 7.59-7.43 (m, 1H), 7.07 (d, J=8.5, 1H), 7.04-7.00 (m, 1H), 6.94 (d, J=2.4, 1H), 6.92-6.81 (m, 1H), 6.04 (br d, J=7.9, 1H), 5.33 (quin, J=7.1, 1H), 4.00 (s, 3H), 3.22-3.14 (m, 4H), 2.81 (s, 3H), 2.62-2.55 (m, 4H), 2.35 (s, 3H), 2.32-2.31 (m, 3H), 1.63 (d, J=7.0, 3H). LC-MS (Method B): R_{T}=3.40, m/z=464.9 [M−H]⁻.

### Example 237: N-[(1R)-1-[4-Methoxy-3-(4-piperidyl)phenyl]ethyl]-2-methyl-5-(4-methylpiperazin-1-yl)benzamide

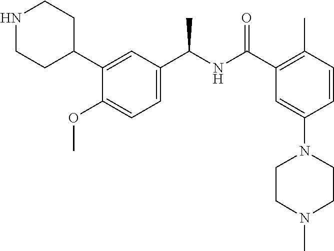 [SMILES: COc1ccc([C@@H](C)NC(=O)c2cc(N3CCN(C)CC3)ccc2C)cc1C1CCNCC1]

### Step A: tert-Butyl 4-[2-methoxy-5-[(1R)-1-[[2-methyl-5-(4-methylpiperazin-1-yl)benzoyl]amino]ethyl]phenyl]piperidine-1-carboxylate

Using General Procedure 5 with tert-butyl 4-[2-methoxy-5-[(1R)-1-[[2-methyl-5-(4-methylpiperazin-1-yl)benzoyl]amino]ethyl]phenyl]-3,6-dihydro-2H-pyridine-1-carboxylate (235 mg, 0.43 mmol)—prepared in a similar manner to N-[(1R)-1-(4-Methoxy-3-phenyl-phenyl)ethyl]-2-methyl-5-(4-methylpiperazin-1-yl)benzamide (Example 203)—and palladium hydroxide, 20% on carbon (18 mg, 128 μmol) gave tert-butyl 4-[2-methoxy-5-[(1R)-1-[[2-methyl-5-(4-methylpiperazin-1-yl)benzoyl]amino]ethyl]phenyl]piperidine-1-carboxylate (234 mg, 99%) as a white foam. LC-MS (Method B): R_{T}=4.13, m/z=550.1 [M−H]⁻.

### Step B: N-[(1R)-1-[4-methoxy-3-(4-piperidyl)phenyl]ethyl]-2-methyl-5-(4-methylpiperazin-1-yl)benzamide

Using General Procedure 4 with tert-butyl 4-[2-methoxy-5-[(1R)-1-[[2-methyl-5-(4-methylpiperazin-1-yl)benzoyl]amino]ethyl]phenyl]piperidine-1-carboxylate (230 mg, 0.41 mmol) gave N-[(1R)-1-[4-methoxy-3-(4-piperidyl)phenyl]ethyl]-2-methyl-5-(4-methylpiperazin-1-yl)benzamide (161 mg, 77%) as a white foam. ¹H NMR (500 MHz, CDCl₃) δ 7.24-7.15 (m, 2H), 7.09-7.05 (m, 1H), 6.93-6.90 (m, 1H), 6.89-6.80 (m, 2H), 5.85 (br s, 1H), 5.33-5.21 (m, 1H), 3.82 (s, 3H), 3.20-3.12 (m, 6H), 3.19-3.05 (m, 1H), 2.77 (dt, J=2.3, 12.1, 2H), 2.59-2.52 (m, 4H), 2.34 (s, 3H), 2.31 (s, 3H), 1.82-1.71 (m, 2H), 1.64-1.50 (m, 5H). LC-MS (Method B): R_{T}=3.65, m/z=450.0 [M−H]⁻.

### Example 238: 3-[4-[2-Methoxy-5-[(1R)-1-[[2-methyl-5-(4-methylpiperazin-1-yl)benzoyl]amino]ethyl]phenyl]-1-piperidyl]propanoic Acid

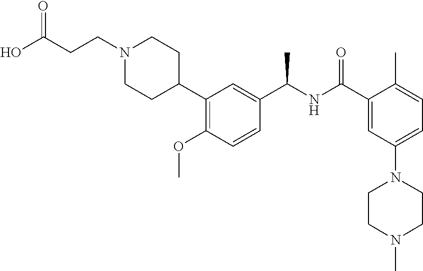 [SMILES: COc1ccc([C@@H](C)NC(=O)c2cc(N3CCN(C)CC3)ccc2C)cc1C1CCN(CCC(=O)O)CC1]

### Step A: Methyl 3-[4-[2-methoxy-5-[(1R)-1-[[2-methyl-5-(4-methylpiperazin-1-yl)benzoyl]amino]ethyl]phenyl]-1-piperidyl]propanoate

N-[(1R)-1-[4-Methoxy-3-(4-piperidyl)phenyl]ethyl]-2-methyl-5-(4-methylpiperazin-1-yl)benzamide (82 mg, 0.18 mmol) (Example 237) was dissolved in MeOH (10 mL). To this was added benzyl acrylate (44 mg, 0.27 mmol) and the mixture was stirred for 2 days. The mixture was evaporated and purified by FCC (eluting with ethyl acetate, then 20% ethyl acetate in MeOH, then 20% 7.0 N NH₃ in MeOH in ethyl acetate) to afford methyl 3-[4-[2-methoxy-5-[(1R)-1-[[2-methyl-5-(4-methylpiperazin-1-yl)benzoyl]amino]ethyl]phenyl]-1-piperidyl]propanoate (86 mg, 88%) as a clear gum. LC-MS (Method B): R_{T}=3.57, m/z=536.1 [M−H]⁻.

### Step B: 3-[4-[2-Methoxy-5-[(1R)-1-[[2-methyl-5-(4-methylpiperazin-1-yl)benzoyl]amino]ethyl]phenyl]-1-piperidyl]propanoic Acid

Methyl 3-[4-[2-methoxy-5-[(1R)-1-[[2-methyl-5-(4-methylpiperazin-1-yl)benzoyl]amino]ethyl] phenyl]-1-piperidyl]propanoate (73 mg, 0.13 mmol) was added to MeOH (5 mL) and water (5 mL). To this was added lithium hydroxide monohydrate (6.33 mg, 150 μmol) and the mixture was stirred over the weekend. The mixture was evaporated and then passed down a SCX column eluting with MeOH and then 3.5 NH₃ in MeOH to afford a yellow film. This was dissolved in DCM (1 mL), to this was added 60% diethyl ether in petroleum ether to afford a solid which was sonicated and filtered to afford 3-[4-[2-methoxy-5-[(1R)-1-[[2-methyl-5-(4-methylpiperazin-1-yl)benzoyl]amino]ethyl]phenyl]-1-piperidyl]propanoic acid (39 mg, 53%) as a beige solid. ¹H NMR (500 MHz, CDCl₃) δ 7.24-7.19 (m, 1H), 7.19-7.16 (m, 1H), 7.08 (d, J=8.2, 1H), 6.93-6.86 (m, 2H), 6.86-6.82 (m, 1H), 6.00 (br d, J=7.9, 1H), 5.25 (quin, J=7.1, 1H), 3.82 (s, 3H), 3.28 (br d, J=11.6, 2H), 3.22-3.13 (m, 4H), 3.13-2.95 (m, 1H), 2.93-2.77 (m, 2H), 2.65-2.58 (m, 4H), 2.58-2.39 (m, 4H), 2.36-2.34 (m, 3H), 2.32 (s, 3H), 1.96-1.81 (m, 4H), 1.56 (d, J=6.7, 3H). LC-MS (Method B): R_{T}=2.45, m/z=522.1 [M−H]⁻.

### Example 239: N-[(1R)-1-[2-Methoxy-5-(1-methylpyrazol-4-yl)phenyl]ethyl]-2-methyl-5-(4-methylpiperazin-1-yl)benzamide

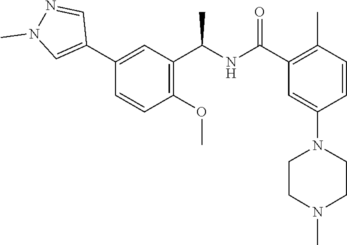 [SMILES: COc1ccc(-c2cnn(C)c2)cc1[C@@H](C)NC(=O)c1cc(N2CCN(C)CC2)ccc1C]

### Step A: (NE,S)—N-[(5-Bromo-2-methoxy-phenyl)methylene]-2-methyl-propane-2-sulfinamide

(S)-(−)-2-Methylpropane-2-sulphonamide (2.80 g, 23.1 mmol) was added to a solution of 5-bromo-2-methoxy-benzaldehyde (4.96 g, 23.1 mmol) and cesium carbonate (7.52 g, 23.1 mmol) in DCM (30 mL) and the reaction mixture heated to reflux over the weekend. The reaction mixture was allowed to cool to RT. Water (120 mL) and DCM (120 mL) were added and the phases separated. The aqueous phase was extracted with DCM (70 mL). The combined organic phases were washed with brine (150 mL), dried over sodium sulfate and the solvent removed in vacuo which gave (NE,S)—N-[(5-bromo-2-methoxy-phenyl)methylene]-2-methyl-propane-2-sulfinamide (6.68. g, 91%) as a yellow oil. ¹H NMR (500 MHz, CDCl₃) δ 8.97 (s, 1H), 8.07 (d, J=2.5, 1H), 7.55 (dd, J=9.0, 2.5, 1H), 6.86 (d, J=9.0, 1H), 3.88 (s, 3H), 1.29 (s, 9H).

### Step B: (S)—N-[(1R)-1-(5-Bromo-2-methoxy-phenyl)ethyl]-2-methyl-propane-2-sulfinamide

(NE,S)—N-[(5-Bromo-2-methoxy-phenyl)methylene]-2-methyl-propane-2-sulfinamide (6.68 g, 21.0 mmol) was dissolved in THF (110 mL) and cooled to −30° C. under nitrogen atmosphere. Once cooled, methylmagnesium bromide solution (3.0 M, 8.40 mL) was slowly added to afford a yellow solution. The mixture was stirred at −30° C. for 1 hour before being allowed to warm up to RT overnight. Water (30 mL), 2N HCl (5 mL) and diethyl ether (70 mL) were added, and the phases separated. The organic phase was washed with brine (100 mL), dried over sodium sulfate and the solvent removed in vacuo. Purification by FCC (eluting with 0-50% ethyl acetate in diethyl ether followed by 0-10% MeOH in ethyl acetate) gave (S)—N-[(1R)-1-(5-bromo-2-methoxy-phenyl)ethyl]-2-methyl-propane-2-sulfinamide (1.80 g, 26%) as a pale yellow oil. ¹H NMR (500 MHz, CDCl₃) δ 7.40 (d, J=2.5, 1H), 7.32 (dd, J=9.0, 2.5, 1H), 6.75 (d, J=9.0, 1H), 4.86-4.84 (m, 1H), 3.82 (s, 3H), 3.48-3.46 (m, 1H), 1.50 (d, J=7.0, 3H), 1.21 (s, 9H).

### Step C: (S)—N-[(1R)-1-[2-methoxy-5-(1-methylpyrazol-4-yl)phenyl]ethyl]-2-methyl-propane-2-sulfinamide

Using General Procedure 2 with (S)—N-[(1R)-1-(5-bromo-2-methoxy-phenyl)ethyl]-2-methyl-propane-2-sulfinamide (1.02 g, 3.05 mmol) and 1-methyl-4-(4,4,5,5-tetramethyl-1,3,2-dioxaborolan-2-yl)pyrazole (698 mg, 3.36 mmol) at 85° C. for 3 hours gave (S)—N-[(1R)-1-[2-methoxy-5-(1-methylpyrazol-4-yl)phenyl]ethyl]-2-methyl-propane-2-sulfinamide (1.00 g, 98%) as yellow oil. LC-MS (Method B): R_{T}=3.14, m/z=334.7 [M−H]⁻.

### Step D: (1R)-1-[2-Methoxy-5-(1-methylpyrazol-4-yl)phenyl]ethanamine Hydrochloride Salt

Using General Procedure 4 with (S)—N-[(1R)-1-[2-methoxy-5-(1-methylpyrazol-4-yl)phenyl]ethyl]-2-methyl-propane-2-sulfinamide (1.02 g, 3.04 mmol) gave (1R)-1-[2-methoxy-5-(1-methylpyrazol-4-yl)phenyl]ethanamine hydrochloride salt (798 mg, 98%) as a white solid. ¹H NMR (500 MHz, DMSO-d₆) δ 8.45 (br s, 3H), 8.06 (s, 1H), 7.82 (s, 1H), 7.80 (d, J=2.0, 1H), 7.53 (dd, J=8.5, 2.5, 1H), 7.07 (d, J=8.5, 1H), 4.59-4.57 (m, 1H), 3.86 (s, 3H), 3.85 (s, 3H), 1.51 (d, J=7.0, 3H).

### Step E: N-[(1R)-1-[2-Methoxy-5-(1-methylpyrazol-4-yl)phenyl]ethyl]-2-methyl-5-(4-methylpiperazin-1-yl)benzamide

Using General Procedure 1 with (1R)-1-[2-methoxy-5-(1-methylpyrazol-4-yl)phenyl]ethanamine hydrochloride salt (86 mg, 321 μmol) and 2-methyl-5-(4-methylpiperazin-1-yl)benzoic acid (83 mg, 353 μmol) (Example 21, Step B) with purification by FCC (eluting with 0-100% MeOH in DCM) gave N-[(1R)-1-[2-methoxy-5-(1-methylpyrazol-4-yl)phenyl]ethyl]-2-methyl-5-(4-methylpiperazin-1-yl)benzamide (78 mg, 52%) as a white crystalline solid. ¹H NMR (500 MHz, DMSO-d₆) δ 8.56 (d, J=8.5, 1H), 7.93 (s, 1H), 7.68 (s, 1H), 7.60 (d, J=2.0, 1H), 7.39 (dd, J=8.5, 2.0, 1H), 7.07 (d, J=8.0, 1H), 6.98 (d, J=8.0, 1H), 6.92 (dd, J=8.0, 2.5, 1H), 6.86 (d, J=2.5, 1H), 5.43 (quin, J=7.0, 1H), 3.86 (s, 3H), 3.85 (s, 3H), 3.12-3.10 (m, 4H), 2.46-2.44 (m, 4H), 2.23 (s, 3H), 2.18 (s, 3H), 1.36 (d, J=7.0, 3H). LC-MS (Method B): R_{T}=3.29, m/z=446.9 [M−H]⁻.

### Further Examples

The following examples were prepared in a similar manner to N-[(1R)-1-[2-methoxy-5-(1-methylpyrazol-4-yl)phenyl]ethyl]-2-methyl-5-(4-methylpiperazin-1-yl)benzamide (Example 239), using the required commercially available aldehyde in Step A, the required commercially available boronic acid or ester in Step D, and the required carboxylic acid—prepared in a similar manner to 2-methyl-5-(4-methylpiperazin-1-yl)benzoic acid hydrochloride salt (Example 21, Step B)—in Step E. Examples which did not provide solid material after Step E were treated with 2N HCl in diethyl ether, then concentrated and triturated with an appropriate solvent to give product as a hydrochloride salt.

| Example | Structure | -Name and Analytical Data |
|---|---|---|
| 240 | 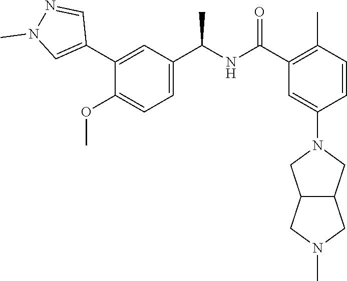 [SMILES: COc1ccc([C@@H](C)NC(=O)c2cc(N3CC4CN(C)CC4C3)ccc2C)cc1-c1cnn(C)c1] | N-[(1R)-1-[4-Methoxy-3-(1-methylpyrazol-4-yl)phenyl]ethyl]-2- methyl-5-(2-methyl-1,3,3a,4,6,6a-hexahydropyrrolo[3,4-c]pyrrol-5- yl)benzamide ¹H NMR (500 MHz, DMSO-d₆) δ 8.54 (d, J = 8.5, 1H), 8.08 (s, 1H), 7.85 (s, 1H), 7.64 (d, J = 2.0, 1H), 7.20 (dd, J = 8.5, 2.0, 1H), 7.01 (d, J = 8.0, 2H), 6.61 (dd, J = 8.5, 2.5, 1H), 6.57 (d, J = 2.5, 1H), 5.10 (quin, J = 7.0, 1H), 3.88 (s, 3H), 3.86 (s, 3H), 3.32-3.30 (m, 2H), 3.05-3.03 (m, 2H), 2.87-2.85 (m, 2H), 2.54-2.52 (m, 2H), 2.40-2.38 (m, 2H), 2.22 (s, 3H), 2.16 (s, 3H), 1.43 (d, J = 7.0, 3H). LC-MS (Method B): R_{T} = 3.78, m/z = 472.9 [M − H]⁻. |
| 241 | 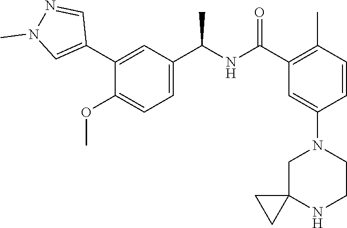 [SMILES: COc1ccc([C@@H](C)NC(=O)c2cc(N3CCNC4(CC4)C3)ccc2C)cc1-c1cnn(C)c1] | 5-(4,7-Diazaspiro[2.5]octan-7-yl)-N-[(1R)-1-[4-methoxy-3-(1- methylpyrazol-4-yl)phenyl]ethyl]-2-methyl-benzamide ¹H NMR (500 MHz, DMSO-d₆) δ 8.56 (d, J = 8.0, 1H), 8.06 (s, 1H), 7.84 (s, 1H), 7.63 (d, J = 2.0, 1H), 7.19 (dd, J = 8.0, 2.0, 1H), 7.04 (d, J = 8.5, 1H), 7.01 (d, J = 8.5, 1H), 6.85 (dd, J = 8.0, 2.5, 1H), 6.78 (d, J = 2.5, 1H), 5.10 (quin, J = 7.0, 1H), 3.88 (s, 3H), 3.86 (s, 3H), 3.04-3.02 (m, 2H), 2.90-2.88 (m, 4H), 2.17 (s, 3H), 1.42 (d, J = 7.0, 3H), 0.51-0.49 (m, 4H). LC-MS (Method B): R_{T} = 3.22, m/z = 458.9 [M − H]⁻. |
| 242 | 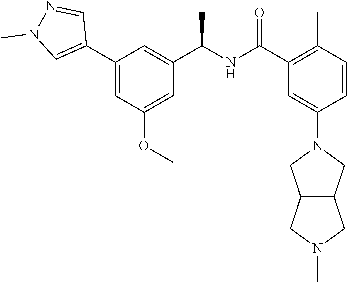 [SMILES: COc1cc(-c2cnn(C)c2)cc([C@@H](C)NC(=O)c2cc(N3CC4CN(C)CC4C3)ccc2C)c1] | N-[(1R)-1-[3-Methoxy-5-(1-methylpyrazol-4-yl)phenyl]ethyl]-2- methyl-5-(2-methyl-1,3,3a,4,6,6a-hexahydropyrrolo[3,4-c]pyrrol-5- yl)benzamide ¹H NMR (500 MHz, DMSO-d₆) δ 8.58 (d, J = 8.5, 1H), 8.12 (s, 1H), 7.83 (s, 1H), 7.19 (br s, 1H), 7.02 (d, J = 8.0, 1H), 6.99 (m, 1H), 6.82 (br s, 1H), 6.62 (dd, J = 8.0, 2.5, 1H), 6.59 (d, J = 2.5, 1H), 5.09 (quin, J = 7.0, 1H), 3.87 (s, 3H), 3.79 (s, 3H), 3.06-3.04 (m, 2H), 2.88-2.86 (m, 2H), 2.58-2.56 (m, 2H), 2.40-2.39 (m, 2H), 2.23 (s, 3H), 2.17 (s, 3H), 1.43 (d, J = 7.0, 3H). LC-MS (Method B): R_{T} = 3.82, m/z = 472.9 [M − H]⁻. |
| 243 | 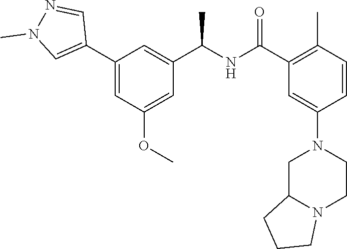 [SMILES: COc1cc(-c2cnn(C)c2)cc([C@@H](C)NC(=O)c2cc(N3CCN4CCCC4C3)ccc2C)c1] | 5-(3,4,6,7,8,8a-Hexahydro-1H-pyrrolo[1,2-a]pyrazin-2-yl)-N-[(1R)-1- [3-methoxy-5-(1-methylpyrazol-4-yl)phenyl]ethyl]-2-methyl- benzamide ¹H NMR (500 MHz, DMSO-d₆) δ 8.61 (d, J = 8.5, 1H), 8.12 (s, 1H), 7.83 (s, 1H), 7.19 (s, 1H), 7.06 (d, J = 8.0, 1H), 6.99 (br s, 1H), 6.93 (dd, J = 8.5, 2.5, 1H), 6.87 (br s, 1H), 6.82 (br s, 1H), 5.09 (quin, J = 7.0, 1H), 3.87 (s, 3H), 3.79 (s, 3H), 3.74 (br d, J = 11.0, 1H), 3.58 (br d, J = 11.0, 1H), 3.05-3.00 (m, 2H), 2.71-2.70 (m, 1H), 2.37 (td, J = 10.5, 5.0, 1H), 2.22-2.21 (m, 1H), 2.18 (s, 3H), 2.04-2.03 (m, 2H), 1.81 (m, 1H), 1.75-1.66 (m, 2H), 1.44 (d, J = 7.0, 3H), 1.36-1.35 (m, 1H). LC-MS (Method B): R_{T} = 3.47, m/z = 472.9 [M − H]⁻. |
| 244 | 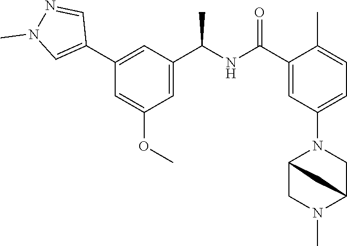 [SMILES: COc1cc(-c2cnn(C)c2)cc([C@@H](C)NC(=O)c2cc(N3C[C@H]4C[C@@H]3CN4C)ccc2C)c1] | N-[(1R)-1-[3-Methoxy-5-(1-methylpyrazol-4-yl)phenyl]ethyl]-2- methyl-5-[(1R,4R)-5-methyl-2,5-diazabicyclo[2.2.1]heptan-2- yl]benzamide ¹H NMR (500 MHz, DMSO-d₆) δ 8.58 (d, J = 8.0, 1H), 8.12 (s, 1H), 7.83 (s, 1H), 7.19 (s, 1H), 7.02 (m, 2H), 6.82 (br s, 1H), 6.55 (dd, J = 8.0, 2.5, 1H), 6.49 (d, J = 2.5, 1H), 5.09 (quin, J = 7.0, 1H), 4.25 (br s, 1H), 3.87 (s, 3H), 3.79 (s, 3H), 3.40 (br s, 1H), 3.29 (dd, J = 9.0, 2.0, 1H), 3.15 (d, J = 9.0, 1H), 2.74 (dd, J = 9.0, 1.5, 1H), 2.49 (d, J = 9.0, 1H), 2.24 (s, 3H), 2.16 (s, 3H), 1.85 (br d, J = 9.0, 1H), 1.74 (br d, J = 9.0, 1H), 1.43 (d, J = 7.0, 3H). LC-MS (Method B): R_{T} = 3.56, m/z = 458.9 [M − H]⁻. |
| 245 | 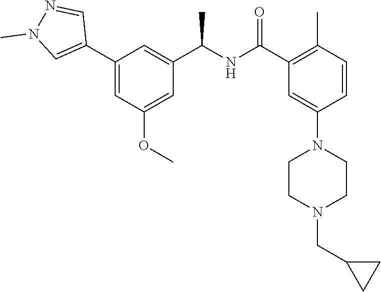 [SMILES: COc1cc(-c2cnn(C)c2)cc([C@@H](C)NC(=O)c2cc(N3CCN(CC4CC4)CC3)ccc2C)c1] | 5-[4-(Cyclopropylmethyl)piperazin-1-yl]-N-[(1R)-1-[3-methoxy-5-(1- methylpyrazol-4-yl)phenyl]ethyl]-2-methyl-benzamide ¹H NMR (500 MHz, DMSO-d₆) δ 8.61 (d, J = 8.0, 1H), 8.11 (s, 1H), 7.83 (s, 1H), 7.18 (br s, 1H), 7.06 (d, J = 8.5, 1H), 6.98 (br s, 1H), 6.91 (dd, J = 8.0, 2.0, 1H), 6.85 (d, J = 2.0, 1H), 6.81 (br s, 1H), 5.09 (quin, J = 7.0, 1H), 3.86 (s, 3H), 3.78 (s, 3H), 3.14-3.13 (m, 4H), 2.66-2.65 (m, 4H), 2.32-2.31 (m, 2H), 2.18 (s, 3H), 1.43 (d, J = 7.0, 3H), 0.88-0.87 (m, 1H), 0.50-0.49 (m, 2H), 0.13-0.12 (m, 2H). LC-MS (Method B): R_{T} = 3.61, m/z = 486.9 [M − H]⁻. |
| 246 | 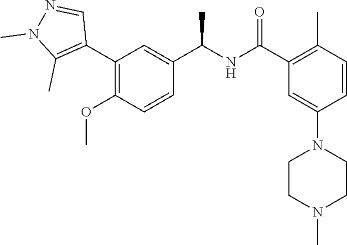 [SMILES: COc1ccc([C@@H](C)NC(=O)c2cc(N3CCN(C)CC3)ccc2C)cc1-c1cnn(C)c1C] | N-[(1R)-1-[3-(1,5-Dimethylpyrazol-4-yl)-4-methoxy-phenyl]ethyl]-2- methyl-5-(4-methylpiperazin-1-yl)benzamide ¹H NMR (500 MHz, DMSO-d₆) δ 8.58 (d, J = 8.5, 1H), 7.36 (s, 1H), 7.26 (dd, J = 8.5, 2.0, 1H), 7.21 (d, J = 2.0, 1H), 7.05 (d, J = 8.5, 1H), 7.01 (d, J = 8.5, 1H), 6.90 (dd, J = 8.5, 2.5, 1H), 6.82 (d, J = 2.5, 1H), 5.09 (quin, J = 7.0, 1H), 3.77 (s, 3H), 3.75 (s, 3H), 3.09-3.08 (m, 4H), 2.45-2.44 (m, 4H), 2.23 (s, 3H), 2.19 (s, 3H), 2.17 (s, 3H), 1.42 (d, J = 7.0, 3H). LC-MS (Method B): R_{T} = 3.29, m/z = 460.9 [M − H]⁻. |
| 247 | 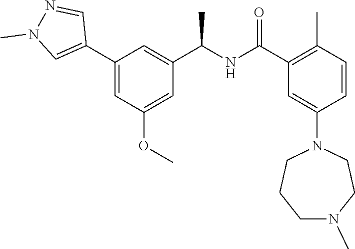 [SMILES: COc1cc(-c2cnn(C)c2)cc([C@@H](C)NC(=O)c2cc(N3CCCN(C)CC3)ccc2C)c1] | N-[(1R)-1-[3-Methoxy-5-(1-methylpyrazol-4-yl)phenyl]ethyl]-2- methyl-5-(4-methyl-1,4-diazepan-1-yl)benzamide ¹H NMR (500 MHz, DMSO-d₆) δ 8.59 (d, J = 8.0, 1H), 8.12 (s, 1H), 7.83 (s, 1H), 7.18 (br s, 1H), 6.99 (m, 2H), 6.82 (br s, 1H), 6.66 (dd, J = 8.5, 2.5, 1H), 6.60 (d, J = 2.5, 1H), 5.09 (quin, J = 7.0, 1H), 3.87 (s, 3H), 3.79 (s, 3H), 3.49-3.48 (m, 2H), 3.41 (t, J = 6.5, 2H), 2.58-2.57 (m, 2H), 2.45-2.44 (m, 2H), 2.25 (s, 3H), 2.15 (s, 3H), 1.88-1.87 (m, 2H), 1.43 (d, J = 7.0, 3H). LC-MS (Method B): R_{T} = 3.59, m/z = 460.9 [M − H]⁻. |
| 248 | 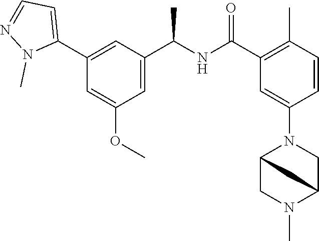 [SMILES: COc1cc(-c2ccnn2C)cc([C@@H](C)NC(=O)c2cc(N3C[C@H]4C[C@@H]3CN4C)ccc2C)c1] | N-[(1R)-1-[3-Methoxy-5-(2-methylpyrazol-3-yl)phenyl]ethyl]-2- methyl-5-[(1R,4R)-5-methyl-2,5-diazabicyclo[2.2.1]heptan-2- yl]benzamide ¹H NMR (500 MHz, DMSO-d₆) δ 8.65 (d, J = 8.0, 1H), 7.47 (br s, 1H), 7.13 (br s, 1H), 7.04 (br s, 1H), 7.00 (d, J = 8.0, 1H), 6.94 (br s, 1H), 6.55 (dd, J = 8.5, 2.5, 1H), 6.48 (d, J = 2.0, 1H), 6.39 (br s, 1H), 5.14 (quin, J = 7.0, 1H), 4.22 (br s, 1H), 3.87 (s, 3H), 3.82 (s, 3H), 3.39 (br s, 1H), 3.28-3.27 (m, 1H), 3.13 (d, J = 9.0, 1H), 2.73 (d, J = 9.0, 1H), 2.44 (br d, J = 9.5, 1H), 2.24 (s, 3H), 2.15 (s, 3H), 1.85 (br d, J = 9.0, 1H), 1.73 (br d, J = 9.0, 1H), 1.45 (d, J = 7.0, 3H). LC-MS (Method B): R_{T} = 3.20, m/z = 458.9 [M − H]⁻. |
| 249 | 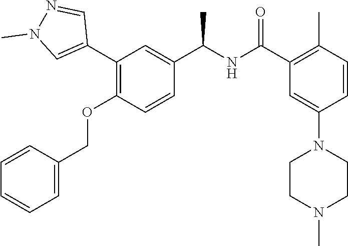 [SMILES: Cc1ccc(N2CCN(C)CC2)cc1C(=O)N[C@H](C)c1ccc(OCc2ccccc2)c(-c2cnn(C)c2)c1] | N-[(1R)-1-[4-Benzyloxy-3-(1-methylpyrazol-4-yl)phenyl]ethyl]-2- methyl-5-(4-methylpiperazin-1-yl)benzamide ¹H NMR (500 MHz, CDCl₃) δ 7.88 (s, 1H), 7.80 (s, 1H), 7.55 (d, J = 2.3, 1H), 7.46-7.42 (m, 2H), 7.41-7.37 (m, 2H), 7.36-7.31 (m, 1H), 7.20-7.16 (m, 1H), 7.10-7.05 (m, 1H), 7.00-6.96 (m, 1H), 6.93-6.90 (m, 1H), 6.89-6.84 (m, 1H), 5.95-5.90 (m, 1H), 5.34-5.27 (m, 1H), 5.15 (s, 2H), 3.90 (s, 3H), 3.19-3.11 (m, 4H), 2.59-2.52 (m, 4H), 2.35 (s, 3H), 2.31 (s, 3H), 1.56 (d, J = 8.1, 3H). LC-MS (Method A): R_{T} = 4.62, m/z = 524.8 [M + H]⁺. |
| 250 | 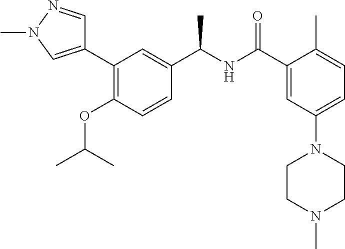 [SMILES: Cc1ccc(N2CCN(C)CC2)cc1C(=O)N[C@H](C)c1ccc(OC(C)C)c(-c2cnn(C)c2)c1] | N-[(1R)-1-[4-isopropoxy-3-(1-methylpyrazol-4-yl)phenyl]ethyl]-2- methyl-5-(4-methylpiperazin-1-yl)benzamide ¹H NMR (500 MHz, CDCl₃) δ 7.90 (s, 1H), 7.84 (s, 1H), 7.51 (d, J = 2.1, 1H), 7.17 (dd, J = 2.1, 8.5, 1H), 7.07 (d, J = 8.2, 1H), 6.92-6.91 (m, 2H), 6.89-6.84 (m, 1H), 5.92 (br d, J = 8.2, 1H), 5.35-5.23 (m, 1H), 4.63 (td, J = 6.0, 12.1, 1H), 3.94 (s, 3H), 3.20-3.10 (m, 4H), 2.58-2.53 (m, 4H), 2.34 (s, 3H), 2.32 (s, 3H), 1.60 (d, J = 6.7, 3H), 1.39 (d, J = 6.1, 6H). LC-MS (Method B): R_{T} = 3.47, m/z = 475.0 [M − H]⁻. |
| 251 | 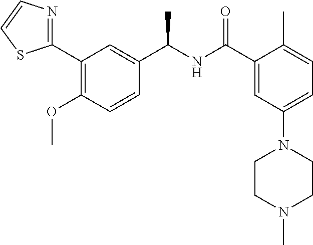 [SMILES: COc1ccc([C@@H](C)NC(=O)c2cc(N3CCN(C)CC3)ccc2C)cc1-c1nccs1] | N-[(1R)-1-(4-Methoxy-3-thiazol-2-yl-phenyl)ethyl]-2-methyl-5-(4- methylpiperazin-1-yl)benzamide ¹H NMR (500 MHz, CDCl₃) δ 8.43 (d, J = 2.1, 1H), 7.90 (d, J = 3.4, 1H), 7.51-7.33 (m, 2H), 7.12-6.98 (m, 2H), 6.93 (d, J = 2.4, 1H), 6.91-6.81 (m, 1H), 6.01 (br d, J = 7.9, 1H), 5.38-5.30 (m, 1H), 4.04 (s, 3H), 3.21-3.13 (m, 4H), 2.59-2.53 (m, 4H), 2.34 (s, 3H), 2.32-2.30 (m, 3H), 1.64 (d, J = 7.0, 3H). LC-MS (Method B): R_{T} = 3.59, m/z = 449.8 [M − H]⁻. |
| 252 | 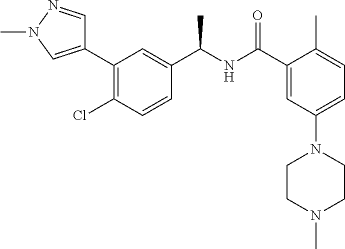 [SMILES: Cc1ccc(N2CCN(C)CC2)cc1C(=O)N[C@H](C)c1ccc(Cl)c(-c2cnn(C)c2)c1] | N-[(1R)-1-[4-Chloro-3-(1-methylpyrazol-4-yl)phenyl]ethyl]-2-methyl- 5-(4-methylpiperazin-1-yl)benzamide ¹H NMR (500 MHz, CDCl₃) δ 7.79 (s, 1H), 7.79-7.78 (m, 1H), 7.45 (d, J = 2.1, 1H), 7.44-7.40 (m, 1H), 7.19 (dd, J = 2.1, 8.2, 1H), 7.08 (d, J = 8.2, 1H), 6.91 (d, J = 2.4, 1H), 6.90-6.86 (m, 1H), 5.98 (br d, J = 7.9, 1H), 5.34-5.26 (m, 1H), 3.96 (s, 3H), 3.20-3.11 (m, 4H), 2.60-2.52 (m, 4H), 2.34 (s, 3H), 2.32-2.29 (m, 3H), 1.58 (d, J = 7.0, 3H). LC-MS (Method B): R_{T} = 3.52, m/z = 450.8 [M − H]⁻. |
| 253 | 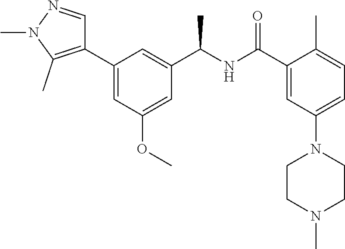 [SMILES: COc1cc(-c2cnn(C)c2C)cc([C@@H](C)NC(=O)c2cc(N3CCN(C)CC3)ccc2C)c1] | N-[(1R)-1-[3-(1,5-Dimethylpyrazol-4-yl)-5-methoxy-phenyl]ethyl]-2- methyl-5-(4-methylpiperazin-1-yl)benzamide hydrochloride salt ¹H NMR (500 MHz, DMSO-d₆) δ 8.69 (br d, J = 8.2, 1H), 7.57 (s, 1H), 7.13 (br d, J = 8.2, 1H), 7.04-6.95 (m, 2H), 6.94-6.89 (m, 1H), 6.89-6.84 (m, 1H), 6.84-6.74 (m, 1H), 5.12 (s, 1H), 3.87-3.71 (m, 8H), 3.47 (br d, J = 9.8, 2H), 3.19-3.06 (m, 4H), 2.85-2.76 (m, 3H), 2.43-2.33 (m, 3H), 2.21 (s, 3H), 1.45 (br d, J = 7.0, 3H). LC-MS (Method B): R_{T} = 3.48, m/z = 460.9 [M − H]⁻. |
| 254 | 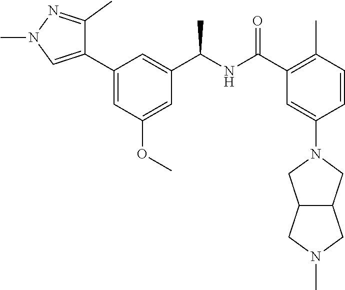 [SMILES: COc1cc(-c2cn(C)nc2C)cc([C@@H](C)NC(=O)c2cc(N3CC4CN(C)CC4C3)ccc2C)c1] | N-[(1R)-1-[3-(1,3-Dimethylpyrazol-4-yl)-5-methoxy-phenyl]ethyl]-2- methyl-5-(2-methyl-1,3,3a,4,6,6a-hexahydropyrrolo[3,4-c]pyrrol-5- yl)benzamide ¹H NMR (500 MHz, CDCl₃) δ 7.43 (s, 1H), 7.06-7.00 (m, 1H), 7.00-6.96 (m, 1H), 6.82 (br d, J = 7.6, 2H), 6.66 (d, J = 2.1, 1H), 6.64-6.58 (m, 1H), 5.95 (br d, J = 7.9, 1H), 5.31 (quin, J = 7.1, 1H), 3.87 (s, 3H), 3.84 (s, 3H), 3.34-3.25 (m, 2H), 3.16 (br d, J = 9.5, 2H), 2.98-2.90 (m, 2H), 2.90-2.68 (m, 2H), 2.45-2.23 (m, 11H), 1.60 (d, J = 6.7, 3H). LC-MS (Method B): R_{T} = 3.90, m/z = 486.9 [M − H]⁻. |
| 255 | 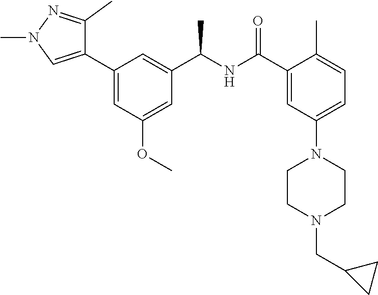 [SMILES: COc1cc(-c2cn(C)nc2C)cc([C@@H](C)NC(=O)c2cc(N3CCN(CC4CC4)CC3)ccc2C)c1] | 5-[4-(Cyclopropylmethyl)piperazin-1-yl]-N-[(1R)-1-[3-(1,3- dimethylpyrazol-4-yl)-5-methoxy-phenyl]ethyl]-2-methyl-benzamide hydrochloride salt ¹H NMR (500 MHz, DMSO-d₆) δ 11.28-11.14 (m, 1H), 8.72 (d, J = 8.2, 1H), 7.93 (s, 1H), 7.13 (d, J = 8.5, 1H), 7.05 (s, 1H), 7.02-6.96 (m, 1H), 6.93 (d, J = 2.4, 1H), 6.86 (s, 1H), 6.84-6.81 (m, 1H), 5.16-5.06 (m, 1H), 3.81-3.76 (m, 4H), 3.61-3.54 (m, 6H), 3.25-3.09 (m, 4H), 3.09-2.98 (m, 2H), 2.32 (s, 3H), 2.20 (s, 3H), 1.45 (d, J = 7.0, 3H), 1.22-1.14 (m, 1H), 0.73-0.59 (m, 2H), 0.43 (q, J = 4.9, 2H). LC-MS (Method B): R_{T} = 3.72, m/z = 500.9 [M − H]⁻. |
| 256 | 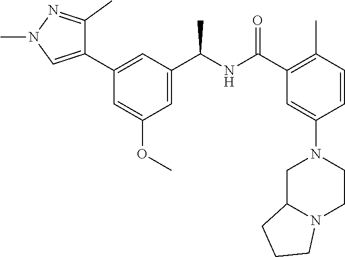 [SMILES: COc1cc(-c2cn(C)nc2C)cc([C@@H](C)NC(=O)c2cc(N3CCN4CCCC4C3)ccc2C)c1] | 5-(3,4,6,7,8,8a-Hexahydro-1H-pyrrolo[1,2-a]pyrazin-2-yl)-N-[(1R)-1- [3-(1,3-dimethylpyrazol-4-yl)-5-methoxy-phenyl]ethyl]-2-methyl- benzamide ¹H NMR (500 MHz, CDCl₃) δ 7.43 (s, 1H), 7.08 (d, J = 8.2, 1H), 6.98 (s, 1H), 6.96-6.92 (m, 1H), 6.89 (dd, J = 2.6, 8.4, 1H), 6.85-6.80 (m, 2H), 5.94 (br d, J = 7.9, 1H), 5.37-5.24 (m, 1H), 3.87 (s, 3H), 3.84 (s, 3H), 3.68 (br d, J = 9.8, 1H), 3.54 (br d, J = 11.9, 1H), 3.21-3.05 (m, 2H), 2.88 (dt, J = 3.1, 11.6, 1H), 2.51 (t, J = 10.7, 1H), 2.43-2.25 (m, 7H), 2.24-2.09 (m, 2H), 1.92-1.83 (m, 2H), 1.82-1.72 (m, 1H), 1.60 (d, J = 7.0, 3H), 1.57-1.39 (m, 1H). LC-MS (Method B): R_{T} = 3.47, m/z = 486.9 [M − H]⁻. |
| 257 | 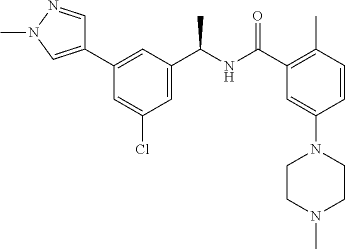 [SMILES: Cc1ccc(N2CCN(C)CC2)cc1C(=O)N[C@H](C)c1cc(Cl)cc(-c2cnn(C)c2)c1] | N-[(1R)-1-[3-Chloro-5-(1-methylpyrazol-4-yl)phenyl]ethyl]-2-methyl- 5-(4-methylpiperazin-1-yl)benzamide ¹H NMR (500 MHz, CDCl₃) δ 7.73 (s, 1H), 7.67-7.56 (m, 1H), 7.35 (s, 1H), 7.26 (s, 1H), 7.23-7.16 (m, 1H), 7.16-7.02 (m, 1H), 6.99-6.81 (m, 2H), 5.98 (br d, J = 7.3, 1H), 5.32-5.26 (m, 1H), 3.94 (s, 3H), 3.16 (br s, 4H), 2.56 (br s, 4H), 2.36-2.30 (m, 6H), 1.63-1.55 (m, 3H). LC-MS (Method B): R_{T} = 3.30, m/z = 452.6 [M − H]⁻. |

### Example 258: 5-(2,7-Diazaspiro[3.5]nonan-2-yl)-N-[(1R)-1-[4-methoxy-3-(1-methylpyrazol-4-yl)phenyl]ethyl]-2-methyl-benzamide

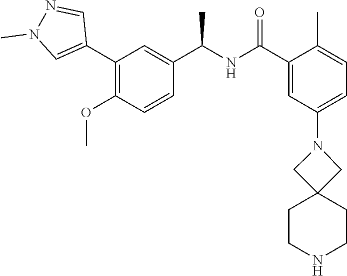 [SMILES: COc1ccc([C@@H](C)NC(=O)c2cc(N3CC4(CCNCC4)C3)ccc2C)cc1-c1cnn(C)c1]

Using General Procedure 4 with tert-butyl 2-[3-[[(1R)-1-[4-methoxy-3-(1-methylpyrazol-4-yl)phenyl]ethyl]carbamoyl]-4-methyl-phenyl]-2,7-diazaspiro[3.5]nonane-7-carboxylate (166 mg, 289 μmol)—prepared in a similar manner to N-[(1R)-1-[2-methoxy-5-(1-methylpyrazol-4-yl)phenyl]ethyl]-2-methyl-5-(4-methylpiperazin-1-yl)benzamide (Example 239)—gave 5-(2,7-diazaspiro[3.5]nonan-2-yl)-N-[(1R)-1-[4-methoxy-3-(1-methylpyrazol-4-yl)phenyl]ethyl]-2-methyl-benzamide (65 mg, 47%) as a white solid. ¹H NMR (500 MHz, CDCl₃) δ 7.86 (s, 1H), 7.85-7.83 (m, 1H), 7.51 (d, J=2.1, 1H), 7.21 (dd, J=2.3, 8.4, 1H), 7.02 (d, J=8.2, 1H), 6.97-6.89 (m, 1H), 6.43 (d, J=2.4, 1H), 6.42-6.37 (m, 1H), 5.91 (br d, J=7.3, 1H), 5.34-5.27 (m, 1H), 3.94 (s, 3H), 3.90 (s, 3H), 3.56 (s, 3H), 2.87-2.72 (m, 4H), 1.74 (br t, J=5.3, 4H), 1.60 (d, J=7.0, 7H). LC-MS (Method B): R_{T}=4.69, m/z=472.9 [M−H]⁻.

### Further Examples

The following examples were prepared in a similar manner to 5-(2,7-diazaspiro[3.5]nonan-2-yl)-N-[(1R)-1-[4-methoxy-3-(1-methylpyrazol-4-yl)phenyl]ethyl]-2-methyl-benzamide (Example 258).

| Example | Structure | Name and Analytical Data |
|---|---|---|
| 259 | 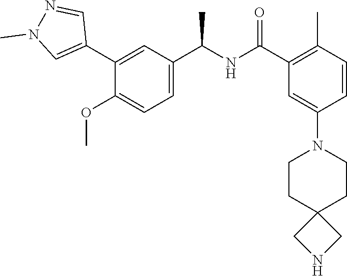 [SMILES: COc1ccc([C@@H](C)NC(=O)c2cc(N3CCC4(CC3)CNC4)ccc2C)cc1-c1cnn(C)c1] | 5-(2,7-Diazaspiro[3.5]nonan-7-yl)-N- [(1R)-1-[4-methoxy-3-(1-methylpyrazol- 4-yl)phenyl]ethyl]-2-methyl-benzamide ¹H NMR (500 MHz, DMSO-d₆) δ 8.56 (d, J = 8.0, 1H), 8.08 (s, 1H), 7.85 (s, 1H), 7.63 (d, J = 2.0, 1H), 7.20 (dd, J = 8.0, 2.0, 1H), 7.04 (d, J = 8.5, 1H), 7.01 (d, J = 8.5, 1H), 6.90 (dd, J = 8.5, 2.5, 1H), 6.84 (d, J = 2.5, 1H), 5.10 (quin, J = 7.0, 1H), 3.88 (s, 3H), 3.86 (s, 3H), 3.22 (br s, 4H), 3.03- 3.02 (m, 4H), 2.17 (s, 3H), 1.77-1.76 (m, 4H), 1.43 (d, J = 7.0, 3H). LC-MS (Method B): R_{T} = 4.87, m/z = 472.9 [M − H]⁻. |
| 260 | 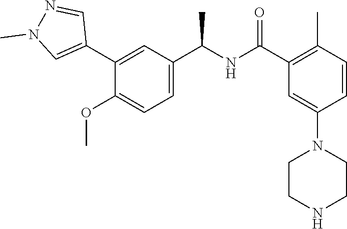 [SMILES: COc1ccc([C@@H](C)NC(=O)c2cc(N3CCNCC3)ccc2C)cc1-c1cnn(C)c1] | N-[(1R)-1-[4-Methoxy-3-(1- methylpyrazol-4-yl)phenyl]ethyl]-2- methyl-5-piperazin-1-yl-benzamide ¹H NMR (500 MHz, DMSO-d₆) δ 8.57 (d, J = 8.5, 1H), 8.08 (s, 1H), 7.85 (s, 1H), 7.63 (d, J = 2.0, 1H), 7.20 (dd, J = 8.5, 2.5, 1H), 7.05 (d, J = 8.0, 1H), 7.02 (d, J = 8.5, 1H), 6.88 (dd, J = 8.5, 2.5, 1H), 6.82 (d, J = 2.5, 1H), 5.10 (quin, J = 7.0, 1H), 3.88 (s, 3H), 3.86 (s, 3H), 3.00-2.99 (m, 4H), 2.82-2.81 (m, 4H), 2.17 (s, 3H), 1.43 (d, J = 7.0, 3H). LC-MS (Method B): R_{T} = 3.15, m/z = 432.8 [M − H]⁻. |
| 261 | 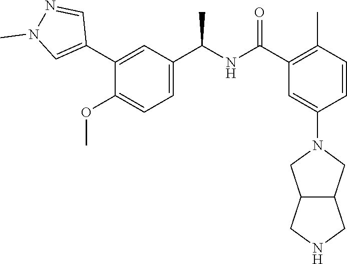 [SMILES: COc1ccc([C@@H](C)NC(=O)c2cc(N3CC4CNCC4C3)ccc2C)cc1-c1cnn(C)c1] | 5-(2,3,3a,4,6,6a-Hexahydro-1H- pyrrolo[3,4-c]pyrrol-5-yl)-N-[(1R)-1-[4- methoxy-3-(1-methylpyrazol-4- yl)phenyl]ethyl]-2-methyl-benzamide ¹H NMR (500 MHz, DMSO-d₆) δ 8.53 (d, J = 8.5, 1H), 8.07 (s, 1H), 7.85 (s, 1H), 7.64 (d, J = 2.0, 1H), 7.20 (dd, J = 8.5, 2.0, 1H), 7.01 (d, J = 8.5, 1H), 6.60 (dd, J = 8.5, 2.5, 1H), 6.56 (d, J = 2.5, 1H), 5.10 (quin, J = 7.0, 1H), 3.88 (s, 3H), 3.86 (s, 3H), 3.01-3.00 (m, 2H), 2.96-2.95 (m, 2H), 2.79- 2.78 (m, 2H), 2.62-2.61 (m, 2H), 2.16 (s, 3H), 1.42 (d, J = 7.0, 3H). LC-MS (Method B): R_{T} = 4.42, m/z = 458.9 [M − H]⁻. |
| 262 | 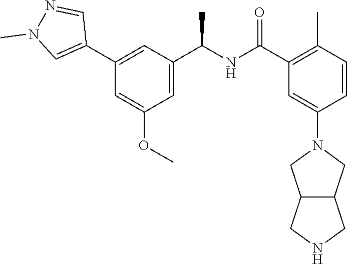 [SMILES: COc1cc(-c2cnn(C)c2)cc([C@@H](C)NC(=O)c2cc(N3CC4CNCC4C3)ccc2C)c1] | 5-(2,3,3a,4,6,6a-Hexahydro-1H- pyrrolo[3,4-c]pyrrol-5-yl)-N-[(1R)-1-[3- methoxy-5-(1-methylpyrazol-4- yl)phenyl]ethyl]-2-methyl-benzamide ¹H NMR (500 MHz, DMSO-d₆) δ 8.58 (d, J = 8.5, 1H), 8.12 (s, 1H), 7.83 (s, 1H), 7.19 (br s, 1H), 7.02 (d, J = 8.5, 1H), 6.99 (br s, 1H), 6.82 (br s, 1H), 6.60 (dd, J = 8.0, 2.5, 1H), 6.57 (d, J = 2.5, 1H), 5.09 (quin, J = 7.0, 1H), 3.87 (s, 3H), 3.79 (s, 3H), 2.99-2.98 (m, 2H), 2.93-2.92 (m, 2H), 2.78-2.77 (m, 2H), 2.59-2.58 (m, 2H), 2.16 (s, 3H), 1.43 (d, J = 7.0, 3H). LC-MS (Method B): R_{T} = 4.45, m/z = 458.8 [M − H]⁻. |
| 263 | 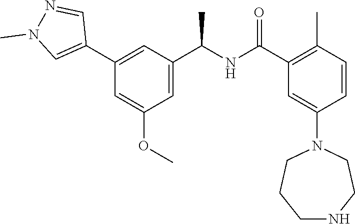 [SMILES: COc1cc(-c2cnn(C)c2)cc([C@@H](C)NC(=O)c2cc(N3CCCNCC3)ccc2C)c1] | 5-(1,4-Diazepan-1-yl)-N-[(1R)-1-[3- methoxy-5-(1-methylpyrazol-4- yl)phenyl]ethyl]-2-methyl-benzamide ¹H NMR (500 MHz, DMSO-d₆) δ 8.58 (d, J = 8.0, 1H), 8.11 (s, 1H), 7.82 (s, 1H), 7.19 (br s, 1H), 6.99-6.98 (m, 2H), 6.82 (br s, 1H), 6.66 (dd, J = 8.0, 2.5, 1H), 6.60 (d, J = 2.5, 1H), 5.09 (quin, J = 7.0, 1H), 3.86 (s, 3H), 3.79 (s, 3H), 3.51-3.50 (m, 2H), 3.44-3.43 (m, 2H), 2.83-2.82 (m, 2H), 2.63- 2.62 (m, 2H), 2.15 (s, 3H), 1.76-1.75 (m, 2H), 1.42 (d, J = 7.0, 3H). LC-MS (Method B): R_{T} = 3.99, m/z = 446.8 [M − H]⁻. |
| 264 | 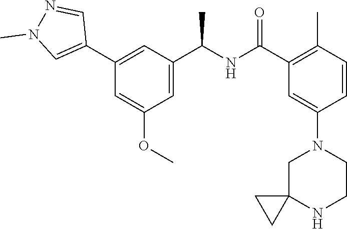 [SMILES: COc1cc(-c2cnn(C)c2)cc([C@@H](C)NC(=O)c2cc(N3CCNC4(CC4)C3)ccc2C)c1] | 5-(4,7-Diazaspiro[2.5]octan-7-yl)-N- [(1R)-1-[3-methoxy-5-(1-methylpyrazol- 4-yl)phenyl]ethyl]-2-methyl-benzamide ¹H NMR (500 MHz, DMSO-d₆) δ 8.60 (d, J = 8.0, 1H), 8.12 (s, 1H), 7.83 (s, 1H), 7.18 (br s, 1H), 7.05 (d, J = 8.0, 1H), 6.99- 6.98 (m, 1H), 6.87 (dd, J = 8.5, 2.5, 1H), 6.82-6.81 (m, 1H), 6.80 (d, J = 2.5, 1H), 5.09 (quin, J = 7.0, 1H), 3.87 (s, 3H), 3.79 (s, 3H), 3.04-3.03 (m, 2H), 2.90-2.89 (m, 4H), 2.18 (s, 3H), 1.43 (d, J = 7.0, 3H), 0.50 (br s, 4H). LC-MS (Method B): R_{T} = 3.22, m/z = 458.8 [M − H]⁻. |
| 265 | 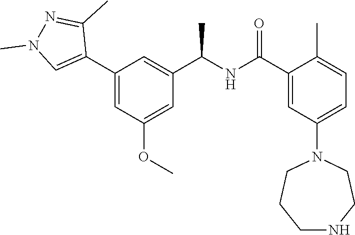 [SMILES: COc1cc(-c2cn(C)nc2C)cc([C@@H](C)NC(=O)c2cc(N3CCCNCC3)ccc2C)c1] | 5-(1,4-Diazepan-1-yl)-N-[(1R)-1-[3-(1,3- dimethylpyrazol-4-yl)-5-methoxy- phenyl]ethyl]-2-methyl-benzamide ¹H NMR (500 MHz, CDCl₃) δ 7.42 (s, 1H), 7.04-6.99 (m, J = 8.5, 1H), 6.99-6.96 (m, 1H), 6.87-6.77 (m, 2H), 6.69 (br s, 1H), 6.67-6.62 (m, 1H), 5.95 (br d, J = 7.6, 1H), 5.37-5.26 (m, 1H), 3.87 (s, 3H), 3.83 (s, 3H), 3.60-3.45 (m, 4H), 2.99 (br t, J = 5.0, 2H), 2.80 (br t, J = 5.5, 2H), 2.39 (s, 3H), 2.29 (s, 3H), 1.94-1.78 (m, 2H), 1.60 (br d, J = 6.7, 3H). LC-MS (Method B): R_{T} = 3.97, m/z = 460.9 [M − H]⁻. |
| 266 | 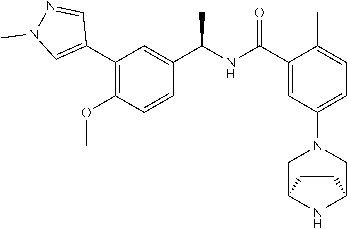 [SMILES: COc1ccc([C@@H](C)NC(=O)c2cc(N3C[C@H]4CC[C@@H](C3)N4)ccc2C)cc1-c1cnn(C)c1] | 5-[(1S,5R)-3,8-Diazabicyclo[3.2.1]octan- 3-yl]-N-[(1R)-1-[4-methoxy-3-(1- methylpyrazol-4-yl)phenyl]ethyl]-2- methyl-benzamide hydrochloride salt ¹H NMR (500 MHz, DMSO-d₆) δ 9.57-9.35 (m, 1H), 8.61 (d, J = 8.5, 1H), 8.09 (s, 1H), 7.87 (s, 1H), 7.64 (d, J = 2.1, 1H), 7.19 (br d, J = 1.8, 1H), 7.08 (d, J = 8.2, 1H), 7.01 (d, J = 8.2, 1H), 6.88 (s, 1H), 6.80 (d, J = 2.4, 1H), 5.25-4.82 (m, 1H), 4.10 (br s, 2H), 3.88 (s, 3H), 3.87-3.84 (m, 3H), 3.57-3.56 (m, 2H), 3.11 (br d, J = 11.9, 2H), 2.17 (s, 3H), 2.05-1.81 (m, 4H), 1.43 (d, J = 7.0, 3H). LC- MS (Method B): R_{T} = 3.65, m/z = 458.9 [M − H]⁻. |
| 267 | 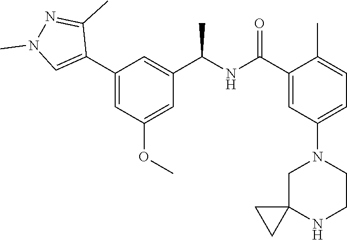 [SMILES: COc1cc(-c2cn(C)nc2C)cc([C@@H](C)NC(=O)c2cc(N3CCNC4(CC4)C3)ccc2C)c1] | 5-(4,7-Diazaspiro[2.5]octan-7-yl)-N- [(1R)-1-[3-(1,3-dimethylpyrazol-4-yl)-5- methoxy-phenyl]ethyl]-2-methyl- benzamide ¹H NMR (500 MHz, CDCl₃) δ 7.43 (s, 1H), 7.08 (br d, J = 8.2, 1H), 6.98 (s, 1H), 6.89 (br s, 1H), 6.85-6.79 (m, 3H), 5.95 (br d, J = 7.6, 1H), 5.31 (quin, J = 7.0, 1H), 3.87 (s, 3H), 3.84 (s, 3H), 3.09 (br s, 4H), 2.91 (s, 2H), 2.39 (s, 3H), 2.33 (s, 3H), 1.69-1.54 (m, 3H), 0.69 (brs, 2H), 0.58 (brs, 2H). LC- MS (Method B): R_{T} = 3.12, m/z = 472.9 [M − H]⁻. |
| 268 | 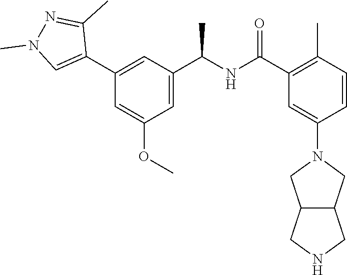 [SMILES: COc1cc(-c2cn(C)nc2C)cc([C@@H](C)NC(=O)c2cc(N3CC4CNCC4C3)ccc2C)c1] | 5-(2,3,3a,4,6,6a-Hexahydro-1H- pyrrolo[3,4-c]pyrrol-5-yl)-N-[(1R)-1-[3- (1,3-dimethylpyrazol-4-yl)-5-methoxy- phenyl]ethyl]-2-methyl-benzamide ¹H NMR (500 MHz, CDCl₃) δ 7.43 (s, 1H), 7.04 (d, J = 8.2, 1H), 7.01-6.96 (m, 1H), 6.90-6.75 (m, 2H), 6.64 (d, J = 2.1, 1H), 6.62-6.57 (m, 1H), 5.97 (br d, J = 7.9, 1H), 5.31 (quin, J = 7.2, 1H), 3.87 (s, 3H), 3.84 (s, 3H), 3.41-3.24 (m, 2H), 3.19-3.10 (m, 4H), 2.89 (br s, 2H), 2.86-2.76 (m, 2H), 2.40 (s, 3H), 2.38-2.24 (m, 3H), 1.60 (d, J = 7.0, 3H). LC-MS (Method B): R_{T} = 4.64, m/z = 472.9 [M − H]⁻. |
| 269 | 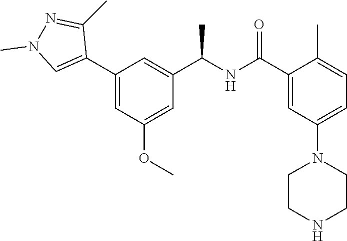 [SMILES: COc1cc(-c2cn(C)nc2C)cc([C@@H](C)NC(=O)c2cc(N3CCNCC3)ccc2C)c1] | N-[(1R)-1-[3-(1,3-Dimethylpyrazol-4-yl)- 5-methoxy-phenyl]ethyl]-2-methyl-5- piperazin-1-yl-benzamide ¹H NMR (500 MHz, CDCl₃) δ 7.43 (s, 1H), 7.09 (br d, J = 8.2, 1H), 7.01-6.77 (m, 5H), 5.96 (br d, J = 7.3, 1H), 5.38-5.24 (m, 1H), 3.87 (s, 3H), 3.84 (s, 3H), 3.23-2.93 (m, 8H), 2.48-2.20 (m, 6H), 1.60 (br d, J = 6.4, 3H). LC-MS (Method B): R_{T} = 3.53 m/z = 446.8 [M − H]⁻. |
| 270 | 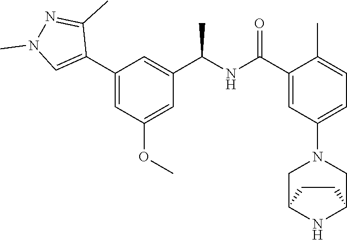 [SMILES: COc1cc(-c2cn(C)nc2C)cc([C@@H](C)NC(=O)c2cc(N3C[C@H]4CC[C@@H](C3)N4)ccc2C)c1] | 5-[(1S,5R)-3,8-Diazabicyclo[3.2.1]octan- 3-yl]-N-[(1R)-1-[3-(1,3-dimethylpyrazol- 4-yl)-5-methoxy-phenyl]ethyl]-2-methyl- benzamide ¹H NMR (500 MHz, CDCl₃) δ 7.43 (s, 1H), 7.05 (d, J = 8.2, 1H), 7.01-6.95 (m, 1H), 6.84-6.77 (m, 3H), 6.77-6.71 (m, 1H), 5.95 (br d, J = 7.9, 1H), 5.38-5.24 (m, 1H), 3.87 (s, 3H), 3.84 (s, 3H), 3.62 (br s, 2H), 3.38 (br d, J = 9.8, 2H), 2.85 (br d, J = 9.8, 2H), 2.40 (s, 3H), 2.31 (s, 3H), 1.89-1.75 (m, 4H), 1.60 (d, J = 6.7, 3H). LC-MS (Method B): R_{T} = 3.79, m/z = 472.9 [M − H]⁻. |
| 271 | 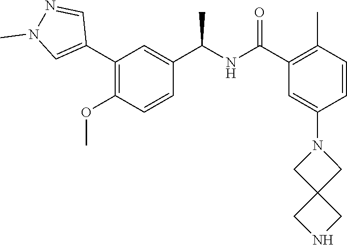 [SMILES: COc1ccc([C@@H](C)NC(=O)c2cc(N3CC4(CNC4)C3)ccc2C)cc1-c1cnn(C)c1] | 5-(2,6-Diazaspiro[3.3]heptan-2-yl)-N- [(1R)-1-[4-methoxy-3-(1-methylpyrazol- 4-yl)phenyl]ethyl]-2-methyl-benzamide ¹H NMR (500 MHz, CDCl₃) δ 7.90 (s, 1H), 7.83 (s, 1H), 7.57-7.49 (m, 1H), 7.23 (dd, J = 2.1, 8.5, 1H), 7.05-6.99 (m, 1H), 6.97- 6.91 (m, 1H), 6.41-6.30 (m, 2H), 5.29-5.22 (m, 1H), 3.98-3.62 (m, 14H), 2.29 (s, 3H), 1.61 (d, J = 6.9, 3H), 3H), 2.15 (s, 3H), 1.40 (d, J = 6.9, 3H). LC-MS (Method A): R_{T} = 3.04, m/z = 446.6 [M + H]⁺. |
| 272 | 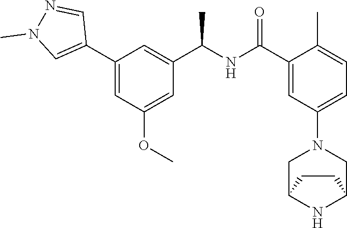 [SMILES: COc1cc(-c2cnn(C)c2)cc([C@@H](C)NC(=O)c2cc(N3C[C@H]4CC[C@@H](C3)N4)ccc2C)c1] | 5-[(1R,5S)-3,8-diazabicyclo[3.2.1]octan- 3-yl]-N-[(1R)-1-[3-methoxy-5-(1-methyl- 1H-pyrazol-4-yl)phenyl]ethyl]-2- methylbenzamide ¹H NMR (500 MHz, DMSO-d₆) δ 8.59 (br d, J = 8.2, 1H), 8.12 (s, 1H), 7.83 (s, 1H), 7.18 (s, 1H), 7.02 (d, J = 8.5, 1H), 6.99 (s, 1H), 6.81 (s, 1H), 6.76 (dd, J = 2.3, 8.5, 1H), 6.71 (d, J = 2.3, 1H), 5.13-5.05 (m, 1H), 3.87 (s, 3H), 3.79 (s, 3H), 3.48 (br s, 2H), 3.34 (br s, 2H), 2.71 (br s, 2H), 2.24-2.16 (m, 4H), 1.67 (br s, 3H), 1.43 (d, J = 6.9, 3H). LC-MS (Method B): R_{T} = 3.79, m/z = 458.9 [M − H]⁻. |

### Example 273: 5-[4-(2-Hydroxyethyl)piperazin-1-yl]-N-[(1R)-1-[4-methoxy-3-(1-methylpyrazol-4-yl)phenyl]ethyl]-2-methyl-benzamide

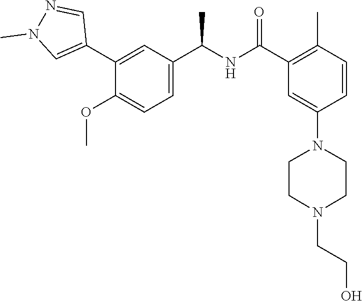 [SMILES: COc1ccc([C@@H](C)NC(=O)c2cc(N3CCN(CCO)CC3)ccc2C)cc1-c1cnn(C)c1]

Using General Procedure 4 with 5-[4-[2-[tert-butyl(dimethyl)silyl]oxyethyl]piperazin-1-yl]-N-[(1R)-1-[4-methoxy-3-(1-methylpyrazol-4-yl)phenyl]ethyl]-2-methyl-benzamide (84 mg, 142 μmol)—prepared in a similar manner to N-[(1R)-1-[2-methoxy-5-(1-methylpyrazol-4-yl)phenyl]ethyl]-2-methyl-5-(4-methylpiperazin-1-yl)benzamide (Example 239)—with purification by FCC (eluting with 0-100% MeOH in ethyl acetate) gave 5-[4-(2-hydroxyethyl)piperazin-1-yl]-N-[(1R)-1-[4-methoxy-3-(1-methylpyrazol-4-yl)phenyl]ethyl]-2-methyl-benzamide (22 mg, 31%) as a white crystalline solid. ¹H NMR (500 MHz, DMSO-d₆) δ 8.57 (d, J=8.0, 1H), 8.08 (s, 1H), 7.85 (s, 1H), 7.63 (d, J=2.0, 1H), 7.20 (dd, J=8.0, 2.0, 1H), 7.06 (d, J=8.5, 1H), 7.02 (d, J=8.5, 1H), 6.90 (dd, J=8.5, 2.5, 1H), 6.83 (d, J=2.5, 1H), 5.11 (quin, J=7.0, 1H), 4.42 (t, J=6.0, 1H), 3.88 (s, 3H), 3.86 (s, 3H), 3.53 (q, J=6.0, 2H), 3.09 (m, 4H), 2.54 (m, 4H), 2.43 (t, J=6.0, 2H), 2.17 (s, 3H), 1.43 (d, J=7.0, 3H). LC-MS (Method B): R_{T}=3.13, m/z=477.0 [M−H]⁻.

### Example 274: 5-[(1R,5S)-8-(Cyclopropylmethyl)-3,8-diazabicyclo[3.2.1]octan-3-yl]-N-[(1R)-1-[3-methoxy-5-(1-methylpyrazol-4-yl)phenyl]ethyl]-2-methyl-benzamide

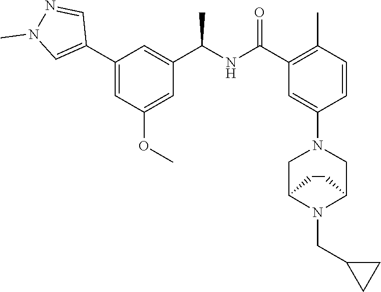 [SMILES: COc1cc(-c2cnn(C)c2)cc([C@@H](C)NC(=O)c2cc(N3C[C@H]4CC[C@@H](C3)N4CC3CC3)ccc2C)c1]

Bromomethylcyclopropane (14 mg, 101 μmol) was added to a solution of 5-[(1S,5R)-3,8-diazabicyclo[3.2.1]octan-3-yl]-N-[(1R)-1-[3-methoxy-5-(1-methylpyrazol-4-yl)phenyl]ethyl]-2-methyl-benzamide (46 mg, 101 μmol) (Example 270) and potassium carbonate (15 mg, 111 μmol) in DMF (5 mL) and the reaction mixture heated to 70° C. for 1h. A further portion of (bromomethyl)cyclopropane (27 mg, 201 μmol) was then added and the reaction mixture allowed to stir for a further 2h. The reaction mixture was allowed to cool to RT and water (75 mL) and ethyl acetate (75 mL) added. The phases were separated, and the aqueous phase extracted with ethyl acetate (75 mL). The combined organic phases were washed with brine (120 mL), dried over sodium sulfate and the solvent removed in vacuo. Purification by FCC (eluting with 0-100% MeOH in ethyl acetate) gave 5-[(1R,5S)-8-(cyclopropylmethyl)-3,8-diazabicyclo[3.2.1]octan-3-yl]-N-[(1R)-1-[3-methoxy-5-(1-methylpyrazol-4-yl)phenyl]ethyl]-2-methyl-benzamide (24 mg, 44%) as a pale yellow solid. ¹H NMR (500 MHz, DMSO-d₆) δ 8.58 (d, J=8.5, 1H), 8.11 (s, 1H), 7.82 (s, 1H), 7.18 (s, 1H), 7.02 (d, J=8.5, 1H), 6.98 (br s, 1H), 6.81 (br s, 1H), 6.76 (dd, J=8.5, 2.5, 1H), 6.72 (d, J=2.5, 1H), 5.08 (quin, J=7.0, 1H), 3.86 (s, 3H), 3.78 (s, 3H), 3.42 (br s, 2H), 2.80 (br t, J=8.0, 2H), 2.24 (d, J=6.0, 2H), 2.16 (s, 3H), 1.85 (m, 2H), 1.60 (m, 2H), 1.42 (d, J=7.0, 3H), 1.24 (br s, 2H), 0.85 (m, 1H), 0.45 (m, 2H), 0.11 (m, 2H). LC-MS (Method B): R_{T}=3.74, m/z=512.9 [M−H]⁻.

### Example 275: N-[(1R)-1-[3-Hydroxy-5-(1-methylpyrazol-4-yl)phenyl]ethyl]-2-methyl-5-(4-methylpiperazin-1-yl)benzamide

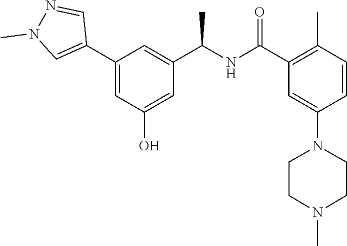 [SMILES: Cc1ccc(N2CCN(C)CC2)cc1C(=O)N[C@H](C)c1cc(O)cc(-c2cnn(C)c2)c1]

Using General Procedure 5 with N-[(1R)-1-[3-benzyloxy-5-(1-methylpyrazol-4-yl)phenyl]ethyl]-2-methyl-5-(4-methylpiperazin-1-yl)benzamide (520 mg, 993 μmol)—prepared in a similar manner to N-[(1R)-1-[2-methoxy-5-(1-methylpyrazol-4-yl)phenyl]ethyl]-2-methyl-5-(4-methylpiperazin-1-yl)benzamide (Example 239)—and palladium hydroxide, 20% on carbon (140 mg, 99.3 μmol), with purification by FCC (eluting with 0-100% MeOH in ethyl acetate) gave N-[(1R)-1-[3-hydroxy-5-(1-methylpyrazol-4-yl)phenyl]ethyl]-2-methyl-5-(4-methylpiperazin-1-yl)benzamide (217 mg, 48%) as a white foam. ¹H NMR (500 MHz, CDCl₃) δ 7.66 (s, 1H), 7.52 (s, 1H), 7.09-7.04 (m, 1H), 7.00-6.96 (m, 1H), 6.91-6.89 (m, 1H), 6.88-6.84 (m, 1H), 6.82-6.79 (m, 1H), 6.72-6.68 (m, 1H), 6.14-6.07 (m, 1H), 5.28-5.20 (m, 1H), 3.89 (s, 3H), 3.18-3.10 (m, 4H), 2.61-2.53 (m, 4H), 2.35 (s, 3H), 2.30 (s, 3H), 1.55 (d, J=8.1, 3H). LC-MS (Method A): R_{T}=2.50, m/z=434.6 [M+H]⁺.

### Further Examples

The following examples were prepared in a similar manner to N-[(1R)-1-[3-hydroxy-5-(1-methylpyrazol-4-yl)phenyl]ethyl]-2-methyl-5-(4-methylpiperazin-1-yl)benzamide (Example 275).

| Example | Structure | Name and Analytical Data |
|---|---|---|
| 276 | 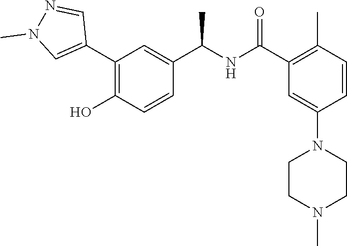 [SMILES: Cc1ccc(N2CCN(C)CC2)cc1C(=O)N[C@H](C)c1ccc(O)c(-c2cnn(C)c2)c1] | N-[(1R)-1-[4-Hydroxy-3-(1- methylpyrazol-4-yl)phenyl]ethyl]-2- methyl-5-(4-methylpiperazin-1- yl)benzamide ¹H NMR (500 MHz, CDCl₃) δ 7.78-7.75 (m, 2H), 7.40 (d, J = 2.1, 1H), 7.12-7.09 (m, 1H), 7.08-7.05 (m, 1H), 6.89-6.85 (m, 2H), 6.84- 6.81 (m, 1H), 6.08-6.03 (m, 1H), 5.32-5.24 (m, 1H), 3.90 (s, 3H), 3.16-3.10 (m, 4H), 2.59-2.53 (m, 4H), 2.34 (s, 3H), 2.31 (s, 3H), 1.61 (d, J = 6.9, 3H). LC-MS (Method A): R_{T} = 2.80, m/z = 434.6 [M + H]⁺. |

### Example 277: N-[(1R)-1-[3-(Cyclopropylmethoxy)-5-(1-methylpyrazol-4-yl)phenyl]ethyl]-2-methyl-5-(4-methylpiperazin-1-yl)benzamide

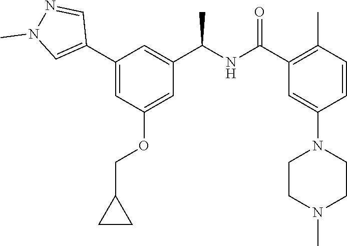 [SMILES: Cc1ccc(N2CCN(C)CC2)cc1C(=O)N[C@H](C)c1cc(OCC2CC2)cc(-c2cnn(C)c2)c1]

Bromomethylcyclopropane (70.06 mg, 518.98 μmol, 50.41 μL) was added to a suspension of N-[(1R)-1-[3-hydroxy-5-(1-methylpyrazol-4-yl)phenyl]ethyl]-2-methyl-5-(4-methylpiperazin-1-yl)benzamide (150 mg, 346 μmol) (Example 275) and potassium carbonate (100 mg, 727 μmol) in DMF (0.45 mL). The tube was sealed and heated to 60° C. for 4 hours. The reaction mixture was allowed to cool to RT and water (20 mL) and ethyl acetate (50 mL) added. The organic phase was washed with brine (10 mL), dried over sodium sulfate and the solvent was removed in vacuo. Purification by FCC (eluting with 0-10% 1M NH₃ in MeOH in DCM) gave, N-[(1R)-1-[3-(cyclopropylmethoxy)-5-(1-methylpyrazol-4-yl)phenyl]ethyl]-2-methyl-5-(4-methylpiperazin-1-yl)benzamide (43 mg, 24%), as a white foam. ¹H NMR (500 MHz, CDCl₃) δ 7.72 (s, 1H), 7.60 (s, 1H), 7.10-7.07 (m, 1H), 7.06-7.04 (m, 1H), 6.94-6.90 (m, 2H), 6.89-6.85 (m, 1H), 6.80-6.76 (m, 1H), 5.97-5.91 (m, 1H), 5.33-5.25 (m, 1H), 3.94 (s, 3H), 3.84 (d, J=6.9, 2H), 3.20-3.12 (m, 4H), 2.60-2.53 (m, 4H), 2.35 (s, 3H), 2.32 (s, 3H), 1.59 (d, J=6.9, 3H), 1.33-1.23 (m, 1H), 0.69-0.62 (m, 2H), 0.39-0.33 (m, 2H). LC-MS (Method A): R_{T}=3.52, m/z=488.7 [M+H]⁺.

### Further Examples

The following examples were prepared in a similar manner to N-[(1R)-1-[3-(cyclopropylmethoxy)-5-(1-methylpyrazol-4-yl)phenyl]ethyl]-2-methyl-5-(4-methylpiperazin-1-yl)benzamide (Example 277), using the required commercially available electrophile.

| Example | Structure | Name and Analytical Data |
|---|---|---|
| 278 | 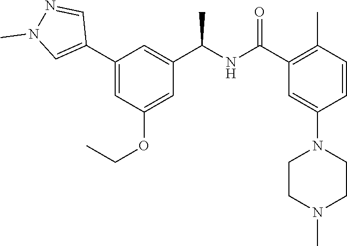 [SMILES: CCOc1cc(-c2cnn(C)c2)cc([C@@H](C)NC(=O)c2cc(N3CCN(C)CC3)ccc2C)c1] | N-[(1R)-1-[3-Ethoxy-5-(1-methylpyrazol- 4-yl)phenyl]ethyl]-2-methyl-5-(4- methylpiperazin-1-yl)benzamide ¹H NMR (500 MHz, CDCl₃) δ 7.73 (s, 1H), 7.60 (s, 1H), 7.10-7.07 (m, 1H), 7.06-7.03 (m, 1H), 6.94-6.90 (m, 2H), 6.89-6.85 (m, 1H), 6.80-6.75 (m, 1H), 5.95 (br d, J = 7.9, 1H), 5.33-5.25 (m, 1H), 4.07 (q, J = 7.0, 2H), 3.93 (s, 3H), 3.19-3.11 (m, 4H), 2.60-2.52 (m, 4H), 2.34 (s, 3H), 2.32 (s, 3H), 1.60 (d, J = 6.9, 3H), 1.43 (t, J = 7.0, 3H). LC-MS (Method A): R_{T} = 3.06, m/z = 462.7 [M + H]⁺. |
| 279 | 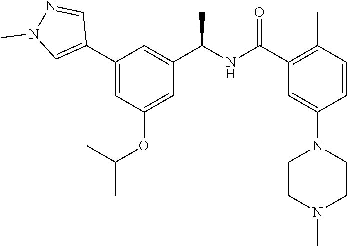 [SMILES: Cc1ccc(N2CCN(C)CC2)cc1C(=O)N[C@H](C)c1cc(OC(C)C)cc(-c2cnn(C)c2)c1] | N-[(1R)-1-[3-Isopropoxy-5-(1- methylpyrazol-4-yl)phenyl]ethyl]-2- methyl-5-(4-methylpiperazin-1- yl)benzamide ¹H NMR (500 MHz, CDCl₃) δ 7.72 (s, 1H), 7.59 (s, 1H), 7.08 (d, J = 8.4, 1H), 7.04 (s, 1H), 6.92 (d, J = 2.6, 1H), 6.90 (s, 1H), 6.89- 6.85 (m, 1H), 6.79-6.76 (m, 1H), 5.94 (br d, J = 7.9, 1H), 5.32-5.25 (m, 1H), 4.64-4.55 (m, 1H), 3.94 (s, 3H), 3.19-3.12 (m, 4H), 2.58-2.52 (m, 4H), 2.35 (s, 3H), 2.31 (s, 3H), 1.60 (d, J = 6.9, 3H), 1.38-1.32 (m, 6H). LC-MS (Method A): R_{T} = 3.37, m/z = 476.7 [M + H]⁺. |
| 280 | 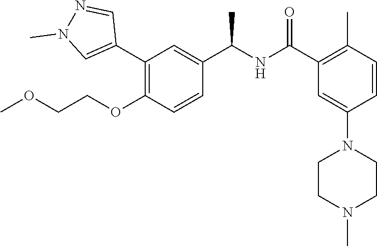 [SMILES: COCCOc1ccc([C@@H](C)NC(=O)c2cc(N3CCN(C)CC3)ccc2C)cc1-c1cnn(C)c1] | N-[(1R)-1-[4-(2-Methoxyethoxy)-3-(1- methylpyrazol-4-yl)phenyl]ethyl]-2- methyl-5-(4-methylpiperazin-1- yl)benzamide ¹H NMR (500 MHz, DMSO-d₆) δ 8.53 (d, J = 8.2, 1H), 8.07 (s, 1H), 7.86 (s, 1H), 7.60 (d, J = 2.0, 1H), 7.14 (dd, J = 2.0, 8.4, 1H), 7.05-6.94 (m, 2H), 6.90-6.84 (m, 1H), 6.80 (d, J = 2.4, 1H), 5.08-5.02 (m, 1H), 4.17-4.08 (m, 2H), 3.84 (s, 3H), 3.77-3.68 (m, 2H), 3.33 (s, 3H), 3.08-3.02 (m, 4H), 2.42-2.37 (m, 4H), 2.18 (s, 3H), 2.13 (s, 3H), 1.39 (d, J = 7.0, 3H). LC-MS (Method A): R_{T} = 3.32, m/z = 492.7 [M + H]⁺. |

### Example 281: N-[(1R)-1-[3-(2-Hydroxyethoxy)-5-(1-methylpyrazol-4-yl)phenyl]ethyl]-2-methyl-5-(4-methylpiperazin-1-yl)benzamide

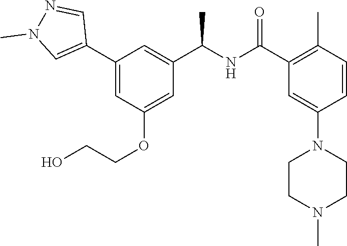 [SMILES: Cc1ccc(N2CCN(C)CC2)cc1C(=O)N[C@H](C)c1cc(OCCO)cc(-c2cnn(C)c2)c1]

Using General Procedure 4 with N-[(1R)-1-[3-[2-[tert-butyl(dimethyl)silyl]oxyethoxy]-5-(1-methylpyrazol-4-yl)phenyl]ethyl]-2-methyl-5-(4-methylpiperazin-1-yl)benzamide (152 mg, 257 μmol)—prepared in a similar manner to N-[(1R)-1-[3-(cyclopropylmethoxy)-5-(1-methylpyrazol-4-yl)phenyl]ethyl]-2-methyl-5-(4-methylpiperazin-1-yl)benzamide (Example 277)—gave N-[(1R)-1-[3-(2-hydroxyethoxy)-5-(1-methylpyrazol-4-yl)phenyl]ethyl]-2-methyl-5-(4-methylpiperazin-1-yl)benzamide (123 mg, 91%) as a white foam. ¹H NMR (500 MHz, CDCl₃) δ 7.73 (s, 1H), 7.60 (s, 1H), 7.11-7.06 (m, 2H), 6.95-6.91 (m, 2H), 6.90-6.86 (m, 1H), 6.83-6.80 (m, 1H), 6.00-5.95 (m, 1H), 5.33-5.25 (m, 1H), 4.15-4.09 (m, 2H), 4.00-3.96 (m, 2H), 3.94 (s, 3H), 3.19-3.12 (m, 4H), 2.58-2.52 (m, 4H), 2.36-2.33 (m, 3H), 2.33-2.31 (m, 3H), 1.60 (d, J=6.9, 3H). LC-MS (Method A): R_{T}=2.55, m/z=478.6 [M+H]⁺.

### Further Examples

The following examples were prepared in a similar manner to N-[(1R)-1-[3-(2-hydroxyethoxy)-5-(1-methylpyrazol-4-yl)phenyl]ethyl]-2-methyl-5-(4-methylpiperazin-1-yl)benzamide (Example 281).

| Example | Structure | Name and Analytical Data |
|---|---|---|
| 282 | 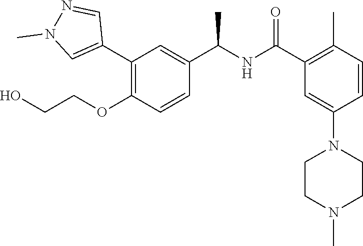 [SMILES: Cc1ccc(N2CCN(C)CC2)cc1C(=O)N[C@H](C)c1ccc(OCCO)c(-c2cnn(C)c2)c1] | N-[(1R)-1-[4-(2-Hydroxyethoxy)-3-(1- methylpyrazol-4-yl)phenyl]ethyl]-2- methyl-5-(4-methylpiperazin-1- yl)benzamide ¹H NMR (500 MHz, CDCl₃) δ 7.88-7.86 (m, 1H), 7.85-7.83 (m, 1H), 7.52-7.48 (m, 1H), 7.23-7.18 (m, 1H), 7.10-7.05 (m, 1H), 6.96- 6.92 (m, 1H), 6.92-6.89 (m, 1H), 6.89-6.83 (m, 1H), 5.97-5.91 (m, 1H), 5.34-5.26 (m, 1H), 4.20-4.15 (m, 2H), 4.04-3.99 (m, 2H), 3.94 (s, 3H), 3.21-3.13 (m, 4H), 2.63-2.55 (m, 4H), 2.36 (s, 3H), 2.31 (s, 3H), 1.61 (d, J = 6.9, 3H). LC-MS (Method A): R_{T} = 2.49, m/z = 478.6 [M + H]⁺. |

### Example 283: N-[(1R)-1-[3-(Difluoromethoxy)-5-(1-methylpyrazol-4-yl)phenyl]ethyl]-2-methyl-5-(4-methylpiperazin-1-yl)benzamide

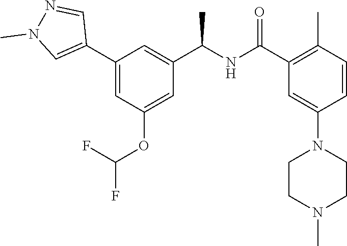 [SMILES: Cc1ccc(N2CCN(C)CC2)cc1C(=O)N[C@H](C)c1cc(OC(F)F)cc(-c2cnn(C)c2)c1]

Under inert atmosphere, cesium carbonate (297.60 mg, 913.40 μmol) was added to a mixture of N-[(1R)-1-[3-hydroxy-5-(1-methylpyrazol-4-yl)phenyl]ethyl]-2-methyl-5-(4-methylpiperazin-1-yl)benzamide (198 mg, 457 μmol) (Example 275) and sodium chlorodifluoroacetate (104 mg, 685 μmol) in DMF (1.5 mL) then heated to 120° C. for 4 hours. The reaction mixture was allowed to cool to RT and water (20 mL) and ethyl acetate (50 mL) added. The organic phase was washed with brine (10 mL), dried over sodium sulfate and the solvent was removed in vacuo. Purification by FCC (eluting with 0-100% MeOH in ethyl acetate gave N-[(1R)-1-[3-(difluoromethoxy)-5-(1-methylpyrazol-4-yl)phenyl]ethyl]-2-methyl-5-(4-methylpiperazin-1-yl)benzamide (90 mg, 36%) as a yellow foam. ¹H NMR (500 MHz, CDCl₃) δ 7.76-7.72 (m, 1H), 7.63 (s, 1H), 7.33 (s, 1H), 7.13-7.06 (m, 2H), 7.00-6.96 (m, 1H), 6.93 (d, J=2.6, 1H), 6.91-6.86 (m, 1H), 6.54 (t, J=74.3, 1H), 6.00-5.94 (m, 1H), 5.36-5.27 (m, 1H), 3.95 (s, 3H), 3.20-3.12 (m, 4H), 2.60-2.52 (m, 4H), 2.35 (s, 3H), 2.31 (s, 3H), 1.59 (d, J=6.9, 3H). LC-MS (Method A): R_{T}=3.33, m/z=484.6 [M+H]⁺.

### Example 284: N-[(1R)-1-[3-[1-(2-Hydroxyethyl)pyrazol-4-yl]-4-methoxy-phenyl]ethyl]-2-methyl-5-(4-methylpiperazin-1-yl)benzamide

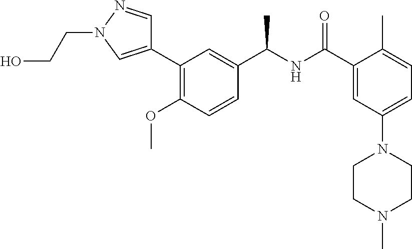 [SMILES: COc1ccc([C@@H](C)NC(=O)c2cc(N3CCN(C)CC3)ccc2C)cc1-c1cnn(CCO)c1]

### Step A: (S)—N-[(1R)-1-[3-[1-[2-[tert-Butyl(dimethyl)silyl]oxyethyl]pyrazol-4-yl]-4-methoxy-phenyl]ethyl]-2-methyl-propane-2-sulfinamide

Using General Procedure 2 with tert-butyl-dimethyl-[2-[4-(4,4,5,5-tetramethyl-1,3,2-dioxaborolan-2-yl)pyrazol-1-yl]ethoxy]silane (1.30 g, 3.69 mmol) and (S)—N-[(1R)-1-(3-bromo-4-methoxy-phenyl)ethyl]-2-methyl-propane-2-sulfinamide (1.23 g, 3.69 mmol)—prepared in a similar manner to (S)—N-[(1R)-1-(5-bromo-2-methoxy-phenyl)ethyl]-2-methyl-propane-2-sulfinamide (Example 239, Step B)—at 60° C. for 90 mins with purification by FCC (eluting with 60-100% diethyl ether in petroleum ether then 100% ethyl acetate) gave (S)—N-[(1R)-1-[3-[1-[2-[tert-butyl(dimethyl)silyl]oxyethyl]pyrazol-4-yl]-4-methoxy-phenyl]ethyl]-2-methyl-propane-2-sulfinamide (1.50 g, 83%) as a yellow gum. LC-MS (Method B): R_{T}=4.41, m/z=478.9 [M−H]⁻.

### Step B: 2-[4-[5-[(1R)-1-Aminoethyl]-2-methoxy-phenyl]pyrazol-1-yl]ethanol Hydrochloride Salt

Using General Procedure 4 with (S)—N-[(1R)-1-[3-[1-[2-[tert-butyl(dimethyl)silyl]oxyethyl]pyrazol-4-yl]-4-methoxy-phenyl]ethyl]-2-methyl-propane-2-sulfinamide (1.50 g, 3.13 mmol) gave 2-[4-[5-[(1R)-1-aminoethyl]-2-methoxy-phenyl]pyrazol-1-yl]ethanol hydrochloride salt (903 mg, 97%) as a white solid. ¹H NMR (500 MHz, DMSO-d₆) δ 8.51 (br s, 3H), 8.19 (s, 1H), 8.01 (s, 1H), 7.91-7.87 (m, 1H), 7.34 (dd, J=2.3, 8.4, 1H), 7.13 (d, J=8.5, 1H), 4.45-4.33 (m, 1H), 4.22 (t, J=5.6, 2H), 3.92 (s, 3H), 3.80 (t, J=5.5, 2H), 3.61 (s, 1H), 1.57 (d, J=6.7, 3H).

### Step C: N-[(1R)-1-[3-[1-(2-Hydroxyethyl)pyrazol-4-yl]-4-methoxy-phenyl]ethyl]-2-methyl-5-(4-methylpiperazin-1-yl)benzamide

Using General Procedure 1 with 2-[4-[5-[(1R)-1-aminoethyl]-2-methoxy-phenyl]pyrazol-1-yl]ethanol hydrochloride salt (105 mg, 402 μmol) and 2-methyl-5-(4-methylpiperazin-1-yl)benzoic acid hydrochloride salt (113 mg, 482 μmol) (Example 21, Step B) with purification by FCC (eluting with 40% MeOH in ethyl acetate) gave N-[(1R)-1-[3-[1-(2-hydroxyethyl)pyrazol-4-yl]-4-methoxy-phenyl]ethyl]-2-methyl-5-(4-methylpiperazin-1-yl)benzamide (37 mg, 20%) as a white foam. ¹H NMR (500 MHz, CDCl₃) δ 7.94-7.90 (m, 2H), 7.51 (d, J=2.4, 1H), 7.22 (dd, J=2.3, 8.4, 1H), 7.08 (d, J=8.5, 1H), 6.94 (d, J=8.5, 1H), 6.91-6.86 (m, 2H), 5.92 (brd, J=7.9, 1H), 5.36-5.25 (m, 1H), 4.31-4.26 (m, 2H), 4.11-3.97 (m, 2H), 3.91 (s, 3H), 3.20-3.07 (m, 4H), 2.63-2.50 (m, 4H), 2.71 (s, 3H), 2.63 (s, 3H), 1.60 (d, J=7.0, 3H). LC-MS (Method B): R_{T}=3.65 m/z=458.9 [M−H]⁻.

### Example 285: 4-[2-Methoxy-5-[(1R)-1-[[2-methyl-5-(4-methylpiperazin-1-yl)benzoyl]amino]ethyl]phenyl]-N,N-dimethyl-thiophene-2-carboxamide

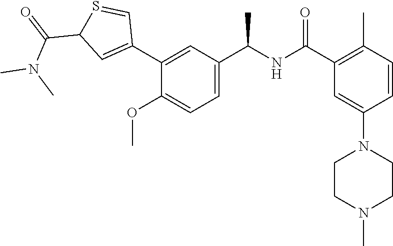

### Step A: Benzyl 4-bromothiophene-2-carboxylate

Benzyl bromide (1.75 mL, 14.7 mmol) was added to a solution of 4-bromo-thiophene-2-carboxylic acid (3.05 g, 14.7 mmol) and potassium carbonate (3.05 g, 22.1 mmol) in DMF (10 mL) and the reaction mixture allowed to stir at RT overnight. Water (100 mL) and petroleum ether (100 mL) was added, and the phases separated. The aqueous phase was extracted with petroleum ether (100 mL) and the combined organic phases washed with brine (120 mL), dried over sodium sulfate and the solvent removed in vacuo. Purification by FCC (eluting with 0-20% ethyl acetate in petroleum ether) gave benzyl 4-bromothiophene-2-carboxylate (2.60 g, 59%) as a colourless oil. ¹H NMR (500 MHz, CDCl₃) δ 7.72 (d, J=1.5, 1H), 7.46 (d, J=1.5, 1H), 7.74-7.35 (m, 5H), 5.34 (s, 2H).

### Step B: Benzyl 4-(4,4,5,5-tetramethyl-1,3,2-dioxaborolan-2-yl)thiophene-2-carboxylate

1,1′-Bis(diphenylphosphino)ferrocenepalladium (II) dichloride (2.11 g, 2.88 mmol) was added to a degassed solution of benzyl 4-bromothiophene-2-carboxylate (8.56 g, 28.8 mmol), potassium pivalate (12.1 g, 86.4 mmol) and bis(pinacolato)diboron (8.78 g, 34.6 mmol) in 1,4-dioxane (80 mL) and the reaction mixture heated at 85° C. overnight. The reaction mixture was allowed to cool to RT and water (150 mL) and ethyl acetate (150 mL) were added and the phases separated. The organic phase was washed with brine (100 mL), dried over sodium sulfate and the solvent removed in vacuo. Purification by FCC (eluting with 0-40% diethyl ether in petroleum ether) gave benzyl 4-(4,4,5,5-tetramethyl-1,3,2-dioxaborolan-2-yl)thiophene-2-carboxylate (3.70 g, 37%) as a yellow oil which solidified on standing to give an off-white solid. ¹H NMR (500 MHz, CDCl₃) δ 8.09 (d, J=1.0, 1H), 8.07 (d, J=1.0, 1H), 7.44-7.42 (m, 2H), 7.40-7.33 (m, 3H), 5.33 (br s, 2H), 1.32 (s, 12H).

### Step C: Benzyl 4-[5-[(1R)-1-[[(S)-tert-butylsulfinyl]amino]ethyl]-2-methoxy-phenyl]thiophene-2-carboxylate

Using General Procedure 2 with (S)—N-[(1R)-1-(3-bromo-4-methoxy-phenyl)ethyl]-2-methyl-propane-2-sulfinamide (980 mg, 2.93 mmol)—prepared in a similar manner to (S)—N-[(1R)-1-(5-bromo-2-methoxy-phenyl)ethyl]-2-methyl-propane-2-sulfinamide (Example 239, Step B)—and benzyl 4-(4,4,5,5-tetramethyl-1,3,2-dioxaborolan-2-yl)thiophene-2-carboxylate (1.01 g, 2.93 mmol) at 85° C. for 3 hours gave benzyl 4-[5-[(1R)-1-[[(S)-tert-butylsulfinyl]amino]ethyl]-2-methoxy-phenyl]thiophene-2-carboxylate (1.22 g, 88%) as pale yellow oil. LC-MS (Method B): R_{T}=4.75, m/z=470.8 [M−H]⁻.

### Step D: Benzyl 4-[5-[(1R)-1-aminoethyl]-2-methoxy-phenyl]thiophene-2-carboxylate

Using General Procedure 4 with benzyl 4-[5-[(1R)-1-[[(S)-tert-butylsulfinyl]amino]ethyl]-2-methoxy-phenyl]thiophene-2-carboxylate (1.22 g, 2.59 mmol gave benzyl 4-[5-[(1R)-1-aminoethyl]-2-methoxy-phenyl]thiophene-2-carboxylate (675 mg, 71%) as a yellow oil. ¹H NMR (500 MHz, DMSO-d₆) δ 8.15 (d, J=1.0, 1H), 7.83 (d, J=1.0, 1H), 7.46-7.43 (m, 3H), 7.39-7.38 (m, 2H), 7.34-7.33 (m, 1H), 7.28 (dd, J=8.5, 2.5, 1H), 6.95 (d, J=8.0, 1H), 5.36 (br s, 2H), 4.13 (q, J=7.0, 1H), 3.86 (s, 3H), 1.63 (br s, 2H), 1.39 (d, J=7.0, 3H).

### Step E: Benzyl 4-[2-methoxy-5-[(1R)-1-[[2-methyl-5-(4-methylpiperazin-1-yl)benzoyl]amino]ethyl]phenyl]thiophene-2-carboxylate

Using General Procedure 1 with benzyl 4-[5-[(1R)-1-aminoethyl]-2-methoxy-phenyl]thiophene-2-carboxylate (141 mg, 384 μmol) and 2-methyl-5-(4-methylpiperazin-1-yl)benzoic acid hydrochloride salt (99 mg, 422 μmol) (Example 21, Step B) gave benzyl 4-[2-methoxy-5-[(1R)-1-[[2-methyl-5-(4-methylpiperazin-1-yl)benzoyl]amino]ethyl]phenyl]thiophene-2-carboxylate (174 mg, 78%) as a white crystalline solid. LC-MS (Method B): R_{T}=4.49, m/z=583.0 [M−H]⁻.

### Step F: 4-[2-Methoxy-5-[(1R)-1-[[2-methyl-5-(4-methylpiperazin-1-yl)benzoyl]amino]ethyl]phenyl]thiophene-2-carboxylic Acid

Using General Procedure 5 with benzyl 4-[2-methoxy-5-[(1R)-1-[[2-methyl-5-(4-methylpiperazin-1-yl)benzoyl]amino]ethyl]phenyl]thiophene-2-carboxylate (174 mg, 298 μmol) and palladium hydroxide, 20% on carbon (30 mg) gave 4-[2-methoxy-5-[(1R)-1-[[2-methyl-5-(4-methylpiperazin-1-yl)benzoyl]amino]ethyl]phenyl]thiophene-2-carboxylic acid (107 mg, 73%) as an off-white solid. LC-MS (Method B): R_{T}=0.50, m/z=494.7 [M+H]⁺.

### Step G: 4-[2-Methoxy-5-[(1R)-1-[[2-methyl-5-(4-methylpiperazin-1-yl)benzoyl]amino]ethyl]phenyl]-N,N-dimethyl-thiophene-2-carboxamide

Using General Procedure 1 with 4-[2-methoxy-5-[(1R)-1-[[2-methyl-5-(4-methylpiperazin-1-yl)benzoyl]amino]ethyl]phenyl]thiophene-2-carboxylic acid (50 mg, 101 μmol) and dimethylamine (2M in THF, 253 μL) gave 4-[2-methoxy-5-[(1R)-1-[[2-methyl-5-(4-methylpiperazin-1-yl)benzoyl]amino]ethyl]phenyl]-N,N-dimethyl-thiophene-2-carboxamide (26 mg, 47%) as a white crystalline solid. ¹H NMR (500 MHz, DMSO-d₆) δ 8.59 (d, J=8.0, 1H), 7.91 (br s, 1H), 7.76 (br s, 1H), 7.59 (d, J=2.0, 1H), 7.33 (dd, J=8.5, 2.0, 1H), 7.09 (d, J=8.5, 1H), 7.06 (d, J=8.0, 1H), 6.90 (dd, J=8.5, 2.5, 1H), 6.83 (d, J=2.5, 1H), 5.12 (quin, J=7.0, 1H), 3.84 (s, 3H), 3.13 (br s, 6H), 3.09-3.08 (m, 4H), 2.43-2.42 (m, 4H), 2.22 (s, 3H), 2.17 (s, 3H), 1.44 (d, J=7.0, 3H). LC-MS (Method B): R_{T}=3.38, m/z=520.0 [M−H]⁻.

### Further Examples

The following examples were prepared in a similar manner to 4-[2-methoxy-5-[(1R)-1-[[2-methyl-5-(4-methylpiperazin-1-yl)benzoyl]amino]ethyl]phenyl]-N,N-dimethyl-thiophene-2-carboxamide (Example 285), using the required commercially available heterocycle in Step A, the required carboxylic acid—prepared in a similar manner to 2-methyl-5-(4-methylpiperazin-1-yl)benzoic acid hydrochloride salt (Example 21, Step B)—in Step E, and the required commercially available amine in Step G.

| Example | Structure | Name and Analytical Data |
|---|---|---|
| 286 | 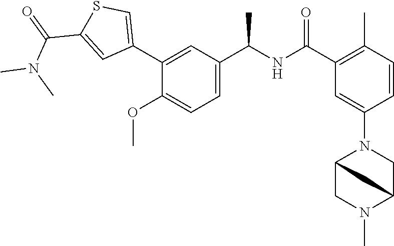 [SMILES: COc1ccc([C@@H](C)NC(=O)c2cc(N3C[C@H]4C[C@@H]3CN4C)ccc2C)cc1-c1csc(C(=O)N(C)C)c1] | 4-[2-Methoxy-5-[(1R)-1-[[2-methyl-5- [(1R,4R)-5-methyl-2,5- diazabicyclo[2.2.1]heptan-2- yl]benzoyl]amino]ethyl]phenyl]-N,N- dimethyl-thiophene-2-carboxamide ¹H NMR (500 MHz, DMSO-d₆) δ 8.57 (d, J = 8.5, 1H), 7.90 (d, J = 1.0, 1H), 7.77 (d, J = 1.0, 1H), 7.60 (d, J = 2.0, 1H), 7.33 (dd, J = 8.5, 2.0, 1H), 7.08 (d, J = 8.5, 1H), 6.98 (d, J = 8.5, 1H), 6.53 (dd, J = 8.0, 2.5, 1H), 6.46 (d, J = 2.5, 1H), 5.12 (quin, J = 7.0, 1H), 4.21 (s, 1H), 3.84 (s, 3H), 3.37 (br s, 1H), 3.32 (s, 6H), 3.26 (dd, J = 9.0, 2.0, 1H), 3.11 (br d, J = 9.0, 1H), 2.70 (dd, J = 9.5, 2.0, 1H), 2.43 (d, J = 9.0, 1H), 2.22 (s, 3H), 2.14 (s, 3H), 1.83 |
| | | (br d, J = 9.0, 1H), 1.72 (br d, J = 9.0, 1H), 1.43 |
| | | (d, J = 7.0, 3H). LC-MS (Method B): R_{T} = |
| | | 3.71, m/z = 532.0 [M − H]⁻. |
| 287 | 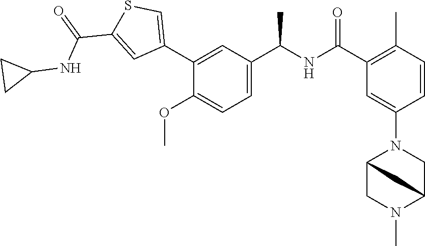 [SMILES: COc1ccc([C@@H](C)NC(=O)c2cc(N3C[C@H]4C[C@@H]3CN4C)ccc2C)cc1-c1csc(C(=O)NC2CC2)c1] | N-Cyclopropyl-4-[2-methoxy-5-[(1R)-1- [[2-methyl-5-[(1R,4R)-5-methyl-2,5- diazabicyclo[2.2.1]heptan-2- yl]benzoyl]amino]ethyl]phenyl]thiophene- 2-carboxamide ¹H NMR (500 MHz, DMSO-d₆) δ 8.58 (d, J = 8.0, 1H), 8.54 (d, J = 3.5, 1H), 8.07 (br s, 1H), 7.90 (br s, 1H), 7.54 (d, J = 2.0, 1H), 7.34 (dd, J = 8.5, 2.0, 1H), 7.09 (d, J = 8.5, 1H), 6.98 (d, J = 8.0, 1H), 6.53 (dd, J = 8.5, 2.5, 1H), 6.45 (d, J = 2.5, 1H), 5.11 (quin, J = 7.0, 1H), 4.21 (br s, 1H), 3.84 (s, 3H), 3.40 (m, 1H), 3.27 (br d, J = 8.5, 1H), 3.11 (br d, J = 9.0, 1H), 2.81- 2.80 (m, 1H), 2.70-2.69 (m, 1H), 2.45-2.44 (m, 1H), 2.24 (br s, 3H), 2.14 (s, 3H), 1.84 (br d, J = 9.0, 1H), 1.72 (br d, J = 9.0, 1H), 1.44 |
| | | (d, J = 7.0, 3H), 0.71-0.70 (m, 2H), 0.59-0.58 |
| | | (m, 2H). LC-MS (Method B): R_{T} = 3.73, m/z = |
| | | 544.0 [M − H]⁻. |
| 288 | 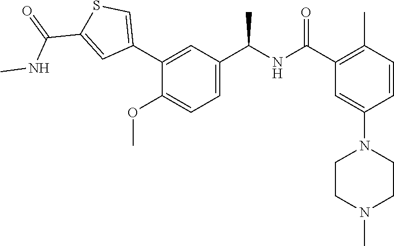 [SMILES: CNC(=O)c1cc(-c2cc([C@@H](C)NC(=O)c3cc(N4CCN(C)CC4)ccc3C)ccc2OC)cs1] | 4-[2-Methoxy-5-[(1R)-1-[[2-methyl-5-(4- methylpiperazin-1- yl)benzoyl]amino]ethyl]phenyl]-N- methyl-thiophene-2-carboxamide ¹H NMR (500 MHz, DMSO-d₆) δ 8.60 (d, J = 8.0, 1H), 8.50 (m, 1H), 8.07 (s, 1H), 7.89 (s, 1H), 7.55 (d, J = 2.0, 1H), 7.34 (dd, J = 8.5, 2.0, 1H), 7.09 (d, J = 8.5, 1H), 7.04 (d, J = 8.5, 1H), 6.90 (dd, J = 8.5, 2.5, 1H), 6.83 (d, J = 2.5, 1H), 5.12 (quin, J = 7.0, 1H), 3.84 (s, 3H), 3.70 (m, 4H), 2.79 (d, J = 4.5, 3H), 2.44-2.43 (m, 4H), 2.22 (s, 3H), 2.17 (s, 3H), 1.45 (d, J = 7.0, 3H). LC-MS (Method B): R_{T} = 3.31, m/z = 505.9 [M − H]⁻. |
| 289 | 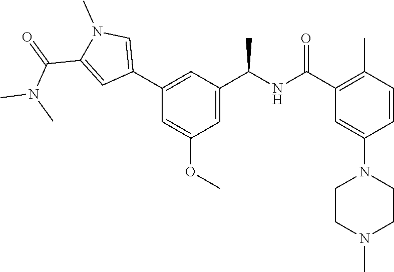 [SMILES: COc1cc(-c2cc(C(=O)N(C)C)n(C)c2)cc([C@@H](C)NC(=O)c2cc(N3CCN(C)CC3)ccc2C)c1] | 4-[3-Methoxy-5-[(1R)-1-[[2-methyl-5-(4- methylpiperazin-1- yl)benzoyl]amino]ethyl]phenyl]-N,N,1- trimethyl-pyrrole-2-carboxamide ¹H NMR (500 MHz, DMSO-d₆) δ 8.60 (d, J = 8.5, 1H), 7.37 (br s, 1H), 7.17 (br s, 1H), 7.07 (d, J = 8.5, 1H), 6.97 (br s, 1H), 6.91 (dd, J = 8.5, 2.0, 1H), 6.85 (d, J = 2.0, 1H), 6.77-6.76 (m, 2H), 5.09 (quin, J = 7.0, 1H), 3.78 (s, 3H), 3.70 (s, 3H), 3.10-3.09 (m, 10H), 2.44-2.43 (m, 4H), 2.22 (s, 3H), 2.19 (s, 3H), 1.43 (d, J = 7.0, 3H). LC-MS (Method B): R_{T} = 3.33, m/z = 516.9 [M − H]⁻. |
| 290 | 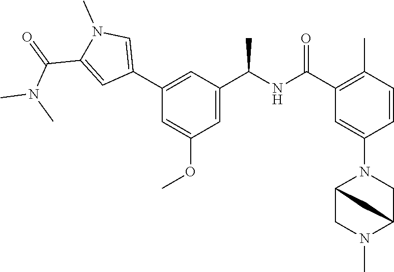 [SMILES: COc1cc(-c2cc(C(=O)N(C)C)n(C)c2)cc([C@@H](C)NC(=O)c2cc(N3C[C@H]4C[C@@H]3CN4C)ccc2C)c1] | 4-[3-Methoxy-5-[(1R)-1-[[2-methyl-5- [(1R,4R)-5-methyl-2,5- diazabicyclo[2.2.1]heptan-2- yl]benzoyl]amino]ethyl]phenyl]-N,N,1- trimethyl-pyrrole-2-carboxamide ¹H NMR (500 MHz, DMSO-d₆) δ 8.57 (d, J = 8.0, 1H), 7.36 (d, J = 1.5, 1H), 7.18 (br s, 1H), 6.99 (d, J = 8.0, 1H), 6.97-6.96 (m, 1H), 6.77-6.76 (m, 2H), 6.55 (dd, J = 8.0, 2.5, 1H), 6.49 (d, J = 2.5, 1H), 5.08 (quin, J = 7.0, 1H), 4.24 (br s, 1H), 3.78 (s, 3H), 3.70 (s, 3H), 3.38 (br s, 1H), 3.29 (br dd, J = 9.0, 2.0, 1H), 3.14 (br d, J = 9.0, 1H), 3.08 (br s, 6H), 2.72 (dd, J = 9.5, 1.5, 1H), 2.45 (d, J = 9.5, 1H), 2.23 (s, 3H), 2.16 (s, 3H), 1.84 (br d, J = 9.0, 1H), 1.73 (br d, J = 9.0, 1H), 1.43 (d, J = 7.0, 3H). LC-MS (Method B): R_{T} = 3.67, m/z = 529.0 [M − H]⁻. |

### Example 291: N-[(1R)-1-(3-Hydroxy-4-methoxy-phenyl)ethyl]-2-methyl-5-(4-methylpiperazin-1-yl)benzamide

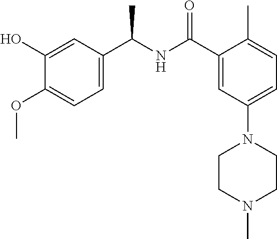 [SMILES: COc1ccc([C@@H](C)NC(=O)c2cc(N3CCN(C)CC3)ccc2C)cc1O]

### Step A: (NE)-N-[(3-Hydroxy-4-methoxy-phenyl)methylene]-2-methyl-propane-2-sulfinamide

A mixture of 3-hydroxy-4-methoxy-benzaldehyde (1.00 g, 6.57 mmol), (S)-2-methylpropane-2-sulfinamide (797 mg, 6.57 mmol), pyridinium p-toluenesulfonate (83 mg, 329 μmol), MgSO₄ (1.58 g, 13.2 mmol) and copper (II) sulfate (2.10 g, 13.2 mmol) in chloroform (20 mL) was heated at reflux overnight. The reaction mixture was allowed to cool then filtered through a pad of Celite. The solid was filtered off and then washed alternately with DCM (2×30 mL) and water (2×20 mL). The filtrate was dried (Na₂SO₄), filtered and concentrated in vacuo to leave the crude product. Purification was by trituration with 50% petroleum ether in DCM, affording (NE)-N-[(3-hydroxy-4-methoxy-phenyl)methylene]-2-methyl-propane-2-sulfinamide (1.32 g, 79%) as a colourless solid. LC-MS (Method B): R_{T}=3.04, m/z=254.5 [M−H]⁻.

### Step B: (NE)-N-[[3-[tert-Butyl(dimethyl)silyl]oxy-4-methoxy-phenyl]methylene]-2-methyl-propane-2-sulfinamide

(NE)-N-[(3-Hydroxy-4-methoxy-phenyl)methylene]-2-methyl-propane-2-sulfinamide (1.32 g, 5.17 mmol) was dissolved into DCM (50 mL), then tert-butyldimethylsilyl chloride (1.17 g, 7.75 mmol) and imidazole (1.06 g, 15.5 mmol) were added. Mixture was stirred overnight at RT, then quenched with sat aq. NH₄Cl. Organic layer was then separated, dried (Na₂SO₄) and concentrated in vacuo to afford (NE)-N-[[3-[tert-butyl(dimethyl)silyl]oxy-4-methoxy-phenyl]methylene]-2-methyl-propane-2-sulfinamide (1.90 g, 99%) as a pale yellow solid. LC-MS (Method A): R_{T}=4.93, m/z=370.6 [M+H]⁺.

### Step C: N-[(1R)-1-[3-[tert-Butyl(dimethyl)silyl]oxy-4-methoxy-phenyl]ethyl]-2-methyl-propane-2-sulfinamide

(NE)-N-[[3-[tert-Butyl(dimethyl)silyl]oxy-4-methoxy-phenyl]methylene]-2-methyl-propane-2-sulfinamide (1.90 g, 5.14 mmol) was dissolved into THF (50 mL), then cooled to −30° C. under nitrogen. Methylmagnesium bromide solution (3M, 5.14 mL) was then added dropwise. Stirring was then continued at −30° C. for 1 hour, then the temperature was allowed to rise slowly over 3 hours. Mixture was then quenched at 0° C. using NH₄Cl sat. aq., then the mixture allowed to warm to RT. Product was then extracted into ethyl acetate (80 mL), and the organic layer was then dried (Na₂SO₄), filtered and concentrated in vacuo to afford crude material. Crude was purified by FCC (eluting with 0-100% ethyl acetate in petroleum ether) to afford N-[(1R)-1-[3-[tert-butyl(dimethyl)silyl]oxy-4-methoxy-phenyl]ethyl]-2-methyl-propane-2-sulfinamide (850 mg, 43%). LC-MS (Method A): R_{T}=4.93, m/z=370.6 [M+H]⁺.

### Step D: (1R)-1-[3-[tert-Butyl(dimethyl)silyl]oxy-4-methoxy-phenyl]ethanamine

Using General Procedure 4 with N-[(1R)-1-[3-[tert-butyl(dimethyl)silyl]oxy-4-methoxy-phenyl]ethyl]-2-methyl-propane-2-sulfinamide (850 mg, 2.20 mmol) gave a mixture of desired product plus desilylated by-product. Resubjecting the crude material to Step B afforded (1R)-1-[3-[tert-butyl(dimethyl)silyl]oxy-4-methoxy-phenyl]ethanamine (1.00 g, 95%) as a yellow gum which was used directly in Step E.

### Step E: N-[(1R)-1-[3-[tert-Butyl(dimethyl)silyl]oxy-4-methoxy-phenyl]ethyl]-2-methyl-5-(4-methylpiperazin-1-yl)benzamide

Using General Procedure 1 with (1R)-1-[3-[tert-butyl(dimethyl)silyl]oxy-4-methoxy-phenyl]ethanamine (1.00 g, 3.55 mmol) and 2-methyl-5-(4-methylpiperazin-1-yl)benzoic acid hydrochloride salt (916 mg, 3.91 mmol) (Example 10, Step B) gave N-[(1R)-1-[3-[tert-butyl(dimethyl)silyl]oxy-4-methoxy-phenyl]ethyl]-2-methyl-5-(4-methylpiperazin-1-yl)benzamide (950 mg, 54%). LC-MS (Method A): R_{T}=4.24, m/z=498.8 [M+H]⁺.

### Step F: N-[(1R)-1-(3-Hydroxy-4-methoxy-phenyl)ethyl]-2-methyl-5-(4-methylpiperazin-1-yl)benzamide

Using General Procedure 4 with N-[(1R)-1-[3-[tert-butyl(dimethyl)silyl]oxy-4-methoxy-phenyl]ethyl]-2-methyl-5-(4-methylpiperazin-1-yl)benzamide (950 mg, 1.91 mmol) gave N-[(1R)-1-(3-hydroxy-4-methoxy-phenyl)ethyl]-2-methyl-5-(4-methylpiperazin-1-yl)benzamide (450 mg, 61%) as a colourless solid. ¹H NMR (500 MHz, DMSO-d₆) δ 8.86 (s, 1H), 8.53-8.46 (m, 1H), 7.08-7.02 (m, 1H), 6.93-6.80 (m, 4H), 6.78-6.73 (m, 1H), 5.05-4.91 (m, 1H), 3.74 (s, 3H), 3.15-3.05 (m, 4H), 2.47-2.42 (m, 4H), 2.22 (s, 3H), 2.17 (s, 3H), 1.37 (d, J=7.0, 3H). LC-MS (Method A): R_{T}=2.78, m/z=384.7 [M+H]⁺.

### Example 292: N-[(1R)-1-[4-Methoxy-3-(2-morpholinoethoxy)phenyl]ethyl]-2-methyl-5-(4-methylpiperazin-1-yl)benzamide

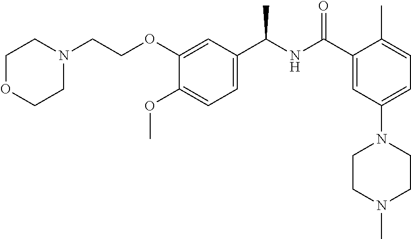 [SMILES: COc1ccc([C@@H](C)NC(=O)c2cc(N3CCN(C)CC3)ccc2C)cc1OCCN1CCOCC1]

N-[(1R)-1-(3-Hydroxy-4-methoxy-phenyl)ethyl]-2-methyl-5-(4-methylpiperazin-1-yl)benzamide (70 mg, 182 μmol) (Example 291), potassium carbonate (53 mg, 383 μmol) and 4-(2-chloroethyl)morpholine (37 mg, 200 μmol) were added to acetonitrile (5 mL) and heated to reflux for 3 hours. The reaction was quenched with water (60 mL), extracted with DCM (50 mL), dried (MgSO₄), filtered and solvent removed in vacuo to afford a yellow gum. Purification by FCC (eluting with 0-10% 7N NH₃/MeOH in ethyl acetate) afforded a gum which was triturated with diethyl ether to afford a solid which was filtered to afford N-[(1R)-1-[4-methoxy-3-(2-morpholinoethoxy)phenyl]ethyl]-2-methyl-5-(4-methylpiperazin-1-yl)benzamide (45 mg, 50%) as a white solid. ¹H NMR (500 MHz, CDCl₃) δ 7.26 (s, 1H), 7.08 (d, J=8.2, 1H), 6.97-6.82 (m, 4H), 5.86 (br d, J=7.9, 1H), 5.26 (quin, J=7.2, 1H), 4.15 (t, J=6.0, 2H), 3.85 (s, 3H), 3.81-3.65 (m, 4H), 3.22-3.10 (m, 4H), 2.84 (t, J=6.1, 2H), 2.57 (td, J=4.5, 9.5, 8H), 2.35 (s, 3H), 2.32-2.29 (m, 3H), 1.57 (d, J=7.0, 3H). LC-MS (Method B): R_{T}=3.26 m/z=496.1 [M−H]⁻.

### Further Examples

The following examples were prepared in a similar manner to N-[(1R)-1-[4-methoxy-3-(2-morpholinoethoxy)phenyl]ethyl]-2-methyl-5-(4-methylpiperazin-1-yl)benzamide (Example 292), using the intermediate Example stated.

| Example | Structure | Name and Analytical Data |
|---|---|---|
| 293 (Example 305) | 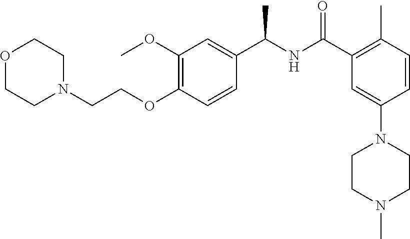 [SMILES: COc1cc([C@@H](C)NC(=O)c2cc(N3CCN(C)CC3)ccc2C)ccc1OCCN1CCOCC1] | N-[(1R)-1-[3-Methoxy-4-(2- morpholinoethoxy)phenyl]ethyl]-2- methyl-5-(4-methylpiperazin-1- yl)benzamide ¹H NMR (500 MHz, CDCl₃) δ 7.11-7.04 (m, 1H), 6.94-6.91 (m, 2H), 6.90-6.84 (m, 3H), 5.88 (br d, J = 8.1, 1H), 5.30-5.22 (m, 1H), 4.15 (t, J = 6.1, 2H), 3.86 (s, 3H), 3.76-3.70 (m, 4H), 3.19-3.11 (m, 4H), 2.84 (t, J = 6.0, 2H), 2.61-2.57 (m, 4H), 2.56-2.52 (m, 4H), 2.35 (s, 3H), 2.30 (s, 3H), 1.58 (d, J = 6.9, 3H). LC-MS (Method A): R_{T} = 2.42, m/z = 497.8 [M + H]⁺. |

### Example 294: N-[(1R)-1-[3-(2-Hydroxyethoxy)-4-methoxy-phenyl]ethyl]-2-methyl-5-(4-methylpiperazin-1-yl)benzamide

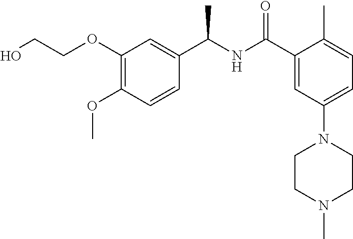 [SMILES: COc1ccc([C@@H](C)NC(=O)c2cc(N3CCN(C)CC3)ccc2C)cc1OCCO]

### Step A: N-[(1R)-1-[3-[2-[tert-Butyl(dimethyl)silyl]oxyethoxy]-4-methoxy-phenyl]ethyl]-2-methyl-5-(4-methylpiperazin-1-yl)benzamide

A solution of N-[(1R)-1-(3-hydroxy-4-methoxy-phenyl)ethyl]-2-methyl-5-(4-methylpiperazin-1-yl)benzamide (60 mg, 156 μmol) (Example 291), 2-bromoethoxy-tert-butyldimethylsilane (45 mg, 188 μmol) and potassium carbonate (45 mg, 329 μmol) in DMF (10 mL) was heated to 50° C. overnight. The reaction mixture was diluted with water (70 mL), then extracted with ethyl acetate (70 mL). The organic phase was washed with brine (60 mL), dried (Na₂SO₄) and the solvent removed in vacuo. Purification by FCC (eluting with 0-50% MeOH in DCM) gave N-[(1R)-1-[3-[2-[tert-butyl(dimethyl)silyl]oxyethoxy]-4-methoxy-phenyl]ethyl]-2-methyl-5-(4-methylpiperazin-1-yl)benzamide (18 mg, 21%). LC-MS (Method B): R_{T}=4.51, m/z=541.1 [M−H]⁻.

### Step B: N-[(1R)-1-[3-(2-Hydroxyethoxy)-4-methoxy-phenyl]ethyl]-2-methyl-5-(4-methylpiperazin-1-yl)benzamide

Using General Procedure 4 with N-[(1R)-1-[3-[2-[tert-butyl(dimethyl)silyl]oxyethoxy]-4-methoxy-phenyl]ethyl]-2-methyl-5-(4-methylpiperazin-1-yl)benzamide (18 mg, 33 μmol) gave N-[(1R)-1-[3-(2-hydroxyethoxy)-4-methoxy-phenyl]ethyl]-2-methyl-5-(4-methylpiperazin-1-yl)benzamide (13 mg, 87%) as a pale orange solid. ¹H NMR (500 MHz, DMSO-d₆) δ 8.54 (d, J=8.0, 1H), 7.07-7.04 (m, 2H), 6.93-6.87 (m, 3H), 6.83 (d, J=3.0, 1H), 5.05 (quin, J=7.0, 1H), 4.84 (t, J=5.5, 1H), 3.96 (td, J=5.5, 2.0, 2H), 3.74 (s, 3H), 3.72 (m, 2H), 3.10 (m, 4H), 2.45 (m, 4H), 2.22 (s, 3H), 2.17 (s, 3H), 1.39 (d, J=7.0, 3H). LC-MS (Method B): R_{T}=3.25, m/z=426.9 [M−H]⁻.

### Example 295: N-[(1R)-1-[3-(2-Aminoethoxy)-4-methoxy-phenyl]ethyl]-2-methyl-5-(4-methylpiperazin-1-yl)benzamide Dihydrochloride Salt

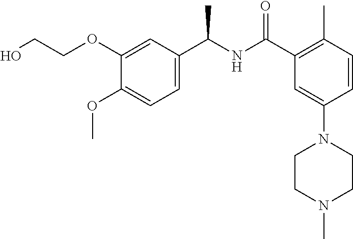 [SMILES: COc1ccc([C@@H](C)NC(=O)c2cc(N3CCN(C)CC3)ccc2C)cc1OCCO]

### Step A: tert-Butyl N-[2-[2-methoxy-5-[(1R)-1-[[2-methyl-5-(4-methylpiperazin-1-yl)benzoyl]amino]ethyl]phenoxy]ethyl]carbamate

N-[(1R)-1-(3-Hydroxy-4-methoxy-phenyl)ethyl]-2-methyl-5-(4-methylpiperazin-1-yl)benzamide (59 mg, 154 μmol) (Example 291) was dissolved in MeCN (10 mL), then potassium carbonate (64 mg, 462 μmol) was added. The mixture was then stirred for 20 mins, then N-boc-2-chloroethylamine (30 mg, 169 μmol) was added. The reaction was then heated to reflux overnight. Reaction was then cooled to RT and partitioned between ethyl acetate and water. Organic layer was dried (Na₂SO₄), filtered and concentrated in vacuo to afford tert-butyl N-[2-[2-methoxy-5-[(1R)-1-[[2-methyl-5-(4-methylpiperazin-1-yl)benzoyl]amino]ethyl]phenoxy]ethyl]carbamate (37 mg, 46%) as a yellow solid. LC-MS (Method A): R_{T}=3.62, m/z=527.9 [M+H]⁺.

### Step B: N-[(1R)-1-[3-(2-Aminoethoxy)-4-methoxy-phenyl]ethyl]-2-methyl-5-(4-methylpiperazin-1-yl)benzamide Dihydrochloride Salt

Using General Procedure 4 with tert-butyl N-[2-[2-methoxy-5-[(1R)-1-[[2-methyl-5-(4-methylpiperazin-1-yl)benzoyl]amino]ethyl]phenoxy]ethyl]carbamate (37 mg, 70 μmol) gave N-[(1R)-1-[3-(2-aminoethoxy)-4-methoxy-phenyl]ethyl]-2-methyl-5-(4-methylpiperazin-1-yl)benzamide dihydrochloride salt (32 mg, 82%) as a pale yellow solid. ¹H NMR (500 MHz, DMSO-d₆) δ 11.31-11.00 (m, 1H), 8.77-8.70 (m, 1H), 8.64 (d, J=8.4, 1H), 8.25 (br s, 4H), 7.18-7.06 (m, 2H), 7.02-6.87 (m, 4H), 5.14-5.02 (m, 1H), 4.32-4.25 (m, 1H), 4.22-4.13 (m, 2H), 3.55-3.36 (m, 4H), 3.13 (br s, 7H), 2.80 (d, J=4.4, 3H), 2.19 (s, 3H), 1.47-1.36 (m, 3H). LC-MS (Method A): R_{T}=1.87, m/z=427.8 [M+H]⁺.

### Example 296: N-[(1R)-1-[3-(Cyclopropylmethoxy)-4-methoxy-phenyl]ethyl]-2-methyl-5-(4-methylpiperazin-1-yl)benzamide

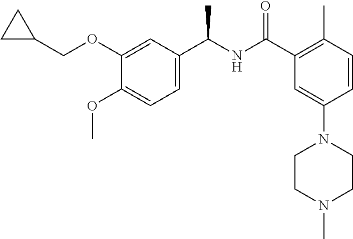 [SMILES: COc1ccc([C@@H](C)NC(=O)c2cc(N3CCN(C)CC3)ccc2C)cc1OCC1CC1]

N-[(1R)-1-(3-Hydroxy-4-methoxy-phenyl)ethyl]-2-methyl-5-(4-methylpiperazin-1-yl)benzamide (122 mg, 318 μmol) (Example 291), potassium carbonate (75 mg, 541 μmol) and (bromomethyl)cyclopropane (47 mg, 350 μmol) were added to DMF (10 mL) and heated to 70° C. overnight. The reaction was quenched with water (100 ml), extracted with ethyl acetate (2×75 ml), dried and solvent evaporated to afford a clear liquid. Purification by FCC (eluting with 0-50% MeOH in ethyl acetate) gave N-[(1R)-1-[3-(cyclopropylmethoxy)-4-methoxy-phenyl]ethyl]-2-methyl-5-(4-methylpiperazin-1-yl)benzamide (14 mg, 9%) as an off-white crystalline solid. ¹H NMR (500 MHz, DMSO-d₆) δ 8.53 (d, J=8.5, 1H), 7.06 (d, J=8.5, 1H), 7.60-7.59 (m, 1H), 6.91-6.86 (m, 3H), 6.82 (d, J=2.5, 1H), 5.04 (quin, J=7.0, 1H), 3.79 (d, J=7.0, 1H), 3.75 (s, 3H), 3.10-3.09 (m, 4H), 2.45-2.44 (m, 4H), 2.22 (s, 3H), 2.16 (s, 3H), 1.38 (d, J=7.0, 3H), 1.24-1.23 (m, 1H), 0.56-0.55 (m, 2H), 0.30-0.29 (m, 2H). LC-MS (Method B): R_{T}=3.47, m/z=436.8 [M−H]⁻.

### Example 297: N-[(1R)-1-[4-(Cyclopropylmethoxy)-3-methoxy-phenyl]ethyl]-2-methyl-5-[(1R,4R)-5-methyl-2,5-diazabicyclo[2.2.1]heptan-2-yl]benzamide

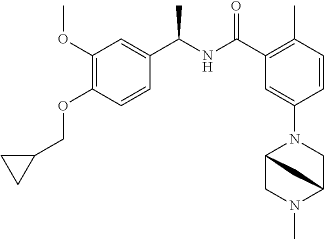 [SMILES: COc1cc([C@@H](C)NC(=O)c2cc(N3C[C@H]4C[C@@H]3CN4C)ccc2C)ccc1OCC1CC1]

### Step A: 4-(Cyclopropylmethoxy)-3-methoxy-benzaldehyde

4-Hydroxy-3-methoxy-benzaldehyde (5.00 g, 32.9 mmol), potassium carbonate (9.08 g, 65.7 mmol) and (bromomethyl)cyclopropane (3.51 mL, 36.15 mmol) were added to DMF (50 mL) and heated to 70° C. overnight. The reaction was cooled to RT and water (200 ml) added and the resulting solid filtered and allowed to dry under vacuum which gave 4-(cyclopropylmethoxy)-3-methoxy-benzaldehyde (6.27 g, 93%) as a white crystalline solid. ¹H NMR (500 MHz, CDCl₃) δ 9.84 (s, 1H), 7.43-7.41 (m, 2H), 6.95 (d, J=8.0, 1H), 3.94 (m, 5H), 1.37 (m, 1H), 0.69 (m, 2H), 0.39 (m, 2H).

### Step B: (NE,S)—N-[[4-(Cyclopropylmethoxy)-3-methoxy-phenyl]methylene]-2-methyl-propane-2-sulfinamide

(S)-(−)-2-Methylpropane-2-sulphonamide (3.68 g, 30.4 mmol) was added to a solution of 4-(cyclopropylmethoxy)-3-methoxy-benzaldehyde (6.27 g, 30.4 mmol) and cesium carbonate (9.91 g, 30.4 mmol) in DCM (300 mL) and the reaction mixture heated to reflux over the weekend. The reaction mixture was allowed to cool to RT. Water (120 mL) and DCM (120 mL) were added and the phases separated. The aqueous phase was extracted with DCM (70 mL). The combined organic phases were washed with brine (150 mL), dried over sodium sulfate and the solvent removed in vacuo which gave (NE,S)—N-[[4-(cyclopropylmethoxy)-3-methoxy-phenyl]methylene]-2-methyl-propane-2-sulfinamide (3.02 g, 32%) as a yellow oil. ¹H NMR (500 MHz, CDCl₃) δ 8.40 (s, 1H), 7.37 (d, J=1.5, 1H), 7.27 (dd, J=8.0, 2.0, 1H), 6.84 (d, J=8.0, 1H), 3.88-3.85 (m, 5H), 1.29 (m, 1H), 1.19 (s, 9H), 0.61 (m, 2H), 0.32 (m, 2H).

### Step C: (S)—N-[(1R)-1-[4-(Cyclopropylmethoxy)-3-methoxy-phenyl]ethyl]-2-methyl-propane-2-sulfinamide

Methylmagnesium bromide solution (3.0 M, 14.18 mL) was added to a solution of (NE,S)—N-[[4-(cyclopropylmethoxy)-3-methoxy-phenyl]methylene]-2-methyl-propane-2-sulfinamide (9.40 g, 30.4 mmol) in DCM at 0° C. and the reaction mixture was allowed to warm to RT and stirred overnight. A mixture of water and saturated aqueous ammonium chloride (1:1, 150 mL) was added carefully followed by DCM (50 mL). The phases were separated, and the organic phase washed with brine (150 mL), dried over sodium sulfate and the solvent removed in vacuo. Purification by FCC (eluting with 0-100% ethyl acetate in diethyl ether) gave (S)—N-[(1R)-1-[4-(cyclopropylmethoxy)-3-methoxy-phenyl]ethyl]-2-methyl-propane-2-sulfinamide (3.20 g, 32%) as a yellow oil. ¹H NMR (500 MHz, CDCl₃) 6.86-6.80 (m, 3H), 4.45 (qd, J=6.5, 3.5, 1H), 3.79 (s, 3H), 3.77 (d, J=7.0, 2H), 3.22 (d, J=2.5, 1H), 1.45 (d, J=6.5, 3H), 1.26 (m, 1H), 1.13 (s, 9H), 0.56 (m, 2H), 0.28 (m, 2H). LC-MS (Method B): R_{T}=3.25, m/z=324.6 [M−H]⁻.

### Step D: (1R)-1-[4-(Cyclopropylmethoxy)-3-methoxy-phenyl]ethanamine Hydrochloride Salt

Using General Procedure 4 with (S)—N-[(1R)-1-[4-(cyclopropylmethoxy)-3-methoxy-phenyl]ethyl]-2-methyl-propane-2-sulfinamide (3.02 g, 9.28 mmol) gave (1R)-1-[4-(cyclopropylmethoxy)-3-methoxy-phenyl]ethanamine hydrochloride (2.20 g, 92%) as a white solid. ¹H NMR (500 MHz, DMSO-d₆) 8.37 (br s, 3H), 7.20 (s, 1H), 6.97-6.93 (m, 2H), 4.31 (m, 1H), 3.80 (s, 3H), 3.78 (m, 2H), 1.49 (d, J=7.0, 3H), 1.20 (m, 1H), 0.58 (m, 2H), 0.30 (m, 2H).

### Step E: N-[(1R)-1-[4-(Cyclopropylmethoxy)-3-methoxy-phenyl]ethyl]-2-methyl-5-[(1R,4R)-5-methyl-2,5-diazabicyclo[2.2.1]heptan-2-yl]benzamide

Using General Procedure 1 with 2-methyl-5-[(1R,4R)-5-methyl-2,5-diazabicyclo[2.2.1]heptan-2-yl]benzoic acid (122 mg, 497 μmol)—prepared in a similar manner to 2-methyl-5-(4-methylpiperazin-1-yl)benzoic acid hydrochloride salt (Example 21, Step B)—and (1R)-1-[4-(cyclopropylmethoxy)-3-methoxy-phenyl]ethanamine hydrochloride salt (100 mg, 452 μmol) with purification by FCC (eluting with 0-100% MeOH in ethyl acetate) gave N-[(1R)-1-[4-(cyclopropylmethoxy)-3-methoxy-phenyl]ethyl]-2-methyl-5-[(1R,4R)-5-methyl-2,5-diazabicyclo[2.2.1]heptan-2-yl]benzamide (89 mg, 42%) as an off-white solid. ¹H NMR (500 MHz, DMSO-d₆) δ 8.52 (d, J=8.0, 1H), 7.03 (br s, 1H), 6.98 (d, J=8.0, 1H), 6.86 (br s, 2H), 6.54 (dd, J=8.0, 2.5, 1H), 6.46 (d, J=2.5, 1H), 5.06 (quin, J=7.0, 1H), 4.24 (br s, 1H), 3.77 (s, 3H), 3.75 (m, 2H), 3.39 (br s, 1H), 3.29 (d, J=9.0, 2.0, 1H), 3.13 (d, J=9.0, 1H), 2.75 (m, 1H), 2.45 (br d, J=9.0, 1H), 2.24 (s, 3H), 2.14 (s, 3H), 1.85 (br d, J=9.0, 1H), 1.73 (br d, J=9.0, 1H), 1.39 (d, J=7.0, 3H), 1.21 (m, 1H), 0.56 (m, 2H), 0.30 (q, J=4.5, 2H). LC-MS (Method B): R_{T}=3.85, m/z=448.8 [M−H]⁻.

### Example 298: N-[(1R)-1-[4-(Cyclopropylmethoxy)-3-methoxy-phenyl]ethyl]-5-[(1R,5S)-3,8-diazabicyclo[3.2.1]octan-3-yl]-2-methyl-benzamide

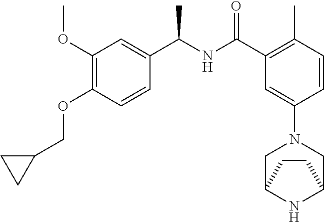 [SMILES: COc1cc([C@@H](C)NC(=O)c2cc(N3C[C@H]4CC[C@@H](C3)N4)ccc2C)ccc1OCC1CC1]

Using General Procedure 4 with tert-butyl (1R,5S)-3-[3-[[(1R)-1-[4-(cyclopropylmethoxy)-3-methoxy-phenyl]ethyl]carbamoyl]-4-methyl-phenyl]-3,8-diazabicyclo[3.2.1]octane-8-carboxylate (158 mg, 287 μmol)—prepared in a similar manner to N-[(1R)-1-[4-(cyclopropylmethoxy)-3-methoxy-phenyl]ethyl]-2-methyl-5-[(1R,4R)-5-methyl-2,5-diazabicyclo[2.2.1]heptan-2-yl]benzamide (Example 296)—gave N-[(1R)-1-[4-(cyclopropylmethoxy)-3-methoxy-phenyl]ethyl]-5-[(1R,5S)-3,8-diazabicyclo[3.2.1]octan-3-yl]-2-methyl-benzamide (71 mg, 49%) as a pale yellow solid. ¹H NMR (500 MHz, DMSO-d₆) δ 8.52 (d, J=8.5, 1H), 7.01 (m, 2H), 6.85 (m, 2H), 6.75 (dd, J=8.5, 2.5, 1H), 6.68 (d, J=2.5, 1H), 5.05 (quin, J=7.0, 1H), 3.77 (s, 3H), 3.76 (m, 2H), 3.49 (br s, 2H), 3.36 (m, 2H), 2.70 (br d, J=10.5, 2H), 2.14 (s, 3H), 1.67 (br s, 4H), 1.39 (d, J=7.0, 3H), 1.21 (m, 1H), 0.55 (m, 2H), 0.29 (m, 2H). LC-MS: R_{T}=3.98, m/z=448.9 [M−H]⁻.

### Example 299: N-[(1R)-1-(3-Bromo-4-methoxy-phenyl)ethyl]-2-methyl-5-(1-methyl-4-piperidyl)benzamide

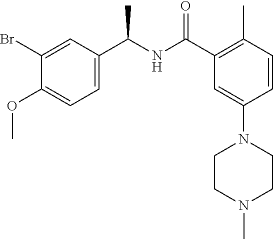 [SMILES: COc1ccc([C@@H](C)NC(=O)c2cc(N3CCN(C)CC3)ccc2C)cc1Br]

### Step A: 2-Methyl-5-(1-methyl-3,6-dihydro-2H-pyridin-4-yl)benzoate

Using General Procedure 2 with 1-methyl-4-(4,4,5,5-tetramethyl-1,3,2-dioxaborolan-2-yl)-3,6-dihydro-2H-pyridine (4.49 g, 20 mmol) and benzyl 5-iodo-2-methyl-benzoate (5.90 g, 17 mmol) at 60° C. for 2 hours gave benzyl 2-methyl-5-(1-methyl-3,6-dihydro-2H-pyridin-4-yl)benzoate (4.26 g, 79%) as a yellow oil. LC-MS (Method B): R_{T}=4.59, m/z=322.6 [M+H]⁺.

### Step B: 2-Methyl-5-(1-methyl-4-piperidyl)benzoic Acid

Using General Procedure 5 with palladium hydroxide, 20% on carbon (400 mg) and benzyl 2-methyl-5-(1-methyl-3,6-dihydro-2H-pyridin-4-yl)benzoate (4.26 g, 13.3 mmol) gave 2-methyl-5-(1-methyl-4-piperidyl)benzoic acid (2.10 g, 68%) as a white solid. ¹H NMR (500 MHz, DMSO-d₆) δ 7.67 (d, J=1.5, 1H), 7.22 (d, J=7.5, 1H), 7.15 (d, J=7.5, 1H), 3.05-3.04 (m, 2H), 2.56-2.55 (m, 1H), 2.46 (s, 3H), 2.39 (s, 3H), 2.28-2.27 (m, 2H), 1.83-1.74 (m, 4H). LC-MS (Method B): R_{T}=0.55, m/z=232.6 [M−H]⁻.

### Step C: N-[(1R)-1-(3-bromo-4-methoxy-phenyl)ethyl]-2-methyl-5-(1-methyl-4-piperidyl)benzamide

Using General Procedure 1 with (1R)-1-(3-bromo-4-methoxy-phenyl)ethanamine hydrochloride salt (0.50 g, 1.88 mmol) (Example 180, Step C) and 2-methyl-5-(1-methyl-4-piperidyl)benzoic acid (481 mg, 2.06 mmol), gave N-[(1R)-1-(3-bromo-4-methoxy-phenyl)ethyl]-2-methyl-5-(1-methyl-4-piperidyl)benzamide (184 mg, 21%) as an off-white solid. ¹H NMR (500 MHz, DMSO-d₆) δ 8.68 (d, J=8.0, 1H), 7.60 (d, J=2.0, 1H), 7.37 (dd, J=8.5, 2.0, 1H), 7.20 (m, 2H), 7.14 (br s, 1H), 7.09 (d, J=8.5, 1H), 5.08 (quin, J=7.0, 1H), 3.84 (s, 3H), 3.41 (br s, 2H), 3.00 (br s, 2H), 2.79 (br s, 3H), 2.53 (m, 1H), 2.24 (s, 3H), 1.96 (m, 2H), 1.82 (m, 2H), 1.41 (d, J=7.0, 3H). LC-MS (Method B): R_{T}=4.32, m/z=443.8/445.8 [M−H]⁻.

### Example 300: N-[(1R)-1-[4-Methoxy-3-(1-methylpyrazol-4-yl)phenyl]ethyl]-2-methyl-5-(1-methyl-4-piperidyl)benzamide

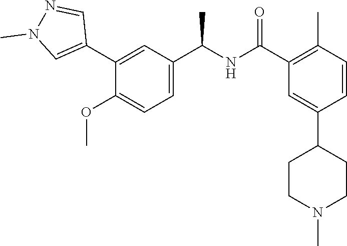 [SMILES: COc1ccc([C@@H](C)NC(=O)c2cc(C3CCN(C)CC3)ccc2C)cc1-c1cnn(C)c1]

Using General Procedure 1 with (1R)-1-[4-methoxy-3-(1-methylpyrazol-4-yl)phenyl]ethanamine hydrochloride salt (92 mg, 344 μmol)—prepared in a similar manner to (1R)-1-[2-methoxy-5-(1-methylpyrazol-4-yl)phenyl]ethanamine hydrochloride salt (Example 239, Step D)—and 2-methyl-5-(1-methyl-4-piperidyl)benzoic acid (88 mg, 378 μmol) (Example 299, Step B) with purification by FCC (eluting with 0-100% MeOH in DCM followed by 1N NH₃ in MeOH) gave N-[(1R)-1-[4-methoxy-3-(1-methylpyrazol-4-yl)phenyl]ethyl]-2-methyl-5-(1-methyl-4-piperidyl)benzamide (73 mg, 45%) as a white crystalline solid. ¹H NMR (500 MHz, DMSO-d₆) δ 8.61 (d, J=8.0, 1H), 8.08 (s, 1H), 7.85 (s, 1H), 7.62 (d, J=2.0, 1H), 7.22-7.18 (m, 2H), 7.16-7.14 (m, 2H), 7.03 (d, J=8.5, 1H), 5.11 (quin, J=7.0, 1H), 3.88 (s, 3H), 3.86 (s, 3H), 2.86 (br d, J=11.0, 2H), 2.24 (s, 3H), 2.18 (s, 3H), 1.96 (td, J=11.0, 2.0, 2H), 1.73-1.61 (m, 4H), 1.44 (d, J=7.0, 3H) LC-MS (Method B): R_{T}=4.09, m/z=445.9 [M−H]⁻.

### Further Examples

The following examples were prepared in a similar manner to N-[(1R)-1-[4-methoxy-3-(1-methylpyrazol-4-yl)phenyl]ethyl]-2-methyl-5-(1-methyl-4-piperidyl)benzamide (Example 300).

| Example | Structure | Name and Analytical Data |
|---|---|---|
| 301 | 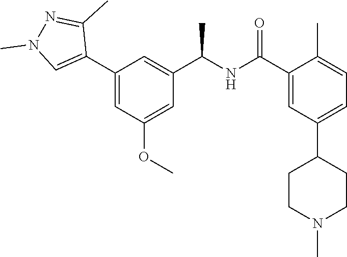 [SMILES: COc1cc(-c2cn(C)nc2C)cc([C@@H](C)NC(=O)c2cc(C3CCN(C)CC3)ccc2C)c1] | N-[(1R)-1-[3-(1,3-Dimethylpyrazol-4-yl)-5- methoxy-phenyl]ethyl]-2-methyl-5-(1- methyl-4-piperidyl)benzamide ¹H NMR (500 MHz, CDCl₃) δ 7.44 (s, 1H), 7.21 (s, 1H), 7.19-7.12 (m, 2H), 6.99 (s, 1H), 6.83 (br d, J = 7.9, 2H), 5.97 (br d, J = 7.9, 1H), 5.32 (t, J = 7.3, 1H), 3.87 (s, 3H), 3.84 (s, 3H), 3.00- 2.91 (m, 2H), 2.50-2.40 (m, 7H), 2.31 (s, 3H), 2.09-1.96 (m, 2H), 1.86-1.68 (m, 4H), 1.61 (d, J = 6.7, 3H). LC-MS (Method B): R_{T} = 3.90 m/z = 459.9 [M − H]⁻. |
| 302 | 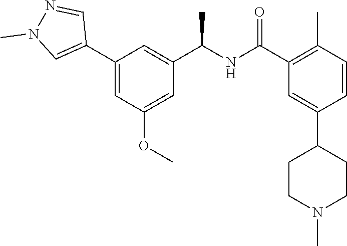 [SMILES: COc1cc(-c2cnn(C)c2)cc([C@@H](C)NC(=O)c2cc(C3CCN(C)CC3)ccc2C)c1] | N-[(1R)-1-[3-Methoxy-5-(1-methylpyrazol-4- yl)phenyl]ethyl]-2-methyl-5-(1-methyl-4- piperidyl)benzamide ¹H NMR (500 MHz, DMSO-d₆) δ 8.65 (d, J = 8.0, 1H), 8.12 (s, 1H), 7.84 (s, 1H), 7.21- 7.15 (m, 4H), 7.00 (br s, 1H), 6.82 (br s, 1H), 5.10 (quin, J = 7.0, 1H), 3.87 (s, 3H), 3.79 (s, 3H), 2.87 (br d, J = 11.0, 2H), 2.45 (m, 1H), 2.25 (s, 3H), 2.20 (s, 3H), 1.97 (m, 2H), 1.74 (m, 2H), 1.64 (m, 2H), 1.44 (d, J = 7.0, 1H). LC-MS (Method B): R_{T} = 3.97, m/z = 445.8 [M − H]⁻. |

### Example 303: N-[(1R)-1-[3-Methoxy-5-(1-methylpyrazol-4-yl)phenyl]ethyl]-2-methyl-5-(4-piperidyl)benzamide

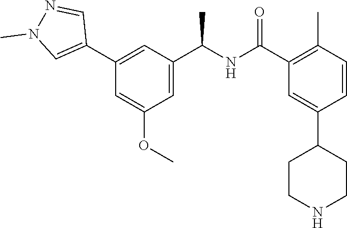 [SMILES: COc1cc(-c2cnn(C)c2)cc([C@@H](C)NC(=O)c2cc(C3CCNCC3)ccc2C)c1]

Using General Procedure 4 with tert-butyl 4-[3-[[(1R)-1-[3-methoxy-5-(1-methylpyrazol-4-yl)phenyl]ethyl]carbamoyl]-4-methyl-phenyl]piperidine-1-carboxylate (140 mg, 263 μmol)—prepared in a similar manner to N-[(1R)-1-[4-methoxy-3-(1-methylpyrazol-4-yl)phenyl]ethyl]-2-methyl-5-(1-methyl-4-piperidyl)benzamide (Example 300)—gave N-[(1R)-1-[3-methoxy-5-(1-methylpyrazol-4-yl)phenyl]ethyl]-2-methyl-5-(4-piperidyl)benzamide (55 mg, 44%) as a white crystalline solid. ¹H NMR (500 MHz, DMSO-d₆) δ 8.66 (br d, J=8.0, 1H), 8.12 (s, 1H), 7.84 (s, 1H), 7.21-7.15 (m, 4H), 7.00, (s, 1H), 6.82 (s, 1H), 5.11 (quin, J=7.0, 1H), 3.87 (s, 3H), 3.79 (s, 3H), 3.57 (s, 1H), 3.02 (br d, J=12.0, 2H), 2.57-2.56 (m, 3H), 2.25 (s, 3H), 1.68 (br d, J=12.0, 2H), 1.51-1.50 (m, 2H), 1.44 (d, J=7.0, 3H). LC-MS (Method B): R_{T}=4.83, m/z=431.8 [M−H]⁻.

### Further Examples

The following examples were prepared in a similar manner to N-[(1R)-1-[3-methoxy-5-(1-methylpyrazol-4-yl)phenyl]ethyl]-2-methyl-5-(4-piperidyl)benzamide (Example 303).

| Example | Structure | Name and Analytical Data |
|---|---|---|
| 304 | 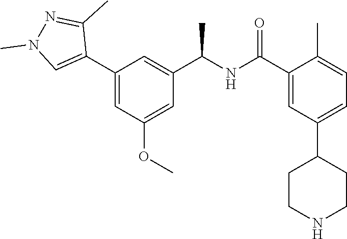 [SMILES: COc1cc(-c2cn(C)nc2C)cc([C@@H](C)NC(=O)c2cc(C3CCNCC3)ccc2C)c1] | N-[(1R)-1-[3-(1,3-Dimethylpyrazol-4-yl)-5- methoxy-phenyl]ethyl]-2-methyl-5-(4- piperidyl)benzamide ¹H NMR (500 MHz, CDCl₃) δ 7.44 (s, 1H), 7.50 (s, 1H), 7.23-7.11 (m, 2H), 6.99 (s, 1H), 6.83 (br d, J = 4.9, 2H), 5.97 (br d, J = 7.6, 1H), 5.37- 5.27 (m, 1H), 3.87 (s, 3H), 3.84 (s, 3H), 3.17 (br d, J = 11.6, 2H), 2.72 (br t, J = 11.6, 2H), 2.64-2.51 (m, 1H), 2.40 (s, 6H), 1.78 (br d, J = 12.5, 2H), 1.68-1.54 (m, 5H). LC- MS (Method B): R_{T} = 5.28 m/z = 445.8 [M − H]⁻. |

### Example 305: N-[(1R)-1-(4-Hydroxy-3-methoxy-phenyl)ethyl]-2-methyl-5-(4-methylpiperazin-1-yl)benzamide

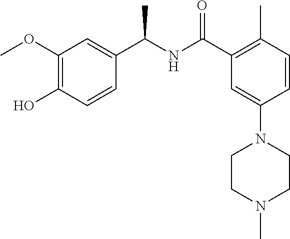 [SMILES: COc1cc([C@@H](C)NC(=O)c2cc(N3CCN(C)CC3)ccc2C)ccc1O]

Using General Procedure 5 with palladium hydroxide, 20% on carbon (146 mg, 208 μmol) and N-[(1R)-1-(4-benzyloxy-3-methoxy-phenyl)ethyl]-2-methyl-5-(4-methylpiperazin-1-yl)benzamide (304 mg, 642 μmol) (Example 184) gave N-[(1R)-1-(4-hydroxy-3-methoxy-phenyl)ethyl]-2-methyl-5-(4-methylpiperazin-1-yl)benzamide (246 mg, 95%) as a yellow foam. ¹H NMR (500 MHz, CDCl₃) δ 7.10-7.03 (m, 1H), 6.93-6.81 (m, 5H), 5.89 (brd, J=7.9, 1H), 5.25 (quin, J=7.2, 1H), 3.89 (s, 3H), 3.22-3.08 (m, 4H), 2.62-2.53 (m, 4H), 2.34 (s, 3H), 2.29 (m, 3H), 1.57 (d, J=7.2, 3H). LC-MS (Method A): R_{T}=3.83, m/z=384.7 [M+H]⁺.

### Example 306: N-[(1R)-1-[3-Methoxy-4-(1-methylpyrazol-4-yl)phenyl]ethyl]-2-methyl-5-(4-methylpiperazin-1-yl)benzamide

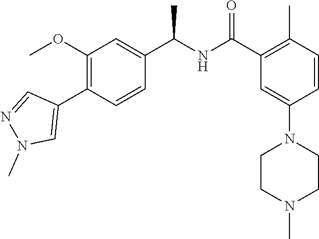 [SMILES: COc1cc([C@@H](C)NC(=O)c2cc(N3CCN(C)CC3)ccc2C)ccc1-c1cnn(C)c1]

### Step A: [2-Methoxy-4-[(1R)-1-[[2-methyl-5-(4-methylpiperazin-1-yl)benzoyl]amino]ethyl]phenyl] trifluoromethanesulfonate

N-[(1R)-1-(4-Hydroxy-3-methoxy-phenyl)ethyl]-2-methyl-5-(4-methylpiperazin-1-yl)benzamide (156 mg, 407 μmol) (Example 305) was dissolved in DCM (3.7 mL) and triethylamine (227 μL, 1.63 mmol) added. The clear yellow solution was cooled in an ice bath and 1,1,1-trifluoro-N-phenyl-N-(trifluoromethylsulfonyl)methanesulfonamide (174 mg, 488 μmol) added in one portion as a solid. The mixture was stirred in the ice bath for 2 hours then allowed to warm to room temperature and stirred overnight. Mixture was then partitioned between DCM (50 mL) and water (20 mL). The organic layer was separated, dried over sodium sulfate, filtered, and evaporated under reduced pressure to give crude material. Purification by FCC (eluting with 0-100% MeOH in ethyl acetate) gave [2-methoxy-4-[(1R)-1-[[2-methyl-5-(4-methylpiperazin-1-yl)benzoyl]amino]ethyl]phenyl]trifluoromethanesulfonate (192 mg, 92%) as a white foam. LC-MS (Method A): R_{T}=4.41, m/z=516.7 [M+H]⁺.

### Step B: N-[(1R)-1-[3-Methoxy-4-(1-methylpyrazol-4-yl)phenyl]ethyl]-2-methyl-5-(4-methylpiperazin-1-yl)benzamide

Using General Procedure 2 with 1-methyl-4-(4,4,5,5-tetramethyl-1,3,2-dioxaborolan-2-yl)pyrazole (42 mg, 203 μmol) and [2-methoxy-4-[(1R)-1-[[2-methyl-5-(4-methylpiperazin-1-yl)benzoyl]amino]ethyl]phenyl] trifluoromethanesulfonate (95 mg, 184 μmol) at 60° C. for 2 hours with purification by FCC (eluting with 0-100% MeOH in ethyl acetate) gave N-[(1R)-1-[3-Methoxy-4-(1-methylpyrazol-4-yl)phenyl]ethyl]-2-methyl-5-(4-methylpiperazin-1-yl)benzamide (75 mg, 82%) as a beige foam. ¹H NMR (500 MHz, CDCl₃) δ 7.84 (s, 1H), 7.83-7.79 (m, 1H), 7.52-7.47 (m, 1H), 7.08 (d, J=8.4, 1H), 6.98 (dd, J=2.7, 3.9, 2H), 6.94-6.91 (m, 1H), 6.90-6.84 (m, 1H), 6.84-6.80 (m, 1H), 5.99-5.93 (m, 1H), 5.36-5.30 (m, 1H), 3.94 (s, 3H), 3.92 (s, 3H), 3.20-3.12 (m, 4H), 2.59-2.51 (m, 4H), 2.34 (s, 3H), 2.31 (s, 3H), 1.61 (d, J=6.9, 3H). LC-MS (Method A): R_{T}=3.05, m/z=448.8 [M+H]⁺.

### Example 307: 2-Methyl-N-[(1R)-1-[3-(4-methylpiperazine-1-carbonyl)phenyl]ethyl]-5-(4-methylpiperazin-1-yl)benzamide

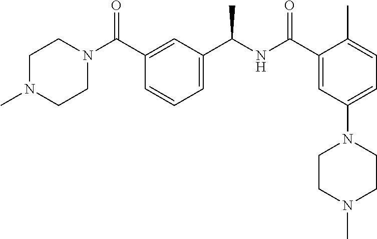 [SMILES: Cc1ccc(N2CCN(C)CC2)cc1C(=O)N[C@H](C)c1cccc(C(=O)N2CCN(C)CC2)c1]

### Step A: 1-[(1R)-1-(3-Bromophenyl)ethyl]-2,5-dimethyl-pyrrole

(1R)-1-(3-Bromophenyl)ethanamine (6.20 g, 31.0 mmol), 2,5-hexanedione (5.31 g, 46.5 mmol) and scandium trifluoromethanesulfonate (457 mg, 929 μmol) were stirred overnight to afford a brown viscous solution. The reaction was quenched with 2N HCl (75 mL), extracted with diethyl ether (2×75 mL), dried (MgSO₄) and solvent evaporated to afford a black liquid. This was purified by FCC (eluting with 5% diethyl ether in petroleum ether) to afford 1-[(1R)-1-(3-bromophenyl)ethyl]-2,5-dimethyl-pyrrole (4.30 g, 50%) as a clear liquid. LC-MS (Method B): R_{T}=4.68: m/z=non seen [M−H]⁻.

### Step B: 3-[(1R)-1-(2,5-Dimethylpyrrol-1-yl)ethyl]benzoic Acid

1-[(1R)-1-(3-Bromophenyl)ethyl]-2,5-dimethyl-pyrrole (4.29 g, 15.4 mmol) was dissolved in THF (40 mL) and cooled to −78° C. under nitrogen. Once cooled n-butyllithium solution in hexanes (1.9 M, 8.12 mL) was slowly added to afford a red solution, the mixture was stirred for 10 mins before being quenched by bubbling carbon dioxide through the reaction until a colourless solution was observed. The mixture was then stirred for 10 minutes before being quenched with 1N HCl (50 mL), extracted with diethyl ether (2×50 ml), dried (MgSO₄) and solvent evaporated to afford 3-[(1R)-1-(2,5-dimethylpyrrol-1-yl)ethyl]benzoic acid (3.75 g, 100%) as an orange gum. This was used in Step C directly with no purification.

### Step C: Benzyl 3-[(1R)-1-(2,5-dimethylpyrrol-1-yl)ethyl]benzoate

3-[(1R)-1-(2,5-Dimethylpyrrol-1-yl)ethyl]benzoic acid (3.75 g, 15.0 mmol), potassium carbonate (2.77 g, 20.0 mmol) and benzyl bromide (2.64 g, 15.0 mmol) were added to DMF (40 mL) and stirred for 2 hours. The reaction was quenched with water (100 mL), extracted with diethyl ether (2×75 mL), dried (MgSO₄) and solvent evaporated to afford a dark gum. Purification by FCC (eluting with 5-20% diethyl ether in petroleum ether) afforded a solid which was triturated with petroleum ether to afford a solid which was filtered to give benzyl 3-[(1R)-1-(2,5-dimethylpyrrol-1-yl)ethyl]benzoate (2.50 g, 48%) as a white solid. LC-MS (Method B): R_{T}=4.80: m/z=331.8 [M−H]⁻.

### Step D: Benzyl 3-[(1R)-1-aminoethyl]benzoate

Benzyl 3-[(1R)-1-(2,5-dimethylpyrrol-1-yl)ethyl]benzoate (2.30 g, 6.90 mmol), hydroxylamine hydrochloride (7.19 g, 103 mmol) and triethylamine (3.85 mL, 27.6 mmol) were added to ethanol (40 mL) and water (10 mL) and the mixture was heated at reflux for 24 hours. The reaction was evaporated to 50% volume, quenched with 2N NaOH (50 mL), extracted with diethyl ether (2×75 mL), dried (MgSO₄) and solvent evaporated to afford benzyl 3-[(1R)-1-aminoethyl]benzoate (1.69 g, 67%) as a yellow liquid. LC-MS (Method B): R_{T}=3.73: m/z=254.6 [M−H]⁻.

### Step E: Benzyl 3-[(1R)-1-[[2-methyl-5-(4-methylpiperazin-1-yl)benzoyl]amino]ethyl]benzoate

Using General Procedure 1 with benzyl 3-[(1R)-1-aminoethyl]benzoate (1.69 g, 6.62 mmol) and 2-methyl-5-(4-methylpiperazin-1-yl)benzoic acid hydrochloride salt (2.02 g, 8.61 mmol) (Example 21, Step B) gave benzyl 3-[(1R)-1-[[2-methyl-5-(4-methylpiperazin-1-yl)benzoyl]amino]ethyl]benzoate (2.20 g, 69%) as a clear gum. LC-MS (Method B): R_{T}=3.90, m/z=470.9 [M−H]⁻.

### Step F: 3-[(1R)-1-[[2-Methyl-5-(4-methylpiperazin-1-yl)benzoyl]amino]ethyl]benzoic Acid

Using General Procedure 5 with benzyl 3-[(1R)-1-[[2-methyl-5-(4-methylpiperazin-1-yl)benzoyl]amino]ethyl]benzoate (2.00 g, 4.24 mmol) and palladium hydroxide, 20% on carbon (119 mg, 848 μmol) gave 3-[(1R)-1-[[2-methyl-5-(4-methylpiperazin-1-yl)benzoyl]amino]ethyl]benzoic acid (1.42 g, 87%) as a beige solid. LC-MS (Method B): R_{T}=0.53, m/z=380.8 [M−H]⁻.

### Step G: 2-Methyl-N-[(1R)-1-[3-(4-methylpiperazine-1-carbonyl)phenyl]ethyl]-5-(4-methylpiperazin-1-yl)benzamide

Using General Procedure 1 with 3-[(1R)-1-[[2-methyl-5-(4-methylpiperazin-1-yl)benzoyl]amino]ethyl]benzoic acid (200 mg, 524 μmol), and N-methylpiperazine (78 mg, 786 μmol) gave 2-methyl-N-[(1R)-1-[3-(4-methylpiperazine-1-carbonyl)phenyl]ethyl]-5-(4-methylpiperazin-1-yl)benzamide (210 mg, 86%) as a white foam. ¹H NMR (500 MHz, CDCl₃) δ 7.49-7.35 (m, 3H), 7.30 (td, J=1.4, 7.5, 1H), 7.08 (d, J=8.2, 1H), 6.92 (d, J=2.4, 1H), 6.90-6.85 (m, 1H), 5.98 (br d, J=7.6, 1H), 5.33 (quin, J=7.2, 1H), 3.80 (br s, 2H), 3.55-3.36 (m, 2H), 3.21-3.12 (m, 4H), 2.62-2.53 (m, 4H), 2.55-2.35 (brm, 4H), 2.35 (s, 3H), 2.33-2.29 (m, 6H), 1.59 (d, J=7.0, 3H). LC-MS (Method B): R_{T}=2.74, m 1Z=462.9 [M−H]⁻.

### Further Examples

The following examples were prepared in a similar manner to 2-methyl-N-[(1R)-1-[3-(4-methylpiperazine-1-carbonyl)phenyl]ethyl]-5-(4-methylpiperazin-1-yl)benzamide (Example 307), using the required commercially available amine in Step G. Examples which did not solid maternal after Step G were treated with 6N HCl in propan-2-ol, then concentrated and triturated with an appropriate solvent to product as a hydrochloride salt.

| Example | Structure | Name and Analytical Data |
|---|---|---|
| 308 | 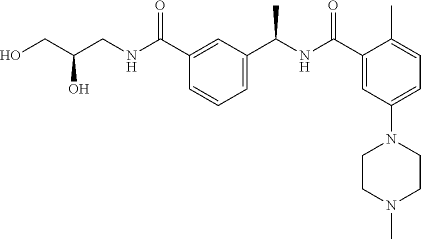 [SMILES: Cc1ccc(N2CCN(C)CC2)cc1C(=O)N[C@H](C)c1cccc(C(=O)NC[C@@H](O)CO)c1] | N-[(1R)-1-[3-[[(2R)-2,3- Dihydroxypropyl]carbamoyl] phenyl]ethyl]-2-methyl-5-(4- methylpiperazin-1-yl)benzamide hydrochloride salt ¹H NMR (500 MHz, DMSO-d₆) δ 8.74 (br d, J = 7.9, 1H), 8.44 (br t, J = 5.6, 1H), 7.95 (s, 1H), 7.75 (br d, J = 7.6, 1H), 7.54 (br d, J = 7.3, 1H), 7.48-7.37 (m, 1H), 7.12 (d, J = 8.2, 1H), 7.03-6.92 (m, 2H), 5.15 (br t, J = 7.3, 1H), 3.81 (br m, 2H), 3.72- 3.57 (m, 1H), 3.48 (br m, 2H), 3.43- 3.32 (m, 3H), 3.24-3.04 (m, 5H), 2.81 (d, J = 4.3, 3H), 2.19 (s, 3H), |
| | | 1.46 (d, J = 7.0, 3H). LC-MS (Method |
| | | B): R_{T} = 2.68, m/z = 453.9 [M − H]⁻. |
| 309 | 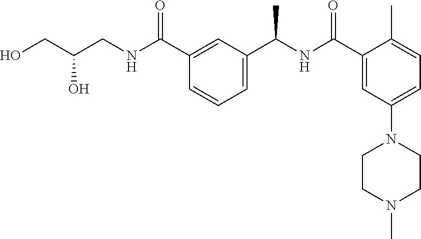 [SMILES: Cc1ccc(N2CCN(C)CC2)cc1C(=O)N[C@H](C)c1cccc(C(=O)NC[C@H](O)CO)c1] | N-[(1R)-1-[3-[[(2S)-2,3- Dihydroxypropyl]carbamoyl] phenyl]ethyl]-2-methyl-5-(4- methylpiperazin-1-yl)benzamide ¹H NMR (500 MHz, DMSO-d₆) δ 8.70 (d, J = 8.0, 1H), 8.38 (t, J = 5.5, 1H), 7.90 (br s, 1H), 7.72 (d, J = 8.0, 1H), 7.54 (d, J = 8.0, 1H), 7.43 (t, J = 7.5, 1H), 7.06 (d, J = 8.0, 1H), 6.90 (dd, J = 8.0, 3.0, 1H), 6.87 (d, J = 3.0, 1H), 5.14 (quin, J = 7.0, 1H), 4.83 (d, J = 5.0, 1H), 4.58 (t, J = 6.0, 1H), 3.64 (m, 1H), 3.41 (m, 1H), 3.35 (t, J = 5.5, 2H), 3.20 (m, 1H), 3.11 (m, 4H), 2.45 (m, 4H), 2.22 (s, 3H), 2.16 (s, |
| | | 3H), 1.45 (d, J = 7.0, 3H). LC-MS |
| | | (Method B): R_{T} = 2.72, m/z = 453.9 |
| | | [M − H]⁻. |
| 310 | 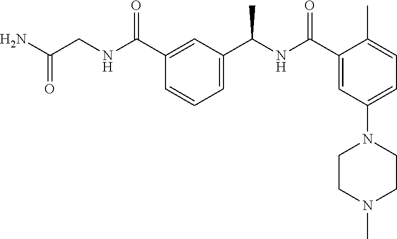 [SMILES: Cc1ccc(N2CCN(C)CC2)cc1C(=O)N[C@H](C)c1cccc(C(=O)NCC(N)=O)c1] | N-[(1R)-1-[3-[(2-Amino-2-oxo- ethyl)carbamoyl]phenyl]ethyl]-2- methyl-5-(4-methylpiperazin-1- yl)benzamide hydrochloride salt ¹H NMR (500 MHz, DMSO-d₆) δ 8.76 (d, J = 8.2, 1H), 8.72-8.65 (m, 1H), 7.96 (s, 1H), 7.77 (d, J = 7.6, 1H), 7.56 (d, J = 7.6, 1H), 7.49-7.39 (m, 2H), 7.12 (d, J = 8.5, 1H), 7.08- 7.02 (m, 1H), 7.02-6.92 (m, 2H), 5.15-5.06 (m, 1H), 3.87-3.77 (m, 4H), 3.53-3.42 (m, 2H), 3.18-3.06 (m, 4H), 2.80 (d, J = 4.9, 3H), 2.19 (s, 3H), 1.46 (d, J = 7.0, 3H). LC- MS (Method B): R_{T} = 2.68, m/z = 436.8 [M − H]⁻. |
| 311 | 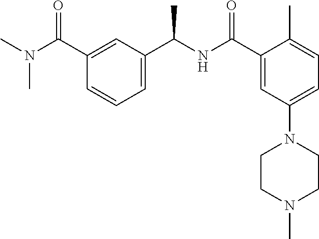 [SMILES: Cc1ccc(N2CCN(C)CC2)cc1C(=O)N[C@H](C)c1cccc(C(=O)N(C)C)c1] | N-[(1R)-1-[3- (Dimethylcarbamoyl)phenyl]ethyl]- 2-methyl-5-(4-methylpiperazin- 1-yl)benzamide ¹H NMR (500 MHz, DMSO-d₆) δ 8.67 (d, J = 8.0, 1H), 7.46-7.43 (m, 2H), 7.40 (t, J = 7.5, 1H), 7.26 (dt, J = 7.5, 1.5, 1H), 7.06 (d, J = 8.0, 1H), 6.91 (dd, J = 8.5, 2.5, 1H), 6.85 (d, J = 2.5, 1H), 5.14 (quin, J = 7.0, 1H), 3.10 (m, 4H), 2.99 (br s, 3H), 2.91 (br s, 3H), 2.45 (m, 4H), 2.22 (s, 3H), 2.15 (s, 3H), 1.44 (d, J = 7.0, 3H). LC-MS (Method B): R_{T} = 2.91, m/z = 407.8 [M − H]⁻. |
| 312 | 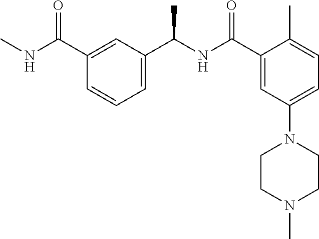 [SMILES: CNC(=O)c1cccc([C@@H](C)NC(=O)c2cc(N3CCN(C)CC3)ccc2C)c1] | 2-Methyl-N-[(1R)-1-[3- (methylcarbamoyl)phenyl]ethyl]- 5-(4-methylpiperazin-1- yl)benzamide ¹H NMR (500 MHz, DMSO-d₆) δ 8.37 (d, J = 8.0, 1H), 8.42 (m, 1H), 7.89 (s, 1H), 7.69 (d, J = 8.0, 1), 7.53 (d, J = 7.5, 1H), 7.42 (t, J = 7.5, 1H), 7.08 (d, J = 7.5, 1H), 6.93 (dd, J = 8.5, 3.0, 1H), 6.90 (d, J = 3.0, 1H), 5.14 (quin, J = 7.0, 1H), 3.20 (m, 4H), 2.79 (d, J = 4.5, 3H), 2.74 (m, 4H), 2.44 (br s, 3H), 2.17 (s, 3H), 1.45 (d, J = 7.0, 3H). LC-MS (Method B): R_{T} = 2.94, m/z = 393.8 [M − H]⁻. |

### Example 313: N-[(1R)-1-[3-(2-Hydroxyethylcarbamoyl)phenyl]ethyl]-2-methyl-5-(4-methylpiperazin-1-yl)benzamide

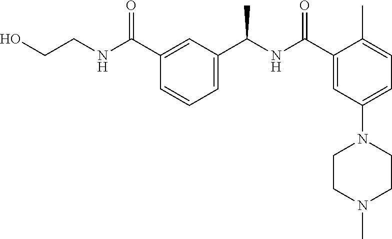 [SMILES: Cc1ccc(N2CCN(C)CC2)cc1C(=O)N[C@H](C)c1cccc(C(=O)NCCO)c1]

Using General Procedure 4 with N-[(1R)-1-[3-[2-[tert-butyl(dimethyl)silyl]oxyethylcarbamoyl]phenyl]ethyl]-2-methyl-5-(4-methylpiperazin-1-yl)benzamide (120 mg, 223 μmol)—prepared in a similar manner to 2-methyl-N-[(1R)-1-[3-(4-methylpiperazine-1-carbonyl)phenyl]ethyl]-5-(4-methylpiperazin-1-yl)benzamide (Example 307)—gave N-[(1R)-1-[3-(2-hydroxyethylcarbamoyl)phenyl]ethyl]-2-methyl-5-(4-methylpiperazin-1-yl)benzamide (41 mg, 41%) as a white crystalline solid. ¹H NMR (500 MHz, DMSO-d₆) δ 8.63 (d, J=8.0, 1H), 8.35 (t, J=5.5, 1H), 7.84 (br s, 1H), 7.66 (d, J=7.5, 1H), 7.48 (d, J=8.5, 1H), 7.36 (t, J=7.5, 1H), 7.00 (d, J=8.5, 1H), 6.84 (dd, J=8.0, 2.5, 1H), 6.81 (d, J=2.5, 1H), 5.08 (quin, J=7.0, 1H), 4.67 (t, J=5.5, 1H), 3.46 (q, J=5.5, 2H), 3.28 (peak obscured by solvent, should be 2H), 3.05 (m, 4H), 2.40 (m, 4H), 2.17 (s, 3H), 2.10 (s, 3H), 1.39 (d, J=7.0, 3H). LC-MS (Method B): R_{T}=2.72, m/z=423.9 [M−H]⁻.

### Example 314: 2-Methyl-N-[(1R)-1-[3-[methyl-[2-(methylamino)ethyl]carbamoyl]phenyl]ethyl]-5-(4-methylpiperazin-1-yl)benzamide Dihydrochloride Salt

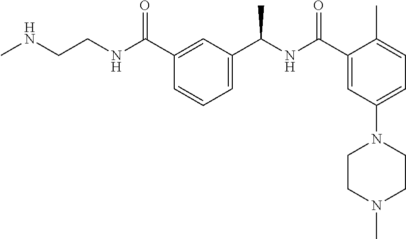 [SMILES: CNCCNC(=O)c1cccc([C@@H](C)NC(=O)c2cc(N3CCN(C)CC3)ccc2C)c1]

Using General Procedure 5 with benzyl N-methyl-N-[2-[methyl-[3-[(1R)-1-[[2-methyl-5-(4-methylpiperazin-1-yl)benzoyl] amino]ethyl]benzoyl]amino]ethyl]carbamate (300 mg, 512 μmol)—prepared in a similar manner to 2-methyl-N-[(1R)-1-[3-(4-methylpiperazine-1-carbonyl)phenyl]ethyl]-5-(4-methylpiperazin-1-yl)benzamide (Example 307)—and palladium hydroxide, 20% on carbon (21 mg, 154 mmol) gave a yellow gum. The gum was dissolved in MeOH (3 mL) to this was added diethyl ether to afford a cloudy solution. 6.0 N HCl in propan-2-ol was added (0.3 mL) to afford a gummy solid which was stirred under nitrogen for 1 hour and then filtered under nitrogen to give 2-methyl-N-[(1R)-1-[3-[methyl-[2-(methylamino)ethyl]carbamoyl]phenyl]ethyl]-5-(4-methylpiperazin-1-yl)benzamide hydrochloride salt (240 mg, 89%) as a white solid. ¹H NMR (500 MHz, DMSO-d₆) δ 11.24 (br s, 1H), 8.74 (br d, J=7.9, 1H), 7.65-7.52 (m, 1H), 7.51-7.45 (m, 1H), 7.42 (br d, J=4.0, 2H), 7.12 (d, J=8.5, 1H), 6.99 (dd, J=2.6, 8.4, 1H), 6.96-6.91 (m, 1H), 5.14 (br t, J=7.3, 1H), 4.95 (brs, 3H), 3.87-3.70 (m, 4H), 3.53-3.44 (m, 2H), 3.20-3.09 (m, 6H), 2.92 (s, 3H), 2.80 (d, J=4.6, 3H), 2.18 (s, 3H), 1.46 (d, J=7.0, 3H). LC-MS (Method B): R_{T}=3.09, m/z=450.9 [M−H]⁻.

### Example 315: N-[(1R)-1-[3-(3-Hydroxyprop-1-ynyl)phenyl]ethyl]-2-methyl-5-(4-methylpiperazin-1-yl)benzamide

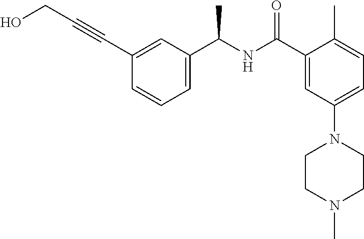 [SMILES: Cc1ccc(N2CCN(C)CC2)cc1C(=O)N[C@H](C)c1cccc(C#CCO)c1]

### Step A: N-[(1R)-1-(3-Bromophenyl)ethyl]-1,1-diphenyl-methanimine

Benzophenoneimine (2.61 g, 14.4 mmol) was added to a solution of (1R)-1-(3-bromophenyl)ethanamine (2.94 g, 14.7 mmol) in DCM (65 mL) and the reaction mixture stirred at RT for 48 hours. The reaction was concentrated in vacuo, then water (75 mL) and petroleum ether (75 mL) were added. The phases were separated, and the organic phase washed with brine (75 mL), dried (Na₂SO₄), and filtered. The solvent was removed in vacuo and purification by FCC (eluting with 0-10% ethyl acetate in petroleum ether) gave N-[(1R)-1-(3-bromophenyl)ethyl]-1,1-diphenyl-methanimine (3.37 g, 63%) as a pale yellow oil which solidified on standing to give a white solid. ¹H NMR (500 MHz, CDCl₃) δ 7.86 (m, 2H), 7.52-7.45 (m, 4H), 7.39-7.32 (m, 4H), 7.28 (m, 1H), 7.16 (t, J=8.0, 1H), 7.11 (m, 2H), 4.49 (q, J=6.5, 1H), 1.44 (d, J=6.5, 3H).

### Step B: 3-[3-[(1R)-1-(Benzhydrylideneamino)ethyl]phenyl]prop-2-yn-1-ol

N-[(1R)-1-(3-Bromophenyl)ethyl]-1,1-diphenyl-methanimine (0.50 g, 1.37 mmol), propargyl alcohol (115.42 mg, 2.06 mmol) and bis(triphenylphosphine)palladium(II) chloride (96.3 mg, 137 μmol) were added to piperidine (5 mL) and stirred at 70° C. for 1 hour to afford a black solution. The mixture was evaporated to dryness, quenched with water (50 mL), extracted with diethyl ether (2×50 mL), dried (MgSO₄) and solvent evaporated to afford a black gum. Purification by FCC (eluting with 40-60% diethyl ether in petroleum ether) afforded 3-[3-[(1R)-1-(benzhydrylideneamino)ethyl]phenyl]prop-2-yn-1-ol (180 mg, 39%) as a yellow gum. This was used directly in Step C.

### Step C: 3-[3-[(1R)-1-Aminoethyl]phenyl]prop-2-yn-1-ol

3-[3-[(1R)-1-(Benzhydrylideneamino)ethyl]phenyl]prop-2-yn-1-ol (180 mg, 530 μmol) was added to THF (5 mL) and 5N hydrogen chloride aq. (5 mL) and stirred overnight. The mixture was diluted with water (20 mL) and extracted with diethyl ether (50 mL). The aqueous layer was basified to pH 11 with solid KOH, extracted with diethyl ether (2×50 mL), dried (MgSO₄) and solvent evaporated to afford a yellow gum. This was passed down an SCX column eluting with MeOH and then 3.5N NH₃ in MeOH to afford 3-[3-[(1R)-1-aminoethyl]phenyl]prop-2-yn-1-ol (52 mg, 56%) as a yellow gum. LC-MS (Method B): R_{T}=2.35, m/z=176.4 [M−H]⁻.

### Step D: N-[(1R)-1-[3-(3-hydroxyprop-1-ynyl)phenyl]ethyl]-2-methyl-5-(4-methylpiperazin-1-yl)benzamide

Using General Procedure 1 with 3-[3-[(1R)-1-aminoethyl]phenyl]prop-2-yn-1-ol (52 mg, 295 μmol) and 2-methyl-5-(4-methylpiperazin-1-yl)benzoic acid hydrochloride salt (80 mg, 295 μmol) (Example 21, Step B) gave N-[(1R)-1-[3-(3-hydroxyprop-1-ynyl)phenyl]ethyl]-2-methyl-5-(4-methylpiperazin-1-yl)benzamide (43 mg, 37%) as a white solid. ¹H NMR (500 MHz, CDCl₆) δ 7.46 (s, 1H), 7.35-7.28 (m, 3H), 7.08 (d, J=8.2, 1H), 6.93 (d, J=2.4, 1H), 6.91-6.87 (m, 1H), 5.97 (br d, J=8.2, 1H), 5.28 (quin, J=7.2, 1H), 4.48 (s, 2H), 3.25-3.07 (m, 4H), 2.58 (m, 4H), 2.35 (s, 3H), 2.31 (s, 3H), 1.62-1.43 (m, 3H). LC-MS (Method B): R_{T}=2.86, m/z=390.8 [M−H]⁻.

### Example 316: 2-(4-Methylpiperazin-1-yl)-N-[(1R)-1-(1-naphthyl)ethyl]pyrimidine-4-carboxamide Hydrochloride Salt

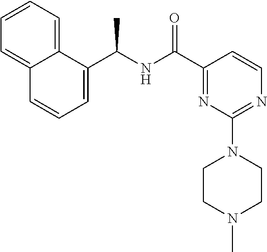 [SMILES: C[C@@H](NC(=O)c1ccnc(N2CCN(C)CC2)n1)c1cccc2ccccc12]

### Step A: 2-Methylsulfinyl-N-[(1R)-1-(1-naphthyl)ethyl]pyrimidine-4-carboxamide

2-Methylsulfanyl-N-[(1R)-1-(1-naphthyl)ethyl]pyrimidine-4-carboxamide (1.42 g, 4.39 mmol)—prepared in a similar manner to N-[(1R)-1-(1-naphthyl)ethyl]-3-(1-piperidyl)benzamide (Example 1)—was added to DCM (20 mL). To this was added slowly 3-chloroperbenzoic acid (909 mg, 5.27 mmol) and the mixture was stirred for 20 mins. The mixture was evaporated so that most of the DCM was removed, diluted with diethyl ether, and then quenched with sat. aq. Na₂CO₃ solution (50 mL). This was then extracted with diethyl ether (2×50 mL), dried (MgSO₄) and solvent removed in vacuo to afford 2-methylsulfinyl-N-[(1R)-1-(1-naphthyl)ethyl]pyrimidine-4-carboxamide (1.37 g, 92%) as a white foam. LC-MS (Method B): R_{T}=2.98, m/z=338.5 [M−H]⁻.

### Step B: 2-(4-Methylpiperazin-1-yl)-N-[(1R)-1-(1-naphthyl)ethyl]pyrimidine-4-carboxamide

2-Methylsulfinyl-N-[(1R)-1-(1-naphthyl)ethyl]pyrimidine-4-carboxamide (145 mg, 427 μmol) and N-methylpiperazine (171 mg, 1.71 mmol) were added to DMF and heated at 60° C. for 2 hours. The reaction was quenched with water (50 mL), extracted with diethyl ether (2×60 mL), dried (MgSO₄) and solvent evaporated to afford a yellow gum. Purification by FCC (eluting with ethyl acetate and then 20% MeOH in ethyl acetate) afford a slightly yellow gum/foam. This was dissolved in diethyl ether (20 mL), then added 4N HCl in 1,4-dioxane (25 mg, 692 μmol) to afford a solid which was stirred and filtered under nitrogen to afford 2-(4-methylpiperazin-1-yl)-N-[(1R)-1-(1-naphthyl)ethyl]pyrimidine-4-carboxamide (134 mg, 94%) as a white solid. ¹H NMR (500 MHz, DMSO-d₆) δ 9.13 (br d, J=8.5, 1H), 8.65 (d, J=4.9, 1H), 8.20 (d, J=8.5, 1H), 7.96 (d, J=7.6, 1H), 7.91-7.81 (m, 1H), 7.66 (d, J=7.0, 1H), 7.62-7.47 (m, 3H), 7.24 (d, J=4.6, 1H), 6.04-5.92 (m, 1H), 5.04-4.81 (m, 2H), 3.50 (br d, J=11.6, 2H), 3.46-3.32 (m, 2H), 3.06 (br d, J=12.2, 2H), 2.80 (br d, J=4.0, 3H), 1.69 (d, J=7.0, 3H). LC-MS (Method B): R_{T}=3.66, m/z=374.7 [M−H]⁻.

### Further Examples

The following examples were prepared in a similar manner to 2-(4-methylpiperazin-1-yl)-N-[(1R)-1-(1-naphthyl)ethyl]pyrimidine-4-carboxamide hydrochloride salt (Example 316), using the required commercially available secondary amine.

| Example | Structure | Name and Analytical Data |
|---|---|---|
| 317 | 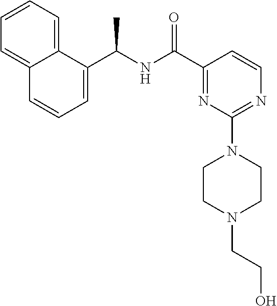 [SMILES: C[C@@H](NC(=O)c1ccnc(N2CCN(CCO)CC2)n1)c1cccc2ccccc12] | 2-[4-(2-Hydroxyethyl)piperazin-1- yl]-N-[(1R)-1-(1- naphthyl)ethyl]pyrimidine-4- carboxamide hydrochloride salt ¹H NMR (500 MHz, DMSO-d₆) δ 9.12 (d, J = 8.5, 1H), 8.65 (d, J = 4.9, 1H), 8.21 (d, J = 8.2, 1H), 7.96 (d, J = 7.3, 1H), 7.91-7.81 (m, 1H), 7.66 (d, J = 7.0, 1H), 7.63-7.47 (m, 3H), 7.23 (d, J = 4.6, 1H), 6.04-5.92 (m, 1H), 4.89 (br s, 2H), 3.86-3.79 (m, 1H), 3.63-3.57 (m, 4H), 3.52-3.43 (m, 2H), 3.22 (q, J = 5.0, 2H), 3.17- 3.08 (m, 2H), 1.69 (d, J = 7.0, 3H). LC-MS (Method B): R_{T} = 3.77, m/z = 404.7 [M − H]⁻. |

### Example 318: 2-[(3S)-3-Aminopyrrolidin-1-yl]-N-[(1R)-1-(1-naphthyl)ethyl]pyrimidine-4-carboxamide

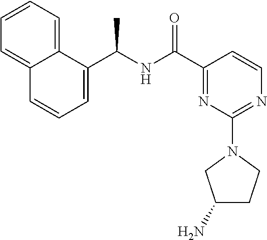 [SMILES: C[C@@H](NC(=O)c1ccnc(N2CC[C@H](N)C2)n1)c1cccc2ccccc12]

Using General Procedure 4 with tert-butyl N-[(3S)-1-[4-[[(1R)-1-(1-naphthyl)ethyl]carbamoyl]pyrimidin-2-yl]pyrrolidin-3-yl]carbamate (150 mg, 325 μmol)—prepared in a similar manner to 2-(4-methylpiperazin-1-yl)-N-[(1R)-1-(1-naphthyl)ethyl]pyrimidine-4-carboxamide hydrochloride salt (Example 312)—gave 2-[(3S)-3-aminopyrrolidin-1-yl]-N-[(1R)-1-(1-naphthyl)ethyl]pyrimidine-4-carboxamide (52 mg, 42%) as a white crystalline solid.

¹H NMR (500 MHz, DMSO-d₆) δ 8.83 (d, J=8.0, 1H), 8.53 (d, J=5.0, 1H), 8.20 (d, J=8.0, 1H), 7.97 (d, J=7.5, 1H), 7.86 (d, J=8.0, 1H), 7.65 (d, J=7.5, 1H), 7.60-7.50 (m, 3H), 7.05 (d, J=5.0, 1H), 5.94 (quin, J=7.5, 1H), 3.75 (br s, 1H), 3.60 (m, 2H), 3.57 (m, 1H), 3.24 (br s, 1H), 2.04 (m, 1H), 1.88 (br s, 2H), 1.71 (m, 1H), 1.67 (d, J=7.0, 3H). LC-MS (Method B): R_{T}=3.80, m/z=360.7 [M−H]⁻.

### Example 319: 2-[(3R)-3-Aminopyrrolidin-1-yl]-N-[(1R)-1-(1-naphthyl)ethyl]pyrimidine-4-carboxamide Hydrochloride Salt

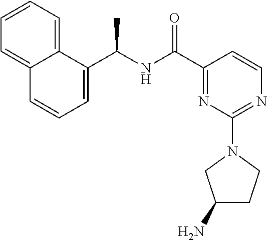 [SMILES: C[C@@H](NC(=O)c1ccnc(N2CC[C@@H](N)C2)n1)c1cccc2ccccc12]

tert-Butyl N-[(3R)-1-[4-[[(1R)-1-(1-naphthyl)ethyl]carbamoyl]pyrimidin-2-yl]pyrrolidin-3-yl]carbamate (270 mg, 585 μmol)—prepared in a similar manner to 2-(4-methylpiperazin-1-yl)-N-[(1R)-1-(1-naphthyl)ethyl]pyrimidine-4-carboxamide hydrochloride salt (Example 312)—was dissolved in diethyl ether (1 mL), to this was added 6N HCl in propan-2-ol (585 μmol) and the mixture was stirred overnight to afford a semi solid solution. Water (20 mL) and diethyl ether (30 mL) were added, and the mixture was stirred for 10 mins, the organic layer was further extracted with water (20 mL). The combined aqueous layer was basified to pH 10 with solid NaOH, this was then re-extracted with diethyl ether (2×30 mL), dried (MgSO₄) and solvent removed in vacuo to afford a gum. This was dissolved in DCM/diethyl ether, this was then acidified with 4N HCl in 1,4-dioxane to afford a semi solid. The mixture was evaporated to dryness to give a solid which was slurried in diethyl ether and filtered under nitrogen to afford 2-[(3R)-3-aminopyrrolidin-1-yl]-N-[(1R)-1-(1-naphthyl)ethyl]pyrimidine-4-carboxamide hydrochloride salt (185 mg, 79%) as a yellow solid. ¹H NMR (500 MHz, DMSO-d₆) δ 8.90 (d, J=8.5, 1H), 8.59 (d, J=4.9, 1H), 8.19 (d, J=8.2, 1H), 7.97 (d, J=7.9, 1H), 7.91-7.81 (m, 1H), 7.66 (d, J=7.3, 1H), 7.61-7.48 (m, 3H), 7.15 (d, J=4.9, 1H), 6.01-5.90 (m, 1H), 4.36 (br s, 2H), 3.99-3.87 (m, 1H), 3.86-3.65 (m, 4H), 2.38-2.24 (m, 1H), 2.24-2.05 (m, 1H), 1.67 (d, J=6.7, 3H). LC-MS (Method B): R_{T}=3.67 m/z=360.7 [M−H]⁻.

### Example 320: 6-(4-Methylpiperazin-1-yl)-N-[(1R)-1-(1-naphthyl)ethyl]pyrazine-2-carboxamide Hydrochloride Salt

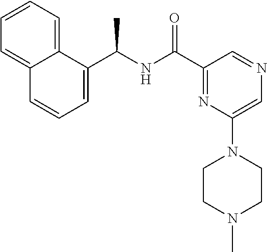 [SMILES: C[C@@H](NC(=O)c1cncc(N2CCN(C)CC2)n1)c1cccc2ccccc12]

6-Chloro-N-[(1R)-1-(1-naphthyl)ethyl]pyrazine-2-carboxamide (279 mg, 895 μmol)—prepared in a similar manner to N-[(1R)-1-(1-Naphthyl)ethyl]-3-(1-piperidyl)benzamide (Example 1)—and N-methylpiperazine (269 mg, 2.68 mmol) were added to DMF (5 mL) and heated at 60° C. for 3 hours. The reaction was diluted with water (50 mL), extracted with diethyl ether (2×50 mL), dried (MgSO₄) and solvent evaporated to afford a yellow gum. This was purified by FCC (eluting with 0-20% MeOH in ethyl acetate) to afford a light-yellow gum. This was dissolved in diethyl ether and then 4N HCl in 1,4-dioxane was added to afford a bright yellow solid, this was stirred for 10 minutes then filtered under nitrogen to afford 6-(4-methylpiperazin-1-yl)-N-[(1R)-1-(1-naphthyl)ethyl]pyrazine-2-carboxamide hydrochloride salt (165 mg, 45%) as a yellow solid. ¹H NMR (500 MHz, DMSO-d₆) δ 11.22 (br s, 1H), 8.99 (br d, J=8.5, 1H), 8.61 (s, 1H), 8.45 (s, 1H), 8.22 (br d, J=8.5, 1H), 7.96 (br d, J=7.9, 1H), 7.90-7.80 (m, 1H), 7.66 (br d, J=7.0, 1H), 7.63-7.47 (m, 3H), 6.08-5.91 (m, 1H), 4.78-4.54 (m, 2H), 3.50 (br d, J=11.3, 2H), 3.46-3.34 (m, 4H), 3.10 (br d, J=9.8, 3H), 1.68 (br d, J=6.4, 3H). LC-MS (Method B): R_{T}=3.43 m/z=374.6 [M−H]⁻.

### Biological Data

Compounds of the invention were tested in a papain-like protease inhibition assay to investigate the mechanism of action of the compounds. Results are reported as the concentration of test article required to inhibit enzyme activity by 50% (IC50). Compounds exhibited IC50 values consistent with potent, specific inhibition of the tested papain-like protease.

Inhibition of papain-like protease enzyme function was performed at 37° C. in buffer at pH 7.5 (50 mM HEPES, 0.1 mg/ml BSA, 5 mM DTT), containing 60 nM papain-like protease, 50 μM Z-Arg-Leu-Arg-Gly-Gly-AMC (Z-RLRGG-AMC), and a range of concentrations of compound. Enzyme, buffer, and inhibitor compound were incubated for 10 minutes at 37° C. before the addition of Z-RLRGG-AMC. Fluorescence was measured (excitation 355 nm, emission 460 nm, gain 800) using a BMG LABTECH FLUOstar Omega microplate reader every minute for 30 minutes. IC50s were determined from the average increase in OD per minute versus the Log 10 concentration of compound using GraphPad Prism.

TABLE 1a

| | SARS-CoV-2 |
|---|---|
| | PLpro IC50 |
| Example | (μM) |
| 1 | F |
| 2 | A |
| 3 | F |
| 4 | B |
| 5 | F |
| 6 | D |
| 7 | F |
| 8 | E |
| 9 | B |
| 10 | E |
| 11 | A |
| 12 | B |
| 13 | C |
| 14 | F |
| 15 | C |
| 16 | B |
| 17 | B |
| 18 | D |
| 19 | A |
| 20 | B |
| 21 | A |
| 22 | B |
| 23 | A |
| 24 | C |
| 25 | D |
| 26 | E |
| 27 | A |
| 28 | E |
| 29 | B |
| 30 | C |
| 31 | B |
| 32 | B |
| 33 | C |
| 34 | C |
| 35 | E |
| 36 | A |
| 37 | D |
| 38 | D |
| 39 | A |
| 40 | A |
| 41 | B |
| 42 | A |
| 43 | A |
| 44 | A |
| 45 | A |
| 46 | A |
| 47 | A |
| 48 | B |
| 49 | A |
| 50 | A |
| 51 | A |
| 52 | A |
| 53 | A |
| 54 | C |
| 55 | A |
| 56 | A |
| 57 | A |
| 58 | A |
| 59 | C |
| 60 | B |
| 61 | A |
| 62 | A |
| 63 | A |
| 64 | A |
| 65 | A |
| 66 | A |
| 67 | A |
| 68 | A |
| 69 | C |
| 70 | A |
| 71 | A |
| 72 | A |
| 73 | A |
| 74 | A |
| 75 | A |
| 76 | A |
| 77 | A |
| 78 | A |
| 79 | A |
| 80 | A |
| 81 | A |
| 82 | E |
| 83 | B |
| 84 | D |
| 85 | C |
| 86 | A |
| 87 | A |
| 88 | A |
| 89 | B |
| 90 | A |
| 91 | A |
| 92 | A |
| 93 | A |
| 94 | A |
| 95 | A |
| 96 | C |
| 97 | A |
| 98 | A |
| 99 | A |
| 100 | A |
| 101 | A |
| 102 | A |
| 103 | B |
| 104 | B |
| 105 | B |
| 106 | B |
| 107 | B |
| 108 | A |
| 109 | C |
| 110 | F |
| 111 | A |
| 112 | A |
| 113 | D |
| 114 | B |
| 115 | A |
| 116 | C |
| 117 | B |
| 118 | A |
| 119 | E |
| 120 | A |
| 121 | A |
| 122 | B |
| 123 | A |
| 124 | A |
| 125 | A |
| 126 | A |
| 127 | E |
| 128 | A |
| 129 | B |
| 130 | C |
| 131 | F |
| 132 | E |
| 133 | B |
| 134 | A |
| 135 | — |
| 136 | — |
| 137 | F |
| 138 | F |
| 139 | D |
| 140 | C |
| 141 | F |
| 142 | F |
| 143 | D |
| 144 | C |
| 145 | E |
| 146 | A |
| 147 | B |
| 148 | F |
| 149 | E |
| 150 | E |
| 151 | F |
| 152 | E |
| 153 | D |
| 154 | A |
| 155 | A |
| 156 | A |
| 157 | A |
| 158 | B |
| 159 | F |
| 160 | E |

TABLE 1b

| | SARS-CoV-2 |
|---|---|
| | PLpro IC50 |
| Example | (μM) |
| 161 | D |
| 162 | A |
| 163 | B |
| 164 | E |
| 165 | A |
| 166 | C |
| 167 | A |
| 168 | B |
| 169 | D |
| 170 | B |
| 171 | A |
| 172 | A |
| 173 | F |
| 174 | E |
| 175 | F |
| 176 | E |
| 177 | D |
| 178 | D |
| 179 | F |
| 180 | A |
| 181 | A |
| 182 | B |
| 183 | D |
| 184 | A |
| 185 | C |
| 186 | A |
| 187 | A |
| 188 | A |
| 189 | A |
| 190 | B |
| 191 | E |
| 192 | A |
| 193 | E |
| 194 | E |
| 195 | A |
| 196 | A |
| 197 | A |
| 198 | A |
| 199 | A |
| 200 | A |
| 201 | A |
| 202 | B |
| 203 | A |
| 204 | A |
| 205 | A |
| 206 | A |
| 207 | A |
| 208 | A |
| 209 | A |
| 210 | B |
| 211 | A |
| 212 | A |
| 213 | A |
| 214 | A |
| 215 | A |
| 216 | A |
| 217 | A |
| 218 | A |
| 219 | A |
| 220 | A |
| 221 | A |
| 222 | A |
| 223 | A |
| 224 | B |
| 225 | C |
| 226 | C |
| 227 | A |
| 228 | A |
| 229 | A |
| 230 | A |
| 231 | A |
| 232 | B |
| 233 | A |
| 234 | A |
| 235 | A |
| 236 | A |
| 237 | B |
| 238 | C |
| 239 | A |
| 240 | A |
| 241 | B |
| 242 | A |
| 243 | A |
| 244 | A |
| 245 | A |
| 246 | A |
| 247 | A |
| 248 | A |
| 249 | A |
| 250 | A |
| 251 | A |
| 252 | A |
| 253 | A |
| 254 | A |
| 255 | A |
| 256 | A |
| 257 | A |
| 258 | A |
| 259 | A |
| 260 | A |
| 261 | A |
| 262 | A |
| 263 | A |
| 264 | A |
| 265 | A |
| 266 | A |
| 267 | A |
| 268 | A |
| 269 | — |
| 270 | A |
| 271 | B |
| 272 | A |
| 273 | B |
| 274 | A |
| 275 | A |
| 276 | A |
| 277 | A |
| 278 | A |
| 279 | A |
| 280 | A |
| 281 | A |
| 282 | A |
| 283 | A |
| 284 | A |
| 285 | A |
| 286 | A |
| 287 | A |
| 288 | A |
| 289 | A |
| 290 | A |
| 291 | B |
| 292 | A |
| 293 | A |
| 294 | B |
| 295 | C |
| 296 | A |
| 297 | A |
| 298 | A |
| 299 | A |
| 300 | A |
| 301 | A |
| 302 | A |
| 303 | A |
| 304 | A |
| 305 | B |
| 306 | A |
| 307 | A |
| 308 | C |
| 309 | B |
| 310 | B |
| 311 | D |
| 312 | C |
| 313 | D |
| 314 | D |
| 315 | B |
| 316 | E |
| 317 | F |
| 318 | E |
| 319 | F |
| 320 | E |

Key to tables: The following letters in Tables 1a and 1b above represent the IC50 values in μM: A≤2, B≤5, C≤10, D≤20, E≤100 and F>100.

Cytotoxicity of compounds of the invention was evaluated in human Hep G2 cells (ATCC HB-8065) seeded at a density of 2×10⁴ cells per well and incubated for 24 hours at 37° C., 5% CO2. Cells were exposed to 100 μM solution of test article. After 24-hour exposure, the viability of the cells was determined using CellTiter-Glo®) (Promega, WI, USA) according to the manufacturer's instructions. Results are reported as percentage cell viability at tested concentration.

TABLE 2

| | % Cell |
|---|---|
| Example | Viability |
| 2 | 20.3 |
| 4 | 56.8 |
| 6 | 0.6 |
| 9 | 96.5 |
| 11 | 95.6 |
| 12 | 91.1 |
| 13 | 103.8 |
| 15 | 76.1 |
| 16 | 1.1 |
| 17 | 34.7 |
| 18 | 119.3 |
| 19 | 1.0 |
| 20 | 94.3 |
| 21 | 56.9 |
| 22 | 65.5 |
| 23 | 87.3 |
| 24 | 70.7 |
| 25 | 95.2 |
| 26 | 82.8 |
| 27 | 1.0 |
| 29 | 100 |
| 30 | 102.3 |
| 31 | 90.6 |
| 32 | 103.5 |
| 33 | 95.5 |
| 34 | 97.8 |
| 36 | 98.0 |
| 39 | 90.5 |
| 40 | 0.9 |
| 41 | 110.1 |
| 42 | 64.1 |
| 43 | 71.3 |
| 44 | 61.0 |
| 45 | 59.5 |
| 46 | 86.9 |
| 47 | 54.0 |
| 48 | 63.6 |
| 49 | 82.7 |
| 50 | 2.3 |
| 52 | 49.0 |
| 53 | 82.4 |
| 54 | 76.0 |
| 55 | 1.0 |
| 56 | 75.1 |
| 57 | 0.9 |
| 58 | 86.7 |
| 59 | 86.0 |
| 60 | 74.3 |
| 61 | 69.2 |
| 62 | 56.6 |
| 63 | 75.6 |
| 64 | 78.6 |
| 65 | 77.6 |
| 66 | 100.8 |
| 67 | 58.7 |
| 68 | 48.9 |
| 69 | 107.0 |
| 70 | 59.1 |
| 71 | 68.4 |
| 72 | 79.0 |
| 73 | 0.6 |
| 74 | 0.6 |
| 75 | 123.6 |
| 76 | 81.2 |
| 77 | 1.0 |
| 78 | 1.3 |
| 79 | 52.9 |
| 80 | 79.5 |
| 81 | 94.4 |
| 85 | 85.1 |
| 86 | 72.1 |
| 87 | 74.6 |
| 88 | 78.8 |
| 89 | 96.2 |
| 90 | 103.1 |
| 91 | 27.1 |
| 92 | 76.9 |
| 93 | 41.9 |
| 94 | 17.3 |
| 95 | 88.5 |
| 96 | 97.3 |
| 97 | 72.1 |
| 98 | 98.5 |
| 99 | 104.6 |
| 100 | 65.9 |
| 101 | 88.1 |
| 102 | 75.9 |
| 103 | 91.4 |
| 104 | 82.7 |
| 105 | 113.3 |
| 106 | 87.4 |
| 107 | 100.7 |
| 108 | 2.1 |
| 109 | 72.9 |
| 111 | 74.8 |
| 112 | 41.0 |
| 113 | 72.7 |
| 114 | 59.0 |
| 115 | 26.7 |
| 116 | 66.6 |
| 117 | 61.1 |
| 118 | 6.6 |
| 120 | 96.2 |
| 121 | 75.1 |
| 122 | 95.4 |
| 123 | 32.9 |
| 124 | 37.4 |
| 125 | 55.8 |
| 126 | 62.8 |
| 128 | 1.1 |
| 129 | 95.5 |
| 130 | 1.0 |
| 133 | 0.1 |
| 134 | 0.8 |
| 140 | 0.8 |
| 144 | 1.6 |
| 146 | 0.2 |
| 147 | 0.8 |
| 154 | 2.1 |
| 155 | 0.6 |
| 156 | 0.6 |
| 157 | 1.5 |
| 158 | 0.8 |
| 159 | 81.5 |
| 160 | 101.8 |
| 161 | 97.9 |
| 162 | 104.9 |
| 163 | 122.4 |
| 165 | 132 |
| 166 | 100.9 |
| 167 | 110.4 |
| 168 | 111.2 |
| 170 | 107.9 |
| 172 | 104.6 |
| 178 | 77.2 |
| 180 | 93.4 |
| 181 | 18.9 |
| 182 | 104.8 |
| 184 | 51.3 |
| 185 | 5.6 |
| 186 | 1.0 |
| 187 | 44.0 |
| 188 | 84.9 |
| 189 | 1.0 |
| 190 | 0.9 |
| 192 | 6.3 |
| 195 | 41.5 |
| 196 | 4.7 |
| 197 | 44.7 |
| 198 | 1.6 |
| 199 | 0.7 |
| 200 | 24.3 |
| 201 | 1.0 |
| 202 | 72.5 |
| 203 | 0.9 |
| 204 | 81.4 |
| 205 | 105.8 |
| 206 | 0.8 |
| 207 | 103.7 |
| 208 | 87.8 |
| 209 | 86.3 |
| 210 | 64.0 |
| 211 | 86.0 |
| 212 | 96.8 |
| 213 | 67.2 |
| 214 | 101.2 |
| 215 | 80.1 |
| 216 | 0.6 |
| 217 | 61.7 |
| 218 | 1.5 |
| 219 | 88.7 |
| 220 | 80.7 |
| 221 | 74.9 |
| 222 | 7.4 |
| 223 | 59.8 |
| 224 | 105.0 |
| 225 | 95.8 |
| 226 | 91.4 |
| 227 | 89.9 |
| 228 | 69.9 |
| 229 | 66.7 |
| 230 | 38.3 |
| 231 | 1.6 |
| 232 | 75.2 |
| 233 | 88.9 |
| 234 | 0.9 |
| 235 | 90.6 |
| 236 | 79.5 |
| 237 | 87.9 |
| 238 | 119.7 |
| 239 | 101.6 |
| 240 | 64.3 |
| 241 | 84.2 |
| 242 | 62.4 |
| 243 | 70.9 |
| 244 | 67.1 |
| 245 | 58.4 |
| 246 | 81.4 |
| 247 | 56.7 |
| 248 | 55.3 |
| 249 | 0.5 |
| 250 | 93.0 |
| 251 | 63.2 |
| 252 | 49.4 |
| 253 | 89.3 |
| 254 | 57.0 |
| 255 | 70.3 |
| 256 | 70.1 |
| 258 | 81.8 |
| 259 | 111.1 |
| 260 | 91.1 |
| 261 | 49.2 |
| 262 | 42.4 |
| 263 | 56.4 |
| 264 | 68.5 |
| 265 | 40.7 |
| 266 | 61.1 |
| 267 | 81.0 |
| 268 | 75.0 |
| 270 | 22.6 |
| 271 | 95.2 |
| 272 | 74.3 |
| 273 | 92.6 |
| 274 | 38.7 |
| 275 | 80.7 |
| 276 | 81.6 |
| 280 | 76.7 |
| 282 | 100.0 |
| 284 | 101.3 |
| 285 | 72.2 |
| 286 | 50.3 |
| 287 | 43.4 |
| 288 | 47.2 |
| 289 | 59.5 |
| 290 | 72.9 |
| 291 | 97.4 |
| 292 | 88.9 |
| 293 | 102.3 |
| 294 | 120.9 |
| 295 | 107.9 |
| 296 | 76.1 |
| 297 | 61.1 |
| 298 | 43.9 |
| 299 | 76.2 |
| 300 | 74.7 |
| 301 | 77.5 |
| 302 | 67.1 |
| 303 | 73.2 |
| 306 | 88.4 |
| 308 | 126.8 |
| 309 | 118.2 |
| 310 | 124.4 |
| 311 | 130.7 |
| 312 | 78.3 |
| 315 | 122.7 |

Anti-viral potency of compounds of the invention was assessed in 96-well plates using VERO E6 cells. To generate EC50 and EC90 values for each compound, cells were treated in minimal medium at a range of compound concentrations. The plates were then incubated at 37° C. with 5% CO2 for 2 hours. The minimal media containing the experimental compounds and the control media was then removed. Wells were then treated with 50 μL minimal media containing SARS-CoV-2 (MOI of 0.005), 100 μL 2× semi-solid media and then 50 μL minimal media containing experimental compounds and control media, as appropriate. After 48 hours, 4% paraformaldehyde was added to each well and the plate incubated for 1 hour at room temperature. The medium was removed, cells were stained with crystal violet. Cells were washed three times with water and cytopathic viral activity was determined by measuring absorbance of each well at 590 nm using a Varioskan LUX microplate reader (Thermo Fisher Scientific). At all concentrations, treatment of cells was performed alongside 2 μM of CP-100356—a known efflux pump inhibitor.

TABLE 3

| | SARS-COV- |
|---|---|
| Example | 2 EC50 (μM) |
| 9 | B |
| 11 | A |
| 61 | A |
| 64 | A |
| 72 | A |
| 102 | A |
| 205 | A |
| 212 | A |
| 215 | A |
| 217 | A |
| 219 | A |
| 220 | B |
| 221 | A |
| 227 | A |
| 230 | A |
| 233 | A |
| 235 | A |
| 239 | A |
| 244 | A |
| 245 | A |
| 251 | A |
| 252 | A |
| 253 | A |
| 260 | B |
| 261 | B |
| 266 | A |
| 272 | A |
| 280 | A |
| 289 | A |
| 293 | A |
| 296 | B |
| 300 | A |
| 306 | A |

Key to table: The following letters in Table 1 above represent the EC50 values in μM: A≤5, B≤15.

1. A compound of formula (I) or pharmaceutically acceptable salt thereof: 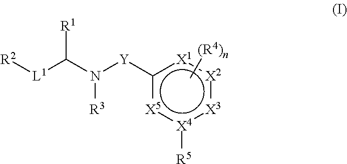
wherein
Y is —C(O)—, —C(S)—, —C(═NR⁶)—;
-L¹- is absent or a linker selected from C₁ alkylene, C₂-alkenylene, or C₂-alkynylene;
X¹ is absent or is selected from carbon and nitrogen;
X², X³ and X⁵ are each independently selected from carbon, nitrogen, oxygen and sulfur;
X⁴ is selected from carbon and nitrogen;
wherein when X¹ is carbon or nitrogen, X⁴ is carbon, X², X³ and X⁵ are each independently selected from carbon and nitrogen, and no more than two of X¹, X², X³ and X⁵ may be nitrogen,
wherein when X¹ is absent, no more than two of X², X³, X⁴ and X⁵ may be nitrogen and no more than one of X², X³ and X⁵ may be oxygen or sulfur;
R¹ is selected from the group comprising: C₁ or C₂ alkyl, C₁ or C₂ haloalkyl, and C₁ or C₂ alkylene-R^{1a}; wherein R^{1a} is selected from OR⁶, SR⁶, NR⁶R⁷, CO₂R⁶ and CONR⁶R⁶;
R² is selected from phenyl, 5- or 6-membered heteroaryl, 5- or 6-membered heterocycloalkyl or C₅ or C₆ cycloalkyl, and said phenyl, heteroaryl or cycloalkyl is optionally fused to or substituted with a group selected from phenyl, 5- or 6-membered heteroaryl, 5- or 6-membered heterocycloalkyl or C₅ or C₆ cycloalkyl; wherein any said phenyl or heteroaryl group is optionally substituted with at least one R⁸ group; or wherein any said heterocycloalkyl or cycloalkyl is optionally substituted with at least one R⁹ group;
R³, R⁶ and R¹¹ are each independently at each occurrence selected from the group comprising: H and C₁-C₆-alkyl;
R⁴ is independently at each occurrence selected from the group comprising: halo, C₁-C₆-alkyl, C₁-C₆-haloalkyl, C₁-C₆-alkylene-R¹⁰, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₃₋₆ cycloalkyl, C₂₋₆-alkenyl, C₂₋₆-alkynyl, phenyl and 5- or 6-membered heteroaryl;
R⁵ is selected from the group comprising: —C(O)NR⁶R¹⁴, —C(O)R¹², phenyl, 5- or 6-membered heteroaryl; 5-, 6-, 7-, 8-, 9- or 10-membered heterocycloalkyl and cyclopropyl; wherein any said phenyl or heteroaryl group is optionally substituted with at least one R⁸ group; or wherein any said heterocycloalkyl or cyclopropyl is optionally substituted with at least one R⁹ group;
wherein when X⁴ is nitrogen, R⁵ is selected such that R⁵ is attached to X⁴ via a carbon atom;
R⁷ is independently at each occurrence selected from the group comprising: H, C₁-C₆-alkyl, C(O)—C₁-C₆-alkyl and S(O)₂—C₁-C₆-alkyl;
R⁸ is independently at each occurrence selected from the group comprising: halo, C₁-C₆-alkyl, C₁-C₆-haloalkyl, C₁-C₆-alkylene-R¹⁰, —OR¹⁰, cyano, nitro, —NR⁶R⁷, —SR¹⁰, C(O)R¹⁰, C(O)OR¹⁰, C(O)NR⁶R¹⁰, —S(O)R¹⁰, —S(O)₂R¹⁰, —S(O)₂NR⁶R¹⁰, C₃₋₆ cycloalkyl, C₂₋₆-alkenyl, C₂₋₆-alkynyl, 5-, 6-, 7-, 8-, 9- or 10-membered heterocycloalkyl, phenyl and 5- or 6-membered heteroaryl; wherein where R⁸ is heterocycloalkyl, phenyl or heteroaryl, R⁸ is optionally substituted where chemically possible with one or more R^{8c} groups;
R^{8c} is independently selected at each occurrence from: halo, C₁-C₆-alkyl, C₁-C₆-haloalkyl, C₁-C₆-alkylene-R¹⁰, C₁-C₆-alkylene-NR⁶R¹⁰, —OR¹⁰, C(O)R¹⁰, C(O)OR¹⁰, C(O)NR⁶R¹⁰;
R⁹ is independently at each occurrence selected from the group comprising: ═O, ═S, halo, C₁-C₆-alkyl, C₁-C₆-haloalkyl, —OR¹⁰, cyano, nitro, —NR⁶R⁷, —NR¹¹R¹², —SR¹⁰, C(O)R¹⁰, C(O)OR¹⁰, C(O)NR⁶R¹⁰, —S(O)R¹⁰, —S(O)₂R¹⁰, —S(O)₂NR⁶R¹⁰, C₃₋₆ cycloalkyl, 4-, 5- or 6-membered heterocycloalkyl, C₂₋₆-alkenyl C₂₋₆-alkynyl and C₁-C₃-alkylene-R^{9a}; wherein R^{9a} is selected from OR⁶, SR⁶, S(O)₂R⁶, S(O)₂NR⁶R⁶, S(O)₂Ph, NR⁶R⁷, CO₂R⁶, CONR⁶R⁶, 4-, 5- or 6-membered heterocycloalkyl, and cyclopropyl;
R¹⁰ is independently selected at each occurrence from the group comprising: H, C₁-C₆-alkyl, C₁-C₆-haloalkyl, C₁-C₆-alkylene-R^{10a}, C₃₋₈ cycloalkyl, 4-, 5-, 6-, 7- or 8-membered heterocycloalkyl, phenyl and 5- or 6-membered heteroaryl; wherein R^{10a} is independently selected at each occurrence from C₃₋₈ cycloalkyl, OR⁶, SR⁶, S(O)₂R⁶, S(O)₂Ph, NR⁶R⁷, CO₂R⁶, CONR⁶R⁶, phenyl, 5- or 6-membered heteroaryl, and 5- or 6-membered heterocycloalkyl;
R¹² is 6-membered heterocycloalkyl; wherein said heterocycloalkyl is optionally substituted with at least one R¹³ group;
R¹³ is independently at each occurrence selected from: ═O, ═S, halo, C₁-C₆-alkyl, C₁-C₆-haloalkyl, —OR⁶, cyano, nitro, —NR⁶R⁷, —SR⁶, C(O)R⁶, C(O)OR⁶, C(O)NR⁶R⁶, —S(O)R⁶, —S(O)₂R⁶, —S(O)₂NR⁶R⁶, C₃₋₆ cycloalkyl, C₂₋₆-alkenyl, C₂₋₆-alkynyl and C₁-C₆-alkylene-R^{13a}; wherein R^{13a} is selected from OR⁶, SR⁶, S(O)₂R⁶, S(O)₂Ph, NR⁶R⁷, CO₂R⁶ and CONR⁶R⁶;
R¹⁴ is selected from H and C₁-C₆-alkylene-R^{14a}; wherein R^{14a} is selected from OR⁶, SR⁶, S(O)₂R⁶, S(O)₂Ph, NR⁶R⁷, CO₂R⁶ and CONR⁶R⁶; and
n is an integer selected from 0, 1, 2, 3 or 4;
wherein any aforementioned alkyl, alkenyl, alkynyl, haloalkyl, cycloalkyl, heterocycloalkyl, phenyl, heteroaryl, alkylene, alkenylene, alkynylene, C(O)-alkyl and S(O)₂-alkyl is optionally substituted, where chemically possible, by 1 to 4 substituents which are each independently selected at each occurrence from the group consisting of: ═O; ═NR^{a}, ═NOR^{a}, C₁-C₄-alkyl, halo, nitro, cyano, C₁-C₄-haloalkyl, C₂-C₄-alkenyl, C₂-C₄-alkynyl, NR^{a}R^{b}, S(O)₂R^{a}, S(O)R^{a}, S(O)(NR^{a})R^{a}, S(O)₂NR^{a}R^{a}, CO₂R^{a}, C(O)R^{a}, CONR^{a}R^{a}, OR^{a} and SR^{a};
wherein R^{a} is independently selected from H and C₁-C₄-alkyl; and R^{b} is independently selected from H, C₁-C₄-alkyl, C(O)—C₁-C₄-alkyl and S(O)₂—C₁-C₄-alkyl.

2. The compound of claim 1, wherein R⁵ is 5-, 6- or 7- or 8-membered heterocycloalkyl, wherein said heterocycloalkyl is optionally substituted with at least one R⁹ group, and wherein said heterocycloalkyl group includes at least two heteroatoms.

3. The compound of claim 1 or claim 2, wherein R⁵ is 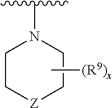
wherein Z is NR^{9b}, 0 or S(O)_{q};
x is selected from 0, 1, 2, 3, 4, 5 or 6; and
q is selected from 0, 1 or 2;
R^{9b} is selected from the group comprising: H, C₁-C₆-alkyl, C₁-C₆-haloalkyl, C(O)R¹⁰, C(O)OR¹⁰, C(O)NR⁶R¹⁰, —S(O)R¹⁰, —S(O)₂R¹⁰, —S(O)₂NR⁶R¹⁰, C₃₋₆ cycloalkyl, 4-, 5- or 6-membered heterocycloalkyl, C₂₋₆-alkenyl C₂₋₆-alkynyl, C₂-C₃-alkylene-R^{9a} and CH₂-cyclopropyl.

4. The compound of any of claims 1 to 3, wherein R⁵ is: 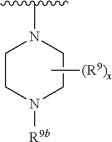
wherein x is selected from 0, 1, 2, 3, 4, 5 or 6; and
R^{9b} is selected from the group comprising: C₁-C₆-alkyl, C₁-C₆-haloalkyl, C(O)R⁶, C(O)OR⁶, C(O)NR⁶R⁶, —S(O)R⁶, —S(O)₂R⁶, —S(O)₂NR⁶R⁶, C₃₋₆ cycloalkyl, 4-, 5- or 6-membered heterocycloalkyl, C₂₋₆-alkenyl C₂₋₆-alkynyl, C₂-C₃-alkylene-R^{9a} and CH₂-cyclopropyl.

5. The compound of claim 1, wherein R⁵ is selected from the group comprising: —C(O)NR⁶R¹⁴ and —C(O)R¹².

6. The compound of any preceding claim, wherein X¹ is selected from carbon and nitrogen, X⁴ is carbon, X², X³ and X⁵ are each independently selected from carbon and nitrogen, and no more than two of X¹, X², X³ and X⁵ may be nitrogen.

7. The compound of any preceding claim, wherein the ring comprising X¹, X², X³, X⁴, and X⁵ is:

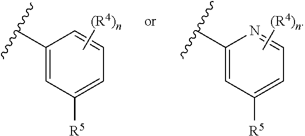

8. The compound of any preceding claim, wherein the ring comprising X¹, X², X³, X⁴, and X⁵ is: 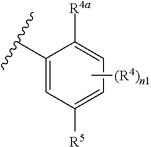
wherein R⁴ is independently selected from the group comprising: halo, C₁-C₆-alkyl, C₁-C₆-haloalkyl, C₁-C₆-alkylene-R¹⁰, —OR¹⁰, cyano, nitro, —NR⁶R⁷, —SR¹⁰, C(O)R⁶, C(O)OR⁶, C(O)NR⁶R⁶, —S(O)R¹⁰, —S(O)₂R¹⁰, —S(O)₂NR⁶R⁶, C₃₋₆ cycloalkyl, C₂₋₆-alkenyl, C₂₋₆-alkynyl, phenyl and 5- or 6-membered heteroaryl; and
n1 is an integer selected from 0, 1, 2 or 3.

9. The compound of claim 8, wherein R^{4a} is C₁₋₄ alkyl.

10. The compound of any preceding claim, wherein Y is —C(O)—.

11. The compound of any preceding claim, wherein R³ is H.

12. The compound of any preceding claim, wherein -L¹- is absent.

13. The compound of any preceding claim, wherein R¹ is C₁ or C₂ alkyl.

14. The compound of any preceding claim, wherein R² is phenyl, biphenyl, or naphthyl.

15. The compound of claim 1, wherein the compound is selected from the following compounds or pharmaceutically acceptable salts thereof:

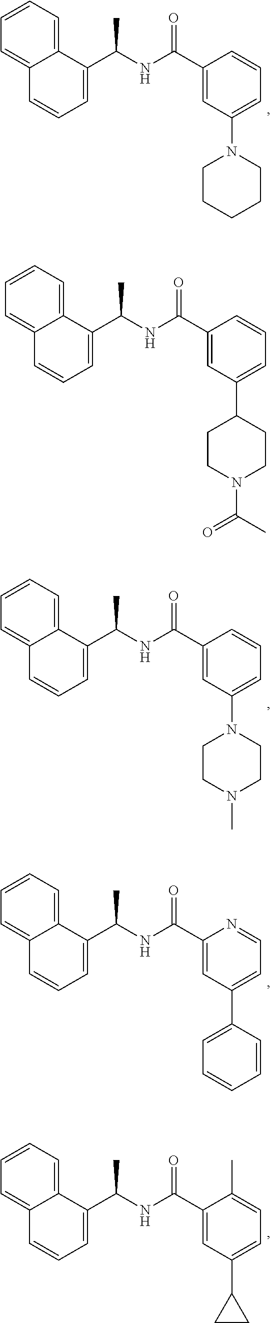 [SMILES: C[C@@H](NC(=O)c1cccc(N2CCCCC2)c1)c1cccc2ccccc12 ; CC(=O)N1CCC(c2cccc(C(=O)N[C@H](C)c3cccc4ccccc34)c2)CC1 ; C[C@@H](NC(=O)c1cccc(N2CCN(C)CC2)c1)c1cccc2ccccc12 ; C[C@@H](NC(=O)c1cc(-c2ccccc2)ccn1)c1cccc2ccccc12 ; Cc1ccc(C2CC2)cc1C(=O)N[C@H](C)c1cccc2ccccc12]

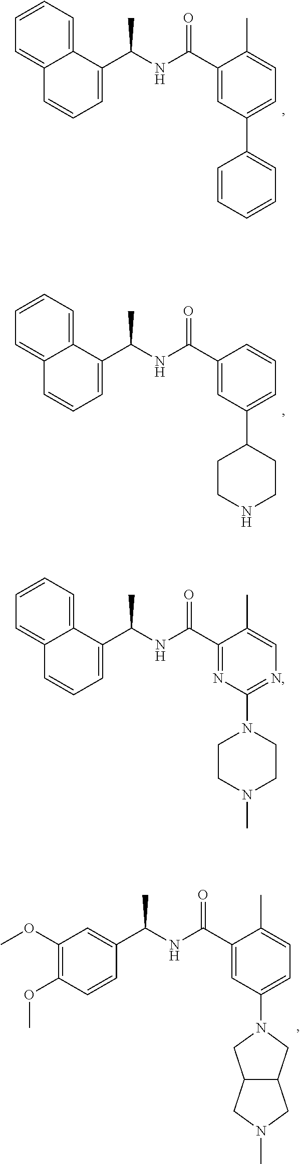 [SMILES: Cc1ccc(-c2ccccc2)cc1C(=O)N[C@H](C)c1cccc2ccccc12 ; C[C@@H](NC(=O)c1cccc(C2CCNCC2)c1)c1cccc2ccccc12 ; Cc1cnc(N2CCN(C)CC2)nc1C(=O)N[C@H](C)c1cccc2ccccc12 ; COc1ccc([C@@H](C)NC(=O)c2cc(N3CC4CN(C)CC4C3)ccc2C)cc1OC]

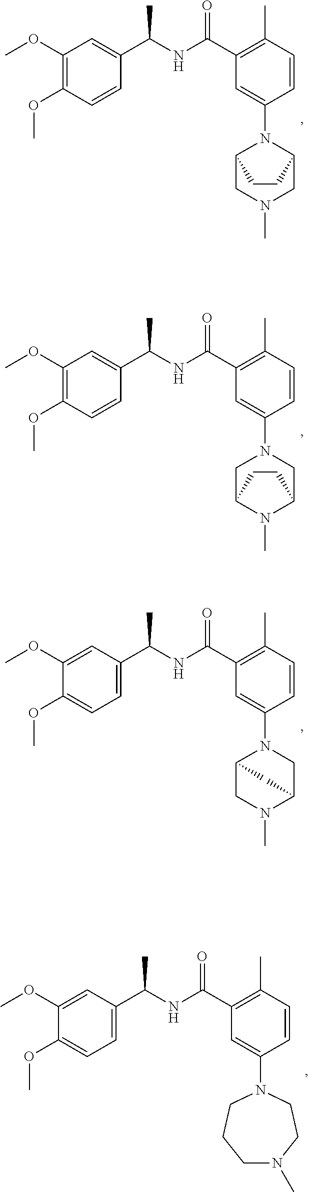 [SMILES: COc1ccc([C@@H](C)NC(=O)c2cc(N3[C@@H]4CC[C@H]3CN(C)C4)ccc2C)cc1OC ; COc1ccc([C@@H](C)NC(=O)c2cc(N3C[C@H]4CC[C@@H](C3)N4C)ccc2C)cc1OC ; COc1ccc([C@@H](C)NC(=O)c2cc(N3C[C@@H]4C[C@H]3CN4C)ccc2C)cc1OC ; COc1ccc([C@@H](C)NC(=O)c2cc(N3CCCN(C)CC3)ccc2C)cc1OC]

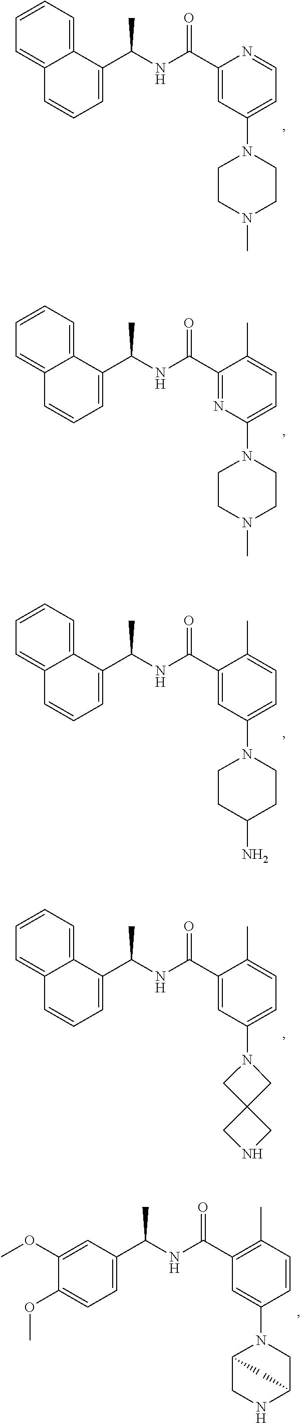 [SMILES: C[C@@H](NC(=O)c1cc(N2CCN(C)CC2)ccn1)c1cccc2ccccc12 ; Cc1ccc(N2CCN(C)CC2)nc1C(=O)N[C@H](C)c1cccc2ccccc12 ; Cc1ccc(N2CCC(N)CC2)cc1C(=O)N[C@H](C)c1cccc2ccccc12 ; Cc1ccc(N2CC3(CNC3)C2)cc1C(=O)N[C@H](C)c1cccc2ccccc12 ; COc1ccc([C@@H](C)NC(=O)c2cc(N3C[C@@H]4C[C@H]3CN4)ccc2C)cc1OC]

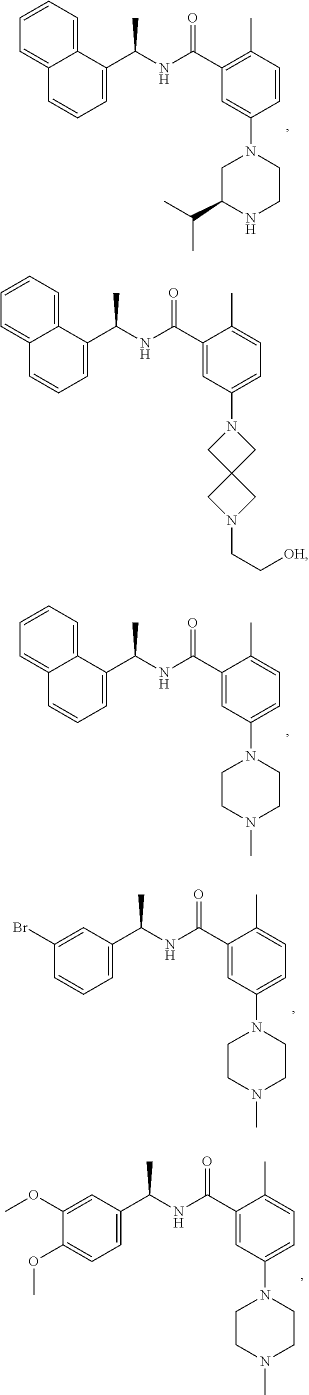

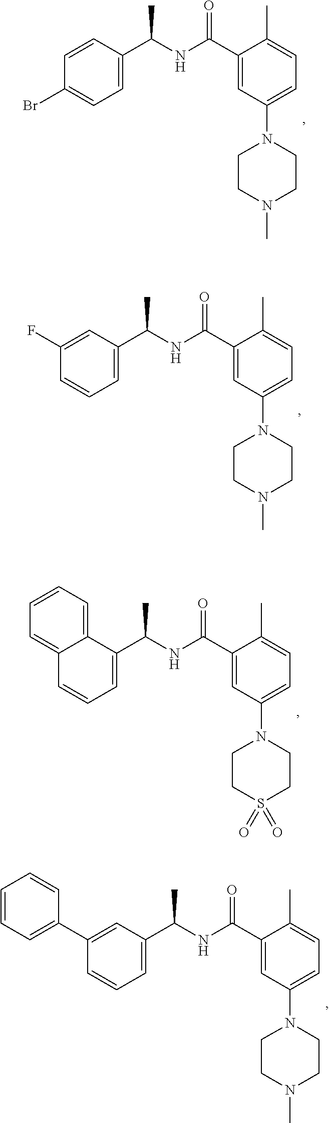 [SMILES: Cc1ccc(N2CCN(C)CC2)cc1C(=O)N[C@H](C)c1ccc(Br)cc1 ; Cc1ccc(N2CCN(C)CC2)cc1C(=O)N[C@H](C)c1cccc(F)c1 ; Cc1ccc(N2CCS(=O)(=O)CC2)cc1C(=O)N[C@H](C)c1cccc2ccccc12 ; Cc1ccc(N2CCN(C)CC2)cc1C(=O)N[C@H](C)c1cccc(-c2ccccc2)c1]

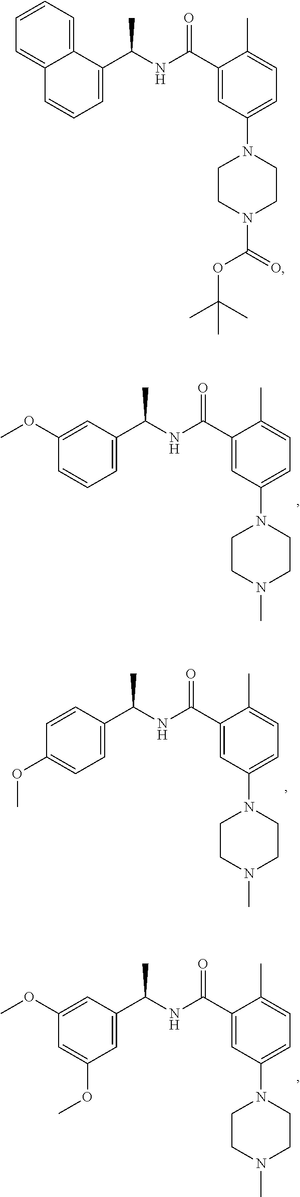 [SMILES: Cc1ccc(N2CCN(C(=O)OC(C)(C)C)CC2)cc1C(=O)N[C@H](C)c1cccc2ccccc12 ; COc1cccc([C@@H](C)NC(=O)c2cc(N3CCN(C)CC3)ccc2C)c1 ; COc1ccc([C@@H](C)NC(=O)c2cc(N3CCN(C)CC3)ccc2C)cc1 ; COc1cc(OC)cc([C@@H](C)NC(=O)c2cc(N3CCN(C)CC3)ccc2C)c1]

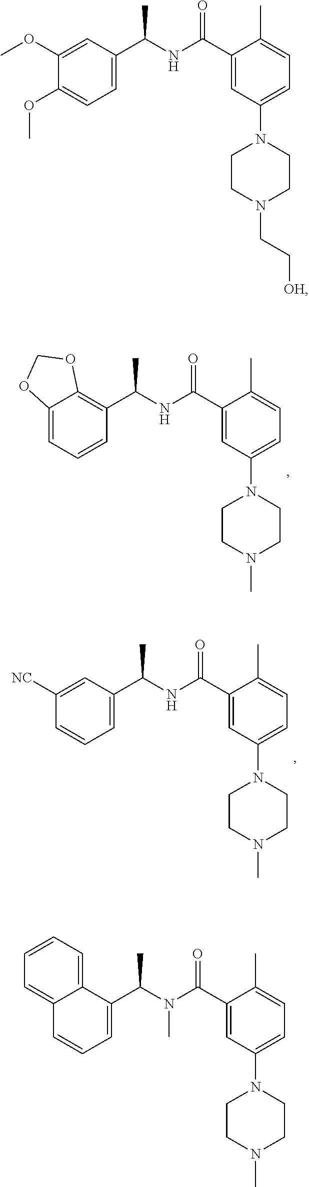 [SMILES: COc1ccc([C@@H](C)NC(=O)c2cc(N3CCN(CCO)CC3)ccc2C)cc1OC ; Cc1ccc(N2CCN(C)CC2)cc1C(=O)N[C@H](C)c1cccc2c1OCO2 ; Cc1ccc(N2CCN(C)CC2)cc1C(=O)N[C@H](C)c1cccc(C#N)c1 ; Cc1ccc(N2CCN(C)CC2)cc1C(=O)N(C)[C@H](C)c1cccc2ccccc12]

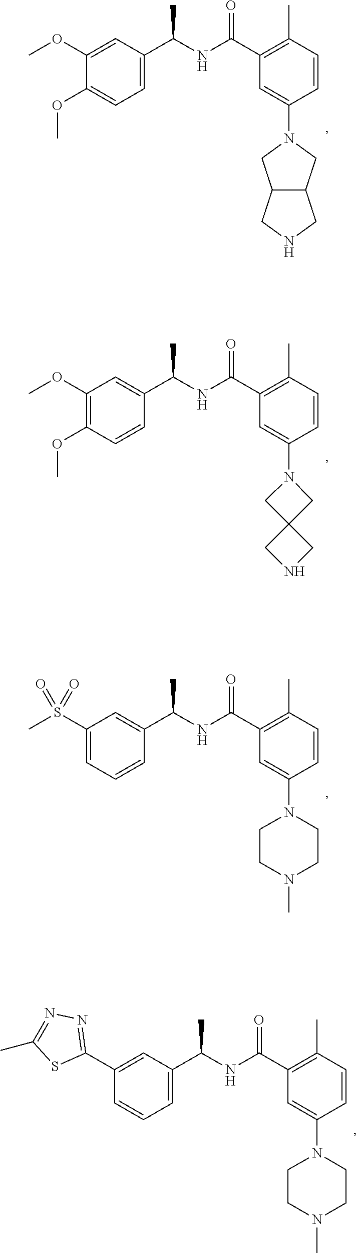 [SMILES: COc1ccc([C@@H](C)NC(=O)c2cc(N3CC4CNCC4C3)ccc2C)cc1OC ; COc1ccc([C@@H](C)NC(=O)c2cc(N3CC4(CNC4)C3)ccc2C)cc1OC ; Cc1ccc(N2CCN(C)CC2)cc1C(=O)N[C@H](C)c1cccc(S(C)(=O)=O)c1 ; Cc1nnc(-c2cccc([C@@H](C)NC(=O)c3cc(N4CCN(C)CC4)ccc3C)c2)s1]

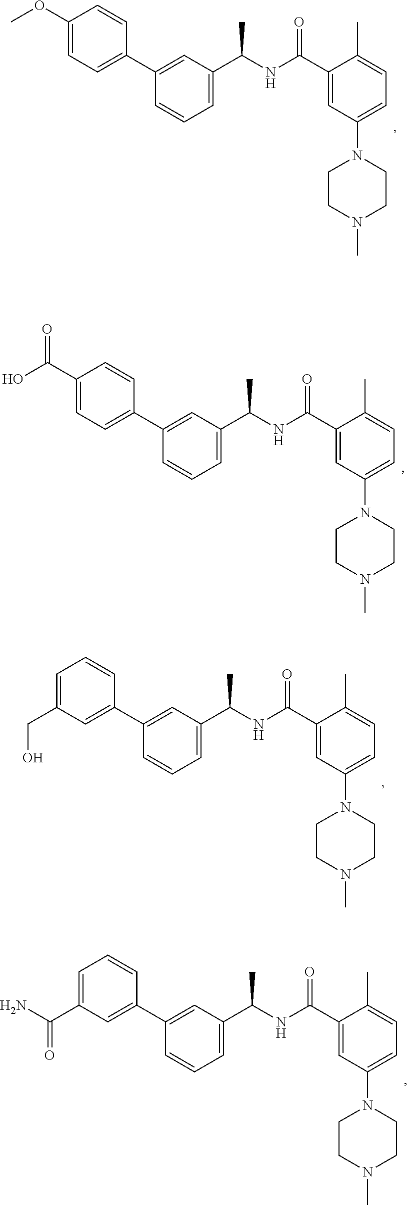 [SMILES: COc1ccc(-c2cccc([C@@H](C)NC(=O)c3cc(N4CCN(C)CC4)ccc3C)c2)cc1 ; Cc1ccc(N2CCN(C)CC2)cc1C(=O)N[C@H](C)c1cccc(-c2ccc(C(=O)O)cc2)c1 ; Cc1ccc(N2CCN(C)CC2)cc1C(=O)N[C@H](C)c1cccc(-c2cccc(CO)c2)c1 ; Cc1ccc(N2CCN(C)CC2)cc1C(=O)N[C@H](C)c1cccc(-c2cccc(C(N)=O)c2)c1]

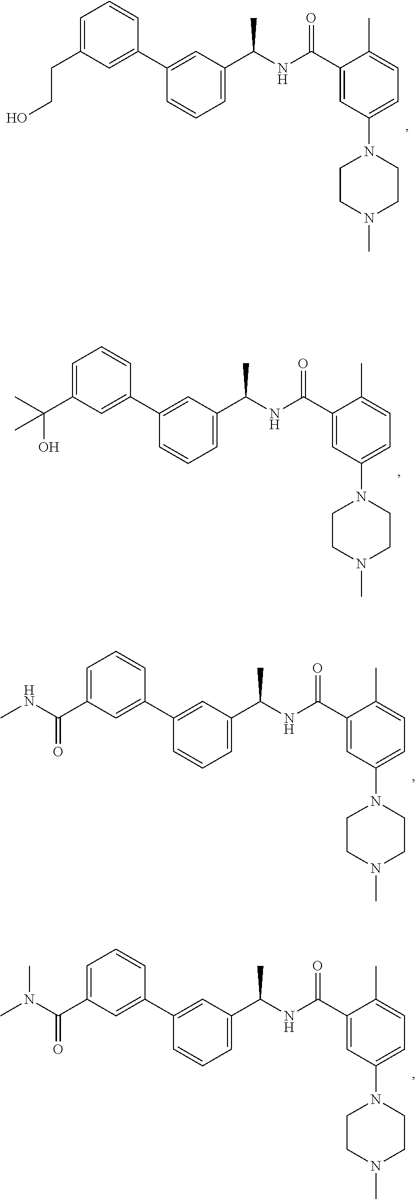 [SMILES: Cc1ccc(N2CCN(C)CC2)cc1C(=O)N[C@H](C)c1cccc(-c2cccc(CCO)c2)c1 ; Cc1ccc(N2CCN(C)CC2)cc1C(=O)N[C@H](C)c1cccc(-c2cccc(C(C)(C)O)c2)c1 ; CNC(=O)c1cccc(-c2cccc([C@@H](C)NC(=O)c3cc(N4CCN(C)CC4)ccc3C)c2)c1 ; Cc1ccc(N2CCN(C)CC2)cc1C(=O)N[C@H](C)c1cccc(-c2cccc(C(=O)N(C)C)c2)c1]

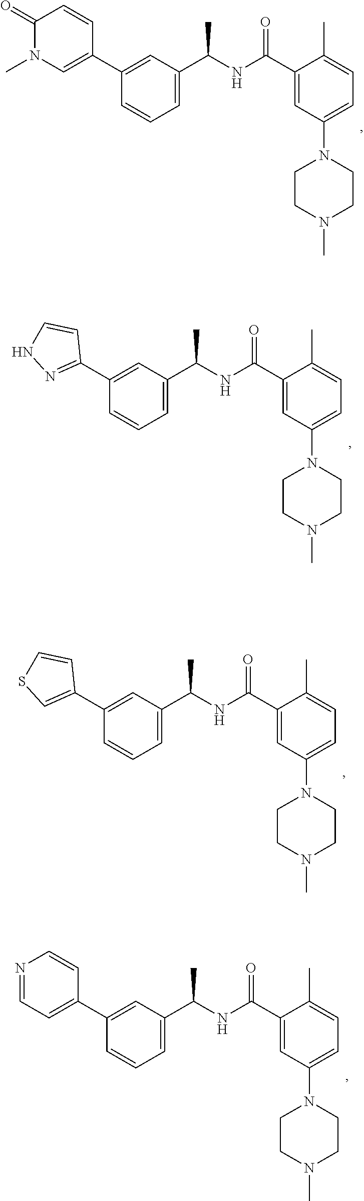 [SMILES: Cc1ccc(N2CCN(C)CC2)cc1C(=O)N[C@H](C)c1cccc(-c2ccc(=O)n(C)c2)c1 ; Cc1ccc(N2CCN(C)CC2)cc1C(=O)N[C@H](C)c1cccc(-c2cc[nH]n2)c1 ; Cc1ccc(N2CCN(C)CC2)cc1C(=O)N[C@H](C)c1cccc(-c2ccsc2)c1 ; Cc1ccc(N2CCN(C)CC2)cc1C(=O)N[C@H](C)c1cccc(-c2ccncc2)c1]

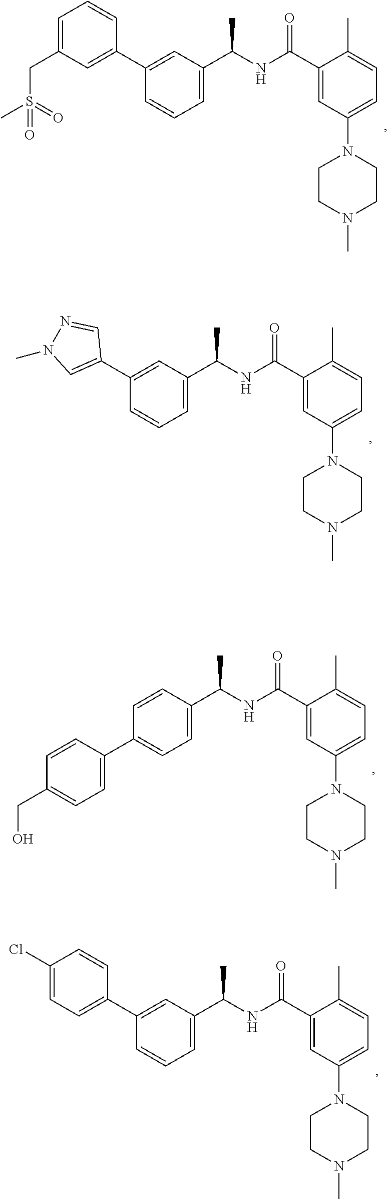 [SMILES: Cc1ccc(N2CCN(C)CC2)cc1C(=O)N[C@H](C)c1cccc(-c2cccc(CS(C)(=O)=O)c2)c1 ; Cc1ccc(N2CCN(C)CC2)cc1C(=O)N[C@H](C)c1cccc(-c2cnn(C)c2)c1 ; Cc1ccc(N2CCN(C)CC2)cc1C(=O)N[C@H](C)c1ccc(-c2ccc(CO)cc2)cc1 ; Cc1ccc(N2CCN(C)CC2)cc1C(=O)N[C@H](C)c1cccc(-c2ccc(Cl)cc2)c1]

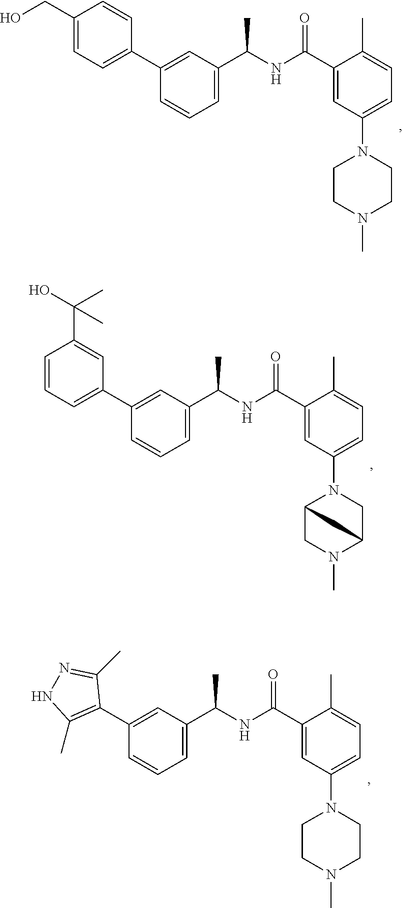 [SMILES: Cc1ccc(N2CCN(C)CC2)cc1C(=O)N[C@H](C)c1cccc(-c2ccc(CO)cc2)c1 ; Cc1ccc(N2C[C@H]3C[C@@H]2CN3C)cc1C(=O)N[C@H](C)c1cccc(-c2cccc(C(C)(C)O)c2)c1 ; Cc1ccc(N2CCN(C)CC2)cc1C(=O)N[C@H](C)c1cccc(-c2c(C)n[nH]c2C)c1]

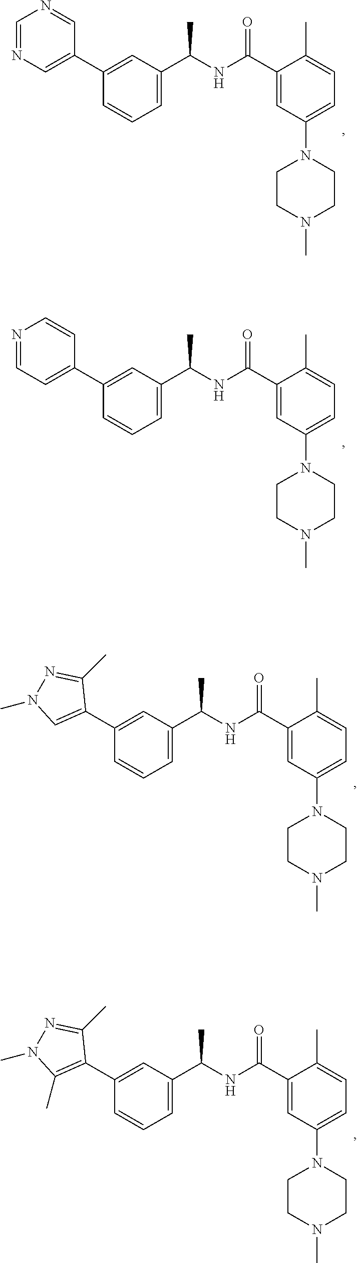 [SMILES: Cc1ccc(N2CCN(C)CC2)cc1C(=O)N[C@H](C)c1cccc(-c2cncnc2)c1 ; Cc1ccc(N2CCN(C)CC2)cc1C(=O)N[C@H](C)c1cccc(-c2ccncc2)c1 ; Cc1ccc(N2CCN(C)CC2)cc1C(=O)N[C@H](C)c1cccc(-c2cn(C)nc2C)c1 ; Cc1ccc(N2CCN(C)CC2)cc1C(=O)N[C@H](C)c1cccc(-c2c(C)nn(C)c2C)c1]

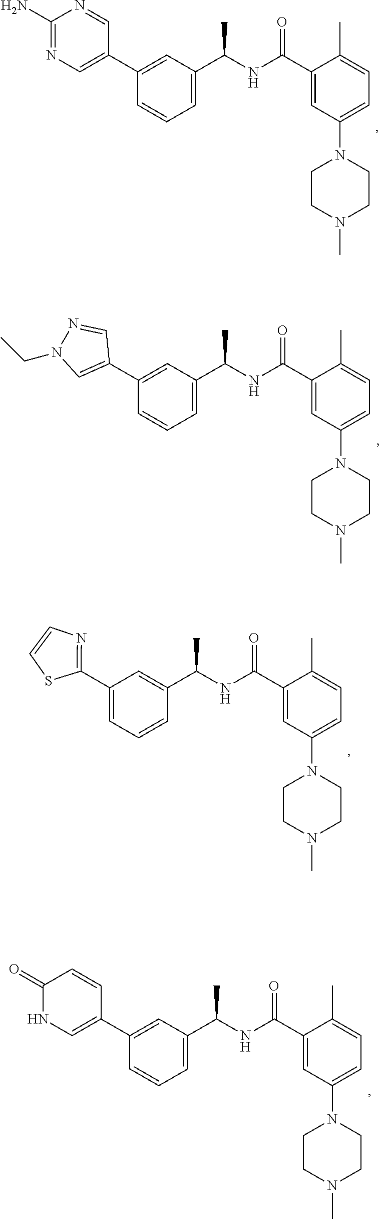 [SMILES: Cc1ccc(N2CCN(C)CC2)cc1C(=O)N[C@H](C)c1cccc(-c2cnc(N)nc2)c1 ; CCn1cc(-c2cccc([C@@H](C)NC(=O)c3cc(N4CCN(C)CC4)ccc3C)c2)cn1 ; Cc1ccc(N2CCN(C)CC2)cc1C(=O)N[C@H](C)c1cccc(-c2nccs2)c1 ; Cc1ccc(N2CCN(C)CC2)cc1C(=O)N[C@H](C)c1cccc(-c2ccc(=O)[nH]c2)c1]

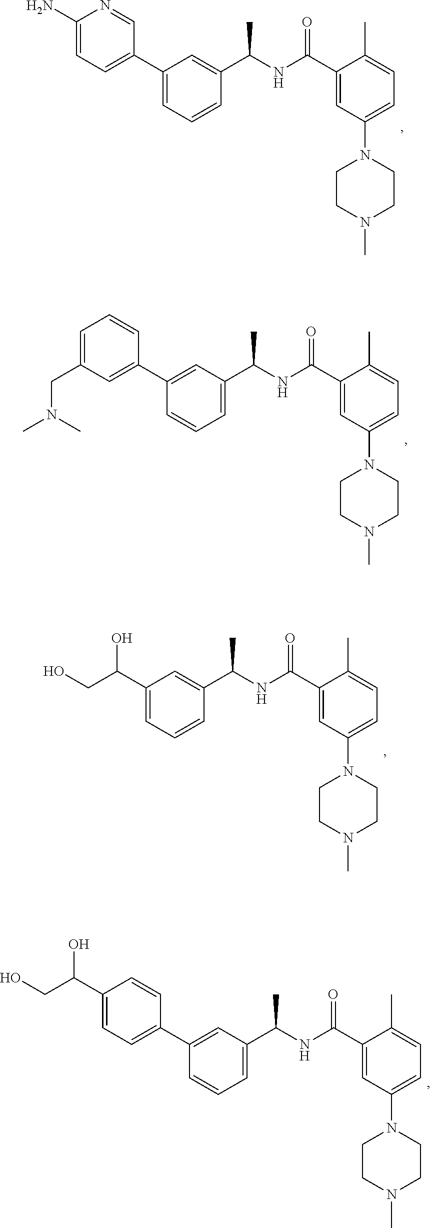 [SMILES: Cc1ccc(N2CCN(C)CC2)cc1C(=O)N[C@H](C)c1cccc(-c2ccc(N)nc2)c1 ; Cc1ccc(N2CCN(C)CC2)cc1C(=O)N[C@H](C)c1cccc(-c2cccc(CN(C)C)c2)c1 ; Cc1ccc(N2CCN(C)CC2)cc1C(=O)N[C@H](C)c1cccc(C(O)CO)c1 ; Cc1ccc(N2CCN(C)CC2)cc1C(=O)N[C@H](C)c1cccc(-c2ccc(C(O)CO)cc2)c1]

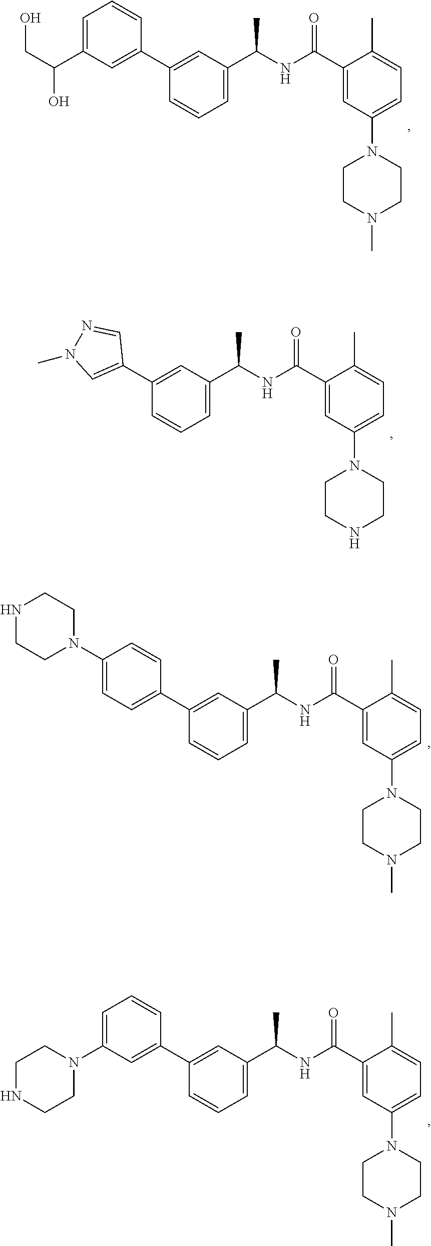 [SMILES: Cc1ccc(N2CCN(C)CC2)cc1C(=O)N[C@H](C)c1cccc(-c2cccc(C(O)CO)c2)c1 ; Cc1ccc(N2CCNCC2)cc1C(=O)N[C@H](C)c1cccc(-c2cnn(C)c2)c1 ; Cc1ccc(N2CCN(C)CC2)cc1C(=O)N[C@H](C)c1cccc(-c2ccc(N3CCNCC3)cc2)c1 ; Cc1ccc(N2CCN(C)CC2)cc1C(=O)N[C@H](C)c1cccc(-c2cccc(N3CCNCC3)c2)c1]

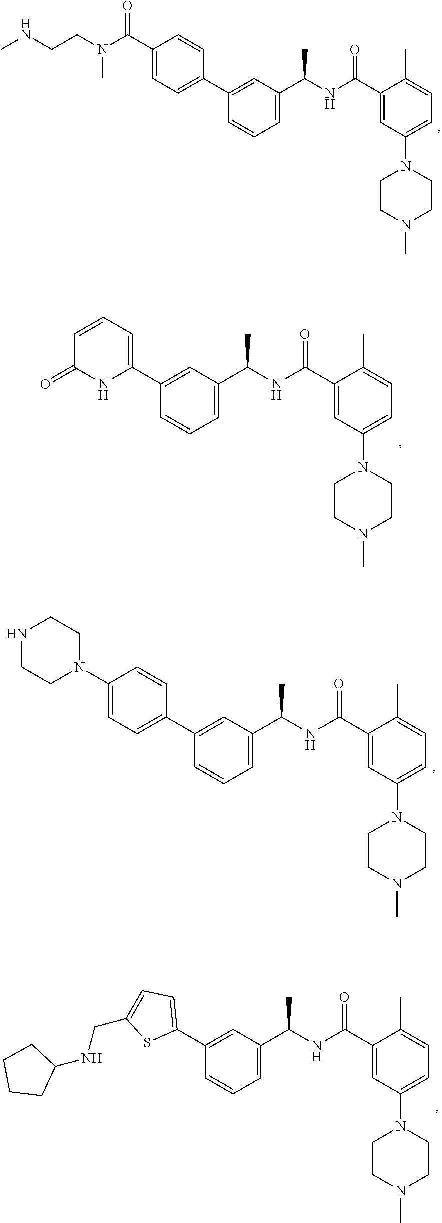 [SMILES: CNCCN(C)C(=O)c1ccc(-c2cccc([C@@H](C)NC(=O)c3cc(N4CCN(C)CC4)ccc3C)c2)cc1 ; Cc1ccc(N2CCN(C)CC2)cc1C(=O)N[C@H](C)c1cccc(-c2cccc(=O)[nH]2)c1 ; Cc1ccc(N2CCN(C)CC2)cc1C(=O)N[C@H](C)c1cccc(-c2ccc(N3CCNCC3)cc2)c1 ; Cc1ccc(N2CCN(C)CC2)cc1C(=O)N[C@H](C)c1cccc(-c2ccc(CNC3CCCC3)s2)c1]

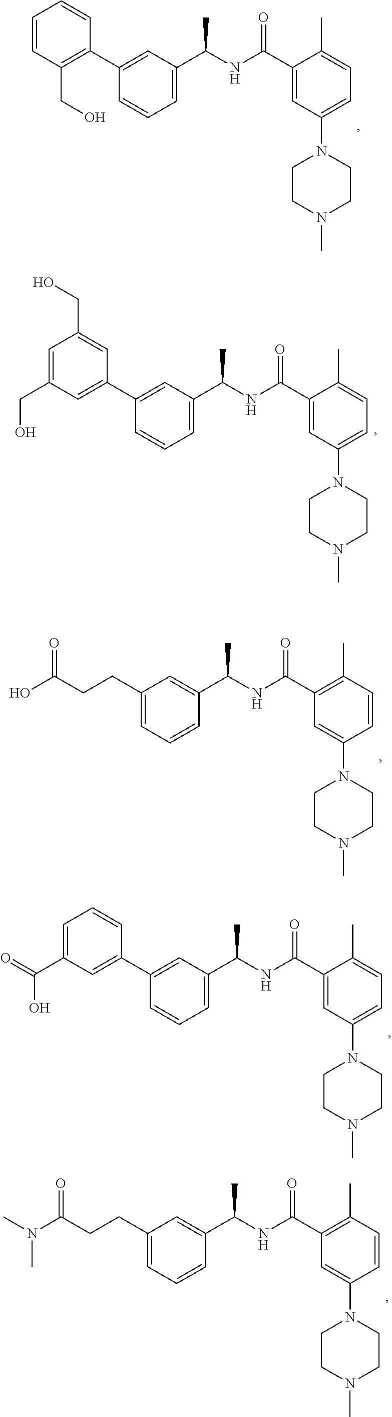

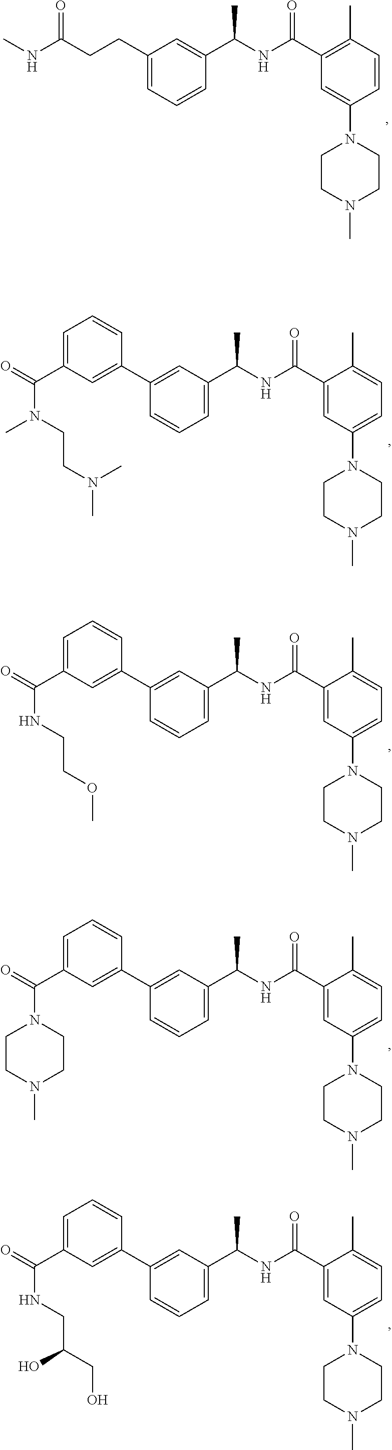

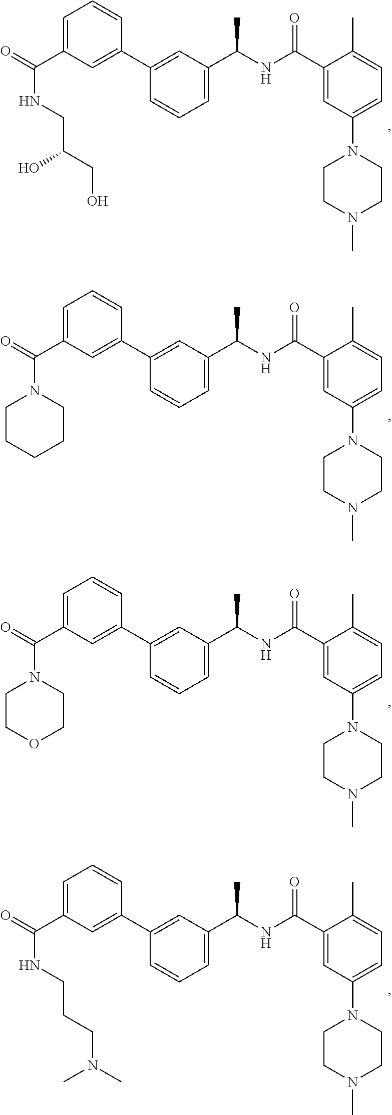 [SMILES: Cc1ccc(N2CCN(C)CC2)cc1C(=O)N[C@H](C)c1cccc(-c2cccc(C(=O)NC[C@@H](O)CO)c2)c1 ; Cc1ccc(N2CCN(C)CC2)cc1C(=O)N[C@H](C)c1cccc(-c2cccc(C(=O)N3CCCCC3)c2)c1 ; Cc1ccc(N2CCN(C)CC2)cc1C(=O)N[C@H](C)c1cccc(-c2cccc(C(=O)N3CCOCC3)c2)c1 ; Cc1ccc(N2CCN(C)CC2)cc1C(=O)N[C@H](C)c1cccc(-c2cccc(C(=O)NCCCN(C)C)c2)c1]

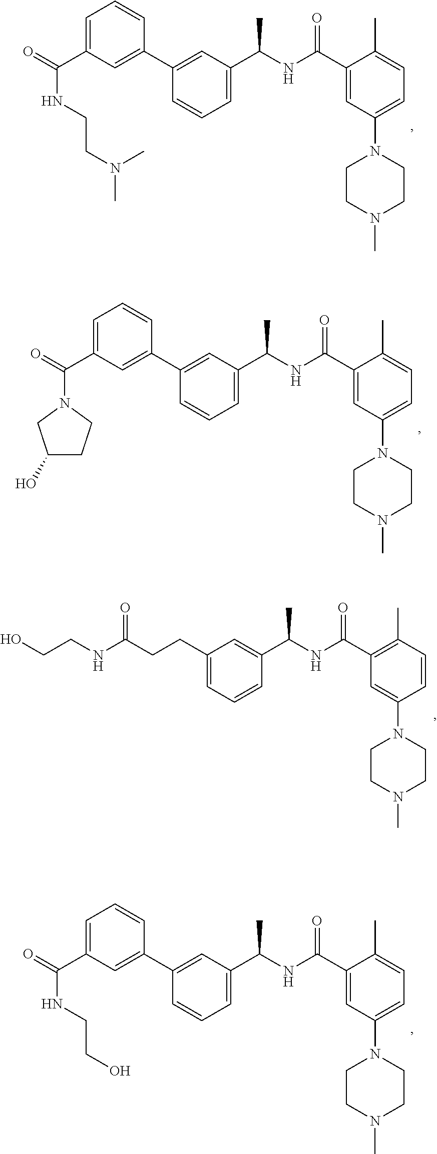 [SMILES: Cc1ccc(N2CCN(C)CC2)cc1C(=O)N[C@H](C)c1cccc(-c2cccc(C(=O)NCCN(C)C)c2)c1 ; Cc1ccc(N2CCN(C)CC2)cc1C(=O)N[C@H](C)c1cccc(-c2cccc(C(=O)N3CC[C@H](O)C3)c2)c1 ; Cc1ccc(N2CCN(C)CC2)cc1C(=O)N[C@H](C)c1cccc(CCC(=O)NCCO)c1 ; Cc1ccc(N2CCN(C)CC2)cc1C(=O)N[C@H](C)c1cccc(-c2cccc(C(=O)NCCO)c2)c1]

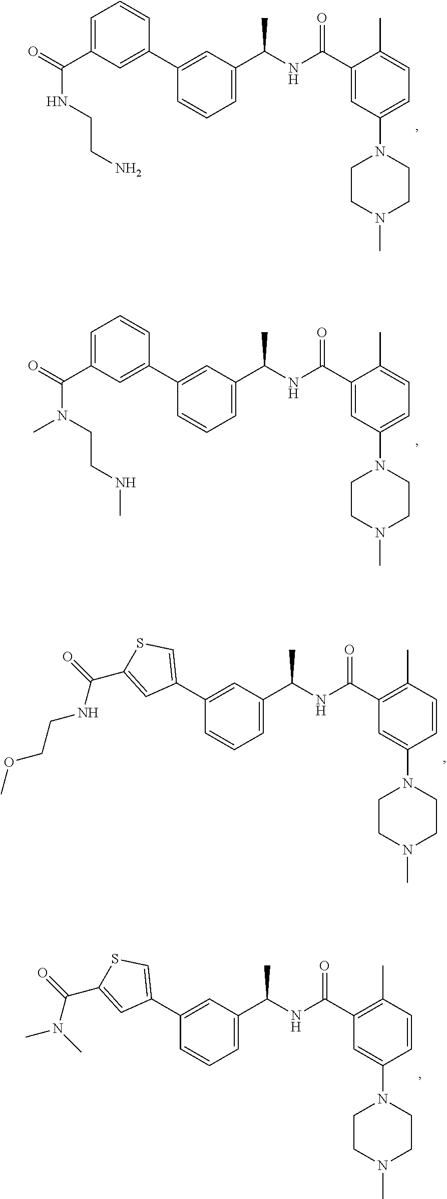 [SMILES: Cc1ccc(N2CCN(C)CC2)cc1C(=O)N[C@H](C)c1cccc(-c2cccc(C(=O)NCCN)c2)c1 ; CNCCN(C)C(=O)c1cccc(-c2cccc([C@@H](C)NC(=O)c3cc(N4CCN(C)CC4)ccc3C)c2)c1 ; COCCNC(=O)c1cc(-c2cccc([C@@H](C)NC(=O)c3cc(N4CCN(C)CC4)ccc3C)c2)cs1 ; Cc1ccc(N2CCN(C)CC2)cc1C(=O)N[C@H](C)c1cccc(-c2csc(C(=O)N(C)C)c2)c1]

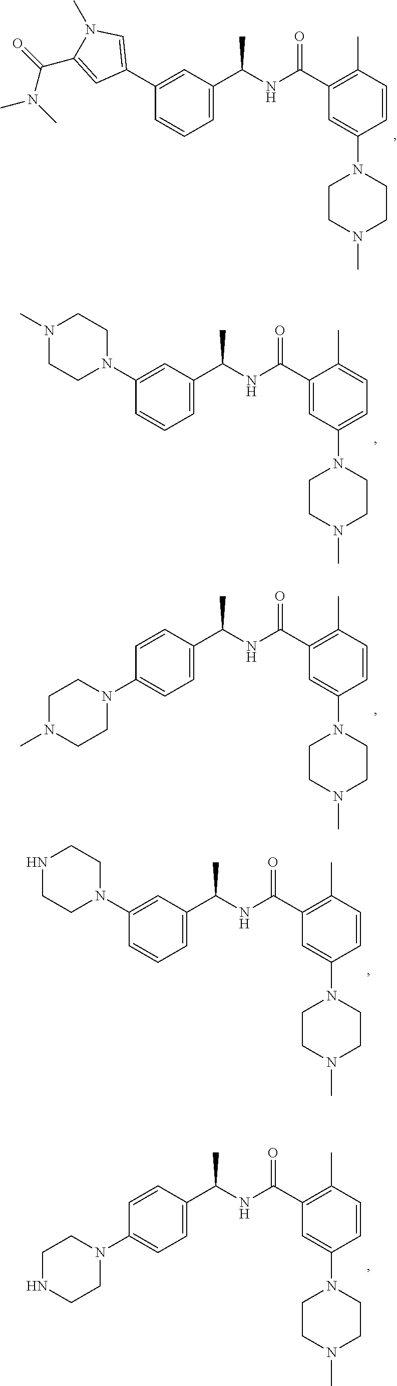

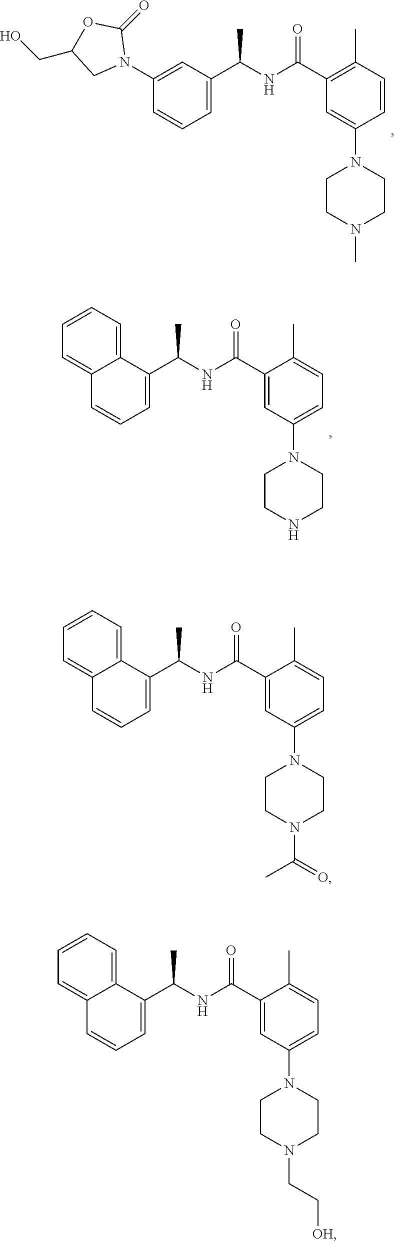 [SMILES: Cc1ccc(N2CCN(C)CC2)cc1C(=O)N[C@H](C)c1cccc(N2CC(CO)OC2=O)c1 ; Cc1ccc(N2CCNCC2)cc1C(=O)N[C@H](C)c1cccc2ccccc12 ; CC(=O)N1CCN(c2ccc(C)c(C(=O)N[C@H](C)c3cccc4ccccc34)c2)CC1 ; Cc1ccc(N2CCN(CCO)CC2)cc1C(=O)N[C@H](C)c1cccc2ccccc12]

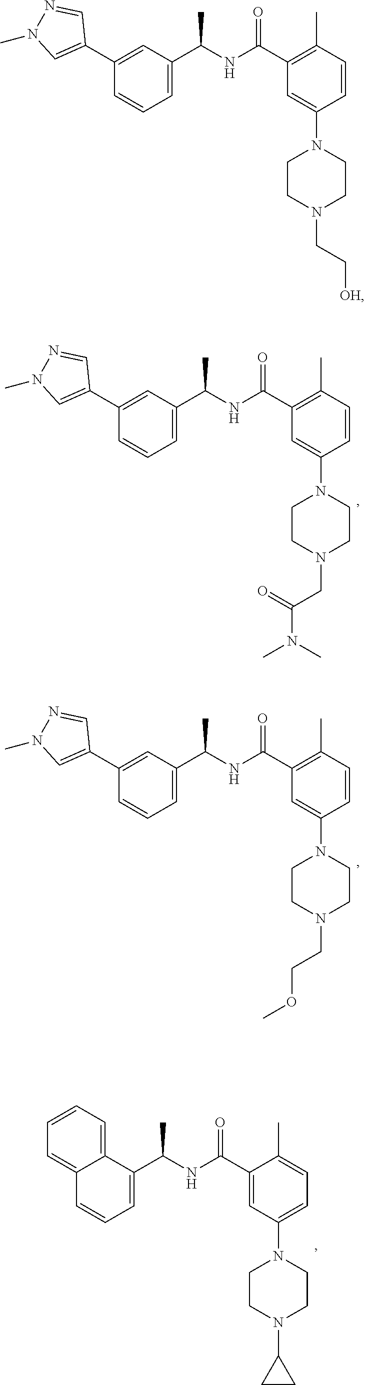 [SMILES: Cc1ccc(N2CCN(CCO)CC2)cc1C(=O)N[C@H](C)c1cccc(-c2cnn(C)c2)c1 ; Cc1ccc(N2CCN(CC(=O)N(C)C)CC2)cc1C(=O)N[C@H](C)c1cccc(-c2cnn(C)c2)c1 ; COCCN1CCN(c2ccc(C)c(C(=O)N[C@H](C)c3cccc(-c4cnn(C)c4)c3)c2)CC1 ; Cc1ccc(N2CCN(C3CC3)CC2)cc1C(=O)N[C@H](C)c1cccc2ccccc12]

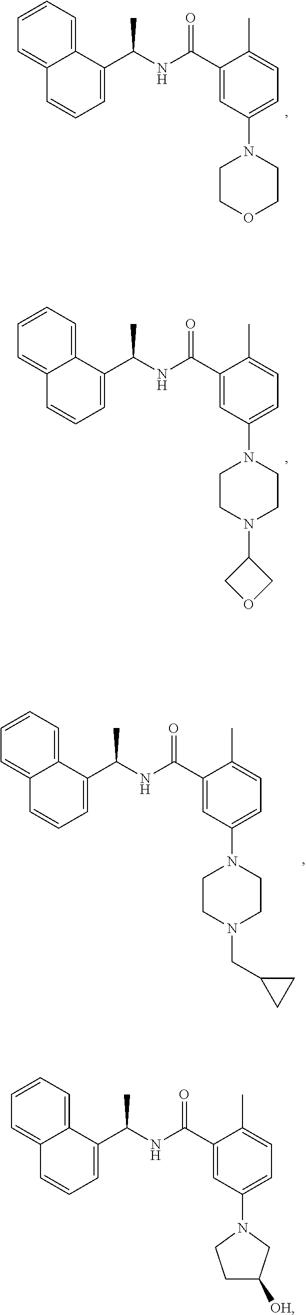 [SMILES: Cc1ccc(N2CCOCC2)cc1C(=O)N[C@H](C)c1cccc2ccccc12 ; Cc1ccc(N2CCN(C3COC3)CC2)cc1C(=O)N[C@H](C)c1cccc2ccccc12 ; Cc1ccc(N2CCN(CC3CC3)CC2)cc1C(=O)N[C@H](C)c1cccc2ccccc12 ; Cc1ccc(N2CC[C@H](O)C2)cc1C(=O)N[C@H](C)c1cccc2ccccc12]

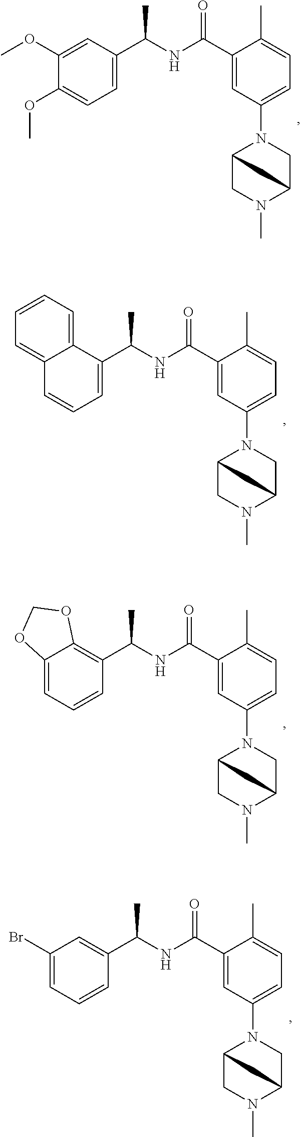 [SMILES: COc1ccc([C@@H](C)NC(=O)c2cc(N3C[C@H]4C[C@@H]3CN4C)ccc2C)cc1OC ; Cc1ccc(N2C[C@H]3C[C@@H]2CN3C)cc1C(=O)N[C@H](C)c1cccc2ccccc12 ; Cc1ccc(N2C[C@H]3C[C@@H]2CN3C)cc1C(=O)N[C@H](C)c1cccc2c1OCO2 ; Cc1ccc(N2C[C@H]3C[C@@H]2CN3C)cc1C(=O)N[C@H](C)c1cccc(Br)c1]

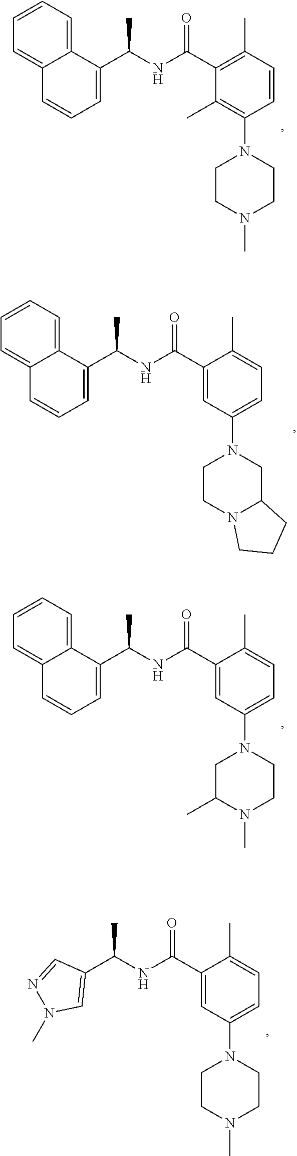 [SMILES: Cc1ccc(N2CCN(C)CC2)c(C)c1C(=O)N[C@H](C)c1cccc2ccccc12 ; Cc1ccc(N2CCN3CCCC3C2)cc1C(=O)N[C@H](C)c1cccc2ccccc12 ; Cc1ccc(N2CCN(C)C(C)C2)cc1C(=O)N[C@H](C)c1cccc2ccccc12 ; Cc1ccc(N2CCN(C)CC2)cc1C(=O)N[C@H](C)c1cnn(C)c1]

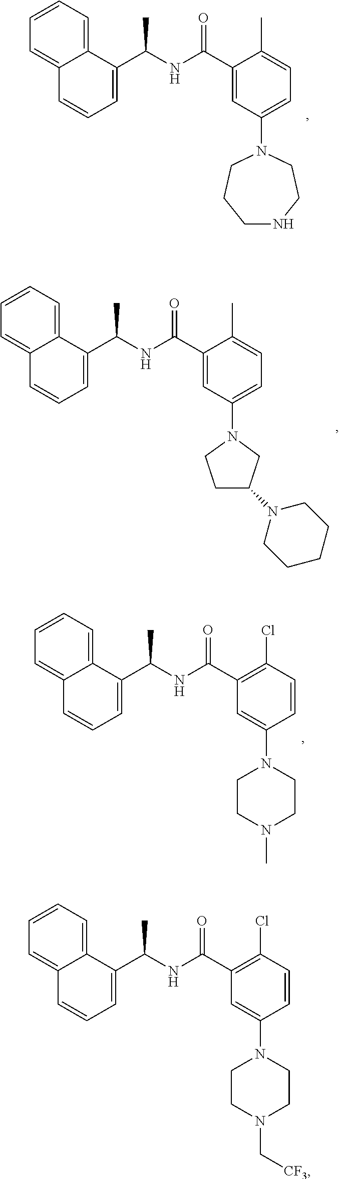 [SMILES: Cc1ccc(N2CCCNCC2)cc1C(=O)N[C@H](C)c1cccc2ccccc12 ; Cc1ccc(N2CC[C@@H](N3CCCCC3)C2)cc1C(=O)N[C@H](C)c1cccc2ccccc12 ; C[C@@H](NC(=O)c1cc(N2CCN(C)CC2)ccc1Cl)c1cccc2ccccc12 ; C[C@@H](NC(=O)c1cc(N2CCN(CC(F)(F)F)CC2)ccc1Cl)c1cccc2ccccc12]

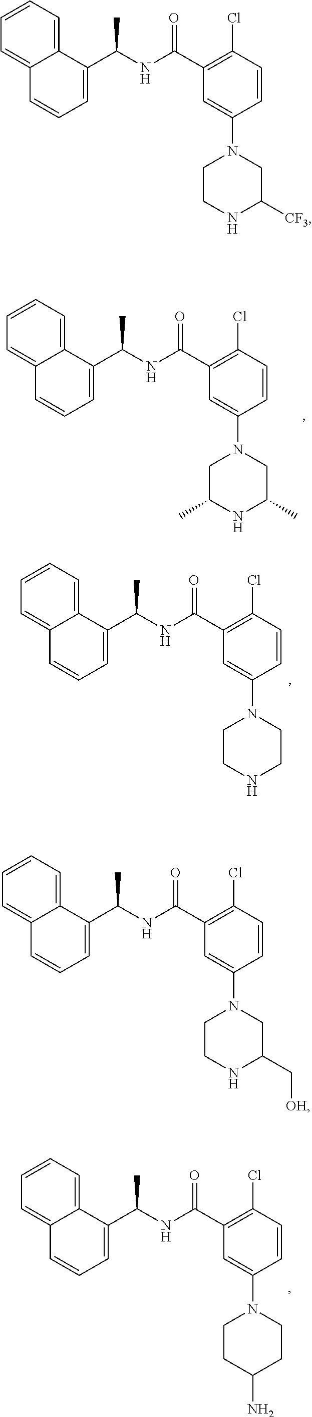 [SMILES: C[C@@H](NC(=O)c1cc(N2CCNC(C(F)(F)F)C2)ccc1Cl)c1cccc2ccccc12 ; C[C@@H]1CN(c2ccc(Cl)c(C(=O)N[C@H](C)c3cccc4ccccc34)c2)C[C@H](C)N1 ; C[C@@H](NC(=O)c1cc(N2CCNCC2)ccc1Cl)c1cccc2ccccc12 ; C[C@@H](NC(=O)c1cc(N2CCNC(CO)C2)ccc1Cl)c1cccc2ccccc12 ; C[C@@H](NC(=O)c1cc(N2CCC(N)CC2)ccc1Cl)c1cccc2ccccc12]

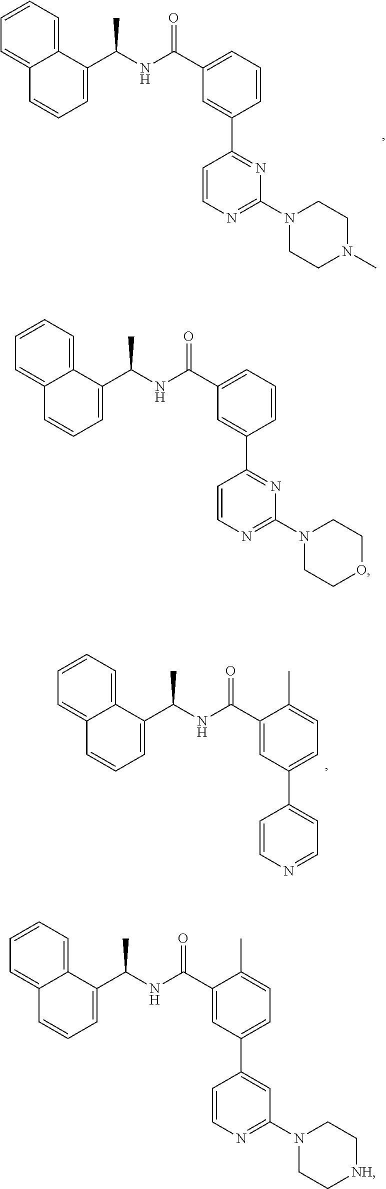 [SMILES: C[C@@H](NC(=O)c1cccc(-c2ccnc(N3CCN(C)CC3)n2)c1)c1cccc2ccccc12 ; C[C@@H](NC(=O)c1cccc(-c2ccnc(N3CCOCC3)n2)c1)c1cccc2ccccc12 ; Cc1ccc(-c2ccncc2)cc1C(=O)N[C@H](C)c1cccc2ccccc12 ; Cc1ccc(-c2ccnc(N3CCNCC3)c2)cc1C(=O)N[C@H](C)c1cccc2ccccc12]

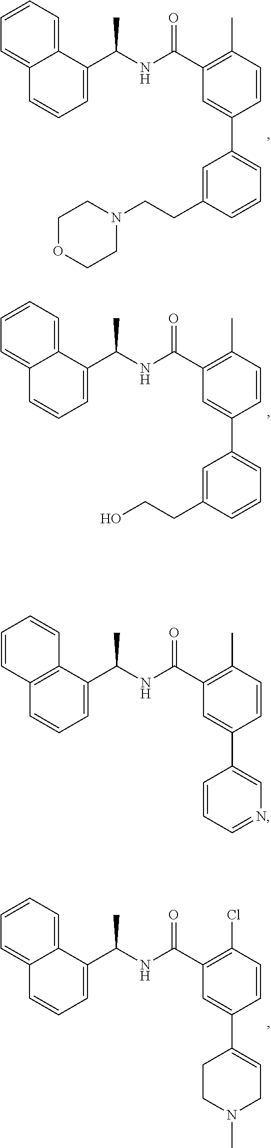 [SMILES: Cc1ccc(-c2cccc(CCN3CCOCC3)c2)cc1C(=O)N[C@H](C)c1cccc2ccccc12 ; Cc1ccc(-c2cccc(CCO)c2)cc1C(=O)N[C@H](C)c1cccc2ccccc12 ; Cc1ccc(-c2cccnc2)cc1C(=O)N[C@H](C)c1cccc2ccccc12 ; C[C@@H](NC(=O)c1cc(C2=CCN(C)CC2)ccc1Cl)c1cccc2ccccc12]

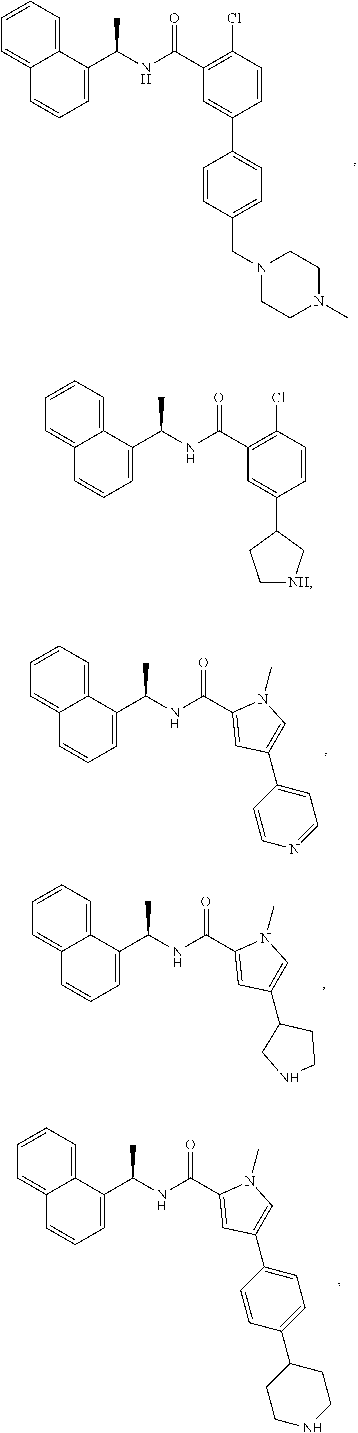 [SMILES: C[C@@H](NC(=O)c1cc(-c2ccc(CN3CCN(C)CC3)cc2)ccc1Cl)c1cccc2ccccc12 ; C[C@@H](NC(=O)c1cc(C2CCNC2)ccc1Cl)c1cccc2ccccc12 ; C[C@@H](NC(=O)c1cc(-c2ccncc2)cn1C)c1cccc2ccccc12 ; C[C@@H](NC(=O)c1cc(C2CCNC2)cn1C)c1cccc2ccccc12 ; C[C@@H](NC(=O)c1cc(-c2ccc(C3CCNCC3)cc2)cn1C)c1cccc2ccccc12]

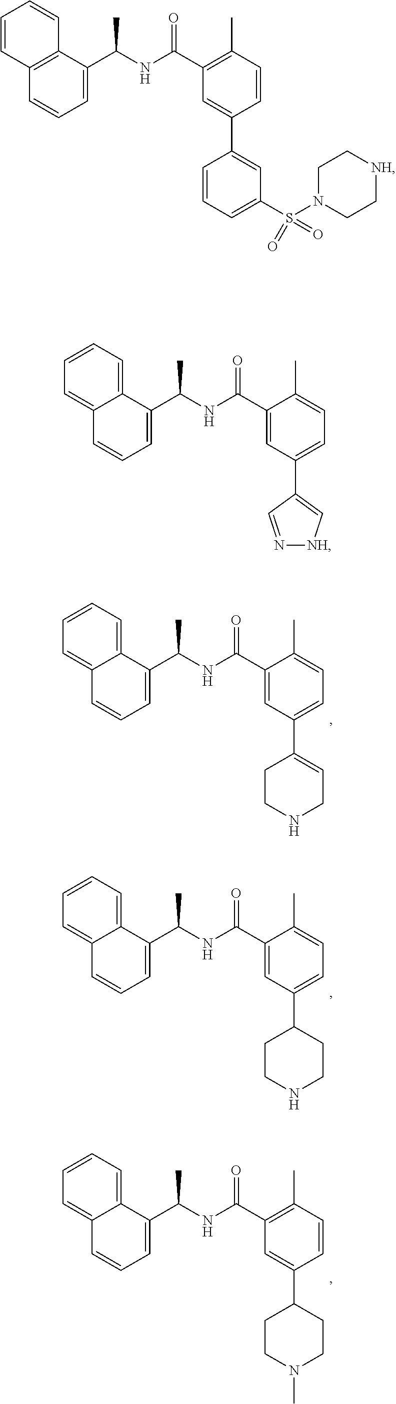 [SMILES: Cc1ccc(-c2cccc(S(=O)(=O)N3CCNCC3)c2)cc1C(=O)N[C@H](C)c1cccc2ccccc12 ; Cc1ccc(-c2cn[nH]c2)cc1C(=O)N[C@H](C)c1cccc2ccccc12 ; Cc1ccc(C2=CCNCC2)cc1C(=O)N[C@H](C)c1cccc2ccccc12 ; Cc1ccc(C2CCNCC2)cc1C(=O)N[C@H](C)c1cccc2ccccc12 ; Cc1ccc(C2CCN(C)CC2)cc1C(=O)N[C@H](C)c1cccc2ccccc12]

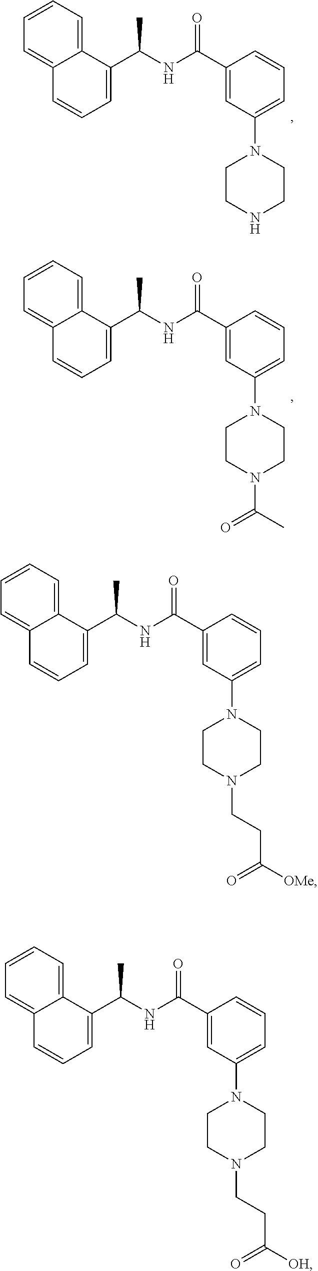 [SMILES: C[C@@H](NC(=O)c1cccc(N2CCNCC2)c1)c1cccc2ccccc12 ; CC(=O)N1CCN(c2cccc(C(=O)N[C@H](C)c3cccc4ccccc34)c2)CC1 ; COC(=O)CCN1CCN(c2cccc(C(=O)N[C@H](C)c3cccc4ccccc34)c2)CC1 ; C[C@@H](NC(=O)c1cccc(N2CCN(CCC(=O)O)CC2)c1)c1cccc2ccccc12]

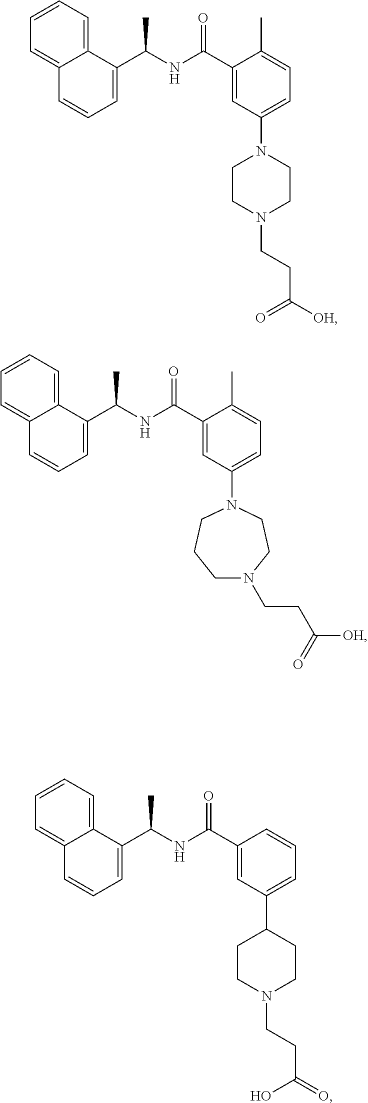 [SMILES: Cc1ccc(N2CCN(CCC(=O)O)CC2)cc1C(=O)N[C@H](C)c1cccc2ccccc12 ; Cc1ccc(N2CCCN(CCC(=O)O)CC2)cc1C(=O)N[C@H](C)c1cccc2ccccc12 ; C[C@@H](NC(=O)c1cccc(C2CCN(CCC(=O)O)CC2)c1)c1cccc2ccccc12]

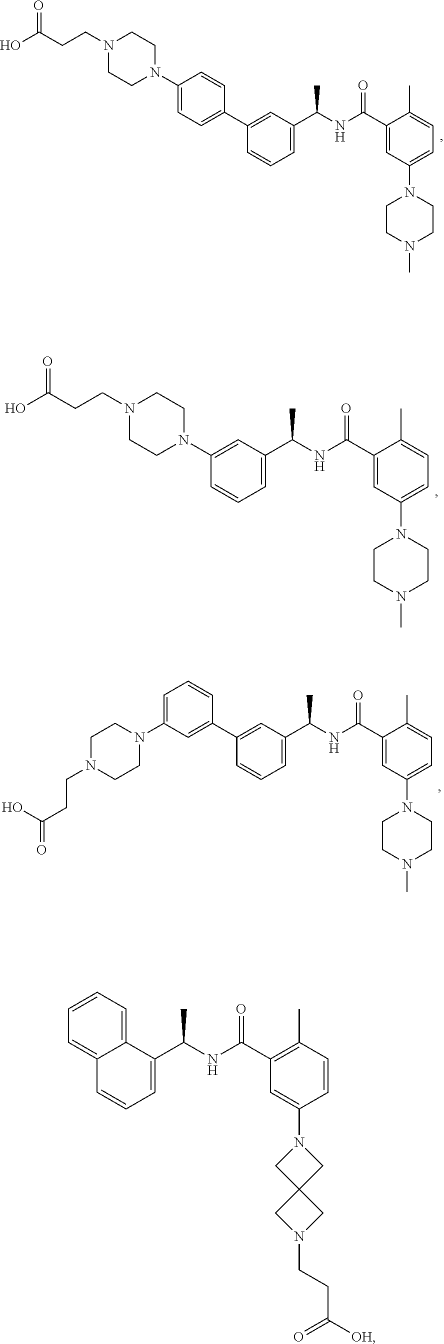 [SMILES: Cc1ccc(N2CCN(C)CC2)cc1C(=O)N[C@H](C)c1cccc(-c2ccc(N3CCN(CCC(=O)O)CC3)cc2)c1 ; Cc1ccc(N2CCN(C)CC2)cc1C(=O)N[C@H](C)c1cccc(N2CCN(CCC(=O)O)CC2)c1 ; Cc1ccc(N2CCN(C)CC2)cc1C(=O)N[C@H](C)c1cccc(-c2cccc(N3CCN(CCC(=O)O)CC3)c2)c1 ; Cc1ccc(N2CC3(CN(CCC(=O)O)C3)C2)cc1C(=O)N[C@H](C)c1cccc2ccccc12]

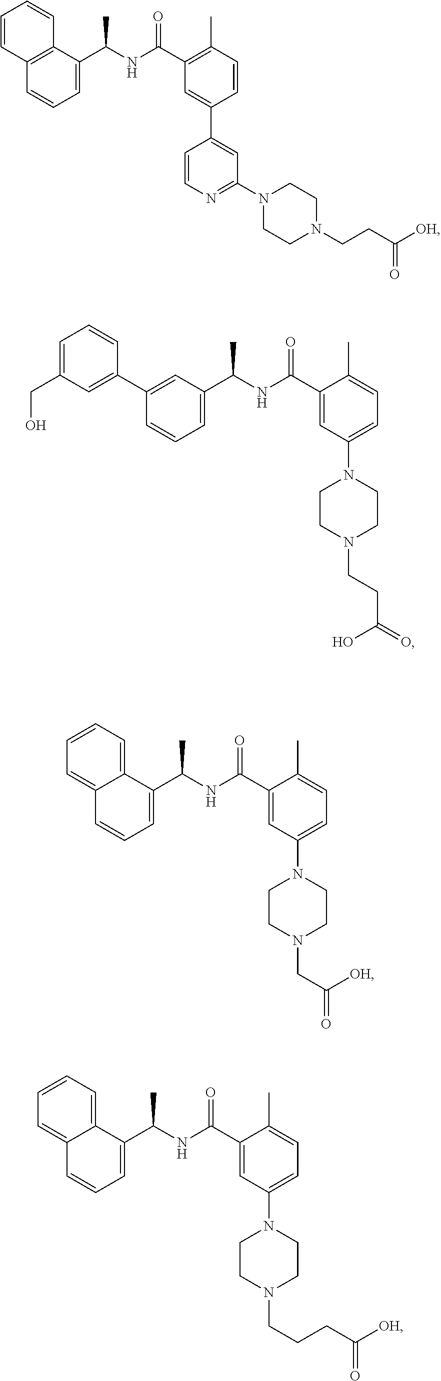 [SMILES: Cc1ccc(-c2ccnc(N3CCN(CCC(=O)O)CC3)c2)cc1C(=O)N[C@H](C)c1cccc2ccccc12 ; Cc1ccc(N2CCN(CCC(=O)O)CC2)cc1C(=O)N[C@H](C)c1cccc(-c2cccc(CO)c2)c1 ; Cc1ccc(N2CCN(CC(=O)O)CC2)cc1C(=O)N[C@H](C)c1cccc2ccccc12 ; Cc1ccc(N2CCN(CCCC(=O)O)CC2)cc1C(=O)N[C@H](C)c1cccc2ccccc12]

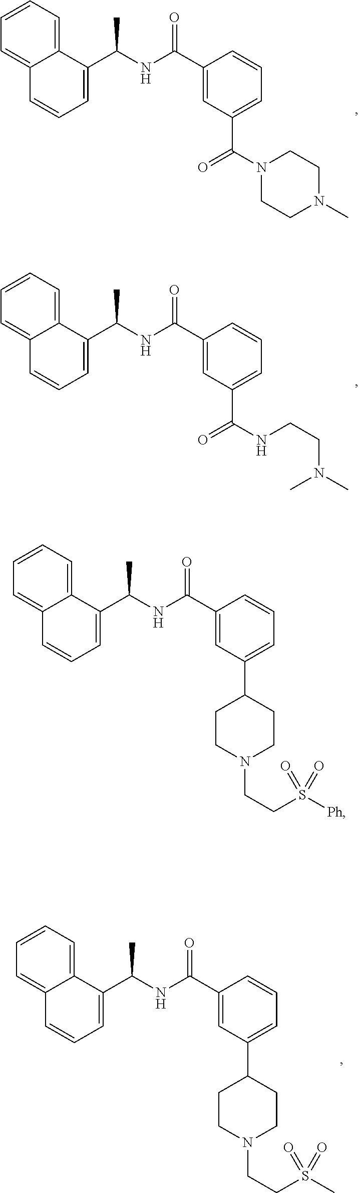 [SMILES: C[C@@H](NC(=O)c1cccc(C(=O)N2CCN(C)CC2)c1)c1cccc2ccccc12 ; C[C@@H](NC(=O)c1cccc(C(=O)NCCN(C)C)c1)c1cccc2ccccc12 ; C[C@@H](NC(=O)c1cccc(C2CCN(CCS(=O)(=O)c3ccccc3)CC2)c1)c1cccc2ccccc12 ; C[C@@H](NC(=O)c1cccc(C2CCN(CCS(C)(=O)=O)CC2)c1)c1cccc2ccccc12]

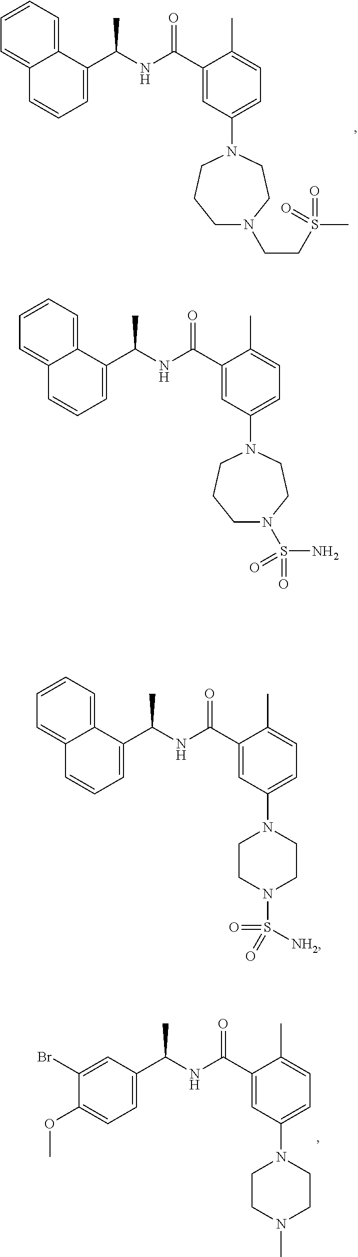 [SMILES: Cc1ccc(N2CCCN(CCS(C)(=O)=O)CC2)cc1C(=O)N[C@H](C)c1cccc2ccccc12 ; Cc1ccc(N2CCCN(S(N)(=O)=O)CC2)cc1C(=O)N[C@H](C)c1cccc2ccccc12 ; Cc1ccc(N2CCN(S(N)(=O)=O)CC2)cc1C(=O)N[C@H](C)c1cccc2ccccc12 ; COc1ccc([C@@H](C)NC(=O)c2cc(N3CCN(C)CC3)ccc2C)cc1Br]

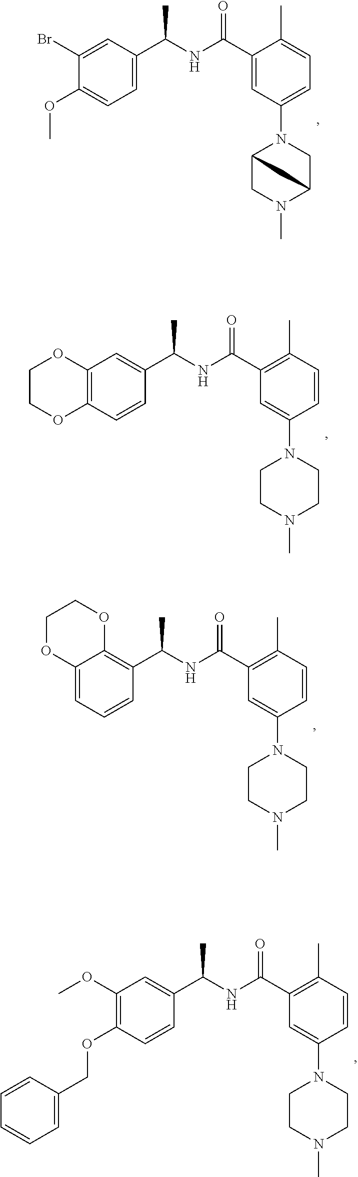 [SMILES: COc1ccc([C@@H](C)NC(=O)c2cc(N3C[C@H]4C[C@@H]3CN4C)ccc2C)cc1Br ; Cc1ccc(N2CCN(C)CC2)cc1C(=O)N[C@H](C)c1ccc2c(c1)OCCO2 ; Cc1ccc(N2CCN(C)CC2)cc1C(=O)N[C@H](C)c1cccc2c1OCCO2 ; COc1cc([C@@H](C)NC(=O)c2cc(N3CCN(C)CC3)ccc2C)ccc1OCc1ccccc1]

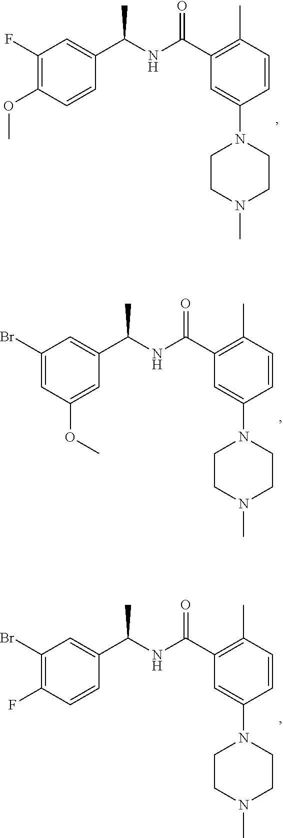 [SMILES: COc1ccc([C@@H](C)NC(=O)c2cc(N3CCN(C)CC3)ccc2C)cc1F ; COc1cc(Br)cc([C@@H](C)NC(=O)c2cc(N3CCN(C)CC3)ccc2C)c1 ; Cc1ccc(N2CCN(C)CC2)cc1C(=O)N[C@H](C)c1ccc(F)c(Br)c1]

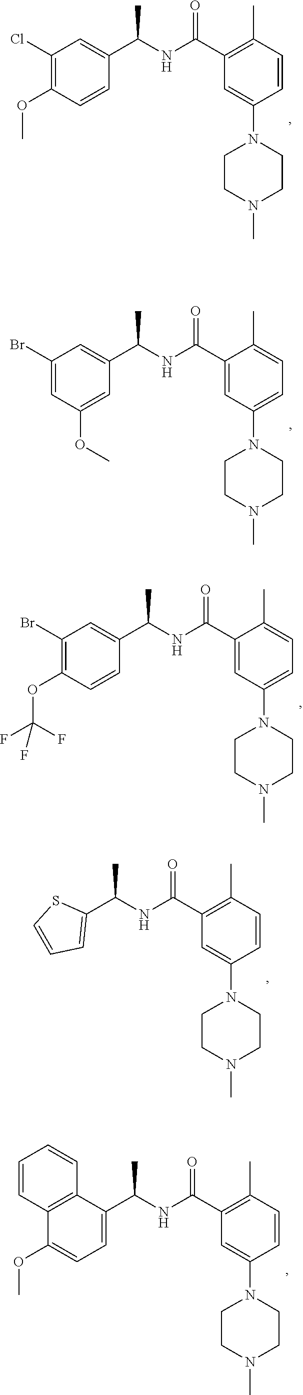

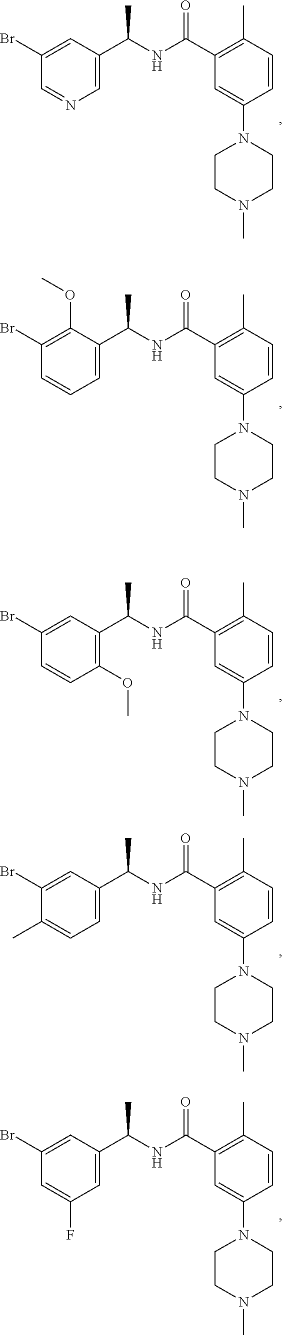

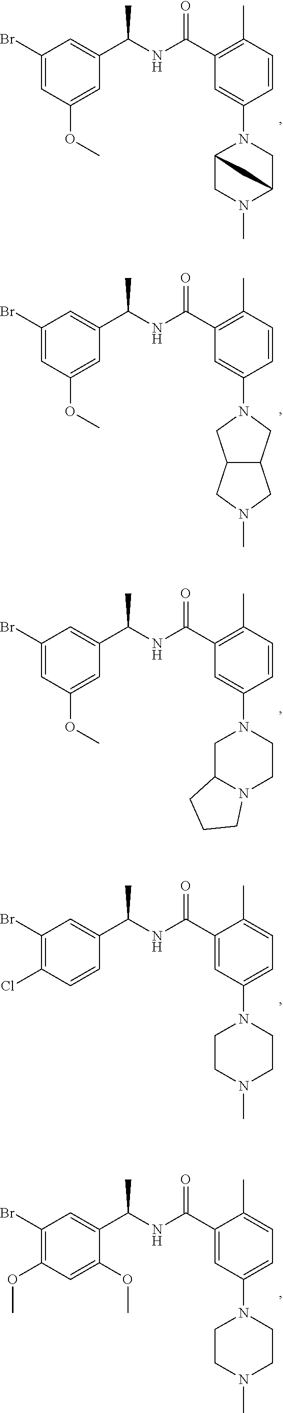

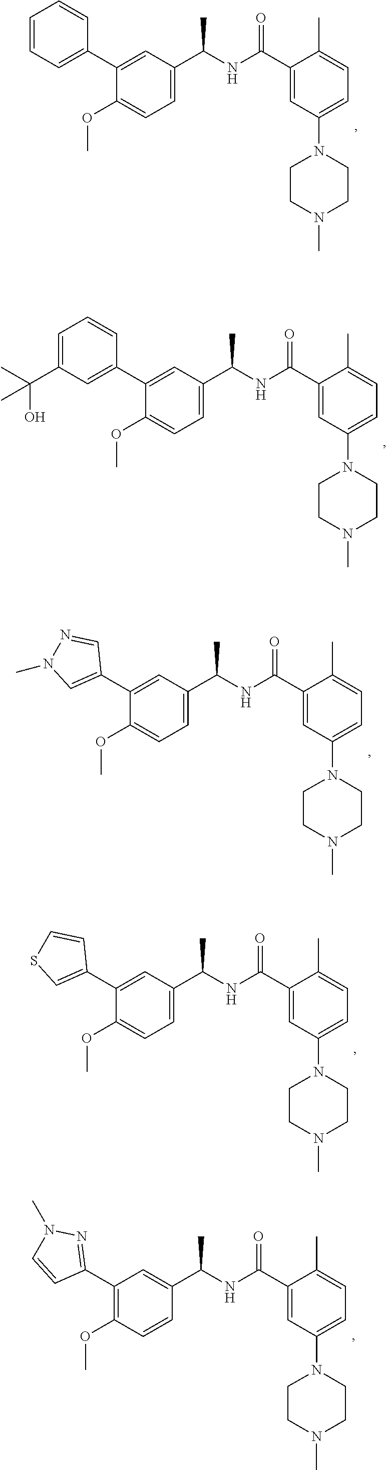 [SMILES: COc1ccc([C@@H](C)NC(=O)c2cc(N3CCN(C)CC3)ccc2C)cc1-c1ccccc1 ; COc1ccc([C@@H](C)NC(=O)c2cc(N3CCN(C)CC3)ccc2C)cc1-c1cccc(C(C)(C)O)c1 ; COc1ccc([C@@H](C)NC(=O)c2cc(N3CCN(C)CC3)ccc2C)cc1-c1cnn(C)c1 ; COc1ccc([C@@H](C)NC(=O)c2cc(N3CCN(C)CC3)ccc2C)cc1-c1ccsc1 ; COc1ccc([C@@H](C)NC(=O)c2cc(N3CCN(C)CC3)ccc2C)cc1-c1ccn(C)n1]

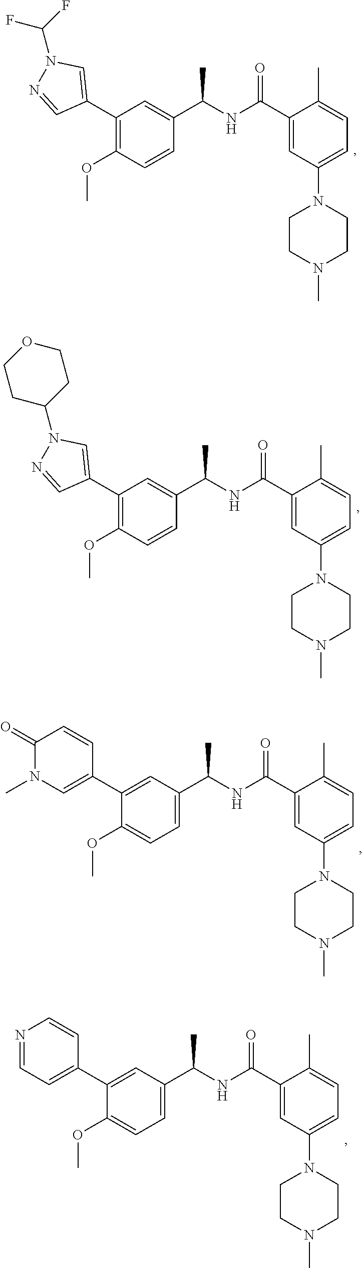 [SMILES: COc1ccc([C@@H](C)NC(=O)c2cc(N3CCN(C)CC3)ccc2C)cc1-c1cnn(C(F)F)c1 ; COc1ccc([C@@H](C)NC(=O)c2cc(N3CCN(C)CC3)ccc2C)cc1-c1cnn(C2CCOCC2)c1 ; COc1ccc([C@@H](C)NC(=O)c2cc(N3CCN(C)CC3)ccc2C)cc1-c1ccc(=O)n(C)c1 ; COc1ccc([C@@H](C)NC(=O)c2cc(N3CCN(C)CC3)ccc2C)cc1-c1ccncc1]

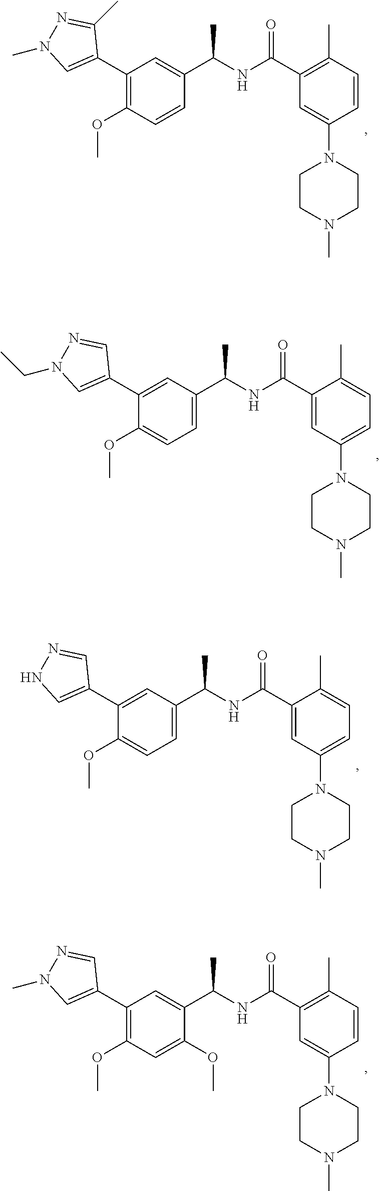 [SMILES: COc1ccc([C@@H](C)NC(=O)c2cc(N3CCN(C)CC3)ccc2C)cc1-c1cn(C)nc1C ; CCn1cc(-c2cc([C@@H](C)NC(=O)c3cc(N4CCN(C)CC4)ccc3C)ccc2OC)cn1 ; COc1ccc([C@@H](C)NC(=O)c2cc(N3CCN(C)CC3)ccc2C)cc1-c1cn[nH]c1 ; COc1cc(OC)c([C@@H](C)NC(=O)c2cc(N3CCN(C)CC3)ccc2C)cc1-c1cnn(C)c1]

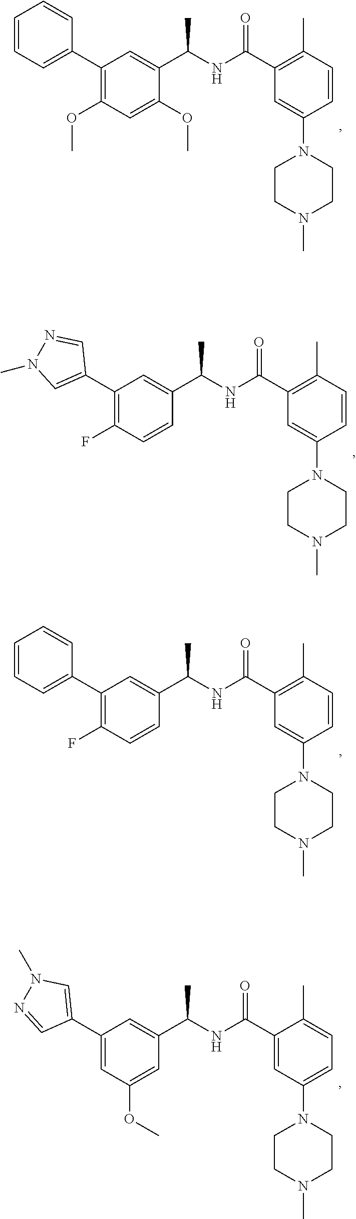 [SMILES: COc1cc(OC)c([C@@H](C)NC(=O)c2cc(N3CCN(C)CC3)ccc2C)cc1-c1ccccc1 ; Cc1ccc(N2CCN(C)CC2)cc1C(=O)N[C@H](C)c1ccc(F)c(-c2cnn(C)c2)c1 ; Cc1ccc(N2CCN(C)CC2)cc1C(=O)N[C@H](C)c1ccc(F)c(-c2ccccc2)c1 ; COc1cc(-c2cnn(C)c2)cc([C@@H](C)NC(=O)c2cc(N3CCN(C)CC3)ccc2C)c1]

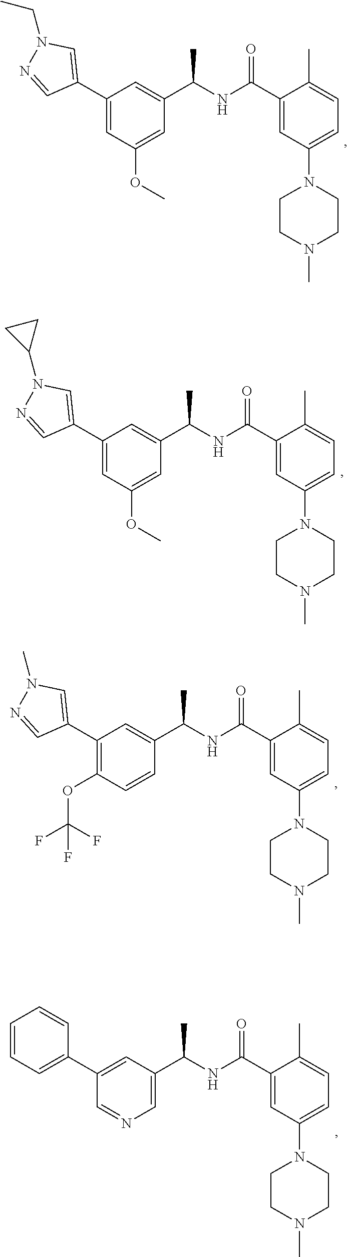 [SMILES: CCn1cc(-c2cc(OC)cc([C@@H](C)NC(=O)c3cc(N4CCN(C)CC4)ccc3C)c2)cn1 ; COc1cc(-c2cnn(C3CC3)c2)cc([C@@H](C)NC(=O)c2cc(N3CCN(C)CC3)ccc2C)c1 ; Cc1ccc(N2CCN(C)CC2)cc1C(=O)N[C@H](C)c1ccc(OC(F)(F)F)c(-c2cnn(C)c2)c1 ; Cc1ccc(N2CCN(C)CC2)cc1C(=O)N[C@H](C)c1cncc(-c2ccccc2)c1]

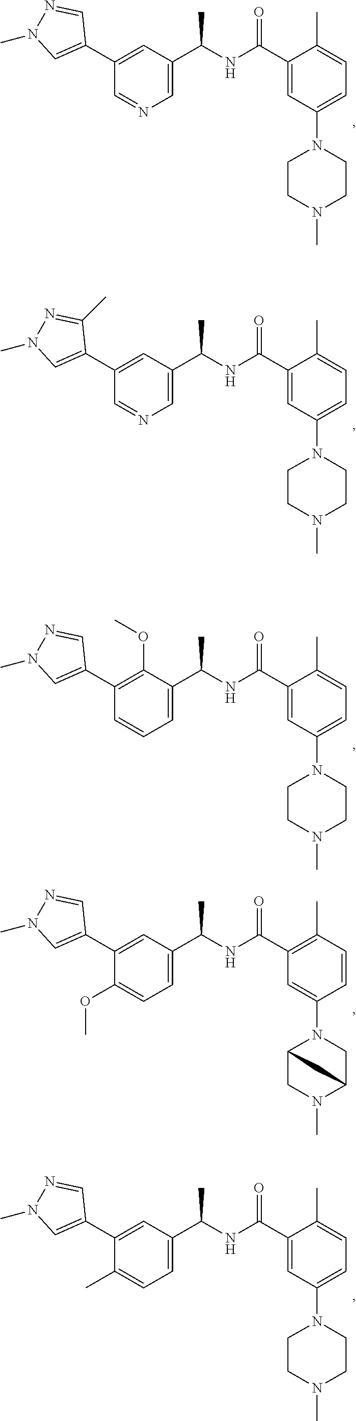

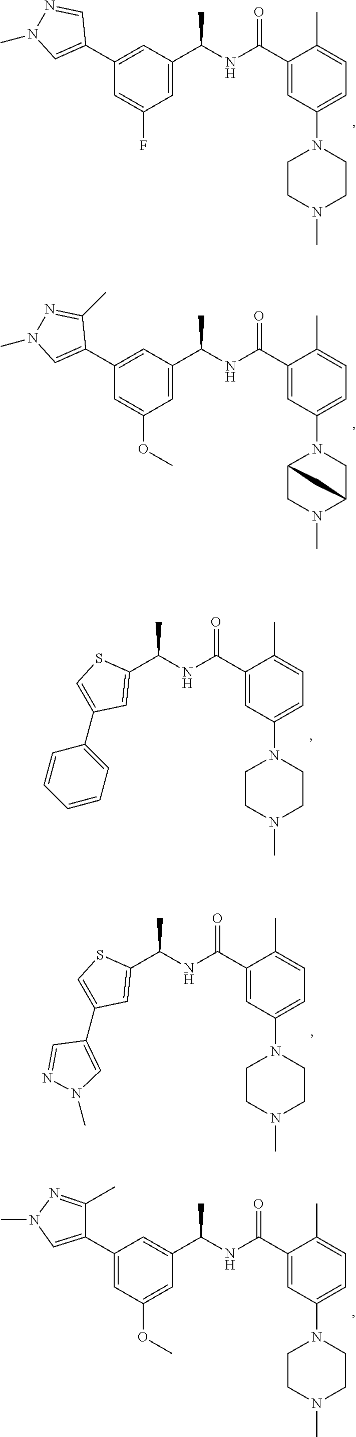 [SMILES: Cc1ccc(N2CCN(C)CC2)cc1C(=O)N[C@H](C)c1cc(F)cc(-c2cnn(C)c2)c1 ; COc1cc(-c2cn(C)nc2C)cc([C@@H](C)NC(=O)c2cc(N3C[C@H]4C[C@@H]3CN4C)ccc2C)c1 ; Cc1ccc(N2CCN(C)CC2)cc1C(=O)N[C@H](C)c1cc(-c2ccccc2)cs1 ; Cc1ccc(N2CCN(C)CC2)cc1C(=O)N[C@H](C)c1cc(-c2cnn(C)c2)cs1 ; COc1cc(-c2cn(C)nc2C)cc([C@@H](C)NC(=O)c2cc(N3CCN(C)CC3)ccc2C)c1]

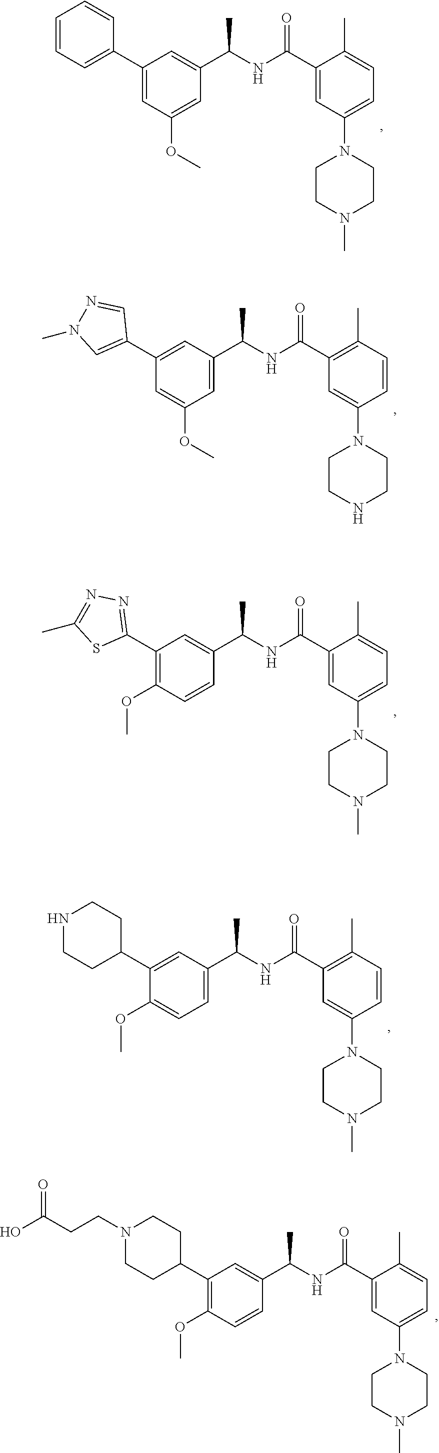 [SMILES: COc1cc(-c2ccccc2)cc([C@@H](C)NC(=O)c2cc(N3CCN(C)CC3)ccc2C)c1 ; COc1cc(-c2cnn(C)c2)cc([C@@H](C)NC(=O)c2cc(N3CCNCC3)ccc2C)c1 ; COc1ccc([C@@H](C)NC(=O)c2cc(N3CCN(C)CC3)ccc2C)cc1-c1nnc(C)s1 ; COc1ccc([C@@H](C)NC(=O)c2cc(N3CCN(C)CC3)ccc2C)cc1C1CCNCC1 ; COc1ccc([C@@H](C)NC(=O)c2cc(N3CCN(C)CC3)ccc2C)cc1C1CCN(CCC(=O)O)CC1]

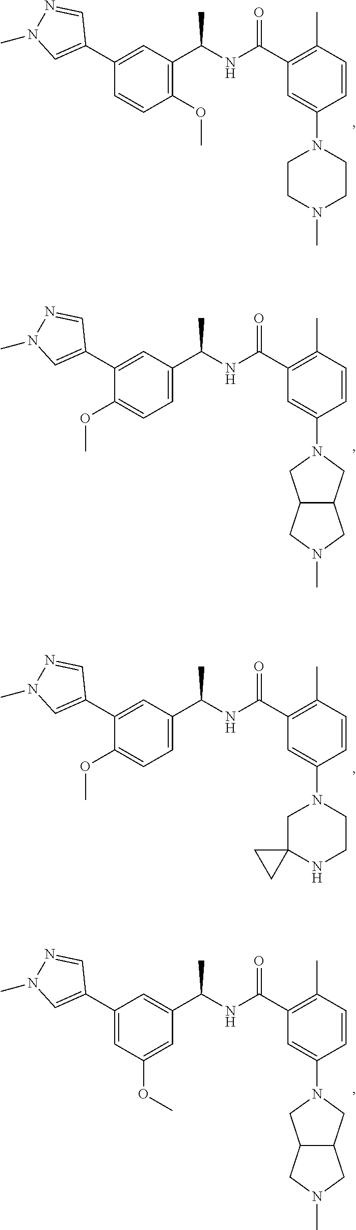 [SMILES: COc1ccc(-c2cnn(C)c2)cc1[C@@H](C)NC(=O)c1cc(N2CCN(C)CC2)ccc1C ; COc1ccc([C@@H](C)NC(=O)c2cc(N3CC4CN(C)CC4C3)ccc2C)cc1-c1cnn(C)c1 ; COc1ccc([C@@H](C)NC(=O)c2cc(N3CCNC4(CC4)C3)ccc2C)cc1-c1cnn(C)c1 ; COc1cc(-c2cnn(C)c2)cc([C@@H](C)NC(=O)c2cc(N3CC4CN(C)CC4C3)ccc2C)c1]

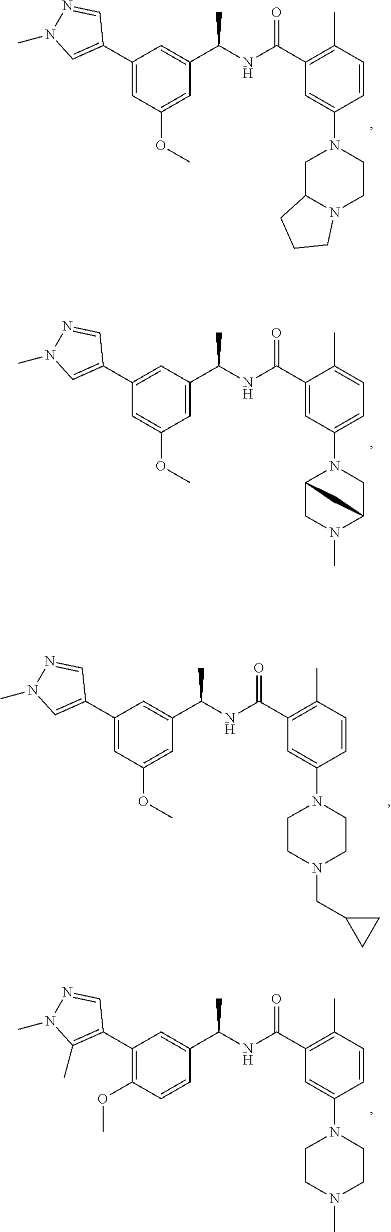 [SMILES: COc1cc(-c2cnn(C)c2)cc([C@@H](C)NC(=O)c2cc(N3CCN4CCCC4C3)ccc2C)c1 ; COc1cc(-c2cnn(C)c2)cc([C@@H](C)NC(=O)c2cc(N3C[C@H]4C[C@@H]3CN4C)ccc2C)c1 ; COc1cc(-c2cnn(C)c2)cc([C@@H](C)NC(=O)c2cc(N3CCN(CC4CC4)CC3)ccc2C)c1 ; COc1ccc([C@@H](C)NC(=O)c2cc(N3CCN(C)CC3)ccc2C)cc1-c1cnn(C)c1C]

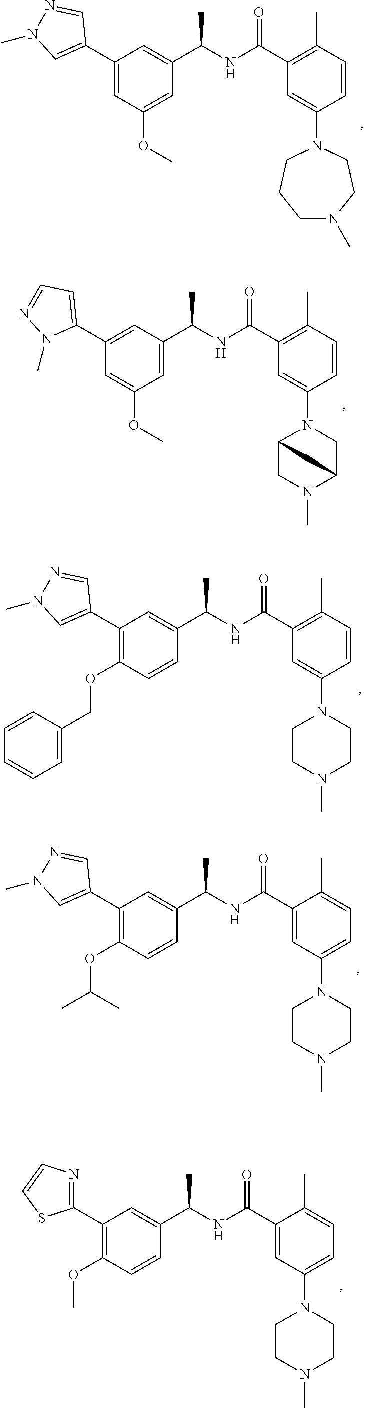 [SMILES: COc1cc(-c2cnn(C)c2)cc([C@@H](C)NC(=O)c2cc(N3CCCN(C)CC3)ccc2C)c1 ; COc1cc(-c2ccnn2C)cc([C@@H](C)NC(=O)c2cc(N3C[C@H]4C[C@@H]3CN4C)ccc2C)c1 ; Cc1ccc(N2CCN(C)CC2)cc1C(=O)N[C@H](C)c1ccc(OCc2ccccc2)c(-c2cnn(C)c2)c1 ; Cc1ccc(N2CCN(C)CC2)cc1C(=O)N[C@H](C)c1ccc(OC(C)C)c(-c2cnn(C)c2)c1 ; COc1ccc([C@@H](C)NC(=O)c2cc(N3CCN(C)CC3)ccc2C)cc1-c1nccs1]

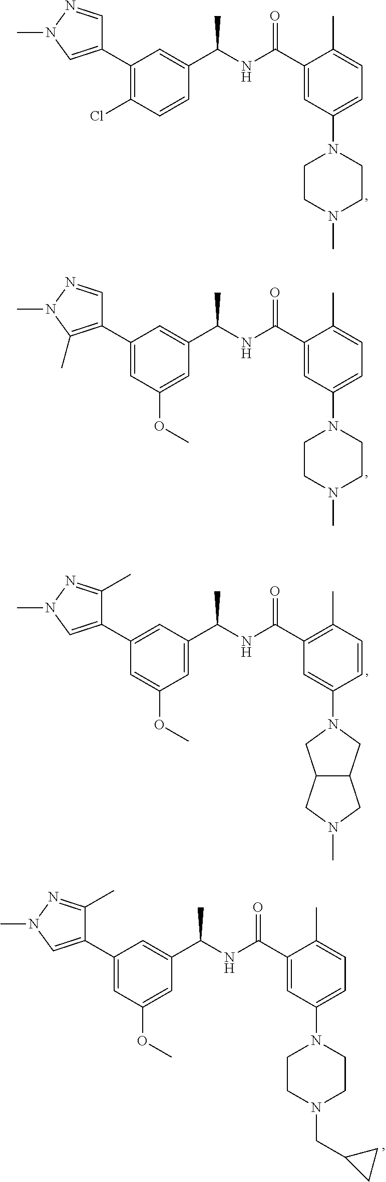

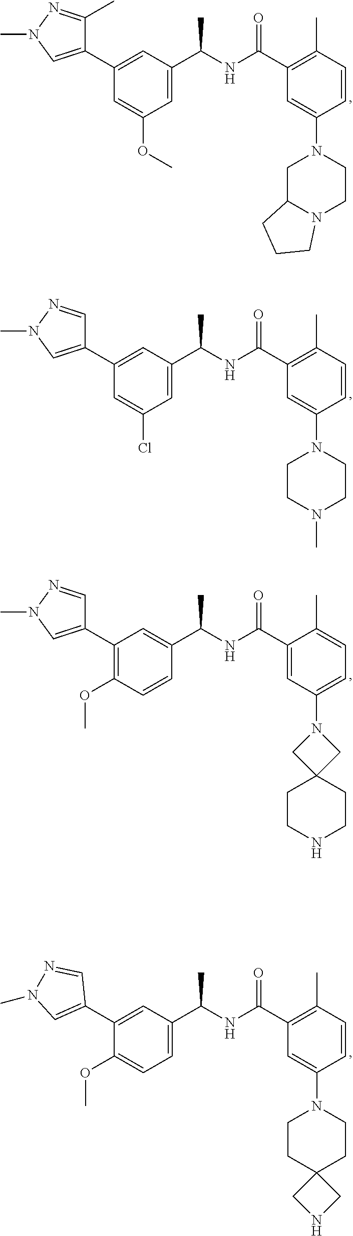

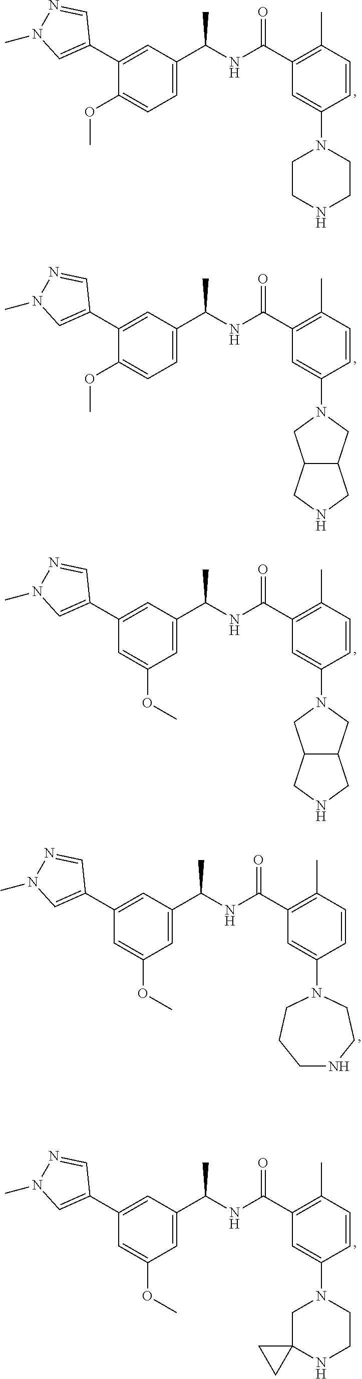

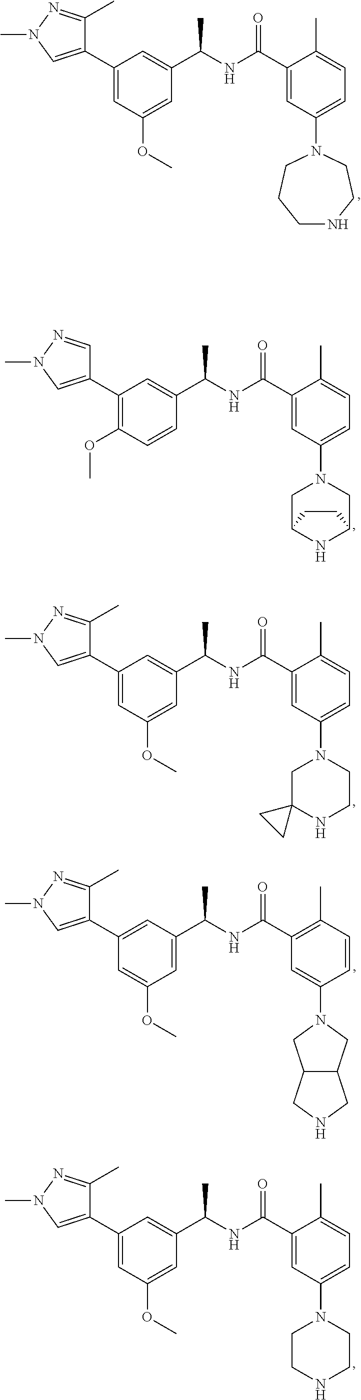

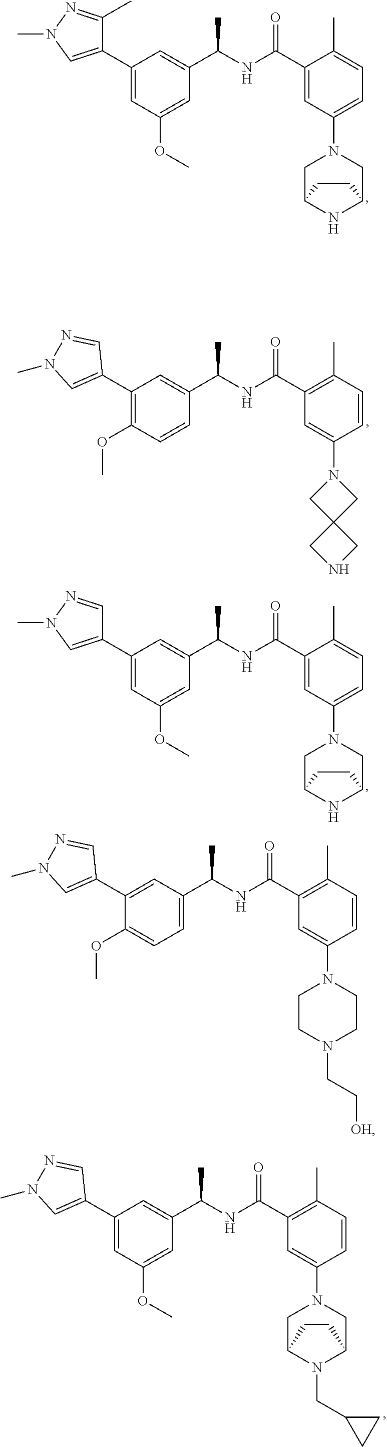

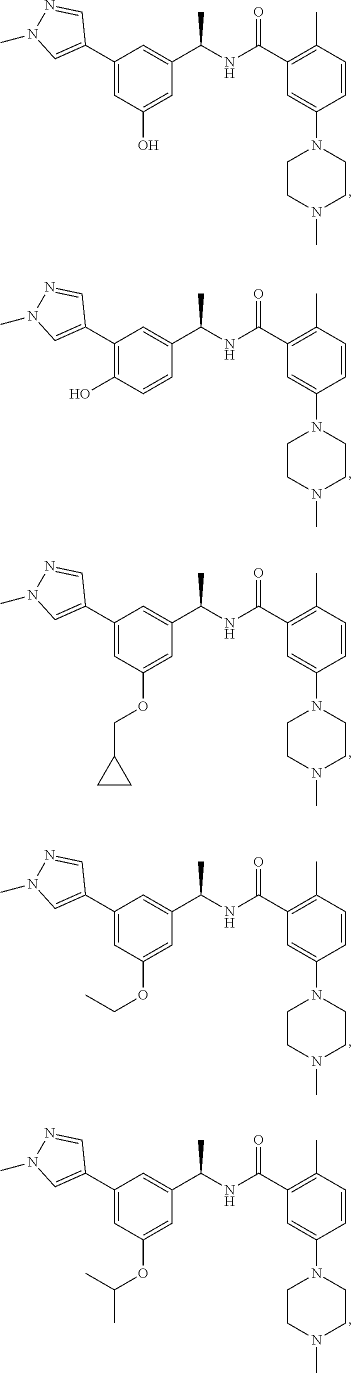

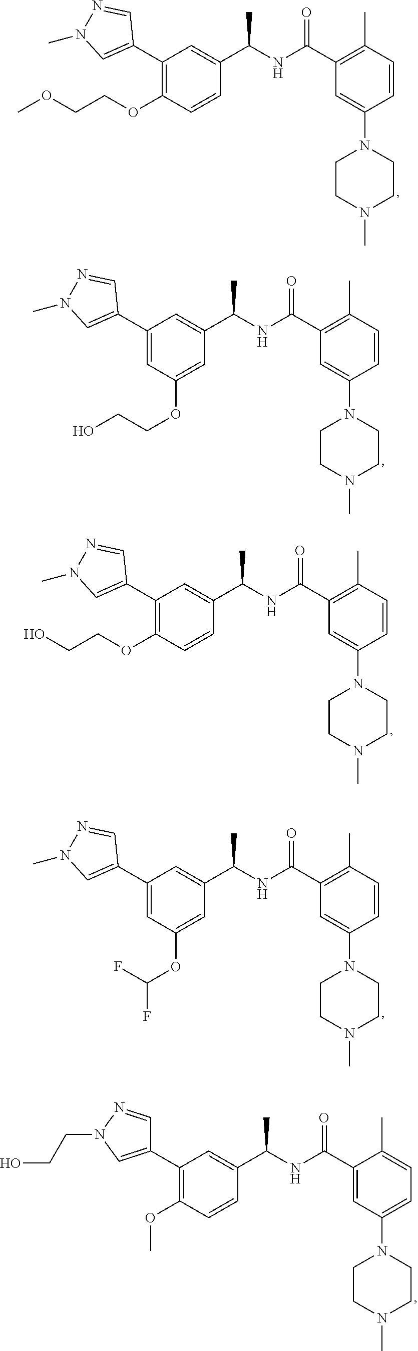

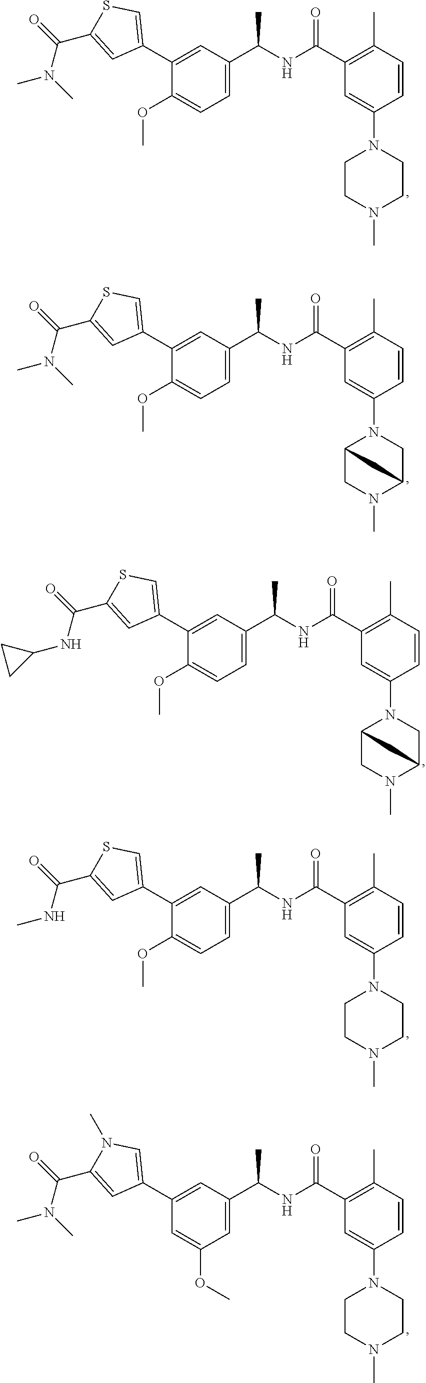

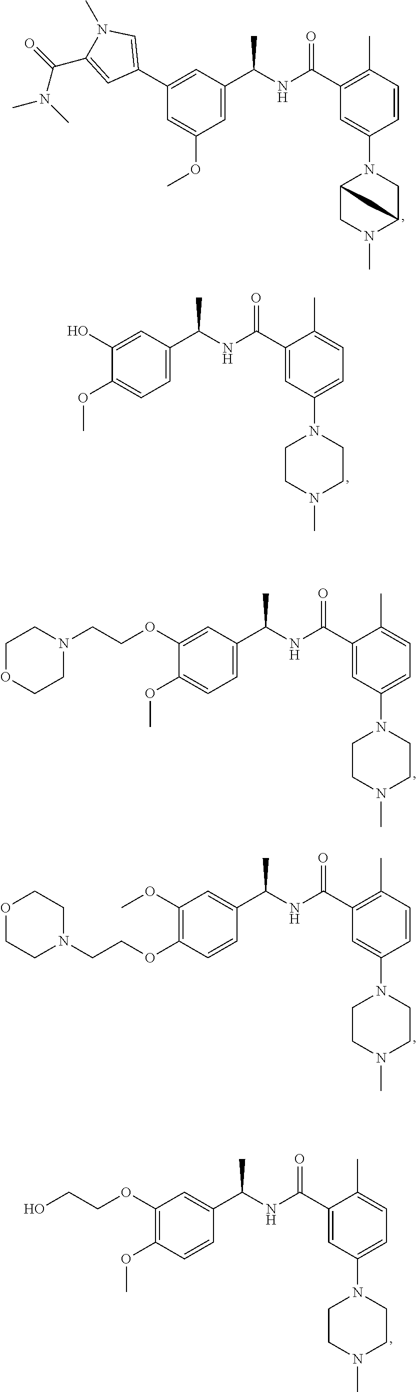

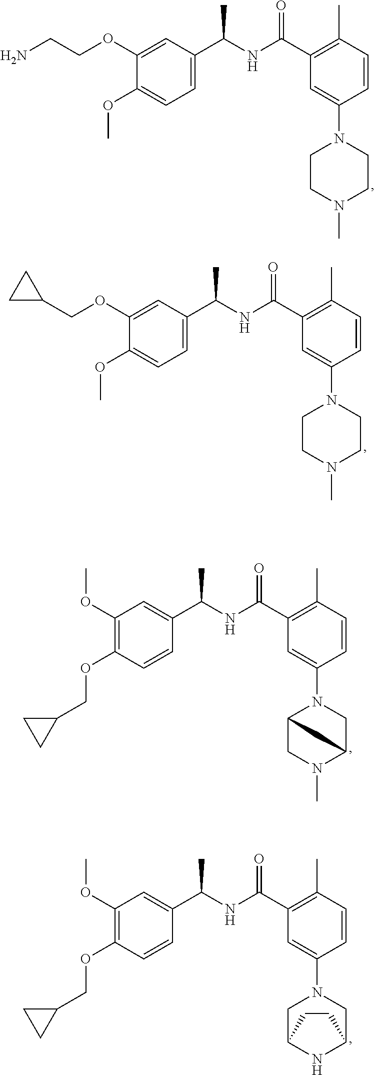

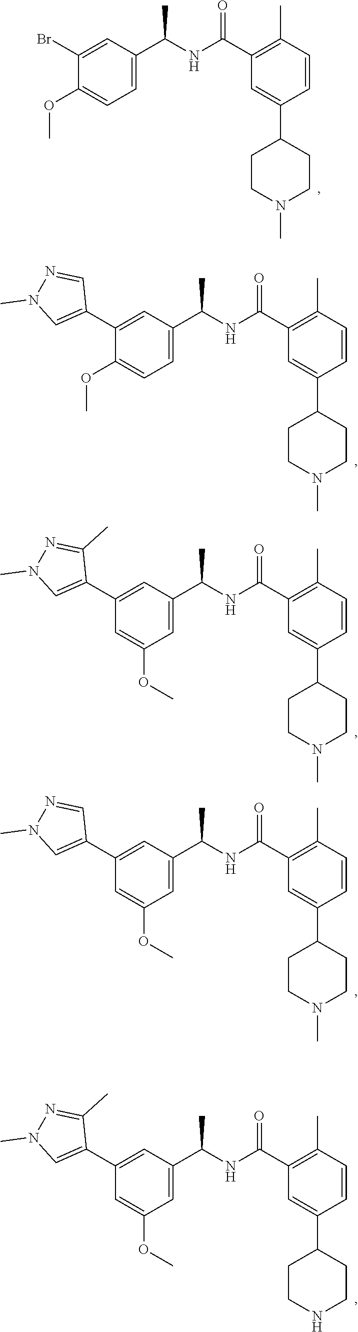

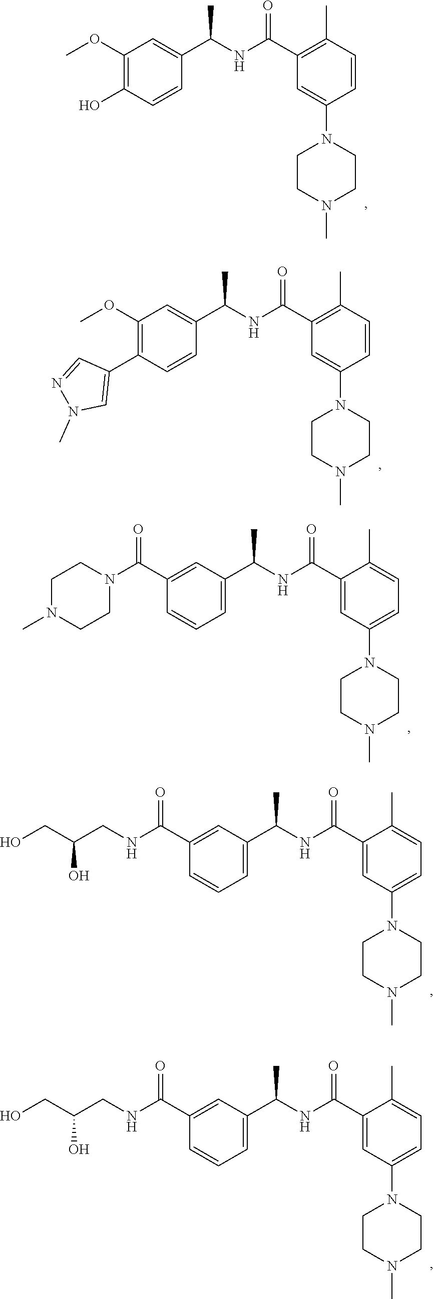

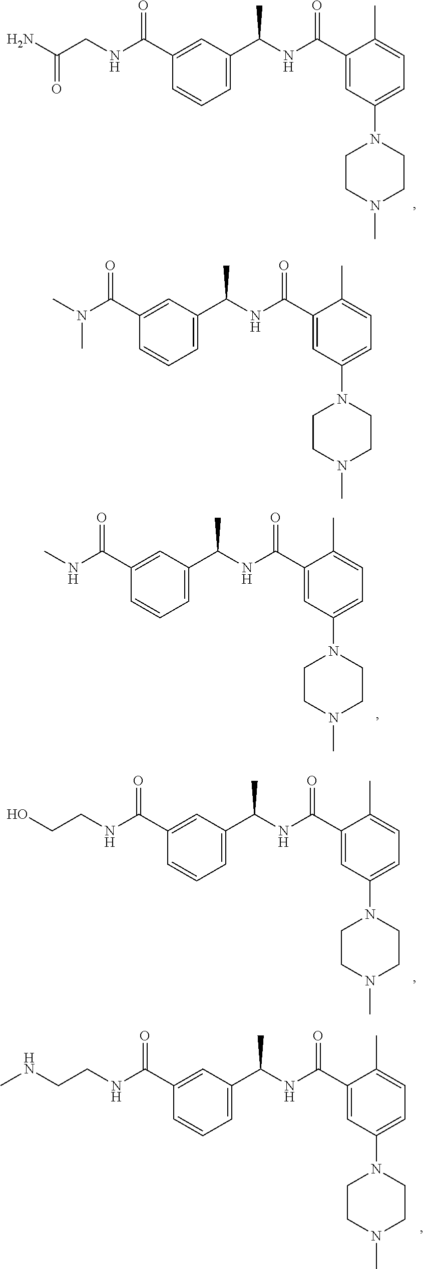

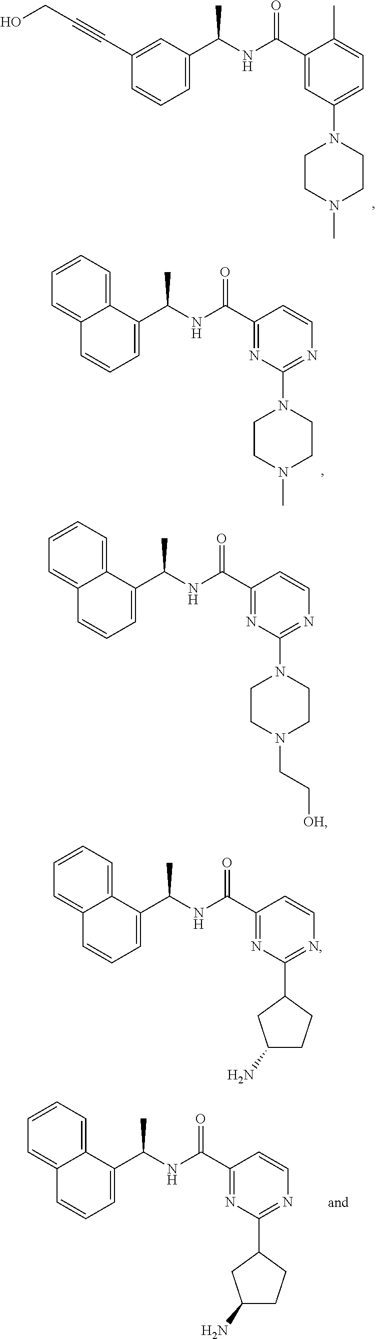 [SMILES: Cc1ccc(N2CCN(C)CC2)cc1C(=O)N[C@H](C)c1cccc(C#CCO)c1 ; C[C@@H](NC(=O)c1ccnc(N2CCN(C)CC2)n1)c1cccc2ccccc12 ; C[C@@H](NC(=O)c1ccnc(N2CCN(CCO)CC2)n1)c1cccc2ccccc12 ; C[C@@H](NC(=O)c1ccnc(C2CC[C@H](N)C2)n1)c1cccc2ccccc12 ; C[C@@H](NC(=O)c1ccnc(C2CC[C@@H](N)C2)n1)c1cccc2ccccc12 ; "and"]

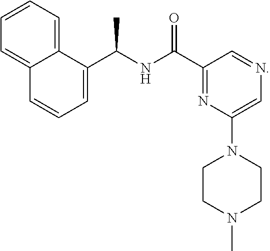 [SMILES: C[C@@H](NC(=O)c1cncc(N2CCN(C)CC2)n1)c1cccc2ccccc12]

16. A pharmaceutical composition comprising a compound of any of claims 1 to 15, or a pharmaceutically acceptable salt thereof, in association with one or more pharmaceutically acceptable excipients.

17. The compound of any of claims 1 to 15, or the composition of claim 16, for use in the inhibition of PLpro activity.

18. A compound of any of claims 1 to 15, a pharmaceutically acceptable salt thereof, or a composition of claim 16, for use in the treatment of a viral infection.

19. The compound for use of claim 18, wherein the viral infection is a disease or disorder caused by coronaviruses, rotaviruses, noroviruses, enteroviruses, hepatitis viruses (e.g. HAV, HBV, HCV, HDV, HEV), herpesviruses, papillomaviruses, arboviruses (e.g. West Nile virus, Zika virus, Dengue virus), ebolaviruses, rabies virus, or rubella virus.

20. The compound or composition for use of claim 19, wherein the disease or disorder is caused by coronaviruses.

21. The compound or composition for use of claim 20, wherein the disease or disorder is selected from: coronavirus disease 2019 (COVID-19), severe acute respiratory syndrome (SARS), Middle East respiratory syndrome (MERS), common cold, or other coronavirus infections.
